(19) United States
(12) Patent Application Publication
(10) Pub. No.: US 2018/0072997 A1
(43) Pub. Date: Mar. 15, 2018

(54) INHIBITORS OF HUMAN IMMUNODEFICIENCY VIRUS REPLICATION

(71) Applicant: ViiV HEATHCARE UK (NO.5) LIMITED, Brentford, Middlesex (GB)

(72) Inventors: John A. BENDER, Wallingford, CT (US); Omar D. LOPEZ, Wallingford, CT (US); Van N. NGUYEN, Auburn, MI (US); Zhong YANG, Southington, CT (US); Alan Xiangdong WANG, Wallingford, CT (US); Gan WANG, Cheshire, CT (US); Nicholas A. MEANWELL, Wallingford, CT (US); Brett R. BENO, Wallingford, CT (US); Robert A. FRIDELL, Wallingford, CT (US); Makonen BELEMA, Wallingford, CT (US); Srinivasan THANGATHIRUPATHY, Bangalore, Karnataka (IN)

(21) Appl. No.: 15/565,716

(22) PCT Filed: Apr. 22, 2016
(86) PCT No.: PCT/US2016/028763

§ 371 Date: Oct. 11, 2017

Related U.S. Application Data
(60) Provisional application No. 62/151,790, filed on Apr. 23, 2015.

(51) Int. Cl. C12N 7/06; C07C 237/22; C07C 271/22; C07C 275/24; C07C 307/06; C07C 311/06; C07C 311/19; C07D 213/61; C07D 235/26; C07D 271/12; C07D 277/62; C07D 285/14; C07D 295/192; C07D 317/66; C07K 5/02

(52) U.S. Cl. CPC C12N 7/06; C12N 2740/16063; C07C 271/22; C07C 275/24; C07C 307/06; C07C 311/06; C07C 311/19; C07D 213/61; C07D 235/26; C07D 271/12; C07D 277/62; C07D 285/14; C07D 295/192; C07D 317/66; C07K 5/022; C07C 2601/08; C07C 2601/10; C07C 2601/14; C07C 237/22

(57) ABSTRACT

Compounds of Formulas I-VI, including pharmaceutically acceptable salts thereof, and compositions and methods for treating human immunodeficiency virus (HIV) infection are set forth. Formula I is exemplified below:

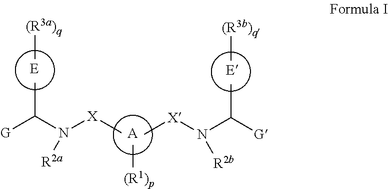

### CROSS REFERENCE TO RELATED APPLICATION

This application claims the benefit of U.S. Provisional Application Ser. No. 62/151,790 filed Apr. 23, 2015 which is herein incorporated by reference in its entirety.

### FIELD OF THE INVENTION

The invention relates to compounds, compositions, and methods for the treatment of human immunodeficiency virus (HIV) infection. More particularly, the invention provides novel inhibitors of HIV, pharmaceutical compositions containing such compounds, and methods for using these compounds in the treatment of HIV infection. The invention also relates to methods for making the compounds hereinafter described.

### BACKGROUND OF THE INVENTION

Acquired immunodeficiency syndrome (AIDS) is the result of infection by HIV. It remains a major medical problem, with an estimated 34 million people infected worldwide at the end of 2011, 3.3 million of them under the age of 15. In 2011, there were 2.5 million new infections, and 1.7 million deaths from complications due to HIV/AIDS.

Current therapy for HIV-infected individuals consists of a combination of approved anti-retroviral agents. Over two dozen drugs are currently approved for HIV infection, either as single agents or as fixed dose combinations or single tablet regimens, the latter two containing 2-4 approved agents. These agents belong to a number of different classes, targeting either a viral enzyme or the function of a viral protein during the virus replication cycle. Thus, agents are classified as either nucleotide reverse transcriptase inhibitors (NRTIs), non-nucleotide reverse transcriptase inhibitors (NNRTIs), protease inhibitors (PIs), integrase inhibitors (INIs), or entry inhibitors (one, maraviroc, targets the host CCR5 protein, while the other, enfuvirtide, is a peptide that targets the gp41 region of the viral gp160 protein). In addition, a pharmacokinetic enhancer with no antiviral activity, i.e., cobicistat, available from Gilead Sciences, Inc. under the tradename TYBOST™ (cobicistat) tablets, has recently been approved for use in combinations with certain antiretroviral agents (ARVs) that may benefit from boosting.

Despite the armamentarium of agents and drug combinations, there remains a medical need for new anti-retroviral agents, due in part to the need for chronic dosing to combat infection. Significant problems related to long-term toxicities are documented, creating a need to address and prevent these co-morbidities (e.g. CNS, CV/metabolic, renal disease). Also, increasing failure rates on current therapies continue to be a problem, due either to the presence or emergence of resistant strains or to non-compliance attributed to drug holidays or adverse side effects. For example, despite therapy, it has been estimated that 63% of subjects receiving combination therapy remained viremic, as they had viral loads >500 copies/mL (Oette, M, Kaiser, R, Daumer, M, et al. Primary HIV Drug Resistance and Efficacy of First-Line Antiretroviral Therapy Guided by Resistance Testing. J Acq Imm Def Synd 2006; 41(5):573-581). Among these patients, 76% had viruses that were resistant to one or more classes of antiretroviral agents. As a result, new drugs are needed that are easier to take, have high genetic barriers to the development of resistance and have improved safety over current agents. In this panoply of choices, novel MOAs that can be used as part of the preferred highly active antiretroviral therapy (HAART) regimen can still have a major role to play since they should be effective against viruses resistant to current agents.

Certain therapeutic compounds are disclosed in WO 2013/006738, WO 2014/110298, and WO 2014/134566.

What is now needed in the art are additional compounds which are novel and useful in the treatment of HIV. Additionally, these compounds may desireably provide advantages for pharmaceutical uses, for example, with regard to one or more of their mechanisms of action, binding, inhibition efficacy, target selectivity, solubility, safety profiles, or bioavailability. Also needed are new formulations and methods of treatment which utilize these compounds.

### SUMMARY OF THE INVENTION

The invention encompasses compounds of the invention, e.g., set forth in Formula I below and Formulas II-VI (hereinafter described), including pharmaceutically acceptable salts, their pharmaceutical compositions, and their use in inhibiting HIV and treating those infected with HIV or AIDS.

One aspect of the invention encompasses a compound of Formula I, including pharmaceutically acceptable salts thereof:

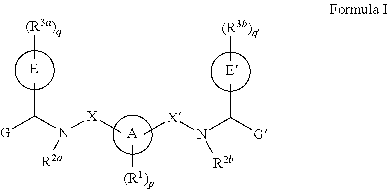

wherein:
A is a bond or is selected from C₁-C₅ alkyl, C₂-C₅ alkenyl, C₂-C₅ alkynyl, aryl, C₃-C₆ cycloalkyl, C₂-C₅ bicycloalkyl, —CO—, —CS—, —C(═N—CN)—, heterocyclyl, nitrogen, sulfur, oxygen, —O—(C₂-C₄ alkyl)-O—, —N(R^{xa})CON(R^{xb})—, and ferrocene;
   each R¹ is independently selected from hydrogen, C₁-C₄ alkyl, C₂-C₄ alkenyl, C₁-C₄ alkoxy, C₂-C₄ (alkoxyalkyl), (C₁-C₄ alkoxy)carbonyl, C₁-C₄ alkylthioxy, benzyloxy, C₂-C₄ alkynyl, aryl, carboxylic acid, cyano, halogen, C₁-C₄ haloalkyl, C₁-C₄ haloalkoxy, heterocyclyl, hydroxy, C₁-C₄ hydroxyalkyl, thioxy, —CH₂NH₂, —(C₁-C₄ alkyl)-heteroaryl, —CO—(C₁-C₄ alkyl), —CO(R^{y}), —CON(R^{xa})₂, —NHCON(R^{xa})₂, —NHCO—(C₁-C₄ alkyl), —NHCO₂—(C₁-C₄ alkyl), —NHSO₂—(C₁-C₄ alkyl), —OCH₂-aryl, —SO₂—(C₁-C₄ alkyl), —SO₂—N(R^{xa})₂, —SO₂-heterocyclyl, —N(R^{xa})₂, and nitro;
p is from 0 to 5;
R^{xa} and R^{xb} are independently selected from hydrogen, alkyl, or haloalkyl;
R^{y} is selected from C₂-C₄ (dialkylamine) or nitrogen-containing heterocyclyl, and is attached to the parent fragment through its nitrogen;
X and X¹ are each are independently a bond or are selected from:

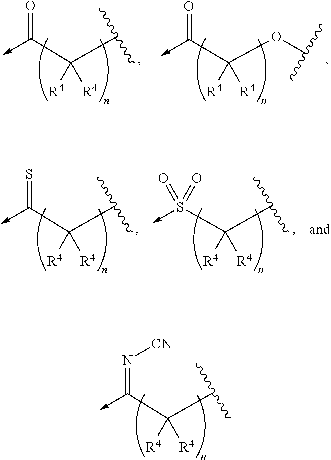

wherein the attachment of X and X¹ to the parent structure is such that the bond with the arrow is oriented toward the respective nitrogen shown in Formula I; provided, however, that when A is a bond, at least one X or X¹ is not a bond;
each n is independently from 0 to 2;
each R⁴ is independently selected from hydrogen, C₁-C₃ alkyl, C₁-C₃ alkenyl, aryl, aryl(C₁-C₂ alkyl), hydroxyl, and halogen, with the option for two R⁴ on the same or adjacent carbon(s) to form a ring;
R^{2a} and R^{2b} are independently selected from hydrogen, C₁-C₄ alkyl, C₃-C₄ alkenyl, C₃-C₅ alkynyl and C₃-C₄ cycloalkyl, and each is optionally substituted with 1 to 3 substituents selected from halogen, hydroxyl, C₁-C₂ alkoxy, and C₁-C₂ haloaloxy;
G and G′ are each independently selected from;

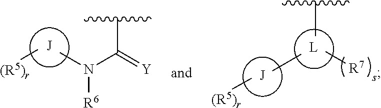

each Y is independently oxygen or sulfur;
each J is a bond or is independently selected from aryl, heterocyclyl, or C₃-C₇ cycloalkyl;
each R⁵ is independently selected from hydrogen, C₁-C₄ alkoxy, C₁-C₄ alkyl, halogen, C₂-C₅ bicycloalkyl, C₁-C₄ haloalkoxy, C₁-C₄ haloalkyl, —CONH₂, —CN, —NHCO(C₁-C₄ alkyl), —NHCON(C₁-C₄ alkyl)₂, —NHCO₂(C₁-C₄ alkyl), —OH, —SO₂N(C₁-C₄alkyl)₂ and heterocyclyl;
each r is independently from 0 to 5;
each R⁶ is independently selected from hydrogen, C₁-C₄ alkyl, C₂-C₄ alkenyl, and C₃-C₄ cycloalkyl, optionally substituted with halogen, hydroxyl, C₁-C₂ alkoxy, or C₁-C₂ haloalkoxy;
each L is independently selected from a five or six-member heteroaryl ring;
each R⁷ is independently selected from C₁-C₃ alkoxy, C₁-C₃ alkyl, halogen, C₁-C₃ haloalkoxy, C₁-C₃ haloalkyl, —CONH₂, —CN, —OH, —C₂-C₅ alkynol, —NHCO(C₁-C₃ alkyl), —NHCON(C₁-C₃ alkyl)₂, —NHCO₂(C₁-C₃ alkyl), —SO₂N(C₁-C₃alkyl)₂, and C₂-C₆ alkyne optionally substituted with 1 to 2 halides;
each s is independently from 0 to 4;
E and E′ are each independently selected from C₁-C₈ alkyl, C₂-C₈ alkenyl, C₂-C₈ alkynyl, C₅-C₈ bicycloalkyl, C₃-C₇ cycloalkyl, aryl, heterocyclyl, and a C₁-C₂ alkyl group containing any one of the following groups: C₅-C₈ bicycloalkyl, C₃-C₇ cycloalkyl, aryl, and heterocyclyl;
R^{3a} and R^{3b} are each independently selected from C₂-C₄ alkenoxy, C₂-C₄ alkenyl, C₁-C₄ alkoxy, C₂-C₄ (alkoxyalkyl), (C₁-C₄alkoxy)carbonyl, C₁-C₄ alkyl, C₁-C₄ haloalkyl, C₁-C₄ haloalkoxy, carboxyamide, halogen, —CN, —NHCO(C₁-C₄ alkyl), —OH, C₁-C₄ hydroxyalkyl, and —SO₂N-heterocycle; and
q and q′ are each independently from 0 to 5;
wherein the attachment of each of “X”, “X¹” or N to “A” could be on the same or different atom(s) of “A”.

In an aspect of the invention, A is a bond and at least one of X and X¹ are independently selected from:

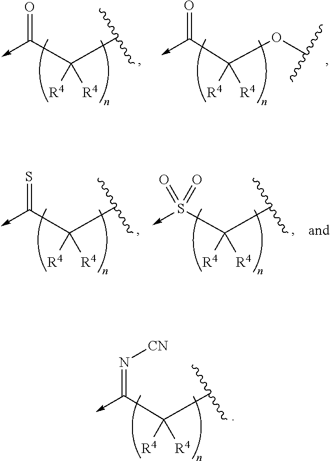

In an aspect of the invention, A is selected from C₁-C₅ alkyl, C₂-C₅ alkenyl, C₂-C₅ alkynyl, aryl, C₃-C₆ cycloalkyl, C₂-C₅ bicycloalkyl, —CO—, —CS—, —C(═N—CN)—, heterocyclyl, nitrogen, sulfur, oxygen, —O—(C₂-C₄ alkyl)-O—, —N(R^{xa})CON(R^{xb})—, and ferrocene. In an aspect of the invention, at least one of X and X¹ are a bond.

The invention also relates to pharmaceutical compositions comprising the compounds of the invention, including pharmaceutically acceptable salts thereof, and a pharmaceutically acceptable carrier, excipient, and/or diluent.

In addition, the invention provides one or more methods of treating HIV infection comprising administering a therapeutically effective amount of the compounds of the invention to a patient.

Also provided as part of the invention are one or more methods for making the compounds of the invention.

The present invention is directed to these, as well as other important ends, hereinafter described.

### DETAILED DESCRIPTION OF THE INVENTION

The singular forms “a”, “an”, and “the” include plural reference unless the context dictates otherwise.

In all circumstances, where a given group is noted to exist more than once, as in the alkyl group in NHCON(alkyl)₂ or dialkylamine, it is understood that the repeat versions of the group in the molecule can be selected independently of each other.

Where appropriate, when a substituent is not specified, it is understood that it is hydrogen.

Unless otherwise expressly set forth elsewhere in the application, the following terms shall have the following meanings:

“Alkenyl” means a straight or branched alkyl group comprised of 2 to 10 carbons with at least one double bond and optionally substituted with 0-3 halo or alkoxy group.

“Alkenyloxy” means an alkenyl group attached to the parent structure by an oxygen atom.

“Alkoxy” means an alkyl group attached to the parent structure by an oxygen atom.

“Alkoxycarbonyl” means an alkoxy group attached to the parent structure by a carbonyl moiety.

“Alkyl” means a straight or branched saturated hydrocarbon comprised of 1 to 10 carbons, and preferably 1 to 6 carbons.

“Alkylthioxy” or “alkyl-S—” means an alkyl group attached to the parent structure through a sulfur atom.

“Alkynol” means a hydrocarbon containing both a triple bond and an alcohol group.

“Alkynyl” means a straight or branched alkyl group comprised of 2 to 10 carbons, preferably 3 to 6 carbons, containing at least one triple bond and optionally substituted with 0-3 halo or alkoxy group.

“Aryl” means a carbocyclic group comprised of 1-3 rings that are fused and/or bonded and at least one or a combination of which is aromatic. The non-aromatic carbocyclic portion, where present, will be comprised of C₃ to C₇ alkyl group. Examples of aromatic group include, but are not limited to, phenyl, biphenyl, cyclopropylphenyl, indane, naphthalene, and tetrahydronaphthalene. The aryl group can be attached to the parent structure through any substitutable carbon atom in the group.

“Arylalkyl” is a C₁-C₅ alkyl group attached to 1 to 2 aryl groups and linked to the parent structure through the alkyl moiety. Examples include, but are not limited to, —(CH₂)ₙPh with n=1-5, —CH(CH₃)Ph, —CH(Ph)₂.

“Aryloxy” is an aryl group attached to the parent structure by oxygen.

“Azaindoline” means one of the aromatic “CH” moieties of an indoline is substituted with a nitrogen atom.

“Azatetrahydroquinoline” means any aromatic CH moiety of tetrahydroquinoline is substituted with a nitrogen atom.

“Benzyloxy” means a benzyl group is attached to the parent structure through an oxygen atom. The phenyl group of the benzyl moiety could be optionally substituted by 1-3 moieties independently selected from the group of alkyl, alkoxy, halo, haloalkyl, haloalkoxy and cyano.

“Cₓ-C_{y}” notation indicates a structural element comprised of carbons numbering between ‘x’ and ‘y’. For example, “C₅-C₁₀ bicycloalkyl” means a bicyclic ring system comprised of 5 to 10 carbons, where the rings are attached in a fused, spiro or bridged manner; an example of C₅-C₁₀ bicycloalkyl include, but is not limited to, bicyclo[2.2.2]octane. Similarly, “C₃-C₄ cycloalkyl” is a subset of monocyclic ring system comprised of 3 to 4 carbons.

“Cycloalkyl” means a monocyclic ring system comprised of 3 to 7 carbons.

“Cyano” refers to —CN.

“Diazaindole” means any two “CH” moieties in the 6-member ring of an indole are substituted with nitrogen atoms.

“Diazaindoline” means any two aromatic “CH” moieties of an indoline are substituted with a nitrogen atom.

“Diazatetrahydroquinoline” means any two aromatic CH moieties of tetrahydroquinoline are substituted with nitrogen atoms.

“Halo” or “halogen” refers to —F, —Cl, —Br, or —I.

“Haloalkyl” means an alkyl group substituted by any combination of one to six halogen atoms.

“Haloalkoxy” or “Haloalkyloxy” means a haloalkyl group attached to the parent structure through an oxygen atom.

“Hydroxy” refers to —OH.

“Heteroaryl” is a subset of heterocyclic group as defined below and is comprised of 1-3 rings where at least one or a combination of which is aromatic and that the aromatic group contains at least one atom chosen from a group of oxygen, nitrogen or sulfur.

“Heterocyclyl or heterocyclic” means a cyclic group of 1-3 rings comprised of carbon and at least one other atom selected independently from the group of oxygen, nitrogen and sulfur. The rings could be bridged, fused and/or bonded, through a direct or spiro attachment, with the option to have one or a combination thereof be aromatic. Examples include, but are not limited to pyridine, pyrimidine, bipyrimidine, pyridazine, pyrazine, triazine, piperizine, pyrazole, thiophene, imidazole, isoxazole, indole, 1,3-dihydrobenzo[c][1,2,5]thiadiazole 2,2-dioxide, 1H-benzo[d]imidazol-2(3H)-one, imidazolidin-2-one, 2,3-dihydrophthalazine-1,4-dione, quinoxaline-2,3(1H,4H)-dione, 3-hydroxyquinoxalin-2(1H)-one, quinazoline-2,4(1H,3H)-dione, benzothiazole, benzothiazolone, benzothiadiazole, benzodioxole, benzoxazolone, benzisothiazole, 1-methylpyridin-2(1H)-one, 2,3-dihydrobenzo[b][1,4]dioxine, indazole, benzimidazole, quinoxaline, azaindole, azetidine, benzothiophene, benzoxazole, chroman, dihydrobenzofuran, dihydro-benzo[1,4]oxazine, 2,3-dihydrobenzo[d]isothiazole 1,1-dioxide, furanylphenyl, imidazo[1,2-a]pyridine, indoline, isoquinoline, isoquinolinone, isothiazolidine 1,1-dioxide, morpholine, oxadiazole-phenyl, phenylaztidine, piperidine, pyridinylphenyl, pyrrolidine, pyrrole, quinazoline, quinoline, tetrahydroisoquinoline, tetrahydroquinoline, triazole, or triazolone. Unless otherwise specifically set forth, the heterocyclic group can be attached to the parent structure through any suitable atom in the group that results in a stable compound.

It is understood that a subset of the noted heterocyclic examples encompass regioisomers. For instance, “azaindole” refers to any of the following regioisomers: 1H-pyrrolo[2,3-b]pyridine, 1H-pyrrolo[2,3-c]pyridine, 1H-pyrrolo[3,2-c]pyridine, and 1H-pyrrolo[3,2-b]pyridine.

“Heterocyclylalkyl” is a heterocyclyl moiety attached to the parent structure through an alkyl group.

“—SO₂N-heterocyclyl” means a nitrogen containing heterocycle that is attached through its nitrogen to SO2 which in turn is attached to the parent structure.

“Tetrahydroquinoline” means 1,2,3,4-tetrahydroquinoline.

Substituents which are illustrated by chemical drawing to bond at variable positions on a multiple ring system (for example a bicyclic ring system) are intended to bond to the ring where they are drawn to append. Parenthetic and multiparenthetic terms are intended to clarify bonding relationships to those skilled in the art. For example, a term such as ((R)alkyl) means an alkyl substituent further substituted with the substituent R; the term “—CO—(C₁-C₄ alkyl)” means an alkyl group comprised of one to four carbons is attached to the parent structure through carbonyl; and, the term “C₂-C₄ (dialkylamine)” means a dialkylamine moiety that is comprised of a total of 2 to 4 carbons. Also, those skilled in the art will recognize that the compounds of the invention may have a variety of substituents as set forth, for example, in Formulas I-VI, to the extent the substitutions are chemically possible. For example, those skilled in the art will recognize that when “A” in Formula I is a bond, then “p” must be “0” in order for R¹ to be absent.

Those terms not specifically set forth herein shall have the meaning which is commonly understood and accepted in the art.

The invention includes all pharmaceutically acceptable salt forms of the compounds. Pharmaceutically acceptable salts are those in which the counter ions do not contribute significantly to the physiological activity or toxicity of the compounds and as such function as pharmacological equivalents. These salts can be made according to common organic techniques employing commercially available reagents. Some anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate. Some cationic salt forms include ammonium, aluminum, benzathine, bismuth, calcium, choline, diethylamine, diethanolamine, lithium, magnesium, meglumine, 4-phenylcyclohexylamine, piperazine, potassium, sodium, tromethamine, and zinc.

Some of the compounds of the invention exist in stereoisomeric forms. The invention includes all stereoisomeric forms of the compounds including enantiomers and diastereromers. Methods of making and separating stereoisomers are known in the art. The invention includes all tautomeric forms of the compounds. The invention includes atropisomers and rotational isomers.

The invention is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. Such compounds may have a variety of potential uses, for example as standards and reagents in determining biological activity. In the case of stable isotopes, such compounds may have the potential to favorably modify biological, pharmacological, or pharmacokinetic properties.

In an aspect of the invention, there is provided a compound of Formula II, including pharmaceutically acceptable salts thereof:

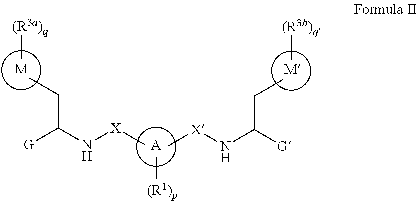

wherein
A is a bond or is selected from C₁-C₅ alkyl, C₂-C₅ alkenyl, C₂-C₅ alkynyl, aryl, C₃-C₆ cycloalkyl, C₂-C₅ bicycloalkyl, —CO—, —CS—, —C(═N—CN)—, heterocyclyl, nitrogen, sulfur, oxygen, —O—(C₂-C₄ alkyl)-O—, —N(R^{xa})CON(R^{xb})—, and ferrocene;
each R¹ is independently selected from hydrogen, C₁-C₄ alkyl, C₂-C₄ alkenyl, C₁-C₄ alkoxy, C₂-C₄ (alkoxyalkyl), (C₁-C₄ alkoxy)carbonyl, C₁-C₄ alkylthioxy, benzyloxy, C₂-C₄ alkynyl, aryl, carboxylic acid, cyano, halogen, C₁-C₄ haloalkyl, C₁-C₄ haloalkoxy, heterocyclyl, hydroxy, C₁-C₄ hydroxyalkyl, thioxy, —CH₂NH₂, —(C₁-C₄ alkyl)-heteroaryl, —CO—(C₁-C₄ alkyl), —CO(R^{y}), —CON(R^{xa})₂, —NHCON(R^{xa})₂, —NHCO—(C₁-C₄ alkyl), —NHCO₂—(C₁-C₄ alkyl), —NHSO₂—(C₁-C₄ alkyl), —OCH₂-aryl, —SO₂—(C₁-C₄ alkyl), —SO₂—N(R^{xa})₂, —SO₂-heterocyclyl, —N(R^{xa})₂, and nitro;
p is from 0 to 5;
R^{xa} and R^{xb} are independently selected from hydrogen, alkyl, or haloalkyl;
R^{y} is selected from C₁-C₂ dialkylamine or nitrogen-containing heterocyclyl, and is attached to the parent fragment through its nitrogen;
X and X¹ are each are independently a bond or are selected from:

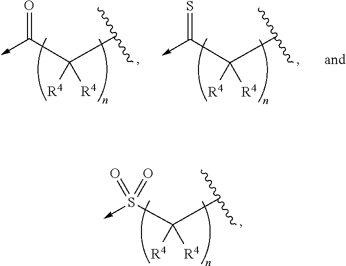

wherein the attachment of X, X¹ to the parent structure is such that the bond with the arrow is oriented toward the respective nitrogen shown in Formula II; provided, however, that when A is a bond, at least one X or X¹ is not a bond;
each n is independently from 0 to 2;
each R⁴ is independently selected from hydrogen, C₁-C₃ alkyl, C₂-C₃ alkenyl, aryl, aryl(C₁-C₂ alkyl), hydroxyl, halogen with the option for two R⁴s on same or adjacent carbon(s) to form a ring;
G and G′ are each independently selected from

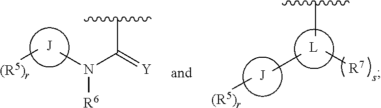

each Y is independently oxygen or sulfur;
each J is independently a bond or selected from aryl, heterocyclyl, or C₃-C₇ cycloalkyl;
each R⁵ is independently selected from hydrogen, C₁-C₄ alkoxy, C₂-C₄ (alkoxyalkyl), C₁-C₄ alkyl, halogen, C₂-C₅ bicycloalkyl, C₁-C₄ haloalkoxy, C₁-C₄ haloalkyl, —CONH₂, —CN, —NHCO(C₁-C₄ alkyl), —NHCON(C₁-C₄ alkyl)₂, —NHCO₂(C₁-C₄ alkyl), —OH, —SO₂N(C₁-C₄ alkyl)₂ and heterocyclyl;
each r is independently from 0 to 5;
each R⁶ is independently selected from hydrogen, C₁-C₄ alkyl, C₂-C₄ alkenyl, and C₃-C₄ cycloalkyl, optionally substituted with halogen, hydroxyl, C₁-C₂ alkoxy, or C₁-C₂ haloalkoxy;
each L is independently selected from a five or six-member heteroaryl ring;
each R⁷ is independently selected from C₁-C₃ alkoxy, C₁-C₃ alkyl, halogen, C₁-C₃ haloalkoxy, C₁-C₃ haloalkyl, —CONH₂, —CN, —OH, —C₂-C₅ alkynol, —NHCO(C₁-C₃ alkyl), —NHCON(C₁-C₃ alkyl)₂, —NHCO₂(C₁-C₃ alkyl), and —SO₂N(C₁-C₃alkyl)₂, and C₂-C₆ alkyne optionally substituted with 1 to 2 halides;
each s is independently from 0 to 4;
M and M′ are independently selected from C₁-C₇ alkyl, C₂-C₇ alkenyl, C₂-C₇ alkynyl, C₅-C₈ bicycloalkyl, C₃-C₇ cycloalkyl, aryl, and heterocyclyl;
each R^{3a} and R^{3b} is independently selected from C₂-C₄ alkenoxy, C₂-C₄ alkenyl, C₁-C₄ alkoxy, C₂-C₄ (alkoxyalkyl), (C₁-C₄alkoxy)carbonyl, C₁-C₄ alkyl, C₁-C₄ haloalkyl, C₁-C₄ haloalkoxy, carboxyamide, halogen, —CN, —NHCO(C₁-C₄ alkyl), —OH, C₁-C₄ hydroxyalkyl, and —SO₂N-heterocycle; and
q and q′ are each independently from 0 to 5;
wherein the attachment of “X”, “X¹” or N to “A” could be on the same or different atom(s) of “A”.

In an aspect of the invention, there is provided a compound of Formula III, including pharmaceutically acceptable salts thereof:

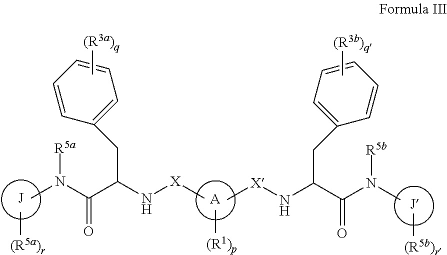

wherein
A is a bond or is selected from C₁-C₅ alkyl, C₂-C₅ alkenyl, C₂-C₅ alkynyl, aryl, C₃-C₆ cycloalkyl, C₂-C₅ bicycloalkyl, —CO—, —CS—, —C(═N—CN)—, heterocyclyl, nitrogen, sulfur, oxygen, —O—(C₂-C₄ alkyl)-O—, —N(R^{xa})CON(R^{xb})—, and ferrocene;
each R¹ is independently selected from hydrogen, C₁-C₄ alkyl, C₂-C₄ alkenyl, C₁-C₄ alkoxy, C₂-C₄ (alkoxyalkyl), (C₁-C₄ alkoxy)carbonyl, C₁-C₄ alkylthioxy, benzyloxy, C₂-C₄ alkynyl, aryl, carboxylic acid, cyano, halogen, C₁-C₄ haloalkyl, C₁-C₄ haloalkoxy, heterocyclyl, hydroxy, C₁-C₄ hydroxyalkyl, thioxy, —CH₂NH₂, —(C₁-C₄ alkyl)-heteroaryl, —CO—(C₁-C₄ alkyl), —CO(R^{y}), —CON(R^{xa})₂, —NHCON(R^{xa})₂, —NHCO—(C₁-C₄ alkyl), —NHCO₂—(C₁-C₄ alkyl), —NHSO₂—(C₁-C₄ alkyl), —OCH₂-aryl, —SO₂—(C₁-C₄ alkyl), —SO₂—N(R^{xa})₂, —SO₂-heterocyclyl, —N(R^{xa})₂, and nitro;
p is from 0 to 5;
R^{xa} and R^{xb} are independently selected from hydrogen, alkyl, or haloalkyl;
R^{y} is selected from C₁-C₂ dialkylamine or a nitrogen-containing heterocyclyl, and is attached to the parent fragment through its nitrogen;
X and X¹ are each are independently a bond or are selected from:

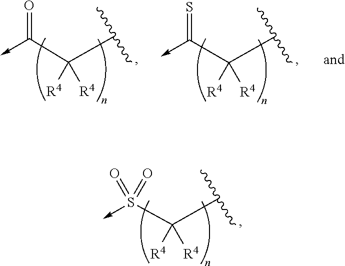

wherein the attachment of X, X¹ to the parent structure is such that the bond with the arrow is oriented toward the respective nitrogen shown in Formula III; provided, however, that when A is a bond, at least one X or X¹ is not a bond;
each n is independently from 0 to 2;
each R⁴ is independently selected from C₁-C₃ alkyl, C₂-C₃ alkenyl, aryl, aryl(C₁-C₂ alkyl), hydroxyl, and halogen, with the option for two R⁴ on the same or adjacent carbon(s) to form a ring;
J and J′ are independently a bond or are independently selected from aryl, heterocyclyl, or C₃-C₇ cycloalkyl;
R^{5a} and R^{5b} are independently selected from hydrogen, C₁-C₄ alkoxy, C₁-C₄ alkyl, C₂-C₄ (alkoxyalkyl), C₃-C₄ cycloalkyl, halogen, C₁-C₄ haloalkoxy, C₁-C₄ haloalkyl, —CONH₂, —CN, —NHCO(C₁-C₄ alkyl), —NHCON(C₁-C₄ alkyl)₂, —NHCO₂(C₁-C₄ alkyl), —OH, —SO₂N(C₁-C₄ alkyl)₂ and heterocyclyl;
each r and r′ is independently from 0 to 4;
R^{6a} and R^{6b} are each independently selected from hydrogen, C₁-C₄ alkyl, C₂-C₄ alkenyl, and C₃-C₄ cycloalkyl, optionally substituted with halogen, hydroxyl, C₁-C₂ alkoxy, or C₁-C₂ haloalkoxy;
each R^{3a} and R^{3b} is independently selected from C₂-C₄ alkenoxy, C₂-C₄ alkenyl, C₁-C₄ alkoxy, C₂-C₄ (alkoxyalkyl), (C₁-C₄ alkoxy)carbonyl, C₁-C₄ alkyl, C₁-C₄ haloalkyl, C₁-C₄ haloalkoxy, carboxyamide, halogen, —CN, —NHCO(C₁-C₄ alkyl), —OH, C₁-C₄ hydroxyalkyl, and —SO₂N-heterocycle; and
q and q′ are independently from 0 to 4;
wherein the attachment of “X”, “X¹” or N to “A” could be on the same or different atom(s) of “A”.

In an aspect of the invention, there is provided a compound of Formula III, wherein A is selected from C₁-C₅ alkyl, C₂-C₅ alkenyl, aryl with 1 to 2 rings, C₃-C₆ cycloalkyl, —CO—, heterocyclyl with 1 to 2 rings, nitrogen, sulfur, oxygen, —O—(C₂-C₄ alkyl)-O—, —N(R^{xa})CON(R^{xb})—, and ferrocene;
each R¹ is independently selected from hydrogen, C₁-C₄ alkyl, C₂-C₃ alkenyl, C₁-C₂ alkoxy, aryl, carboxylic acid, cyano, halogen, C₁-C₂ haloalkyl, C₁-C₂ haloalkoxy, heterocyclyl, hydroxy, C₁-C₄ hydroxyalkyl, —CO—(C₁-C₄ alkyl), CO(R^{y}), —CON(R^{xa})₂, —NHCON(R^{xa})₂, —SO₂—(C₁-C₄ alkyl), —SO₂—N(R^{xa})₂, —SO₂-heterocyclyl, and —N(R^{xa})₂;
p is from 0 to 4;
each R⁴ is independently selected from hydrogen, C₁-C₃ alkyl, aryl(C₁-C₂ alkyl), hydroxyl, or halogen with the option for two “R⁴”s on the same or adjacent carbon(s) to form a ring; and
n is from 0 to 2.

In an aspect of the invention, there is provided a compound of Formula III, wherein A is selected from C₁-C₅ alkyl, C₂-C₅ alkenyl, aryl with 1 to 2 rings, C₃-C₆ cycloalkyl, —CO—, heterocyclyl with 1 to 2 rings, nitrogen, oxygen, —O—(C₂-C₄ alkyl)-O—, —N(R^{xa})CON(R^{xb})—, and ferrocene;
each R¹ is independently selected from the group of hydrogen, C₁-C₄ alkyl, C₂-C₃ alkenyl, C₁-C₂ alkoxy, aryl, carboxylic acid, cyano, halogen, C₁-C₂ haloalkyl, C₁-C₂ haloalkoxy, heterocyclyl, hydroxy, C₁-C₄ hydroxyalkyl, —CO—(C₁-C₄ alkyl), CO(R^{y}), —CON(R^{xa})₂, —NHCON(R^{xa})₂, —SO₂—(C₁-C₄ alkyl), —SO₂—N(R^{xa})₂, —SO₂-heterocyclyl, and —N(R^{xa})₂;
p is from 0 to 4;
each R⁴ is independently selected from hydrogen, C₁-C₃ alkyl, aryl(C₁-C₂ alkyl), hydroxyl, or halogen with the option for two “R⁴”s on the same or adjacent carbon(s) to form a ring; and
n is from 0 to 2.

In an aspect of the invention, there is provided a compound of Formula III, wherein each R^{3a} and R^{3b} is independently selected from C₂-C₄ alkenoxy, C₂-C₄ alkenyl, C₁-C₄ alkoxy, C₁-C₄ alkyl, C₁-C₄ haloalkyl, C₁-C₄ haloalkoxy, halogen, —CN, and —OH;
q and q′ are independently from 0 to 3;
J and J′ are independently selected from 1-2 ring aryl, and 1-2 ring heteroaryl;
R^{5a} and R^{5b} are independently selected from hydrogen, C₁-C₄ alkoxy, C₁-C₄ alkyl, C₃-C₄ cycloalkyl, halogen, C₁-C₄ haloalkoxy, C₁-C₄ haloalkyl, C₃-C₄ cycloalkyl, —CONH₂, —CN, —NHCO(C₁-C₂ alkyl), —NHCON(C₁-C₂alkyl)₂, —NHCO₂(C₁-C₂ alkyl), —OH, and heterocyclyl;
r and r′ are independently from 0 to 4; and
R^{6a} and R^{6b} are independently selected from hydrogen, C₁-C₄ alkyl, C₁-C₄ alkenyl, or C₃-C₄ cycloalkyl, and with the option for each to be substituted with halogen.

In an aspect of the invention, there is provided a compound of Formula III, wherein each R^{3a} and R^{3b} is independently selected from C₂-C₄ alkenyl, C₁-C₂ alkoxy, C₁-C₄ alkyl, C₁-C₃ haloalkyl, C₁-C₃ haloalkoxy, halogen, and —CN;
q and q′ are independently from 0 to 3;
J and J′ are independently selected from 1-2 ring aryl, and 1-2 ring heteroaryl;
R^{5a} and R^{5b} are independently selected from the group of C₁-C₄ alkoxy, C₁-C₄ alkyl, C₃-C₄ cycloalkyl, halogen, C₁-C₄ haloalkoxy, C₁-C₄ haloalkyl, C₃-C₄ cycloalkyl, —CONH₂, —CN, —NHCO(C₁-C₂ alkyl), —NHCON(C₁-C₂alkyl)₂, —NHCO₂(C₁-C₂ alkyl), —OH, and heterocyclyl;
r and r′ are independently from 0 to 4; and
R^{6a} and R^{6b} are independently selected from hydrogen, C₁-C₄ alkyl, C₁-C₄ alkenyl, or C₃-C₄ cycloalkyl, and with the option for each to be substituted with halogen.

In an aspect of the invention, there is provided a compound of Formula III, wherein A is selected from CO, nitrogen, sulfur, oxygen, (CH₂)ₜ where t=1-4, —CH═CH—, —CH═C(Me)CH₂—, —CH═CH—CH₂—, —OCH₂CH₂O—, —NH(CO)NH—, cyclopentyl, cyclohexyl, phenyl, biphenyl, pyridine, pyrimidine, bipyrimidine, pyridazine, pyrazine, triazine, piperizine, pyrazole, thiophene, imidazole, isoxazole, indole, 1,3-dihydrobenzo[c][1,2,5]thiadiazole 2,2-dioxide, 1H-benzo[d]imidazol-2(3H)-one, imidazolidin-2-one, 2,3-dihydrophthalazine-1,4-dione, quinoxaline-2,3(1H,4H)-dione, 3-hydroxyquinoxalin-2(1H)-one, quinazoline-2,4(1H,3H)-dione, and ferrocene;
each R¹ is independently selected from H, C₁-C₄ alkyl, C₁-C₂ haloalkyl, C₁-C₂ alkoxy, C₁-C₂ haloalkoxy, C₁-C₄ hydroxyalkyl, OH, CO₂H, cyano, halogen, C₁-C₂ haloalkoxy, amine, and acetamide;
p is from 0 to 4;
R⁴ is selected from hydrogen, C₁-C₂ alkyl, or benzyl; and
n is from 0 to 2.

In an aspect of the invention, there is provided a compound of Formula III, wherein each R^{3a} and R^{3b} is independently selected from halogen, C₁-C₂ alkyl, C₁-C₂ haloalkyl, C₁-C₂ alkoxy, and C₁-C₂ haloalkoxy;
q and q′ are independently from 0 to 2;
J and J′ are each independently selected from phenyl, pyridine, pyrimidine, pyrazine, pyridazine, benzothiazole, benzothiazolone, benzothiadiazole, benzodioxole, benzoxazolone, benzisothiazole, 1-methylpyridin-2(1H)-one, 2,3-dihydrobenzo[b][1,4]dioxine, indazole, benzimidazole, and quinoxaline;
R^{5a} and R^{5b} are each selected from hydrogen, C₁-C₄ alkyl, C₃-C₄ cycloalkyl, C₁-C₂ alkoxy, C₁-C₂ haloalkoxy, C₁-C₂ haloalkyl, methylcarbamate, benzyl, morpholinyl, halide, and CN;
r and r′ are independently selected from 0 to 2; and
R^{6a} and R^{6b} are independently selected from hydrogen, C₁-C₄ alkyl, C₁-C₄ haloalkyl, and C₁-C₄ alkenyl.

In an aspect of the invention, there is provided a compound of Formula IV, including pharmaceutically acceptable salts thereof:

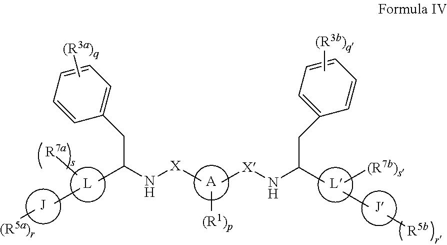

wherein
A is a bond or is selected from C₁-C₅ alkyl, C₂-C₅ alkenyl, C₂-C₅ alkynyl, aryl, C₃-C₆ cycloalkyl, —C₂-C₅ bicycloalkyl, —CO—, —CS—, —C(═N—CN)—, heterocyclyl, nitrogen, sulfur, oxygen, —O—(C₂-C₄ alkyl)-O—, —N(R^{xa})CON(R^{xb})—, and ferrocene;
each R¹ is independently selected from hydrogen, C₁-C₄ alkyl, C₂-C₄ alkenyl, C₁-C₄ alkoxy, C₂-C₄ (alkoxyalkyl), (C₁-C₄ alkoxy)carbonyl, C₁-C₄ alkylthioxy, benzyloxy, C₂-C₄ alkynyl, aryl, carboxylic acid, cyano, halogen, C₁-C₄ haloalkyl, C₁-C₄ haloalkoxy, heterocyclyl, hydroxy, C₁-C₄ hydroxyalkyl, thioxy, —CH₂NH₂, —(C₁-C₄ alkyl)-heteroaryl, —CO—(C₁-C₄ alkyl), —CO(R^{y}), —CON(R^{xa})₂, —NHCON(R^{xa})₂, —NHCO—(C₁-C₄ alkyl), —NHCO₂—(C₁-C₄ alkyl), —NHSO₂—(C₁-C₄ alkyl), —OCH₂-aryl, —SO₂—(C₁-C₄ alkyl), —SO₂—N(R^{xa})₂, —SO₂-heterocyclyl, —N(R^{xa})₂, and nitro;
p is from 0 to 5;
R^{xa} and R^{xb} are independently selected from hydrogen, alkyl, or haloalkyl;
R^{y} is selected from C₁-C₂ dialkylamine or a nitrogen-containing heterocyclyl and is attached to the parent fragment through its nitrogen.
X and X¹ are each are independently a bond or are selected from:

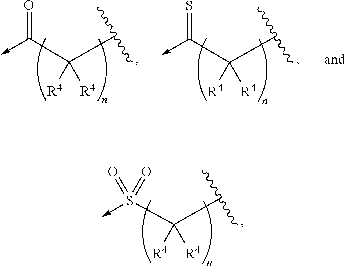

wherein the attachment of X, X¹ to the parent structure is such that the bond with the arrow is oriented toward the respective nitrogen shown in Formula IV; provided, however, that when A is a bond, at least one X or X¹ is not a bond;
each n is independently from 0 to 2;
each R⁴ is independently selected from C₁-C₃ alkyl, C₂-C₃ alkenyl, aryl, aryl(C₁-C₂ alkyl)-, hydroxyl, and halogen, with the option for two R⁴ on same or adjacent carbon(s) to form a ring;
J and J′ are independently a bond or selected from aryl, heterocyclyl, or C₃-C₇ cycloalkyl;
each R^{5a} and R^{5b} is independently selected from hydrogen, C₁-C₄ alkoxy, C₁-C₄ alkyl, C₂-C₄ (alkoxyalkyl), C₃-C₄ cycloalkyl, halogen, C₁-C₄ cycloalkyl, C₁-C₄ haloalkoxy, C₁-C₄ haloalkyl, —CONH₂, —CN, —NHCO(C₁-C₄ alkyl), —NHCON(C₁-C₄ alkyl)₂, —NHCO₂(C₁-C₄ alkyl), —OH, —SO₂N(C₁-C₄ alkyl)₂ and heterocyclyl;
r and r′ are independently from 0 to 4;
L and L′ are independently selected from a five or six-member heteroaryl ring;
each R^{7a} and R^{7b} is independently selected from C₁-C₃ alkoxy, C₁-C₃ alkyl, halogen, C₁-C₃ haloalkoxy, C₁-C₃ haloalkyl, —CONH₂, —CN, —OH, C₂-C₅ alkynol, —NHCO(C₁-C₃ alkyl), —NHCON(C₁-C₃ alkyl)₂, —NHCO₂(C₁-C₃ alkyl), and —SO₂N(C₁-C₃ alkyl)₂, and C₂-C₆ alkyne optionally substituted with 1 to 2 halides;
s and s′ are independently from 0 to 4;
each R^{3a} and R^{3b} is independently selected from C₂-C₄ alkenoxy, C₂-C₄ alkenyl, C₁-C₄ alkoxy, C₂-C₄ (alkoxyalkyl), (C₁-C₄alkoxy)carbonyl, C₁-C₄ alkyl, C₁-C₄ haloalkyl, C₁-C₄ haloalkoxy, carboxyamide, halogen, —CN, —NHCO(C₁-C₄ alkyl), —OH, C₁-C₄ hydroxyalkyl, and —SO₂N-heterocycle; and
q and q′ are independently from 0 to 4;
wherein the attachment of “X”, “X¹” or N to “A” could be on the same or different atom(s) of “A”.

In an aspect of the invention, there is provided a compound of Formula IV, wherein A is selected from C₁-C₅ alkyl, C₂-C₅ alkenyl, aryl with 1 to 2 rings, C₃-C₆ cycloalkyl, —CO—, heterocyclyl with 1 to 2 rings, nitrogen, sulfur, oxygen, —O—(C₂-C₄ alkyl)-O—, —N(R^{xa})CON(R^{xb})—, and ferrocene;
each R¹ is independently selected from the group of hydrogen, C₁-C₄ alkyl, C₂-C₃ alkenyl, C₁-C₂ alkoxy, aryl, carboxylic acid, cyano, halogen, C₁-C₂ haloalkyl, C₁-C₂ haloalkoxy, heterocyclyl, hydroxy, C₁-C₄ hydroxyalkyl, —CO—(C₁-C₄ alkyl), CO(R^{y}), —CON(R^{xa})₂, —NHCON(R^{xa})₂, —SO₂—(C₁-C₄ alkyl), —SO₂—N(R^{xa})₂, —SO₂-heterocyclyl, and —N(R^{xa})₂;
p is from 0 to 4;
each R⁴ is independently selected from hydrogen, C₁-C₃ alkyl, aryl(C₁-C₂ alkyl), hydroxyl, and halogen, with the option for two R⁴ on the same or adjacent carbon(s) to form a ring;
n is from 0 to 2; and
each R^{3a} and R^{3b} is independently selected from C₂-C₄ alkenoxy, C₂-C₄ alkenyl, C₁-C₄ alkoxy, C₁-C₄ alkyl, C₁-C₄ haloalkyl, C₁-C₄ haloalkoxy, halogen, —CN, and —OH; and
q and q′ are independently selected from 0 to 3.

In an aspect of the invention, there is provided a compound of Formula IV, wherein J and J′ are independently selected from 1-2 ring aryl, and 1-2 ring heteroaryl;
R^{5a} and R^{5b} are independently selected from hydrogen, C₁-C₄ alkoxy, C₁-C₄ alkyl, C₃-C₄ cycloalkyl, halogen, C₁-C₄ haloalkoxy, C₁-C₄ haloalkyl, C₃-C₄ cycloalkyl, —CONH₂, —CN, —NHCO(C₁-C₂ alkyl), —NHCON(C₁-C₂ alkyl)₂, —NHCO₂(C₁-C₂ alkyl), —OH, and heterocyclyl;
r and r′ are independently from 0 to 4;
L and L′ are independently selected from a five or six-member heteroaryl ring containing at least one nitrogen atom and that the moiety is attached to the central parental structure through an adjacent carbon atom; and
R^{7a} and R^{7b} are each independently selected from C₁-C₃ alkoxy, C₁-C₃ alkyl, halogen, C₁-C₃ haloalkoxy, C₁-C₃ haloalkyl, —CONH₂, —CN, OH, C₂-C₅ alkyne that is optionally substituted with 1 to 2 halide, C₂-C₅ alkynol, —NHCO(C₁-C₃ alkyl), —NHCON(C₁-C₃ alkyl)₂, —NHCO₂(C₁-C₃ alkyl), and —SO₂N(C₁-C₃ alkyl)₂.

In an aspect of the invention, there is provided a compound of Formula IV, wherein A is selected from CO, nitrogen, sulfur, oxygen, (CH₂)ₜ where t=1-4, —CH═CH—, —CH═C(Me)CH₂—, —CH═CH—CH₂—, —OCH₂CH₂O—, —NH(CO)NH—, cyclopentyl, cyclohexyl, phenyl, biphenyl, pyridine, pyrimidine, bipyrimidine, pyridazine, pyrazine, triazine, piperizine, pyrazole, thiophene, imidazole, isoxazole, indole, 1,3-dihydrobenzo[c][1,2,5]thiadiazole 2,2-dioxide, 1H-benzo[d]imidazol-2(3H)-one, imidazolidin-2-one, 2,3-dihydrophthalazine-1,4-dione, quinoxaline-2,3(1H,4H)-dione, 3-hydroxyquinoxalin-2(1H)-one, quinazoline-2,4(1H,3H)-dione, and ferrocene;
each R¹ is independently selected from H, C₁-C₄ alkyl, C₁-C₂ haloalkyl, C₁-C₂ alkoxy, C₁-C₂ haloalkoxy, C₁-C₄ hydroxyalkyl, OH, CO₂H, cyano, halogen, C₁-C₂ haloalkoxy, amine, and acetamide;
p is from 0 to 4;
R⁴ is selected from hydrogen, C₁-C₂ alkyl, or benzyl;
n is from 0 to 2;
each R^{3a} and R^{3b} is independently selected from halogen, C₁-C₂ alkyl, C₁-C₂ haloalkyl, C₁-C₂ alkoxy, and C₁-C₂ haloalkoxy; and
q and q′ are each independently from 0 to 2.

In an aspect of the invention, there is provided a compound of Formula IV, wherein J and J′ are each independently selected from phenyl, pyridine, pyrimidine, pyrazine, pyridazine, benzothiazole, benzothiazolone, benzothiadiazole, benzodioxole, benzoxazolone, benzisothiazole, 1-methylpyridin-2(1H)-one, 2,3-dihydrobenzo[b][1,4]dioxine, indazole, benzimidazole, and quinoxaline;
R^{5a} and R^{5b} are each independently selected from hydrogen, C₁-C₄ alkyl, C₃-C₄ cycloalkyl, C₁-C₂ alkoxy, C₁-C₂ haloalkoxy, C₁-C₂ haloalkyl, methylcarbamate, benzyl, morpholinyl, halide, and CN;
r and r′ are independently from 0 to 2; and
L and L′ are independently selected from a pyridine or an imidazole ring that is attached to the central parental structure through an adjacent carbon atom; and
each of R^{7a} and R^{7b} is independently selected from hydrogen, a C₂-C₅ alkyne that is optionally substituted with 1 to 2 halide, or a C₂-C₅ alkynol.

In an aspect of the invention, there is provided a compound of Formula V, including pharmaceutically acceptable salts thereof:

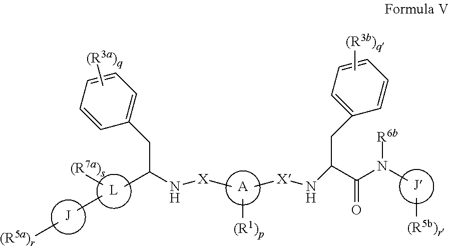

wherein
A is a bond or is selected from C₁-C₅ alkyl, C₂-C₅ alkenyl, C₂-C₅ alkynyl, aryl, C₃-C₆ cycloalkyl, —C₂-C₅ bicycloalkyl, —CO—, —CS—, —C(═N—CN)—, heterocyclyl, nitrogen, sulfur, oxygen, —O—(C₂-C₄ alkyl)-O—, —N(R^{xa})CON(R^{xb})—, and ferrocene;
each R¹ is independently selected from hydrogen, C₁-C₄ alkyl, C₂-C₄ alkenyl, C₁-C₄ alkoxy, C₂-C₄ (alkoxyalkyl), (C₁-C₄ alkoxy)carbonyl, C₁-C₄ alkylthioxy, benzyloxy, C₂-C₄ alkynyl, aryl, carboxylic acid, cyano, halogen, C₁-C₄ haloalkyl, C₁-C₄ haloalkoxy, heterocyclyl, hydroxy, C₁-C₄ hydroxyalkyl, thioxy, —CH₂NH₂, —(C₁-C₄ alkyl)-heteroaryl, —CO—(C₁-C₄ alkyl), —CO(R^{y}), —CON(R^{xa})₂, —NHCON(R^{xa})₂, —NHCO—(C₁-C₄ alkyl), —NHCO₂—(C₁-C₄ alkyl), —NHSO₂—(C₁-C₄ alkyl), —OCH₂-aryl, —SO₂—(C₁-C₄ alkyl), —SO₂—N(R^{xa})₂, —SO₂-heterocyclyl, —N(R^{xa})₂, and nitro;
p is from 0 to 5;
R^{xa} and R^{xb} are independently selected from hydrogen, alkyl, or haloalkyl;
R^{y} is selected from C₁-C₂ dialkylamine or a nitrogen-containing heterocyclyl and is attached to the parent fragment through its nitrogen;
X and X¹ are each are independently a bond or are selected from:

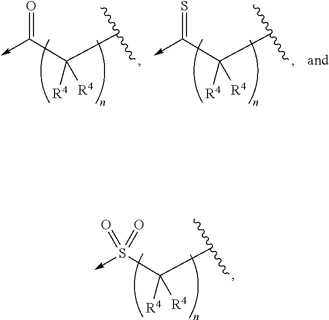

wherein the attachment of X and X¹ to the parent structure is such that the bond with the arrow is oriented toward the respective nitrogen shown in Formula V; provided, however, that when A is a bond, at least one X or X¹ is not a bond;
each n is independently from 0 to 2;
each R⁴ is independently selected from C₁-C₃ alkyl, C₂-C₃ alkenyl, aryl, aryl(C₁-C₂ alkyl)-, hydroxyl, and halogen, with the option for two R⁴ on same or adjacent carbon(s) to form a ring;
J and J′ are independently a bond or selected from aryl, heterocyclyl, or C₃-C₇ cycloalkyl;
R^{5a} and R^{5b} are independently selected from hydrogen, C₁-C₄ alkoxy, C₂-C₄ (alkoxyalkyl), C₁-C₄ alkyl, halogen, C₃-C₄ cycloalkyl, C₁-C₄ haloalkoxy, C₁-C₄ haloalkyl, —CONH₂, —CN, —NHCO(C₁-C₄ alkyl), —NHCON(C₁-C₄ alkyl)₂, —NHCO₂(C₁-C₄ alkyl), —OH, —SO₂N(C₁-C₄ alkyl)₂ and heterocyclyl;
r and r′ are independently from 0 to 4;
R^{6b} is selected from hydrogen, C₁-C₄ alkyl, C₁-C₄ alkenyl, and C₃-C₄ cycloalkyl, optionally substituted with halogen, hydroxyl, C₁-C₂ alkoxy, or C₁-C₂ haloalkoxy;
L is selected from a five or six-member heteroaryl ring;
R^{7a} is selected from C₁-C₃ alkoxy, C₁-C₃ alkyl, halogen, C₁-C₃ haloalkoxy, C₁-C₃ haloalkyl, —CONH₂, —CN, OH, C₂-C₅ alkynol, —NHCO(C₁-C₃ alkyl), —NHCON(C₁-C₃ alkyl)₂, —NHCO₂(C₁-C₃ alkyl), and —SO₂N(C₁-C₃ alkyl)₂, and C₂-C₆ alkyne optionally substituted with 1 to 2 halides;
each s is independently from 0 to 4;
each R^{3a} and R^{3b} is independently selected from C₂-C₄ alkenoxy, C₂-C₄ alkenyl, C₁-C₄ alkoxy, C₂-C₄ (alkoxyalkyl), (C₁-C₄alkoxy)carbonyl, C₁-C₄ alkyl, C₁-C₄ haloalkyl, C₁-C₄ haloalkoxy, carboxyamide, halogen, —CN, —NHCO(C₁-C₄ alkyl), —OH, C₁-C₄ hydroxyalkyl, and —SO₂N-heterocycle; and
q and q′ are independently from 0 to 4;
wherein the attachment of “X”, “X¹” or N to “A” could be on the same or different atom(s) of “A”.

In an aspect of the invention, there is provided a compound of Formula VI, including pharmaceutically acceptable salts thereof:

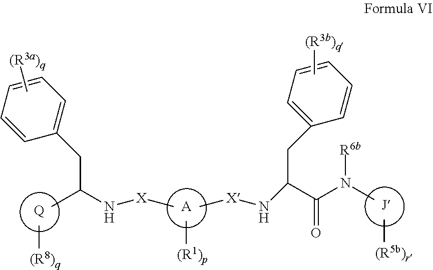

wherein
A is a bond or is selected from C₁-C₅ alkyl, C₂-C₅ alkenyl, C₂-C₅ alkynyl, aryl, C₃-C₆ cycloalkyl, —C₂-C₅ bicycloalkyl, —CO—, —CS—, —C(═N—CN)—, heterocyclyl, nitrogen, sulfur, oxygen, —O—(C₂-C₄ —N(R^{xa})CON(R^{xb})—, and ferrocene;
each R¹ is independently selected from hydrogen, C₁-C₄ alkyl, C₂-C₄ alkenyl, C₁-C₄ alkoxy, C₂-C₄ (alkoxyalkyl), (C₁-C₄ alkoxy)carbonyl, C₁-C₄ alkylthioxy, benzyloxy, C₂-C₄ alkynyl, aryl, carboxylic acid, cyano, halogen, C₁-C₄ haloalkyl, C₁-C₄ haloalkoxy, heterocyclyl, hydroxy, C₁-C₄ hydroxyalkyl, thioxy, —CH₂NH₂, —(C₁-C₄ alkyl)-heteroaryl, —CO—(C₁-C₄ alkyl), —CO(R^{y}), —CON(R^{xa})₂, —NHCON(R^{xa})₂, —NHCO—(C₁-C₄ alkyl), —NHCO₂—(C₁-C₄ alkyl), —NHSO₂—(C₁-C₄ alkyl), —OCH₂-aryl, —SO₂—(C₁-C₄ alkyl), —SO₂—N(R^{xa})₂, —SO₂-heterocyclyl, —N(R^{xa})₂, and nitro;
p is from 0 to 5;
R^{xa} and R^{xb} are independently selected from hydrogen, alkyl, or haloalkyl;
R^{y} is selected from C₁-C₂ dialkylamine or a nitrogen-containing heterocyclyl and is attached to the parent fragment through its nitrogen;
X and X¹ are each are independently a bond or are selected from:

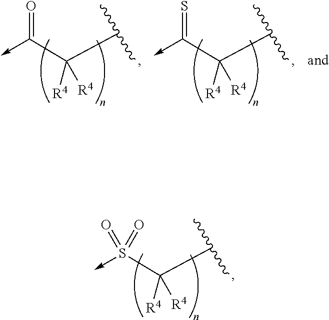

wherein the attachment of X and X¹ to the parent structure is such that the bond with the arrow is oriented toward the respective nitrogen shown in Formula VI; provided, however, that when A is a bond, at least one X or X¹ is not a bond;
each n is independently from 0 to 2;
each R⁴ is independently selected from C₁-C₃ alkyl, C₁-C₃ alkenyl, aryl, aryl(C₁-C₂ alkyl)-, hydroxyl, and halogen, with the option for two R⁴ on same or adjacent carbon(s) to form a ring;
J′ is a bond or is selected from aryl, heterocyclyl, or C₃-C₇ cycloalkyl;
R^{5b} is selected from hydrogen, C₁-C₄ alkoxy, C₁-C₄ alkyl, C₂-C₄ (alkoxyalkyl), C₃-C₄ cycloalkyl, halogen, C₁-C₄ haloalkoxy, C₁-C₄ haloalkyl, —CONH₂, —CN, —NHCO(C₁-C₄ alkyl), —NHCON(C₁-C₄ alkyl)₂, —NHCO₂(C₁-C₄ alkyl), —OH, —SO₂N(C₁-C₄ alkyl)₂ and heterocyclyl;
r′ is from 0 to 4;
R^{6b} is selected from hydrogen, C₁-C₄ alkyl, C₂-C₄ alkenyl, and C₃-C₄ cycloalkyl, optionally substituted with halogen, hydroxyl, C₁-C₂ alkoxy, or C₁-C₂ haloalkoxy;
Q is a bond or is selected from heterocycle and a —CON(C₁-C₃ alkyl)₂ with the option for the two alkyl groups together with the nitrogen atom to which they are attached to form a heterocycle;
R⁸ is selected from hydrogen, C₁-C₂ alkyl and C₁-C₂ alkyl-S—;
each R^{3a} and R^{3b} is independently selected from C₂-C₄ alkenoxy, C₂-C₄ alkenyl, C₁-C₄ alkoxy, C₂-C₄ (alkoxyalkyl), (C₁-C₄alkoxy)carbonyl, C₁-C₄ alkyl, C₁-C₄ haloalkyl, C₁-C₄ haloalkoxy, carboxyamide, halogen, —CN, —NHCO(C₁-C₄ alkyl), —OH, C₁-C₄ hydroxyalkyl, and —SO₂N-heterocycle; and
q and q′ are independently from 0 to 2;
wherein the attachment of “X”, “X¹” or N to “A” could be on the same or different atom(s) of “A”.

In an aspect of the invention, there is provided a compound of composition comprising a compound of the invention and a pharmaceutically acceptable carrier, excipient, and/or diluent.

In an aspect of the invention, there is provided a method of treating method of treating HIV infection comprising administering a therapeutically effective amount of a compound of the invention to a patient.

Preferred compounds of the invention, including pharmaceutically acceptable salts thereof, are selected from the group of:

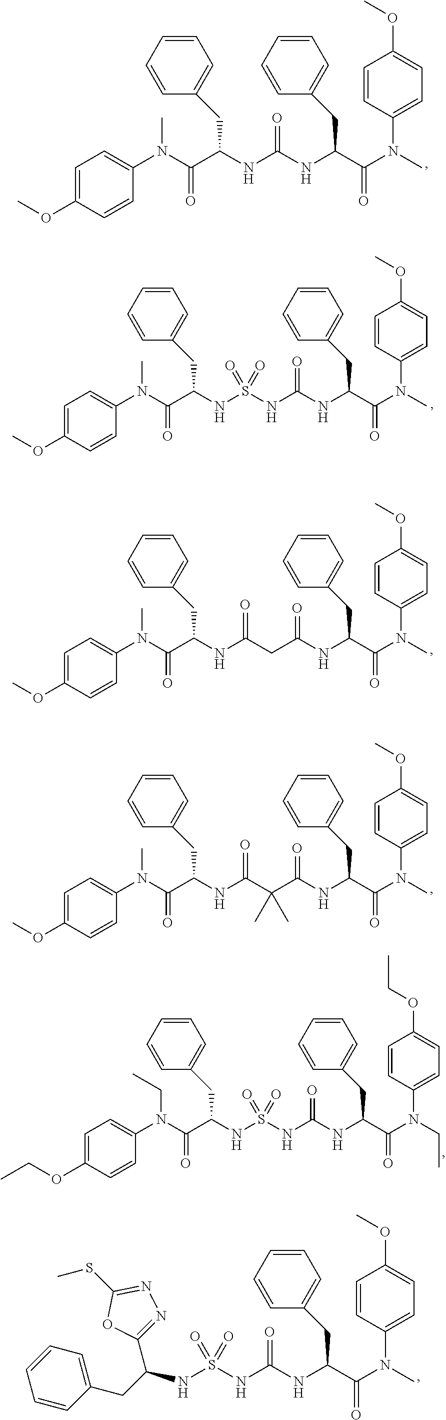

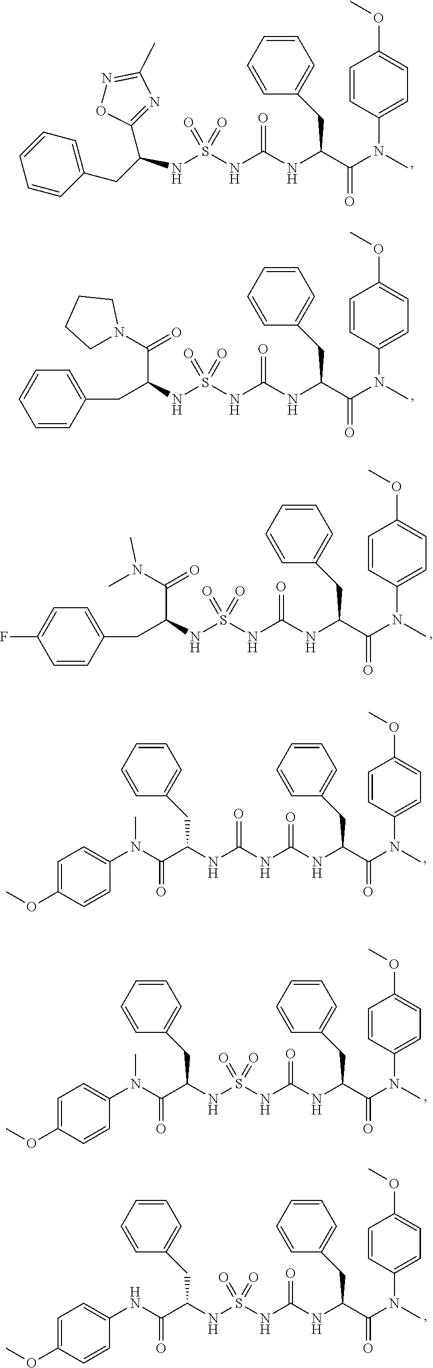

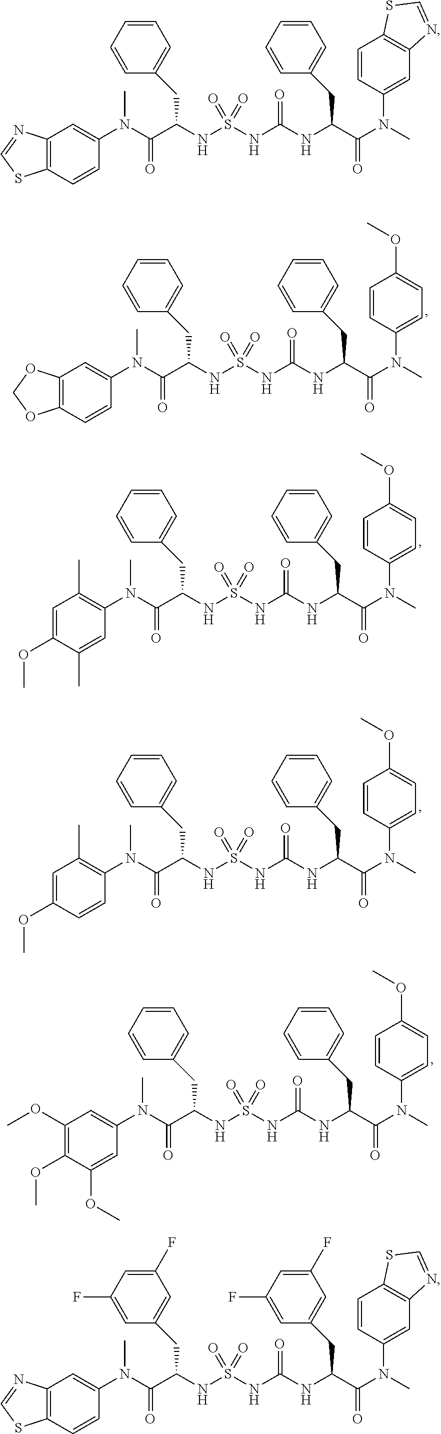

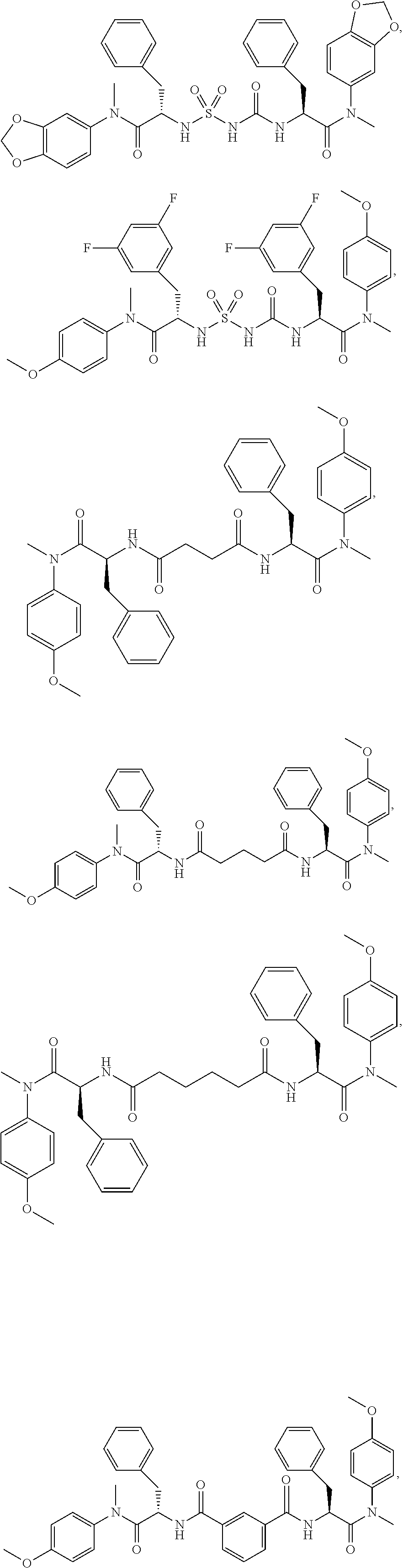

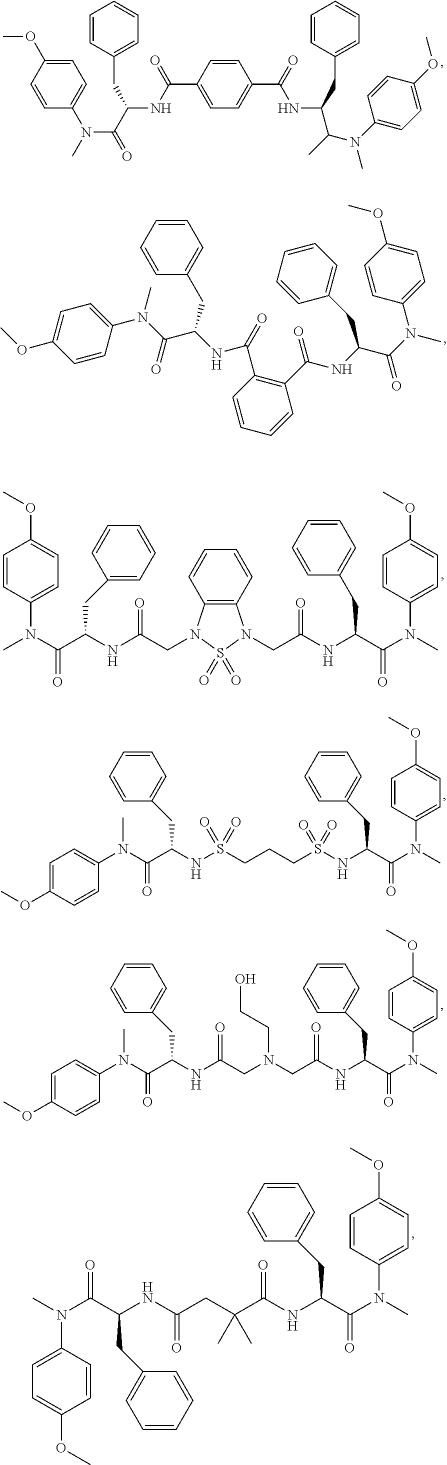

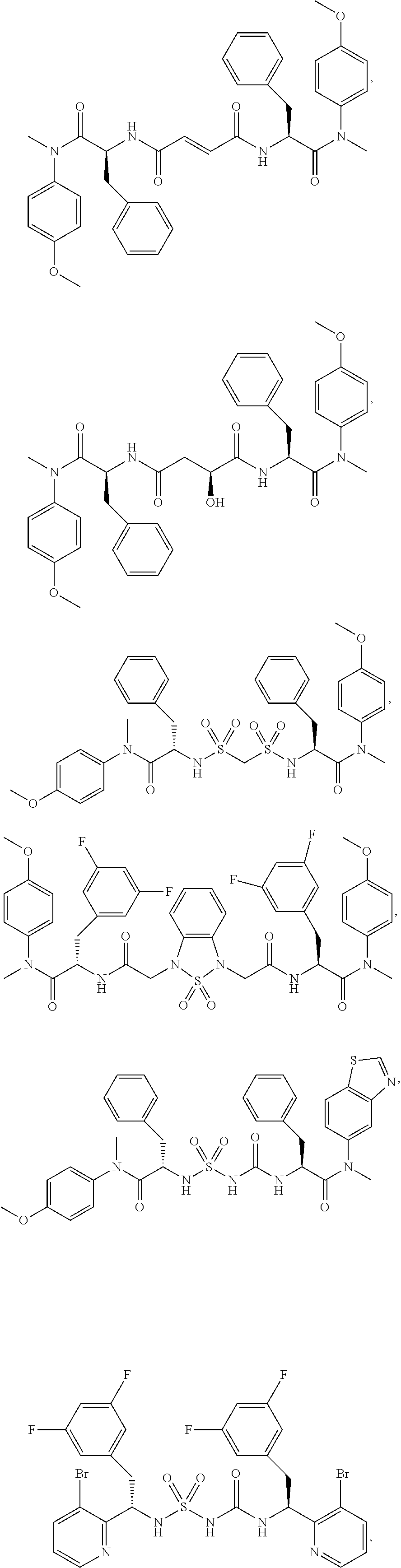

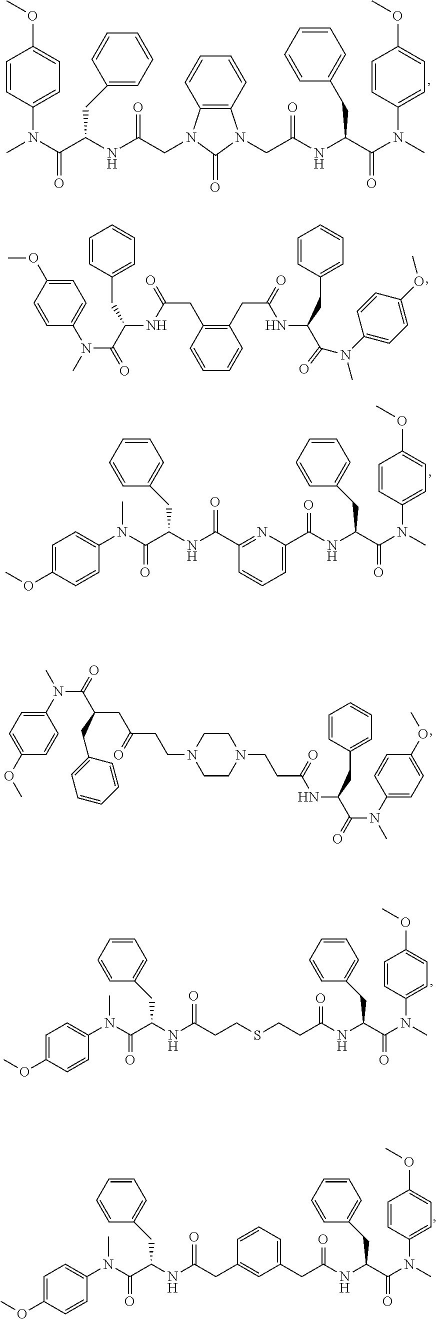

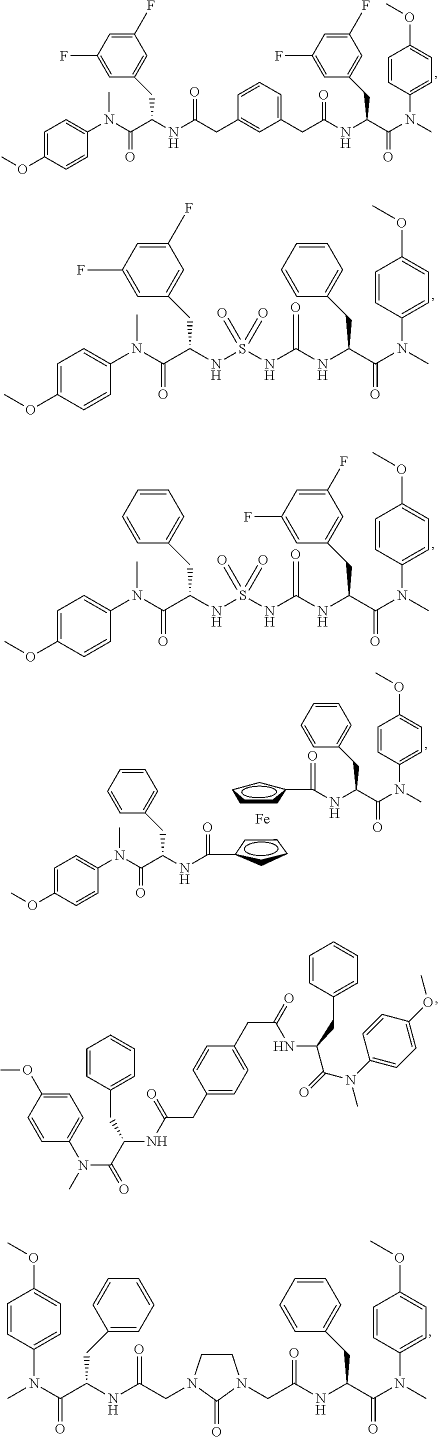

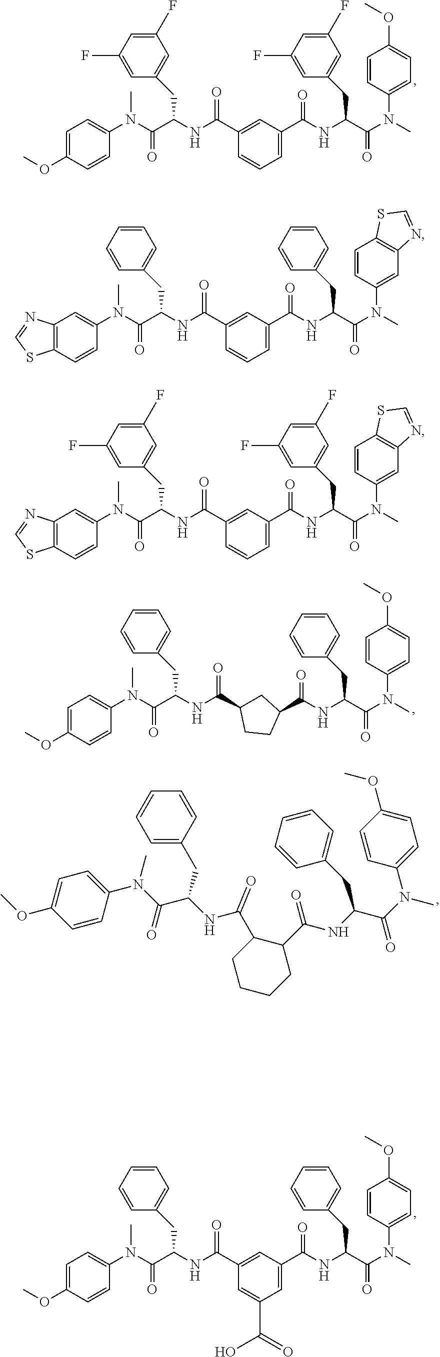

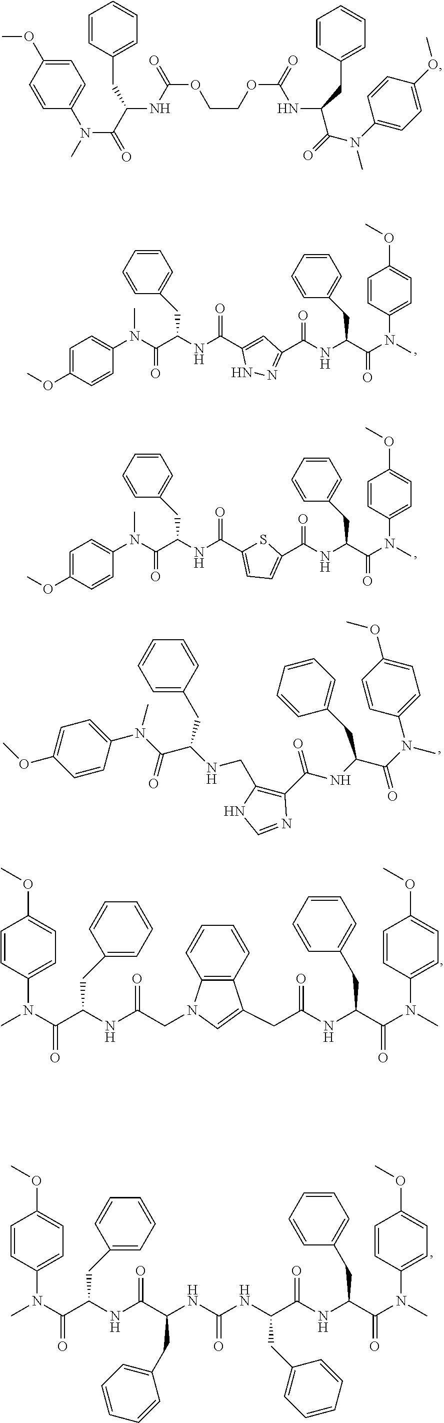

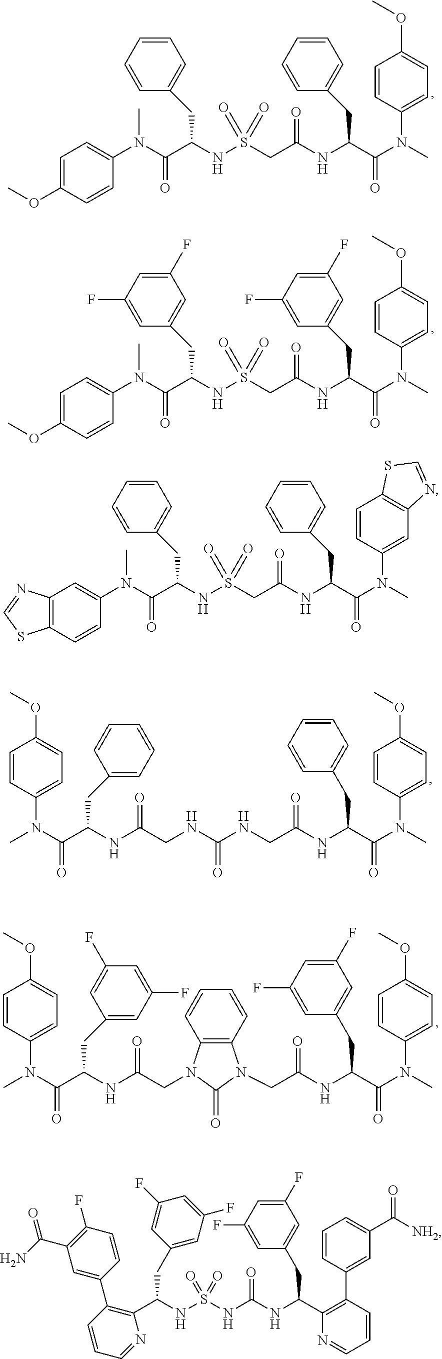

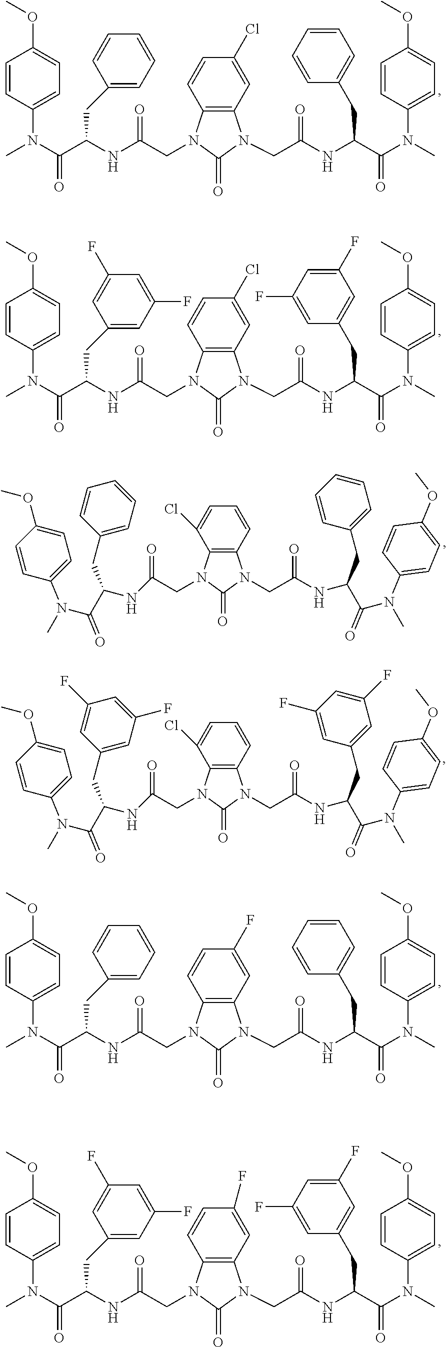

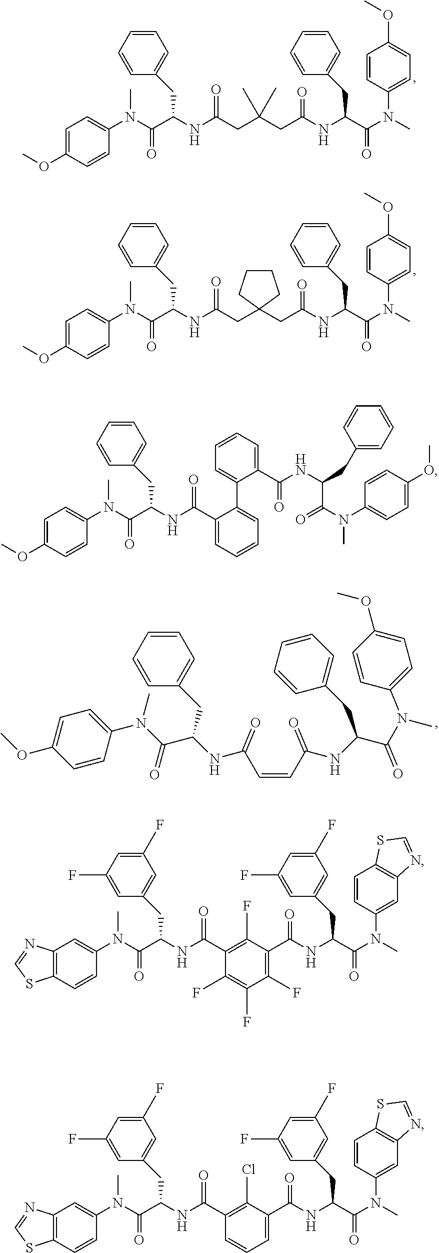

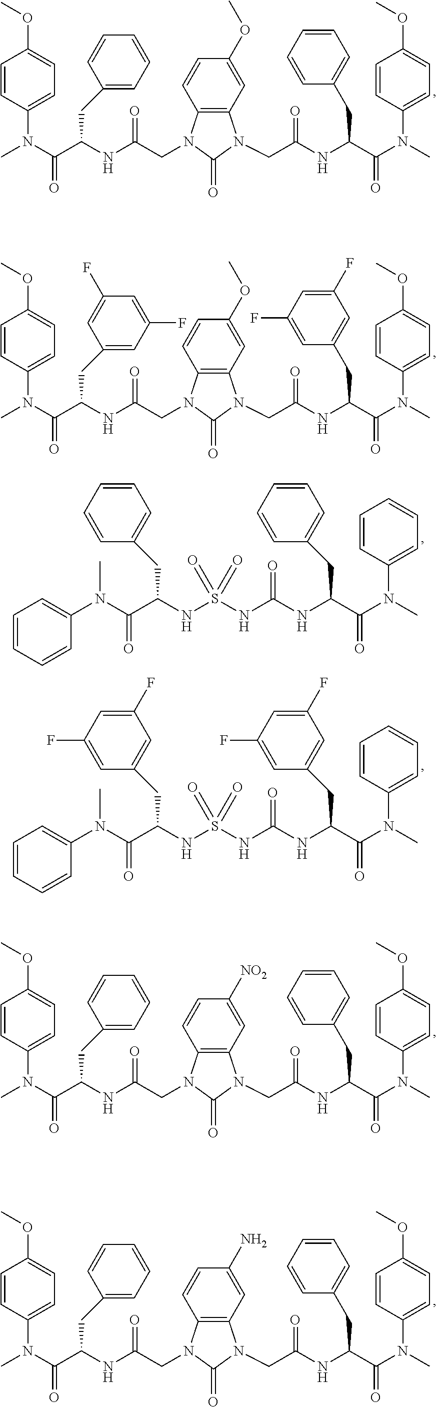

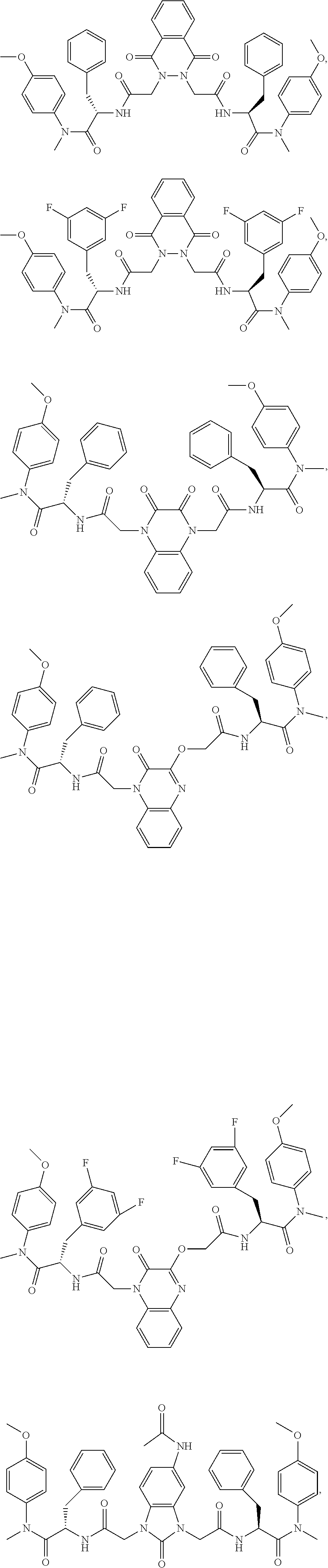

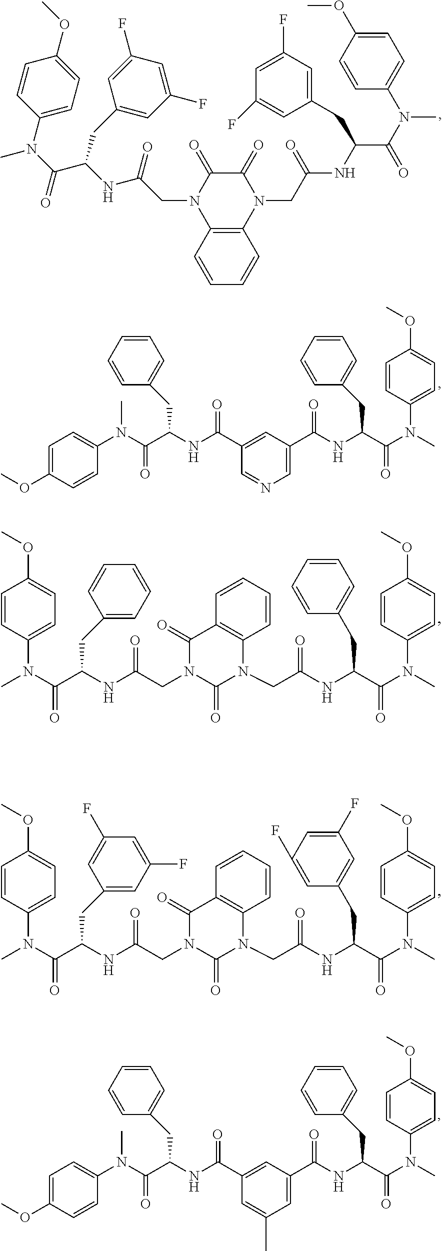

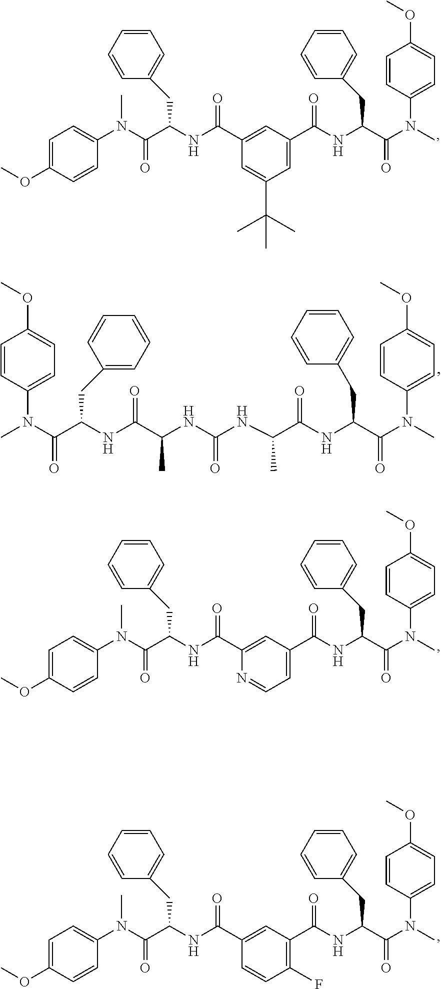 [SMILES: COc1ccc(N(C)C(=O)[C@H](Cc2ccccc2)NC(=O)c2cc(C(=O)N[C@@H](Cc3ccccc3)C(=O)N(C)c3ccc(OC)cc3)cc(C(C)(C)C)c2)cc1 ; COc1ccc(N(C)C(=O)[C@H](Cc2ccccc2)NC(=O)[C@H](C)NC(=O)N[C@@H](C)C(=O)N[C@@H](Cc2ccccc2)C(=O)N(C)c2ccc(OC)cc2)cc1 ; COc1ccc(N(C)C(=O)[C@H](Cc2ccccc2)NC(=O)c2ccnc(C(=O)N[C@@H](Cc3ccccc3)C(=O)N(C)c3ccc(OC)cc3)c2)cc1 ; COc1ccc(N(C)C(=O)[C@H](Cc2ccccc2)NC(=O)c2ccc(F)c(C(=O)N[C@@H](Cc3ccccc3)C(=O)N(C)c3ccc(OC)cc3)c2)cc1]

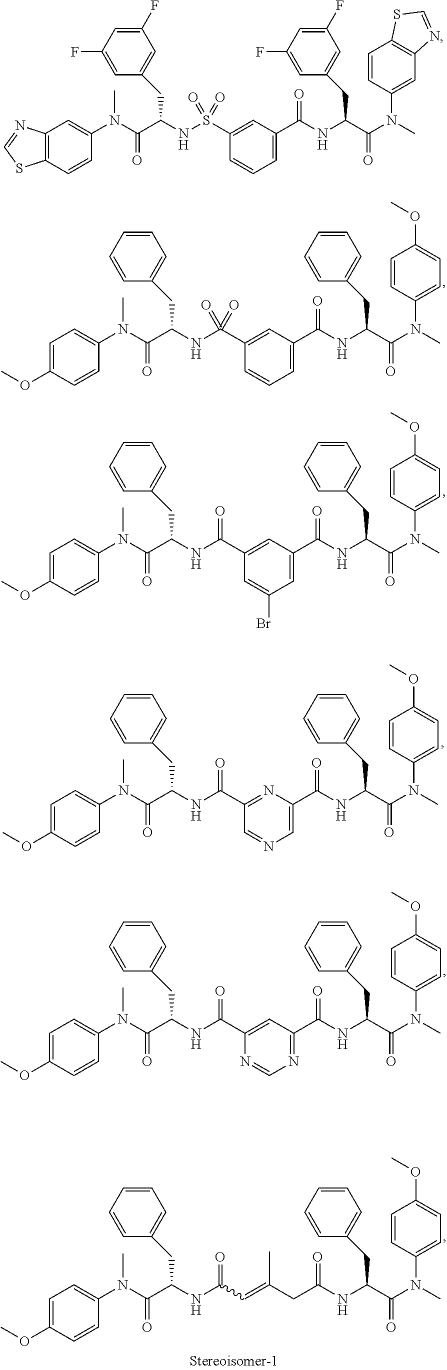

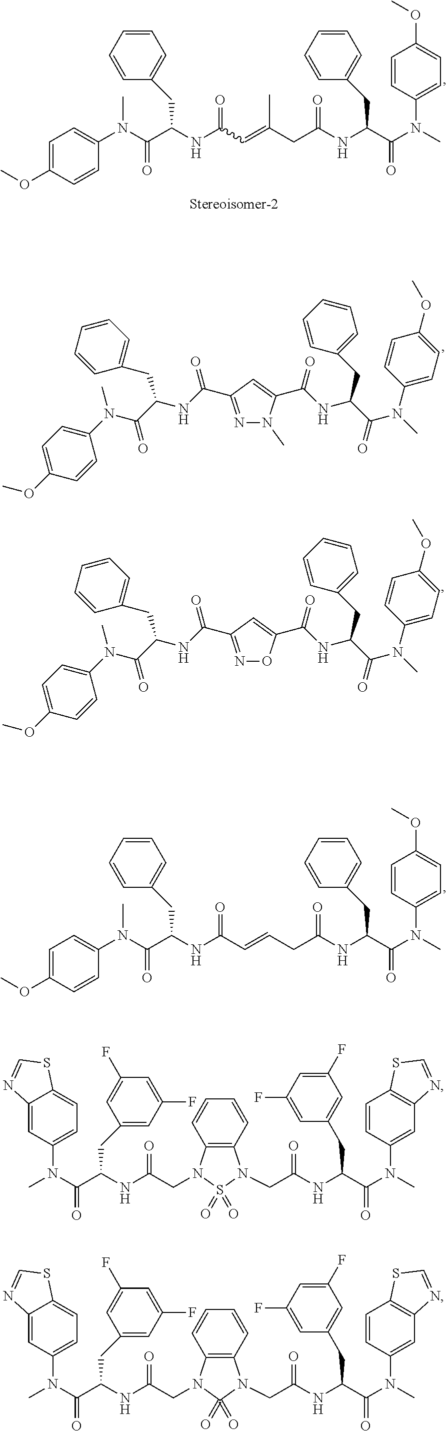

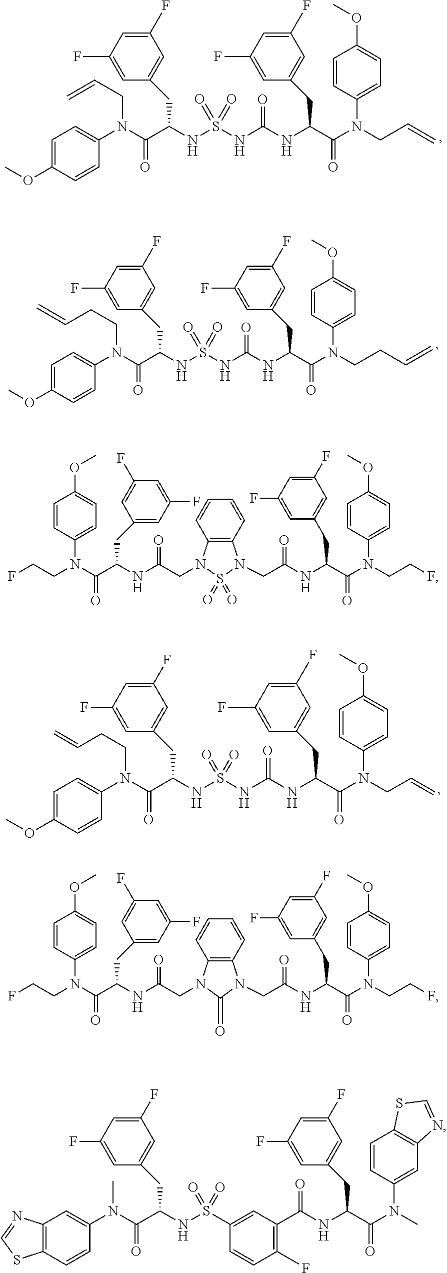

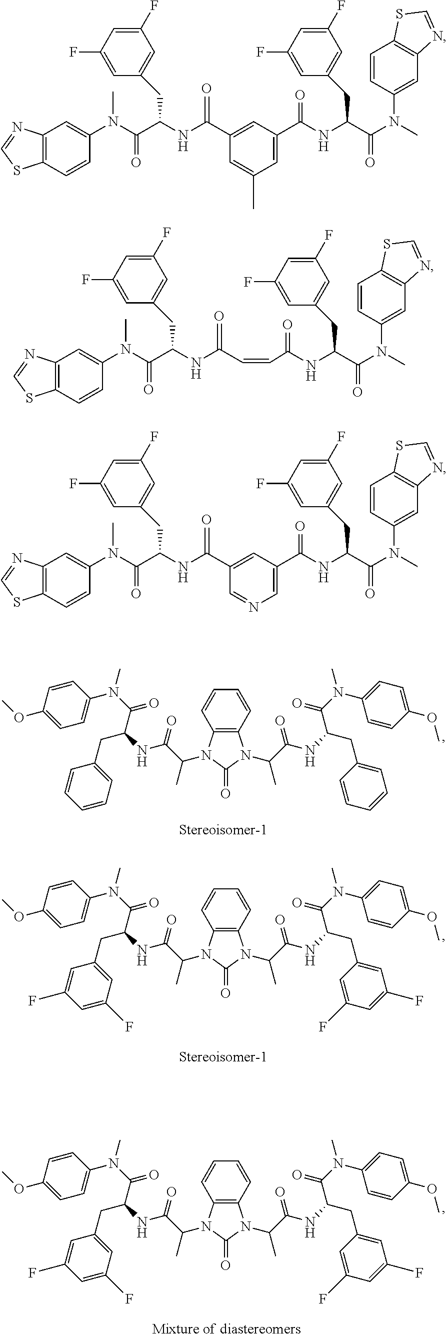

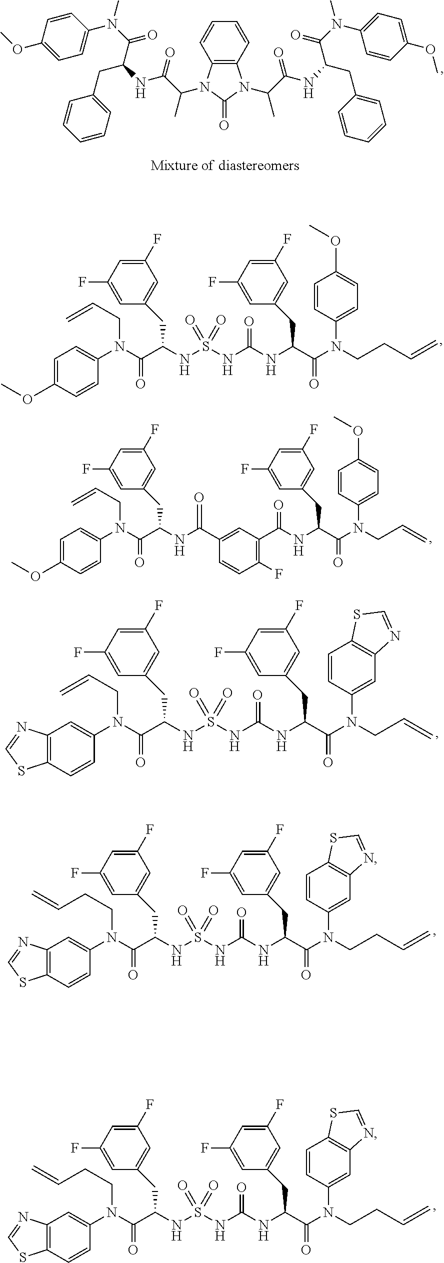

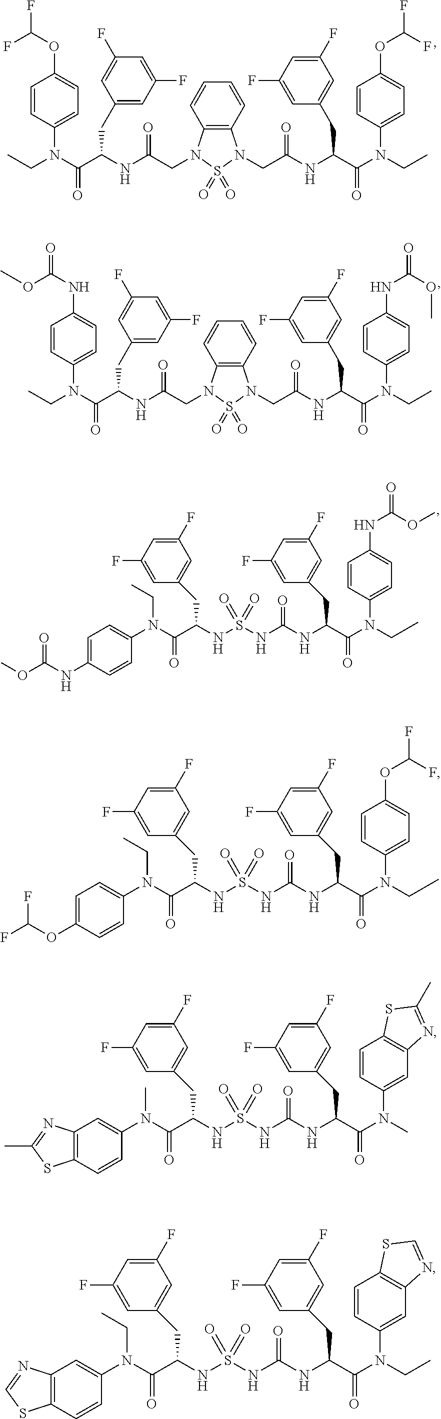

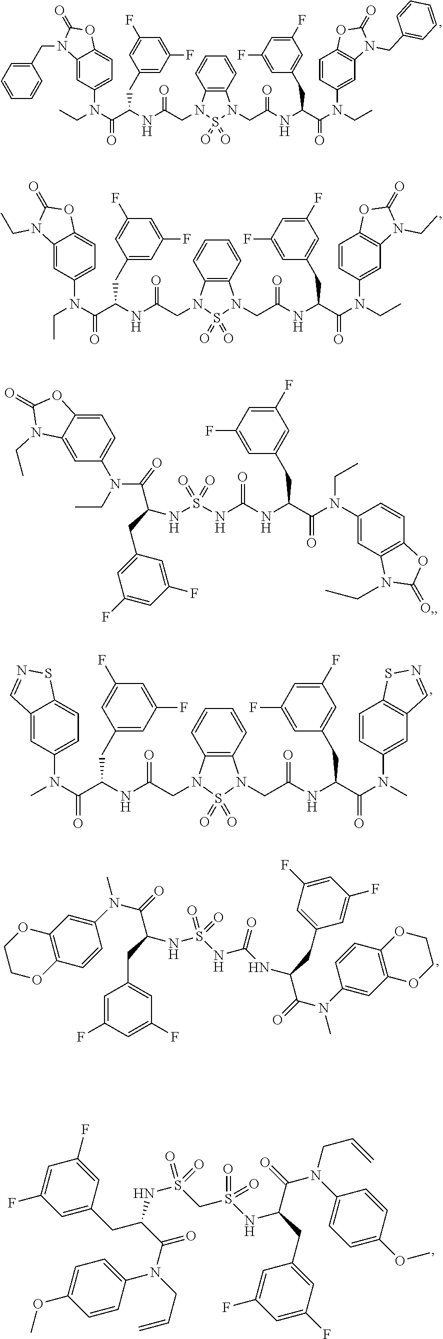

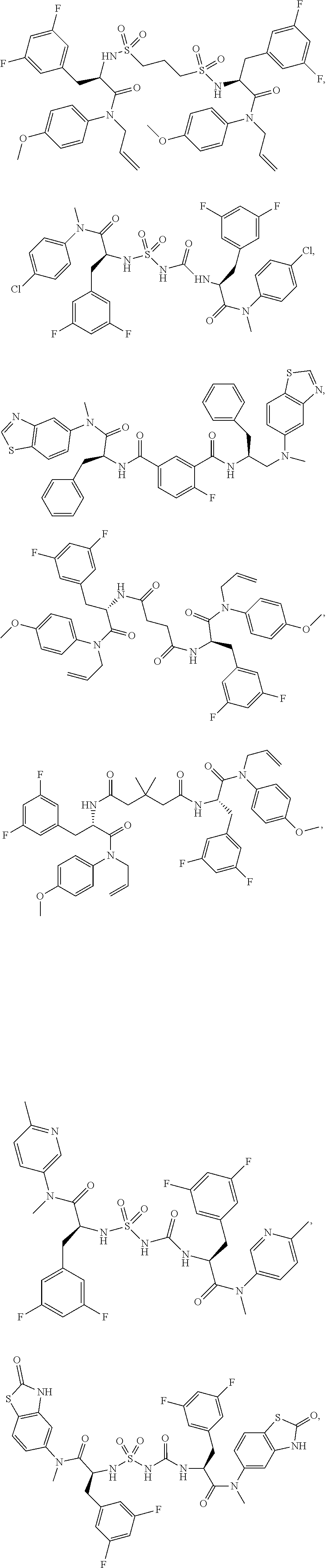

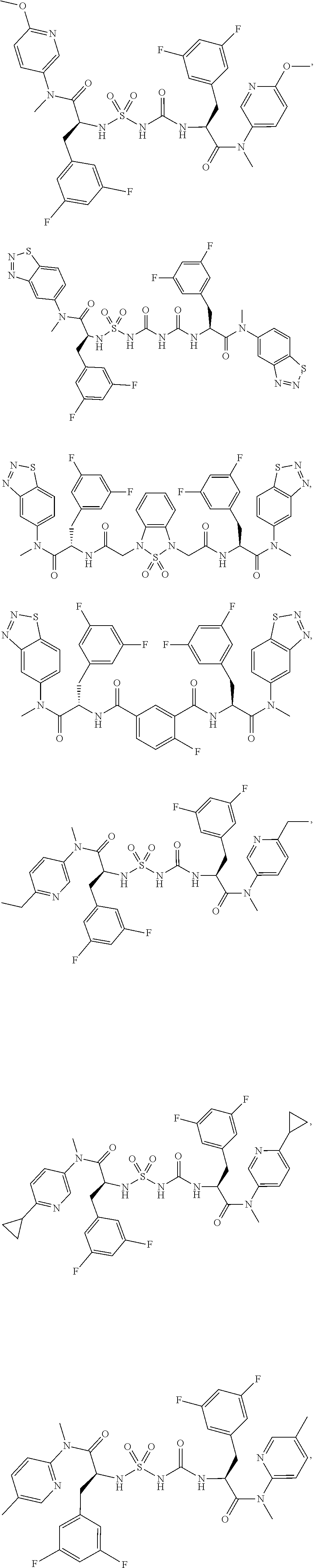

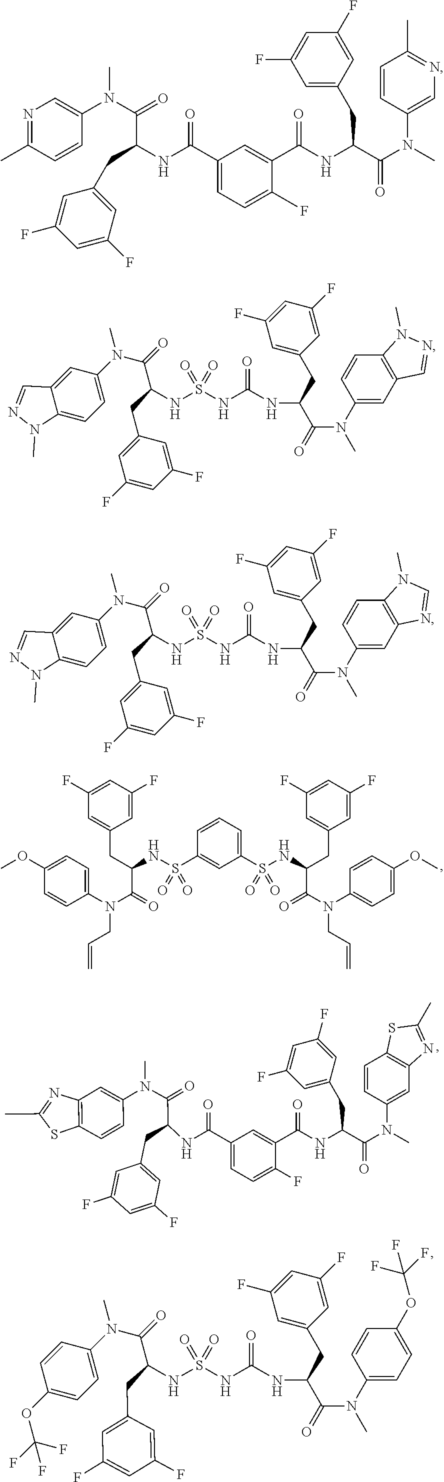

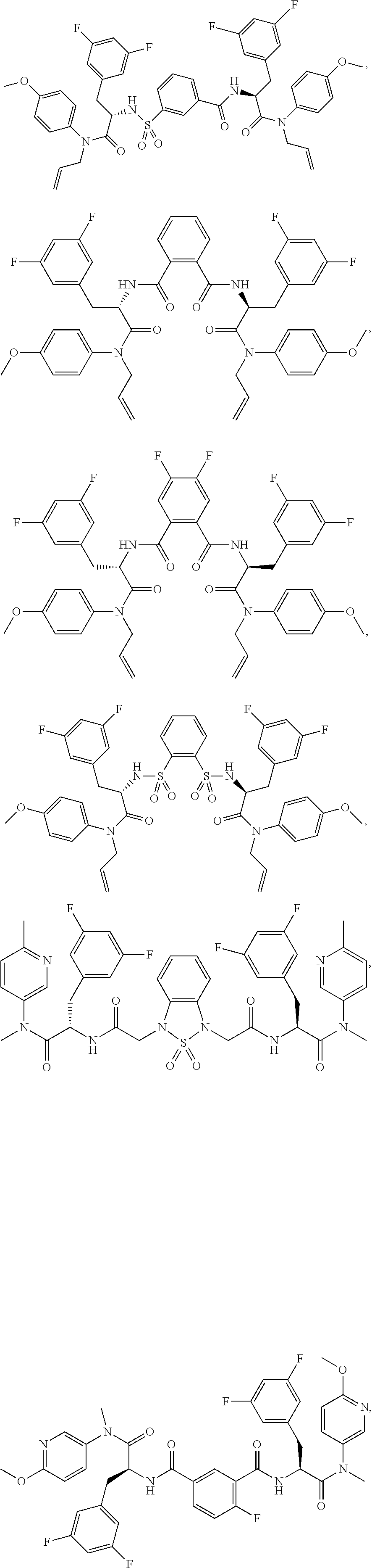

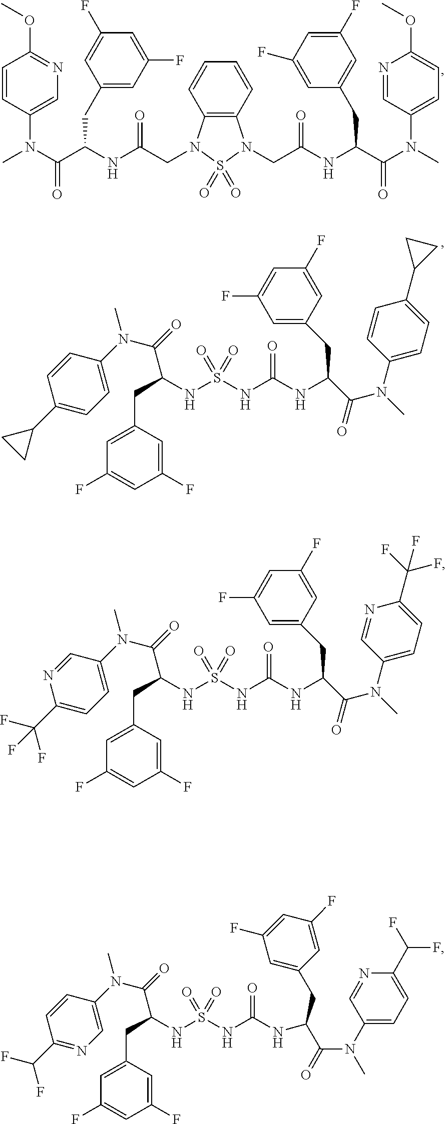 [SMILES: COc1ccc(N(C)C(=O)[C@H](Cc2cc(F)cc(F)c2)NC(=O)CN2c3ccccc3N(CC(=O)N[C@@H](Cc3cc(F)cc(F)c3)C(=O)N(C)c3ccc(OC)nc3)S2(=O)=O)cn1 ; CN(C(=O)[C@H](Cc1cc(F)cc(F)c1)NC(=O)NS(=O)(=O)N[C@@H](Cc1cc(F)cc(F)c1)C(=O)N(C)c1ccc(C2CC2)cc1)c1ccc(C2CC2)cc1 ; CN(C(=O)[C@H](Cc1cc(F)cc(F)c1)NC(=O)NS(=O)(=O)N[C@@H](Cc1cc(F)cc(F)c1)C(=O)N(C)c1ccc(C(F)(F)F)nc1)c1ccc(C(F)(F)F)nc1 ; CN(C(=O)[C@H](Cc1cc(F)cc(F)c1)NC(=O)NS(=O)(=O)N[C@@H](Cc1cc(F)cc(F)c1)C(=O)N(C)c1ccc(C(F)F)nc1)c1ccc(C(F)F)nc1]

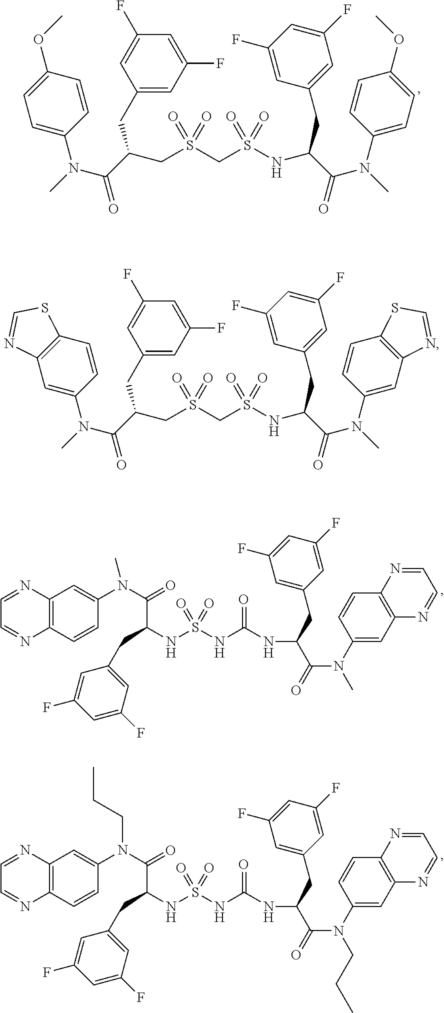 [SMILES: COc1ccc(N(C)C(=O)[C@H](Cc2cc(F)cc(F)c2)CS(=O)(=O)CS(=O)(=O)N[C@@H](Cc2cc(F)cc(F)c2)C(=O)N(C)c2ccc(OC)cc2)cc1 ; CN(C(=O)[C@H](Cc1cc(F)cc(F)c1)CS(=O)(=O)CS(=O)(=O)N[C@@H](Cc1cc(F)cc(F)c1)C(=O)N(C)c1ccc2scnc2c1)c1ccc2scnc2c1 ; CN(C(=O)[C@H](Cc1cc(F)cc(F)c1)NC(=O)NS(=O)(=O)N[C@@H](Cc1cc(F)cc(F)c1)C(=O)N(C)c1ccc2nccnc2c1)c1ccc2nccnc2c1 ; CCCN(C(=O)[C@H](Cc1cc(F)cc(F)c1)NC(=O)NS(=O)(=O)N[C@@H](Cc1cc(F)cc(F)c1)C(=O)N(CCC)c1ccc2nccnc2c1)c1ccc2nccnc2c1]

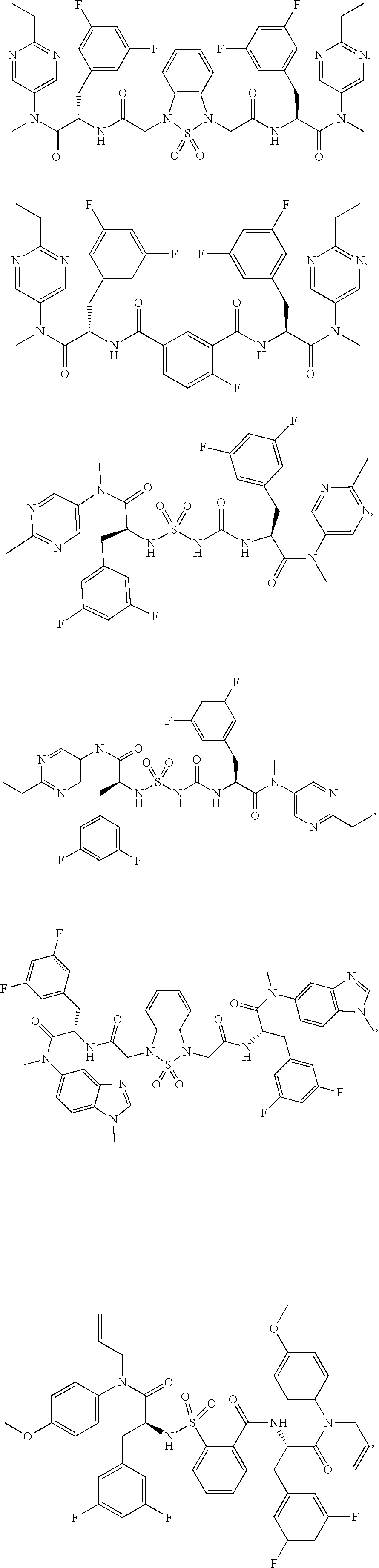

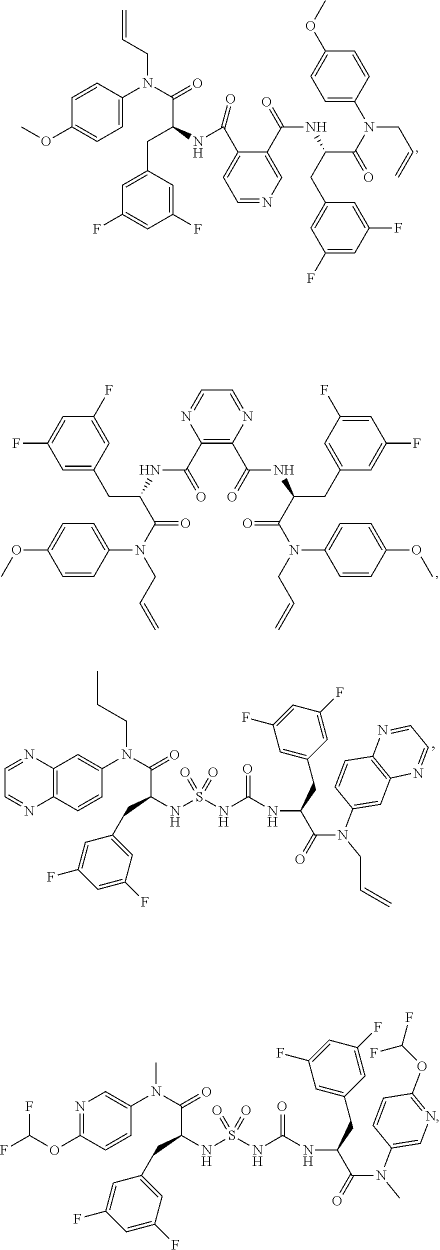

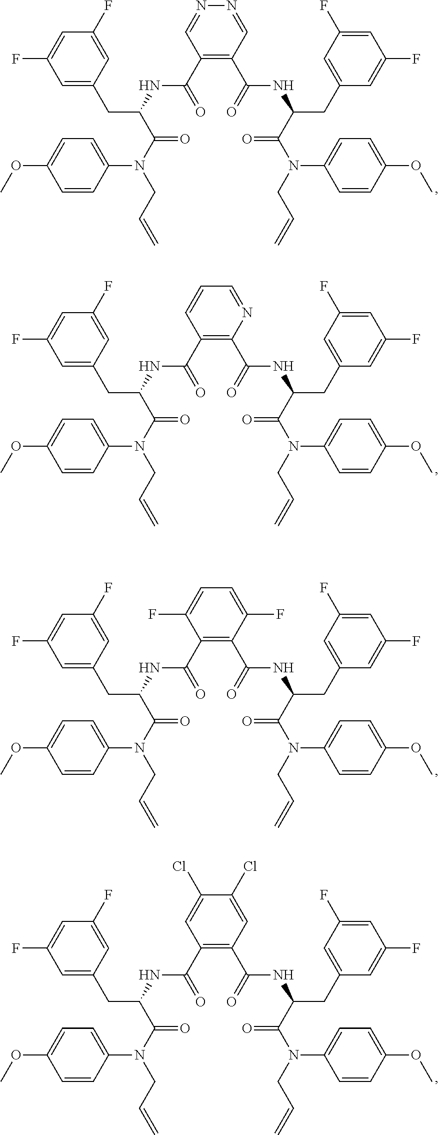

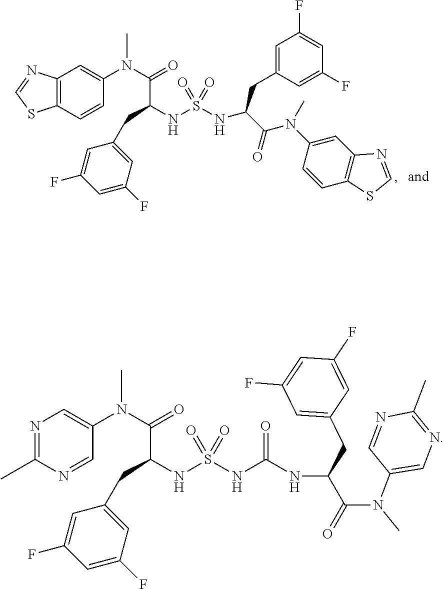 [SMILES: CN(C(=O)[C@H](Cc1cc(F)cc(F)c1)NS(=O)(=O)N[C@@H](Cc1cc(F)cc(F)c1)C(=O)N(C)c1ccc2scnc2c1)c1ccc2scnc2c1 ; Cc1ncc(N(C)C(=O)[C@H](Cc2cc(F)cc(F)c2)NC(=O)NS(=O)(=O)N[C@@H](Cc2cc(F)cc(F)c2)C(=O)N(C)c2cnc(C)nc2)cn1]

Other preferred compounds, including pharmaceutically acceptable salts thereof, are selected from the group of:

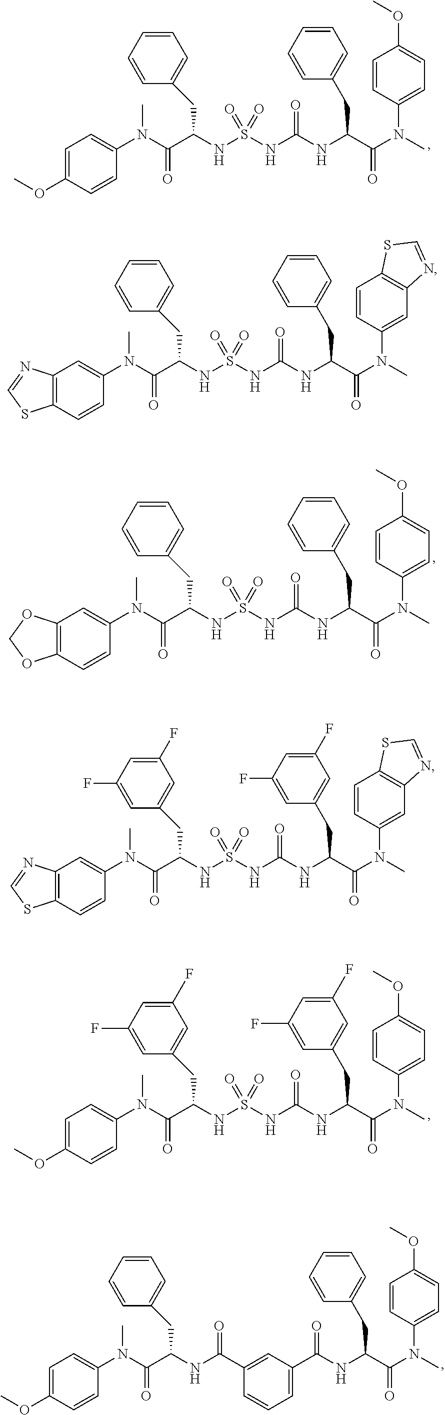

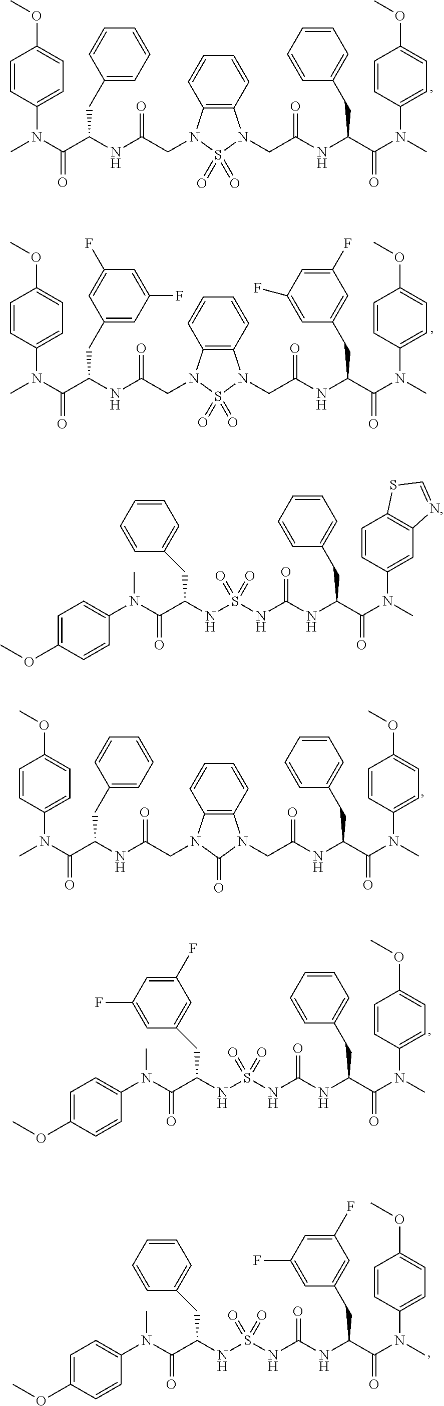

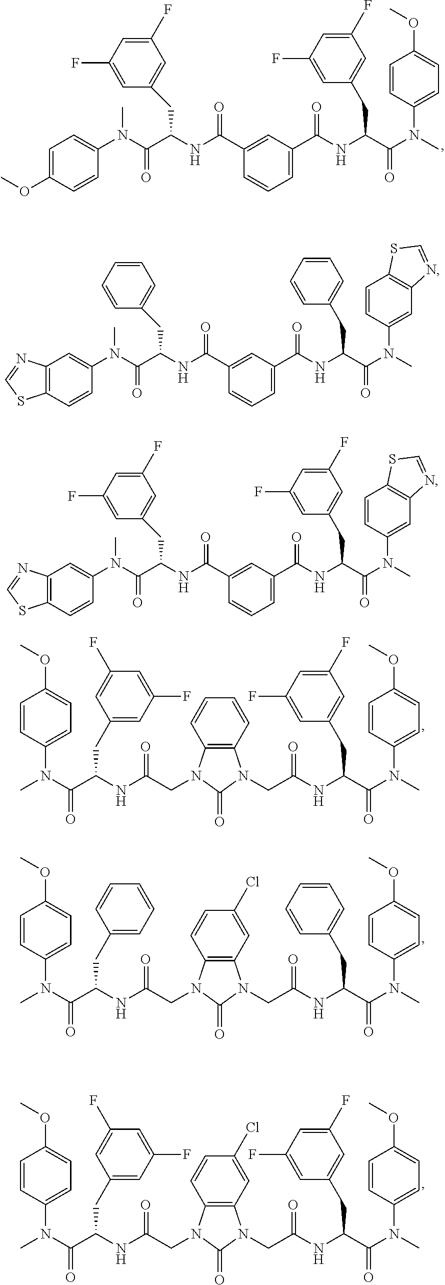

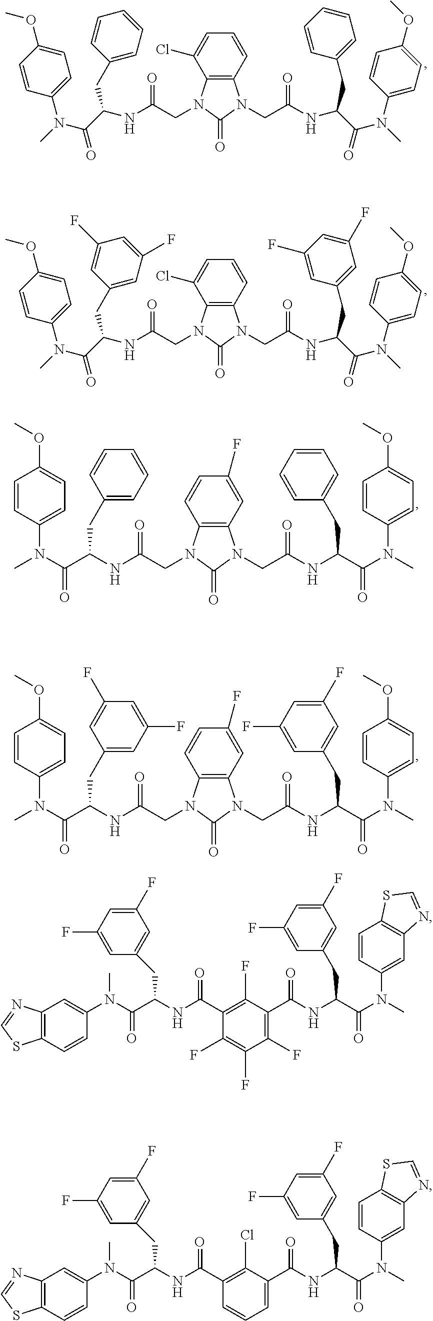

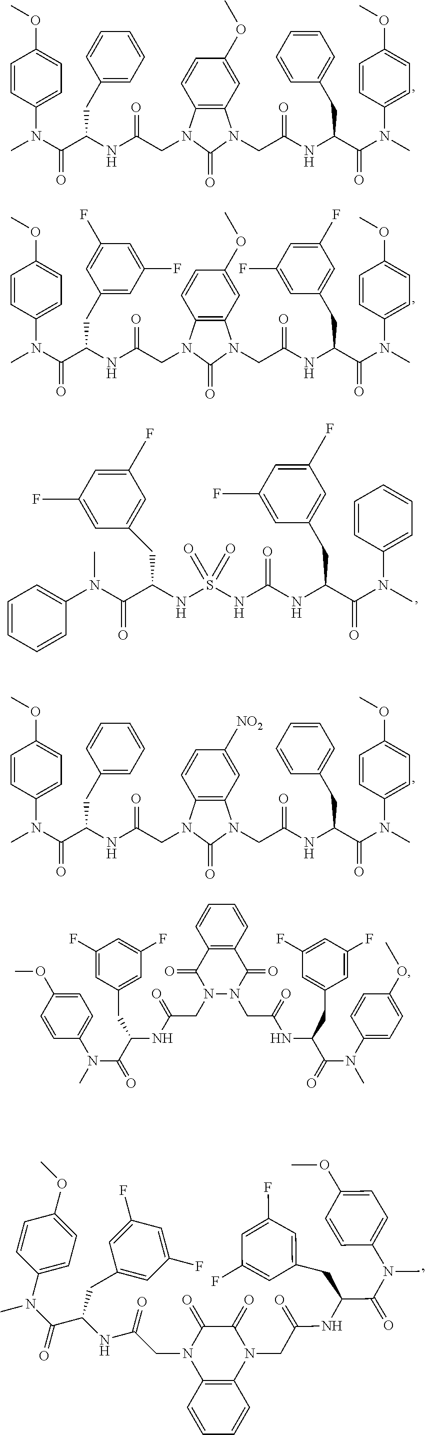

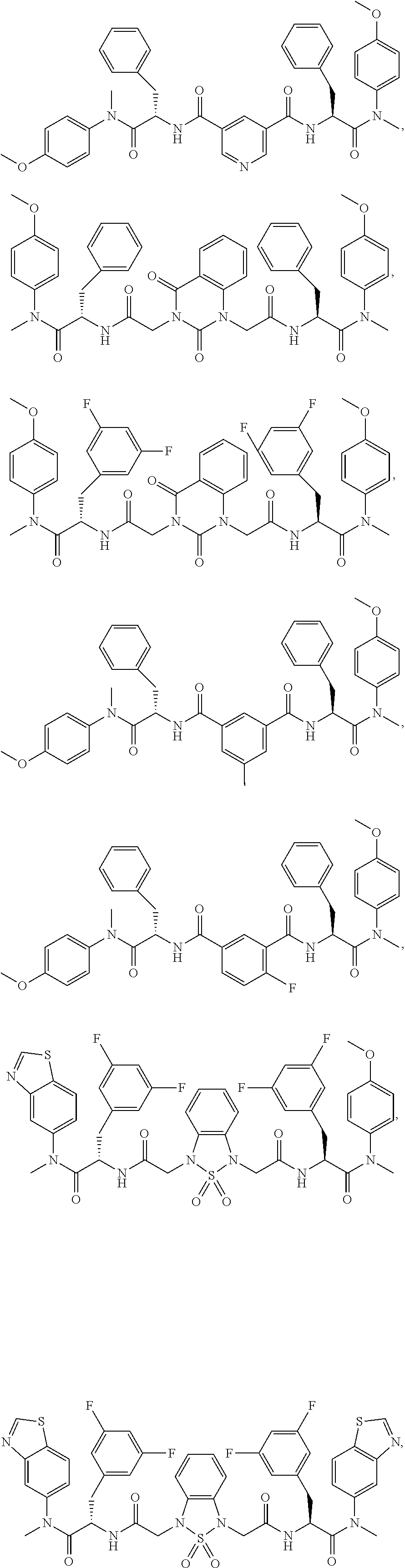

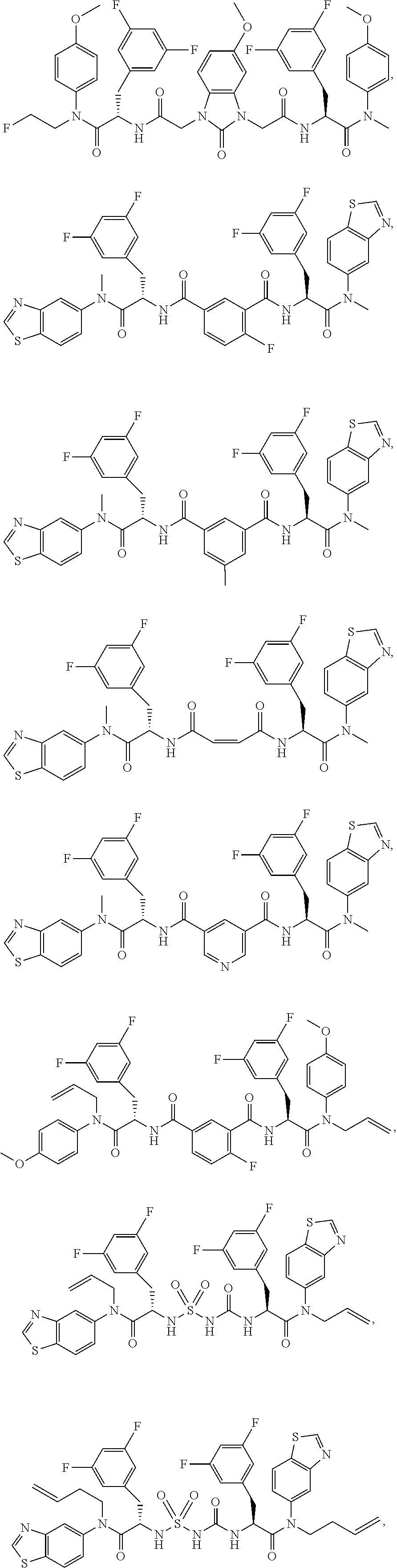

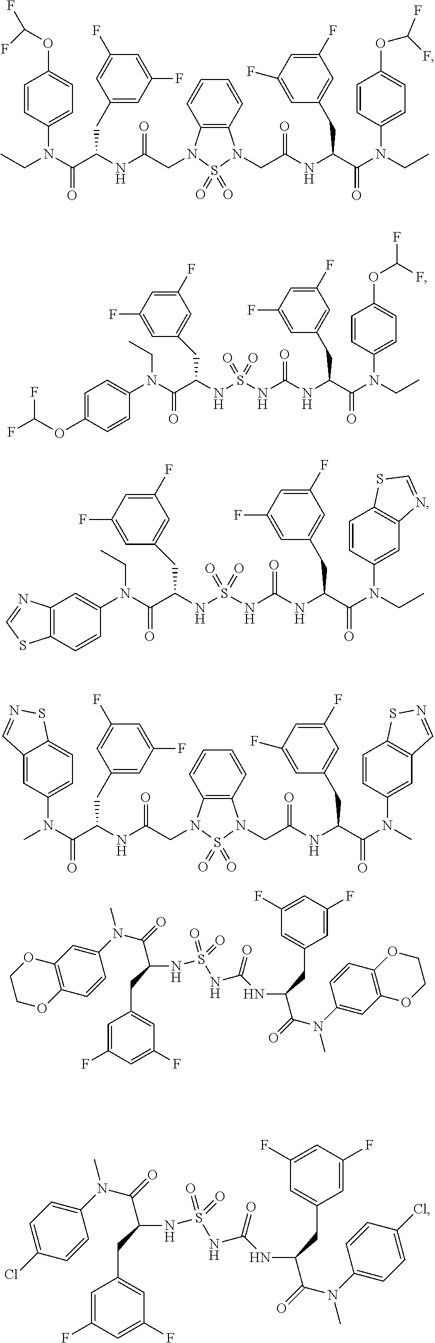

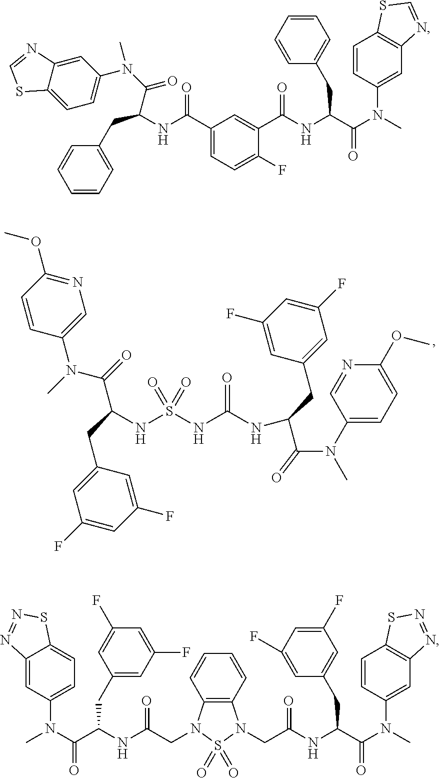

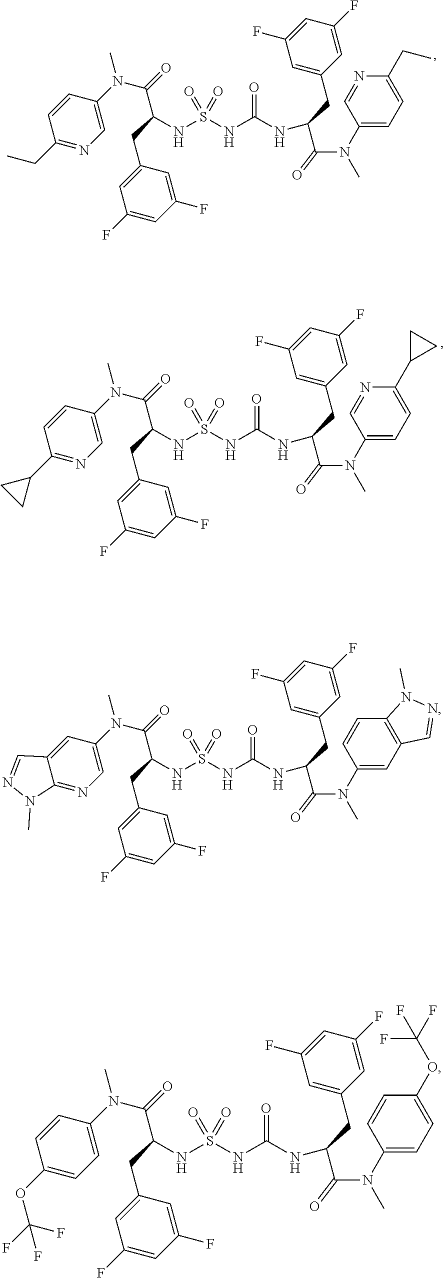 [SMILES: CCc1ccc(N(C)C(=O)[C@H](Cc2cc(F)cc(F)c2)NC(=O)NS(=O)(=O)N[C@@H](Cc2cc(F)cc(F)c2)C(=O)N(C)c2ccc(CC)nc2)cn1 ; CN(C(=O)[C@H](Cc1cc(F)cc(F)c1)NC(=O)NS(=O)(=O)N[C@@H](Cc1cc(F)cc(F)c1)C(=O)N(C)c1ccc(C2CC2)nc1)c1ccc(C2CC2)nc1 ; CN(C(=O)[C@H](Cc1cc(F)cc(F)c1)NC(=O)NS(=O)(=O)N[C@@H](Cc1cc(F)cc(F)c1)C(=O)N(C)c1cnc2c(cnn2C)c1)c1ccc2c(cnn2C)c1 ; CN(C(=O)[C@H](Cc1cc(F)cc(F)c1)NC(=O)NS(=O)(=O)N[C@@H](Cc1cc(F)cc(F)c1)C(=O)N(C)c1ccc(OC(F)(F)F)cc1)c1ccc(OC(F)(F)F)cc1]

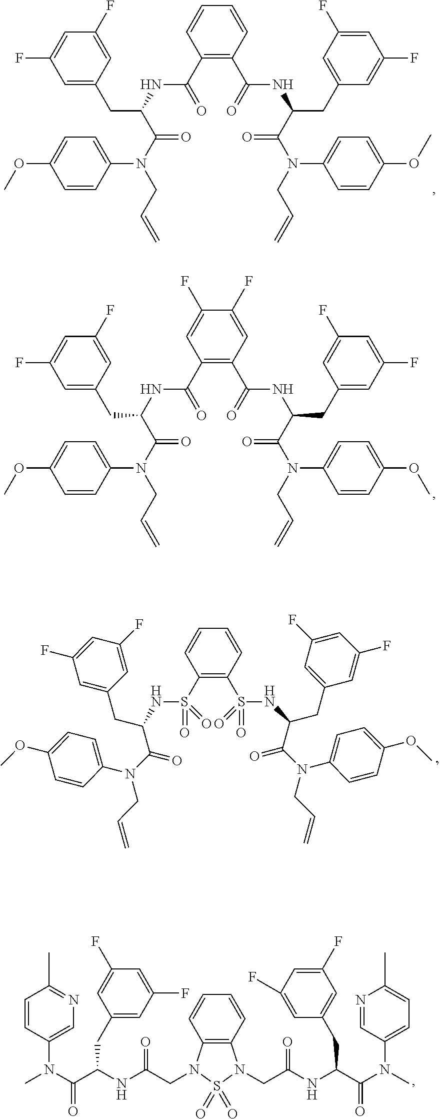

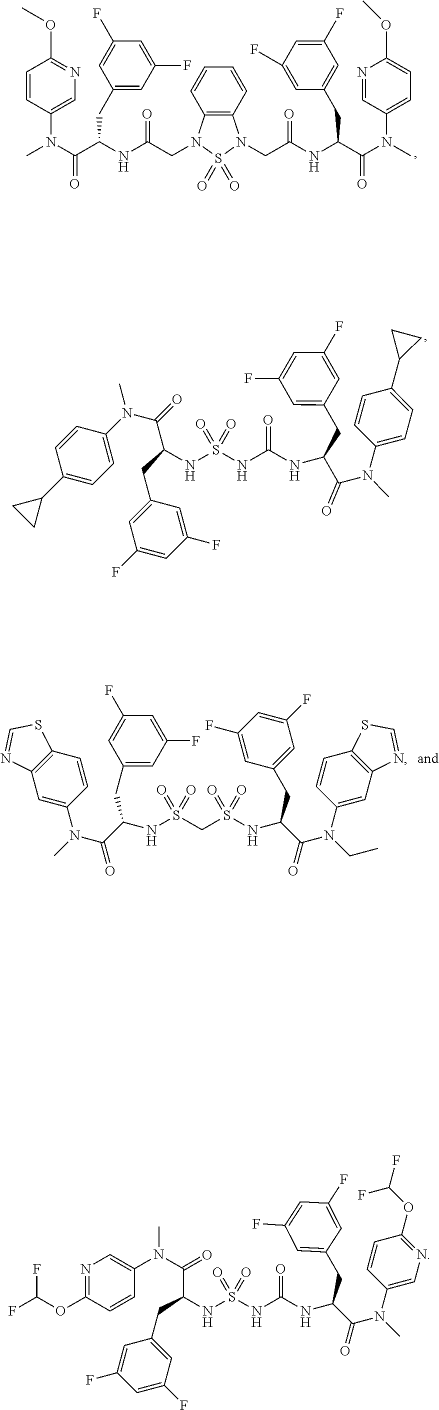 [SMILES: COc1ccc(N(C)C(=O)[C@H](Cc2cc(F)cc(F)c2)NC(=O)CN2c3ccccc3N(CC(=O)N[C@@H](Cc3cc(F)cc(F)c3)C(=O)N(C)c3ccc(OC)nc3)S2(=O)=O)cn1 ; CN(C(=O)[C@H](Cc1cc(F)cc(F)c1)NC(=O)NS(=O)(=O)N[C@@H](Cc1cc(F)cc(F)c1)C(=O)N(C)c1ccc(C2CC2)cc1)c1ccc(C2CC2)cc1 ; CCN(C(=O)[C@H](Cc1cc(F)cc(F)c1)NS(=O)(=O)CS(=O)(=O)N[C@@H](Cc1cc(F)cc(F)c1)C(=O)N(C)c1ccc2scnc2c1)c1ccc2scnc2c1 ; "and" ; CN(C(=O)[C@H](Cc1cc(F)cc(F)c1)NC(=O)NS(=O)(=O)N[C@@H](Cc1cc(F)cc(F)c1)C(=O)N(C)c1ccc(OC(F)F)nc1)c1ccc(OC(F)F)nc1]

A compound which is selected from the group of

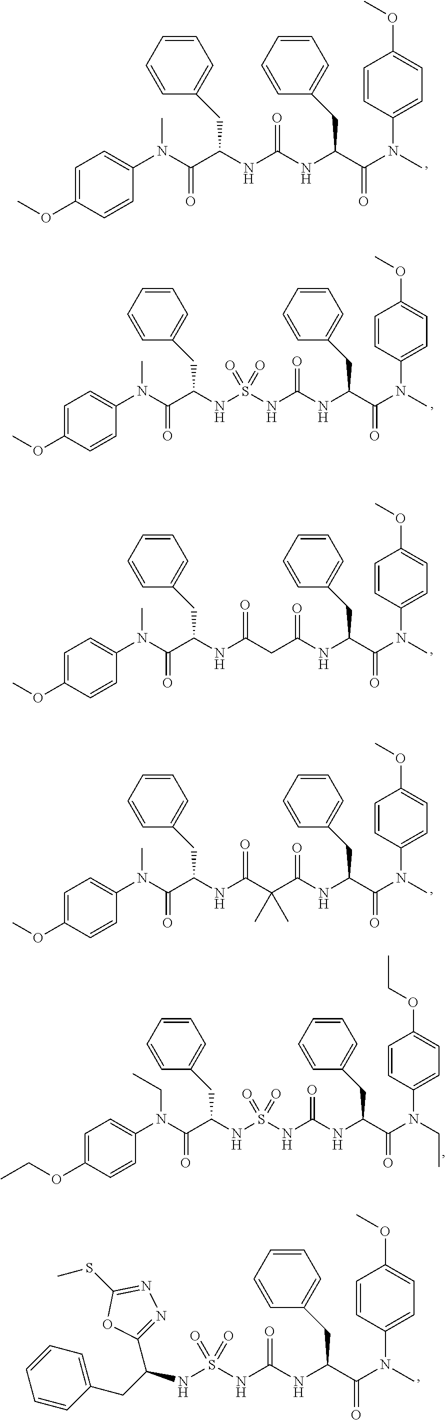

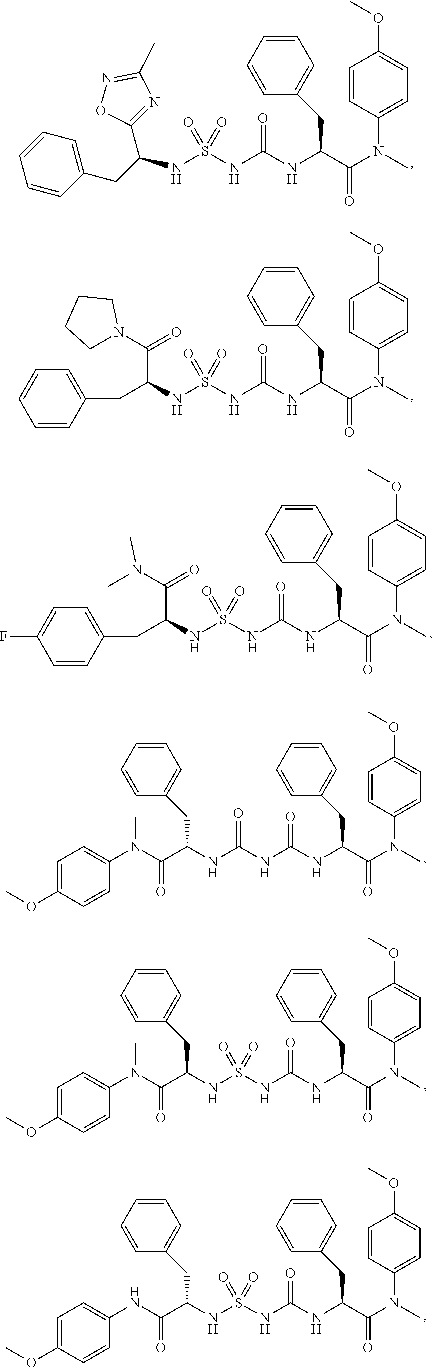

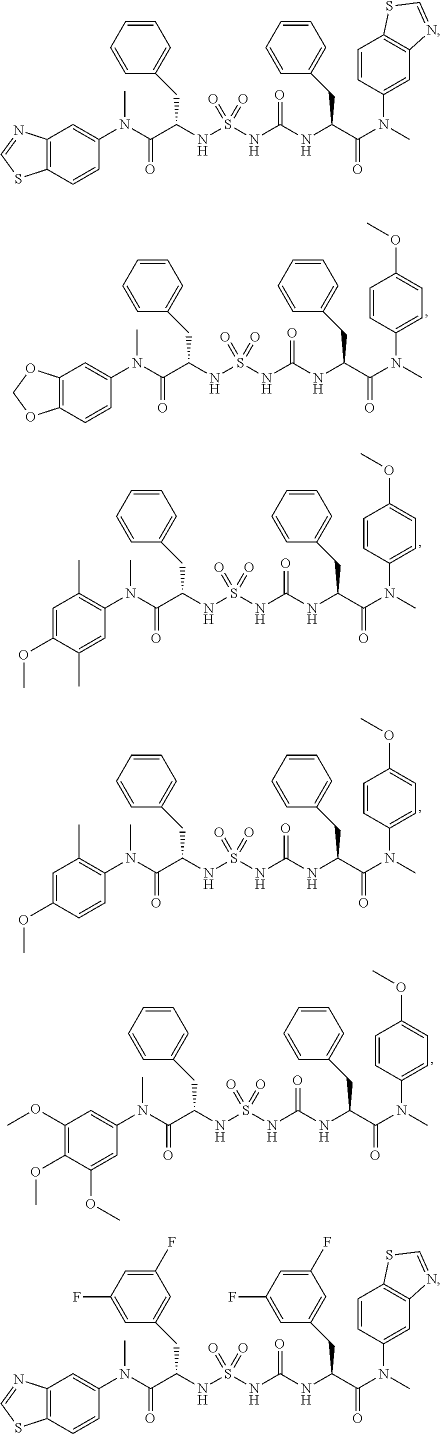

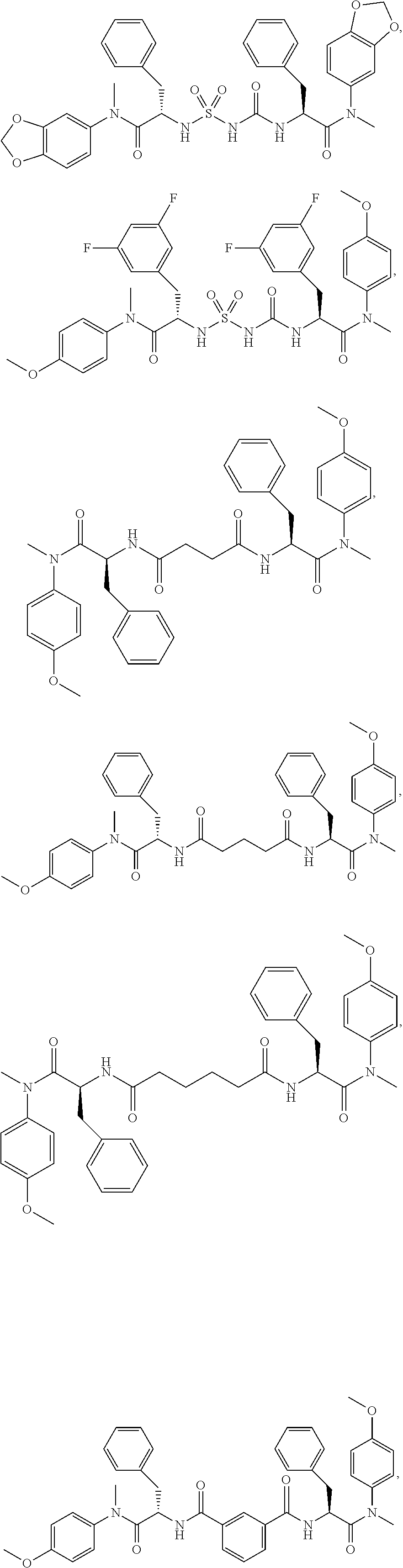

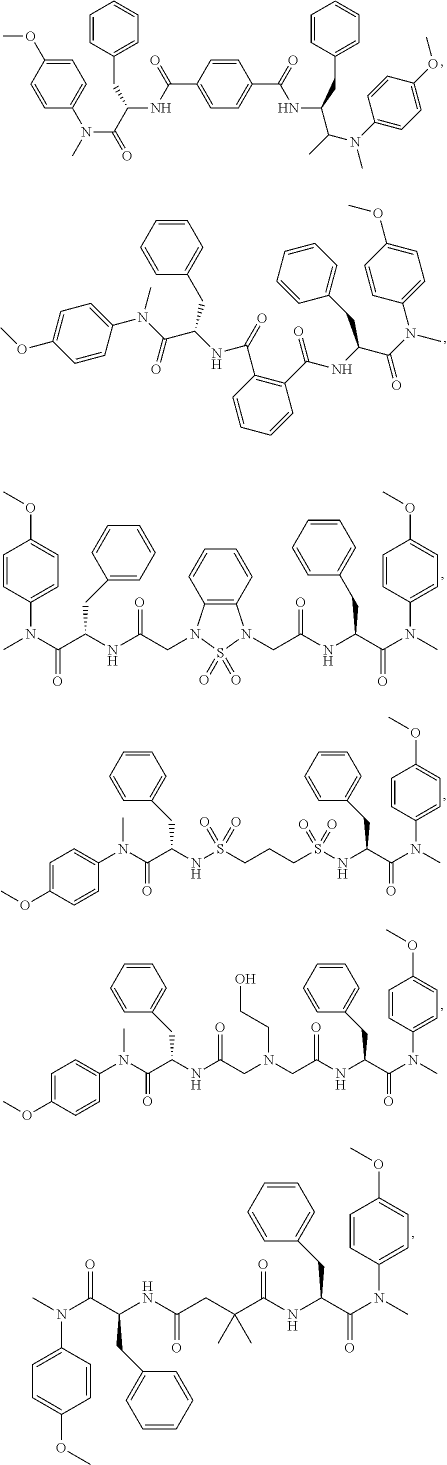

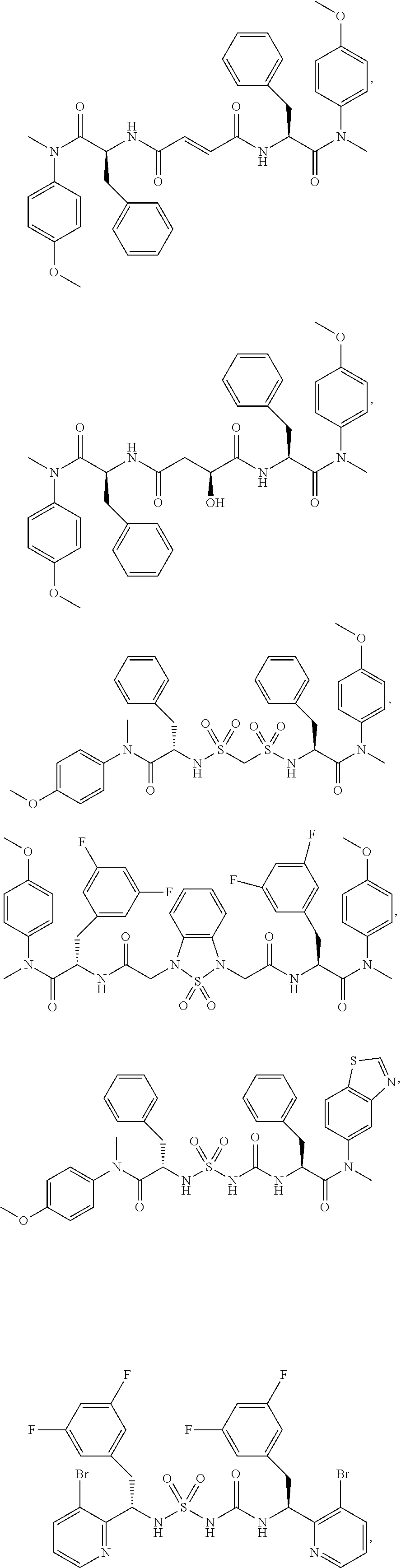

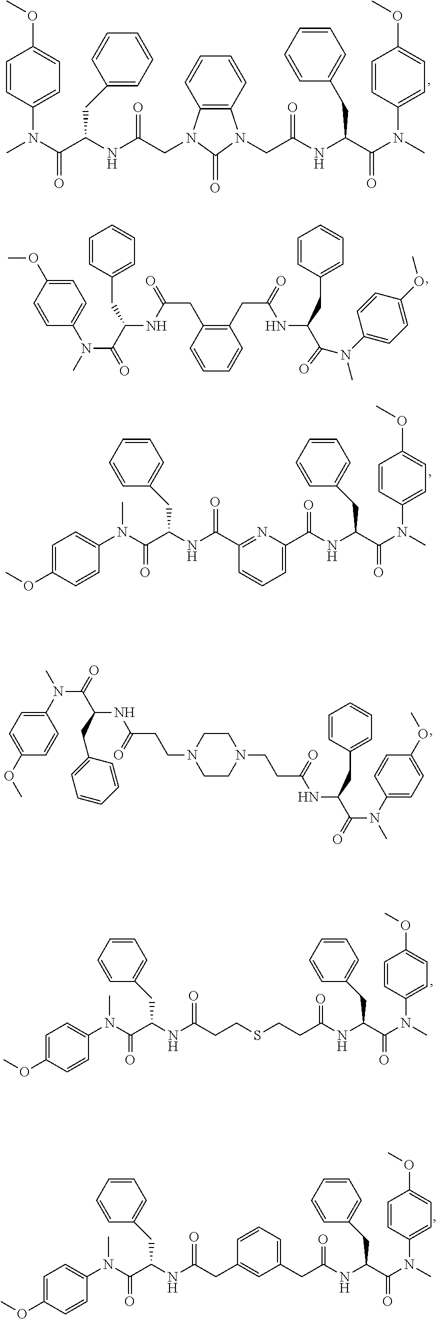

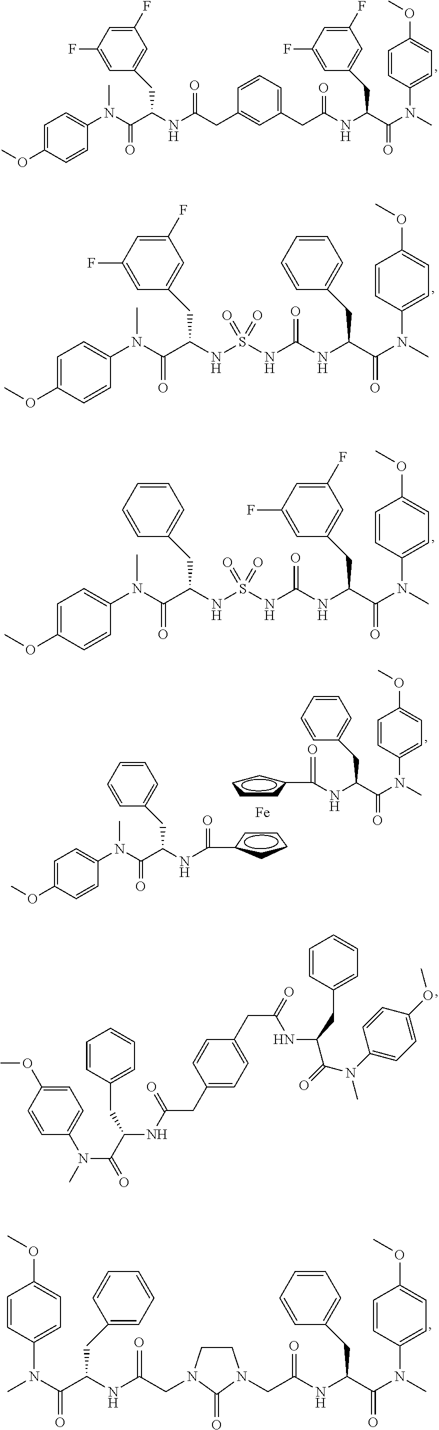

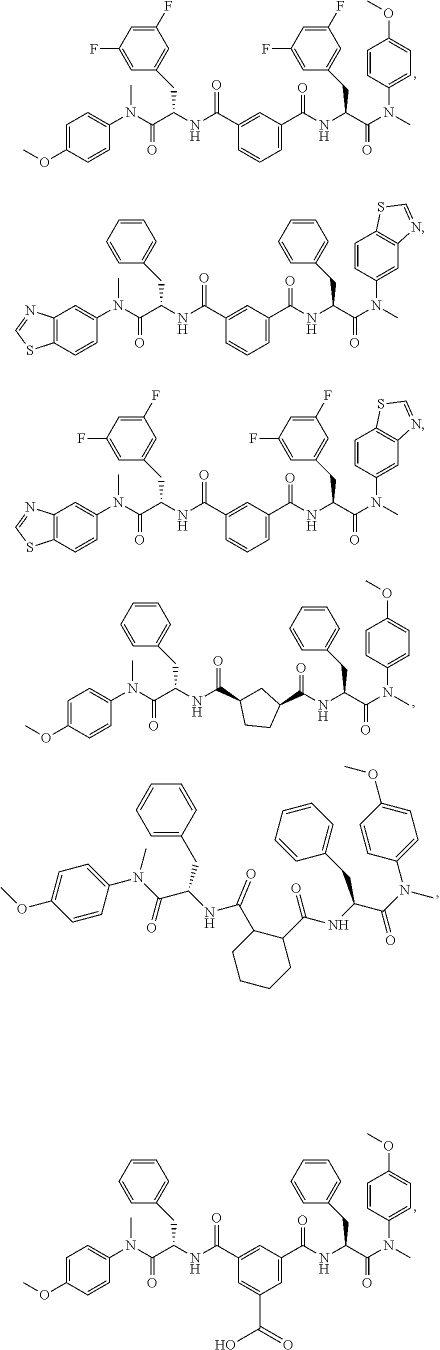

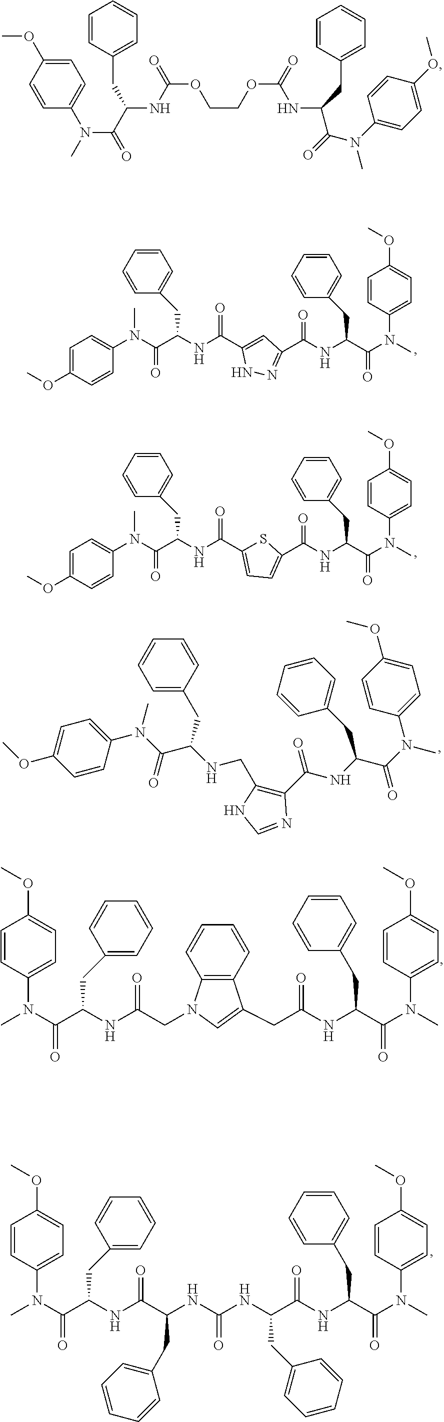

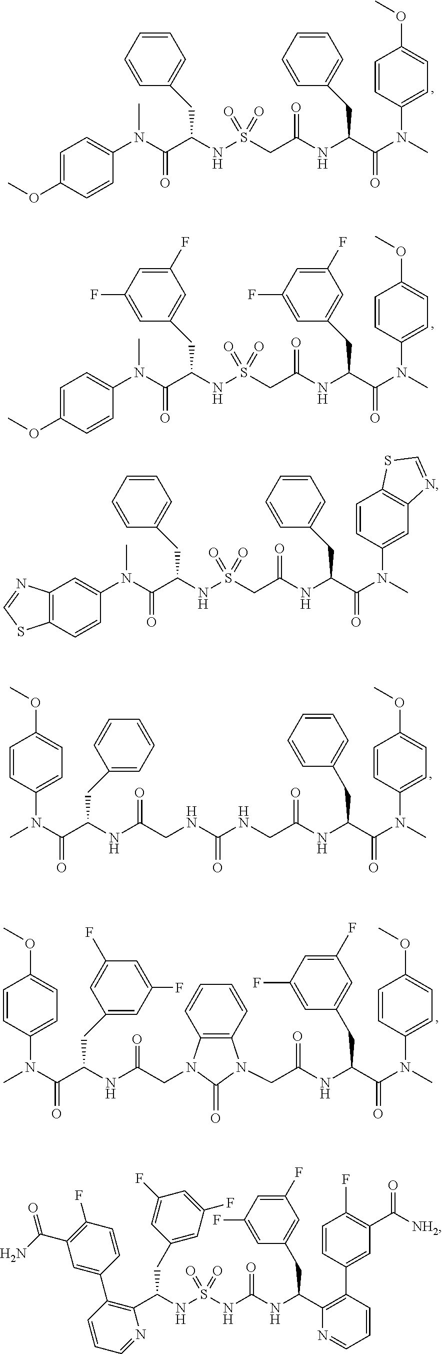

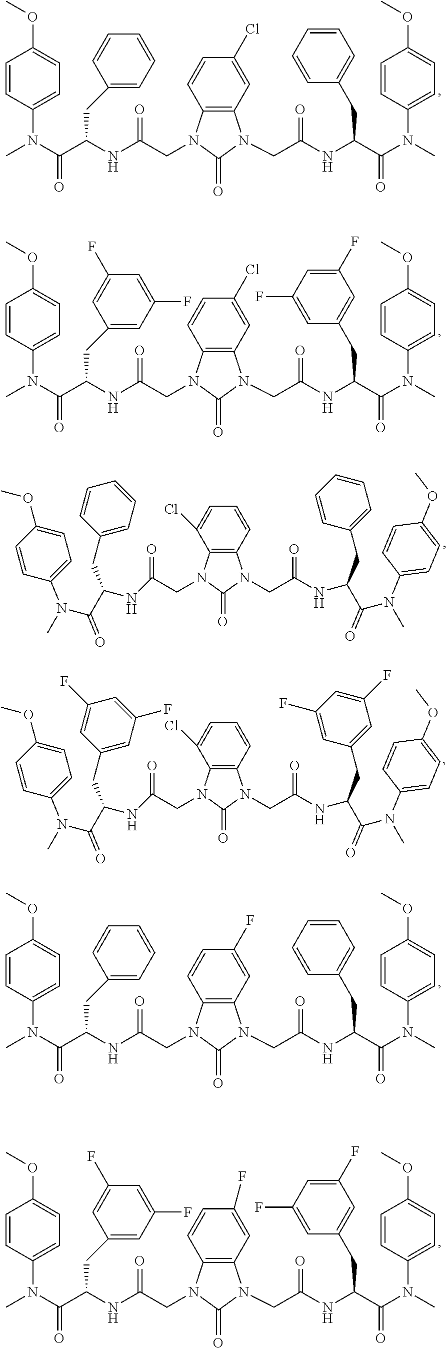

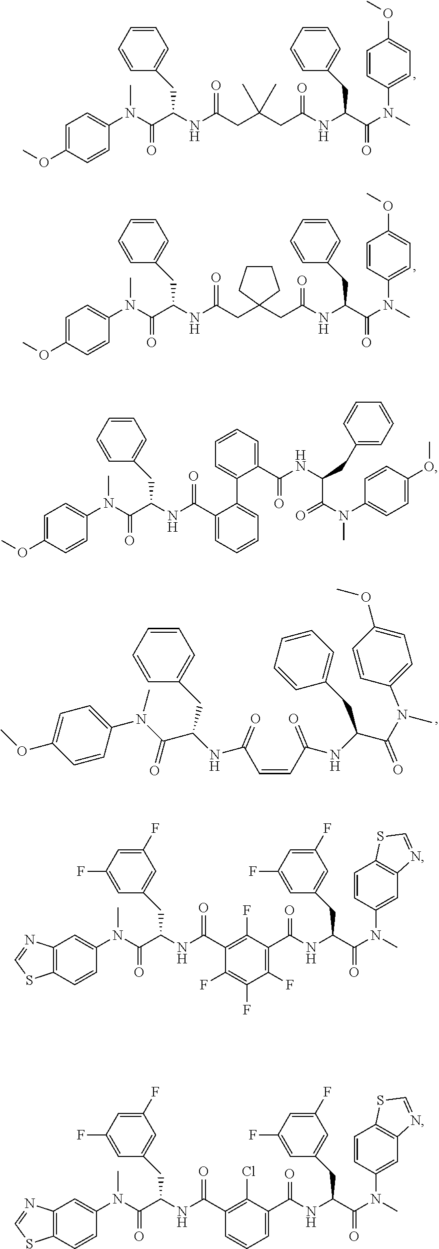

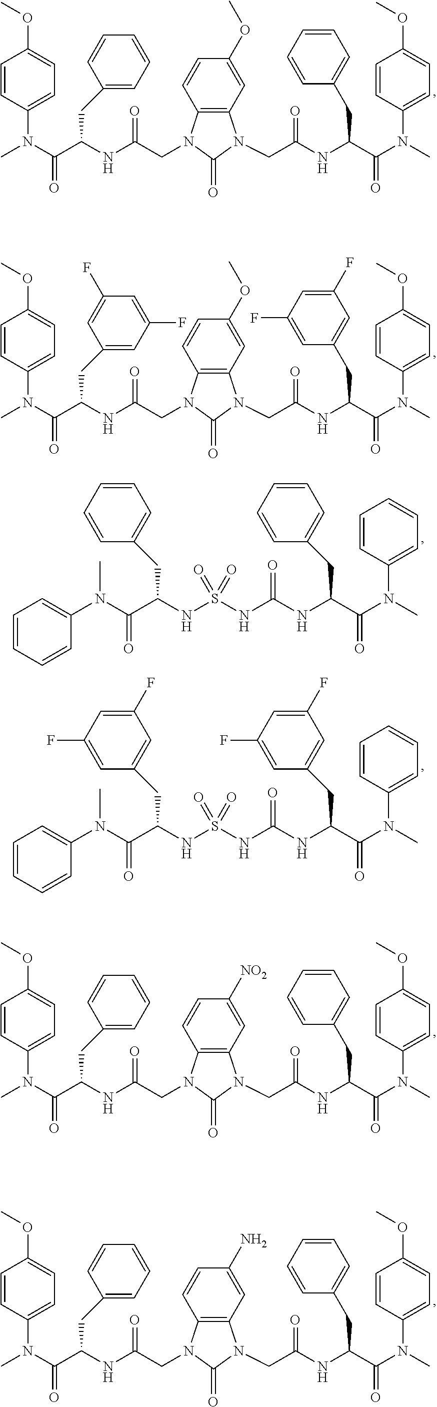

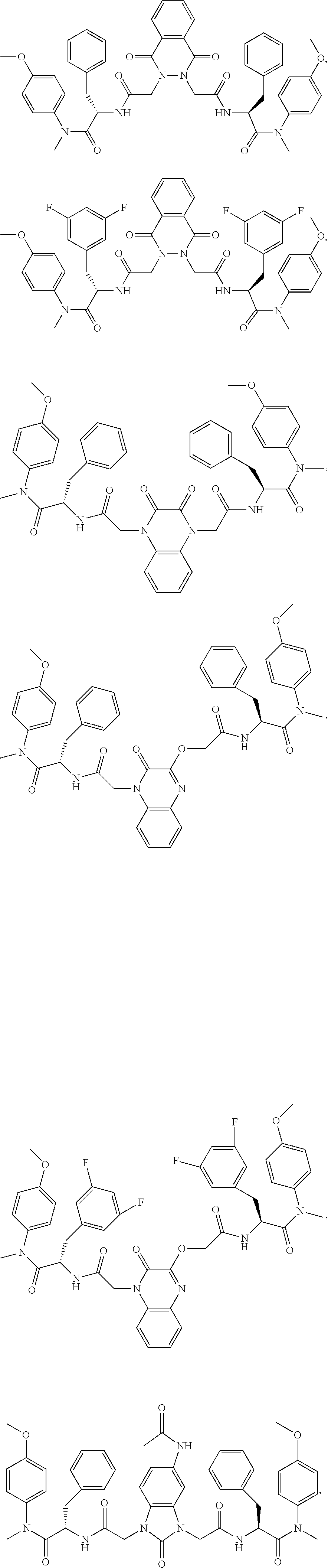

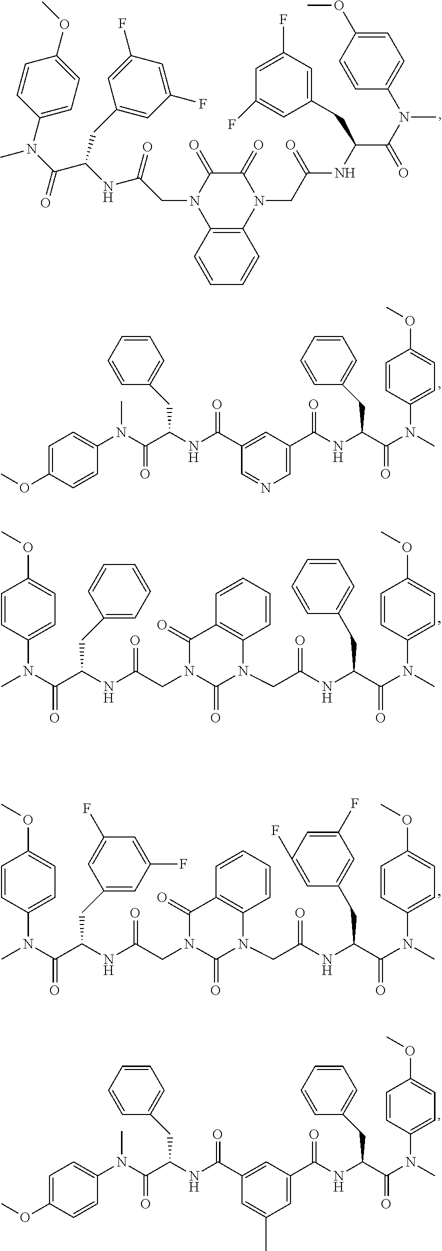

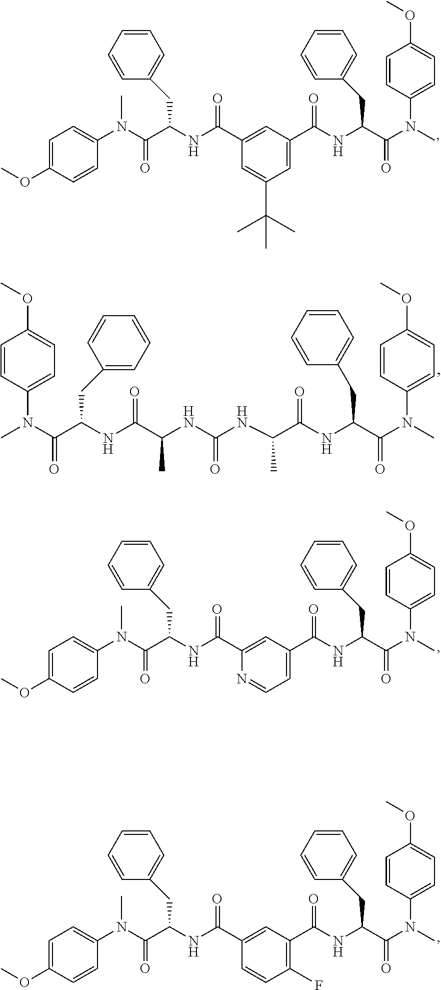 [SMILES: COc1ccc(N(C)C(=O)[C@H](Cc2ccccc2)NC(=O)c2cc(C(=O)N[C@@H](Cc3ccccc3)C(=O)N(C)c3ccc(OC)cc3)cc(C(C)(C)C)c2)cc1 ; COc1ccc(N(C)C(=O)[C@H](Cc2ccccc2)NC(=O)[C@H](C)NC(=O)N[C@@H](C)C(=O)N[C@@H](Cc2ccccc2)C(=O)N(C)c2ccc(OC)cc2)cc1 ; COc1ccc(N(C)C(=O)[C@H](Cc2ccccc2)NC(=O)c2ccnc(C(=O)N[C@@H](Cc3ccccc3)C(=O)N(C)c3ccc(OC)cc3)c2)cc1 ; COc1ccc(N(C)C(=O)[C@H](Cc2ccccc2)NC(=O)c2ccc(F)c(C(=O)N[C@@H](Cc3ccccc3)C(=O)N(C)c3ccc(OC)cc3)c2)cc1]

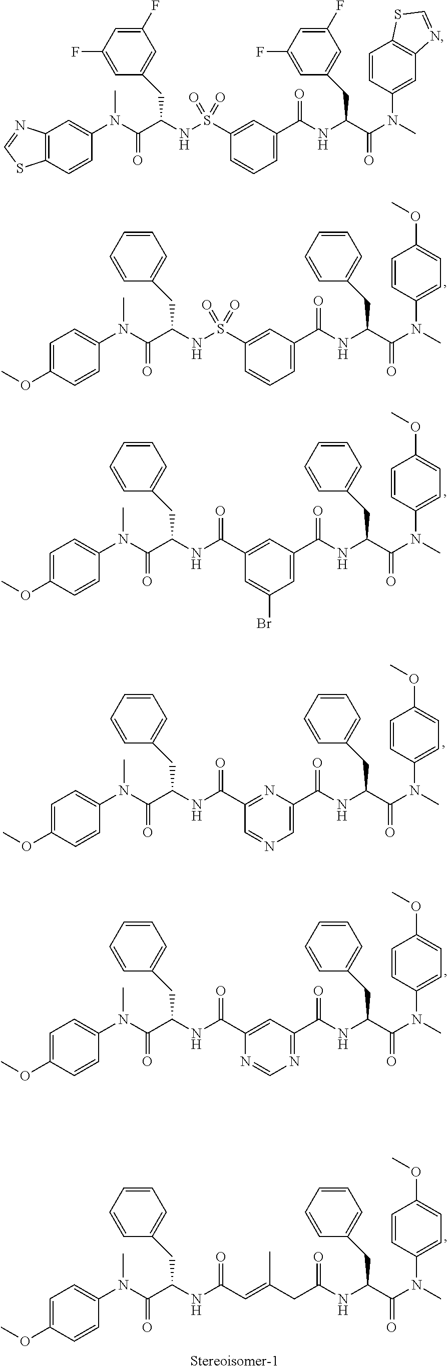

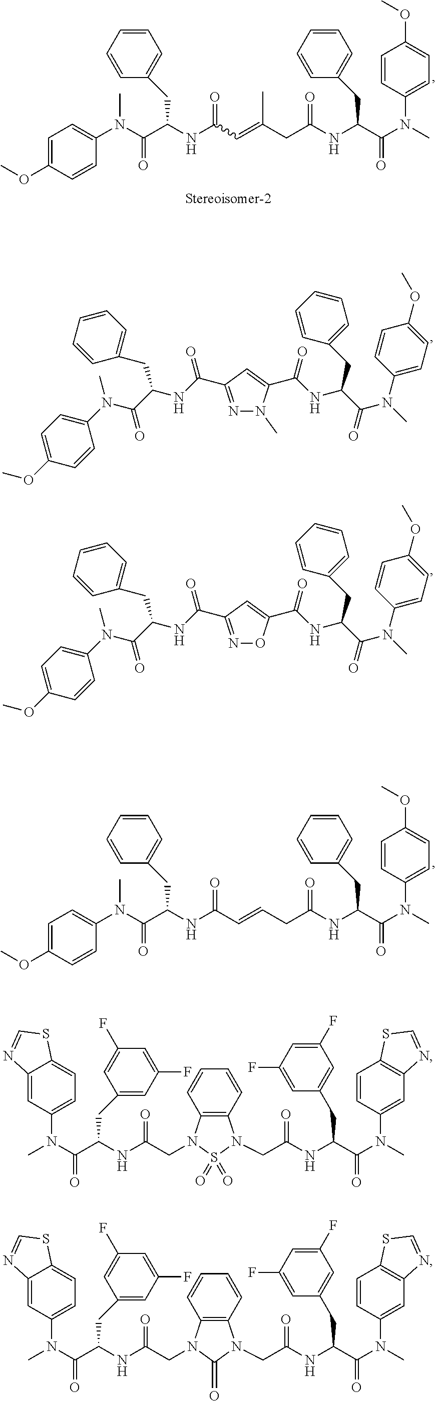

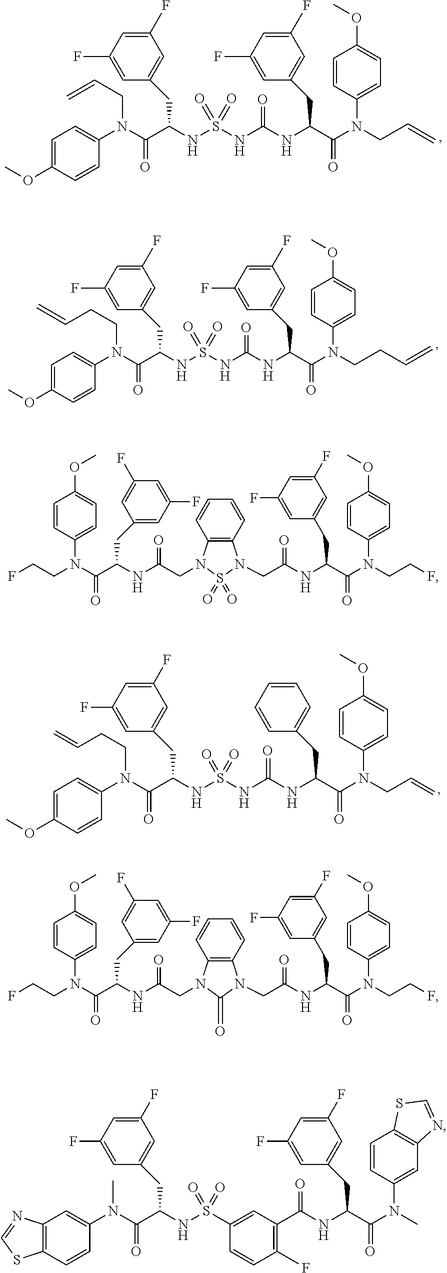

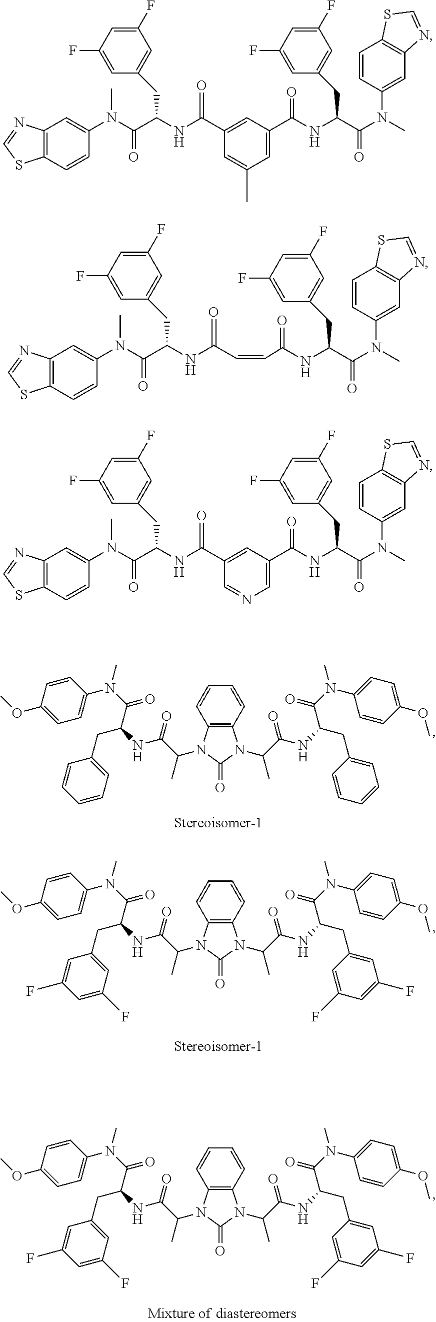

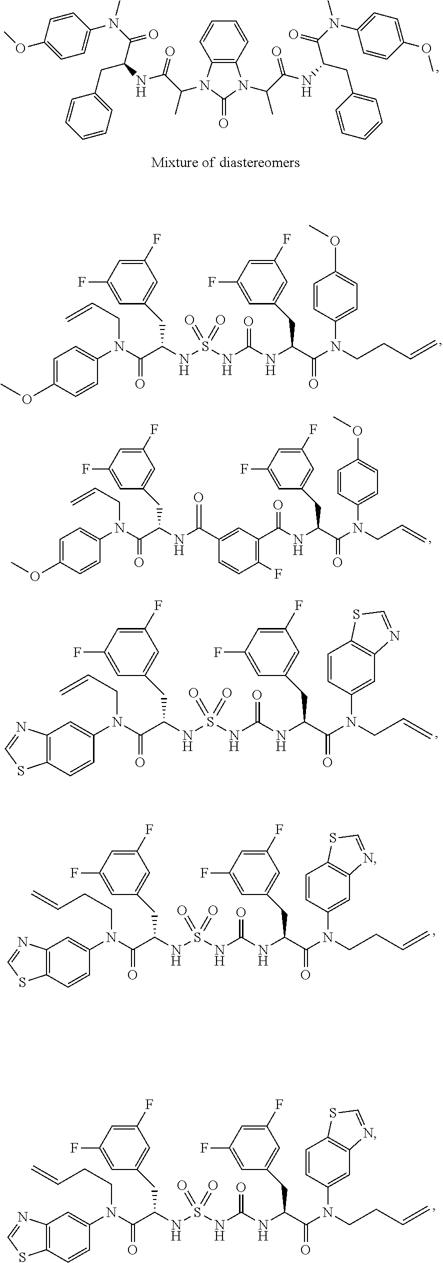

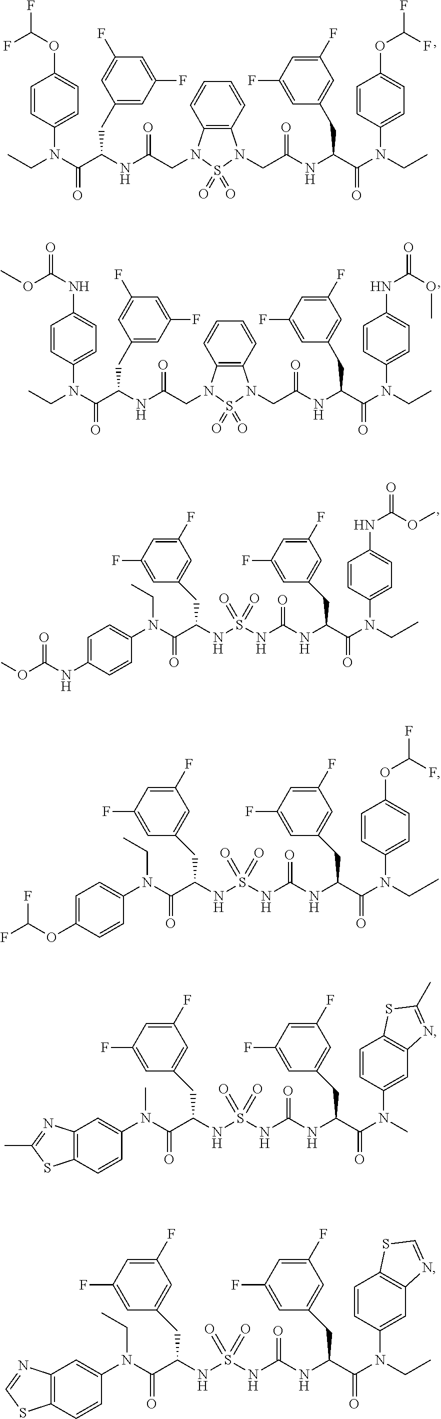

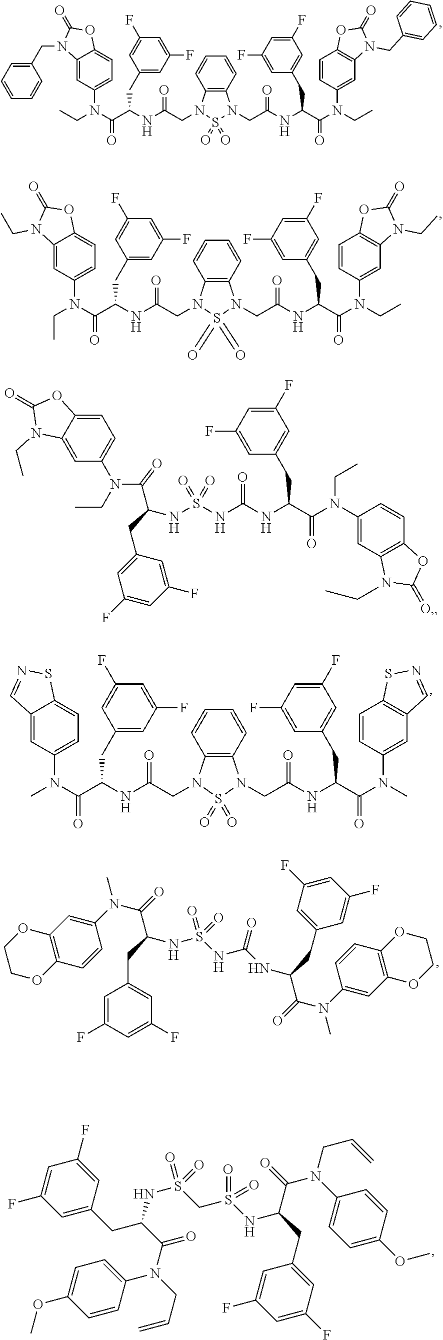

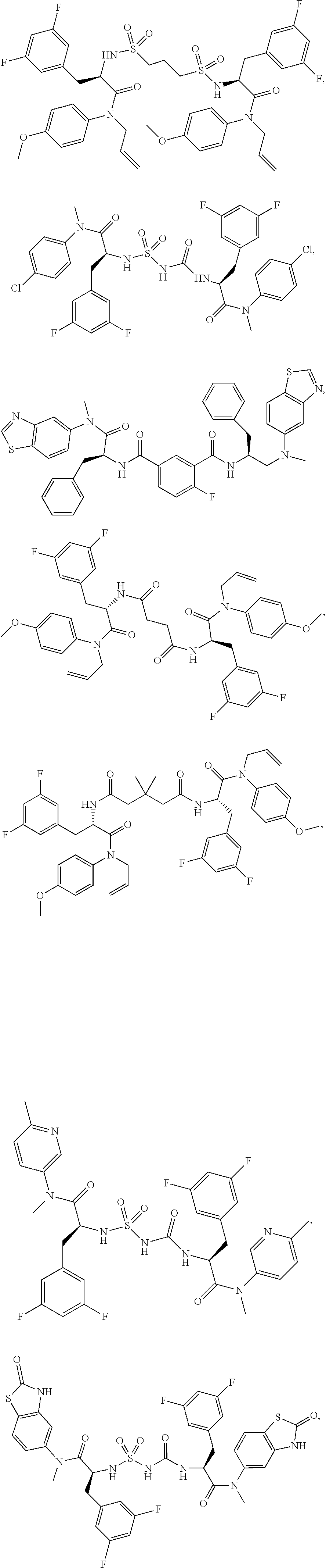

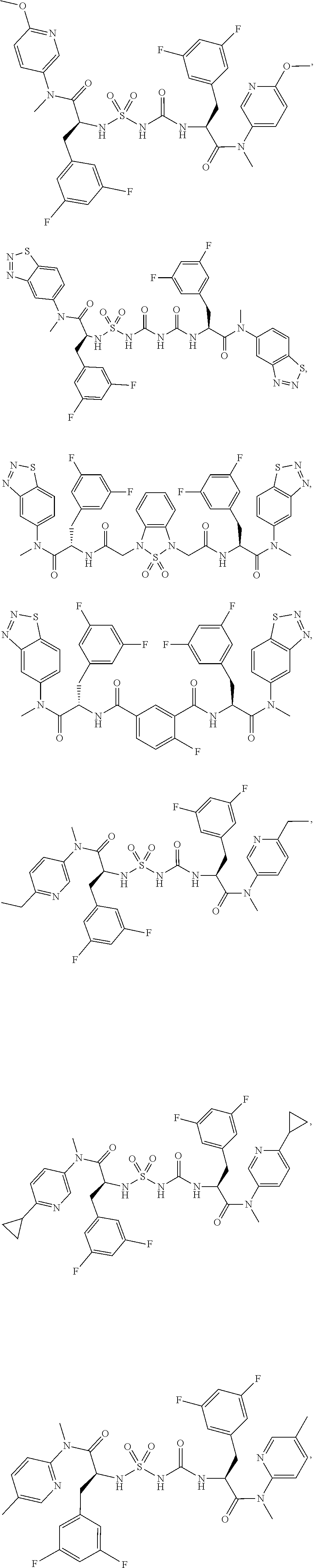

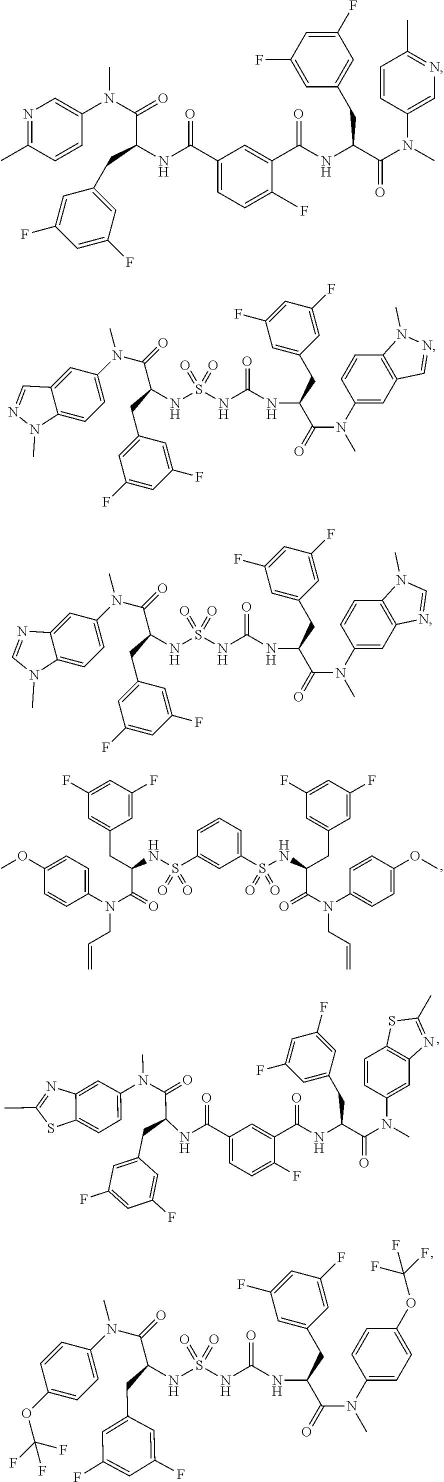

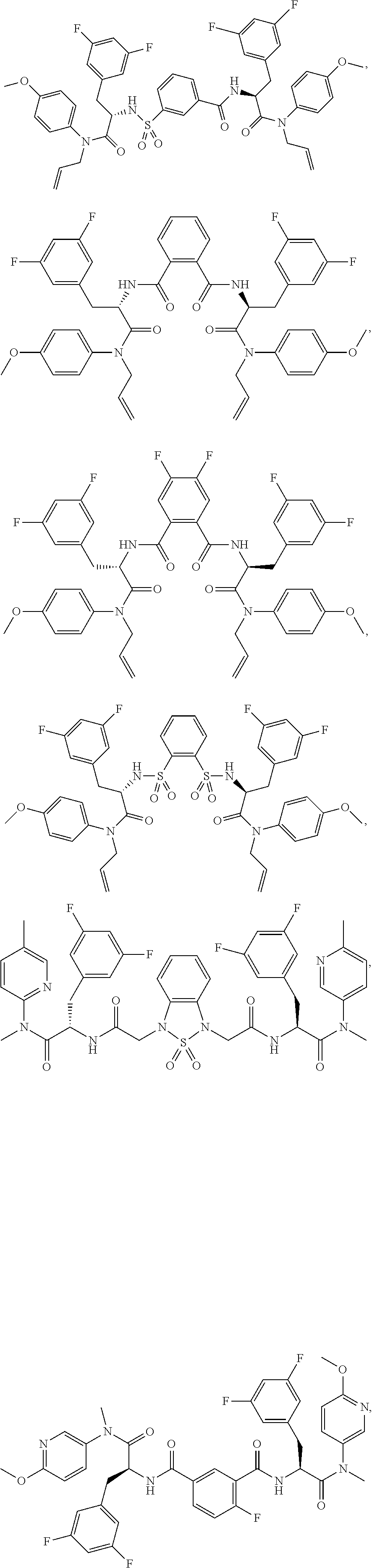

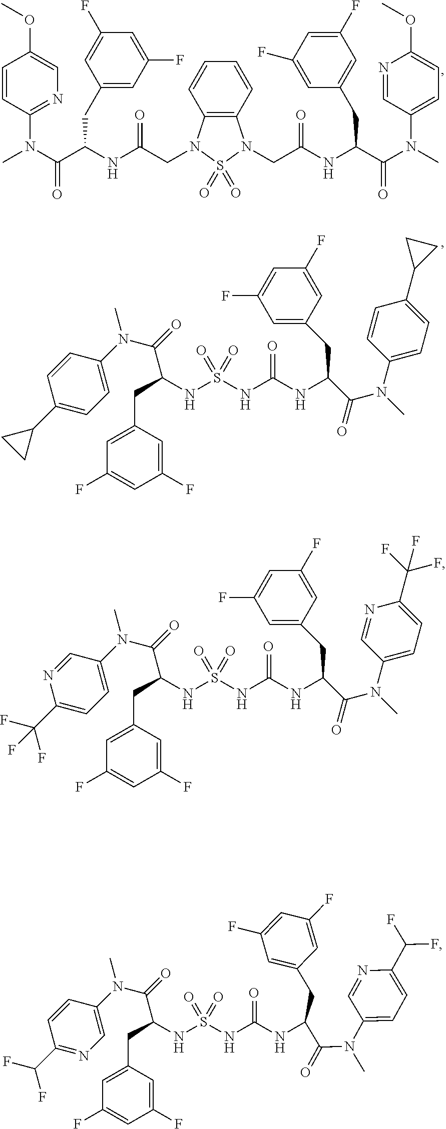 [SMILES: COc1ccc(N(C)C(=O)[C@H](Cc2cc(F)cc(F)c2)NC(=O)CN2c3ccccc3N(CC(=O)N[C@@H](Cc3cc(F)cc(F)c3)C(=O)N(C)c3ccc(OC)nc3)S2(=O)=O)nc1 ; CN(C(=O)[C@H](Cc1cc(F)cc(F)c1)NC(=O)NS(=O)(=O)N[C@@H](Cc1cc(F)cc(F)c1)C(=O)N(C)c1ccc(C2CC2)cc1)c1ccc(C2CC2)cc1 ; CN(C(=O)[C@H](Cc1cc(F)cc(F)c1)NC(=O)NS(=O)(=O)N[C@@H](Cc1cc(F)cc(F)c1)C(=O)N(C)c1ccc(C(F)(F)F)nc1)c1ccc(C(F)(F)F)nc1 ; CN(C(=O)[C@H](Cc1cc(F)cc(F)c1)NC(=O)NS(=O)(=O)N[C@@H](Cc1cc(F)cc(F)c1)C(=O)N(C)c1ccc(C(F)F)nc1)c1ccc(C(F)F)nc1]

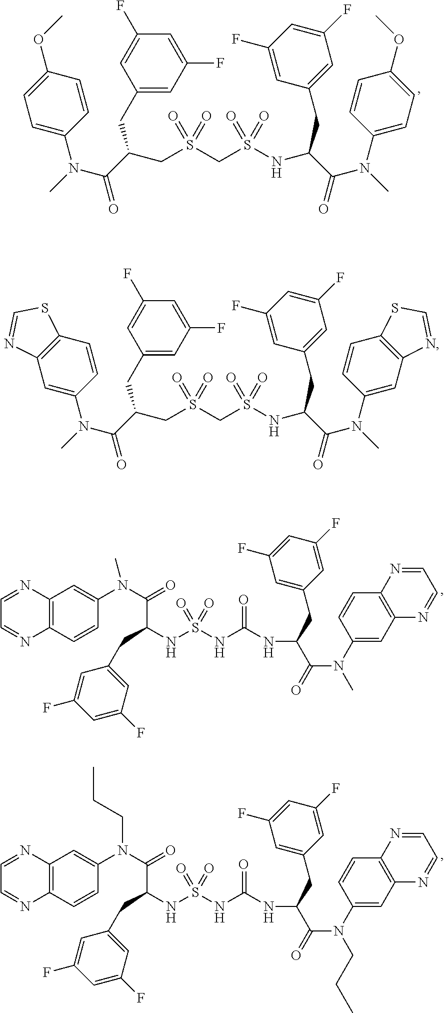 [SMILES: COc1ccc(N(C)C(=O)[C@H](Cc2cc(F)cc(F)c2)CS(=O)(=O)CS(=O)(=O)N[C@@H](Cc2cc(F)cc(F)c2)C(=O)N(C)c2ccc(OC)cc2)cc1 ; CN(C(=O)[C@H](Cc1cc(F)cc(F)c1)CS(=O)(=O)CS(=O)(=O)N[C@@H](Cc1cc(F)cc(F)c1)C(=O)N(C)c1ccc2scnc2c1)c1ccc2scnc2c1 ; CN(C(=O)[C@H](Cc1cc(F)cc(F)c1)NC(=O)NS(=O)(=O)N[C@@H](Cc1cc(F)cc(F)c1)C(=O)N(C)c1ccc2nccnc2c1)c1ccc2nccnc2c1 ; CCCN(C(=O)[C@H](Cc1cc(F)cc(F)c1)NC(=O)NS(=O)(=O)N[C@@H](Cc1cc(F)cc(F)c1)C(=O)N(CCC)c1ccc2nccnc2c1)c1ccc2nccnc2c1]

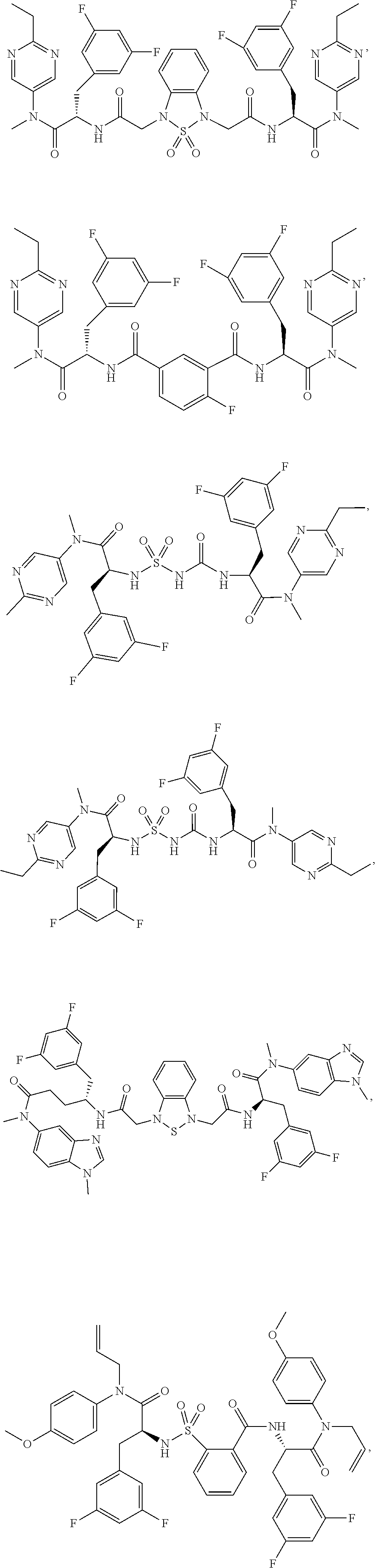

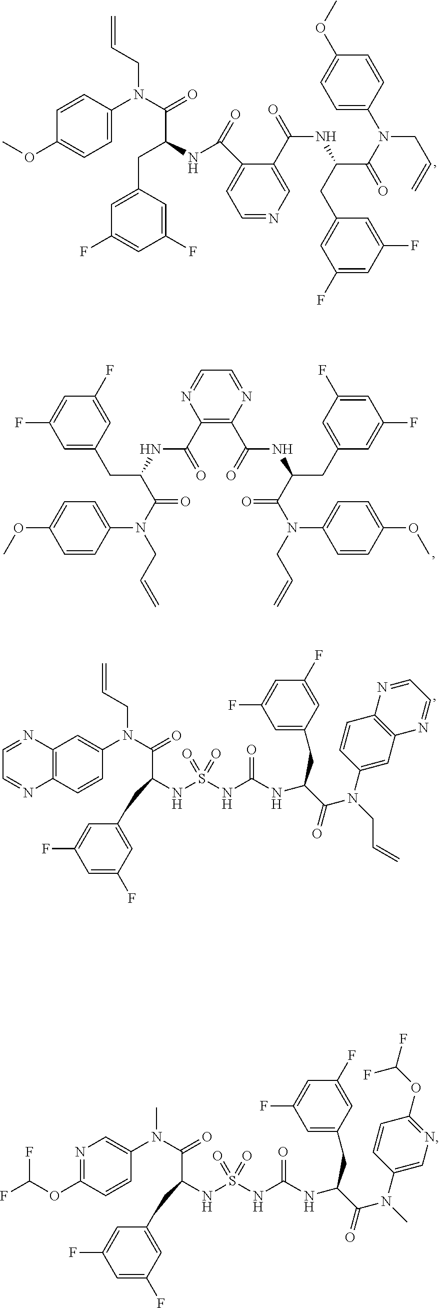

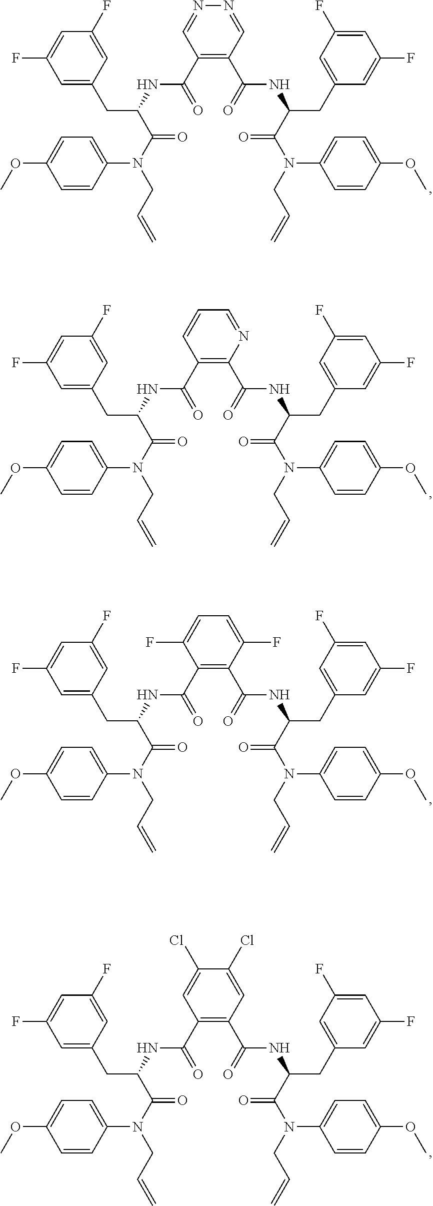

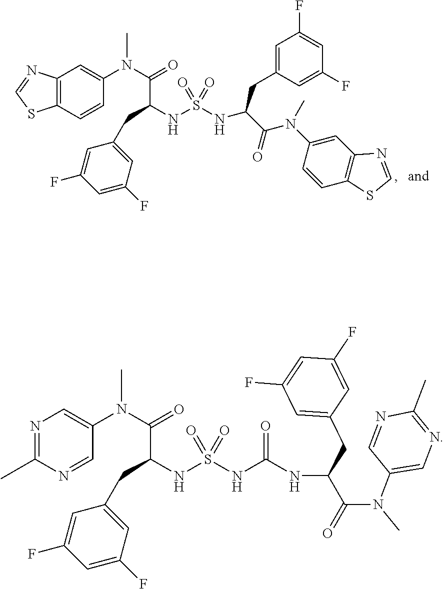 [SMILES: CN(C(=O)[C@H](Cc1cc(F)cc(F)c1)NS(=O)(=O)N[C@@H](Cc1cc(F)cc(F)c1)C(=O)N(C)c1ccc2scnc2c1)c1ccc2scnc2c1 ; Cc1ncc(N(C)C(=O)[C@H](Cc2cc(F)cc(F)c2)NC(=O)NS(=O)(=O)N[C@@H](Cc2cc(F)cc(F)c2)C(=O)N(C)c2cnc(C)nc2)cn1]

### Pharmaceutical Compositions and Methods of Use

The compounds of the invention herein described and set forth are generally given as pharmaceutical compositions. These compositions are comprised of a therapeutically effective amount of a compound of Formulas I-VI or its pharmaceutically acceptable salt, and a pharmaceutically acceptable carrier and may contain conventional excipients and/or diluents. A therapeutically effective amount is that which is needed to provide a meaningful patient benefit. Pharmaceutically acceptable carriers are those conventionally known carriers having acceptable safety profiles. Compositions encompass all common solid and liquid forms, including capsules, tablets, lozenges, and powders, as well as liquid suspensions, syrups, elixirs, and solutions. Compositions are made using available formulation techniques, and excipients (such as binding and wetting agents) and vehicles (such as water and alcohols) which are generally used for compositions. See, for example, Remington's Pharmaceutical Sciences, 17th edition, Mack Publishing Company, Easton, Pa. (1985).

Solid compositions which are normally formulated in dosage units and compositions providing from about 1 to 1000 mg of the active ingredient per dose are preferred. Some examples of dosages are 1 mg, 10 mg, 100 mg, 250 mg, 500 mg, and 1000 mg. Generally, other antiretroviral agents will be present in a unit range similar to agents of that class used clinically. Typically, this is about 0.25-1000 mg/unit.

Liquid compositions are usually in dosage unit ranges. Generally, the liquid composition will be in a unit dosage range of about 1-100 mg/mL. Some examples of dosages are 1 mg/mL, 10 mg/mL, 25 mg/mL, 50 mg/mL, and 100 mg/mL. Generally, other antiretroviral agents will be present in a unit range similar to agents of that class used clinically. Typically, this is about 1-100 mg/mL.

The invention encompasses all conventional modes of administration; oral and parenteral methods are preferred. Generally, the dosing regimen will be similar to other antiretroviral agents used clinically. Typically, the daily dose will be about 1-100 mg/kg body weight daily. Generally, more compound is required orally and less parenterally. The specific dosing regimen, however, will be determined by a physician using sound medical judgment.

The compounds of this invention desireably have activity against HIV. Accordingly, another aspect of the invention is a method for treating HIV infection in a human patient comprising administering a therapeutically effective amount of a compound of Formulas I-VI, including a pharmaceutically acceptable salt thereof, with a pharmaceutically acceptable carrier, excipient and/or diluent.

The invention also encompasses methods where the compound is given in combination therapy. That is, the compound can be used in conjunction with, but separately from, other agents useful in treating AIDS and HIV infection. The compound can also be used in combination therapy wherein the compound and one or more of the other agents are physically together in a fixed-dose combination (FDC). Some of these agents include HIV attachment inhibitors, CCR5 inhibitors, CXCR4 inhibitors, HIV cell fusion inhibitors, HIV integrase inhibitors, HIV nucleoside reverse transcriptase inhibitors, HIV non-nucleoside reverse transcriptase inhibitors, HIV protease inhibitors, budding and maturation inhibitors, immunomodulators, and anti-infectives. In these combination methods, the compound of Formulas I-VI will generally be given in a daily dose of about 1-100 mg/kg body weight daily in conjunction with other agents. The other agents generally will be given in the amounts used therapeutically. The specific dosing regimen, however, will be determined by a physician using sound medical judgment.

“Combination,” “coadministration,” “concurrent” and similar terms referring to the administration of a compound of Formulas I-VI with at least one anti-HIV agent mean that the components are part of a combination antiretroviral therapy or HAART as understood by practitioners in the field of AIDS and HIV infection.

Thus, as set forth above, contemplated herein are combinations of the compounds of Formulas I-VI, together with one or more agents useful in the treatment of AIDS. For example, the compounds of the invention may be effectively administered, whether at periods of pre-exposure and/or post-exposure, in combination with effective amounts of the AIDS antivirals, immunomodulators, anti-infectives, or vaccines, such as those in the following non-limiting table:

| | | |
|---|---|---|
| Drug Name | Manufacturer | Indication |

ANTIVIRALS

| | | |
|---|---|---|
| Rilpivirine | Tibotec | HIV infection, AIDS, ARC |
| | | (non-nucleoside |
| | | reverse transcriptase |
| | | inhibitor) |
| COMPLERA ® | Gilead | HIV infection, AIDS, |
| | | ARC; combination |
| | | with emtricitabine, rilpivirine, |
| | | and tenofovir disoproxil |
| | | fumarate |
| 097 | Hoechst/Bayer | HIV infection, |
| | | AIDS, ARC |
| | | (non-nucleoside |
| | | reverse tran- |
| | | scriptase (RT) |
| | | inhibitor) |
| Amprenavir | Glaxo Wellcome | HIV infection, |
| 141 W94 | | AIDS, ARC |
| GW 141 | | (protease inhibitor) |
| Abacavir (1592U89) | Glaxo Wellcome | HIV infection, |
| GW 1592 | | AIDS, ARC |
| | | (RT inhibitor) |
| Acemannan | Carrington Labs | ARC |
| | (Irving, TX) | |
| Acyclovir | Burroughs Wellcome | HIV infection, AIDS, |
| | | ARC |
| AD-439 | Tanox Biosystems | HIV infection, AIDS, |
| | | ARC |
| AD-519 | Tanox Biosystems | HIV infection, AIDS, |
| | | ARC |
| Adefovir dipivoxil | Gilead Sciences | HIV infection |
| AL-721 | Ethigen | ARC, PGL |
| | (Los Angeles, CA) | HIV positive, AIDS |
| Alpha Interferon | Glaxo Wellcome | Kaposi's sarcoma, |
| | | HIV in combination w/Retrovir |
| Ansamycin | Adria Laboratories | ARC |
| LM 427 | (Dublin, OH) | |
| | Erbamont | |
| | (Stamford, CT) | |
| Antibody which | Advanced Biotherapy | AIDS, ARC |
| Neutralizes pH | Concepts | |
| Labile alpha aberrant | (Rockville, MD) | |
| Interferon | | |
| AR177 | Aronex Pharm | HIV infection, AIDS, |
| | | ARC |
| Beta-fluoro-ddA | Nat'l Cancer Institute | AIDS-associated |
| | | diseases |
| BMS-234475 | Bristol-Myers Squibb/ | HIV infection, |
| (CGP-61755) | Novartis | AIDS, ARC |
| | | (protease inhibitor) |
| CI-1012 | Warner-Lambert | HIV-1 infection |
| Cidofovir | Gilead Science | CMV retinitis, |
| | | herpes, papillomavirus |
| Curdlan sulfate | AJI Pharma USA | HIV infection |
| Cytomegalovirus | MedImmune | CMV retinitis |
| Immune globin | | |
| Cytovene | Syntex | Sight threatening |
| Ganciclovir | | CMV |
| | | peripheral CMV |
| | | retinitis |
| Darunavir | Tibotec- J & J | HIV infection, AIDS, ARC |
| | | (protease inhibitor) |
| Delaviridine | Pharmacia-Upjohn | HIV infection, |
| | | AIDS, ARC |
| | | (RT inhibitor) |
| Dextran Sulfate | Ueno Fine Chem. | AIDS, ARC, HIV |
| | Ind. Ltd. (Osaka, | positive |
| | Japan) | asymptomatic |
| ddC | Hoffman-La Roche | HIV infection, AIDS, |
| Dideoxycytidine | | ARC |
| ddI | Bristol-Myers Squibb | HIV infection, AIDS, |
| Dideoxyinosine | | ARC; combination |
| | | with AZT/d4T |
| DMP-450 | AVID | HIV infection, |
| | (Camden, NJ) | AIDS, ARC |
| | | (protease inhibitor) |
| Efavirenz | Bristol Myers Squibb | HIV infection, |
| (DMP 266, SUSTIVA ®) | | AIDS, ARC |
| (−)6-Chloro-4-(S)- | | (non-nucleoside RT |
| cyclopropylethynyl- | | inhibitor) |
| 4(S)-trifluoro- | | |
| methyl-1,4-dihydro- | | |
| 2H-3,1-benzoxazin- | | |
| 2-one, STOCRINE | | |
| EL10 | Elan Corp, PLC | HIV infection |
| | (Gainesville, GA) | |
| Etravirine | Tibotec/J & J | HIV infection, AIDS, ARC |
| | | (non-nucleoside |
| | | reverse transcriptase |
| | | inhibitor) |
| Famciclovir | Smith Kline | herpes zoster, |
| | | herpes simplex |
| GS 840 | Gilead | HIV infection, |
| | | AIDS, ARC |
| | | (reverse transcriptase |
| | | inhibitor) |
| HBY097 | Hoechst Marion | HIV infection, |
| | Roussel | AIDS, ARC |
| | | (non-nucleoside |
| | | reverse transcriptase |
| | | inhibitor) |
| Hypericin | VIMRx Pharm. | HIV infection, AIDS, |
| | | ARC |
| Recombinant Human | Triton Biosciences | AIDS, Kaposi's |
| Interferon Beta | (Almeda, CA) | sarcoma, ARC |
| Interferon alfa-n3 | Interferon Sciences | ARC, AIDS |
| Indinavir | Merck | HIV infection, AIDS, |
| | | ARC, asymptomatic |
| | | HIV positive, also in |
| | | combination with |
| | | AZT/ddI/ddC |
| ISIS 2922 | ISIS Pharmaceuticals | CMV retinitis |
| KNI-272 | Nat'l Cancer Institute | HIV-assoc. diseases |
| Lamivudine, 3TC | Glaxo Wellcome | HIV infection, |
| | | AIDS, ARC |
| | | (reverse |
| | | transcriptase |
| | | inhibitor); also |
| | | with AZT |
| Lobucavir | Bristol-Myers Squibb | CMV infection |
| Nelfinavir | Agouron | HIV infection, |
| | Pharmaceuticals | AIDS, ARC |
| | | (protease inhibitor) |
| Nevirapine | Boeheringer | HIV infection, |
| | Ingleheim | AIDS, ARC |
| | | (RT inhibitor) |
| Novapren | Novaferon Labs, Inc. | HIV inhibitor |
| | (Akron, OH) | |
| Peptide T | Peninsula Labs | AIDS |
| Octapeptide | (Belmont, CA) | |
| Sequence | | |
| Trisodium | Astra Pharm. | CMV retinitis, HIV |
| Phosphonoformate | Products, Inc. | infection, other CMV |
| | | infections |
| PNU-140690 | Pharmacia Upjohn | HIV infection, |
| | | AIDS, ARC |
| | | (protease inhibitor) |
| Probucol | Vyrex | HIV infection, AIDS |
| RBC-CD4 | Sheffield Med. | HIV infection, |
| | Tech (Houston, TX) | AIDS, ARC |
| Ritonavir | Abbott | HIV infection, |
| | | AIDS, ARC |
| | | (protease inhibitor) |
| Saquinavir | Hoffmann- | HIV infection, |
| | LaRoche | AIDS, ARC |
| | | (protease inhibitor) |
| Stavudine; d4T | Bristol-Myers Squibb | HIV infection, AIDS, |
| Didehydrodeoxy- | | ARC |
| Thymidine | | |
| Tipranavir | Boehringer Ingelheim | HIV infection, AIDS, ARC |
| | | (protease inhibitor) |
| Valaciclovir | Glaxo Wellcome | Genital HSV & CMV |
| | | Infections |
| Virazole | Viratek/ICN | asymptomatic HIV |
| Ribavirin | (Costa Mesa, CA) | positive, LAS, ARC |
| VX-478 | Vertex | HIV infection, AIDS, |
| | | ARC |
| Zalcitabine | Hoffmann-LaRoche | HIV infection, AIDS, |
| | | ARC, with AZT |
| Zidovudine; AZT | Glaxo Wellcome | HIV infection, AIDS, |
| | | ARC, Kaposi's |
| | | sarcoma, in combination with |
| | | other therapies |
| Tenofovir disoproxil, | Gilead | HIV infection, |
| fumarate salt (VIREAD ®) | | AIDS, |
| | | (reverse transcriptase |
| | | inhibitor) |
| EMTRIVA ® (Emtricitabine) | Gilead | HIV infection, |
| (FTC) | | AIDS, |
| | | (reverse transcriptase |
| | | inhibitor) |
| COMBIVIR ® | GSK | HIV infection, |
| | | AIDS, |
| | | (reverse transcriptase |
| | | inhibitor) |
| Abacavir succinate | GSK | HIV infection, |
| (or ZIAGEN ®) | | AIDS, |
| | | (reverse transcriptase |
| | | inhibitor) |
| REYATAZ ® | Bristol-Myers Squibb | HIV infection |
| (or atazanavir) | | AIDs, protease |
| | | inhibitor |
| FUZEON ® | Roche/Trimeris | HIV infection |
| (Enfuvirtide or T-20) | | AIDs, viral Fusion |
| | | inhibitor |
| LEXIVA ® | GSK/Vertex | HIV infection |
| (or Fosamprenavir calcium) | | AIDs, viral protease |
| | | inhibitor |
| SELZENTRY ® | Pfizer | HIV infection |
| Maraviroc; (UK 427857) | | AIDs, (CCR5 antagonist, in |
| | | development) |
| TRIZIVIR ® | GSK | HIV infection |
| | | AIDs, (three drug combination) |
| Sch-417690 (vicriviroc) | Schering-Plough | HIV infection |
| | | AIDs, (CCR5 antagonist, in |
| | | development) |
| TAK-652 | Takeda | HIV infection |
| | | AIDs, (CCR5 antagonist, in |
| | | development) |
| GSK 873140 | GSK/ONO | HIV infection |
| (ONO-4128) | | AIDs, (CCR5 antagonist, |
| | | in development) |
| Integrase Inhibitor | Merck | HIV infection |
| MK-0518 | | AIDs |
| Raltegravir | | |
| TRUVADA ® | Gilead | Combination of Tenofovir |
| | | disoproxil fumarate salt |
| | | (VIREAD ®) and EMTRIVA ® |
| | | (Emtricitabine) |
| Integrase Inhibitor | Gilead/Japan Tobacco | HIV Infection |
| GS917/JTK-303 | | AIDs |
| Elvitegravir | | in development |
| Triple drug combination | Gilead/Bristol-Myers Squibb | Combination of Tenofovir |
| ATRIPLA ® | | disoproxil fumarate salt |
| | | (VIREAD ®), EMTRIVA ® |
| | | (Emtricitabine), and |
| | | SUSTIVA ® (Efavirenz) |
| FESTINAVIR ® | Oncolys BioPharma | HIV infection |
| | | AIDs |
| | | in development |
| CMX-157 | Chimerix | HIV infection |
| Lipid conjugate of | | AIDs |
| nucleotide tenofovir | | |
| GSK1349572 | GSK | HIV infection |
| Integrase inhibitor | | AIDS |

IMMUNOMODULATORS

| | | |
|---|---|---|
| AS-101 | Wyeth-Ayerst | AIDS |
| Bropirimine | Pharmacia Upjohn | Advanced AIDS |
| Acemannan | Carrington Labs, Inc. | AIDS, ARC |
| | (Irving, TX) | |
| CL246,738 | Wyeth | AIDS, Kaposi's |
| | Lederle Labs | sarcoma |
| FP-21399 | Fuki ImmunoPharm | Blocks HIV fusion |
| | | with CD4+ cells |
| Gamma Interferon | Genentech | ARC, in combination |
| | | w/TNF (tumor |
| | | necrosis factor) |
| Granulocyte | Genetics Institute | AIDS |
| Macrophage Colony | Sandoz | |
| Stimulating Factor | | |
| Granulocyte | Hoechst-Roussel | AIDS |
| Macrophage Colony | Immunex | |
| Stimulating Factor | | |
| Granulocyte | Schering-Plough | AIDS, |
| Macrophage Colony | | combination |
| Stimulating Factor | | w/AZT |
| HIV Core Particle | Rorer | Seropositive HIV |
| Immunostimulant | | |
| IL-2 | Cetus | AIDS, in combination |
| Interleukin-2 | | w/AZT |
| IL-2 | Hoffman-LaRoche | AIDS, ARC, HIV, in |
| Interleukin-2 | Immunex | combination w/AZT |
| IL-2 | Chiron | AIDS, increase in |
| Interleukin-2 | | CD4 cell counts |
| (aldeslukin) | | |
| Immune Globulin | Cutter Biological | Pediatric AIDS, in |
| Intravenous | (Berkeley, CA) | combination w/AZT |
| (human) | | |
| IMREG-1 | Imreg | AIDS, Kaposi's |
| | (New Orleans, LA) | sarcoma, ARC, PGL |
| IMREG-2 | Imreg | AIDS, Kaposi's |
| | (New Orleans, LA) | sarcoma, ARC, PGL |
| Imuthiol Diethyl | Merieux Institute | AIDS, ARC |
| Dithio Carbamate | | |
| Alpha-2 | Schering Plough | Kaposi's sarcoma |
| Interferon | | w/AZT, AIDS |
| Methionine- | TNI Pharmaceutical | AIDS, ARC |
| Enkephalin | (Chicago, IL) | |
| MTP-PE | Ciba-Geigy Corp. | Kaposi's sarcoma |
| Muramyl-Tripeptide | | |
| Granulocyte | Amgen | AIDS, in combination |
| Colony Stimulating | | w/AZT |
| Factor | | |
| Remune | Immune Response | Immunotherapeutic |
| | Corp. | |
| rCD4 | Genentech | AIDS, ARC |
| Recombinant | | |
| Soluble Human CD4 | | |
| rCD4-IgG | | AIDS, ARC |
| hybrids | | |
| Recombinant | Biogen | AIDS, ARC |
| Soluble Human CD4 | | |
| Interferon | Hoffman-La Roche | Kaposi's sarcoma |
| Alfa 2a | | AIDS, ARC, |
| | | in combination w/AZT |
| SK&F106528 | Smith Kline | HIV infection |
| Soluble T4 | | |
| Thymopentin | Immunobiology | HIV infection |
| | Research Institute | |
| | (Annandale, NJ) | |
| Tumor Necrosis | Genentech | ARC, in combination |
| Factor; TNF | | w/gamma Interferon |

ANTI-INFECTIVES

| | | |
|---|---|---|
| Clindamycin with | Pharmacia Upjohn | PCP |
| Primaquine | | |
| Fluconazole | Pfizer | Cryptococcal |
| | | meningitis, |
| | | candidiasis |
| Pastille | Squibb Corp. | Prevention of |
| Nystatin Pastille | | oral candidiasis |
| Ornidyl | Merrell Dow | PCP |
| Eflornithine | | |
| Pentamidine | LyphoMed | PCP treatment |
| Isethionate (IM & IV) | (Rosemont, IL) | |
| Trimethoprim | | Antibacterial |
| Trimethoprim/sulfa | | Antibacterial |
| Piritrexim | Burroughs Wellcome | PCP treatment |
| Pentamidine | Fisons Corporation | PCP prophylaxis |
| Isethionate for | | |
| Inhalation | | |
| Spiramycin | Rhone-Poulenc | Cryptosporidial |
| | diarrhea | |
| Intraconazole- | Janssen-Pharm. | Histoplasmosis; |
| R51211 | | cryptococcal |
| | | meningitis |
| Trimetrexate | Warner-Lambert | PCP |
| Daunorubicin | NeXstar, Sequus | Kaposi's sarcoma |
| Recombinant Human | Ortho Pharm. Corp. | Severe anemia |
| Erythropoietin | | assoc. with AZT |
| | | therapy |
| Recombinant Human | Serono | AIDS-related |
| Growth Hormone | | wasting, cachexia |
| Megestrol Acetate | Bristol-Myers Squibb | Treatment of |
| | | anorexia assoc. |
| | | W/AIDS |
| Testosterone | Alza, Smith Kline | AIDS-related wasting |
| Total Enteral | Norwich Eaton | Diarrhea and |
| Nutrition | Pharmaceuticals | malabsorption |
| | | related to AIDS |

“Therapeutically effective” means the amount of agent required to provide a meaningful patient benefit as understood by practitioners in the field of AIDS and HIV infection. In general, the goals of therapeutically effective treatment include suppression of viral load, restoration and preservation of immunologic function, improved quality of life, and reduction of HIV-related morbidity and mortality.

“Patient” means a person infected with the HIV virus and suitable for therapy as understood by practitioners in the field of AIDS and HIV infection.

“Treatment,” “therapy,” “regimen,” “HIV infection,” “ARC,” “AIDS” and related terms are used as understood by practitioners in the field of AIDS and HIV infection.

### Methods of Synthesis

The compounds of the invention according to the various aspects can be made by various methods available in the art, including those of the following schemes in the specific examples which follow. The starting materials suitable for use in making the compounds of the invention are readily commercially available or can be readily prepared by those skilled in the art. The structure numbering and variable numbering shown in the synthetic schemes may be distinct from, and should not be confused with, the structure or variable numbering in the claims or the rest of the specification. The variables in the schemes are meant only to illustrate how to make some of the compounds of the invention.

Abbreviations used in the schemes generally follow conventions used in the art. Some specific chemical abbreviations used in the examples are defined as follows: “DMF” for N,N-dimethylformamide; “MeOH” for methanol; “Ar” for aryl; “TFA” for trifluoroacetic acid; “BOC” for t-butoxycarbonate, “DMSO” for dimethylsulfoxide; “h” for hours; “rt” for room temperature or retention time (context will dictate); “min” for minutes; “EtOAc” for ethyl acetate; “THF” for tetrahydrofuran; “Et₂O” for diethyl ether; “DMAP” for 4-dimethylaminopyridine; “DCE” for 1,2-dichloroethane; “ACN” for acetonitrile; “DME” for 1,2-dimethoxyethane; “HATU” for (1-[Bis(dimethylamino)methylene]-1H-1,2,3-triazolo[4,5-b]pyridinium 3-oxid hexafluorophosphate) “DIEA” for diisopropylethylamine.

Certain other abbreviations as used herein, are defined as follows: “1×” for once, “2×” for twice, “3×” for thrice, “° C.” for degrees Celsius, “eq” for equivalent or equivalents, “g” for gram or grams, “mg” for milligram or milligrams, “L” for liter or liters, “mL” for milliliter or milliliters, “μL” for microliter or microliters, “N” for normal, “M” for molar, “mmol” for millimole or millimoles, “min” for minute or minutes, “h” for hour or hours, “rt” for room temperature, “RT” for retention time, “atm” for atmosphere, “psi” for pounds per square inch, “conc.” for concentrate, “sat” or “sat'd ” for saturated, “MW” for molecular weight, “mp” for melting point, “ee” for enantiomeric excess, “MS” or “Mass Spec” for mass spectrometry, “ESI” for electrospray ionization mass spectroscopy, “HR” for high resolution, “HRMS” for high resolution mass spectrometry, “LCMS” for liquid chromatography mass spectrometry, “HPLC” for high pressure liquid chromatography, “RP HPLC” for reverse phase HPLC, “TLC” or “tic” for thin layer chromatography, “NMR” for nuclear magnetic resonance spectroscopy, “¹H” for proton, “δ” for delta, “s” for singlet, “d” for doublet, “t” for triplet, “q” for quartet, “m” for multiplet, “br” for broad, “Hz” for hertz, and “α”, “β”, “R”, “S”, “E”, and “Z” are stereochemical designations familiar to one skilled in the art.

### EXAMPLES

The following examples are provided by way of illustration only, and should not be construed as limiting the scope of the invention. The 2,3-dihydro-1H-pyrrolo[2,3-b]pyridine reactant noted in certain experimentals, such as in Example 151, is not required for the preparation of said Example but was included, as a matter of convenience, for the purpose of preparing in the same reaction pot an alternate set of analogs not required for the synthesis of the compounds of this invention.

### Intermediate 1

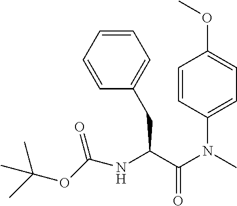 [SMILES: COc1ccc(N(C)C(=O)[C@H](Cc2ccccc2)NC(=O)OC(C)(C)C)cc1]

HATU (1.52 g, 4.01 mmol) was added to a stirred solution of 4-methoxy-N-methylaniline (500 mg, 3.64 mmol) and (S)-2-((tert-butoxycarbonyl)amino)-3-phenylpropanoic acid (1.06 g, 4.01 mmol) iIn DMF (20 mL) and DIPEA (1.27 mL, 7.29 mmol) and the reaction mixture was stirred at rt for 4 h. The reaction mixture was concentrated and the crude oil was then partitioned between EtOAc (˜60 mL) and ½ sat. NaHCO₃ (aq.) (˜60 mL). The organic component was washed with brine (˜40 mL), dried (MgSO₄), filtered and concentrated. The residual oil was then purified using a Biotage Horizon (80 g SiO₂, 10-40% EtOAc/hexanes) to yield Intermediate 1 (1.34 g) as clear amber viscous oil. LC-MS retention time=3.17 min; m/z=285.3 [M+H-Boc]⁺. (Column: Phenomenex Luna C18 2.0×50 mm 3 μm. Solvent A=95% Water: 5% Acetonitrile: 10 mM NH₄OAc. Solvent B=5% Water: 95% Acetonitrile: 10 mM NH₄OAc. Flow Rate=0.8 mL/min. Start % B=0. Final % B=100. Gradient Time=4 minutes, then a 1-minute hold at 100% B. Wavelength=220 nm). ¹H NMR (400 MHZ, CDCl₃) δ ppm 7.25-7.20 (m, 3H), 7.03-6.64 (m, 6H), 5.20 (d, J=8.8 Hz, 1H), 4.53 (app q, J=7.4 Hz, 1H), 3.83 (s, 3H), 3.18 (s, 3H), 2.89 (dd, J=13.1, 7.5 Hz, 1H), 2.71 (dd, J=13.1, 6.5 Hz, 1H), 1.39 (s, 9H).

### Intermediate 2

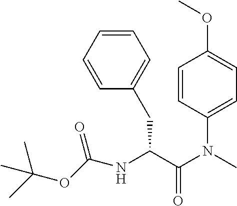 [SMILES: COc1ccc(N(C)C(=O)[C@@H](Cc2ccccc2)NC(=O)OC(C)(C)C)cc1]

Compound was prepared using the procedure described for Intermediate 1 where (R)-2-((tert-butoxycarbonyl)amino)-3-phenylpropanoic acid was used in place of (S)-2-((tert-butoxycarbonyl)-amino)-3-phenylpropanoic acid. LC-MS retention time=1.74 min; m/z=407.4 [M+Na]⁺. (Column: Phenomenex Luna C18 30×2.0 mm 3 μm. Solvent A=90% Water: 10% Acetonitrile: 0.1% TFA. Solvent B=10% Water: 90% Acetonitrile: 0.1% TFA. Flow Rate=1 mL/min. Start % B=0. Final % B=100. Gradient Time=2 minutes, then a 1-minute hold at 100% B. Wavelength=220 nm). ¹H NMR (400 MHZ, CDCl₃) δ ppm 7.25-7.20 (m, 3H), 7.00-6.66 (m, 6H), 5.20 (d, J=9.0 Hz, 1H), 4.58-4.48 (m, 1H), 3.82 (s, 3H), 3.18 (s, 3H), 2.89 (dd, J=13.2, 7.4 Hz, 1H), 2.71 (dd, J=13.1, 6.8 Hz, 1H), 1.39 (s, 9H).

### Intermediate 3

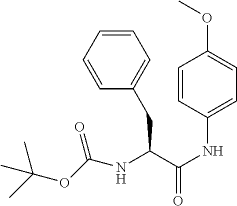 [SMILES: COc1ccc(NC(=O)[C@H](Cc2ccccc2)NC(=O)OC(C)(C)C)cc1]

Compound was prepared using the procedure described for Intermediate 1 where 4-methoxyaniline was used in place of 4-methoxy-N-methylaniline. LC-MS retention time=1.71 min; m/z=393.4 [M+Na]⁺. (Column: Phenomenex Luna C18 30×2.0 mm 3 μm. Solvent A=90% Water: 10% Acetonitrile: 0.1% TFA. Solvent B=10% Water: 90% Acetonitrile: 0.1% TFA. Flow Rate=1 mL/min. Start % B=0. Final % B=100. Gradient Time=2 minutes, then a 1-minute hold at 100% B. Wavelength=220 nm). ¹H NMR (400 MHZ, CDCl₃) δ ppm 7.68 (br. s., 1H), 7.35-7.22 (m, 7H), 6.82 (d, J=9.0 Hz, 2H), 5.24 (br. s., 1H), 4.48 (br. s., 1H), 3.78 (s, 3H), 3.15 (d, J=6.8 Hz, 2H), 1.43 (s, 9H).

### Intermediate 4

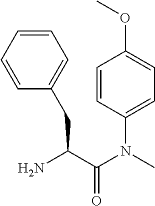 [SMILES: COc1ccc(N(C)C(=O)[C@@H](N)Cc2ccccc2)cc1]

A solution of 4M HCl (15 mL, 60 mmol) in 1,4-dioxane was added to a stirred solution of Intermediate 1 (1.34 g, 3.49 mmol) in THF (10 mL) and the reaction mixture was stirred at rt for 5 h. The reaction mixture was concentrated to yield an HCl salt of Intermediate 4 (1.11 g) which was used without additional purification. LC-MS retention time=2.33 min; m/z=285.2 [M+H]⁺. (Column: Phenomenex Luna C18 2.0×50 mm 3 μm. Solvent A=95% Water: 5% Acetonitrile: 10 mM NH₄OAc. Solvent B=5% Water: 95% Acetonitrile: 10 mM NH₄OAc. Flow Rate=0.8 mL/min. Start % B=0. Final % B=100. Gradient Time=4 minutes, then a 1-minute hold at 100% B. Wavelength=220 nm).

### Intermediate 5

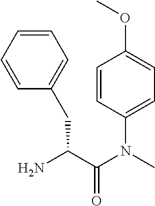 [SMILES: COc1ccc(N(C)C(=O)[C@H](N)Cc2ccccc2)cc1]

Compound was prepared using the procedure described for Intermediate 4 where Intermediate 2 was used in place of Intermediate 1. LC-MS retention time=1.15 min; m/z=285.3 [M+H]⁺. (Column: Phenomenex Luna C18 30×2.0 mm 3 μm. Solvent A=90% Water: 10% Acetonitrile: 0.1% TFA. Solvent B=10% Water: 90% Acetonitrile: 0.1% TFA. Flow Rate=1 mL/min. Start % B=0. Final % B=100. Gradient Time=2 minutes, then a 1-minute hold at 100% B. Wavelength=220 nm).

### Intermediate 6

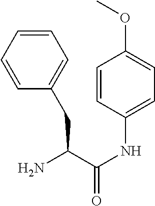 [SMILES: COc1ccc(NC(=O)[C@@H](N)Cc2ccccc2)cc1]

Compound was prepared using the procedure described for Intermediate 4 where Intermediate 3 was used in place of Intermediate 1. LC-MS retention time=1.12 min; m/z=271.3 [M+H]⁺. (Column: Phenomenex Luna C18 30×2.0 mm 3 μm. Solvent A=90% Water: 10% Acetonitrile: 0.1% TFA. Solvent B=10% Water: 90% Acetonitrile: 0.1% TFA. Flow Rate=1 mL/min. Start % B=0. Final % B=100. Gradient Time=2 minutes, then a 1-minute hold at 100% B. Wavelength=220 nm).

### Intermediate 7

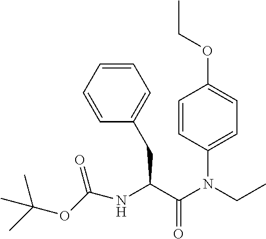 [SMILES: CCOc1ccc(N(CC)C(=O)[C@H](Cc2ccccc2)NC(=O)OC(C)(C)C)cc1]

HATU (725 mg, 1.91 mmol) was added to a stirred solution of 4-ethoxy-N-ethylaniline (300 mg, 1.82 mmol) and (S)-2-((tert-butoxycarbonyl)amino)-3-phenylpropanoic acid (506 mg, 1.91 mmol) in DMF (10 mL) and DIPEA (0.63 mL, 3.6 mmol) and the reaction mixture was stirred at rt for 5 h. The reaction mixture was concentrated and the crude oil was then partitioned between EtOAc (˜60 mL) and sat. NaHCO₃ (aq) (˜50 mL). The organic component was washed with brine (˜50 mL), dried (MgSO₄), filtered and concentrated. The residual oil was purified using a Biotage Horizon (40 g SiO₂, 15-40% EtOAc/hexanes) to yield Intermediate 7 (632 mg) as a solidified off-white foam. LC-MS retention time=1.75 min; m/z=413.3 [M+H]⁺. (Column: Phenomenex Luna 30×2.0 mm 3 μm. Solvent A=90% Water: 10% Acetonitrile: 0.1% TFA. Solvent B=10% Water: 90% Acetonitrile: 0.1% TFA. Flow Rate=1 mL/min. Start % B=0. Final % B=100. Gradient Time=2 minutes, then a 1-minute hold at 100% B. Wavelength=220 nm). ¹H NMR (400 MHZ, CDCl₃) δ ppm 7.26-7.20 (m, 4H), 7.05-6.69 (m, 5H), 5.19 (d, J=8.3 Hz, 1H), 4.47-4.38 (m, 1H), 4.10-3.98 (m, 2H), 3.75 (dq, J=13.6, 7.0 Hz, 1H), 3.61-3.48 (m, 1H), 2.91 (dd, J=13.2, 7.4 Hz, 1H), 2.71 (dd, J=12.5, 6.5 Hz, 1H), 1.44 (t, J=7.0 Hz, 3H), 1.38 (s, 9H), 1.05 (t, J=7.2 Hz, 3H)

### Intermediate 8

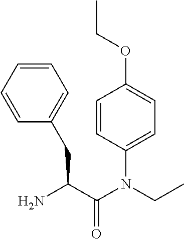 [SMILES: CCOc1ccc(N(CC)C(=O)[C@@H](N)Cc2ccccc2)cc1]

A solution of 4M HCl (1.04 mL, 4.15 mmol) in 1,4-dioxane was added to a stirred solution of Intermediate 7 (684 mg, 1.66 mmol) in THF (2.2 mL) and the reaction mixture was stirred at rt for 5 h. Additional 4M HCl in 1,4-dioxane (1.5 mL) was added and stirring continued overnight. The reaction mixture was concentrated under high vacuum to yield an HCl salt of Intermediate 8 (632 mg) as a solidified foam which was used without further purification. LC-MS retention time=1.24 min; m/z=625.5 [2M+H]⁺. (Column: Phenomenex Luna 30×2.0 mm 3 μm. Solvent A=90% Water: 10% Acetonitrile: 0.1% TFA. Solvent B=10% Water: 90% Acetonitrile: 0.1% TFA. Flow Rate=1 mL/min. Start % B=0. Final % B=100. Gradient Time=2 minutes, then a 1-minute hold at 100% B. Wavelength=220 nm).

### Intermediate 9

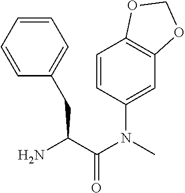 [SMILES: CN(C(=O)[C@@H](N)Cc1ccccc1)c1ccc2c(c1)OCO2]

Compound was prepared using the procedures described for the preparation of Intermediate 4 where 4-methoxy-N-methylaniline was replaced with N-methylbenzo[d][1,3]dioxo1-5-amine. LC-MS retention time=1.16 min; m/z=299.3 [2M+H]⁺. (Column: Phenomenex Luna 30×2.0 mm 3 μm. Solvent A=90% Water: 10% Acetonitrile: 0.1% TFA. Solvent B=10% Water: 90% Acetonitrile: 0.1% TFA. Flow Rate=1 mL/min. Start % B=0. Final % B=100. Gradient Time=2 minutes, then a 1-minute hold at 100% B. Wavelength=220 nm).

### Intermediate 10

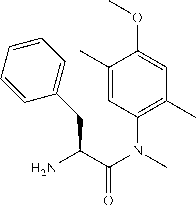 [SMILES: COc1cc(C)c(N(C)C(=O)[C@@H](N)Cc2ccccc2)cc1C]

Compound was prepared using the procedures described for the preparation of Intermediate 4 where 4-methoxy-N-methylaniline was replaced with 2,5-dimethyl-4-methoxy-N-methylaniline. LC-MS retention time=1.29 min; m/z=33.4 [M+H]⁺. (Column: Phenomenex Luna 30×2.0 mm 3 μm. Solvent A=90% Water: 10% Acetonitrile: 0.1% TFA. Solvent B=10% Water: 90% Acetonitrile: 0.1% TFA. Flow Rate=1 mL/min. Start % B=0. Final % B=100. Gradient Time=2 minutes, then a 1-minute hold at 100% B. Wavelength=220 nm).

### Intermediate 11

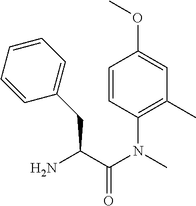 [SMILES: COc1ccc(N(C)C(=O)[C@@H](N)Cc2ccccc2)c(C)c1]

Compound was prepared using the procedures described for the preparation of Intermediate 4 where 4-methoxy-N-methylaniline was replaced with 4-methoxy-2-methyl-N-methylaniline. LC-MS retention time=1.19 min; m/z=299.4 [M+H]⁺. (Column: Phenomenex Luna 30×2.0 mm 3 μm. Solvent A=90% Water: 10% Acetonitrile: 0.1% TFA. Solvent B=10% Water: 90% Acetonitrile: 0.1% TFA. Flow Rate=1 mL/min. Start % B=0. Final % B=100. Gradient Time=2 minutes, then a 1-minute hold at 100% B. Wavelength=220 nm).

### Intermediate 12

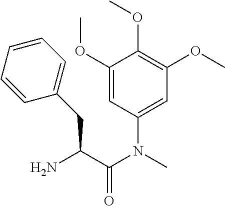 [SMILES: COc1cc(N(C)C(=O)[C@@H](N)Cc2ccccc2)cc(OC)c1OC]

Compound was prepared using the procedures described for the preparation of Intermediate 4 where 4-methoxy-N-methylaniline was replaced with 3,4,5-trimethoxy-N-methylaniline. LC-MS retention time=1.16 min; m/z=345.4 [M+H]⁺. (Column: Phenomenex Luna 30×2.0 mm 3 μm. Solvent A=90% Water: 10% Acetonitrile: 0.1% TFA. Solvent B=10% Water: 90% Acetonitrile: 0.1% TFA. Flow Rate=1 mL/min. Start % B=0. Final % B=100. Gradient Time=2 minutes, then a 1-minute hold at 100% B. Wavelength=220 nm).

### Intermediate 13

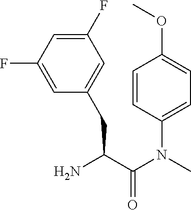 [SMILES: COc1ccc(N(C)C(=O)[C@@H](N)Cc2cc(F)cc(F)c2)cc1]

Compound was prepared using the procedures described for the preparation of Intermediate 4 where (S)-2-((tert-butoxycarbonyl)amino)-3-phenylpropanoic acid was replaced with (S)-2-((tert-butoxycarbonyl)amino)-3-(3,5-difluorophenyl)propanoic acid. LC-MS retention time=2.98 min; m/z=321.1 [M+H]⁺. (Column: Phenomenex Luna 50×2.0 mm 3 μm. Solvent A=90% Water: 10% MeOH: 0.1% TFA. Solvent B=10% Water: 90% MeOH: 0.1% TFA. Flow Rate=0.8 mL/min. Start % B=0. Final % B=100. Gradient Time=4 minutes, then a 1-minute hold at 100% B. Wavelength=220 nm). ¹H NMR (400 MHZ, DMSO-d₆) δ ppm 8.41 (br. s., 3H), 7.21-7.05 (m, 3H), 6.98 (d, J=8.8 Hz, 2H), 6.57 (d, J=6.3 Hz, 2H), 3.90 (br. s., 1H), 3.79 (s, 3H), 3.15 (s, 3H), 3.00-2.92 (m, 1H), 2.90-2.83 (m, 1H) (HCl salt).

### Intermediate 14

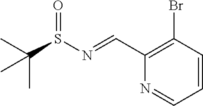

Cupric sulfate (5.49 g, 34.4 mmol) was added to a stirred solution of 3-bromopicolinaldehyde (3.2 g, 17 mmol) and (R)-2-methylpropane-2-sulfinamide (2.28 g, 18.8 mmol) in DCM (40 mL) and the reaction mixture was stirred at rt for 5 h. The reaction mixture was filtered, concentrated and purified by Biotage (15-50% EtOAc/hexanes, 80 g SiO_{2,} 30% EtOAc/Hexanes) to give Intermediate 14 (3.40 g). ¹H NMR (400 MHZ, CDCl₃) δ ppm 9.06 (s, 1H), 8.75 (dd, J=4.5, 1.0 Hz, 1H), 8.02 (dd, J=8.0, 1.3 Hz, 1H), 7.30 (dd, J=8.0, 4.5 Hz, 1H), 1.33 (s, 9H).

### Intermediate 15

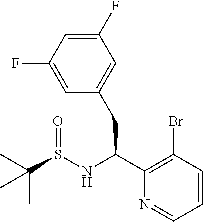 [SMILES: CC(C)(C)[S@@](=O)N[C@@H](Cc1cc(F)cc(F)c1)c1ncccc1Br]

A 0.25 M solution of 3,5-difluorobenzylmagnesium bromide (44.6 mL, 11.2 mmol) was added dropwise (over 30 min) to a solution of Intermediate 14 (2.69 g, 9.30 mmol) in DCM (200 mL) at −78° C. The reaction mixture was stirred at −78° C. for 3 hours and then quenched with sat. aq. NH₄Cl (20 mL) and allowed to warm to rt. The components were separated and the aqueous component was further extracted with EtOAc (2×40 mL). The combined organic component was dried over Na₂SO₄, filtered and concentrated. The crude product was purified by Biotage (Silica 120 gram flash column, EtOAc/hexanes gradient 20-70% EtOAc, 50% EtOAc) to give the title compound (1.81 g). LC-MS retention time=1.69 min; m/z=417.1 [M+H]⁺. (Column: Waters Aquity BEH C18 2.1×50 mm 1.7 μm. Solvent A=90% Water: 10% Acetonitrile: 0.05% TFA. Solvent B=10% Water: 90% Acetonitrile: 0.05% TFA. Flow Rate=0.8 mL/min. Start % B=2. Final % B=98. Gradient Time=1.5 minutes, then a 0.5-minute hold at 98% B. Wavelength=220 nm). ¹H NMR (400 MHZ, CDCl₃) δ ppm 8.51 (dd, J=4.6, 1.1 Hz, 1H), 7.86-7.80 (m, 1H), 7.12 (dd, J=8.2, 4.6 Hz, 1H), 6.65-6.53 (m, 3H), 5.25-5.17 (m, 1H), 4.69-4.47 (m, 1H), 3.37-3.27 (m, 2H), 1.15 (s, 9H).

### Intermediate 16

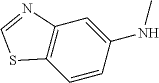

Paraformaldehyde (80 mg, 2.7 mmol) was added to a stirred solution of benzo[d]thiazol-5-amine (200 mg, 1.332 mmol) in MeOH (5 mL). The resulting suspension was then treated with 25% w/w NaOMe in MeOH (1.5 mL, 6.7 mmol) and the clear reaction mixture was stirred at 60° C. for 16 h. The reaction was allowed to cool to rt and then treated with NaBH₄ (126 mg, 3.33 mmol) and stirred at rt for 16 h. The reaction mixture was diluted with water (10 mL) and extracted with CHCl₃ (3×20 mL). The combined organic component was concentrated and purified using a Biotage Horizon (12 g SiO₂, 0-50% EtOAc/hexanes) to yield Intermediate 16 (217 mg) as yellow gum. LC-MS retention time=0.67 min; m/z=165.1 [M+H]⁺. (Column: Waters Aquity BEH C18, 2.0×50 mm, 1.7-μm particles. Solvent A=100% Water: 0.05% TFA. Solvent B=100% Acetonitrile: 0.05% TFA. Flow Rate=0.8 mL/min. Start % B=2. Final % B=98. Gradient Time=1.5 min. Wavelength=220 nm). ¹H NMR (400 MHZ, CDCl₃) δ ppm 8.92 (s, 1H), 7.69 (d, J=8.5 Hz, 1H), 7.31 (d, J=2.3 Hz, 1H), 6.82 (dd, J=8.8, 2.3 Hz, 1H), 3.93 (br. s., 1H), 2.94 (s, 3H).

### Intermediate 17

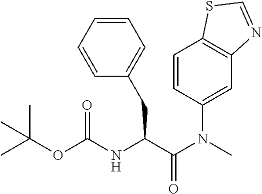 [SMILES: CN(C(=O)[C@H](Cc1ccccc1)NC(=O)OC(C)(C)C)c1ccc2scnc2c1]

HATU (1.90 g, 5.01 mmol) was added to a solution of Intermediate 16 (685 mg, 4.17 mmol) and (S)-2-((tert-butoxycarbonyl)amino)-3-phenylpropanoic acid (1.33 g, 5.01 mmol) in DMF (20 mL) and DIPEA (2.18 mL, 12.5 mmol) and the reaction mixture was stirred at rt for 6 h. The crude reaction mixture was diluted with sat. aq. NaHCO₃ (20 mL) and extracted with EtOAc (3×50 mL). The combined organic component was washed with brine (˜60 mL), dried (Na₂SO₄), filtered and concentrated. The crude material was then purified using a Biotage Horizon (12 g SiO₂, 0-40%-50% EtOAc/hexanes) to yield Intermediate 17 (1.7 g) as a white solid. LC-MS retention time=1.19 min; m/z=412.0 [M+H]⁺. (Column: Waters Aquity BEH C18, 2.0×50 mm, 1.7-μm particles. Solvent A=100% Water: 0.05% TFA. Solvent B=100% Acetonitrile: 0.05% TFA. Flow Rate=0.8 mL/min. Start % B=2. Final % B=98. Gradient Time=1.5 min. Wavelength=220 nm). ¹H NMR (400 MHZ, CDCl₃) δ ppm 9.07 (s, 1H), 7.90 (d, J=8.3 Hz, 1H), 7.38 (d, J=7.5 Hz, 1H), 7.27-7.19 (m, 3H), 6.94 (d, J=6.8 Hz, 3H), 5.22 (d, J=8.8 Hz, 1H), 4.58-4.48 (m, 1H), 3.26 (s, 3H), 2.93 (dd, J=12.9, 8.4 Hz, 1H), 2.78 (dd, J=12.4, 5.9 Hz, 1H), 1.40 (s, 9H).

### Intermediate 18

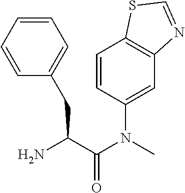 [SMILES: CN(C(=O)[C@@H](N)Cc1ccccc1)c1ccc2scnc2c1]

A solution of 4M HCl (10 mL, 40.0 mmol) in 1,4-dioxane was added to a stirred solution of Intermediate 17 (1.7 g, 4.1 mmol) in THF (10 mL) and the reaction mixture was stirred at rt for 16 h. The reaction mixture was concentrated, redissolved in EtOH/toluene, and then reconcentrated (3×) to yield an HCl salt of Intermediate 18 (1.7 g) as a pink sticky solid. LC-MS retention time=0.83 min; m/z=312.0 [M+H]⁺. (Column: Waters Aquity BEH C18, 2.0×50 mm, 1.7-μm particles. Solvent A=100% Water: 0.05% TFA. Solvent B=100% Acetonitrile: 0.05% TFA. Flow Rate=0.8 mL/min. Start % B=2. Final % B=98. Gradient Time=1.5 min. Wavelength=220 nm). ¹H NMR (400 MHZ, methanol-d₄) δ ppm 9.42 (s, 1H), 8.10 (d, J=8.3 Hz, 1H), 7.39-7.08 (m, 6H), 6.91 (d, J=7.0 Hz, 2H), 4.10 (dd, J=8.0, 6.5 Hz, 1H), 3.63-3.56 (m, 2H), 3.11 (dd, J=13.4, 8.2 Hz, 1H), 2.92 (dd, J=13.3, 6.5 Hz, 1H), 2.87 (s, 3H).

### Intermediate 19

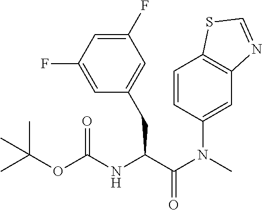 [SMILES: CN(C(=O)[C@H](Cc1cc(F)cc(F)c1)NC(=O)OC(C)(C)C)c1ccc2scnc2c1]

HATU (592 mg, 1.556 mmol) was added to a stirred solution of Intermediate 16 (213 mg, 1.30 mmol) and (S)-2-((tert-butoxycarbonyl)amino)-3-(3,5-difluorophenyl)propanoic acid (469 mg, 1.56 mmol) in DMF (7 mL) and DIPEA (0.45 mL, 2.6 mmol) and the reaction mixture was stirred at rt for 16 h. The crude reaction mixture was diluted with sat. aq. NaHCO₃ (20 mL) and extracted with EtOAc (3×50 mL). The combined organic component was washed with brine (˜60 mL), dried (Na₂SO₄), filtered and concentrated. The crude material was then purified using a Biotage Horizon (24 g SiO₂, 0-50% EtOAc/hexanes) to yield Intermediate 19 (581 mg) as a white solid. LC-MS retention time=1.23 min; m/z=448.0 [M+H]⁺. (Column: Waters Aquity BEH C18, 2.0×50 mm, 1.7-μm particles. Solvent A=100% Water: 0.05% TFA. Solvent B=100% Acetonitrile: 0.05% TFA. Flow Rate=0.8 mL/min. Start % B=2. Final % B=98. Gradient Time=1.5 min. Wavelength=220 nm). ¹H NMR (400 MHZ, CDCl₃) δ ppm 9.10 (s, 1H), 7.98 (d, J=8.3 Hz, 1H), 7.68 (br. s., 1H), 7.05 (br. s., 1H), 6.68 (t, J=8.9 Hz, 1H), 6.44 (d, J=6.3 Hz, 2H), 5.25 (d, J=9.0 Hz, 1H), 4.54 (q, J=7.3 Hz, 1H), 2.94-2.86 (m, 1H), 2.81 (s, 3H), 2.72 (dd, J=13.1, 6.5 Hz, 1H), 1.39 (s, 9H).

### Intermediate 20

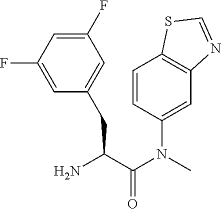 [SMILES: CN(C(=O)[C@@H](N)Cc1cc(F)cc(F)c1)c1ccc2scnc2c1]

TFA (1.0 mL, 13 mmol) was added to a stirred solution of Intermediate 19 (0.58 g, 1.2 mmol) in DCM (2 mL) and the reaction mixture was stirred at rt for 16 h. The crude reaction mixture was concentrated and the residue was dissolved in MeOH/DCM and 4M HCl in 1,4-dioxane (2 mL) and reconcentrated. The residue was redissolved in EtOH/toluene and then reconcentrated (3×) to yield an HCl salt of Intermediate 20 (0.55 g) as a white solid. LC-MS retention time=0.83 min; m/z=348.1 [M+H]⁺. (Column: Waters Aquity BEH C18, 2.0×50 mm, 1.7-μm particles. Solvent A=100% Water: 0.05% TFA. Solvent B=100% Acetonitrile: 0.05% TFA. Flow Rate=0.8 mL/min. Start % B=2. Final % B=98. Gradient Time=1.5 min. Wavelength=220 nm).

### Intermediate 25

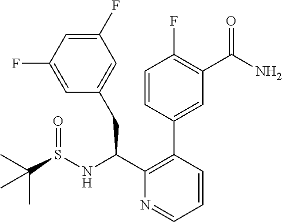 [SMILES: CC(C)(C)[S@@](=O)N[C@@H](Cc1cc(F)cc(F)c1)c1ncccc1-c1ccc(F)c(C(N)=O)c1]

Nitrogen was bubbled through a reaction mixture of Intermediate 15 (143 mg, 0.343 mmol), (3-carbamoyl-4-fluorophenyl)boronic acid (69.0 mg, 0.377 mmol) and potassium carbonate (104 mg, 0.754 mmol) in DME (1.5 mL) and water (0.5 mL) for 5 min. Then Pd(Ph₃P)₄ (39.6 mg, 0.034 mmol) was added, the reaction vessel was sealed and the reaction mixture was heated at 120° C. with microwave irradiation for 40 min. The reaction mixture was concentrated and the crude residue was purified using a Biotage Horizon (12 g SiO₂, 30-100% EtOAc/hexanes) to yield the title compound (63 mg). LC-MS retention time=1.40 min; m/z=476.4 [M+H]⁺. (Column: Phenomenex Luna C18 30×2.0 mm 3 μm. Solvent A=90% Water: 10% Acetonitrile: 0.1% TFA. Solvent B=10% Water: 90% Acetonitrile: 0.1% TFA. Flow Rate=1 mL/min. Start % B=0. Final % B=100. Gradient Time=2 minutes, then a 1-minute hold at 100% B. Wavelength=220 nm). ¹H NMR (400 MHZ, CDCl₃) δ ppm 8.70 (dd, J=4.8, 1.5 Hz, 1H), 7.62 (d, J=6.5 Hz, 1H), 7.42 (dd, J=7.8, 1.5 Hz, 1H), 7.30-7.25 (m, 1H), 7.16 (dd, J=11.3, 8.5 Hz, 1H), 7.03 (br s, 1H), 6.93 (br. s., 1H), 6.63-6.54 (m, 1H), 6.14 (d, J=6.3 Hz, 2H), 5.81 (br. s., 1H), 4.70 (td, J=9.2, 5.1 Hz, 1H), 4.47 (d, J=8.8 Hz, 1H), 3.26-3.12 (m, 2H), 1.21 (s, 9H).

### Intermediate 26

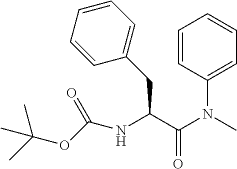 [SMILES: CN(C(=O)[C@H](Cc1ccccc1)NC(=O)OC(C)(C)C)c1ccccc1]

HATU (150 mg, 0.40 mmol) was added to a stirred solution of (S)-2-((tert-butoxycarbonyl)amino)-3-phenylpropanoic acid (100 mg, 0.38 mmol) and N-methylaniline (40 mg, 0.38 mmol) in DMF (2 mL) and DIPEA (0.13 mL, 0.75 mmol) and the reaction mixture was stirred at rt overnight. The reaction mixture diluted with EtOAc (˜8 mL) and washed with water (2×6 mL) and brine (˜6 mL) and the organic component was concentrated. The crude oil was then purified using a Biotage Horizon (4 g SiO₂, 10-20% EtOAc/hexanes) to yield Intermediate 26 (77 mg). LC-MS retention time=1.29 min; m/z=355.3 [M+H]⁺. (Column: Waters Aquity BEH C18 2.1×50 mm 1.7 μm. Solvent A=90% Water: 10% Acetonitrile: 0.05% TFA. Solvent B=10% Water: 90% Acetonitrile: 0.05% TFA. Flow Rate=0.8 mL/min. Start % B=2. Final % B=98. Gradient Time=1.5 minutes, then a 0.5-minute hold at 98% B. Wavelength=220 nm).

### Intermediate 27

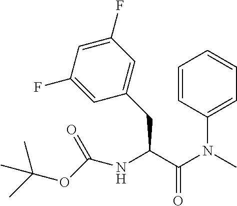 [SMILES: CN(C(=O)[C@H](Cc1cc(F)cc(F)c1)NC(=O)OC(C)(C)C)c1ccccc1]

HATU (150 mg, 0.40 mmol) was added to a stirred solution of (S)-2-((tert-butoxycarbonyl)amino)-3-(3,5-difluorophenyl)propanoic acid (114 mg, 0.377 mmol) and N-methylaniline (40 mg, 0.38 mmol) in DMF (2 mL) and DIPEA (0.13 mL, 0.75 mmol) and the reaction mixture was stirred at rt overnight. The reaction mixture diluted with EtOAc (˜8 mL) and washed with water (2×6 mL) and brine (˜6 mL) and the organic component was concentrated. The crude oil was then purified using a Biotage Horizon (4 g SiO₂, 10-20% EtOAc/hexanes) to yield Intermediate 27 (66 mg). LC-MS retention time=1.32 min; m/z=391.2 [M+H]⁺. (Column: Waters Aquity BEH C18 2.1×50 mm 1.7 μm. Solvent A=90% Water: 10% Acetonitrile: 0.05% TFA. Solvent B=10% Water: 90% Acetonitrile: 0.05% TFA. Flow Rate=0.8 mL/min. Start % B=2. Final % B=98. Gradient Time=1.5 minutes, then a 0.5-minute hold at 98% B. Wavelength=220 nm).

### Intermediate 28

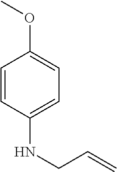

3-Bromoprop-1-ene (0.687 mL, 8.12 mmol) was added dropwise to a stirred solution of 4-methoxyaniline (1.0 g, 8.1 mmol), potassium carbonate (2.81 g, 20.3 mmol) and DMF (17 mL) and the reaction mixture was flushed with nitrogen, sealed and heated at 80° C. overnight. The reaction mixture was diluted with water (˜70 mL) and extracted with EtOAc (2×60 mL). The combined organic component was washed with brine (60 mL), dried (MgSO₄), filtered and concentrated. The crude oil was then purified using a Biotage Horizon (40 g SiO₂, 5-20% EtOAc/hexanes) to yield the title compound (652 mg). ¹H NMR (400 MHZ, CDCl₃) δ ppm 6.82-6.78 (m, 2H), 6.65-6.59 (m, 2H), 5.98 (ddt, J=17.3, 10.4, 5.3 Hz, 1H), 5.29 (app dq, J=17.2, 1.6 Hz, 1H), 5.17 (app dq, J=10.3, 1.4 Hz, 1H), 3.76 (s, 3H), 3.76-3.73 (m, 2H).

### Intermediate 29

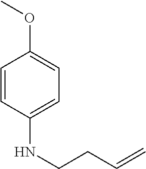

4-Bromobut-1-ene (1.10 g, 8.12 mmol) was added dropwise to a stirred solution of 4-methoxyaniline (1.0 g, 8.1 mmol), potassium carbonate (2.81 g, 20.3 mmol) and DMF (17 mL) and the reaction mixture was flushed with nitrogen, sealed and heated at 80° C. overnight. The reaction mixture was diluted with water (˜70 mL) and extracted with EtOAc (2×60 mL). The combined organic component was washed with brine (60 mL), dried (MgSO₄), filtered and concentrated. The crude oil was then purified using a Biotage Horizon (40 g SiO₂, 5-20% EtOAc/hexanes) to give the title compound (709 mg). ¹H NMR (400 MHZ, CDCl₃) δ ppm 6.83-6.77 (m, 2H), 6.64-6.58 (m, 2H), 5.84 (ddt, J=17.1, 10.2, 6.8 Hz, 1H), 5.19-5.10 (m, 2H), 3.77 (s, 3H), 3.16 (t, J=6.8 Hz, 2H), 2.39 (q, J=6.7 Hz, 2H).

### Intermediate 30

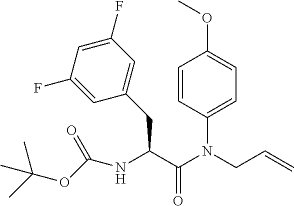 [SMILES: C=CCN(C(=O)[C@H](Cc1cc(F)cc(F)c1)NC(=O)OC(C)(C)C)c1ccc(OC)cc1]

HATU (713 mg, 1.88 mmol) was added to a stirred solution of Intermediate 28 (300 mg, 1.84 mmol) and (S)-2-((tert-butoxycarbonyl)amino)-3-(3,5-difluorophenyl)propanoic acid (554 mg, 1.838 mmol) in DMF (10 mL) and DIPEA (0.64 mL, 3.7 mmol) and the reaction mixture was allowed to stir at rt overnight. The reaction was diluted with water (˜50 mL) and extracted with EtOAc (2×50 mL). The combined organic components were washed with brine (30 mL), dried (MgSO₄), filtered and concentrated. The crude oil was then purified using a Biotage Horizon (40 g SiO₂, 10-25% EtOAc/hexanes) to yield the title compound (875 mg). ¹H NMR (400 MHZ, CDCl₃) δ ppm 6.89 (d, J=6.5 Hz, 3H), 6.71-6.63 (m, 1H), 6.47 (d, J=6.3 Hz, 2H), 5.80 (ddt, J=16.9, 10.3, 6.3 Hz, 1H), 5.22 (d, J=8.8 Hz, 1H), 5.14 (d, J=9.3 Hz, 1H), 5.06 (dd, J=17.1, 1.3 Hz, 1H), 4.52-4.42 (m, 1H), 4.28-4.15 (m, 2H), 3.84 (s, 3H), 2.88 (dd, J=13.3, 6.8 Hz, 1H), 2.67 (dd, J=13.2, 6.9 Hz, 1H), 1.39 (s, 9H).

### Intermediate 31

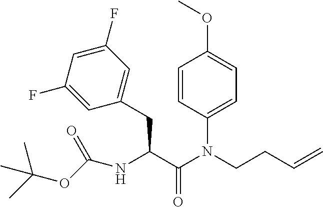 [SMILES: C=CCCN(C(=O)[C@H](Cc1cc(F)cc(F)c1)NC(=O)OC(C)(C)C)c1ccc(OC)cc1]

HATU (713 mg, 1.88 mmol) was added to a stirred solution of Intermediate 29 (326 mg, 1.84 mmol) and (S)-2-((tert-butoxycarbonyl)amino)-3-(3,5-difluorophenyl)propanoic acid (554 mg, 1.838 mmol) in DMF (10 mL) and DIPEA (0.64 mL, 3.7 mmol) and the reaction mixture was allowed to stir at rt overnight. The reaction was diluted with water (˜50 mL) and extracted with EtOAc (2×50 mL). The combined organic components were washed with brine (30 mL), dried (MgSO₄), filtered and concentrated. The crude oil was then purified using a Biotage Horizon (40 g SiO₂, 10-25% EtOAc/hexanes) to yield the title compound (908 mg). LC-MS retention time=2.28 min; m/z=483.5 [M+Na]⁺. (Column: Phenomenex Luna C18 30×2.0 mm 3 μm. Solvent A=90% Water: 10% Acetonitrile: 0.1% TFA. Solvent B=10% Water: 90% Acetonitrile: 0.1% TFA. Flow Rate=1 mL/min. Start % B=0. Final % B=100. Gradient Time=2 minutes, then a 1-minute hold at 100% B. Wavelength=220 nm). ¹H NMR (400 MHZ, CDCl₃) δ ppm 6.91 (d, J=6.0 Hz, 3H), 6.66 (tt, J=9.0, 2.3 Hz, 1H), 6.46 (d, J=6.0 Hz, 2H), 5.73 (ddt, J=17.1, 10.3, 6.7 Hz, 1H), 5.20 (d, J=8.5 Hz, 1H), 5.10-5.00 (m, 2H), 4.49-4.37 (m, 1H), 3.90-3.78 (m, 4H), 3.57 (dt, J=13.7, 7.0 Hz, 1H), 2.86 (dd, J=13.3, 6.8 Hz, 1H), 2.65 (dd, J=13.2, 6.9 Hz, 1H), 2.24 (q, J=6.5 Hz, 2H), 1.39 (s, 8H).

### Intermediate 32

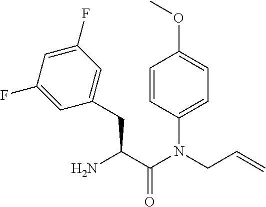 [SMILES: C=CCN(C(=O)[C@@H](N)Cc1cc(F)cc(F)c1)c1ccc(OC)cc1]

A solution of 4M HCl (2.3 mL, 9.3 mmol) in 1,4-dioxane was added to a stirred solution of Intermediate 30 (850 mg, 1.58 mmol) in THF (3 mL) and the reaction mixture was stirred at rt overnight. The reaction mixture was concentrated and then resubmitted to the reaction conditions detailed above and stirred at rt for 2d. The reaction mixture was then concentrated under high vacuum to yield an HCl salt of the title compound (677 mg) as an off-white solid. LC-MS retention time=1.24 min; m/z=347.4 [M+H]⁺. (Column: Phenomenex Luna C18 30×2.0 mm 3 μm. Solvent A=90% Water: 10% Acetonitrile: 0.1% TFA. Solvent B=10% Water: 90% Acetonitrile: 0.1% TFA. Flow Rate=1 mL/min. Start % B=0. Final % B=100. Gradient Time=2 minutes, then a 1-minute hold at 100% B. Wavelength=220 nm). ¹H NMR (400 MHZ, methanol-d₄) δ ppm 7.12-6.82 (m, 5H), 6.54 (d, J=6.0 Hz, 2H), 5.84 (ddt, J=16.9, 10.4, 6.4 Hz, 1H), 5.20-5.07 (m, 2H), 4.38-4.29 (m, 1H), 4.28-4.19 (m, 1H), 4.06 (t, J=7.0 Hz, 1H), 3.85 (s, 3H), 3.12 (dd, J=13.8, 6.8 Hz, 1H), 2.93 (dd, J=13.8, 7.3 Hz, 1H).

### Intermediate 33

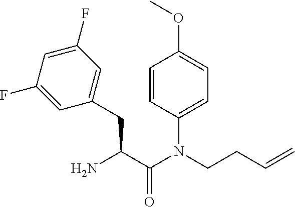 [SMILES: C=CCCN(C(=O)[C@@H](N)Cc1cc(F)cc(F)c1)c1ccc(OC)cc1]

A solution of 4M HCl (2.3 mL, 9.3 mmol) in 1,4-dioxane was added to a stirred solution of Intermediate 31 (870 mg, 1.549 mmol) in THF (3 mL) and the reaction mixture was stirred at rt overnight. Additional 4M HCl (˜0.5 mL) in 1,4-dioxane was added and the reaction mixture was stirred at rt for 2 d. The reaction mixture was then concentrated under high vacuum to yield an HCl salt of the title compound (689 mg) as an off-white solid. LC-MS retention time=1.32 min; m/z=361.4 [M+H]⁺. (Column: Phenomenex Luna C18 30×2.0 mm 3 μm. Solvent A=90% Water: 10% Acetonitrile: 0.1% TFA. Solvent B=10% Water: 90% Acetonitrile: 0.1% TFA. Flow Rate=1 mL/min. Start % B=0. Final % B=100. Gradient Time=2 minutes, then a 1-minute hold at 100% B. Wavelength=220 nm). ¹H NMR (400 MHZ, methanol-d₄) δ ppm 7.21-6.83 (m, 5H), 6.54 (d, J=5.0 Hz, 2H), 5.77 (d, J=7.3 Hz, 1H), 5.14-5.01 (m, 2H), 4.04 (br. s., 1H), 3.87 (br. s., 4H), 3.75-3.63 (m, 1H), 3.11 (dd, J=13.3, 6.0 Hz, 1H), 2.92 (dd, J=13.2, 7.2 Hz, 1H), 2.29 (d, J=5.5 Hz, 2H).

### Intermediate 34

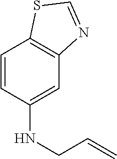

3-Bromoprop-1-ene (0.332 mL, 3.93 mmol) was added dropwise to a stirred mixture of benzo[d]thiazol-5-amine (590 mg, 3.93 mmol), potassium carbonate (1.36 g, 9.82 mmol) and DMF (10 mL) and then the reaction mixture was flushed with nitrogen, sealed and heated at 80° C. overnight. The reaction was diluted with water (˜70 mL), extracted with EtOAc (2×60 mL) and the combined organic component was washed with brine (60 mL), dried (MgSO₄), filtered and concentrated. The crude oil was purified using a Biotage Horizon (40 g SiO₂, 10-30%, then 100% EtOAc/hexanes) to yield the title compound (430 mg). ¹H NMR (400 MHZ, CDCl₃) δ ppm 8.91 (s, 1H), 7.69 (d, J=8.5 Hz, 1H), 7.33 (d, J=2.3 Hz, 1H), 6.84 (dd, J=8.8, 2.3 Hz, 1H), 6.01 (ddt, J=17.2, 10.4, 5.3 Hz, 1H), 5.35 (dd, J=17.1, 1.5 Hz, 1H), 5.22 (dd, J=10.3, 1.5 Hz, 1H), 4.02 (br. s., 1H), 3.88 (d, J=5.5 Hz, 2H).

### Intermediate 35

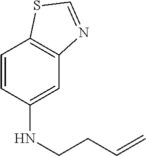

4-Bromobut-1-ene (530 mg, 3.93 mmol) was added dropwise to a stirred mixture of benzo[d]thiazol-5-amine (590 mg, 3.93 mmol), potassium carbonate (1.36 g, 9.82 mmol) and DMF (10 mL) and then the reaction mixture was flushed with nitrogen, sealed and heated at 80° C. overnight. The reaction was diluted with water (˜70 mL), extracted with EtOAc (2×60 mL) and the combined organic component was washed with brine (60 mL), dried (MgSO₄), filtered and concentrated. The crude oil was purified using a Biotage Horizon (40 g SiO₂, 10-30%, then 100% EtOAc/hexanes) to yield the title compound

(355 mg). ¹H NMR (400 MHZ, CDCl₃) δ ppm 8.92 (s, 1H), 7.69 (d, J=8.5 Hz, 1H), 7.32 (d, J=2.0 Hz, 1H), 6.81 (dd, J=8.5, 2.3 Hz, 1H), 5.87 (ddt, J=17.1, 10.2, 6.8 Hz, 1H), 5.23-5.11 (m, 2H), 3.89 (br. s., 1H), 3.29 (t, J=6.5 Hz, 2H), 2.46 (q, J=6.6 Hz, 2H).

### Intermediate 36

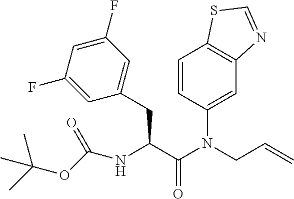 [SMILES: C=CCN(C(=O)[C@H](Cc1cc(F)cc(F)c1)NC(=O)OC(C)(C)C)c1ccc2scnc2c1]

HATU (892 mg, 2.35 mmol) was added to a stirred solution of Intermediate 34 (425 mg, 2.23 mmol) and (S)-2-((tert-butoxycarbonyl)amino)-3-(3,5-difluorophenyl)propanoic acid (673 mg, 2.23 mmol) in DMF (10 mL) and DIPEA (0.98 mL, 5.6 mmol) and the reaction mixture was stirred at rt overnight. The reaction mixture was diluted with water (˜30 mL), extracted with EtOAc (2×30 mL) and the combined organic components were washed with brine (˜30 mL), dried (MgSO₄), filtered and concentrated. The crude amber oil was purified using a Biotage Horizon (40 g SiO₂, 10-25% EtOAc/hexanes) to yield the title compound (652 mg). LC-MS retention time=1.86 min; m/z=496.4 [M+Na]⁺. (Column: Phenomenex Luna C18 30×2.0 mm 3 μm. Solvent A=90% Water: 10% Acetonitrile: 0.1% TFA. Solvent B=10% Water: 90% Acetonitrile: 0.1% TFA. Flow Rate=1 mL/min. Start % B=0. Final % B=100. Gradient Time=2 minutes, then a 1-minute hold at 100% B. Wavelength=220 nm). ¹H NMR (400 MHZ, CDCl₃) δ ppm 9.10 (s, 1H), 7.97 (d, J=8.0 Hz, 1H), 7.64 (br. s., 1H), 7.01 (br. s., 1H), 6.69 (t, J=8.9 Hz, 1H), 6.45 (d, J=5.3 Hz, 2H), 5.84 (ddt, J=16.9, 10.3, 6.5 Hz, 1H), 5.23 (d, J=8.8 Hz, 1H), 5.16 (d, J=10.0 Hz, 1H), 5.07 (dd, J=17.3, 1.3 Hz, 1H), 4.46 (q, J=7.4 Hz, 1H), 4.32 (d, J=6.3 Hz, 2H), 2.92 (dd, J=13.3, 7.5 Hz, 1H), 2.73 (dd, J=13.1, 6.3 Hz, 1H), 1.39 (s, 9H).

### Intermediate 37

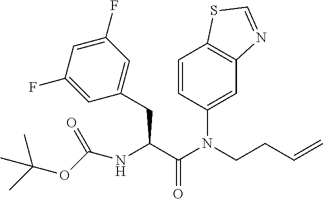 [SMILES: C=CCCN(C(=O)[C@H](Cc1cc(F)cc(F)c1)NC(=O)OC(C)(C)C)c1ccc2scnc2c1]

HATU (684 mg, 1.80 mmol) was added to a stirred solution of Intermediate 35 (350 mg, 1.71 mmol) and (S)-2-((tert-butoxycarbonyl)amino)-3-(3,5-difluorophenyl)propanoic acid (516 mg, 1.713 mmol) in DMF (10 mL) and DIPEA (0.75 mL, 4.3 mmol) and the reaction mixture was stirred at rt overnight. The reaction mixture was diluted with water (˜30 mL), extracted with EtOAc (2×30 mL) and the combined organic components were washed with brine (˜30 mL), dried (MgSO₄), filtered and concentrated. The crude amber oil was purified using a Biotage Horizon (40 g SiO₂, 10-25% EtOAc/hexanes) to yield the title compound (406 mg). LC-MS retention time=1.94 min; m/z=388.4 [M+H-Boc]⁺. (Column: Phenomenex Luna C18 30×2.0 mm 3 μm. Solvent A=90% Water: 10% Acetonitrile: 0.1% TFA. Solvent B=10% Water: 90% Acetonitrile: 0.1% TFA. Flow Rate=1 mL/min. Start % B=0. Final % B=100. Gradient Time=2 minutes, then a 1-minute hold at 100% B. Wavelength=220 nm). ¹H NMR (400 MHZ, CDCl₃) δ ppm 9.11 (s, 1H), 7.99 (d, J=7.8 Hz, 1H), 7.84-7.43 (m, 1H), 6.69 (t, J=9.0 Hz, 1H), 6.46 (br. s., 2H), 5.74 (ddt, J=17.0, 10.4, 6.7 Hz, 1H), 5.21 (d, J=8.5 Hz, 1H), 5.12-5.02 (m, 2H), 4.46-4.37 (m, 1H), 3.98-3.86 (m, 1H), 3.80-3.66 (m, 1H), 2.91 (dd, J=13.2, 7.4 Hz, 1H), 2.71 (dd, J=13.1, 6.3 Hz, 1H), 2.29 (q, J=6.8 Hz, 2H), 1.47-1.33 (m, 9H).

### Intermediate 38

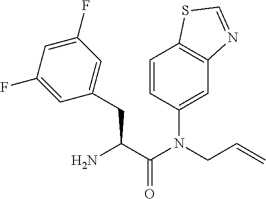 [SMILES: C=CCN(C(=O)[C@@H](N)Cc1cc(F)cc(F)c1)c1ccc2scnc2c1]

A solution of 4M HCl (2.4 mL, 9.5 mmol) in 1,4-dioxane was added to a stirred solution of Intermediate 36 (450 mg, 0.950 mmol) in THF (4 mL) and the reaction mixture was stirred at rt overnight. The reaction mixture was concentrated, treated with MeOH (˜5 mL) and reconcentrated (2×) to yield an HCl salt of the title compound (455 mg) as a violet solidified foam which was used without further purification. LC-MS retention time=1.23 min; m/z=374.3 [M+H]⁺. (Column: Phenomenex Luna C18 30×2.0 mm 3 μm. Solvent A=90% Water: 10% Acetonitrile: 0.1% TFA. Solvent B=10% Water: 90% Acetonitrile: 0.1% TFA. Flow Rate=1 mL/min. Start % B=0. Final % B=100. Gradient Time=2 minutes, then a 1-minute hold at 100% B. Wavelength=220 nm).

### Intermediate 39

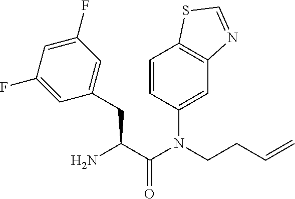 [SMILES: C=CCCN(C(=O)[C@@H](N)Cc1cc(F)cc(F)c1)c1ccc2scnc2c1]

A solution of 4M HCl (2.4 mL, 9.8 mmol) in 1,4-dioxane was added to a stirred solution of Intermediate 37 (396 mg, 0.812 mmol) in THF (4 mL) and the reaction mixture was stirred at rt overnight. The reaction mixture was concentrated, treated with MeOH (˜5 mL) and reconcentrated (2×) to yield an HCl salt of the title compound (455 mg) as a pink/orange solidified foam which was used without further purification. LC-MS retention time=1.30 min; m/z=388.4 [M+H]⁺. (Column: Phenomenex Luna C18 30×2.0 mm 3 μm. Solvent A=90% Water: 10% Acetonitrile: 0.1% TFA. Solvent B=10% Water: 90% Acetonitrile: 0.1% TFA. Flow Rate=1 mL/min. Start % B=0. Final % B=100. Gradient Time=2 minutes, then a 1-minute hold at 100% B. Wavelength=220 nm).

### Intermediate 51

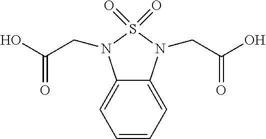 [SMILES: O=C(O)CN1c2ccccc2N(CC(=O)O)S1(=O)=O]

Step 1.

A mixture of 1,3-dihydrobenzo[c][1,2,5]thiadiazole 2,2-dioxide (120 mg, 0.705 mmol), cesium carbonate (505 mg, 1.55 mmol) and tert-butyl 2-bromoacetate (0.22 mL, 1.5 mmol) in DMF (5 mL) was sealed and heated at 70° C. for 8 h . The reaction mixture was poured into water and extracted with EtOAc. The organic component was washed with 5% citric acid and brine, dried over MgSO₄, filtered and concentrated. The residual gum was purified by FCC (40 g silica gel, eluted with gradient 10%-50% EtOAc-Hexanes) to afford di-tert-butyl 2,2′-(2,2-dioxidobenzo[c][1,2,5]thiadiazole-1,3-diyl)diacetate (251 mg) as an off-white solid. ¹H NMR (400 MHZ, CDCl₃) δ ppm 7.11-6.96 (m, 2H), 6.80-6.60 (m, 2H), 4.35 (s, 4H), 1.47 (s, 18H).

Step 2.

Di-tert-butyl 2,2′-(2,2-dioxidobenzo[c][1,2,5]thiadiazole-1,3-diyl)diacetate (251 mg, 0.630 mmol) was stirred in 4M hydrogen chloride in 1,4-dioxane (3.15 mL, 12.6 mmol) at rt overnight. The solvent was removed and the residual off-white solid was triturated with 4:1 hexanes-EtOAc, filtered and dried in vacuo to afford the title compound Intermediate 51 (177 mg) as an off-white powder. LC-MS retention time=0.20 min; m/z=285.2 [M−H]⁻. (Column: Phenomenex Luna C18 2.0×30 mm 3 μm; Solvent A=95% Water: 5% Acetonitrilel 10 mM Ammonium Acetate; Solvent B=5% Water: 95% Acetonitrile 10 mM Ammonium Acetate; Flow Rate=1.0 mL/min. Start % B=0; Final % B=100; Gradient Time=2.0 minutes; Wavelength=220 nm). ¹H NMR (400 MHZ, methanol-d₄) δ ppm 7.03 (dd, J=5.6, 3.1 Hz, 2H), 6.89 (dd, J=5.5, 3.3 Hz, 2H), 4.55 (s, 4H).

### Intermediate 52

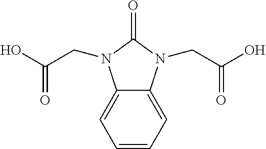 [SMILES: O=C(O)Cn1c(=O)n(CC(=O)O)c2ccccc21]

Step 1.

A mixture of 1H-benzo[d]imidazol-2(3H)-one (150 mg, 1.12 mmol), cesium carbonate (802 mg, 2.46 mmol) and tert-butyl 2-bromoacetate (0.35 mL, 2.35 mmol) in acetone (10 mL) was sealed and heated in an oil bath at 65° C. for 6 h. The reaction mixture was filtered and concentrated in vacuo. The residual solid was taken up into DCM (5 mL) and purified by FCC (80 g silica gel, eluted with gradient 10%-60% EtOAc-hexanes) to afford di-tert-butyl 2,2′-(2-oxo-1H-benzo[d]imidazole-1,3(2H)-diyl)diacetate (328 mg) as a white solid. LC-MS retention time=0.74 min; m/z=363.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm). ¹H NMR (400 MHZ, CDCl₃) δ ppm 7.19-7.06 (m, 2H), 6.91 (dd, J=5.8, 3.3 Hz, 2H), 4.56 (s, 4H), 1.48 (s, 18H).

Step 2.

Di-tert-butyl 2,2′-(2-oxo-1H-benzo[d]imidazole-1,3(2H)-diyl)diacetate (320 mg, 0.883 mmol) was stirred in 4M hydrogen chloride in 1,4-dioxane (3.97 mL, 15.9 mmol) at rt overnight. The solvent was removed and the residual solid was triturated with ether, filtered and dried in vacuo to afford the title compound Intermediate 52 (215 mg) as a white solid. LC-MS retention time=0.82 min; m/z=250.9 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Intermediate 53

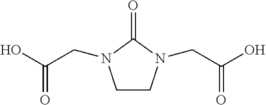 [SMILES: O=C(O)CN1CCN(CC(=O)O)C1=O]

Step 1.

A mixture of 1H-imidazol-2(3H)-one (100 mg, 1.19 mmol), cesium carbonate (853 mg, 2.62 mmol) and benzyl 2-bromoacetate (0.40 mL, 2.50 mmol) in acetone (10 mL) was sealed and heated in an oil bath at 65° C. for 6 h. The reaction mixture was filtered and concentrated in vacuo. The residual solid was purified by FCC (80 g silica gel, eluted with gradient 30%˜100% EtOAc-hexanes) to afford dibenzyl 2,2′-(2-oxo-1H-imidazole-1,3(2H)-diyl)diacetate (240 mg) as a colorless oil. LC-MS retention time=1.16 min; m/z=381.3 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm). ¹H NMR (400 MHZ, CDCl₃) δ ppm 7.46-7.32 (m, 10H), 6.33 (s, 2H), 5.22 (s, 4H), 4.48 (s, 4H).

Step 2.

10% Pd/C (24.3 mg, 0.023 mmol) was added to a solution of dibenzyl 2,2′-(2-oxo-1H-imidazole-1,3(2H)-diyl)diacetate (87 mg, 0.23 mmol) in MeOH (4 mL) and after purging the reaction mixture with N₂ (2×), it was placed under 1 atm of H₂ (balloon) and stirred at rt for 2 h. The reaction mixture was filtered through a PVDF syringe filter, concentrated to yield the title compound which was used without further purification. LC-MS retention time=0.60 min; m/z=203.0 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm). ¹H NMR (400 MHZ, methanol-d₄) δ ppm 3.97 (s, 4H), 3.56 (s, 4H).

### Intermediate 54

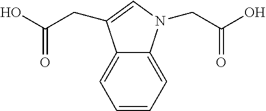 [SMILES: O=C(O)Cc1cn(CC(=O)O)c2ccccc12]

Step 1.

A mixture of ethyl 2-(1H-indol-3-yl)acetate (500 mg, 2.46 mmol), cesium carbonate (1.04 g, 3.20 mmol) and ethyl 2-bromoacetate (0.33 mL, 3.0 mmol) in acetone (20 mL) was sealed and heated in an oil bath at 65° C. for 6 h. The reaction mixture was filtered and the filtrate was concentrated in vacuo. The residual oil was taken up into DMF and then purified by preparative HPLC to afford the title compound diethyl 2,2′-(1H-indole-1,3-diyl)diacetate (255 mg) as an off-white solid. LC-MS retention time=1.23 min; m/z=290.3 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm). ¹H NMR (400 MHZ, CDCl₃) δ ppm 7.65 (d, J=8.0 Hz, 1H), 7.32-7.22 (m, 3H), 7.21-7.11 (m, 2H), 4.83 (s, 2H), 4.21 (dq, J=19.0, 7.1 Hz, 4H), 3.80 (d, J=0.8 Hz, 2H), 1.29 (t, J=7.2 Hz, 6H).

Step 2.

To a solution of diethyl 2,2′-(1H-indole-1,3-diyl)diacetate (250 mg, 0.864 mmol) in MeOH (4 mL) and THF (4 mL) was added a premade solution of lithium hydroxide monohydrate (181 mg, 4.32 mmol) in water (4 mL). The reaction mixture was stirred at rt overnight and then concentrated. The residual liquid was acidified to pH 2 using 1 M HCl. The resulting solid was collected by filtration, washed with water and dried in vacuo to afford the title compound Intermediate 54 (189 mg) as a white solid. LC-MS retention time=0.77 min; m/z=234.0 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Intermediate 55

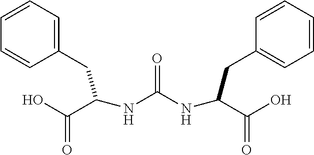 [SMILES: O=C(N[C@@H](Cc1ccccc1)C(=O)O)N[C@@H](Cc1ccccc1)C(=O)O]

Step 1.

To a mixture of an HCl salt of (S)-benzyl 2-amino-3-phenylpropanoatem (306 mg, 1.05 mmol) in acetonitrile (5 mL) at 0° C. was added triethylamine (0.42 mL, 3.0 mmol) and CDI (81.0 mg, 0.500 mmol). The reaction mixture was stirred at this temperature for 1 h and then sealed and heated in an oil bath at 55° C. for 2 h. The reaction mixture was cooled to rt, diluted with EtOAc (20 mL) and washed with water (40 mL). The organic component was washed with 5% citric acid and brine, dried over MgSO₄, filtered and concentrated. The residual solid was triturated with hexanes to afford the title compound (2S,2′S)-dibenzyl 2,2′-(carbonylbis(azanediyl))bis(3-phenylpropanoate) (205 mg) as a white solid. LC-MS retention time=1.45 min; m/z=357.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

Step 2.

10% Pd/C (35.3 mg, 0.033 mmol) was added to a solution of (2S,2′S)-dibenzyl 2,2′-(carbonylbis(azanediyl))bis(3-phenylpropanoate) (178 mg, 0.332 mmol) in EtOAc (20 mL) and after purging the sample with N₂ (2×) it was placed under 1 atm of H₂ (balloon) and stirred at rt for 2 h. The reaction mixture was filtered through a PVDF syringe filter and concentrated in vacuo to afford the title compound Intermediate 55 (115 mg) as a white solid. LC-MS retention time=0.92 min; m/z=357.0 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Intermediate 56

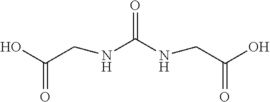 [SMILES: O=C(O)CNC(=O)NCC(=O)O]

Step 1.

To a mixture of benzyl 2-aminoacetate, HCl (212 mg, 1.05 mmol) in acetonitrile (5 mL) at 0° C. was added triethylamine (0.42 mL, 3.0 mmol) and CDI (81 mg, 0.50 mmol). The reaction mixture was stirred at this temperature for 1 h and then sealed and heated in an oil bath at 55° C. for 2 h. The reaction mixture was cooled to rt, diluted with EtOAc (20 mL) and washed with water (40 mL). The organic component was washed with 5% citric acid and brine, dried over MgSO₄, filtered and concentrated. The residual solid was triturated with hexanes to afford the title compound (120 mg) as a white solid. LC-MS retention time=1.08 min; m/z=357.1 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

Step 2.

10% Pd/C (35.8 mg, 0.034 mmol) was added to a solution of dibenzyl 2,2′-(carbonylbis(azanediyl))diacetate (120 mg, 0.337 mmol) in EtOAc (10 mL). After purging the sample with N₂ (2×) it was placed under 1 atm of H₂ (balloon) and stirred at rt for 2 h. The reaction mixture was filtered through a PVDF syringe filter and concentrated in vacuo to afford the title compound (41 mg) as a white solid. LC-MS retention time=0.77 min; m/z=177.0 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Intermediate 57

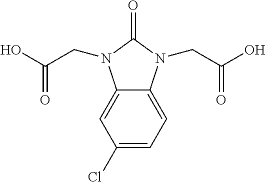 [SMILES: O=C(O)Cn1c(=O)n(CC(=O)O)c2cc(Cl)ccc21]

Step 1.

A mixture of 5-chloro-1H-benzo[d]imidazol-2(3H)-one (200 mg, 1.19 mmol), cesium carbonate (850 mg, 2.61 mmol) and tert-butyl 2-bromoacetate (0.37 mL, 2.5 mmol) in acetone (10 mL) was sealed and heated in an oil bath at 65° C. for 6 h. The reaction mixture was filtered and concentrated in vacuo, taken up into DCM (20 mL), washed with 5% citric acid and brine, dried over MgSO₄, filtered and concentrated in vacuo. The residual solid was recrystallized from 2:1 hexanes-EtOAc (10 mL) to afford di-tert-butyl 2,2′-(5-chloro2-oxo-1H-benzo[d]imidazole-1,3(2H)-diyl)diacetate (160 mg) as a white solid. LC-MS retention time=1.38 min; m/z=285.1 [M-2(t-Bu)+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

Step 2.

Di-tert-butyl 2,2′-(5-chloro-2-oxo-1H-benzo[d]imidazole-1,3(2H)-diyl)diacetate (160 mg, 0.403 mmol) was stirred in 4M hydrogen chloride in 1,4-dioxane (2.02 mL, 8.06 mmol) at rt overnight. The reaction mixture was concentrated and the residual solid was triturated with EtOAc, filtered and dried in vacuo to afford the title compound Intermediate 57 (71 mg) as a white solid. LC-MS retention time=0.84 min; m/z=284.9 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm). ¹H NMR (400 MHZ, methanol-d₄) δ ppm 7.22 (d, J=1.5 Hz, 1H), 7.18-7.06 (m, 2H), 4.71 (d, J=1.8 Hz, 4H).

### Intermediate 58

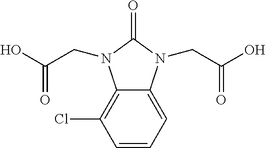 [SMILES: O=C(O)Cn1c(=O)n(CC(=O)O)c2c(Cl)cccc21]

Step 1.

A mixture of 4-chloro-1H-benzo[d]imidazol-2(3H)-one (200 mg, 1.19 mmol), cesium carbonate (850 mg, 2.61 mmol) and tert-butyl 2-bromoacetate (0.37 mL, 2.5 mmol) in acetone (10 mL) was sealed and heated in an oil bath at 65° C. for 6 h. The reaction mixture was filtered and concentrated in vacuo, and the residual oil was taken up into DCM (5 mL) and purified by FCC (80 g silica gel, eluted with gradient 10˜40% EtOAc-Hexanes) to afford di-tert-butyl 2,2′-(4-chloro2-oxo-1H-benzo[d]imidazole-1,3(2H)-diyl)diacetate (320 mg) as a white foam. LC-MS retention time=1.41 min; m/z=285.1 [M-2(t-Bu)+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

Step 2.

Di-tert-butyl 2,2′-(4-chloro-2-oxo-1H-benzo[d]imidazole-1,3(2H)-diyl)diacetate (320 mg, 0.806 mmol) was stirred in 4 M hydrogen chloride in 1,4-dioxane (4.03 mL, 16.1 mmol) at rt overnight. The reaction mixture was concentrated and the residual solid was triturated with ether, filtered and dried in vacuo to afford the title compound Intermediate 58 (220 mg) as a white solid. LC-MS retention time=0.81 min; m/z=284.9 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Intermediate 59

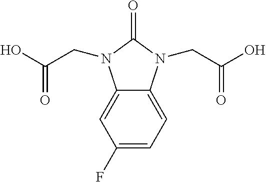 [SMILES: O=C(O)Cn1c(=O)n(CC(=O)O)c2cc(F)ccc21]

Step 1.

A mixture of 5-fluoro-1H-benzo[d]imidazol-2(3H)-one (200 mg, 1.26 mmol), cesium carbonate (905 mg, 2.78 mmol) and tert-butyl 2-bromoacetate (0.39 mL, 2.65 mmol) in acetone (10 mL) was sealed and heated in an oil bath at 65° C. for 6 h. The reaction mixture was filtered and concentrated in vacuo, and the residual oil taken up into DCM (5 mL) and purified by FCC (40 g silica gel, eluted with gradient 10˜50% EtOAc-Hexanes) to afford di-tert-butyl 2,2′-(5-fluoro2-oxo-1H-benzo[d]imidazole-1,3(2H)-diyl)diacetate (370 mg) as a white solid. LC-MS retention time=1.31 min; m/z=269.2 [M-2(t-Bu)+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

Step 2.

Di-tert-butyl 2,2′-(5-fluoro-2-oxo-1H-benzo [d] imidazole-1,3 (2H)-diyl)diacetate (370 mg, 0.973 mmol) was stirred in 4 M hydrogen chloride in 1,4-dioxane (4.86 mL, 19.4 mmol) at rt overnight. The reaction mixture was concentrated and the residual solid was triturated with EtOAc, filtered and dried in vacuo to afford the title compound Intermediate 59 (230 mg) as a white solid. LC-MS retention time=0.78 min; m/z=268.9 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Intermediate 60

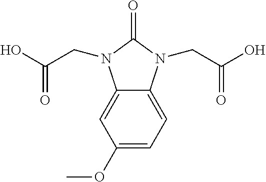 [SMILES: COc1ccc2c(c1)n(CC(=O)O)c(=O)n2CC(=O)O]

Step 1.

A mixture of 5-methoxy-1H-benzo[d]imidazol-2(3H)-one (200 mg, 1.22 mmol), cesium carbonate (873 mg, 2.68 mmol) and tert-butyl 2-bromoacetate (0.38 mL, 2.56 mmol) in acetone (10 mL) was sealed and heated in an oil bath at 65° C. for 6 h. The reaction mixture was filtered and concentrated in vacuo, taken up into DCM (20 mL), washed it with 5% citric acid and brine, dried over MgSO₄, filtered and concentrated in vacuo. The residual solid was triturated with 4:1 hexanes-EtOAc (10 mL) and dried in vacuo to afford di-tert-butyl 2,2′-(5-fluoro2-oxo-1H-benzo[d]imidazole-1,3(2H)-diyl)diacetate (351 mg) as a white solid. LC-MS (Condition AW-1) retention time=1.28 min; m/z=281.1 [M-2(t-Bu)+H]⁺. (Column: Waters Aquity BEH C18 2.1×50 mm particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

Step 2.

Di-tert-butyl 2,2′-(5-methoxy-2-oxo-1H-benzo[d]imidazole-1,3(2H)-diyl)diacetate (350 mg, 0.892 mmol) was stirred in 4 M hydrogen chloride in 1,4-dioxane (4.46 mL, 17.8 mmol) at rt overnight. The reaction mixture was concentrated and the residual solid was triturated with ether, filtered and dried in vacuo to afford the title compound Intermediate 60 (220 mg) as a white solid. LC-MS retention time=0.78 min; m/z=280.9 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Intermediate 61

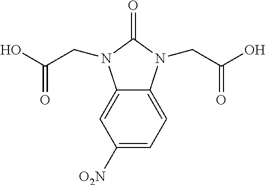 [SMILES: O=C(O)Cn1c(=O)n(CC(=O)O)c2cc([N+](=O)[O-])ccc21]

Step 1.

A mixture of 5-nitro-1H-benzo[d]imidazol-2(3H)-one (200 mg, 1.12 mmol), cesium carbonate (800 mg, 2.46 mmol) and tert-butyl 2-bromoacetate (0.35 mL, 2.3 mmol) in DMF (5 mL) was sealed and heated in an oil bath at 65° C. for 6 h. The reaction mixture was poured into warm water (50 mL), cooled to rt, filtered and dried in vacuo to afford di-tert-butyl 2,2′-(5-nitro-2-oxo-1H-benzo[d]imidazole-1,3(2H)-diyl)diacetate (340 mg) as a white solid. LC-MS retention time=1.31 min; m/z=295.9 [M-2(t-Bu)+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

Step 2.

Di-tert-butyl 2,2′-(5-nitro-2-oxo-1H-benzo[d]imidazole-1,3(2H)-diyl)diacetate (200 mg, 0.491 mmol) was stirred in 4 M hydrogen chloride in 1,4-dioxane (2.45 mL, 9.82 mmol) at rt overnight. The reaction mixture was concentrated and the residual solid was triturated with ether, filtered and dried in vacuo to afford the title compound Intermediate 61 (145 mg) as a white solid. LC-MS (Condition AW-1) retention time=0.76 min; m/z=295.9 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Intermediate 62

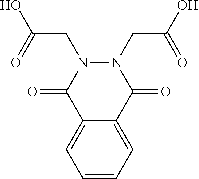 [SMILES: O=C(O)Cn1c(=O)c2ccccc2c(=O)n1CC(=O)O]

Step 1.

A mixture of 2,3-dihydrophthalazine-1,4-dione (200 mg, 1.23 mmol), cesium carbonate (884 mg, 2.71 mmol) and tert-butyl 2-bromoacetate (0.382 mL, 2.59 mmol) in DMF (10 mL) was sealed and heated in an oil bath at 80° C. for 6 h. The reaction mixture was poured into water (50 mL) and extracted with EtOAc. The organic component was washed with 5% citric acid and brine, dried over MgSO₄, filtered, and dried in vacuo. The residue was purified by FCC (40 g silica gel cartridge, eluted with gradient 10˜50% EtOAc-hexanes) to afford di-tert-butyl 2,2′-(1,4-dioxophthalazine-2,3(1H,4H)-diyl)diacetate (357 mg) as a colorless gum. LC-MS retention time=1.34 min; m/z=279.15 [M-2(t-Bu)+H]⁺. (Column: Waters Aquity BEH C18 2.1×50 mm particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm). ¹H NMR (400 MHZ, CDCl₃) δ ppm 8.53-8.36 (m, 1H), 8.17-8.04 (m, 1H), 7.82 (ddd, J=7.3, 5.1, 1.6 Hz, 2H), 4.79 (s, 2H), 4.71 (s, 2H), 1.50 (s, 9H), 1.55 (s, 9H).

Step 2.

Di-tert-butyl 2,2′-(1,4-dioxophthalazine-2,3(1H,4H)-diyl)diacetate (205 mg, 0.525 mmol) was stirred in 4 M hydrogen chloride in 1,4-dioxane (1.97 mL, 7.88 mmol) at rt overnight. The reaction mixture was concentrated and the residual solid was triturated with ether, filtered and dried in vacuo to afford the title compound Intermediate 62 (134 mg) as a white solid. LC-MS retention time=0.75 min; m/z=279.0 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm). ¹H NMR (400 MHZ, methanol-d₄) δ ppm 8.47-8.29 (m, 1H), 8.26-8.13 (m, 1H), 7.96 (dtd, J=19.8, 7.4, 1.3 Hz, 2H), 4.96 (s, 2H), 4.83 (s, 2H).

### Intermediate 63

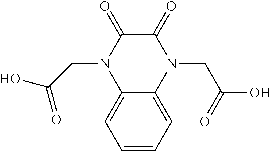 [SMILES: O=C(O)Cn1c(=O)c(=O)n(CC(=O)O)c2ccccc21]

Step 1.

A mixture of quinoxaline-2,3(1H,4H)-dione (200 mg, 1.23 mmol), cesium carbonate (884 mg, 2.71 mmol) and benzyl 2-bromoacetate (0.41 mL, 2.6 mmol) in DMF (8 mL) was sealed and heated in an oil bath at 80° C. for 6 h. The reaction mixture was poured it into water (50 mL) and extracted with EtOAc. The organic component was washed with 5% citric acid and brine, dried over MgSO₄, filtered and dried in vacuo. The residue was triturated with 4:1 hexanes-EtOAc to afford dibenzyl 2,2′-(2,3-dioxo-2,3-dihydroquinoxaline-1,4-diyl)diacetate (420 mg) as a light yellow solid. LC-MS retention time=1.23 min; m/z=459.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

Step 2.

To a solution of dibenzyl 2,2′-(2,3-dioxo-2,3-dihydroquinoxaline-1,4-diyl)diacetate (210 mg, 0.458 mmol) in THF (20 mL) was added 10% Pd/C (24 mg, 0.023 mmol). The reaction mixture was stirred under a H₂ balloon for 2 h, filtered and then concentrated. The residual solid was triturated with ether, filtered and dried in vacuo to afford the title compound Intermediate 63 (107 mg) as a white solid. LC-MS retention time=0.68 min; m/z=278.9 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm). ¹H NMR (400 MHZ, methanol-d₄) δ ppm 7.34 (s, 4H), 5.08 (s, 4H).

### Intermediate 64

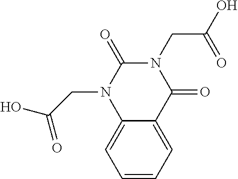 [SMILES: O=C(O)Cn1c(=O)c2ccccc2n(CC(=O)O)c1=O]

Step 1.

A mixture of quinazoline-2,4(1H,3H)-dione (200 mg, 1.23 mmol), cesium carbonate (884 mg, 2.71 mmol) and benzyl 2-bromoacetate (0.41 mL, 2.59 mmol) in DMF (8 mL) was sealed and heated in an oil bath at 80° C. for 6 h. The reaction mixture was poured into water (50 mL) and extracted with EtOAc. The organic component was washed with 5% citric acid and brine, dried over MgSO₄, filtered and dried in vacuo. The residue was purified by FCC (40 g silica gel cartridge, eluted with gradient 10˜50% EtOAc-hexanes) to afford dibenzyl 2,2′-(2,4-dioxoquinazoline-1,3(2H,4H)-diyl)diacetate (391 mg) as a colorless gum. LC-MS retention time=1.34 min; m/z=459.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm). ¹H NMR (400 MHZ, CDCl₃) δ ppm 8.27 (dd, J=8.0, 1.5 Hz, 1H), 7.64 (td, J=7.9, 1.5 Hz, 1H), 7.44-7.30 (m, 11H), 6.95 (d, J=8.3 Hz, 1H), 5.24 (s, 4H), 4.98 (s, 2H), 4.93 (s, 2H).

Step 2.

To a solution of dibenzyl 2,2′-(2,4-dioxoquinazoline-1,3(2H,4H)-diyl)diacetate (200 mg, 0.436 mmol) in ethyl acetate (5 mL) was added 10% Pd/C (23 mg, 0.022 mmol). The reaction mixture was stirred under a H₂ balloon for 2 h, filtered and concentrated. The residual solid was triturated with ether, filtered and dried in vacuo to afford the title compound Intermediate 64 (120 mg) as a white solid. LC-MS retention time=0.74 min; m/z=278.9 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Intermediate 65

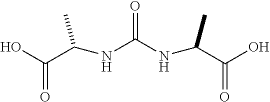

Step 1.

To a mixture of an HCl salt of (S)-benzyl 2-aminopropanoate(279 mg, 1.30 mmol) in acetonitrile (5 mL) at 0° C. was added triethylamine (0.52 mL, 3.70 mmol) and CDI (100 mg, 0.617 mmol). The formed yellow mixture was stirred at this temperature for 1 h, sealed and heated in an oil bath at 55° C. for 2 h. The reaction mixture was cooled to rt, diluted with EtOAc (20 mL) and extracted with water (40 mL). The organic component was washed with 5% citric acid and brine, dried over MgSO₄, filtered and concentrated. The residual solid was triturated with hexanes to afford (2S,2′S)-dibenzyl 2,2′-(carbonylbis(azanediyl))dipropanoate (120 mg) as a white solid. LC-MS retention time=1.16 min; m/z=385.3 [M+H]⁺. (Column: Waters Aquity BEH C18 2.1×50 mm 1.7 μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

Step 2.

10% Pd/C (33 mg, 0.031 mmol) was added to a solution of (2S,2′S)-dibenzyl 2,2′-(carbonylbis(azanediyl))dipropanoate (120 mg, 0.312 mmol) in EtOAc (10 mL). After purging the sample with N₂ (2×) it was placed under 1 atm of H₂ (balloon) and stirred at rt for 2 h. The reaction mixture was filtered through a PVDF syringe filter and concentrated in vacuo to afford the title compound Intermediate 65 (63 mg) as a white solid. LC-MS retention time=0.53 min; m/z=205.0 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Intermediate 66

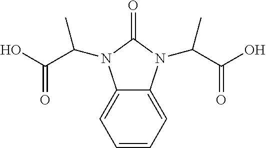 [SMILES: CC(C(=O)O)n1c(=O)n(C(C)C(=O)O)c2ccccc21]

Step 1.

To a solution of (S)-2-bromopropanoic acid (1.08 g, 7.06 mmol) and TEA (1.08 mL, 7.77 mmol) in DCM (20 mL) at 0° C. was added benzyl chloroformate (1.06 mL, 7.06 mmol) dropwise. After stirring at this temperature for 10 min, DMAP (0.086 g, 0.71 mmol) was added and the slurry was stirred for another 30 min and then at rt for 2 h. The reaction mixture was diluted with DCM (20 mL), washed with water, 1 M HCl (aq) and brine, dried it over MgSO₄, filtered and concentrated in vacuo. The residual oil was purified by FCC (80 g silica gel cartridge, eluted with gradient 0-30% EtOAc-Hexanes) to afford benzyl 2-bromopropanoate (1.21 g) as a colorless oil. Absolute stereochemistry not determined. ¹H NMR (400 MHZ, CDCl₃) δ ppm 7.54-7.32 (m, 5H), 5.23 (d, J=1.0 Hz, 2H), 4.44 (q, J=6.9 Hz, 1H), 1.87 (d, J=7.0 Hz, 3H).

Step 2.

A mixture of 1H-benzo[d]imidazol-2(3H)-one (84 mg, 0.63 mmol), cesium carbonate (449 mg, 1.38 mmol) and benzyl 2-bromopropanoate (320 mg, 1.32 mmol) in DMF (5 mL) was sealed and heated in a microwave system at 85° C. for 2 h. The reaction mixture was diluted with DCM (20 mL) and poured into water. The organic component was washed with brine, dried over MgSO₄, filtered and concentrated in vacuo. The residual oil was purified by FCC (40 g silica gel, eluted with gradient 15-60% EtOAc-hexanes) to afford dibenzyl 2,2′-(2-oxo-1H-benzo[d]imidazole-1,3(2H)-diyl)dipropanoate (248 mg) as a gum. LC-MS retention time=1.39 min; m/z=459.5 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

Step 3.

A mixture of dibenzyl 2,2′-(2-oxo-1H-benzo[d]imidazole-1,3(2H)-diyl)dipropanoate (248 mg, 0.54 mmol) and 20% Pd/C (28.8 mg, 0.054 mmol) in MeOH (5 mL) was placed under a H₂ balloon for 2 h. The reaction mixture was filtered and concentrated in vacuo to afford the title compound Intermediate 66 (150 mg) as a colorless gum. LC-MS retention time=0.82 min; m/z=278.9 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Intermediate 67

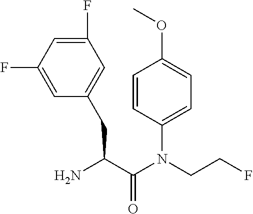 [SMILES: COc1ccc(N(CCF)C(=O)[C@@H](N)Cc2cc(F)cc(F)c2)cc1]

Step 1.

A mixture of 4-methoxyaniline (123 mg, 0.999 mmol), 1-fluoro-2-iodoethane (94 μl, 1.10 mmol) and sodium iodide (180 mg, 1.20 mmol) in DMF (4 mL) was sealed and heated in a microwave system at 85° C. for 4 h. The reaction mixture was cooled to rt, poured into water (50 mL) and extracted with EtOAc (20 mL). The organic component was washed with brine, dried over MgSO₄, filtered and concentrated. The residue was purified by FCC (40 g silica gel cartridge, eluted with gradient 10%˜50% EtOAc-Hexanes) to afford N-(2-fluoroethyl)-4-methoxyaniline (115 mg) as a light yellow oil. ¹H NMR (400 MHZ, CDCl₃) δ ppm 6.92-6.76 (m, 2H), 6.72-6.59 (m, 2H), 4.77-4.66 (m, 1H), 4.64-4.54 (m, 1H), 3.78-3.76 (m, 4H), 3.45 (t, J=4.9 Hz, 1H), 3.39 (t, J=4.9 Hz, 1H).

Step 2.

To a solution of N-(2-fluoroethyl)-4-methoxyaniline (170 mg, 1.00 mmol) and (S)-2-((tert-butoxycarbonyl)amino)-3-(3,5-difluorophenyl)propanoic acid (333 mg, 1.10 mmol) was added DIPEA (0.70 mL, 4.02 mmol) and HATU (420 mg, 1.10 mmol) and the reaction mixture was stirred at rt overnight. The reaction mixture was poured into water (80 mL) and extracted with EtOAc (50 mL). The organic component was washed with water, 5% citric acid and brine, dried over MgSO₄, filtered and concentrated. The residue was purified by FCC (80 g silica gel cartridge, eluted with gradient 20%-50% EtOAc-hexanes) to afford (S)-tert-butyl (3-(3,5-difluorophenyl)-1-((2-fluoroethyl)(4-methoxyphenyl)amino)-1-oxopropan-2-yl)carbamate (350 mg) as a white foam. LC-MS retention time=1.31 min; m/z=453.1 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

Step 3.

A mixture of (S)-tert-butyl (3-(3,5-difluorophenyl)-1-((2-fluoroethyl)(4-methoxyphenyl)amino)-1-oxopropan-2-yl)carbamate (300 mg, 0.663 mmol) and 4 M hydrogen chloride in 1,4-dioxane (3.32 mL, 13.3 mmol) was stirred at rt overnight. The reaction mixture was concentrated and the residue was treated with ether (5 mL). The residual gum was decanted with ether and dried in vacuo to afford an HCl salt of the title compound (225 mg) as a light yellow solid. LC-MS retention time=0.90 min; m/z=353.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm). ¹H NMR (400 MHZ, methanol-d₄) δ ppm 7.55-6.71 (m, 5H), 6.53 (d, J=6.0 Hz, 2H), 4.71-4.44 (m, 2H), 4.05 (d, J=18.1 Hz, 2H), 3.96 (br. s., 1H), 3.86 (s, 3H), 3.12 (d, J=8.5 Hz, 1H), 2.91 (d, J=7.5 Hz, 1H).

### Intermediate 68

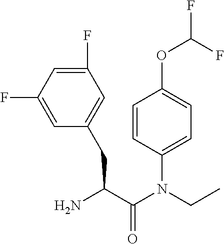 [SMILES: CCN(C(=O)[C@@H](N)Cc1cc(F)cc(F)c1)c1ccc(OC(F)F)cc1]

Step 1.

A mixture of 4-(difluoromethoxy)aniline (795 mg, 5.00 mmol), ethyl iodide (0.44 mL, 5.50 mmol) and cesium carbonate (1.95 g, 5.99 mmol) in DMF (20 mL) was sealed and heated in a microwave system at 85° C. for 2 h. The reaction mixture was diluted it with EtOAc (50 mL) and poured into water (100 mL). The organic component was washed with brine, dried over MgSO₄, filtered and concentrated in vacuo. The residue was purified by FCC (80 g silica gel cartridge, eluted with gradient 5%˜40% EtOAc-hexanes) to afford 4-(difluoromethoxy)-N-ethylaniline (445 mg) as a colorless oil. ¹H NMR (400 MHZ, DMSO-d₆) δ ppm 7.13, 6.94, 6.75 (t, 1H), 7.00-6.85 (m, 2H), 6.55 (d, J=8.8 Hz, 2H), 5.58 (t, J=5.1 Hz, 1H), 3.12-2.91 (m, 2H), 1.28-1.07 (m, 3H).

Step 2.

To a solution of 4-(difluoromethoxy)-N-ethylaniline (354 mg, 1.89 mmol) and (S)-2-((tert-butoxycarbonyl)amino)-3-(3,5-difluorophenyl)propanoic acid (518 mg, 1.72 mmol) in DMF (10 mL) was added DIPEA (1.20 mL, 6.88 mmol) and HATU (719 mg, 1.89 mmol). The reaction mixture was stirred at rt for 4 h, poured into water (100 mL) and extracted with DCM (20 mL×2). The organic component was washed with 5% citric acid and brine, dried over MgSO₄, filtered and concentrated in vacuo. The residue was purified by FCC (80 g silica gel cartridge, eluted with gradient 0˜50% EtOAc-Hexanes) to afford (S)-tert-butyl(1-((4-(difluoromethoxy)phenyl)(ethyl)amino)-3-(3,5-difluorophenyl)-1-oxopropan-2-yl)carbamate (310 mg) as a white gum. LC-MS retention time=1.36 min; m/z=471.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

Step 3.

(S)-tert-butyl (1-((4-(difluoromethoxy)phenyl)(ethyl)amino)-3-(3,5-difluorophenyl)-1-oxopropan-2-yl)carbamate (310 mg, 0.659 mmol) was stirred in 4 M hydrogen chloride in 1,4-dioxane (3.30 mL, 13.2 mmol) at rt for 2 h and then the reaction mixture was concentrated. The residual off-white solid was triturated with ether (15 mL), filtered and dried in vacuo to afford an HCl salt of Intermediate 68 (245 mg) as a white powder. LC-MS retention time=0.96 min; m/z=371.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Intermediate 69

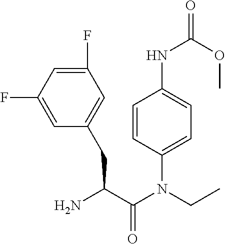 [SMILES: CCN(C(=O)[C@@H](N)Cc1cc(F)cc(F)c1)c1ccc(NC(=O)OC)cc1]

Step 1.

A slurry of 1-isocyanato-4-nitrobenzene (1.64 g, 9.99 mmol) and 20% Pd/C (0.053 g, 0.100 mmol) in THF (30 mL), MeOH (30 mL) and DCM (30 mL) was stirred under a H₂ balloon overnight. The reaction mixture was filtered and concentrated in vacuo and the residual oil was used without further purification. LC-MS retention time=0.62 min; m/z=167.1 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

Step 2.

A mixture of methyl (4-aminophenyl)carbamate (500 mg, 3.01 mmol), ethyl iodide (0.29 mL, 3.6 mmol) and cesium carbonate (1.27 g, 3.91 mmol) in DMF (15 mL) was sealed and heated in a microwave system at 85° C. for 2 h. The reaction mixture was diluted it with EtOAc (50 mL) and poured into water (150 mL). The organic component was washed with brine, dried over MgSO₄, filtered and concentrated in vacuo. The residue was purified by FCC (80 g silica gel cartridge, eluted with gradient 15%˜50% EtOAc-hexanes) to afford methyl (4-(ethylamino)phenyl)carbamate (257 mg) as a tan solid. LC-MS retention time=0.70 min; m/z=195.1 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm). ¹H NMR (400 MHZ, DMSO-d₆) δ ppm 9.12 (b, 1H), 7.12 (d, J=7.0 Hz, 2H), 6.60-6.39 (m, 2H), 5.24 (b, 1H), 3.60 (s, 3H), 2.97 (q, J=7.0 Hz, 2H), 1.13 (t, J=7.2 Hz, 3H).

Step 3.

To a solution of methyl (4-(ethylamino)phenyl)carbamate (200 mg, 1.03 mmol) and (S)-2-((tert-butoxycarbonyl)amino)-3-(3,5-difluorophenyl)propanoic acid (310 mg, 1.03 mmol) in DMF (5 mL) was added DIPEA (1.08 mL, 6.18 mmol) and HATU (431 mg, 1.13 mmol). The reaction mixture was stirred at rt overnight, poured into water (100 mL) and extracted with DCM (20 mL×2). The organic component was washed with 5% citric acid and brine, dried over MgSO₄, filtered and concentrated in vacuo. The residue was purified by FCC (80 g silica gel cartridge, eluted with gradient 0˜50% EtOAc-hexanes) to afford tert-butyl N-[(1S)-2-(3,5-difluorophenyl)-1-[ethyl({4[(methoxycarbonyl)amino]phenyl})carbamoyl]ethyl]carbamate as a white foam. LC-MS (Condition AW-1) retention time=1.22 min; m/z=478.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

Step 4.

A mixture of tert-butyl N-[(1S)-2-(3,5-difluorophenyl)-1-[ethyl({4-[(methoxycarbonyl)amino]phenyl})carbamoyl]ethyl]carbamate (285 mg, 0.597 mmol) and 4 M HCl in dioxane (2.98 mL, 11.9 mmol) was stirred at rt for 2 h. The reaction mixture was concentrated and the residual off-white solid was triturated with hexanes (15 mL), filtered and dried in vacuo to afford an HCl salt of Intermediate 69 (235 mg) as a white powder. LC-MS retention time=0.89 min; m/z=378.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm). ¹H NMR (400 MHZ, methanol-d₄) δ ppm 9.57 (s, 1H), 7.58 (br. s., 2H), 6.96 (tt, J=9.3, 2.3 Hz, 3H), 6.57 (dd, J=7.9, 2.1 Hz, 2H), 4.03 (t, J=7.0 Hz, 1H), 3.87 (dd, J=13.4, 7.2 Hz, 1H), 3.78 (s, 3H), 3.71-3.55 (m, 3H), 3.12 (dd, J=13.8, 7.0 Hz, 1H), 2.92 (dd, J=13.9, 7.2 Hz, 1H), 1.14 (t, J=7.2 Hz, 3H).

### Intermediate 70

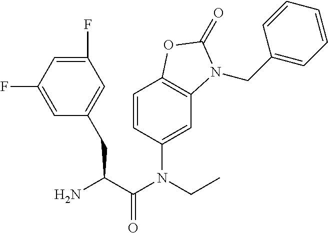 [SMILES: CCN(C(=O)[C@@H](N)Cc1cc(F)cc(F)c1)c1ccc2oc(=O)n(Cc3ccccc3)c2c1]

Step 1.

A mixture of 2-amino-4-nitrophenol (4.85 g, 31.5 mmol) and CDI (6.12 g, 37.8 mmol) in THF (50 mL) was heated at refluxing for 8 h. The reaction mixture was cooled to room temperature and concentrated. The residue was dissolved in DCM (100 mL) and poured into 2N HCl solution (200 mL) with stirring, after 30 min, the solid collected and washed with water (50 mL×2) and dried in vacuo to afford 5-nitrobenzo[d]oxazol-2(3H)-one (5.23 g) as a tan solid. ¹H NMR (400 MHZ, DMSO-d₆) δ ppm 8.05 (dd, J=8.8, 2.3 Hz, 1H), 7.86 (d, J=2.3 Hz, 1H), 7.52 (d, J=8.8 Hz, 1H).

Step 2.

A mixture of 5-nitrobenzo[d]oxazol-2(3H)-one (3.60 g, 20.0 mmol), benzyl bromide (2.61 mL, 22.0 mmol) and cesium carbonate (7.81 g, 23.98 mmol) in DMF (50 mL) was sealed and heated in a microwave system at 85° C. for 2 h. The reaction mixture was poured into water (500 mL), heated with a heat gun to near boiling and allowed to cool to rt with stirring. The resulting solids were collected by filtration, washed with water and hexanes and dried in vacuo to afford 3-benzyl-5-nitrobenzo[d]oxazol-2(3H)-one (5.20 g) as a gray solid. ¹H NMR (400 MHZ, DMSO-d₆) δ ppm 8.22-8.06 (m, 2H), 7.63 (d, J=8.8 Hz, 1H), 7.50-7.20 (m, 5H), 5.17 (s, 2H).

Step 3.

To a solution of 3-benzyl-5-nitrobenzo[d]oxazol-2(3H)-one (4.70 g, 17.4 mmol) in MeOH (25 mL) and DCM (100 mL) was added 10% Pd/C (0.555 g, 0.522 mmol). The reaction mixture was placed on a Parr shaker under a H₂ (30 psi) for 3 h, and then filtered and concentrated in vacuo to afford 5-amino-3-benzylbenzo[d]oxazol-2(3H)-one (4.05 g) as a white solid. ¹H NMR (400 MHZ, DMSO-d₆) δ ppm 7.48-7.24 (m, 5H), 7.00 (d, J=8.5 Hz, 1H), 6.38-6.20 (m, 2H), 5.05 (br. s., 2H), 4.93 (s, 2H).

Step 4.

A mixture of 5-amino-3-benzylbenzo[d]oxazol-2(3H)-one (800 mg, 3.33 mmol), ethyl iodide (0.32 mL, 4.00 mmol) and cesium carbonate (1.41 g, 4.33 mmol) in DMF (15 mL) was sealed and heated in a microwave system at 85° C. for 2 h. The reaction mixture was diluted it with EtOAc (50 mL) and poured into water (150 mL). The organic component was washed with brine, dried over MgSO₄, filtered and concentrated in vacuo. The residue was purified by FCC (80 g silica gel cartridge, eluted with gradient 10%˜50% EtOAc-hexanes) to afford 3-benzyl-5-(ethylamino)benzo[d]oxazol-2(3H)-one (450 mg) as a off-white solid. LC-MS retention time=0.88 min; m/z=269.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm). ¹H NMR (400 MHZ, DMSO-d₆) δ ppm 7.48-7.25 (m, 5H), 7.05 (d, J=8.8 Hz, 1H), 6.36 (d, J=2.3 Hz, 1H), 6.27 (dd, J=8.8, 2.3 Hz, 1H), 5.55 (t, J=5.4 Hz, 1H), 4.98 (s, 2H), 3.11-2.85 (m, 2H), 1.12 (t, J=7.2 Hz, 3H).

Step 5.

To a solution of 3-benzyl-5-(ethylamino)benzo[d]oxazol-2(3H)-one (305 mg, 1.14 mmol) and (S)-2-((tert-butoxycarbonyl)amino)-3-(3,5-difluorophenyl)propanoic acid (342 mg, 1.14 mmol) in DMF (10 mL) was added DIPEA (1.2 mL, 6.8 mmol) and HATU (475 mg, 1.25 mmol). The reaction mixture was stirred at rt overnight, poured water (100 mL) and extracted with DCM (20 mL×2). The organic components was washed with 5% citric acid and brine, dried over MgSO₄, filtered and concentrated in vacuo. The residue was purified by FCC (80 g silica gel cartridge, eluted with gradient 20%˜50% EtOAc-hexanes) to afford (S)-tert-butyl (1-((3-benzyl-2-oxo-2,3-dihydrobenzo[d]oxazol-5-yl)(ethyl)amino)-3-(3,5-difluorophenyl)-1-oxopropan-2-yl)carbamate (180 mg) as a white foam. LC-MS retention time=1.39 min; m/z=452.2 [M-Boc+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

Step 6.

A mixture of (S)-tert-butyl (1-((3-benzyl-2-oxo-2,3-dihydrobenzo[d]oxazol-5-yl)(ethyl)amino)-3-(3,5-difluorophenyl)-1-oxopropan-2-yl)carbamate (30 mg, 0.054 mmol) and 4 M HCl in dioxane (0.27 mL, 1.09 mmol) was stirred at rt for 2 h. The reaction mixture was concentrated and the residue was used without further purification. LC-MS retention time=0.83 min; m/z=452.1 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Intermediate 71

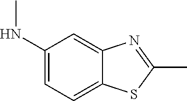

A solution of 25% w/w NaOMe in MeOH (0.87 mL, 3.8 mmol) was added to a stirred mixture of paraformaldehyde (46 mg, 1.5 mmol) and 2-methylbenzo[d]thiazol-5-amine (126 mg, 0.76 mmol) in MeOH (5 mL) and the reaction mixture was stirred at 60° C. for 16 h. The reaction mixture was cooled to rt, treated with NaBH₄ (72.4 mg, 1.91 mmol) and stirred at rt for 16 h. The reaction mixture was treated with 1N aq NaOH (4 mL), extracted with chloroform (10 mL×3) and the combined organic component was dried, concentrated and purified by flash silica chromatography (12 g SiO₂, 0-50% EtOAc/Hexanes) to yield Intermediate 71 (130 mg) as yellow solid. .LC-MS retention time=0.74 min; m/z=179.0 [M+H]⁺. (Column: Waters Aquity BEH C18, 2.1×50 mm, 1.7-μm particles. Solvent A=100% Water: 0.05% TFA. Solvent B=100% Acetonitrile: 0.05% TFA. Flow Rate=0.8 mL/min. Start % B=2. Final % B=98. Gradient Time=1.5 min. Wavelength=220). ¹H NMR (400 MHZ, CDCl₃) δ ppm 7.56 (d, J=8.6 Hz, 1H), 7.16 (d, J=2.3 Hz, 1H), 6.72 (dd, J=8.6, 2.3 Hz, 1H), 3.86 (br. s., 1H), 2.91 (s, 3H), 2.80 (s, 3H).

### Intermediate 72

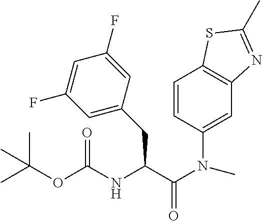 [SMILES: Cc1nc2cc(N(C)C(=O)[C@H](Cc3cc(F)cc(F)c3)NC(=O)OC(C)(C)C)ccc2s1]

HATU (128 mg, 0.34 mmol) was added to a stirred solution of Intermediate 71 (50 mg, 0.28 mmol) and (S)-2-((tert-butoxycarbonyl)amino)-3-(3,5-difluorophenyl)propanoic acid (101 mg, 0.34 mmol) in DMF (1 mL) and DIPEA (0.10 mL, 0.56 mmol) and the reaction mixture was stirred at rt for 16 h. The crude reaction was concentrated, and then purified with silica chromatography (12 g SiO₂, 0-50% EtOAc/hexanes) to yield Intermediate 72 (153 mg) as a mixture (3:1) with Intermediate 71. The mixture was carried through next step without further purification. LC-MS retention time=1.27 min; m/z=462.1 [M+H]⁺. (Column: Waters Aquity BEH C18, 2.1×50 mm, 1.7-μm particles. Solvent A=100% Water: 0.05% TFA. Solvent B=100% Acetonitrile: 0.05% TFA. Flow Rate=0.8 mL/min. Start % B=2. Final % B=98. Gradient Time=1.5 min. Wavelength=220).

### Intermediate 73

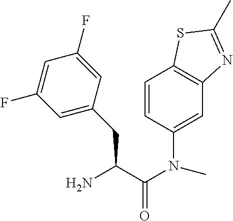 [SMILES: Cc1nc2cc(N(C)C(=O)[C@@H](N)Cc3cc(F)cc(F)c3)ccc2s1]

A solution of 4M HCl (1.5 mL, 6.0 mmol) in 1,4-dioxane was added to a mixture of Intermediate 72 (0.153 g, 0.25 mmol) in MeOH (1.5 mL) and the reaction mixture was stirred at rt for 16 h. The reaction mixture was concentrated and the residue was azeotroped with EtOH and ACN to afford an HCl salt of Intermediate 73 (126 mg) as white solid. LC-MS retention time=0.88 min; m/z=362.0 [M+H]⁺. (Column: Waters Aquity BEH C18, 2.1×50 mm, 1.7-μm particles. Solvent A=100% Water: 0.05% TFA. Solvent B=100% Acetonitrile: 0.05% TFA. Flow Rate=0.8 mL/min. Start % B=2. Final % B=98. Gradient Time=1.5 min. Wavelength=220). ¹H NMR (400 MHZ, methanol-d₄) δ ppm 8.05 (d, J=8.5 Hz, 1H), 7.68-7.51 (m, 1H), 7.15-6.96 (m, 1H), 6.97-6.88 (m, 1H), 6.55-6.46 (m, 2H), 4.23-4.14 (m, 1H), 3.36 (s, 3H), 3.16-2.92 (m, 2H), 2.90 (s, 3H).

### Intermediate 74

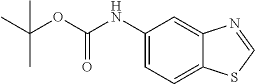 [SMILES: CC(C)(C)OC(=O)Nc1ccc2scnc2c1]

Boc₂O (0.51 mL, 2.2 mmol) and then DMAP (49 mg, 0.4 mmol) were added to a solution of benzo[d]thiazol-5-amine (300 mg, 2.0 mmol) in THF (5 mL) and the reaction mixture was stirred at 68° C. for 16 h. The reaction mixture was filtered, concentrated and purified by flash silica chromatography (0-30% EtOAc/hexanes) to yield Intermediate 74 (447 mg) as white solid. LC-MS retention time=1.08 min; m/z=251.0 [M+H]⁺. (Column: Waters Aquity BEH C18, 2.1×50 mm, 1.7-μm particles. Solvent A=100% Water: 0.05% TFA. Solvent B=100% Acetonitrile: 0.05% TFA. Flow Rate=0.8 mL/min. Start % B=2. Final % B=98. Gradient Time=1.5 min. Wavelength=220). ¹H NMR (400 MHZ, CDCl₃) δ ppm 9.00 (s, 1H), 8.10 (d, J=2.0 Hz, 1H), 7.86 (d, J=8.8 Hz, 1H), 7.67-7.52 (m, 1H), 6.69 (br. s., 1H), 1.56 (s, 9H).

### Intermediate 75

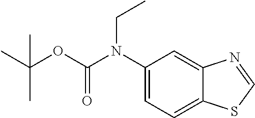 [SMILES: CCN(C(=O)OC(C)(C)C)c1ccc2scnc2c1]

Cs₂CO₃ (0.39 g, 1.2 mmol) was added to a solution of Intermediate 74 (0.2 g, 0.8 mmol) and bromoethane (0.09 mL, 1.2 mmol) in DMF (5 mL) and the reaction mixture was stirred at 70° C. for 2 h and then at rt for 2 d. Additional bromoethane (0.09 mL, 1.2 mmol) and Cs₂CO₃ (0.39 g, 1.2 mmol) was added and the reaction mixture was stirred at 70° C. for 2 h. Still more bromoethane (0.09 mL, 1.2 mmol) and Cs₂CO₃ (0.39 g, 1.2 mmol) was added and the reaction mixture was stirred at 70° C. for 2 h. The reaction was quenched by sat. aq. NH₄Cl, extracted with EtOAc (2×10 mL) and concentrated. The crude residue was dissolved in DMF (5 mL), treated with bromoethane (0.09 mL, 1.2 mmol), and Cs₂CO₃ (0.39 g, 1.2 mmol) and stirred at 70° C. for 3 h. The reaction was quenched by sat. aq. NH₄Cl (10 mL), extracted with EtOAc (2×10 mL) concentrated and purified by flash silica gel chromatography (12 g SiO₂, 0-33% EtOAc/hexanes) to yield Intermediate 75 (0.21 g) as yellow gum. LC-MS retention time=1.23 min; m/z=279.2 [M+H]⁺. (Column: Waters Aquity BEH C18, 2.1×50 mm, 1.7-μm particles. Solvent A=100% Water: 0.05% TFA. Solvent B=100% Acetonitrile: 0.05% TFA. Flow Rate=0.8 mL/min. Start % B=2. Final % B=98. Gradient Time=1.5 min. Wavelength=220). ¹H NMR (400 MHZ, methanol-d₄) δ ppm 9.30 (s, 1H), 8.07 (d, J=8.5 Hz, 1H), 7.91 (d, J=1.9 Hz, 1H), 7.38 (dd, J=8.5, 1.9 Hz, 1H), 3.77 (q, J=7.0 Hz, 2H), 1.55-1.35 (m, 9H), 1.18 (t, J=7.0 Hz, 3H).

### Intermediate 76

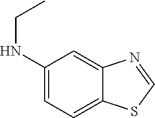

A solution of 4M HCl (2.0 mL, 8.0 mmol) in 1,4-dioxane was added to a solution of Intermediate 75 (0.21 g, 0.75 mmol) in MeOH (2 mL) and the reaction mixture was stirred at rt for 2 h. The reaction mixture was concentrated and the residue was azeotroped with EtOH to yield an HCl salt of Intermediate 76 (189 mg) as yellow solid. LC-MS retention time=0.72 min; m/z=179.0 [M+H]⁺. (Column: Waters Aquity BEH C18, 2.1×50 mm, 1.7-μm particles. Solvent A=100% Water: 0.05% TFA. Solvent B=100% Acetonitrile: 0.05% TFA. Flow Rate=0.8 mL/min. Start % B=2. Final % B=98. Gradient Time=1.5 min. Wavelength=220). ¹H NMR (400 MHZ, methanol-d₄) δ ppm 9.47 (s, 1H), 8.34 (d, J=8 Hz, 1H), 8.22 (s, 1H), 7.63 (d, J=8 Hz, 1H), 3.57 (q, J=7.0 Hz, 2H), 1.42 (t, J=7.0 Hz, 3H).

### Intermediate 77

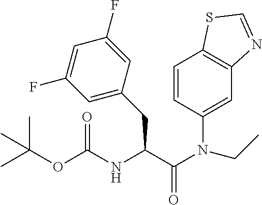 [SMILES: CCN(C(=O)[C@H](Cc1cc(F)cc(F)c1)NC(=O)OC(C)(C)C)c1ccc2scnc2c1]

HATU (109 mg, 0.29 mmol) was added to a solution of an HCl salt of Intermediate 76 (60 mg, 0.24 mmol) and (S)-2-((tert-butoxycarbonyl)amino)-3-(3,5-difluorophenyl)propanoic acid (86 mg, 0.29 mmol) in DMF (2 mL) and DIPEA (0.21 mL, 1.2 mmol) and the reaction mixture was stirred at rt for 16 h. The crude reaction was concentrated and then purified with silica chromatography (12 g SiO₂, 0-50% EtOAc/hexanes) to yield Intermediate 77 (71 mg) as a white solid. LC-MS retention time=1.27 min; m/z=462.2 [M+H]⁺. (Column: Waters Aquity BEH C18, 2.1×50 mm, 1.7-μm particles. Solvent A=100% Water: 0.05% TFA. Solvent B=100% Acetonitrile: 0.05% TFA. Flow Rate=0.8 mL/min. Start % B=2. Final % B=98. Gradient Time=1.5 min. Wavelength=220).

### Intermediate 78

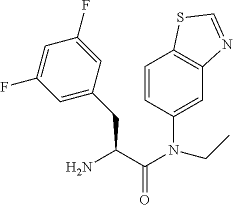 [SMILES: CCN(C(=O)[C@@H](N)Cc1cc(F)cc(F)c1)c1ccc2scnc2c1]

A solution of 4M HCl (1.0 mL, 4.0 mmol) in 1,4-dioxane was added to a solution of Intermediate 77 (71 mg, 0.15 mmol) in MeOH (1 mL) and the reaction mixture was stirred at rt for 4 h. The reaction mixture was concentrated and the residue was azeotroped with EtOH to give an HCl salt of Intermediate 78 (69 mg) as pink solid. LC-MS retention time=0.89 min; m/z=362.0 [M+H]⁺. (Column: Waters Aquity BEH C18, 2.1×50 mm, 1.7-μm particles. Solvent A=100% Water: 0.05% TFA. Solvent B=100% Acetonitrile: 0.05% TFA. Flow Rate=0.8 mL/min. Start % B=2. Final % B=98. Gradient Time=1.5 min. Wavelength=220).

### Intermediate 80

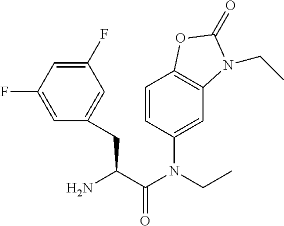 [SMILES: CCN(C(=O)[C@@H](N)Cc1cc(F)cc(F)c1)c1ccc2oc(=O)n(CC)c2c1]

Step 1.

A mixture of 2-amino-4-nitrophenol (4.85 g, 31.5 mmol) and CDI (6.12 g, 37.8 mmol) in THF (50 mL) was heated at reflux for 8 h, then allowed to cool to room temperature and the solvent was removed under reduced pressure. The residue was dissolved in DCM (100 mL) and poured into 2N HCl solution (200 mL) with stirring. After 30 min, the resulting solid was collected by filtration, washed with water (2×50 mL) and dried in vacuo to afford 5-nitrobenzo[d]oxazol-2(3H)-one (5.23 g) as a tan solid. ¹H NMR (400 MHZ, DMSO-d₆) δ 8.05 (dd, J=8.8, 2.3 Hz, 1H), 7.86 (d, J=2.3 Hz, 1H), 7.52 (d, J=8.8 Hz, 1H).

Step 2.

To a solution of 5-nitrobenzo[d]oxazol-2(3H)-one (1.67 g, 9.27 mmol) in MeOH (50 mL) and DCM (50 mL) was added 10% palladium/C (0.247 g, 0.232 mmol). The reaction mixture was placed on a Parr shaker under H₂ (30 psi) overnight. The suspension was filtered and evaporated in vacuo to afford 5-aminobenzo[d]oxazol-2(3H)-one (1.35 g) as a white solid. ¹H NMR (500 MHZ, DMSO-d₆) δ 11.20 (br. s., 1H), 6.91 (d, J=8.5 Hz, 1H), 6.33 (d, J=2.0 Hz, 1H), 6.25 (dd, J=8.4, 2.1 Hz, 1H), 5.23 (br. s., 2H).

Step 3.

A mixture of 5-aminobenzo[d]oxazol-2(3H)-one (500 mg, 3.33 mmol), ethyl iodide (0.323 mL, 4.00 mmol) and cesium carbonate (1.41 g, 4.33 mmol) in DMF (20 mL) was sealed and heated under microwave radiation at 85° C. for 2 h. The reaction mixture was diluted with EtOAc (50 mL) and poured into water (150 mL). The separated organic component was washed with brine, dried over MgSO₄, filtered and evaporated in vacuo. The residue was purified by FCC (80 g silica gel cartridge), eluting with gradient 20%˜70% EtOAc-hexanes to afford 3-ethyl-5-(ethylamino)benzo[d]oxazol-2(3H)-one (155 mg) as a colorless oil. ¹H NMR (400 MHZ, DMSO-d₆) δ 7.03 (d, J=8.5 Hz, 1H), 6.45 (d, J=2.0 Hz, 1H), 6.29 (dd, J=8.5, 2.3 Hz, 1H), 5.54 (t, J=5.4 Hz, 1H), 3.78 (q, J=7.2 Hz, 2H), 3.17-2.95 (m, 2H), 1.23 (t, J=7.2 Hz, 3H), 1.17 (t, J=7.2 Hz, 3H).

Step 4.

To a solution of 3-ethyl-5-(ethylamino)benzo[d]oxazol-2(3H)-one (311 mg, 1.51 mmol) and (S)-2-((tert-butoxycarbonyl)amino)-3-(3,5-difluorophenyl)propanoic acid (454 mg, 1.51 mmol) was added DIPEA (1.58 mL, 9.05 mmol) and HATU (631 mg, 1.66 mmol) and the reaction mixture was stirred at rt ON. The reaction mixture was poured into water (100 mL) and extracted with DCM (2×20 mL). The combined organic component was washed with 5% citric acid and brine, dried over MgSO₄, filtered and evaporated in vacuo. The residue was purified by preparative HPLC to afford (S)-tert-butyl (3-(3,5-difluorophenyl)-1-(ethyl(3-ethyl-2-oxo-2,3-dihydrobenzo[d]oxazol-5-yl)amino)-1-oxopropan-2-yl)carbamate (180 mg) as a white foam. LC-MS retention time=1.30 min; m/z=490.25 [M+H]+ (Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7 μm, Oven Temp.=40° C.).

Step 5.

A mixture of (S)-tert-butyl(3-(3,5-difluorophenyl)-1-(ethyl(3-ethyl-2-oxo-2,3-dihydrobenzo[d]oxazol-5-yl)amino)-1-oxopropan-2-yl)carbamate (350 mg, 0.715 mmol) and 4 M HCl in dioxane (3.58 mL, 14.3 mmol) was stirred at rt for 2 h. The solvent was removed by a steady stream of nitrogen. The residue was triturated with ether, filtered, washed with ether and dried in vacuo to afford an HCl salt of the title compound as a purple powder. LC-MS retention time=0.91 min; m/z=390.00 [M+H]+ (Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7 U, MW1=132+ Oven Temp.=40° C.).

### Intermediate 81

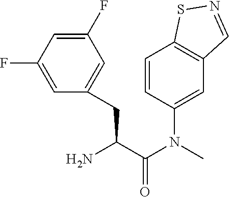 [SMILES: CN(C(=O)[C@@H](N)Cc1cc(F)cc(F)c1)c1ccc2sncc2c1]

Step 1.

A mixture of benzo[d]isothiazol-5-amine (500 mg, 3.33 mmol) and N,N-dimethylformamide dimethyl acetal (2.45 mL, 18.3 mmol) was sealed and heated in an oil bath at 110° C. for 2 h. The solvent was removed by evaporation and the residual tar was taken up into MeOH (10 mL) and DMF (10 mL). To this solution was added sodium borohydride (630 mg, 16.6 mmol) and the reaction mixture was stirred at rt for 1 h, diluted with EtOAc (50 mL) and poured into water (150 mL). The separated organic component was washed with brine, dried over MgSO₄, filtered and evaporated in vacuo. The residue was purified by FCC (80 g silica gel cartridge), eluting with gradient 20%˜70% EtOAc-hexanes to afford N-methylbenzo[d]isothiazol-5-amine (125 mg) as a light yellow solid. ¹H NMR (500 MHZ, DMSO-d₆) δ 8.86 (d, J=0.5 Hz, 1H), 7.84 (d, J=8.8 Hz, 1H), 7.07 (d, J=2.0 Hz, 1H), 7.00 (dd, J=8.8, 2.0 Hz, 1H), 5.99 (d, J=4.8 Hz, 1H), 2.75 (d, J=5.0 Hz, 3H).

Step 2.

To a solution of N-methylbenzo[d]isothiazol-5-amine (120 mg, 0.731 mmol) and (S)-2-((tert-butoxycarbonyl)amino)-3-(3,5-difluorophenyl)propanoic acid (220 mg, 0.731 mmol) was added DIPEA (0.766 mL, 4.38 mmol) and HATU (306 mg, 0.804 mmol). The resulting mixture was stirred at rt for 4 h, poured into water (100 mL) and extracted with DCM (2×20 mL). The combined organic components were washed with 5% citric acid and brine, dried over MgSO₄, filtered and evaporated in vacuo. The residue was purified by preparative HPLC to afford (S)-tert-butyl (1-(benzo[d]isothiazol-5-yl(methyl)amino)-3-(3,5-difluorophenyl)-1-oxopropan-2-yl)carbamate (250 mg) as an off-white foam. LC-MS retention time=1.27 min; m/z=391.90 [M-^{t}Bu]+ (Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7 μm, Oven Temp.=40° C.).

Step 3.

A mixture of (S)-tert-butyl(1-(benzo[d]isothiazol-5-yl(methyl)amino)-3-(3,5-difluorophenyl)-1-oxopropan-2-yl)carbamate (250 mg, 0.559 mmol) and 4 M HCl in dioxane (2.79 mL, 11.2 mmol) was stirred at rt for 2 h. The solvent was removed by evaporation and the residue was triturated with ether, filtered, washed with ether and dried in vacuo to afford an HCl salt of the title compound (180 mg) as a tan powder. LC-MS retention time=0.87 min; m/z=348.10 [M+H]+ (Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7 μm, Oven Temp.=40° C.). ¹H NMR (500 MHZ,methanol-d₄) δ 8.99 (s, 1H), 8.21 (d, J=8.7 Hz, 1H), 7.90-7.40 (m, 2H), 6.98 (t, J=9.1 Hz, 1H), 6.53 (d, J=6.1 Hz, 2H), 4.17 (t, J=7.0 Hz, 1H), 3.37 (s, 3H), 3.12 (dd, J=13.7, 7.9 Hz, 1H), 2.97 (dd, J=13.6, 6.4 Hz, 1H).

### Intermediate 82

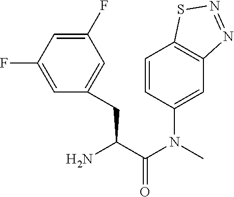 [SMILES: CN(C(=O)[C@@H](N)Cc1cc(F)cc(F)c1)c1ccc2snnc2c1]

Step 1.

To a solution of methyl benzo[d][1,2,3]thiadiazole-5-carboxylate (1.98 g, 10.2 mmol) in THF (25 mL) and MeOH (25 mL) was added a solution of lithium hydroxide monohydrate (0.86 g, 20 mmol) in water (25 mL). The reaction mixture was stirred at rt ON and then partially concentrated (to ˜⅓ volume) in vacuo, diluted with water (25 mL), cooled with an ice bath and acidified with 5% citric acid to pH 2. The formed precipitate was collected by filteratio, washed with water and dried in vacuo to afford benzo[d][1,2,3]thiadiazole-5-carboxylic acid (1.82 g) as a white solid. ¹H NMR (500 MHZ, DMSO-d₆) δ 13.56 (br. s., 1H), 9.17 (s, 1H), 8.55 (d, J=8.5 Hz, 1H), 8.31 (d, J=8.5 Hz, 1H).

Step 2.

To a mixture of benzo[d][1,2,3]thiadiazole-5-carboxylic acid (541 mg, 3.00 mmol) in toluene (30 mL) was added TEA (0.54 mL, 3.9 mmol) and diphenyl phosphorazidate (0.78 mL, 3.60 mmol). The reaction mixture was sealed and heated to 45° C. for 2 h. Tert-butyl alcohol (2.87 mL, 30.0 mmol) was added, the reaction vessel was resealed and heating continued at 110° C. for 8 h. The reaction mixture was cooled, filtered to remove solids and the filtrate was concentrated in vacuo. The residue was taken up into EtOAc (50 mL), washed with sat. NaHCO₃ and brine, dried over MgSO₄, filtered and evaporated in vacuo. The residue was taken up into DCM and purified by FCC (80 g silica gel cartridge), eluting with gradient 15%˜50% EtOAc-hexanes to afford tert-butyl benzo[d][1,2,3]thiadiazol-5-ylcarbamate (377 mg) as an off-white solid. ¹H NMR (500 MHZ, DMSO-d₆) δ 9.92 (br. s., 1H), 8.84 (s, 1H), 8.28 (d, J=8.8 Hz, 1H), 7.81 (d, J=8.8 Hz, 1H), 1.54 (s, 9H).

Step 3.

A mixture of tert-butyl benzo[d][1,2,3]thiadiazol-5-ylcarbamate (0.370 g, 1.47 mmol) and 4 M HCl in dioxane (9.20 mL, 36.8 mmol) was stirred at rt for 3 h and then the solvent was removed in vacuo. The residue was triturated with ether, filtered, washed with ether and dried in vacuo to afford benzo[d][1,2,3]thiadiazol-5-amine, HCl (250 mg) as a light yellow powder. LC-MS retention time=0.74 min; m/z=152.00 [M+H]+ (Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7 μm, Oven Temp.=40° C.).

Step 4.

To a mixture of benzo[d][1,2,3]thiadiazol-5-amine, HCl (247 mg, 1.32 mmol) and paraformaldehyde (79 mg, 2.6 mmol) in MeOH (8 mL) was added sodium methoxide (2.10 mL, 9.21 mmol) and the reaction mixture mixture was sealed and and heated at 55° C. for 2 h. After cooling to rt, sodium borohydride (124 mg, 3.29 mmol) was added in small portions and the final mixture was stirred at rt ON. The solvent was then removed in vacuo and the residue was taken up in EtOAc (20 mL) and washed with water (50 mL). The separated aqueous component was saturated with NaCl and extracted with EtOAc (20 mL). The combined organic components were washed with brine (25 mL), dried over MgSO₄, filtered, and concentrated in vacuo. The residue was taken up into DCM (5 mL) and purified by FCC (40 g silica gel cartridge), eluting with gradient 15%˜50% EtOAc-hexanes to afford N-methylbenzo[d][1,2,3]thiadiazol-5-amine (217 mg) as a yellow powder. ¹H NMR (500 MHZ, DMSO-d₆) δ 8.03 (d, J=8.8 Hz, 1H), 7.45 (d, J=1.9 Hz, 1H), 7.18 (dd, J=8.8, 2.0 Hz, 1H), 6.38 (d, J=4.6 Hz, 1H), 2.82 (d, J=4.9 Hz, 3H).

Step 5.

A mixture of (S)-2-((tert-butoxycarbonyl)amino)-3-(3,5-difluorophenyl)propanoic acid (401 mg, 1.33 mmol), N-methylbenzo[d][1,2,3]thiadiazol-6-amine (200 mg, 1.21 mmol) and N-ethoxycarbonyl-2-ethoxy-1,2-dihydroquinoline (329 mg, 1.33 mmol) in DCM (10 mL) was stirred at rt overnight. The solvent was removed in vacu and the residue was taken up into DCM (5 mL) and purified by FCC (80 g silica gel cartridge), eluting with gradient 15%˜60% EtOAc-hexanes to afford (S)-tert-butyl (1-(benzo[d][1,2,3]thiadiazol-5-yl(methyl)amino)-3-(3,5-difluorophenyl)-1-oxopropan-2-yl)carbamate (223 mg) as a white gum. ¹H NMR (500 MHZ, CDCl₃) δ 8.11 (d, J=8.4 Hz, 2H), 7.25 (b, 1H), 6.74 (t, J=8.8 Hz, 1H), 6.49 (d, J=5.0 Hz, 2H), 5.22 (d, J=7.9 Hz, 1H), 4.52 (d, J=6.5 Hz, 1H), 3.35 (s, 3H), 2.94 (dd, J=13.0, 8.4 Hz, 1H), 2.78 (dd, 5.8 Hz, 1H), 1.42 (s, 9H).

Step 6.

A mixture of (S)-tert-butyl (1-(benzo[d][1,2,3]thiadiazol-5-yl(methyl)amino)-3-(3,5-difluorophenyl)-1-oxopropan-2-yl)carbamate (220 mg, 0.491 mmol) and 4 M HCl in dioxane (2.45 mL, 9.81 mmol) was stirred at rt for 1 h. The solvent was removed by evaporation. The residue was triturated with ether, filtered, washed with ether and dried in vacuo to afford an HCl salt of the title compound Intermediate 82 (160 mg) as an off-white powder. LC-MS retention time=0.82 min; m/z=348.90 [M+H]+ (Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7 μm, Oven Temp.=40° C.).

### Intermediate 83

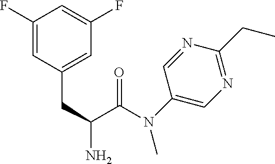 [SMILES: CCc1ncc(N(C)C(=O)[C@@H](N)Cc2cc(F)cc(F)c2)cn1]

Step 1.

To a mixture of 2-ethylpyrimidin-5-amine (1.07 g, 8.69 mmol) and paraformaldehyde (0.522 g, 17.4 mmol) in MeOH (35 mL) was added sodium methoxide (9.93 mL, 43.4 mmol). The resulting homogeneous solution was sealed and heated at 50° C. for 4 h. After cooling to rt, sodium borohydride (0.822 g, 21.7 mmol) was added in small portions and the final reaction mixture was stirred at rt overnight. The solvent was removed in vacuo and the residue was partitioned between DCM (50 mL) and water (50 mL). The separated aqueous component was extarcted with DCM (2×20 mL) and the combined organic components were washed with brine, dried over MgSO₄, filtered, and concentrated in vacuo. The residue was taken up into DCM (5 mL) and purified by FCC (80 g silica gel cartridge), eluting with gradient 15%˜50% acetone-hexanesto afford 2-ethyl-N-methylpyrimidin-5-amine (1.12 g) as a colorless oil. ¹H NMR (500 MHZ, DMSO-d₆) δ 8.03 (s, 2H), 5.83 (d, J=4.4 Hz, 1H), 2.77-2.66 (m, 5H), 1.20 (t, J=7.6 Hz, 3H).

Step 2.

A mixture of (S)-2-((tert-butoxycarbonyl)amino)-3-(3,5-difluorophenyl)propanoic acid (1.32 g, 4.37 mmol), 2-ethyl-N-methylpyrimidin-5-amine (600 mg, 4.37 mmol) and N-ethoxycarbonyl-2-ethoxy-1,2-dihydroquinoline (1.19 g, 4.81 mmol) in DCM (30 mL) was stirred at rt ON. The reaction mixture was then diluted with DCM (50 mL), washed with 5% citric acid and brine, dried over MgSO₄, filtered and concentrated in vacuo. The residue was taken up into DCM (5 mL), purified by FCC (80 g silica gel cartridge), eluting with gradient 15%˜100% EtOAc-hexanes to afford (S)-tert-butyl (3-(3,5-difluorophenyl)-1-((2-ethylpyrimidin-5-yl)(methyl)amino)-1-oxopropan-2-yl)carbamate (615 mg) as a white solid. LC-MS retention time=1.19 min; m/z=421.05 [M+H]+ (Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7 μm, Oven Temp.=40° C.).

Step 3.

A solution of (S)-tert-butyl (3-(3,5-difluorophenyl)-1-((2-ethylpyrimidin-5-yl)(methyl)amino)-1-oxopropan-2-yl)carbamate (540 mg, 1.28 mmol) in HCl (4N in dioxane) (6.42 mL, 25.7 mmol) was stirred at rt for 3 h. The solvent was evaporated in vacuo and the residue was triturated with ether (10 mL), filtered, washed with ether and dried in vacuo to afford an HCl salt of the title compound (479 mg) as an off-white solid. LC-MS retention time=0.71 min; m/z=321.30 [M+H]+ (Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7 μm, Oven Temp.=40° C.).

### Intermediate 86

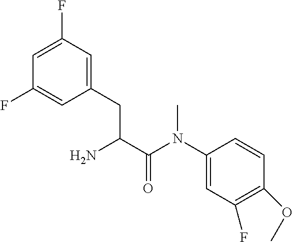 [SMILES: COc1ccc(N(C)C(=O)C(N)Cc2cc(F)cc(F)c2)cc1F]

Step 1.

To a mixture of 3-fluoro-4-methoxyaniline (3.00 g, 21.3 mmol) and paraformaldehyde (1.28 g, 42.5 mmol) in MeOH (100 mL) was added sodium methoxide (19.4 mL, 85.0 mmol) and the reaction mixture mixture was sealed and heated at 55° C. for 2 h. After cooling to rt, sodium borohydride (2.01 g, 53.1 mmol) was added in small portions. The final reaction mixture was stirred at rt overnight. The solvent was removed in vacuo and the residue was taken up in EtOAc (100 mL) and washed with water (150 mL). The separated aqueous component was saturated with NaCl, extracted with EtOAc (100 mL) and the combined organic components were washed with brine (50 mL), dried over MgSO₄, filtered, and concentrated in vacuo. The residue was taken up into DCM (10 mL) and purified by FCC (80 g silica gel cartridge), eluting with gradient 5%˜50% EtOAc-hexanes to afford 3-fluoro-4-methoxy-N-methylaniline (3.08 g) as a light yellow oil. LC-MS retention time=1.34 min; m/z=153.42 [M−H]−. (Start % B=0, Final % B=100, Gradient Time=2 min, Flow Rate=1 ml/min, Wavelength=220, Solvent Pair=Water: Acetonitrile 10 mM Ammonium Acetate, Solvent A=95% Water: 5% Acetonitrilel 10 mM Ammonium Acetate, Solvent B=5% Water: 95% Acetonitrile 10 mM Ammonium Acetate, Column=Phenomenex Luna C18 2.0×30 mm 3 μm, MW1=132+/− Oven Temp.=40° C.).

Step 2.

To a solution of (S)-2-((tert-butoxycarbonyl)amino)-3-(3,5-difluorophenyl)propanoic acid (971 mg, 3.22 mmol), 3-fluoro-4-methoxy-N-methylaniline (500 mg, 3.22 mmol) and HATU (1.35 g, 3.54 mmol) in DMF (15 mL) was added DIPEA (2.25 mL, 12.9 mmol) dropwise. The resulted yellow solution was stirred at rt ON. The mixture was then diluted with EtOAc (50 mL), poured into water (100 mL), separated and the aqueous component was saturated with NaCl and extracted with EtOAc (20 mL). The combined organic components were washed with brine, dried over MgSO₄, filtered and evaporated in vacuo. The residue was taken up into DCM (20 mL), purified by FCC (220 g silica gel cartridge), eluting with gradient 35%˜65% EtOAc-hexanes to afford (S)-tert-butyl (3-(3,5-difluorophenyl)-1-((3-fluoro-4-methoxyphenyl)(methyl)amino)-1-oxopropan-2-yl)carbamate (1.263 g) as a white foam. LC-MS retention time=1.30 min; m/z=439.05 [M+H]+. (Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7 μm, Oven Temp.=40° C.).

Step 3.

A mixture of (S)-tert-butyl (3-(3,5-difluorophenyl)-1-((3-fluoro-4-methoxyphenyl)(methyl)amino)-1-oxopropan-2-yl)carbamate (1.26 g, 2.88 mmol) and 4 M HCl in dioxane (5.76 mL, 23.0 mmol) was stirred at rt for 1 h. The solvent was removed in vacuo and dried in vacuo to afford an HCl salt of the title compound (1.10 g) as an off-white foam. LC-MS retention time=0.90 min; m/z=339.20 [M+H]+. (Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7 μm, Oven Temp.=40° C.). ¹H NMR (500 MHZ, methanol-d₄) δ 7.17 (t, J=8.9 Hz, 1H), 7.01-6.71 (m, 3H), 6.60 (d, J=6.3 Hz, 2H), 4.17 (t, J=7.1 Hz, 1H), 3.94 (s, 3H), 3.26 (s, 3H), 3.11 (dd, J=13.7, 7.6 Hz, 1H), 2.97 (dd, J=13.8, 6.7 Hz, 1H).

### Intermediate 87

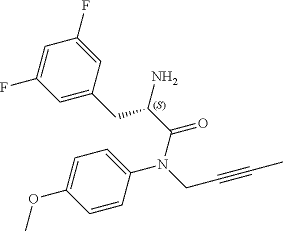

Step 1.

To an ice bath cooled solution of tert-butyl (4-methoxyphenyl)carbamate (2.23 g, 9.99 mmol) in DMF (50 mL) was added 60% NaH (0.439 g, 11.0 mmol) as a dispersion in mineral oil in small portions with vigorous stirring. After stirring at rt for 30 min, 1-bromobut-2-yne (0.96 mL, 11 mmol) was added and the reaction mixture was stirred at rt ON. The reaction was cooled in an ice bath, quenched with iced water (˜200 mL) and extracted with EtOAc (2×100 mL). The combined organic components were washed (brine), dried (MgSO₄), filtered and concentrated in vacuo. The residual oil was purified via Biotage (0%˜35% EtOAc/Hex; 80 g silica gel column) to afford tert-butyl but-2-yn-1-yl(4-methoxyphenyl)carbamate (2.50 g) as a white powder. LC-MS retention time=1.32 min; m/z=261.05 [M−CH₃]+ (Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7 μm, Oven Temp.=40° C.).

Step 2.

A mixture of tert-butyl but-2-yn-1-yl(4-methoxyphenyl)carbamate (2.50 g, 9.08 mmol) and 4 M HCl in 1,4-dioxane (11.4 mL, 45.4 mmol) was stirred at rt for 1 h. The solvent was removed in vacuo. The residue was triturated with ether (20 mL), filtered, washed with ether and dried in vacuo to afford an HCl salt of N-(but-2-yn-1-yl)-4-methoxyaniline (1.87) as an off-white powder. ¹H NMR (500 MHZ, methanol-d₄) δ 7.55-7.40 (m, 2H), 7.15-7.07 (m, 2H), 4.19 (q, J=2.4 Hz, 2H), 3.87 (s, 3H), 1.89 (t, J=2.4 Hz, 3H).

Step 3.

To a solution of an HCl salt of (S)-2-((tert-butoxycarbonyl)amino)-3-(3,5-difluorophenyl)propanoic acid (854 mg, 2.83 mmol), N-(but-2-yn-1-yl)-4-methoxyaniline (600 mg, 2.83 mmol) and HATU (1.18 g, 3.12 mmol) in DMF (20 mL) was added DIPEA (1.98 mL, 11.3 mmol) dropwise. The resulting yellow solution was stirred at rt for 3 h. The reaction mixture was then diluted with EtOAc (50 mL) and poured into water (150 mL). The separated aqueous component was saturated with NaCl and extracted with EtOAc (20 mL). The combined organic components were washed with brine, dried over MgSO₄, filtered and evaporated in vacuo. The residue was taken up into DCM (20 mL) and purified by FCC (120 g silica gel cartridge), eluting with gradient 10%˜50% EtOAc-hexanes to afford (S)-tert-butyl(1-(but-2-yn-1-yl(4-methoxyphenyl)amino)-3-(3,5-difluorophenyl)-1-oxopropan-2-yl)carbamate (1.08 g) as a white foam. LC-MS retention time=1.41 min; m/z=459.20 [M+H]+ (Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7 μm, Oven Temp.=40° C.).

Step 4.

A mixture of (S)-tert-butyl(1-(but-2-yn-1-yl(4-methoxyphenyl)amino)-3-(3,5-difluorophenyl)-1-oxopropan-2-yl)carbamate (1.00 g, 2.18 mmol) and 4 M HCl in 1,4-dioxane (4.91 mL, 19.6 mmol) was stirred at rt for 2 h. The solvent was removed in vacuo, triturated with ether, filtered, washed with ether and dried in vacuo to afford an HCl salt of Intermediate 87 (801 mg) as a white powder. LC-MS retention time=1.05 min; m/z=359.25 [M+H]+ (Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7 μm, Oven Temp.=40° C.). ¹H NMR (500 MHz, methanol-d₄) δ 7.18-6.87 (m, 5H), 6.65-6.49 (m, 2H), 4.52 (dd, J=17.0, 2.4 Hz, 1H), 4.31 (dd, J=16.9, 2.4 Hz, 1H), 4.07 (t, J=7.0 Hz, 1H), 3.87 (s, 3H), 3.12 (dd, J=13.9, 7.1 Hz, 1H), 2.93 (dd, J=13.9, 6.9 Hz, 1H), 1.79 (t, J=2.2 Hz, 3H).

### Intermediate 90

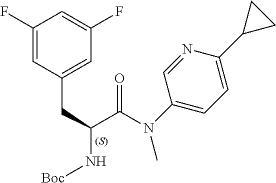

To a solution of (S)-2-((tert-butoxycarbonyl)amino)-3-(3,5-difluorophenyl)propanoic acid (0.549 g, 1.82 mmol) and 6-cyclopropyl-N-methylpyridin-3-amine (0.27 g, 1.8 mmol) in DMF (7 mL) was added DIPEA (0.636 mL, 3.64 mmol) followed by HATU (0.727 g, 1.91 mmol) and the reaction mixture was stirred at rt for 16 h. The reaction mixture was partitioned between water (70 mL) and EtOAc (35 mL), the organic component was dried with Na₂SO₄, and evaporated evaporated in vacuo to afford a light brown oil, which was purified further to afford the title product as an oil. LC-MS retention time=3.68 min; m/z=454.07 [M+Na]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, CDCl₃) δ 8.04 (s, 1H), 7.19-7.11 (m, 2H), 6.70 (t, J=8.8 Hz, 1H), 6.48 (d, J=5.9 Hz, 2H), 5.19 (m, 1H), 4.45 (m, 1H), 3.23 (s, 3H) 2.87 (m, 1H), 2.73 (m, 1H), 2.07 (m, 1H), 1.42 (s, 9H), 1.08 (m, 4H).

### Intermediate 91

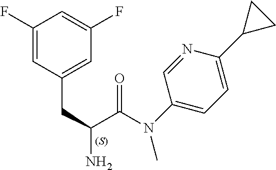

To a solution of Intermediate 90 (0.77 g, 1.8 mmol) in dioxane (1 mL) was added HCl (4N in dioxane) (1.50 mL, 6 mmol) and the cloudy solution was stirred at for 4 h. Methanol (1 mL) was added and the reaction mixture was stirred at rt for 16 h. HCl (4N in dioxane) (1 mL, 4 mmol) was added and the reaction mixture was stirred at rt for 4 h. The solvent was evaporated and dried under high vacuum to afford an HCl salt of the title compound (0.6 g) as a light grey solid. LC-MS retention time=2.74 min; m/z=332.07 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Intermediate 92

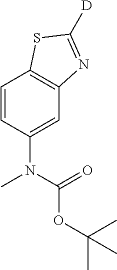 [SMILES: [2H]c1nc2cc(N(C)C(=O)OC(C)(C)C)ccc2s1]

To a solution of tert-butyl benzo[d]thiazol-5-yl(methyl)carbamate (0.88 g, 3.33 mmol) in THF (40 mL) was added N-butyllithium (2.5M in hexane) (2.26 mL, 5.66 mmol) at −78° C. and the reaction mixture was stirred for 30 min. Then D₂O (0.72 mL, 39.9 mmol) was added, and the reaction mixture was allowed to warm to rt and then was stirred at rt for 17 h. The solvent was evaporated and the residue was partitioned between water (20 mL) and EtOAc (20 mL). The organic component was separated, dried with Na₂SO₄, concentrated, and the residue was purified with a Biotage Horizon (10-80% EtOAc/Hexane) to afford the title compound (0.48 g). LC-MS retention time=2.63 min; m/z=210.20 [M+H]⁺ (the mass did not pick up). (Column: Phenomenex C18 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 8.12 (d, J=9.0 Hz, 1H), 7.99 (s, 1H), 7.44 (d, J=10.8 Hz, 1H), 3.34 (s, 3H), 1.41 (s, 9H).

### Intermediate 93

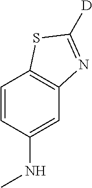

To a mixture of Intermediate 92 (0.46 g, 1.73 mmol) in dioxane (4 mL) was added HCl (4N in dioxane) (2.63 mL, 87 mmol) and the clear solution was stirred at rt for 17 h. The solvent was evaporated and dried under high vacuum for 64 h to afford the title compound (0.4 g) as a beige solid. LC-MS retention time=1.81 min; m/z=166.07 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 9.36 (s, 1H), 8.15 (d, J=2.5 Hz, 1H), 7.41 (d, J=2.5 Hz, 1H), 6.25 (m, 1H), 2.78 (d, J=5.0 Hz, 3H).

### Intermediate 94

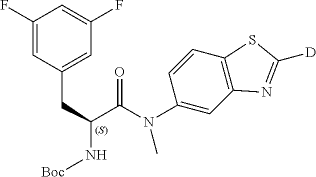

To a solution of (S)-2-((tert-butoxycarbonyl)amino)-3-(3,5-difluorophenyl)propanoic acid (0.523 g, 1.735 mmol) and Intermediate 93 (0.35 g, 1.74 mmol) in DMF (5 mL) was added DIPEA (0.76 mL, 4.34 mmol) and then HATU (0.69 g, 1.82 mmol) and the reaction mixture was stirred at rt for 3 h. The reaction mixture was partitioned between water (50 mL) and EtOAc (20 mL) and the organic component was separated and dried with Na₂SO₄, the solvent was evaporated and the crude material was purified with a Biotage Horizon (0-80% EtOAc/Hexane) to afford the title compound (0.50 g) as a light yellow foam. LC-MS retention time=3.85 min; m/z=471.21 [M+Na]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 8.32 (d, J=8.6 Hz, 1H), 8.12 (s, 1H), 7.49 (d, J=8.5 Hz, 1H), 7.22 (d, J=8.3 Hz, 1H), 6.97 (m, 1H), 6.48-6.38 (m, 2H), 4.30-4.16 (m, 1H), 3.26 (s, 3H), 2.89 (d, J=16.6 Hz, 1H), 2.71 (m, 1H), 1.27 (s, 9H).

### Intermediate 95

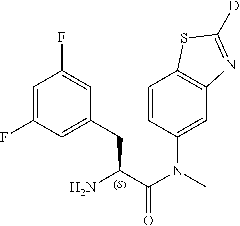

To a solution of Intermediate 94 (0.5 g, 1.12 mmol) in dioxane (3 mL) was added HCl (4N in dioxane) (1.69 mL, 55.7 mmol) and the reaction mixture was stirred at rt for 17 h. Methanol (1 mL) was added and the stirring was continued at rt for 5 h. The solvent was evaporated and dried under high vacuum overnight to afford the title compound (0.43 g) as a light yellow solid. LC-MS retention time=2.83 min; m/z=349.13 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Intermediate 100

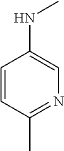

To a solution of 6-methylpyridin-3-amine (500 mg, 4.62 mmol) and formaldehyde (208 mg, 6.94 mmol) in methanol (10 mL) was added sodium methanolate (5.29 mL, 23.2 mmol) and the reaction mixture was heated to 50° C. for 16 h. The reaction mixture was cooled to rt, treated sodium tetrahydroborate (437 mg, 11.6 mmol) in two portions and stirred at rt for 6 h. Water (10 mL) was added slowly and the mixture was extracted by EtOAc (2×20 mL). The combined organic component was dried with Na₂SO₄, filtered, concentrated and purified using a Biotage (0-100% EtOAc, 10-20% MeOH/EtOAc) to afford the title compound (0.29 g) as a red oil. ¹H NMR (400 MHZ, CDCl₃-d) δ 7.97 (d, J=2.7 Hz, 1H), 6.98 (d, J=8.3 Hz, 1H), 6.85 (dd, J=8.3, 3.0 Hz, 1H), 2.86 (s, 3H), 2.46 (s, 3H).

### Intermediate 101

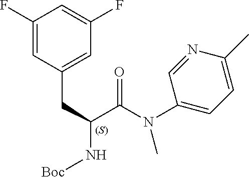

To a solution of (S)-2-((tert-butoxycarbonyl)amino)-3-(3,5-difluorophenyl)propanoic acid (0.715 g, 2.37 mmol) and Intermediate 100 (0.29 g, 2.4 mmol) in DMF (7 mL) was added DIPEA (0.83 mL, 4.75 mmol) and then HATU (0.95 g, 2.5 mmol) and the reaction mixture was stirred at rt for 2 h. The reaction mixture was partitioned between water (70 mL) and EtOAc (35 mL), the organic component was separated and dried with Na₂SO₄, filtered, concentrated and purified with a Biotage Horizon (20-90% EtOAc/Hexane) to afford the title compound (0.67 g) as off-white foam. LC-MS retention time=3.48 min; m/z=428.07 [M+Na]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, CDCl₃-d) δ 8.12 (s, 1H), 7.19 (d, J=8.0 Hz, 1H), 7.13 (d, J=7.0 Hz, 1H), 6.71 (m, 1H), 6.50 (d, J=6.3 Hz, 2H), 5.18 (m, 1H), 4.46 (d, J=7.8 Hz, 1H), 3.24 (s, 3H), 2.89 (dd, J=13.2, 7.8 Hz, 1H), 2.74 (dd, J=13.4, 6.8 Hz, 1H), 2.61 (s, 3H), 1.41 (s, 9H).

### Intermediate 102

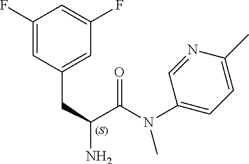

To a solution of Intermediate 101 (0.67 g, 1.7 mmol) in dioxane (6 mL) was added HCl (4N in dioxane) (1.50 mL, 6 mmol) and the reaction mixture was stirred at rt for 17 h. Methanol (2 mL) was added, the mixture was sonicated to get a clear solution, and then the reaction mixture was stirred for 8 h. The solvent was evaporated and dried under high vacuum to afford the HCl salt of the title compound (0.62 g) as off-white solid. LC-MS retention time=2.26 min; m/z=306.06 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Intermediate 103

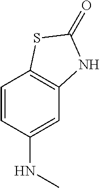

To a solution of 5-aminobenzo[d]thiazol-2(3H)-one (300 mg, 1.81 mmol) in methanol (10 mL) was added formaldehyde (108 mg, 3.61 mmol) and sodium methanolate (2.1 mL, 9.0 mmol) and the reaction mixture was heated to 60° C. for 16 h. It was cooled to rt and sodium tetrahydroborate (171 mg, 4.51 mmol) was added and the reaction mixture was stirred at rt for 30 min. Then methanol (2 mL) was added to the reaction mixture and it was stirred at rt for 6 h. Silica gel was added to the reaction mixture, the solvent was evaporated, and the residue was dry loaded onto a Biotage Horizon (0-100% EtOAc/Hexane, 20% MeOH/EtOAc and 100% MeOH) for purification. The material collected was further purified by preparative HPLC to afford the title compound (140 mg) as off-white. LC-MS retention time=1.36 min; m/z=181.00 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 11.56 (s, 1H), 7.24 (d, J=8.6 Hz, 1H), 6.47 (d, J=8.5 Hz, 1H), 6.40 (s, 1H), 2.70 (s, 3H).

### Intermediate 104

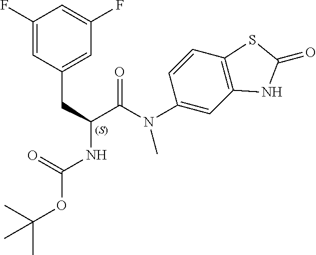

To a solution of (S)-2-((tert-butoxycarbonyl)amino)-3-(3,5-difluorophenyl)propanoic acid (143 mg, 0.48 mmol) and Intermediate 103 2,2,2-trifluoroacetate (140 mg, 0.48 mmol) in DMF (4 mL) was added DIPEA (0.20 mL, 1.2 mmol) and then HATU (190 mg, 0.50 mmol) and the reaction mixture was stirred at rt for 16 h. The reaction mixture was partitioned between EtOAc (20 mL) and water (40 mL) and the organic component was dried with Na₂SO₄, filtered concentrated and purified with a Biotage Horizon (10-100% EtOAc/Hexane) to afford the title compound (140 mg) as white foam. LC-MS retention time=3.83 min; m/z=486.02 [M+Na]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 12.07 (br. s, 1H), 7.69 (d, J=7.8 Hz, 1H), 7.13-7.00 (m, 4H), 6.50 (s, 1H), 4.19 (br. S, 1H), 3.18 (s, 3H), 2.84 (d, J=12.5 Hz, 1H), 2.73 (d, J=10.3 Hz, 1H), 1.28 (s, 9H).

### Intermediate 105

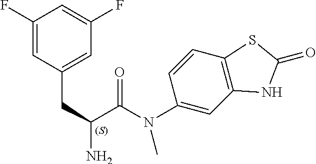

To a solution of Intermediate 104 (140 mg, 0.30 mmol) in dioxane (1 mL) was added HCl (4N in dioxane) (0.46 mL, 1.8 mmol) and the reaction mixture was stirred at rt for 16 h. Methanol (1 mL) was added and the stirring was continued for 8 h. The solvent was evaporated and the residue was dried under high vacuum to afford an HCl salt of the title compound (0.13 g) as light yellow solid. LC-MS retention time=2.91 min; m/z=364.02 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Intermediate 106

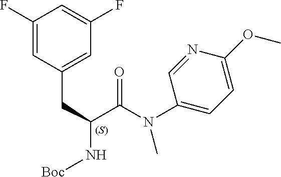

To a solution of (S)-2-((tert-butoxycarbonyl)amino)-3-(3,5-difluorophenyl)propanoic acid (0.57 g, 1.88 mmol) and 6-methoxy-N-methylpyridin-3-amine (0.26 g, 1.9 mmol) in DMF (7 mL) was added DIPEA (0.66 mL, 3.8 mmol) and then HATU (0.75 g, 2.0 mmol) and the reaction mixture was stirred at rt for 2 h. The reaction mixture was partitioned between water (70 mL) and EtOAc (35 mL), the organic component was separated and dried with Na₂SO₄, the solvent was evaporated to afford a crude oil which was purified with a Biotage Horizon (0-100% EtOAc) to afford the title compound (0.73 g) as a pink foam. LC-MS retention time=3.93 min; m/z=444.10 [M+Na]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 7.73 (s, 1H), 7.09 (s, 1H), 6.71 (d, J=8.8 Hz, 1H), 6.65 (m, 1H), 6.49 (d, J=6.0 Hz, 2H), 5.33 (m, 1H), 4.43 (m, 1H), 3.92 (s, 3H), 3.17 (s, 3H), 2.87 (dd, J=13.3, 7.5 Hz, 1H), 2.72 (dd, J=13.3, 7.5 Hz, 1H), 1.36 (s, 9H)

### Intermediate 107

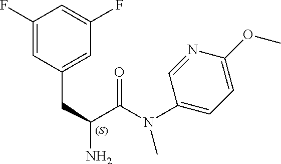

To a solution of Intermediate 106 (0.73 g, 1.7 mmol) in dioxane (6 mL) was added HCl (4N in dioxane) (1.6 mL, 6.4 mmol) and the reaction mixture was stirred at rt for 18 h. Methanol (2 mL) was added, the reaction was sonicated to get a clear solution and the reaction mixture was stirred at rt for 5 h. The solvent was evaporated and the residue was dried under high vacuum overnight to afford the title compound (0.68 g) as a brown solid. LC-MS retention time=2.92 min; m/z=322.08 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Intermediate 108

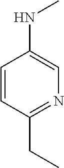

To a solution of 6-ethylpyridin-3-amine (500 mg, 4.09 mmol) and formaldehyde (184 mg, 6.14 mmol) in methanol (15 mL) was added sodium methanolate (4.7 mL, 20 mmol) and the reaction mixture was heated at 50° C. for 16 h. The reaction mixture was cooled to rt and sodium tetrahydroborate (387 mg, 10.2 mmol) was added in two portions. The reaction mixture was stirred at rt for 2 h, slowly diluted with water (10 mL) and then extracted by EtOAc (2×20 mL). The combined organic components were dried with Na₂SO₄, filtered, concentrated and purified with a Biotage Horizon (20-70% EtOAc/Hexane) to afford the title compound (0.41 g) as brown oil. LC-MS retention time=2.30 min; m/z=137.05 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, CDCl₃-d) δ 7.99 (s, 1H), 7.00 (d, J=8.3 Hz, 1H), 6.87 (d, J=11.2 Hz, 1H), 3.65 (br. s, 1H), 2.87 (s, 3H), 2.74 (q, J=7.6 Hz, 2H), 1.29 (t, J=7.6 Hz, 3H).

### Intermediate 109

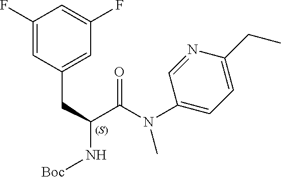

To a solution of (S)-2-((tert-butoxycarbonyl)amino)-3-(3,5-difluorophenyl)propanoic acid (0.55 g, 1.8 mmol) and Intermediate 108 (0.25 g, 1.8 mmol) in DMF (7 mL) was added DIPEA (0.64 mL, 3.7 mmol) and then HATU (0.73 g, 1.93 mmol) and the reaction mixture was stirred at rt for 16 h. The reaction mixture was partitioned between water (70 mL) and EtOAc (35 mL), the organic component was dried with Na₂SO₄, filtered, concentrated and purified with a Biotage Horizon (0-100% EtOAc/Hexane) to afford the title compound (0.42 g) as white oil. LC-MS retention time=3.54 min; m/z=442.09 [M+Na]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, CDCl₃-d) δ 8.16 (s, 1H), 7.20 (s, 2H), 6.70 (t, J=8.8 Hz, 1H), 6.47 (d, J=6.1 Hz, 2H), 5.22 (m, 1H), 4.47 (d, J=7.3 Hz, 1H), 3.25 (s, 3H), 2.90 (m, 3H), 2.72 (q, J=7.6 Hz, 1H), 1.41 (s, 9H), 1.35 (t, J=7.6 Hz, 3H).

### Intermediate 110

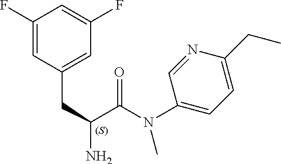

To a solution of Intermediate 109 (0.42 g, 1.0 mmol) in dioxane (1 mL) was added HCl (1.1 mL, 4.4 mmol, 4N in dioxane) and the cloudy solution was stirred at rt for 4 h. Methanol (1 mL) was added and the stirring was continued at rt for 16 h. The reaction mixture was concentrated to afford an HCl salt of the title compound (0.26 g). LC-MS retention time=2.37 min; m/z=320.12 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Intermediate 111

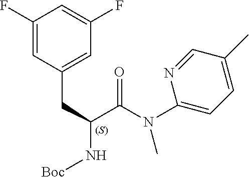

To a solution of (S)-2-((tert-butoxycarbonyl)amino)-3-(3,5-difluorophenyl)propanoic acid (0.617 g, 2.05 mmol) and N,5-dimethylpyridin-2-amine (0.25 g, 2.1 mmol) in DMF (7 mL) was added DIPEA (0.7 mL, 4 mmol) and then HATU (0.817 g, 2.15 mmol) and the reaction mixture was stirred at rt for 2 h. The reaction mixture was partitioned between water (70 mL) and EtOAc (35 mL). The organic component was dried with Na₂SO₄, filtered, concentrated and purified with a Biotage Horizon (0-100% EtOAc/Hexane) to afford the title compound (0.29 g) as light yellow oil. LC-MS retention time=3.88 min; m/z=406.12 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, CDCl₃-d) δ 8.36 (s, 1H), 7.61 (d, J=9.5 Hz, 1H), 7.05 (m, 1H), 6.65 (t, J=9.0 Hz, 1H), 6.54 (br. s, 1H), 5.32 (d, J=8.5 Hz, 1H), 4.75 (br. s, 1H), 3.34 (s, 3H), 3.06 (dd, J=13.5, 5.2 Hz, 1H), 2.99 (br. s, 1H), 2.40 (s, 3H), 1.44-1.39 (two s, 9H)

### Intermediate 112

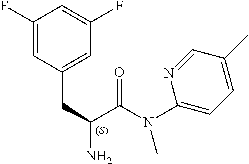

To a solution of Intermediate 111 (0.29 g, 0.72 mmol) in dioxane (2 mL) was added HCl (4N in dioxane) (1.1 mL, 4.4 mmol) and the reaction mixture was stirred at rt for 20 h. The solvent was evaporated and dried under high vacuum for 64 h to afford an HCl salt of the title compound (0.19 g) as pink solid. LC-MS retention time=2.75 min; m/z=328.04 [M+Na]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Intermediate 113

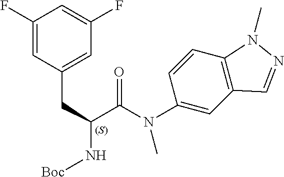

To a solution of (S)-2-((tert-butoxycarbonyl)amino)-3-(3,5-difluorophenyl)propanoic acid (0.82 g, 2.73 mmol) and N,1-dimethyl-1H-indazol-5-amine (0.44 g, 2.73 mmol) in DMF (7 mL) was added DIPEA (0.95 mL, 5.46 mmol) and then HATU (1.09 g, 2.87 mmol) and the reaction mixture was stirred at rt for 19 h. The reaction mixture was partitioned between water (70 mL) and EtOAc (35 mL), the organic component was dried with Na₂SO₄, filtered, concentrated and purified with a Biotage Horizon to afford the title compound (1.14 g) as pink foam. LC-MS retention time=3.69 min; m/z=467.07 [M+Na]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 8.10 (s, 1H), 7.77 (d, J=8.8 Hz, 2H), 7.71 (s, 1H), 7.08 (d, J=8.0 Hz, 1H), 6.95 (t, J=9.6 Hz, 1H), 6.65-6.39 (m, 2H), 4.09 (m overlapped with s, 4H), 3.21 (s, 3H), 2.87 (d, J=10.5 Hz, 1H), 2.70 (t, J=9.3 Hz, 1H), 1.28 (two s, 9H).

### Intermediate 114

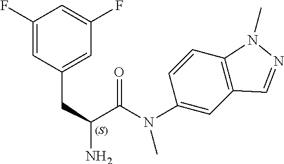

To a solution of Intermediate 113 (1.14 g, 2.56 mmol) in dioxane (4 mL) was added HCl (4N in dioxane) (2.4 mL, 9.6 mmol) and the reaction mixture was stirred at rt for 1 h. Precipitate formed and methanol (1 mL) was added to reform a homgeneous solution and the stirring was continued for 24 h. The solvent was removed and the residue was dried under high vacuum to afford an HCl salt of the title compound (1.03 g) as light brown solid. LC-MS retention time=2.67 min; m/z=345.10 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Intermediate 115

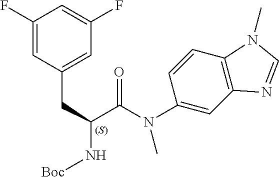

To a solution of (S)-2-((tert-butoxycarbonyl)amino)-3-(3,5-difluorophenyl)propanoic acid (1278 mg, 4.24 mmol) and N,1-dimethyl-1H-benzo[d]imidazol-5-amine (570 mg, 3.54 mmol) in DCM (20 mL) was added N-ethoxycarbonyl-2-ethoxy-1,2-dihydroquinoline (1.05 g, 4.24 mmol) and the reaction mixture was stirred at rt for 17 h. The solvent was evaporated and the residue was purified with a Biotage Horizon (30-100% Hexane/EtOAc, 10-20% MeOH/EtOAc) to afford the title compound (0.13 g) as pink solid. LC-MS retention time=3.29 min; m/z=467.08 [M+Na]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 8.32 (s, 1H), 7.71 (d, J=8.2 Hz, 2H), 7.28 (d, J=7.3 Hz, 1H), 7.12 (d, J=8.3 Hz, 1H), 6.95 (t, J=9.2 Hz, 1H), 6.34 (d, J=7.0 Hz, 2H), 4.25-4.14 (m, 1H), 3.90 (s, 3H), 3.23 (s, 3H), 2.86 (d, J=13.1 Hz, 1H), 2.68 (t, J=10.3 Hz, 1H), 1.28 (s, 9H).

### Intermediate 116

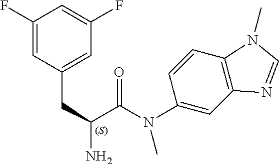

To a solution of Intermediate 115 (0.13 g, 0.29 mmol) in dioxane (2 mL) was added HCl (4N in dioxane) (0.9 mL, 3.6 mmol) and the reaction mixture was stirred at rt for 4 h, treated with methanol (0.5 mL) and stirred for an additional 4 h. The solvent was evaporated and the residue was dried under high vacuum to afford an HCl salt of the title compound (0.12 g) as light yellow solid. LC-MS retention time=1.97 min; m/z=345.19 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Intermediate 117

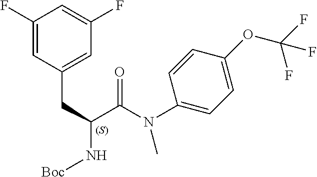

To a solution of (S)-2-((tert-butoxycarbonyl)amino)-3-(3,5-difluorophenyl)propanoic acid (0.79 g, 2.6 mmol) in DMF (6 mL) was added N-methyl-4-(trifluoromethoxy)aniline (0.500 g, 2.62 mmol), DIPEA (0.91 mL, 5.2 mmol) and HATU (1.04 g, 2.75 mmol) and the reaction mixture was stirred at rt for 2 h. The reaction mixture was partitioned between water (60 mL) and EtOAc (30 mL). The organic component was dried with Na₂SO₄, filtered, concentrated and purified with a Biotage Horizon (0-100% EtOAc/Hexane) to afford the title compound (0.80 g) as pink solid. LC-MS retention time=4.25 min; m/z=375.14 [M-Boc+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 7.55 (s, 4H), 7.25 (d, J=7.7 Hz, 1H), 7.02 (m, 1H), 6.42 (m, 2H), 4.10 (m, 1H), 3.19 (s, 3H), 2.80-2.65 (m, 2H), 1.30 (s, 9H).

### Intermediate 118

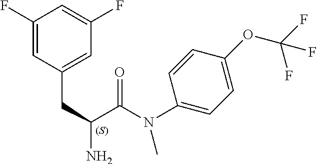

To a solution of Intermediate 117 (0.800 g, 1.68 mmol) in dioxane (3 mL) was added HCl (4N in dioxane) (3.1 mL, 12.4 mmol) and the reaction mixture was stirred at rt for 17 h. The reaction mixture was concentrated and dried under high vacuum to afford an HCl salt of the title compound (0.65 g) as white solid . LC-MS retention time=3.31 min; m/z=375.15 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Intermediate 119

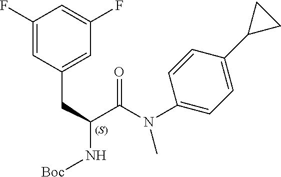

To a solution of (S)-2-((tert-butoxycarbonyl)amino)-3-(3,5-difluorophenyl)propanoic acid (1.02 g, 3.4 mmol) and 4-cyclopropyl-N-methylaniline (0.5 g, 3.4 mmol) in DMF (7 mL) was added DIPEA (1.2 mL, 6.8 mmol) and then HATU (1.36 g, 3.6 mmol) and the reaction mixture was stirred at rt for 24 h. The reaction mixture was partitioned between EtOAc (35 mL) and water (70 mL), the organic component was separated, dried with Na₂SO₄, concentrated and purified with a Biotage Horizon (0-50% EtOAc/hexane) to afford the title compound (1.03 g) as light brown solid. LC-MS retention time=4.24 min; m/z=453.20 [M+Na]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 7.26 (m, 4H), 7.14 (d, J=8.6 Hz, 1H), 7.00 (t, J=9.3 Hz, 1H), 6.35 (m, 2H), 4.23-4.11 (m, 1H), 3.16 (two s, 3H), 2.75-2.60 (m, 2H), 2.00 (m, 1H), 1.30 (s, 7.5H), 1.10 (s, 1.5H), 1.00 (m, 2H), 0.72 (m, 2H).

### Intermediate 120

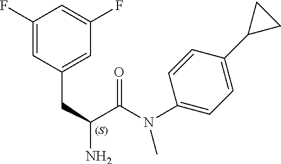

To a solution of Intermediate 119 (1.03 g, 2.39 mmol) in dioxane (5 mL) was added HCl (4N in dioxane) (3.63 mL, 14.4 mmol) and the reaction mixture was stirred at rt for 5 h. Methanol (5 drops) was added and the stirring continued at rt for 39 h. The reaction mixture was concentrated and dried under high vacuum overnight to afford an HCl salt of title compound (0.87 g) as brown solid. LC-MS retention time=3.38 min; m/z=331.17 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Intermediate 125

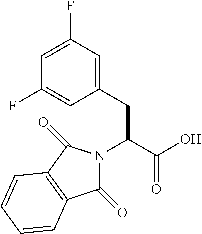 [SMILES: O=C(O)[C@H](Cc1cc(F)cc(F)c1)N1C(=O)c2ccccc2C1=O]

A mixture of (S)-2-amino-3-(3,5-difluorophenyl)propanoic acid (1.72 g, 8.55 mmol) and isobenzofuran-1,3-dione (1.27 g, 8.55 mmol) in DMF (12 mL) in a microwave vial (20 mL) was heated at 155° C. for 1 h in microwave reactor. The crude mixture was poured into warm water (50 mL) and stirred for 30 min. The solids were collected by filtration, washed with water and dried under high vacuum overnight to afford the title compound (2.3 g) as light brown solid. LC-MS retention time=3.57 min; m/z=354.07 [M+Na]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 13.45 (br. s, 1H), 7.87 (s, 4H), 7.02-6.94 (m, 3H), 5.23 (d, J=4.8 Hz, 0.5H), 5.20 (d, J=4.8 Hz, 0.5H), 3.53 (d, J=4.5 Hz, 0.5H), 3.50 (d, J=4.5 Hz, 0.5 H), 3.35 (m, 1H, overlapped with water peak).

### Intermediate 126

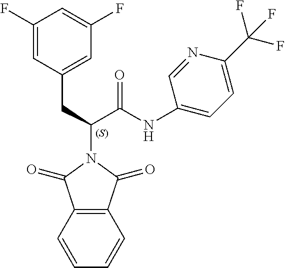

The solution of Intermediate 125 (500 mg, 1.51 mmol) in SOCl₂ (1.1 mL, 15 mmol) was heated at 45° C. for 2.5 h. The reaction mixture was concentrated, azeotroped with DCM (3×10 mL), dried under high vacuum overnight to afford the (S)-3-(3,5-difluorophenyl)-2-(1,3-dioxoisoindolin-2-yl)propanoyl chloride (0.49 g) as white solid. To a solution of (S)-3-(3,5-difluorophenyl)-2-(1,3-dioxoisoindolin-2-yl)propanoyl chloride (440 mg, 1.26 mmol) and 6-(trifluoromethyl)pyridin-3-amine (204 mg, 1.26 mmol) in DMF (6 mL) was added DIPEA (0.44 mL, 2.52 mmol) and the reaction mixture was stirred at rt for 2 h. The reaction mixture was partitioned between water (60 mL) and EtOAc (30 mL), the organic component was separated and dried with Na₂SO₄, filtered, concentrated and purified with a Biotage Horizon (10-100% EtOAc/Hexane) to afford the title compound (0.39 g) as white solid. LC-MS retention time=3.92 min; m/z=476.11 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 10.50 (s, 1H), 8.86 (s, 1H), 8.30 (d, J=10.8 Hz, 1H), 7.88 (m, 5H), 7.01 (m, 1H), 6.94 (d, J=8.5 Hz, 2H), 5.35 (d, J=4.8 Hz, 0.5H), 5.33 (d, J=4.8 Hz, 0.5H), 3.65 (d, J=4.5 Hz, 0.5H), 3.62 (d, J=4.5 Hz, 0.5 H), 3.29 (m, 1H).

### Intermediate 127

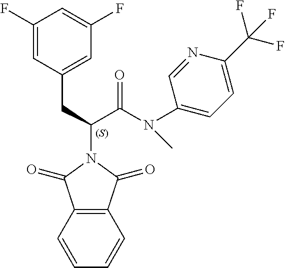

To a solution of Intermediate 126 (0.39 g, 0.8 mmol) in DMF (6 mL) was added 60% NaH (0.036 g, 0.9 mmol) as a dispersion in mineral oil and the reaction mixture was stirred for 5 min. Then iodomethane (0.06 mL, 0.9 mmol) was added and the reaction mixture was stirred at rt for 23 h. The reaction mixture was diluted with sat. aq. NH₄Cl (5 mL) and water (5 mL) and then extracted with EtOAc (2×20 mL), the combined organic component was dried with Na₂SO₄, filtered, concentrated and purified with a Biotage (0-80% EtOAc/Hexane) to afford the title compound (0.19 g) as colorless oil. LC-MS retention time=3.73 min; m/z=490.13 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 8.61 (s, 1H), 7.96 (d, J=8.8 Hz, 1H), 7.80 (m, 2H), 7.66 (br. s, 3H), 6.96 (t, J=9.6 Hz, 1H), 6.83 (d, J=6.7 Hz, 2H), 5.30 (br. s, 1H), 3.47 (d, J=4.5 Hz, 0.5H), 3.43 (d, J=4.5 Hz, 0.5 H), 3.19 (s, 3H), 3.16 (t, J=11.3 Hz, 1H).

### Intermediate 128

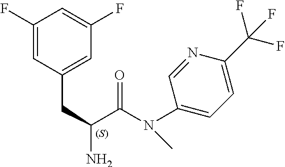

To a solution of Intermediate 127 (0.19 g, 0.4 mmol) in ethanol (6 mL) was added hydrazine hydrate (0.1 mL, 2.3 mmol) and the reaction mixture was heated at 50° C. for 5 h. The cooled reaction mixture was filtered and the filtrate was concentrated, azeotroped with ethanol (2×10 mL) and dried under high vacuum for 72 h to afford the title compound (0.14 g) as sticky solid. LC-MS retention time=2.87 min; m/z=360.11 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Intermediate 129

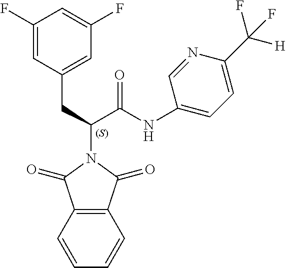

To a solution of (S)-3-(3,5-difluorophenyl)-2-(1,3-dioxoisoindolin-2-yl)propanoyl chloride (1.21 g, 3.47 mmol) and 6-(difluoromethyl)pyridin-3-amine (0.50 g, 3.5 mmol) in DMF (6 mL) was added DIPEA (1.2 mL, 6.94 mmol) and the reaction mixture was stirred at rt for 2 h. The reaction mixture was partitioned between water (60 mL) and EtOAc (30 mL), the organic component was dried with Na₂SO₄, filtered, concentrated and purified with a Biotage Horizon (10-100% EtOAc/Hexane) to afford the title compound (0.9 g) as white solid. LC-MS retention time=3.79 min; m/z=458.13 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 10.37 (s, 1H), 8.80 (s, 1H), 8.20 (d, J=8.5 Hz, 1H), 7.88 (s, 4H), 7.70 (d, J=8.8 Hz, 1H), 7.06-6.92 (m, 4H), 5.32 (dd, J=13.8, 4.5 Hz, 1H), 3.63 (dd, J=13.8, 4.5 Hz, 1H), 3.30 (m, 1H).

### Intermediate 130

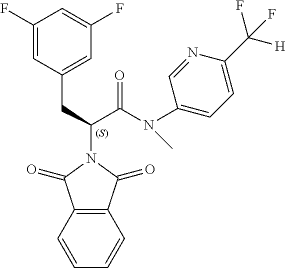

To a solution of Intermediate 129 (0.90 g, 2.0 mmol) in DMF (20 mL) was added 60% NaH (0.087 g, 2.2 mmol) as a dispersion in mineral oil and the reaction mixture was stirred for 5 min. Then iodomethane (0.14 mL, 2.2 mmol) was added and the stirring was continued at rt for 23 h. Sat. aq. NH₄Cl (10 mL) was added slowly, followed by water (100 mL), and the reaction mixture was extracted by EtOAc (2×20 mL). The combined organic component was dried with Na₂SO₄, filtered, concentrated and purified with a Biotage Horizon (0-60% EtOAc/Hexane, 60-100% EtOAc/Hexane) to afford the title compound (0.7 g) as white solid. LC-MS retention time=3.58 min; m/z=494.11 [M+Na]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Intermediate 131

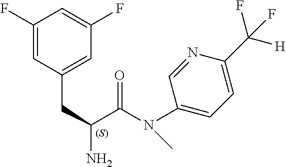

To a solution of Intermediate 130 (0.44 g, 0.93 mmol) in ethanol (10 mL) was added hydrazine hydrate (0.27 mL, 5.60 mmol) and the reaction mixture was heated at 50° C. for 5 h. The reaction mixture was filtered and the filtrate was concentrated, azeotroped with ethanol (2×10 mL) and dried under high vacuum for 64 h to afford the title compound (0.29 g) as white solid. LC-MS retention time=2.56 min; m/z=364.13 [M+Na]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Intermediate 132

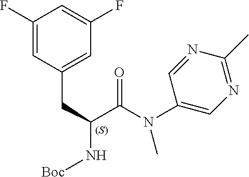

To a solution of (S)-2-((tert-butoxycarbonyl)amino)-3-(3,5-difluorophenyl)propanoic acid (1003 mg, 3.33 mmol) and N,2-dimethylpyrimidin-5-amine (410 mg, 3.33 mmol) in DMF (5 mL) was added DIPEA (1.2 mL, 6.7 mmol) and HATU (1329 mg, 3.50 mmol) and the reaction mixture was stirred at rt for 4 h. The reaction mixture was partitioned between water (50 mL) and EtOAc (25 mL), the organic component was dried with Na₂SO₄, filtered, concentrated and purified with a Biotage Horizon (70-100% EtOAc/Hexane) to afford the title compound (0.6 g) as white solid. LC-MS retention time=3.60 min; m/z=429.17 [M+Na]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 8.77-8.61 (two s, 2H), 7.33 (m, 1H), 7.05-6.69 (m, 3H), 4.12 (m, 1H), 3.17 (s, 3H), 2.89 (m, 1H), 2.75 (m, 1H), 2.62 (s, 3H), 1.24 (s, 9H).

### Intermediate 133

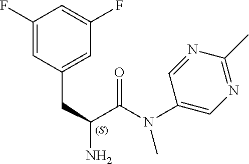

To a solution of Intermediate 132 (0.60 g, 1.5 mmol) in dioxane (5 mL) was added HCl (4N in dioxane) (2.2 mL, 8.8 mmol) and the reaction mixture was stirred at rt for 19 h. The reaction mixture was concentrated and dried under high vacuum overnight to afford an HCl salt of title compound (0.55 g) as light brown solid. LC-MS retention time=2.29 min; m/z=307.15 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Intermediate 140

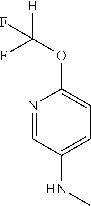

To a solution of 6-(difluoromethoxy)pyridin-3-amine (250 mg, 1.56 mmol) and formaldehyde (70 mg, 2.3 mmol) in methanol (8 mL) was added sodium methanolate (1.8 mL, 7.8 mmol) and the reaction mixture was heated at 50° C. for 18 h. The reaction mixture was cooled to rt and sodium tetrahydroborate (148 mg, 3.90 mmol) was added in two portions and the mixture was stirred at rt for 3 h. Water (5 mL) was added slowly and the reaction mixture was extracted with EtOAc (2×20 mL). The combined organic component was dried with Na₂SO₄, filtered, concentrated and purified with a Biotage Horizon (20-100% EtOAc/Hexane) to afford the title compound (0.23 g) as colorless oil. LC-MS retention time=1.94 min; m/z=175.05 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 7.51 (d, J=2.7 Hz, 1H), 7.46 (t, J=74.1 Hz, 1H), 7.10 (dd, J=8.8, 3.1 Hz, 1H), 6.85 (d, J=8.8 Hz, 1H), 5.81 (m, 1H), 2.69 (d, J=5.2 Hz, 3H).

### Intermediate 141

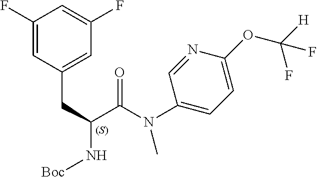

To a solution of (S)-2-((tert-butoxycarbonyl)amino)-3-(3,5-difluorophenyl)propanoic acid (398 mg, 1.32 mmol) and Intermediate 140 (0.23 g, 1.3 mmol) in DMF (5 mL) was added DIPEA (0.50 mL, 2.6 mmol) and then HATU (527 mg, 1.39 mmol) and the reaction mixture was stirred at rt for 18 h. The reaction mixture was partitioned between water and EtOAc. The organic component was dried with Na₂SO₄, filtered, concentrated to afford the title compound (0.48 g) as a light yellow solid. LC-MS retention time=4.00 min; m/z=480.13 [M+Na]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 8.36-8.27 (two s, 1H), 7.92 (m, 1H), 7.74 (t, J=72.5 Hz, 1H), 7.27-7.21-7.04 (m, 3H), 6.63 (m, 2H), 4.11 (m, 1H), 3.16 (s, 3H), 2.86 (d, J=3.6 Hz, 1H), 2.73 (d, J=3.6 Hz, 1H), 1.26 (s, 9H).

### Intermediate 142

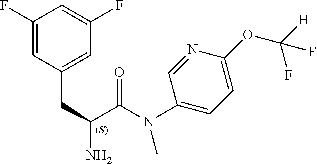

To a solution of Intermediate 141 (0.48 g, 1.1 mmol) in dioxane (4 mL) was added HCl (4N in dioxane) (1.6 mL, 6.4 mmol) and the reaction mixture was stirred at rt for 5 h. The reaction mixture was concentrated and dried under high vacuum overnight to afford an HCl salt of title compound (0.51 g) as glassy brown solid. LC-MS retention time=2.92 min; m/z=358.14 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Intermediate 143

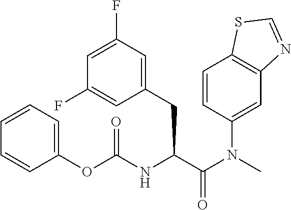 [SMILES: CN(C(=O)[C@H](Cc1cc(F)cc(F)c1)NC(=O)Oc1ccccc1)c1ccc2scnc2c1]

To a solution of Intermediate 20 (0.517 g, 1.35 mmol) in THF (30 mL) was added phenyl carbonochloridate (0.17 mL, 1.4 mmol) followed by the addition of triethylamine (0.563 mL, 4.04 mmol). The reaction mixture was stirred at rt under nitrogen for 4 h. All solvents were removed in vacuo. The residue was taken up in CH₂Cl₂ (6 mL) and loaded on an ISCO silica gel cartridge (120 g) eluting with 40% EtOAc/hexanes to afford the title compound (542.9 mg) as a white foam. LC-MS retention time=3.78 min; m/z=468.06 [M+H]⁺, 490.06 [M+Na]⁺. (Column: Phenomenex Luna C18 50×2.0 mm 3 μm. Solvent A=90% Water: 10% MeOH: 0.1% TFA. Solvent B=10% Water: 90% MeOH: 0.1% TFA. Flow Rate=0.8 mL/min. Start % B=0. Final % B=100. Gradient Time=4 minutes, then a 1-minute hold at 100% B. Oven temperature=40° C. Wavelength=220 nm).

### Intermediate 144

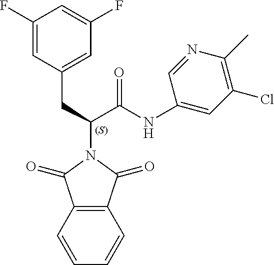

To a solution of (S)-3-(3,5-difluorophenyl)-2-(1,3-dioxoisoindolin-2-yl)propanoyl chloride (1079 mg, 3.09 mmol) and 5-chloro-6-methylpyridin-3-amine (440 mg, 3.09 mmol) in DMF (5 mL) was added DIPEA (1.1 mL, 6.17 mmol) and the reaction mixture was stirred at rt for 16 h. The reaction mixture was partitioned between water (50 mL) and EtOAc (25 mL) and the organic component was dried with Na₂SO₄, concentrated and purified with a Biotage Horizon (10-100% EtOAc/Hexane) to afford the title compound (0.66 g) as white solid. LC-MS retention time=3.64 min; m/z=456.11 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 10.22 (s, 1H), 8.54 (d, J=2.3 Hz, 1H), 8.11 (d, J=2.3 Hz, 1H), 7.87 (m, 4H), 7.00 (m, 1H), 6.92 (d, J=6.3 Hz, 2H), 5.30 (m, 1H), 3.61 (dd, J=13.8, 4.5 Hz, 1H), 3.28 (m, 1H), 2.51 (s, 3H, overlapped with DMSO).

### Intermediate 145

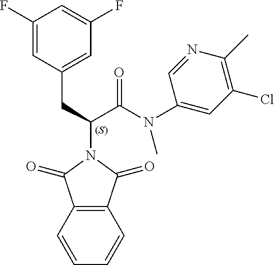

To a solution of Intermediate 144 (0.66 g, 1.5 mmol) in DMF (10 mL) was added 60% NaH (0.064 g, 1.6 mmol) as a dispersion in mineral oil and the reaction mixture was stirred at rt for 5 min. Then iodomethane (0.1 mL, 1.59 mmol) was added and the reaction mixture was stirred at rt for 17 h and then heated to 60° C. for 1 h. To the cooled reaction mixture, Sat. NH₄Cl (5 mL) was added slowly and followed by water (100 mL), it was extracted by EtOAc (2×20 mL), the combined organic component was dried with Na₂SO₄, concentrated and purified with a Biotage Horizon (0-70% EtOAc/Hexane) to afford the title compound (0.14 g) as white solid. LC-MS retention time=3.80 min; m/z=470.07 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Intermediate 146

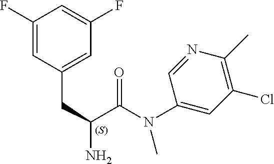

To a solution of Intermediate 145 (0.16 g, 0.34 mmol) in ethanol (5 mL) was added hydrazine hydrate (0.10 mL, 2.0 mmol) and the reaction mixture was heated at 50° C. for 5 h. The reaction mixture was filtered and the filtrate was concentrated, azeotroped with ethanol (2×10 mL) and dried under high vacuum overnight to afford the title compound as white solid (90 mg). LC-MS retention time=2.95 min; m/z=340.09 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Intermediate 147

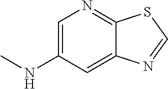

To a solution of thiazolo[5,4-b]pyridin-6-amine (0.50 g, 3.3 mmol) and formaldehyde (0.149 g, 4.96 mmol) in MeOH (20 mL) was added sodium methanolate (3.78 mL, 16.5 mmol) and the reaction mixture was heated to 50° C. for 18 h. The reaction mixture was cooled to rt, treated sodium tetrahydroborate (0.313 g, 8.27 mmol) in two portions and the reaction mixture was stirred at rt for 2 h. Water (5 mL) was added slowly and most of solvent was removed. The resulting mixture was extracted with EtOAc (20 mL), the organic component was dried with Na₂SO₄, filtered, concentrated and purified by Biotage Horizon (20-100% EtOAc/Hexane) to afford the title compound (0.36 g) as pink solid. LC-MS retention time=1.04 min; m/z=166.03 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 9.36 (s, 1H), 8.15 (d, J=2.5 Hz, 1H), 7.41 (d, J=2.5 Hz, 1H), 6.25 (m, 1H), 2.78 (d, J=5.0 Hz, 3H)

### Intermediate 148

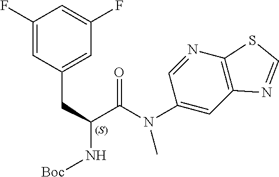

To a solution of (S)-2-((tert-butoxycarbonyl)amino)-3-(3,5-difluorophenyl)propanoic acid (656 mg, 2.18 mmol) in DMF (2 mL) was added Intermediate 147 (360 mg, 2.18 mmol), DIPEA (0.76 mL, 4.4 mmol) and HATU (870 mg, 2.29 mmol) and the reaction mixture was stirred at rt for 16 h. The reaction mixture was partitioned between water (20 mL) and EtOAc (10 mL). The organic component was dried with Na₂SO₄, filtered, concentrated and purified with a Biotage Horizon (0-80% EtOAc/Hexane) to afford the title compound (0.54 g) as light yellow foam. LC-MS retention time=3.71 min; m/z=471.11 [M+Na]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 9.63 (s, 1H), 8.61 (s, 1H), 8.45 (s, 1H), 7.28-6.99 (m, 2H), 6.58 (m, 2H), 4.15 (m, 1H), 3.26 (two s, 3H), 2.93 (m, 1H), 2.76 (m, 1H), 1.18 (s, 9H).

### Intermediate 149

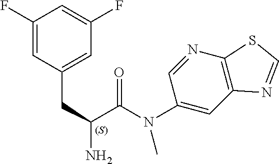

To a solution of Intermediate 148 (500 mg, 1.12 mmol) in dioxane (5 mL) was added HCl (4N in dioxane) (3.4 mL, 14 mmol) and methanol (5 drops) and the reaction mixture was stirred at rt for 5 h. The reaction mixture was concentrated and dried under high vacuum overnight to afford an HCl salt of title compound (0.42 g) as orange solid. LC-MS retention time=2.27 min; m/z=349.10 [M+H]⁺. (Column: Phenomenex-Luna C18 2.0×50 mm, 3 μm particles; Mobile Phase A: 5% ACN-95% H₂O-0.1% TFA; Mobile Phase B: 95% ACN-5% H₂O-0.1% TFA; Temperature: 40° C.; Gradient: 0-100% B over 4 min, then a 1-min hold at 100% B; Flow: 0.8 mL/min; Detection: UV at 220 nm).

### Intermediate 150

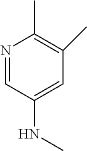

To a suspension of 5,6-dimethylpyridin-3-amine (650 mg, 5.32 mmol) and formaldehyde (240 mg, 7.98 mmol) in methanol (20 mL) was added sodium methanolate (6.08 mL, 26.6 mmol) and the reaction mixture was heated to 50° C. for 16 h. The reaction mixture was cooled to rt, treated sodium tetrahydroborate (503 mg, 13.3 mmol) in two portions and stirred at rt for 3 h. The reaction mixture was concentrated and dry-loaded with silica gel onto Biotage Horizon (20-100% EtOAc/Hexane then 20% MeOH/EtOAc) for purification. The title compound (0.40 g) was obtained as white solid. LC-MS retention time=1.44 min; m/z=137.13 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 7.63 (d, J=2.7 Hz, 1H), 6.68 (d, J=2.5 H, 1H), 5.50 (m, 1H), 2.66 (d, 5.0 Hz, 3H), 2.25 (s, 3H), 2.14 (s, 3H).

### Intermediate 151

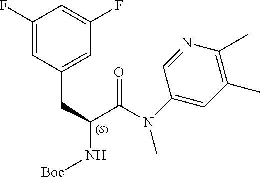

To a solution of (S)-2-((tert-butoxycarbonyl)amino)-3-(3,5-difluorophenyl)propanoic acid (0.885 g, 2.94 mmol) and Intermediate 150 (0.40 g, 2.9 mmol) in DMF (5 mL) was added DIPEA (1.02 mL, 5.87 mmol) and then HATU (1.17 g, 3.08 mmol) and the reaction mixture was stirred at rt for 4 h. The reaction mixture was partitioned between water (50 mL) and EtOAc (25 mL), the organic component was dried with Na₂SO₄, filtered, concentrated and purified with a Biotage Horizon (20-100% EtOAc/Hexane) to afford the title compound (0.80 g) as white solid. LC-MS retention time=3.27 min; m/z=442.20 [M+Na]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 8.32-8.25 (two s, 1H), 7.36 (s, 1H), 7.25 (d, J=8.0 Hz, 1H), 7.08 (m, 1H), 6.71-6.56 (m, 2H), 4.06 (m, 1H), 3.18-3.13 (two s, 3H), 2.83 (m, 1H), 2.72 (m, 1H), 2.45 (s, 3H), 2.26 (s, 3H), 1.29 (s, 9H).

### Intermediate 152

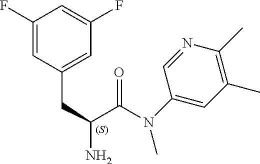

To a solution of Intermediate 151 (0.80 g, 1.9 mmol) in dioxane (6 mL) was added HCl (4N in dioxane) (2.90 mL, 11.6 mmol) and the reaction mixture was stirred at rt for 20 h. Most of the solvent was removed and HCl (4N in dioxane) (2.90 mL, 95 mmol) was added and the reaction mixture was stirred at rt for 2 h. Methanol (2 mL) was added and then reaction was stirred for 1 h, then additional methanol (2 mL) was added and the stirring was continued at rt for 20 h. The reaction mixture was concentrated and dried under high vacuum to afford an HCl salt of title compound (0.7 g) as white solid. LC-MS retention time=2.20 min; m/z=320.19 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Intermediate 153

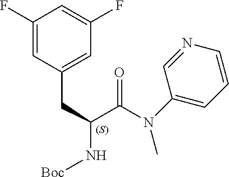

To a solution of (S)-2-((tert-butoxycarbonyl)amino)-3-(3,5-difluorophenyl)propanoic acid (0.697 g, 2.31 mmol) and N-methylpyridin-3-amine (0.25 g, 2.3 mmol) in DMF (5 mL) was added DIPEA (0.808 mL, 4.62 mmol) and then HATU (0.923 g, 2.427 mmol) and the reaction mixture was stirred at rt for 17 h. The reaction mixture was partitioned between water (50 mL) and EtOAc (25 mL). The organic component was dried with Na₂SO₄, concentrated and purified with a Biotage Horizon (20-100% EtOAc/Hexane) to afford the title compound (0.27 g) as white foam. LC-MS retention time=3.36 min; m/z=292.16 [M-Boc+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 8.58 (m, 2H), 7.80 (m, 1H), 7.54 (m, 1H), 7.30 (m, 1H), 7.04 (m, 2H), 6.53 (m, 1H), 4.15 (m, 1H), 3.20 (s, 3H), 2.84-2.72 (m, 2H), 1.29 (two s, 9H).

### Intermediate 154

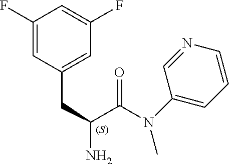

To a solution of Intermediate 153 (0.27 g, 0.69 mmol) in dioxane (2 mL) was added HCl (4N in dioxane) (1 mL, 4 mmol) and the reaction mixture was stirred at rt for 4 h. Methanol (1 mL) was added and the stirring was continued at rt for 16 h. The reaction mixture was concentrated and dried under high vacuum overnight to afford an HCl salt of title compound (0.23 g) as orange solid. LC-MS retention time=2.15 min; m/z=292.16 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Intermediate 155

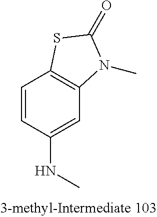

To a suspension of 5-amino-3-methylbenzo[d]thiazol-2(3H)-one (690 mg, 3.83 mmol) and formaldehyde (172 mg, 5.74 mmol) in methanol (20 mL) was added sodium methanolate (4.4 mL, 19 mmol) and the reaction mixture was heated to 50° C. for 16 h. The reaction mixture was cooled to rt, treated sodium tetrahydroborate (362 mg, 9.57 mmol) in two portions and then stirred at rt for 3 h. The reaction mixture was concentrated, the mixture was dry-loaded with silica gel to Biotage Horizon (0-80% EtOAc/Hexane) for purification to afford the title compound (0.54 g) as white solid. LC-MS retention time=1.84 min; m/z=195.11 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 8.50 (d, J=8.5 Hz, 1H), 6.45 (dd, J=8.5, 2.3, 1H), 6.40 (d, J=2.3 Hz, 1H), 5.91 (m, 1H), 3.34 (s, 3H, overlapped with water peak), 2.72 (d, J=5.0 Hz, 3H).

### Intermediate 156

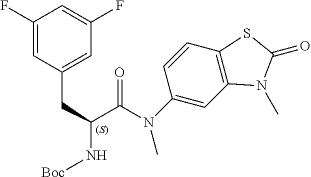

To a solution of (S)-2-((tert-butoxycarbonyl)amino)-3-(3,5-difluorophenyl)propanoic acid (0.392 g, 1.30 mmol) and 3-methyl-Intermediate 103 (0.23 g, 1.2 mmol) in DMF (5 mL) was added DIPEA (0.40 mL, 2.4 mmol) and HATU (0.495 g, 1.30 mmol) and the reaction mixture was stirred at rt for 3 h. The reaction mixture was partitioned between water (50 mL) and EtOAc (25 mL). The organic component was dried with Na₂SO₄, concentrated and purified twice with a Biotage Horizon (0-70% EtOAc/Hexane. 120 g column, then 10-70% EtOAc/Hexane) to afford the title compound (0.21 g) as white foam. LC-MS retention time=3.92 min; m/z=500.15 [M+Na]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 7.82-7.74 (m, 1H), 7.47-7.36 (two s, 1H), 7.22 (d, J=8.0 Hz, 1H), 7.09-7.00(m, 2H), 6.67-6.55 (m, 2H), 4.23 (m, 1H), 3.40 (s, 3H), 3.23-3.19 (two s, 3H), 2.89 (m, 1H), 2.71 (m, 1H), 1.28 (s, 9H).

### Intermediate 157

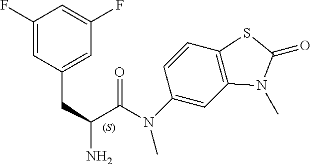

To a solution of Intermediate 156 (210 mg, 0.44 mmol) in dioxane (2 mL) was added HCl (4N in dioxane) (0.67 mL, 2.7 mmol) and the reaction mixture was stirred at rt for 24 h. The reaction mixture was concentrated and dried under high vacuum overnight to afford an HCl salt of title compound (0.19 g) as light pink solid. LC-MS retention time=2.67 min; m/z=378.10 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Intermediate 158

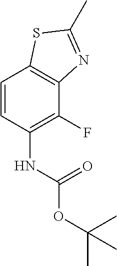 [SMILES: Cc1nc2c(F)c(NC(=O)OC(C)(C)C)ccc2s1]

To a solution of tert-butyl (2-methylbenzo[d]thiazol-5-yl)carbamate (1.28 g, 4.84 mmol) in acetonitrile (50 mL) was added selectfluor (3.43 g, 9.68 mmol) and the reaction mixture was stirred at rt for 19 h. The reaction mixture was partitioned between EtOAc (20 mL) and water (20 mL), the organic component was dried with Na₂SO₄, filtered and purified with a Biotage Horizon (0-40% EtOAc/hexane) to afford the title compound (0.33 g) as white solid. LC-MS retention time=3.86 min; m/z=283.13 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 9.06 (s, 1H), 7.76 (d, J=8.6 Hz, 1H), 7.54 (t, J=7.3 Hz, 1H), 2.82 (s, 3H), 1.48 (two s, 9H).

### Intermediate 159

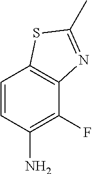

To a solution of Intermediate 158 (0.33 g, 1.2 mmol) in dioxane (2 mL) was added HCl (4N in dioxane) (1.1 mL, 4.4 mmol) and the reaction mixture was stirred at rt for 5 h, then methanol (1 mL) was added and the stirring was continued at rt for 7 h. Additional HCl (4N in dioxane) (1.06 mL, 4.24 mmol) was added and the stirring was continued at rt for 16 h. The reaction mixture was concentrated and dried under high vacuum to afford the title compound (0.29 g) as brown solid. LC-MS retention time=2.24 min; m/z=183.03 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Intermediate 160

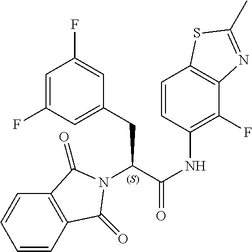

To a solution of Intermediate 125 (338 mg, 1.02 mmol) and Intermediate 159 (260 mg, 1.02 mmol) in DMF (1 mL) was added DIPEA (0.62 mL, 3.6 mmol) and HATU (407 mg, 1.09 mmol) and the reaction mixture was stirred at rt for 3 h. The reaction mixture was partitioned between water (10 mL) and EtOAc (5 mL), the organic component was dried with Na₂SO₄, filtered and purified with a Biotage Horizon (0-70% EtOAc/Hexane) to afford the title compound (0.28 g, with impurity). The material was purified again with a Biotage Horizon (10-70% EtOAc/Hexane, 120 g column) to afford the title compound (0.12 g) as green oil. LC-MS retention time=3.86 min; m/z=496.14 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) δ 10.08 (s, 1H), 7.86 (m, 5H), 7.45 (m, 1H), 6.94 (m, 3H), 5.33 (dd, J=9.0, 6.5 Hz, 1H), 3.64 (dd, J=13.8, 4.5 Hz, 1H), 3.40 (m, 1H), 2.83 (s, 3H).

### Intermediate 161

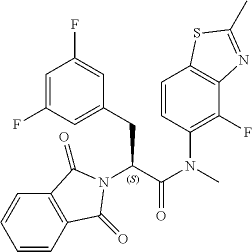

To a solution of Intermediate 160 (0.15 g, 0.30 mmol) in DMF (5 mL) was added 60% NaH (0.013 g, 0.33 mmol) as a dispersion in mineral oil and the reaction mixture was stirred at rt for 3 min, then iodomethane (0.02 mL, 0.33 mmol) was added and the stirring was continued at rt for 17 h. The reaction mixture was partitioned between water (50 mL) and EtOAc (25 mL), the organic component was dried with Na₂SO₄, filtered, concentrated and purified with a Biotage Horizon (0-80% EtOAc/Hexane) to afford the title compound (60 mg) as white solid. LC-MS retention time=3.92 min; m/z=510.12 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Intermediate 162

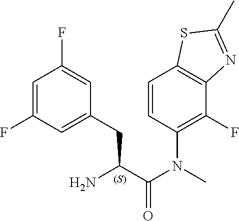

To a solution of Intermediate 161 (60 mg, 0.118 mmol) in ethanol (3 mL) was added hydrazine hydrate (0.03 mL, 0.71 mmol) and the reaction mixture was heated to 50° C. for 5 h. The solvent was concentrated, the residue was azeotroped by ethanol (2×10 mL) and dried under high vacuum overnight to afford the title compound (40 mg) as white solid. LC-MS retention time=2.69 min; m/z=380.20 [M+H]⁺. (Column: Phenomenex C18 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Intermediate 170

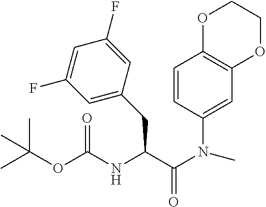 [SMILES: CN(C(=O)[C@H](Cc1cc(F)cc(F)c1)NC(=O)OC(C)(C)C)c1ccc2c(c1)OCCO2]

HATU (127 mg, 0.33 mmol) was added to a mixture of (S)-2-((tert-butoxycarbonyl)amino)-3-(3,5-difluorophenyl)propanoic acid (100 mg, 0.33 mmol) and N-methyl-2,3-dihydrobenzo[b][1,4]dioxin-6-amine (50 mg, 0.30 mmol) in DMF (2 mL) and DIPEA (0.16 mL, 0.91 mmol) and the reaction mixture was stirred at rt for 2 h. The reaction was filtered, and purified by preparative HPLC to afford the title compound (125 mg). LC-MS retention time=1.94 min; m/z=449.2 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Intermediate 171

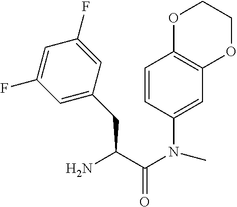 [SMILES: CN(C(=O)[C@@H](N)Cc1cc(F)cc(F)c1)c1ccc2c(c1)OCCO2]

A solution of 4 M HCl in dioxane (1.5 mL, 6.0 mmol) was added to a solution of Intermediate 170 (125 mg, 0.28 mmol) in MeOH (1.5 mL) and the reaction mixture was stirred at rt for 16 h. The reaction was concentrated and the residue was azeotroped with EtOH and dried to afford an HCl salt of the title compound (119 mg) as yellow solid. LC-MS retention time=0.87 min; m/z=349.2 [M+H]⁺. (Column: Waters Aquity BEH C18 2.1×50 mm 1.7 U. Solvent A=100% Water: 0.05% TFA. Solvent B=100% Acetonitrile: 0.05% TFA. Flow Rate=0.8 mL/min. Gradient: 2-98% B. Gradient Time=1.5 min. Wavelength=220).

### Intermediate 172

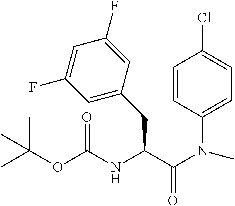 [SMILES: CN(C(=O)[C@H](Cc1cc(F)cc(F)c1)NC(=O)OC(C)(C)C)c1ccc(Cl)cc1]

HATU (148 mg, 0.39 mmol) was added to a mixture of (S)-2-((tert-butoxycarbonyl)amino)-3-(3,5-difluorophenyl)propanoic acid (117 mg, 0.39 mmol) and 4-chloro-N-methylaniline (50 mg, 0.35 mmol) in DMF (2 mL) and DIPEA (0.18 mL, 1.1 mmol) and the reaction mixture was stirred at rt for 2 h. The reaction was filtered, and purified by preparative HPLC to afford the title compound (108.7 mg). LC-MS retention time=2.37 min; m/z=425.0 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Intermediate 173

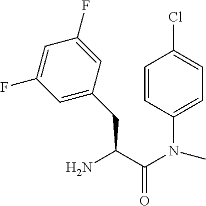 [SMILES: CN(C(=O)[C@@H](N)Cc1cc(F)cc(F)c1)c1ccc(Cl)cc1]

A solution of 4M HCl in dioxane (1.5 mL, 6.0 mmol) was added to a solution of Intermediate 172 (108 mg, 0.25 mmol) in MeOH (1.5 mL) and the reaction mixture was stirred at rt for 16 h. The reaction was concentrated and the residue was azeotroped with EtOH and dried to afford an HCl salt of title the compound (108 mg) as yellow solid. LC-MS retention time=0.91 min; m/z=325.1 [M+H]⁺. (Column: Waters Aquity BEH C18 2.1×50 mm 1.7 U. Solvent A=100% Water: 0.05% TFA. Solvent B=100% Acetonitrile: 0.05% TFA. Flow Rate=0.8 mL/min. Gradient: 2-98% B. Gradient Time=1.5 min. Wavelength=220).

### Intermediate 174

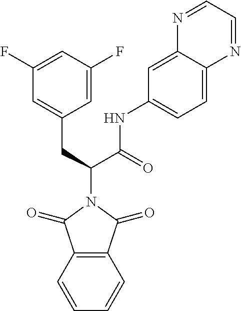 [SMILES: O=C(Nc1ccc2nccnc2c1)[C@H](Cc1cc(F)cc(F)c1)N1C(=O)c2ccccc2C1=O]

HATU (0.631 g, 1.66 mmol) was added to a mixture of Intermediate 125 (0.50 g, 1.5 mmol) and quinoxalin-6-amine (0.219 g, 1.51 mmol) in DMF (10 mL) and DIPEA (0.53 mL, 3 mmol) and the reaction mixture was stirred at rt for 16 h. The reaction mixture was partitioned between aq NaHCO₃-NaCl (30 mL) and EtOAc (2×50 mL) and the combined organic components were dried, filtered concentrated and then purified by flash silica chromatography: (40 g SiO₂, eluted with solv A=Hexane/solv B=EtOAc, gradient from 0-70% B, hold at 70% B) to yield the title compound (790 mg) as light yellow solid. LC-MS retention time=1.15 min; m/z=459.1 [M+H]⁺. (Column: Waters Aquity BEH C18 2.1×50 mm 1.7 U. Solvent A=100% Water: 0.05% TFA. Solvent B=100% Acetonitrile: 0.05% TFA. Flow Rate=0.8 mL/min. Gradient: 2-98% B. Gradient Time=1.5 min. Wavelength=220).

### Intermediate 175

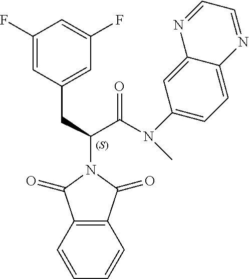

60% NaH (0.019 g, 0.48 mmol) as a dispersion in mineral oil was added to a solution of Intermediate 174 (0.2 g, 0.4 mmol) in THF (5 mL). The reaction solution was then treated with MeI (0.030 mL, 0.48 mmol) and the mixture was stirred at rt for 16 h. The reaction was slowly quenched with aq. NH₄Cl (10 mL) and then extracted with EtOAc (2×20 mL). The organic components were combined, dried and concentrated to afford the title compound as orange solid. LC-MS retention time=1.11 min; m/z=473.0 [M+H]⁺. (Column: Waters Aquity BEH C18 2.1×50 mm 1.7 U. Solvent A=100% Water: 0.05% TFA. Solvent B=100% Acetonitrile: 0.05% TFA. Flow Rate=0.8 mL/min. Gradient: 2-98% B. Gradient Time=1.5 min. Wavelength=220).

### Intermediate 176

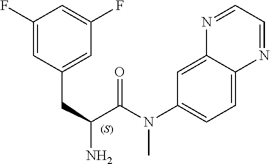

Hydrazine (0.062 mL, 1.7 mmol) was added to a mixture of Intermediate 175 (0.20 g, 0.2 mmol) in EtOH (5 mL) and the reaction mixture was stirred at 50° C. for 16 h. The reaction was filtered to remove solids and the filtrate was concentrated and purified by preparative HPLC to afford the title compound (44.8 mg). LC-MS retention time=1.28 min; m/z=343.0 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Intermediate 177

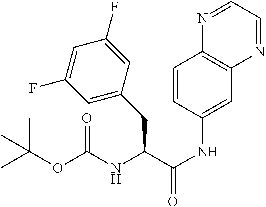 [SMILES: CC(C)(C)OC(=O)N[C@@H](Cc1cc(F)cc(F)c1)C(=O)Nc1ccc2nccnc2c1]

To a mixture of (S)-2-((tert-butoxycarbonyl)amino)-3-(3,5-difluorophenyl)propanoic acid (500 mg, 1.66 mmol) and quinoxalin-6-amine (219 mg, 1.5 mmol) in DMF (7 mL), DIPEA (0.80 mL, 4.5 mmol) was added, followed by HATU (631 mg, 1.66 mmol) and then the reaction mixture was stirred at rt for 16 h. The reaction mixture was partitioned between aq. NaHCO₃-NaCl (10 mL) and EtOAc (3×25 mL), and the combined organic components were dried (Na₂SO₄), filtered, concentrated and then purified by flash silica chromatography (24 g Silica, eluted with solv A=Hexane/solv B=EtOAc, gradient from 0-50% B, hold at 50% B) to afford the title compound (515 mg). LC-MS retention time=1.15 min; m/z=429.0 [M+H]⁺. (Column: Waters Aquity BEH C18 2.1×50 mm 1.7 U. Solvent A=100% Water: 0.05% TFA. Solvent B=100% Acetonitrile: 0.05% TFA. Flow Rate=0.8 mL/min. Gradient: 2-98% B. Gradient Time=1.5 min. Wavelength=220). ¹H NMR (400 MHZ, chloroform-d) δ 8.96-8.65 (m, 3H), 8.31 (d, J=2.3 Hz, 1H), 8.01 (d, J=9.0 Hz, 1H), 7.81 (dd, J=9.0, 2.3 Hz, 1H), 6.83 (d, J=6.0 Hz, 2H), 6.77-6.66 (m, 1H), 5.19 (d, J=6.8 Hz, 1H), 4.58 (d, J=6.3 Hz, 1H), 3.30 (dd, J=14.1, 6.5 Hz, 1H), 3.20-3.05 (m, 1H), 1.46 (s, 9H).

### Intermediate 178

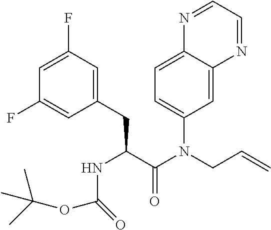 [SMILES: C=CCN(C(=O)[C@H](Cc1cc(F)cc(F)c1)NC(=O)OC(C)(C)C)c1ccc2nccnc2c1]

To a mixture of Intermediate 177 (200 mg, 0.46 mmol) in DMF (3 mL), 3-bromoprop-1-ene (0.06 mL, 0.7 mmol) was added, followed by 60% NaH (20 mg, 0.51 mmol) as a dispersion in mineral oil and the reaction mixture was stirred at rt for 3 h. The reaction mixture was partitioned between aq. NH₄Cl-NaCl (10 mL) and EtOAc (3×15 mL), and the combined organic components were dried (Na₂SO₄), filtered, concentrated and then purified by flash silica chromatography (12 g Silica, eluted with solv A=Hexane/solv B=EtOAc, gradient from 0-50% B, hold at 50% B) to afford the title compound (176 mg). ¹H NMR (400 MHZ, chloroform-d) δ 8.91 (br. s., 2H), 8.14 (d, J=8.8 Hz, 1H), 7.60 (br. s., 1H), 7.32 (br. s., 1H), 6.71 (t, J=8.4 Hz, 1H), 6.47 (d, J=5.0 Hz, 2H), 5.85 (ddd, J=17.0, 6.5, 3.9 Hz, 1H), 5.22 (d, J=7.5 Hz, 1H), 5.17 (d, J=10.0 Hz, 1H), 5.08 (d, J=17.3 Hz, 1H), 4.49 (d, J=6.5 Hz, 1H), 4.36 (d, J=5.8 Hz, 2H), 2.94 (dd, J=12.9, 8.2 Hz, 1H), 2.76 (dd, J=12.9, 5.4 Hz, 1H), 1.40 (br. s., 9H).

### Intermediate 179

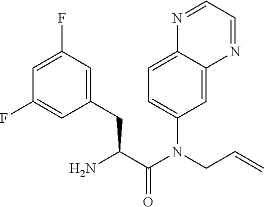 [SMILES: C=CCN(C(=O)[C@@H](N)Cc1cc(F)cc(F)c1)c1ccc2nccnc2c1]

To a mixture of Intermediate 178 (172 mg, 0.36 mmol) in MeOH (1.5 mL), 4M HCl in dioxane (1.5 mL, 6 mmol) was added and the reaction mixture was stirred at rt for 4 h. The reaction mixture was concentrated and the residue was azeotroped with toluene to afford an HCl salt of the title compound (160 mg) which was used without additional purification. LC-MS retention time=0.86 min; m/z=369.2 [M+H]⁺. (Column: Waters Aquity BEH C18 2.1×50 mm 1.7 U. Solvent A=100% Water: 0.05% TFA. Solvent B=100% Acetonitrile: 0.05% TFA. Flow Rate=0.8 mL/min. Gradient: 2-98% B. Gradient Time=1.5 min. Wavelength=220).

### Intermediate 180

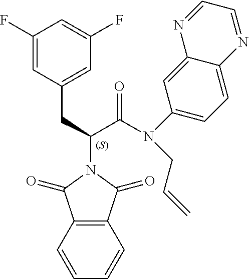

3-Bromoprop-1-ene (0.28 mL, 3.3 mmol) was added to a solution of Intermediate 174 (0.30 g, 0.65 mmol) and Cs₂CO₃ (0.43 g, 1.3 mmol) in DMF (5 mL) and the reaction mixture was stirred at 70° C. for 2 h, then at rt overnight. 60% NaH (0.039 g, 0.98 mmol) as a dispersion in mineral oil and additional 3-bromoprop-1-ene (0.28 mL, 3.3 mmol) were added and the stirring was continued at rt for 16 h. The reaction mixture was filtered and purified by preparative HPLC to afford the title compound (0.18 g). LC-MS retention time=1.19 min; m/z=499.1 [M+H]⁺. (Column: Waters Aquity BEH C18 2.1×50 mm 1.7 U. Solvent A=100% Water: 0.05% TFA. Solvent B=100% Acetonitrile: 0.05% TFA. Flow Rate=0.8 mL/min. Gradient: 2-98% B. Gradient Time=1.5 min. Wavelength=220).

### Intermediate 181

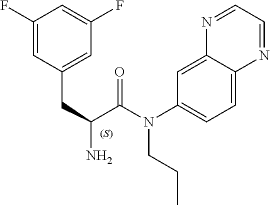

Hydrazine (0.018 mL, 0.48 mmol) was added to a solution of Intermediate 180 (90 mg, 0.12 mmol) in EtOH (5 mL) and the reaction mixture was stirred at 50° C. for 16 h. The reaction mixture was filtered and the filtrate was concentrated and purified by preparative HPLC to afford the title compound (18.8 mg). LC-MS retention time=1.53 min; m/z=371.0 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Intermediate BB-1

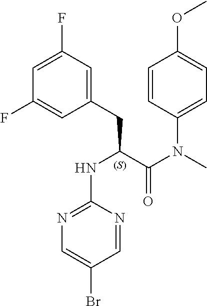

To a solution of Intermediate 13 (260 mg, 0.812 mmol) in dioxane (10 mL) was added 5-bromo-2-chloropyrimidine (157 mg, 0.812 mmol), BINAP (76 mg, 0.12 mmol), cesium carbonate (529 mg, 1.62 mmol) and reaction mixture was degasified with nitrogen gas for 5 min. Pd(OAc)₂ (14.6 mg, 0.065 mmol) was added the reaction mixture was heated to reflux and stirred for 5 h. The reaction mixture was cooled RT, diluted with saturated aqueous NH₄Cl solution (50 mL) and extracted with EtOAc (2×50 mL). The combined organic layer was washed with brine (75 mL), dried (Na₂SO₄), filtered, concentrated and the crude product was purified by combiflash chromatography (24 g Redisep® SiO₂ column, eluting with 40% EtOAc in n-hexanes) to afford the title compound (0.129 g) as a red liquid. LC-MS retention time=3.11 min; m/z=477.0 [M+H]⁺. Column: KINETIX XB-C18, 75×3 mm, 2.6 μm; Flow rate: 1 mL/min; Mobile Phase A: 10 mM HCOONH₄ in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm.

### Intermediate BB-2

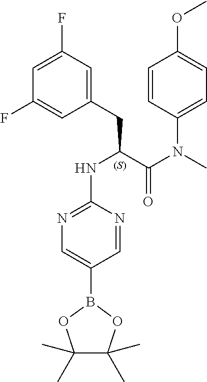

To a solution of Intermediate BB-1 (150 mg, 0.314 mmol) in dioxane (15 mL) was added potassium acetate (61.7 mg, 0.629 mmol), bis(pinacolato)diboran (160 mg, 0.629 mmol) and reaction mixture was degasified with nitrogen for 10 min. PdCl₂(dppf).CH₂Cl₂ adduct (20.5 mg, 0.025 mmol) was added to the above reaction mixture and heated to 100° C. and stirred for 5 h. The reaction mixture was cooled RT, diluted with saturated aqueous NH₄Cl solution (50 mL) and extracted with EtOAc (2×50 mL). The combined organic layer was washed with brine (75 mL), dried (Na₂SO₄), filtered and concentrated to afford the title compound (0.3 g) as pale yellow solid which was taken to next reaction without further purification. LC-MS retention time=3.45 min; m/z=525.2 [M+H]⁺. Column: KINETIX XB-C18, 75×3 mm, 2.6 μn; Flow rate: 1 mL/min; Mobile Phase A: 10 mM HCOONH₄ in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm.

### Intermediate BB-3

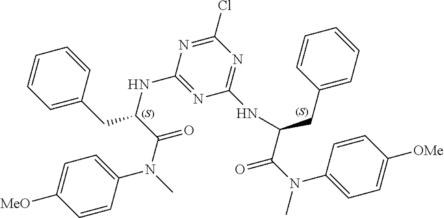

To a stirred solution of Intermediate 4 (100 mg, 0.35 mmol) in THF (10 mL) was added 2,4,6-trichloro-1,3,5-triazine (64.9 mg, 0.35 mmol), followed by DIPEA (0.18 mL, 1.0 mmol) and the reaction mixture was stirred at 80° C. for 16 h. The reaction mixture was diluted with water (20 mL) and extracted with DCM (3×20 mL). The combined organic layer was washed with water (20 mL), brine (20 mL), dried (Na₂SO₄), filtered and concentrated. The crude product was purified by combiflash chromatography (40 g Redisep® SiO₂ column, eluting with 0-20% EtOAc in hexanes) to afford the title compound (60 mg) as an off white solid. LC-MS retention time=1.2 min; m/z=680.5 [M+H]⁺. Column: Acquity BEH C8 (2.1×50 mm) 1.7μ: Flow rate: 0.8 mL/min; Mobile Phase A: 10 mM HCO₂NH₄ in water: ACN (95:5); Mobile Phase B: 10 mM HCO₂NH₄ in water: ACN (5:95); 5% B to 95% B over 1.1 minutes and then hold a 0.6 min. at 95% B of flow rate 0.8 mL/min; Detection: UV at 220 nm.

### Intermediate BB-4

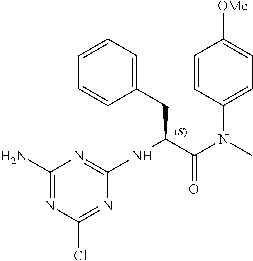

To a stirred solution of Intermediate 4 (100 mg, 0.35 mmol) in THF (10 mL) was added 4,6-dichloro-1,3,5-triazin-2-amine (58 mg, 0.35 mmol) followed by DIPEA (0.18 mL, 1.0 mmol) and the reaction mixture was stirred at 80° C. for 16 h. The reaction mixture was diluted with water (20 mL) and extracted with DCM (3×20 mL). The combined organic layer was washed with water (20 mL), brine (20 mL), dried (Na₂SO₄), filtered and concentrated. The crude product was purified by preparative HPLC to afford the title compound (16 mg) as an off white solid. LC-MS retention time=1.60 min; m/z=413.2 [M+H]⁺. Column: Ascentis Express C18 (50×2.1) mm, 2.7 μm; Flow: 1.1 mL/min; Mobile Phase A: 10 mM NH₄OAc in water: ACN (95:5); Mobile Phase B: 10 mM NH₄OAc in water: ACN (5:95); Temperature: 50° C.; 0% B to 100% B over 3 minutes; UV Detection at 220 nm.

### Intermediate BB-5

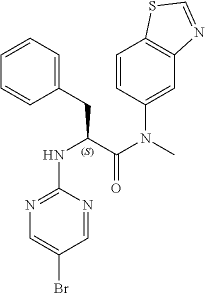

To a stirred solution of Intermediate 18 (1.00 g, 3.21 mmol) in dioxane (20 mL) was added cesium carbonate (2.62 g, 8.03 mmol), 5-bromo-2-iodopyrimidine (1.10 g, 3.85 mmol), BINAP (0.300 g, 0.482 mmol) and reaction mixture was degasified with nitrogen for 10 min. Pd(OAc)₂ (0.058 g, 0.257 mmol) was added to the above reaction mixture and heated to reflux for 8 h. The reaction mixture was cooled RT, diluted with saturated NH₄Cl solution (50 mL) and extracted with EtOAc (2×50 mL). The combined organic layer was washed with brine (75 mL), dried (Na₂SO₄), filtered and concentrated. The crude product was purified by combiflash chromatography (24 g Redisep® SiO₂ column, eluting with 35% EtOAc in n-hexanes) to afford the title compound (1.1 g) as a pale red solid. LC-MS retention time=2.72 min; m/z=468.0 [M+H]⁺. Column: KINETIX XB-C18, 75×3 mm, 2.6 μm; Flow rate: 1 mL/min; Mobile Phase A: 10 mM HCOONH₄ in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm. ¹H NMR (400 MHZ, DMSO-d₆) δ 9.49 (s, 1H), 8.34-8.23 (m, 3H), 8.11 (s, 1H), 7.75 (d, J=7.5 Hz, 1H), 7.53 (d, J=8.0 Hz, 1H), 7.06 (m, 3H), 6.80 (d, J=4.0 Hz, 2H), 4.50 (br. s., 1H), 3.24 (s, 3H), 3.02 (dd, J=13.6, 4.0 Hz, 1H), 2.89-2.79 (m, 1H).

### Intermediate BB-6

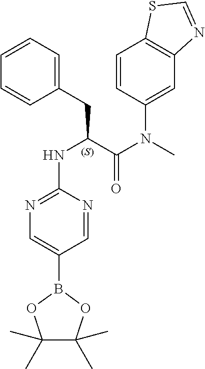

To a stirred solution of Intermediate BB-5 (1.00 g, 2.14 mmol) in dioxane (20 mL) was added bis(pinacolato)diboron (0.651 g, 2.56 mmol), potassium acetate (0.419 g, 4.27 mmol) and the reaction mixture was degasified with nitrogen for 10 min. PdCl₂(dppf) CH₂Cl₂ adduct (0.139 g, 0.171 mmol) was added to the above reaction mixture and stirred at 100° C. for 16 h. The reaction mixture was cooled to RT; diluted with saturated NH₄Cl solution (50 mL) and extracted with EtOAc (2×50 mL). The combined organic layer was washed with brine (75 mL), dried (Na₂SO₄), filtered and concentrated to afford the title compound (0.3 g) as pale yellow solid which was taken to next reaction without further purification. LC-MS retention time=3.09 min; m/z=516.2 [M+H]⁺. Column: KINETIX XB-C18, 75×3 mm, 2.6 μm; Flow rate: 1 mL/min; Mobile Phase A: 10 mM HCOONH₄ in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm. ¹H NMR (300 MHz, DMSO-d₆) δ 9.49 (s, 1H), 8.36 (br. s., 2H), 8.27 (d, J=8.3 Hz, 1H), 8.17 (br. s., 1H), 7.88 (d, J=8.3 Hz, 1H), 7.56 (br. s., 1H), 7.07 (br. s., 3H), 6.84 (br. s., 2H), 4.55 (br. s., 1H), 3.23 (s, 3H), 3.07-2.98 (m, 1H), 2.94-2.82 (m, 1H), 1.27 (s, 12H).

### Intermediate BB-7

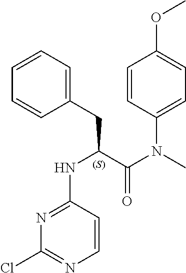

To a solution of Intermediate 4 (1.20 g, 4.22 mmol) in 2-propanol (20 mL) was added 2,4-dichloropyrimidine (0.629 g, 4.22 mmol), DIPEA (3.69 mL, 21.10 mmol) and the reaction mixture was heated to reflux and stirred for 2 h. The reaction mixture was diluted with saturated NH₄Cl solution (50 mL) and extracted with EtOAc (2×50 mL). The combined organic layer was washed with brine (75 mL), dried (Na₂SO₄), filtered and concentrated. The crude product was purified by combiflash chromatography (24 g Redisep® SiO₂ column, eluting with 30% EtOAc in n-hexanes) to afford the title compound (0.8 g) as an off white solid. LC-MS retention time=2.49 min; m/z=397.0 [M+H]⁺. Column: KINETIX XB-C18, 75×3 mm, 2.6 win; Flow rate: 1 mL/min; Mobile Phase A: 10 mM HCOONH₄ in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm. ¹H NMR (300 MHZ, DMSO-d₆) δ 8.20 (d, J=4.9 Hz, 1H), 7.95 (d, J=5.7 Hz, 1H), 7.37 (d, J=5.7 Hz, 2H), 7.18-7.10 (m, 3H), 7.05 (d, J=9.1 Hz, 2H), 6.85 (d, J=6.0 Hz, 2H), 6.69 (d, J=5.3 Hz, 1H), 4.60-4.40 (m, 1H), 3.82 (s, 3H), 3.14 (s, 3H), 2.96 (dd, J=13.8, 4.2 Hz, 1H), 2.72 (dd, J=13.2,10.2 Hz, 1H).

### Intermediate BB-8

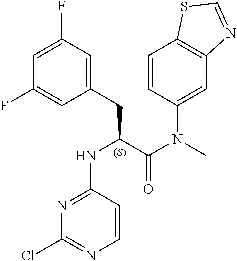

To a stirred solution of Intermediate 20 (120 mg, 0.345 mmol) in 2-propanol (5 mL) was added DIPEA (0.181 mL, 1.036 mmol), 2,4-dichloropyrimidine (61.8 mg, 0.415 mmol) and the reaction mixture was stirred at 100° C. for 10 h. The reaction mixture was cooled RT, diluted with saturated NH₄Cl solution (50 mL) and extracted with EtOAc (2×50 mL). The combined organic layer was washed with brine (75 mL), dried (Na₂SO₄), filtered, concentrated and the crude material was purified by preparative LC/MS to afford the title product (32 mg) as a pale yellow solid. LC-MS retention time=1.59 min; m/z=460.2 [M+H]⁺. Column: Ascentis Express C18 (50×2.1) mm, 2.7 μm; Flow: 1.1 mL/min; Mobile Phase A: 10 mM NH₄OAc in water: ACN (95:5); Mobile Phase B: 10 mM NH₄OAc in water: ACN (5:95); Temperature: 50° C.; 0% B to 100% B over 3 minutes; UV Detection at 220 nm. ¹H NMR (400 MHZ, DMSO-d₆) δ 9.51 (s, 1H), 8.40 (d, J=8.0 Hz, 1H), 8.31 (d, J=8.5 Hz, 1H), 8.23 (br. s., 1H), 7.90 (d, J=5.5 Hz, 1H), 7.64 (d, J=8.0 Hz, 1H), 6.97 (t, J=9.5 Hz, 1H), 6.49 (dd, J=14.1, 6.5 Hz, 3H), 4.61 (br. s., 1H), 3.29 (s, 3H), 3.10 (dd, J=13.8, 3.8 Hz, 1H), 2.83 (dd, J=13.6, 10.0 Hz, 1H).

### Intermediate BB-9

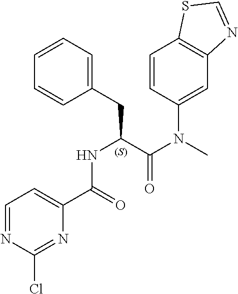

To a solution of Intermediate 18 (1.60 g, 5.14 mmol) and 2-chloropyrimidine-4-carboxylic acid (0.978 g, 6.17 mmol) in DMF (8 mL) at room temperature was added DIPEA (2.69 mL, 15.4 mmol), HATU (2.34 g, 6.17 mmol) and the reaction mixture was stirred for 16 h. The reaction mixture was concentrated to dryness and the crude product was purified by combiflash chromatography (24 g Redisep® SiO₂ column, eluting with 30% EtOAc in n-hexanes) to afford the title compound (600 mg) as a yellow solid. LC-MS retention time=2.49 min; m/z=452.0 [M+H]⁺. Column: KINETIX XB-C18, 75×3 mm, 2.6 μm; Flow rate: 1 mL/min; Mobile Phase A: 10 mM HCOONH₄in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm. ¹H NMR (300 MHZ, D₂O, DMSO-d₆) δ 9.46 (s, 1H), 8.97 (d, J=4.8 Hz, 1H), 8.23 (d, J=9 HZ, 1H), 7.87 (m, 2H), 7.30 (d, J=8.4 Hz, 1H), 7.20-7.10 (m, 3H), 6.84 (d, J=6.6 Hz, 1H), 4.69 (m, 1H), 3.25 (s, 3H), 3.01-2.89 (m, 2H).

### Intermediate BB-10

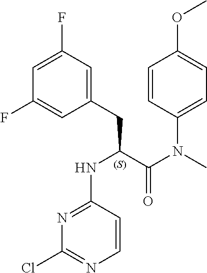

To a stirred solution of Intermediate 13 (1.00 g, 3.12 mmol) in 2-propanol (20 mL) was added 2,4-dichloropyrimidine (465 mg, 3.12 mmol), DIPEA (2.73 mL, 15.61 mmol) and the reaction mixture was heated to reflux for 5 h. The reaction mixture was diluted with saturated NH₄Cl solution (50 mL) and extracted with EtOAc (2×50 mL). The combined organic layer was washed with brine (75 mL), dried (Na₂SO₄), filtered, concentrated and the crude product was purified by combiflash chromatography (24 g Redisep® SiO₂ column, eluting with 30% EtOAc in n-hexanes) to afford the title compound (0.8 g) as an off white solid. LC-MS retention time=2.98 min; m/z=433.0 [M+H]⁺. Column: KINETIX XB-C18, 75×3 mm, 2.6 μm; Flow rate: 1 mL/min; Mobile Phase A: 10 mM HCOONH₄ in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm. ¹H NMR (300 MHZ, DMSO-d₆) δ 8.22 (d, J=4.9 Hz, 1H), 8.06-7.93 (m, 1H), 7.58-7.42 (m, 2H), 7.08 (d, J=8.7 Hz, 2H), 7.03-6.94 (m, 1H), 6.71 (d, J=5.3 Hz, 1H), 6.48 (br. s., 2H), 4.56-4.37 (m, 1H), 3.33 (s, 3H), 3.16 (s, 3H), 2.93-2.83 (m., 2H).

### Intermediate BB-13

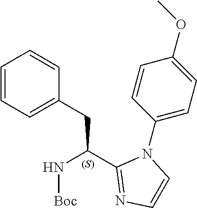

To a stirred solution of (S)-tert-butyl (1-oxo-3-phenylpropan-2-yl)carbamate (1.00 g, 4.01 mmol) and 4-methoxyaniline (0.49 g, 4.01 mmol) in MeOH (20mL) was added ammonium acetate (0.31 g, 4.01 mmol) followed by hexahydro-[1,4]dioxino[2,3-b][1,4]dioxine-2,3,6,7-tetraol (0.843 g, 4.01 mmol) and the reaction mixture was stirred at room temperature for 2 h. The reaction mixture was diluted with EtOAc (100 mL) and washed with water (25 mL), brine (25 mL), dried (Na₂SO₄), filtered and concentrated. The crude product was purified by preparative HPLC to afford the title compound (30 mg) as an off white solid. LC-MS retention time=2.87 min; m/z=394.2 [M+H]⁺. Column: KINETIX XB-C18, 75×3 mm, 2.6 μm; Flow rate: 1 mL/min; Mobile Phase A: 10 mM HCOONH₄ in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm.

### Intermediate BB-14

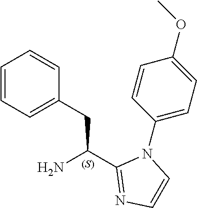

To a Intermediate BB-13 (30 mg, 0.08 mmol) was added a solution of 4 M HCl (381 μL, 1.53 mmol) in dioxane and stirred at room temperature for 16 h. The reaction mixture was concentrated to dryness to afford the HCl salt of the title compound (25 mg) as brown solid. LC-MS retention time=1.95 min; m/z=294.2 [M+H]⁺. Column: KINETIX XB-C18, 75×3 mm, 2.6 μm; Flow rate: 1 mL/min; Mobile Phase A: 10 mM HCOONH₄ in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm. ¹H NMR (300 MHz, DMSO-d₆) δ 8.78 (br. s., 2H), 7.30 (d, J=7.6 Hz, 2H), 7.26-7.16 (m, 3H), 6.90 (d, J=9.1 Hz, 2H), 6.76 (d, J=6.4 Hz, 2H), 6.64 (d, J=9.1 Hz, 2H), 4.22 (br. s., 1H), 3.78 (s, 3H), 3.23-3.18 (m, 2H)

### Intermediate BB-15

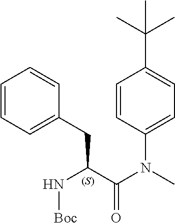

To a stirred solution of (S)-2-((tert-butoxycarbonyl)amino)-3-phenylpropanoic acid (200 mg, 0.754 mmol) in DMF (5 mL) was added HATU (430 mg, 1.131 mmol), DIPEA (0.395 mL, 2.262 mmol) and the reaction mixture was stirred for 30 min. 4-(tert-Butyl)-N-methyl aniline (148 mg, 0.905 mmol) was added to the above reaction mixture and stirred at room temperature for 16 h. The reaction mixture was then diluted with water (20 mL) and extracted with EtOAc (3×25 mL). The combined organic layer was washed with 10% aqueous NaHCO₃ solution (25 mL), water (25 mL), brine (25 mL), dried (Na₂SO₄), filtered and concentrated. The crude product was purified by combiflash chromatography (24 g Redisep® SiO₂ column, eluting with 0-15% EtOAc in hexanes) to afford the title compound (0.12 g) as an off white solid. LC-MS retention time=1.44 min; m/z=411.3 [M+H]⁺. Column: Acquity BEH C8 (2.1×50 mm) 1.7 μm: Flow rate: 0.8 mL/min; Mobile Phase A: 5 mM NH₄OAc in water: ACN (95:5); Mobile Phase B: 5 mM NH₄OAc in water: ACN (5:95); 5% B to 95% B over 1.1 minutes and then hold a 0.6 min. at 95% B of flow rate 0.8 mL/min; Detection: UV at 220 nm. ¹H NMR (300 MHz, CDCl₃) δ 7.38 (d, J=7.93 Hz, 2H), 7.23-7.13 (m, 3H), 6.87 (d, J=8.2 Hz, 2H), 6.81 (m, 2H), 5.30-5.17 (m, 1H), 4.69-4.56 (m, 1H), 3.24 (s, 3H), 2.84 (dd, J=13.8, 6.6 Hz, 1H), 2.62 (dd, J=12.6, 7.5 Hz, 1H), 1.37 (s, 9H), 1.34 (s, 9H).

### Intermediate BB-16

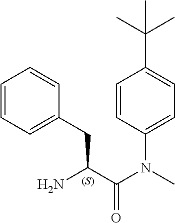

Intermediate BB-15 (150 mg, 0.365 mmol) was added HCl (457 μL, 1.827 mmol, 4M in dioxane) and reaction mixture was stirred at room temperature for 4 h. The reaction mixture was concentrated to dryness; the residue was triturated with hexane (3×50 mL) followed by azeotropic distillation of solid product with toluene (2×25 mL) to afford the title compound (0.11 g) as an off white solid. LC-MS retention time=1.12 min; m/z=311.2 [M+H]⁺. Column: Acquity BEH C8 (2.1×50 mm) 1.7μ: Flow rate: 0.8 mL/min; Mobile Phase A: 5 mM NH₄OAc in water: ACN (95:5); Mobile Phase B: 5 mM NH₄OAc in water: ACN (5:95); 5% B to 95% B over 1.1 minutes and then hold a 0.6 min. at 95% B of flow rate 0.8 mL/min; Detection: UV at 220 nm. ¹H NMR (300 MHZ, DMSO-d₆) δ 8.31 (br. s., 2H), 7.42 (d, J=8.7 Hz, 2H), 7.30-7.20 (m, 3H), 6.97 (m, 2H), 6.74 (d, J=6.0 Hz, 2H), 3.84 (br. s., 1H), 3.17 (s, 3H), 2.91 (dd, J=12.6, 6.8 Hz, 1H), 2.75 (dd, J=13.8, 8.8 Hz, 1H), 1.31 (s, 9H).

### Intermediate BB-17

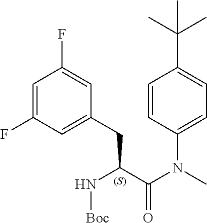

To a stirred solution of (S)-2-((tert-butoxycarbonyl)amino)-3-(3,5-difluorophenyl)propanoic acid (150 mg, 0.498 mmol) in DMF (5 mL) was added HATU (284 mg, 0.747 mmol), DIPEA (0.261 mL, 1.49 mmol) and the reaction mixture was stirred for 30 minutes. 4-(tert-Butyl)-N-methyl aniline (98 mg, 0.60 mmol) was added to the above reaction mixture and stirred at room temperature for 16 h. The reaction mixture was diluted with water (20 mL) and extracted with EtOAc (3×25 mL). The combined organic layer was washed with 10% aqueous NaHCO₃ solution (25 mL), water (25 mL), brine (25 mL), dried (Na₂SO₄), filtered and concentrated. The crude product was purified by combiflash chromatography (24 g Redisep® SiO₂ column, eluting with 0-15% EtOAc in hexanes) to afford the title compound (0.12 g) as a brown color liquid. LC-MS retention time=1.36 min; m/z=447.3 [M+H]⁺. Column: Acquity BEH C8 (2.1×50 mm) 1.7 μm: Flow rate: 0.8 mL/min; Mobile Phase A: 5 mM NH₄OAc in water: ACN (95:5); Mobile Phase B: 5 mM NH₄OAc in water: ACN (5:95); 5% B to 95% B over 1.1 minutes and then hold a 0.6 min. at 95% B of flow rate 0.8 mL/min; Detection: UV at 220 nm. ¹H NMR (300 MHZ, CDCl₃) δ 7.45 (d, J=8.3 Hz, 2H), 7.00 (d, J=8.3 Hz, 2H), 6.63 (t, J=9.1 Hz, 1H), 6.31 (d, J=6.8 Hz, 2H), 5.30 (d, J=14.7 Hz, 1H), 4.63 (m, 1H), 3.27 (s, 3H), 2.82 (dd, J=13.4, 5.5 Hz, 1H), 2.64-2.53 (dd, J=13.4, 7.8 Hz, 1H), 1.38 (s, 9H), 1.34 (s, 9H).

### Intermediate BB-18

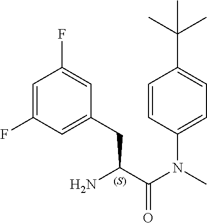

A solution of Intermediate BB-17 (120 mg, 0.269 mmol) was added HCl in dioxane (4M solution, 336 μl, 1.34 mmol) reaction mixture was stirred at room temperature for 4 h. The reaction mixture was concentrated to dryness; the residue was triturated with hexane (3×50 mL) followed by azeotropic distillation with toluene (2×25 mL) afford the title compound (0.10 g) as an off white solid. LC-MS retention time=3.25 min; m/z=347.2 [M+H]⁺. Column: KINETIX XB-C18, 75×3 mm, 2.6 μm; Flow rate: 1 mL/min; Mobile Phase A: 10 mM HCOONH₄ in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm. ¹H NMR (300 MHz, DMSO-d₆) δ 8.37 (br. s., 2H), 7.49 (d, J=8.69 Hz, 2H), 7.17 (d, J=8.1 Hz, 2H), 7.05 (m, 1H), 6.41 (d, J=6.04 Hz, 2H), 3.90 (m, 1H), 3.20 (s, 3H), 3.01-2.76 (m, 2H), 1.31 (s, 9H).

### Intermediate BB-19

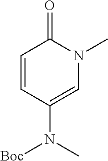 [SMILES: CN(C(=O)OC(C)(C)C)c1ccc(=O)n(C)c1]

To a stirred solution of tert-butyl (1-methyl-6-oxo-1,6-dihydropyridin-3-yl)carbamate (1 g, 4.46 mmol) in DMF (10 mL) at 0° C. was added portion wise 60% NaH (0.446 g, 11.15 mmol, dispersion in mineral oil) and the reaction mixture was stirred at room temperature for 20 min. Methyl iodide (0.418 mL, 6.69 mmol) was added drop wise at the same temperature and the reaction mixture was stirred at room temperature for 16 h. The reaction mixture was diluted with water (50 mL) and extracted with EtOAc (3×30 mL). The combined organic layer was washed with water (50 mL), brine (50 mL), dried (Na₂SO₄), filtered and concentrated to afford the title compound (1 g) as pale yellow oil. LC-MS retention time=1.34 min; m/z=239.2 [M+H]⁺. Column: KINETIX XB-C18, 75×3 mm, 2.6 μm; Flow rate: 1 mL/min; Mobile Phase A: 10 mM HCOONH₄ in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm. ¹H NMR (300 MHZ, DMSO-d₆) δ 7.75 (d, J=2.6 Hz, 1H), 7.38 (dd, J=9.8, 3.0 Hz, 1H), 6.34 (d, J=9.4 Hz, 1H), 3.33 (s, 3H), 3.07 (s, 3H), 1.37 (s, 9H).

### Intermediate BB-20

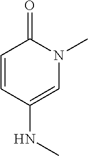

To a stirred solution of Intermediate BB-19 (1.0 g, 4.20 mmol) in dioxane (2 mL) was added 4 M HCl in dioxane (6 mL, 4.20 mmol) and stirred at room temperature for 3 h. The reaction mixture was concentrated to dryness; the residue was triturated with diethyl ether (3×50 mL) and the resulting solid was allowed to settle and the supernatant was decanted to afford the title compound as an off white solid (0.5 g) as an off white solid. LC-MS retention time=0.45 min; m/z=139.2 [M+H]⁺. Column: KINETIX XB-C18, 75×3 mm, 2.6 μm; Flow rate: 1 mL/min; Mobile Phase A: 10 mM HCOONH₄ in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm. ¹H NMR (400 MHZ, DMSO-d₆) δ 11.67-10.44 (br. s., 1H), 8.03 (d, J=2.5 Hz, 1H), 7.64 (dd, J=10.0, 3.0 Hz, 1H), 6.56 (d, J=9.5 Hz, 1H), 3.46 (s, 3H), 2.80 (s, 3H).

### Intermediate BB-21

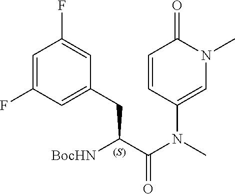

To a stirred solution of (S)-2-((tert-butoxycarbonyl)amino)-3-(3,5-difluorophenyl)propanoic acid (0.250 g, 0.830 mmol) in DMF (2 mL) at 0° C. was added HATU (0.38 g, 1 mmol) and DIPEA (0.73 mL, 4.15 mmol) and the reaction mixture was stirred for 30 min. Intermediate BB-20 (0.17 g, 0.913 mmol) was added to the above reaction mixture and stirred at room temperature for 16 h. The reaction mixture was diluted with water (20 mL) and extracted with EtOAc (3×25 mL). The combined organic layer was washed with 10% NaHCO₃ solution (20 mL), water (25 mL), brine (25 mL), dried (Na₂SO₄), filtered and concentrated to afford the title compound (0.31 g) as a dark blue oil. LC-MS retention time=2.10 min; m/z=422.2 [M+H]⁺. Column: KINETIX XB-C18, 75×3 mm, 2.6 μm; Flow rate: 1 mL/min; Mobile Phase A: 10 mM HCOONH₄ in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm.

### Intermediate BB-22

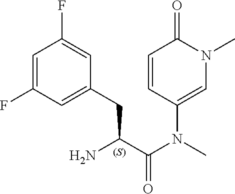

To a stirred solution of Intermediate BB-21 (0.3 g, 0.712 mmol) in dioxane (1mL) at 0° C. was added 4M HCl in dioxane (3 mL, 0.712 mmol) and the reaction mixture was stirred at room temperature for 3 h. The reaction mixture was concentrated to dryness; and the residue was triturated with diethyl ether (3×50 mL). The resulting solid was allowed to settle and the supernatant was decanted to afford the title compound (0.21 g) as a brown solid. LC-MS retention time=0.92 min; m/z=322.2 [M+H]⁺. Column: KINETIX XB-C18, 75×3 mm, 2.6 μm; Flow rate: 1 mL/min; Mobile Phase A: 10 mM HCOONH₄ in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm.

### Intermediate BB-23

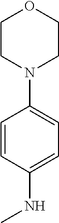

To a solution of 4-morpholinoaniline (1.5 g, 8.42 mmol) in methanol (10 mL) was added sodium methoxide (2.2 mL, 10.10 mmol), paraformaldehyde (0.278 g, 9.26 mmol) and the reaction mixture was at room temperature for 10 h. To this stirred reaction mixture was added sodium borohydride (0.48 g, 12.62 mmol) and stirred further at room temperature for 2 h. The reaction mixture was diluted with aqueous saturated solution of NH₄Cl (50 mL) and extracted with EtOAc (2×50 mL). The combined organic layer was washed with brine (75 mL), dried (Na₂SO₄), filtered and concentrated to afford the title compound (1.3 g) as a yellow solid. LC-MS retention time=0.99 min; m/z=193.2 [M+H]⁺. Column: KINETIX XB-C18, 75×3 mm, 2.6 win; Flow rate: 1 mL/min; Mobile Phase A: 10 mM HCOONH₄ in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm. ¹H NMR (400 MHz, DMSO-d₆) δ 6.77 (d, J=9.04 Hz, 2H), 6.48 (d, J=9.04 Hz, 2H), 5.13 (br. s., 1H), 3.70 (t, J=4.8 Hz, 4H), 2.89 (t, J=4.8 Hz, 4H), 2.62 (s, 3H).

### Intermediate BB-24

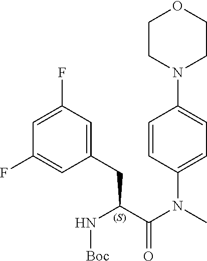

To a stirred solution of (S)-2-((tert-butoxycarbonyl)amino)-3-(3,5-difluorophenyl)propanoic acid (2.59 g, 8.58 mmol) in DMF (20 mL) was added Intermediate BB-23 (1.5 g, 7.8 mmol), HATU (4.45 g, 11.7 mmol) followed by DIPEA (6.81 mL, 39.0 mmol) and the reaction mixture was stirred at room temperature for 3 h. The reaction mixture was diluted with water (25 mL), extracted with EtOAc (2×25 mL) and the combined organic layer was washed with brine (50 mL), dried (Na₂SO₄), filtered and concentrated to afford the title compound (2.5 g) as a brown color solid. The crude was taken to next reaction without any further purification. LC-MS retention time=2.8 min; m/z=476.2 [M+H]⁺. Column: KINETIX XB-C18, 75×3 mm, 2.6 μm; Flow rate: 1 mL/min; Mobile Phase A: 10 mM HCOONH₄ in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm.

### Intermediate BB-25

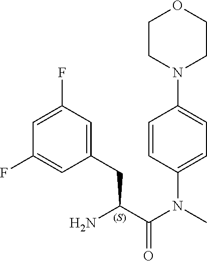

HCl in dioxane (4M solution, 13.0 mL, 52.6 mmol) was added to the Intermediate BB-24 (2.5 g, 5.3 mmol) and the reaction mixture stirred at room temperature for 2 h. The crude reaction mixture was concentrated to dryness; the crude product was basified with saturated aq. NaHCO₃ (25 mL) solution and extracted with EtOAc (2×25 mL). The combined organic layer was dried (Na₂SO₄), filtered and concentrated to afford the title compound (1.2 g) as pale red liquid. LC-MS retention time=1.93 min; m/z=376.2 [M+H]⁺. Column: KINETIX XB-C18, 75×3 mm, 2.6 win; Flow rate: 1 mL/min; Mobile Phase A: 10 mM HCOONH₄ in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm.

### Intermediate BB-26

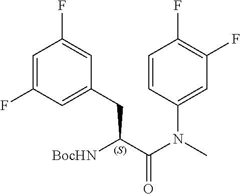

To a stirred solution of (S)-2-((tert-butoxycarbonyl)amino)-3-(3,5-difluorophenyl)propanoic acid (750 mg, 2.49 mmol), HATU (947 mg, 2.49 mmol) and DIPEA (0.435 mL, 2.49 mmol) in DMF (7.5 mL) was added 3,4-difluoro-N-methylaniline (356 mg, 2.49 mmol) and the reaction mixture was stirred at room temperature for 16 h. The reaction mass quenched into water (50 mL) and extracted with EtOAc (2×30 mL). The combined organic layer was washed with water (30 mL), brine (30 mL), dried (Na₂SO₄), filtered and concentrated. The crude product was purified by combiflash chromatography (40 g Redisep® SiO₂ column, eluting with 40-45% EtOAc in n-hexanes) to afford the title compound (860 mg) as a colorless liquid. LC-MS retention time=4.37 min; m/z=427.0 [M+H]⁺. Column: KINETIX XB-C18, 75×3 mm, 2.6 μm; Flow rate: 1 mL/min; Mobile Phase A: 10 mM HCOONH₄ in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm. ¹H NMR (300 MHz, DMSO-d₆) δ 7.74-7.38 (m, 2H), 7.21 (d, J=7.93 Hz, 1H), 7.03 (t, J=9.3 Hz, 1H), 6.63 (br. s., 2H), 4.18 (br. s., 1H), 3.05 (s, 3H), 2.86 (d, J=9.44 Hz, 2H), 1.36 (s, 9H).

### Intermediate BB-27

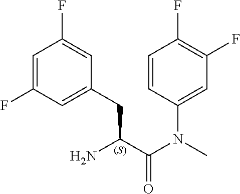

To a Intermediate BB-26 (860 mg, 2.02 mmol) was added a solution of 4 M HCl (10 mL, 40 mmol) in dioxane and stirred at room temperature for 1 h. The crude reaction mixture was concentrated to dryness; the crude product was basified with saturated aqueous solution of Na₂CO₃ and extracted with EtOAc (2×30 mL). The combined organic layer were washed with water (40 mL), brine (40 mL), dried (Na₂SO₄), filtered and concentrated to afford the title compound (525 mg) as colorless oil. LC-MS retention time=1.96 min; m/z=327.2 [M+H]⁺. Column: KINETIX XB-C18, 75×3 mm, 2.6 μm; Flow rate: 1 mL/min; Mobile Phase A: 10 mM HCOONH₄ in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm. ¹H NMR (300 MHZ, DMSO-d₆) δ 7.49 (dd, J=19.2, 9.0 Hz, 1H), 7.32-7.23 (m, 1H), 7.09-6.99 (m, 2H), 6.66 (br. s., 1H), 3.41-3.36 (m, 1H), 3.10 (br. s., 3H), 2.73 (br. s., 1H), 2.56 (br. s., 1H), 1.85 (br. s., 2H).

### Intermediate BB-28.1

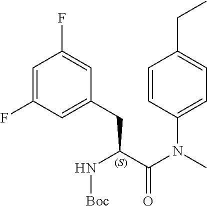

To a stirred solution of (S)-2-((tert-butoxycarbonyl)amino)-3-(3,5-difluorophenyl)propanoic acid (4.46 g, 14.8 mmol) in DMF (100 mL) was added HATU (8.44 g, 22.2 mmol), TEA (4.12 mL, 29.6 mmol) and the reaction mixture was stirred for 30 min. N-methyl-4-ethylaniline (2.00 g, 14.8 mmol) was added to the above reaction mixture and stirred at room temperature for 16 h. The reaction mixture was then diluted with water (50 mL) and extracted with EtOAc (3×50 mL). The combined organic layer was washed with 10% aqueous NaHCO₃ solution (50 mL), brine (50 mL), dried (Na₂SO₄), filtered and concentrated. The crude product was purified by combiflash chromatography (40 g Redisep® SiO₂ column, eluting with 0-15% EtOAc in hexanes) to afford the title compound (2.5 g) as an off white solid. LC-MS retention time=3.7 min; m/z=419.2 [M+H]⁺. Column: KINETIX XB-C18, 75×3 mm, 2.6 μm; Flow rate: 1 mL/min; Mobile Phase A: 10 mM HCOONH₄ in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm.

### Intermediate BB-28.2

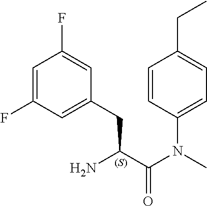

To a stirred solution of Intermediate BB-28.1 (4.0 g, 9.6 mmol) in DCM (25 mL) was added HCl in dioxane (4M, 10 mL, 38.2 mmol) and the reaction mixture was stirred at room temperature for 4 h. The reaction mixture was concentrated to dryness; the residue was triturated with hexane (2×25 mL) to afford the title compound (3.0 g) as an off white solid. LC-MS retention time=1.03 min; m/z=319.2 [M+H]⁺. Column: Acquity BEH C8 (2.1×50 mm) 1.7μ: Flow rate: 0.8 mL/min; Mobile Phase A: 5 mM NH₄OAc in water: ACN (95:5); Mobile Phase B: 5 mM NH₄OAc in water: ACN (5:95); 5% B to 99% B over 1.1 minutes and then hold a 0.6 min. at 95% B of flow rate 0.8 mL/min; Detection: UV at 220 nm.

### Intermediate BB-29.1

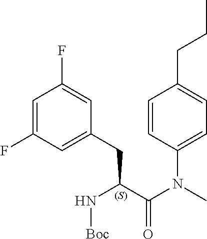

To a stirred solution of (S)-2-((tert-butoxycarbonyl)amino)-3-(3,5-difluorophenyl)propanoic acid (200 mg, 0.66 mmol) in DMF (5 mL) was added HATU (380 mg, 0.1 mmol), DIPEA (0.39 mL, 1.99 mmol) and the reaction mixture was stirred for 30 min. N-methyl-4-propylaniline (120 mg, 0.8 mmol) was added to the above reaction mixture and stirred at room temperature for 16 h. The reaction mixture was then diluted with water (20 mL) and extracted with EtOAc (3×25 mL). The combined organic layer was washed with 10% aqueous NaHCO₃ solution (25 mL), water (25 mL), brine (25 mL) dried (Na₂SO₄), filtered, concentrated and the crude product was purified by combiflash chromatography (24 g Redisep® SiO₂ column, eluting with 0-15% EtOAc in hexanes) to afford the title compound (0.25 g) as an off white solid. LC-MS retention time=1.33 min; m/z=377.2 [M-isobutylene+H]⁺. Column: Acquity BEH C8 (2.1×50 mm) 1.7μ: Flow rate: 0.8 mL/min; Mobile Phase A: 5 mM NH₄OAc in water (pH=3.5): ACN (95:5); Mobile Phase B: 5 mM NH₄OAc in water: ACN (5:95); 5% B to 95% B over 1.1 minutes and then hold a 0.6 min. at 95% B of flow rate 0.8 mL/min; Detection: UV at 220 nm. ¹H NMR (300 MHZ, DMSO-d₆) δ 7.35 (d, J=8.4 Hz, 2H), 7.31 (d, J=9.0 Hz, 2H), 7.13 (d, J=8.3 Hz, 1H), 6.99 (t, J=9.0 Hz, 1H), 6.34 (d, J=7.2 Hz, 2H), 4.17 (m, 1H), 3.17 (s, 3H), 2.77-2.58 (m, 4H), 1.70-1.59 (m, 2H), 1.30 (s, 9H), 0.92 (t, J=7.4 Hz, 3H).

### Intermediate BB-29.2

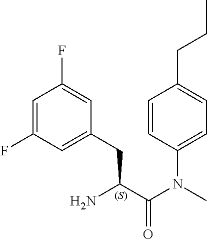

To a stirred solution of Intermediate BB-29.1 (250 mg, 0.58 mmol) in DCM (10 mL) was added HCl in dioxane (4M, 457 μL, 1.83 mmol) and the reaction mixture was stirred at room temperature for 4 h. The reaction mixture was concentrated to dryness; the residue was triturated with hexane (2×25 mL) to afford the title compound (0.2 g) as an off white solid. LC-MS retention time=2.93 min; m/z=333.2 [M+H]⁺. Column: KINETIX XB-C18, 75×3 mm, 2.6 μm; Flow rate: 1 mL/min; Mobile Phase A: 10 mM HCOONH₄ in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm.

### Intermediate BB-30.1

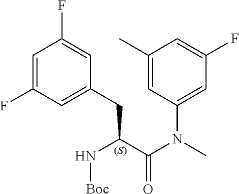

To a stirred solution of (S)-2-((tert-butoxycarbonyl)amino)-3-(3,5-difluorophenyl)propanoic acid (200 mg, 0.66 mmol) in DCM (5 mL) was added 3-fluoro-N,5-dimethylaniline (139 mg, 0.996 mmol), N-ethoxycarbonyl-2-ethoxy-1,2-dihydroquinoline (226 mg, 0.913 mmol) and the reaction mixture was stirred at room temperature for 16 h. The reaction mixture was concentrated and the crude product was purified by combiflash chromatography (40 g Redisep® SiO₂ column, eluting with 20% EtOAc in hexanes) to afford the title compound (0.25 g) as an off white solid. LC-MS retention time=3.18 min; m/z=367.2 [M-isobutylene+H]⁺. Column: KINETIX XB-C18, 75×3 mm, 2.6 μm; Flow rate: 1 mL/min; Mobile Phase A: 5 mM NH₄OAc in 98% Water/2% ACN; Mobile Phase B: 5 mM NH₄OAc in 2% Water/98% ACN; 20% B to 100% B over 4 min, then hold for 0.6 min at 100% B with flow rate of 1.5 mL/min; Detection: UV at 220 nm. ¹H NMR (400 MHZ, CDCl₃) δ 6.87 (d, J=8.8 Hz, 1H), 6.69 (tt, J=9.0, 2.2 Hz, 1H), 6.50 (br. s., 2H), 6.40 (br. s., 2H), 5.17 (br. s., 1H), 4.50 (d, J=5.8 Hz, 1H), 3.17 (s, 3H), 2.88 (dd, J=13.0, 8.3 Hz, 1H), 2.77-2.68 (m, 1H), 2.32 (s, 3H), 1.40 (br. s., 9H).

### Intermediate BB-30.2

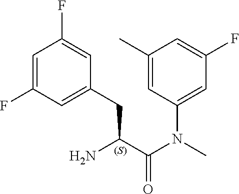

To a solution of Intermediate BB-30.1 (250 mg, 0.59 mmol) in DCM (10 mL) was added HCl in dioxane (4M, 0.15 mL, 0.59 mmol) and the reaction mixture was stirred at room temperature for 4 h. The reaction mixture was concentrated to dryness; the residue was triturated with n-hexane (2×25 mL) to afford the title compound (0.2 g) as an off white solid. LC-MS retention time=2.83 min; m/z=323.2 [M+H]⁺. Column: KINETIX XB-C18, 75×3 mm, 2.6 μm; Flow rate: 1 mL/min; Mobile Phase A: 5 mM NH₄OAc in 98% Water/2% ACN; Mobile Phase B: 5 mM NH₄OAc in 2% Water/98% ACN; 20% B to 100% B over 4 min, then hold for 0.6 min at 100% B with flow rate of 1.5 mL/min; Detection: UV at 220 nm.

### Intermediate BB-31.1

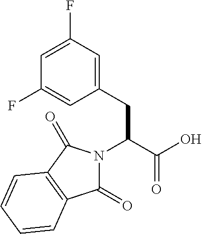

A mixture of (S)-2-amino-3-(3,5-difluorophenyl)propanoic acid (1.72 g, 8.55 mmol) and isobenzofuran-1,3-dione (1.27 g, 8.55 mmol) in DMF (12 mL) in a microwave vial (20 mL) was heated at 155° C. for 1 h in a microwave reactor. The crude mixture was poured into water (50 mL) and stirred for 30 min. The solids were collected by filtration, washed with water and dried under high vacuum overnight to afford the title compound (2.3 g) as light brown solid. LC-MS retention time=3.57 min; m/z=354.07 [M+Na]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (400 MHZ, DMSO-d₆) 8 13.45 (br. s, 1H), 7.87 (s, 4H), 7.02-6.94 (m, 3H), 5.23 (d, J=4.8 Hz, 0.5H), 5.20 (d, J=4.8 Hz, 0.5H), 3.53 (d, J=4.5 Hz, 0.5H), 3.50 (d, J=4.5 Hz, 0.5 H), 3.35 (m, 1H, overlapped with water peak).

### Intermediate BB-31.2

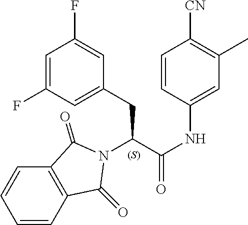

To a stirred solution of Intermediate BB-31.1 (0.400 g, 1.21 mmol), 4-amino-2-methylbenzonitrile (0.191 g, 1.45 mmol) and pyridine (0.586 mL, 7.24 mmol) in DCM (8 mL) was added POCl₃ (0.338 mL, 3.62 mmol) at 0° C. and the reaction mixture was stirred at room temperature for 16 h. The reaction mixture was diluted with 10% aqueous solution of NaHCO₃ (25 mL) and extracted with DCM (2×25 mL). The combined organic layer was dried (Na₂SO₄), filtered, concentrated and the crude product was washed with mixture of diethyl ether and DCM (7:3) and dried under vacuum to afford the title compound (0.35 g) as an off white solid. LC-MS retention time=3.02 min; m/z=446.2 [M+H]⁺. Column: KINETIX XB-C18, 75×3 mm, 2.6 μm; Flow rate: 1 mL/min; Mobile Phase A: 10 mM HCOONH₄ in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm. ¹H NMR (300 MHz, DMSO-d₆) δ 10.24 (s, 1H), 7.87 (s, 4H), 7.72 (d, J=9.3 Hz, 1H), 7.64 (d, J=7.5 Hz, 2H), 7.03-6.82 (m, 3H), 5.27 (dd, J=10.76, 4.72 Hz, 1H), 3.59 (dd, J=13.79, 4.72 Hz, 1H), 3.28 (dd, J=13.79, 4.72 Hz, 1H), 2.42 (s, 3 H).

### Intermediate BB-31.3

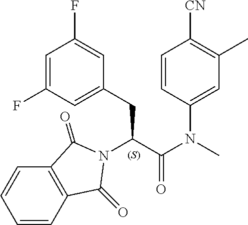

To a stirred solution of Intermediate BB-31.2 (0.35 g, 0.79 mmol) in DMF (8 mL) was added portion wise NaH (0.063 g, 60% in mineral oil, 1.6 mmol) at 0° C. and stirred for 10 min. Methyl iodide (0.197 mL, 3.14 mmol) was added at 0° C. and stirred further at room temperature for 16 h. The reaction mixture was diluted with ice cold saturated aqueous solution of NH₄Cl (50 mL) and extracted with EtOAc (2×50 mL). The combined organic layer was washed with brine (75 mL), dried (Na₂SO₄), filtered, concentrated and the crude product was purified by combiflash chromatography (24 g Redisep® SiO₂ column, eluting with 25% EtOAc in n-hexanes) to afford the title compound (0.25 g) as a pale yellow solid. LC-MS retention time=3.31 min; m/z=460.2 [M+H]⁺. Column: KINETIX XB-C18, 75×3 mm, 2.6 μm; Flow rate: 1 mL/min; Mobile Phase A: 10 mM HCOONH₄ in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm. ¹H NMR (300 MHz, DMSO-d₆) δ 7.84-7.76 (m, 2H), 7.67 (dd, J=5.4, 3.02 Hz, 2H), 7.50 (d, J=7.6 Hz, 1H), 7.22 (d, J=9.4 Hz, 1H), 7.12 (br. s., 1H), 6.94 (t, J=9.4 Hz, 1H), 6.82 (d, J=6.8 Hz, 2H), 5.28 (dd, J=10.4, 5.2 Hz, 1H), 3.47 (dd, J=13.8, 4.8 Hz, 1H), 3.18 (s, 3H), 3.08 (dd, J=13.8, 10.4 Hz, 1H), 1.99 (s, 3 H).

### Intermediate BB-31.4

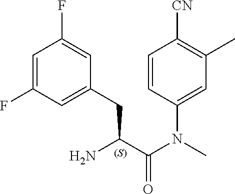

To a stirred solution of Intermediate BB-31.3 (0.17 g, 0.37 mmol) in ethanol (4 mL) in a sealed tube was added 40% aqueous solution of methylamine (0.144 g, 1.85 mmol) and the resultant reaction mixture was stirred at 65° C. for 16 h. The reaction mixture was cooled to room temperature; ethanol was removed under reduced pressure and the residue was diluted with water (25 mL) and extracted with EtOAc (2×25 mL). The combined organic layer was dried (Na₂SO₄), filtered, concentrated and the crude product was purified by combiflash chromatography (12 g Redisep® SiO₂ column, eluting with 3% MeOH in chloroform) to afford the title compound (100 mg) as a yellow solid. LC-MS retention time=0.98 min; m/z=330.3 [M+H]⁺. Column: Acquity BEH C8 (2.1×50 mm) 1.7μ: Flow rate: 0.5 mL/min; Mobile Phase A: 5 mM NH₄OAc in water: ACN (95:5); Mobile Phase B: 5 mM NH₄OAc in water: ACN (5:95); 20% B to 90% B over 1.1 minutes and then hold a 0.6 min. at 90% B of flow rate 0.5 mL/min; Detection: UV at 220 nm.

### Intermediate BB-32.1

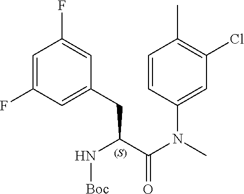

To a stirred solution of (S)-2-((tert-butoxycarbonyl)amino)-3-(3,5-difluorophenyl)propanoic acid (250 mg, 0.83 mmol) in DCM (5 mL) was added 3-fluoro-N,5-dimethylaniline (155 mg, 0.996 mmol), N-ethoxycarbonyl-2-ethoxy-1,2-dihydroquinoline (226 mg, 0.913 mmol) and the reaction mixture was stirred at room temperature for 16 h. The reaction mixture was concentrated and the crude product was purified by combiflash chromatography (40 g Redisep® SiO₂ column, eluting with 20% EtOAc in n-hexanes) to afford the title compound (0.25 g) as an off white solid. LC-MS retention time=1.46 min; m/z=439.2 [M+H]⁺. Column: Acquity BEH C8 (2.1×50 mm) 1.7μ: Flow rate: 0.5 mL/min; Mobile Phase A: 5 mM NH₄OAc in water: ACN (95:5); Mobile Phase B: 5 mM NH₄OAc in water: ACN (5:95); 20% B to 90% B over 1.1 minutes and then hold a 0.6 min. at 90% B of flow rate of 0.5 mL/min; Detection: UV at 220 nm. ¹H NMR (400 MHZ, CDCl₃) δ 7.24 (d, J=8.3 Hz, 1H), 6.80-6.66 (m, 3H), 6.50 (d, J=6.5 Hz, 2H), 5.19 (d, J=8.0 Hz, 1H), 4.52-4.40 (m, 1H), 3.19 (s, 3H), 2.87 (dd, J=13.2, 8.0 Hz, 1H), 2.72 (dd, J=13.2, 6.0 Hz, 1H), 2.39 (s, 3H), 1.41 (br. s., 9H).

### Intermediate BB-32.2

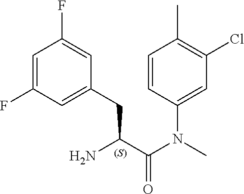

To a stirred solution of Intermediate BB-32.1 (250 mg, 0.57 mmol) in DCM (10 mL) was added HCl in dioxane (0.15 mL, 0.57 mmol, 4M in dioxane) and stirred at room temperature for 4 h. The reaction mixture was concentrated to dryness; the residue was triturated with n-hexane (2×25 mL) to afford the title compound (0.210 g) as an off white solid. LC-MS retention time=3.13 min; m/z=339.2 [M+H]⁺. Column: KINETIX XB-C18, 75×3 mm, 2.6 μ; Flow rate: 1 mL/min; Mobile Phase A: 10 mM HCOONH₄ in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm. ¹H NMR (300 MHZ, DMSO-d₆) δ 8.47 (br. s., 2H), 7.41 (d, J=7.9 Hz, 1H), 7.23-7.01 (m, 3H), 6.63 (d, J=6.8 Hz, 2H), 3.98 (br. s., 1H), 3.14 (s, 3H), 2.95-2.89 (m, 2H), 2.34 (s, 3H).

### Intermediate BB-33.1

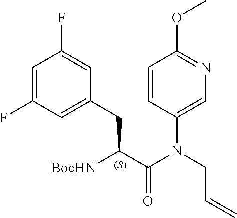

To a stirred solution of (S)-2-((tert-butoxycarbonyl) amino)-3-(3, 5-difluorophenyl) propanoic acid (300 mg, 0.99 mmol), N-allyl-6-methoxypyridin-3-amine (164 mg, 0.99 mmol) and pyridine (0.24 mL, 3.0 mmol) in DCM (5 mL) was added drop wise POCl₃ (0.09 mL, 0.99 mmol) at 0° C. and the reaction mixture was allowed to warm to RT and stirred for 3 h. The reaction mixture was diluted with DCM (˜20 mL), washed with water (20 mL), brine (20 mL), dried (Na₂SO₄), filtered, concentrated and the crude product was purified by combiflash chromatography (40 g Redisep® SiO₂ column, eluting with 10-15% EtOAc in hexanes) to afford the title compound (110 mg) as a colorless liquid. LC-MS retention time=3.35 min; m/z=448.2 [M+H]⁺. Column: KINETIX C18, 75×3 mm, 2.6 μm; Flow rate: 1 mL/min; Mobile Phase A: 10 mM HCO₂NH₄ in 98% Water/2% ACN; Mobile Phase B: 10 mM HCO₂NH₄ in 2% Water/98% ACN; 20% B to 100% B over 4 min, then hold for 0.6 min at 100% B with flow rate of 1.5 mL/min; Detection: UV at 254 nm. ¹H NMR (400 MHz, CDCl₃) δ 7.67 (br s, 1H), 7.00 (br s, 1H), 6.74-6.66 (m, 2H), 6.52 (d, J=6.5 Hz, 2H), 5.76 (ddt, J=16.9, 10.2, 6.5 Hz, 1H), 5.18-5.12 (m, 2H), 5.30 (dd, J=16.8, 1.2 Hz, 1H), 4.37 (d, J=7.5 Hz, 1H), 4.19 (m, 2H), 3.95 (s, 3H), 2.90 (dd, J=13.1, 7.5 Hz, 1H), 2.73 (dd, J=13.3, 6.3 Hz, 1H), 1.39 (s, 9H).

### Intermediate BB-33.2

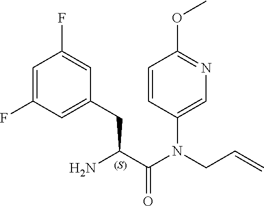

HCl in dioxane (4 M solution, 25.8 mL, 103 mmol) was added to Intermediate BB-33.1 (110 mg, 0.25 mmol) and stirred at RT for 16 h. The reaction mixture was concentrated to dryness, the residue was treated with saturated aqueous solution of NaHCO₃ and extracted with DCM (3×20 mL). The combined organic component was washed with water (20 mL), brine (20 mL), dried (Na₂SO₄), filtered and concentrated to afford the title compound (82 mg) as an off-white solid. LC-MS retention time=2.02 min; m/z=348.2 [M+H]⁺. Column: KINETIX C18, 75×3 mm, 2.6 μm; Flow rate: 1 mL/min; Mobile Phase A: 10 mM HCO₂NH₄in 98% Water/2% ACN; Mobile Phase B: 10 mM HCO₂NH₄ in 2% Water/98% ACN; 20% B to 100% B over 4 min, then hold for 0.6 min at 100% B with flow rate of 1.5 mL/min; Detection: UV at 220 nm.

### Intermediate BB-34.1

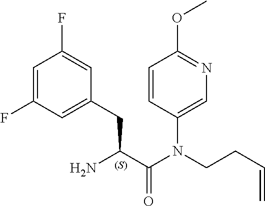

K₂CO₃ (7.35 g, 53.2 mmol) was added to stirred solution of 5-bromo-2-methoxypyridine (5.00 g, 26.6 mmol) in DMSO (50 mL) followed by L-proline (0.306 g, 2.66 mmol), but-3-en-1-amine (3.68 mL, 39.9 mmol) and CuI (1.013 g, 5.32 mmol). The reaction mixture was stirred at 80° for 12 h, filtered through Celite and partitioned between water and EtOAC. The organic layer was washed with water (2×), and concentrated in vacuo, and the resultant residue was purified via Biotage (20% EtOAC/hexanes to afford N-(but-3-en-1-yl)-6-methoxypyridin-3-amine (2.7 g).

POCl₃ (0.351 mL, 3.77 mmol) and pyridine (0.915 mL, 11.3 mmol) were added to stirred solution of (S)-2-((tert-butoxycarbonyl)amino)-3-phenylpropanoic acid (1.00 g, 3.77 mmol) in CH₂Cl₂ (5.0 mL). Then N-(but-3-en-1-yl)-6-methoxypyridin-3-amine (0.672 g, 3.77 mmol) was added to reaction mixture and stirred for 2 h at 0°. The reaction mixture was poured into a bicarbonate solution, and then carefully extracted with EtOAC. The organic layer was washed with water (2×), concentrated in vacuo and the resultant residue was purified with Biotage (20% EtOAC/hexanes) to afford (S)-tert-butyl (1-(but-3-en-1-yl(6-methoxypyridin-3-yl)amino)-1-oxo-3-phenylpropan-2-yl)carbamate (0.60 g).

4 N HCl in 1,4-Dioxane (3.5 mL, 14 mmol) was added to a stirred solution of (S)-tert-butyl (1-(but-3-en-1-yl(6-methoxypyridin-3-yl)amino)-1-oxo-3-phenylpropan-2-yl)carbamate (0.60 g, 1.410 mmol) in 1,4-Dioxane (1.0 mL) at 0° C. The reaction mixture was stirred for at room temperature for 14 h, poured into a bicarbonate solution, then carefully extracted with EtOAC. The organic layer was washed with water and concentrated in vacuo to afford Intermediate 34.1, which was used as crude in a subsequent step.

Note: Unless noted otherwise, the following Examples were purified using preparative HPLC, reverse phase C18 columns, eluting with either MeOH/water or acetonitrile/water buffered with ammonium acetate.

### Example 1

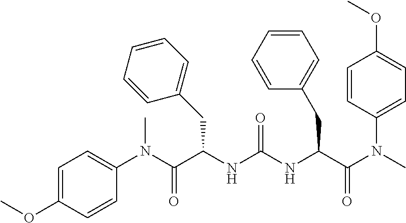 [SMILES: COc1ccc(N(C)C(=O)[C@H](Cc2ccccc2)NC(=O)N[C@@H](Cc2ccccc2)C(=O)N(C)c2ccc(OC)cc2)cc1]

CDI (33.4 mg, 0.206 mmol) and DIPEA (0.078 mL, 0.45 mmol) were added to a stirred solution of Intermediate 4, HCl (60 mg, 0.187 mmol) in DCM (2 mL) and the reaction mixture was stirred at rt overnight. The reaction was conc. to dryness, treated with pyridin-2-amine (21.12 mg, 0.224 mmol) and toluene (3 mL) and heated at reflux for 18 h and then stirred at rt for 3 days. The reaction mixture was concentrated to dryness and portioned between IN HCl (aq) and EtOAc and the organic component was washed with brine, dried (MgSO₄), filtered and concentrated. The residue was dissolved into MeOH, filtered and purified by preparative HPLC to yield the title compound (13.9 mg). LC-MS retention time=2.09 min; m/z=595.3 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, DMSO-d₆) δ ppm 7.25-7.15 (m, 6H), 7.01 (d, J=7.7 Hz, 4H), 6.95-6.91 (m, 4H), 6.85 (d, J=6.6 Hz, 4H), 6.35 (d, J=8.4 Hz, 2H), 4.27-4.20 (m, 2H), 3.79 (s, 6H), 3.06 (s, 6H), 2.79-2.72 (m, 2H), 2.48 (d, J=8.4 Hz, 2H).

### Example 2

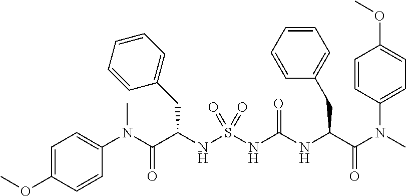 [SMILES: COc1ccc(N(C)C(=O)[C@H](Cc2ccccc2)NC(=O)NS(=O)(=O)N[C@@H](Cc2ccccc2)C(=O)N(C)c2ccc(OC)cc2)cc1]

A solution of an HCl salt of Intermediate 4 (180 mg, 0.505 mmol) in DCM (1 mL) was added dropwise at 0° C. to a stirred solution of sulfurisocyanatidic chloride (0.062 mL, 0.71 mmol) in DCM (1 mL) and the reaction mixture was stirred at 0° C. for 1 h. TEA (0.225 mL, 1.62 mmol) was then added and the reaction mixture was stirred at 0° C. for 3 min. The reaction mixture was taken up in a syringe and ˜ 3/10 of the crude solution (˜1.2 mL) was added to a stirred solution of an HCl salt of Intermediate 4 (60 mg, 0.17 mmol) in DCM (1 mL) and the reaction mixture was stirred at rt overnight. The reaction mixture was concentrated and the residue was dissolved into MeOH, filtered and purified by preparative HPLC to yield the title compound (21.6 mg). LC-MS retention time=1.90 min; m/z=674.6[M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, DMSO-d₆) δ ppm 7.23-7.14 (m, 6H), 7.10-6.76 (m, 12H), 6.47 (d, J=8.1 Hz, 1H), 4.42 (q, J=7.2 Hz, 1H), 4.09 (t, J=6.8 Hz, 1H), 3.79 (s, 3H), 3.76 (s, 3H), 3.08 (s, 3H), 3.00 (s, 3H), 2.83-2.73 (m, 2H), 2.64-2.55 (m, 2H).

### Example 3

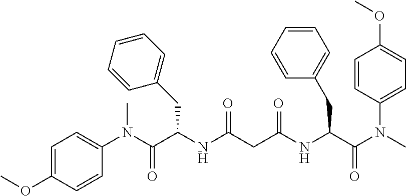 [SMILES: COc1ccc(N(C)C(=O)[C@H](Cc2ccccc2)NC(=O)CC(=O)N[C@@H](Cc2ccccc2)C(=O)N(C)c2ccc(OC)cc2)cc1]

A solution of POCl₃ (0.018 mL, 0.20 mmol) in pyridine (0.5 mL) was added to a solution of an HCl salt of Intermediate 4 (60 mg, 0.19 mmol) and malonic acid (9.7 mg, 0.094 mmol) in pyridine (1 mL) and DIPEA (0.065 mL, 0.374 mmol) and the reaction mixture was stirred at rt for 16 h. The reaction was concentrated and the residue was dissolved in MeOH and then purified via preparative LC/MS (Column: waters xbridge C18, 19×200 mm, 5-μm particles; Mobile Phase A: 5:95 acetonitrile: water with 0.1% TFA; Mobile Phase B: 95:5 acetonitrile: water with 0.1% TFA; Gradient: 45-85% B over 15 minutes, then a 5-minute hold at 100% B; Flow: 20 mL/min. Fractions containing the title compound were combined and dried via centrifugal evaporation) to yield the title compound (19.7 mg). LC-MS retention time=1.84 min; m/z=637.1 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=1.0 mL/min. Start % B=0. Final % B=100. Gradient Time=3 minutes, then a 0.5-minute hold at 100% B. Wavelength=220).

### Example 4

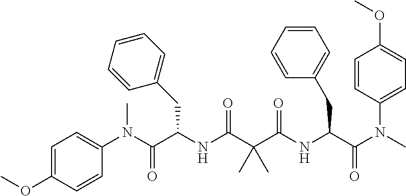 [SMILES: COc1ccc(N(C)C(=O)[C@H](Cc2ccccc2)NC(=O)C(C)(C)C(=O)N[C@@H](Cc2ccccc2)C(=O)N(C)c2ccc(OC)cc2)cc1]

DIPEA (0.049 mL, 0.28 mmol) was added to a mixture of an HCl salt of Intermediate 4 (60 mg, 0.19 mmol) and 2,2-dimethylmalonyl dichloride (16 mg, 0.090 mmol) in DCM (1 mL) and the reaction mixture was stirred at rt for 16 h. The reaction mixture was concentrated, and the residue was dissolved in MeOH and then purified via preparative LC/MS (Column: waters xbridge C18, 19×200 mm, 5-μm particles; Mobile Phase A: 5:95 acetonitrile: water with 0.1% TFA; Mobile Phase B: 95:5 acetonitrile: water with 0.1% TFA; Gradient: 50-90% B over 15 minutes, then a 5-minute hold at 100% B; Flow: 20 mL/min. Fractions containing the title compound were combined and dried via centrifugal evaporation) to yield the title compound (46 mg). LC-MS retention time=2.02 min; m/z=665.2 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=1.0 mL/min. Start % B=0. Final % B=100. Gradient Time=3 minutes, then a 0.5-minute hold at 100% B. Wavelength=220).

### Example 5

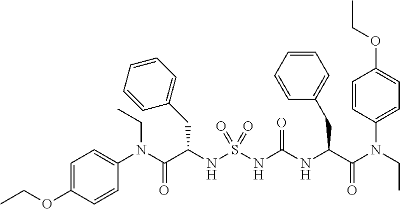 [SMILES: CCOc1ccc(N(CC)C(=O)[C@H](Cc2ccccc2)NC(=O)NS(=O)(=O)N[C@@H](Cc2ccccc2)C(=O)N(CC)c2ccc(OCC)cc2)cc1]

A solution of Intermediate 8 (88 mg, 0.28 mmol) in DCM (0.8 mL) was added to a stirred solution of sulfurisocyanatidic chloride (20 mg, 0.14 mmol) in DCM (0.8 mL) and the reaction mixture was stirred 10 min at rt. Then TEA (0.063 mL, 0.45 mmol) was added and the reaction mixture was stirred at rt overnight. The reaction was concentrated, diluted with EtOAc (˜5 mL) and washed with 1M HCl (2 mL) and brine (2 mL). The organic component was concentrated, dissolved into MeOH, filtered and purified by preparative HPLC to yield the title compound (23 mg). LC-MS retention time=2.36 min; m/z=730.6 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 6

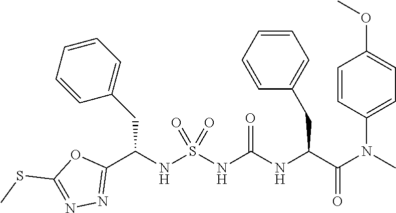 [SMILES: COc1ccc(N(C)C(=O)[C@H](Cc2ccccc2)NC(=O)NS(=O)(=O)N[C@@H](Cc2ccccc2)c2nnc(SC)o2)cc1]

To a solution of sulfurisocyanatidic chloride (140 μL, 1.6 mmol) in DCM (2.5 mL) at 0° C. was added dropwise a solution of an HCl salt of Intermediate 4 (350 mg, 1.1 mmol) in DCM (5.0 mL). The reaction mixture was stirred under nitrogen at 0° C. for 1 h and then treated with a solution of triethylamine (680 μL, 4.9 mmol) in DCM (2.5 mL) and allowed to stir for 5 min. A portion of the reaction mixture (1.0 mL, 0.11 mol) was added to a solution of (S)-1-(5-(methylthio)-1,3,4-oxadiazol-2-yl)-2-phenylethanamine (51 mg, 0.22 mmol) in DCM (0.25 mL) and the reaction was shaken at rt for 2 h. The reaction mixture was concentrated, dissolved into DMF (1 mL), transferred to an empty 6-mL SPE cartridge, rinsed with DMF (0.5 mL) and purified by preparative HPLC to yield the title compound (31.9 mg). LC-MS retention time=2.64 min; m/z=625.4 [M+H]⁺. (Column: Waters BEH C18, 2.0×50 mm, 1.7-μm particles. Solvent A=95% Water: 5% MeOH: 10 mM NH₄OAc. Solvent B=5% Water: 95% MeOH: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 7

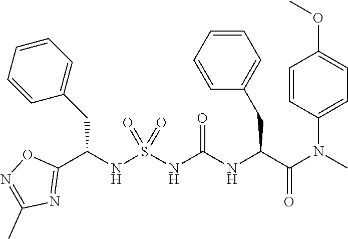 [SMILES: COc1ccc(N(C)C(=O)[C@H](Cc2ccccc2)NC(=O)NS(=O)(=O)N[C@@H](Cc2ccccc2)c2nc(C)no2)cc1]

To a solution of sulfurisocyanatidic chloride (140 μL, 1.6 mmol) in DCM (2.5 mL) at 0° C. was added dropwise a solution of an HCl salt of Intermediate 4 (350 mg, 1.1 mmol) in DCM (5.0 mL). The reaction mixture was stirred under nitrogen at 0° C. for 1 h and then treated with a solution of triethylamine (680 μL, 4.9 mmol) in DCM (2.5 mL) and allowed to stir for 5 min. A portion of the reaction mixture (1.0 mL, 0.11 mol) was added to a solution of (S)-1-(3-methyl-1,2,4-oxadiazol-5-yl)-2-phenylethanamine (44 mg, 0.22 mmol) in DCM (0.25 mL) and the reaction was shaken at rt for 2 h. The reaction mixture was concentrated, dissolved into DMF (1 mL), transferred to an empty 6-mL SPE cartridge, and rinsed with DMF (0.5 mL) and purified by preparative HPLC to yield the title compound (14.7 mg). LC-MS retention time=2.65 min; m/z=593.1 [M+H]⁺. (Column: Waters BEH C18, 2.0×50 mm, 1.7-μm particles. Solvent A=95% Water: 5% MeOH: 10 mM NH₄OAc. Solvent B=5% Water: 95% MeOH: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 8

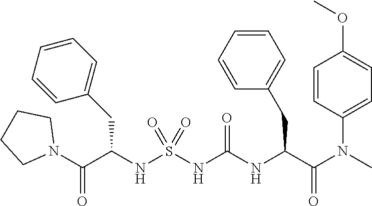 [SMILES: COc1ccc(N(C)C(=O)[C@H](Cc2ccccc2)NC(=O)NS(=O)(=O)N[C@@H](Cc2ccccc2)C(=O)N2CCCC2)cc1]

To a solution of sulfurisocyanatidic chloride (140 μL, 1.6 mmol) in DCM (2.5 mL) at 0° C. was added dropwise a solution of an HCl salt of Intermediate 4 (350 mg, 1.1 mmol) in DCM (5.0 mL). The reaction mixture was stirred under nitrogen at 0° C. for 1 h and then treated with a solution of triethylamine (680 μL, 4.9 mmol) in DCM (2.5 mL) and allowed to stir for 5 min. A portion of the reaction mixture (1.0 mL, 0.11 mol) was added to a solution of (S)-2-amino-3-phenyl-1-(pyrrolidin-1-yl)propan-1-one (48 mg, 0.22 mmol) in DCM (0.25 mL) and the reaction was shaken at rt for 2 h. The reaction mixture was concentrated, dissolved into DMF (1 mL), transferred to an empty 6-mL SPE cartridge, and rinsed with DMF (0.5 mL) and purified by preparative HPLC to yield the title compound (36.8 mg). LC-MS retention time=1.65 min; m/z=608.2 [M+H]⁺. (Column: Waters BEH C18, 2.0×50 mm, 1.7-μm particles. Solvent A=95% Water: 5% MeOH: 10 mM NH₄OAc. Solvent B=5% Water: 95% MeOH: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 9

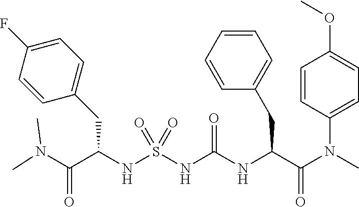 [SMILES: COc1ccc(N(C)C(=O)[C@H](Cc2ccccc2)NC(=O)NS(=O)(=O)N[C@@H](Cc2ccc(F)cc2)C(=O)N(C)C)cc1]

To a solution of sulfurisocyanatidic chloride (140 μL, 1.6 mmol) in DCM (2.5 mL) at 0° C. was added dropwise a solution of an HCl salt of Intermediate 4 (350 mg, 1.1 mmol) in DCM (5.0 mL). The reaction mixture was stirred under nitrogen at 0° C. for 1 h and then treated with a solution of triethylamine (680 μL, 4.9 mmol) in DCM (2.5 mL) and allowed to stir for 5 min. A portion of the reaction mixture (1.0 mL, 0.11 mol) was added to a solution of an HCl salt of (S)-2-amino-3-(4-fluorophenyl)-N,N-dimethylpropanamide (54 mg, 0.22 mmol) in DCM (0.25 mL) and the reaction was shaken at rt for 2 h. The reaction mixture was concentrated, dissolved into DMF (1 mL), transferred to an empty 6-mL SPE cartridge, and rinsed with DMF (0.5 mL) and purified by preparative HPLC to yield the title compound (34.6 mg). LC-MS retention time=2.63 min; m/z=600.3 [M+H]⁺. (Column: Waters BEH C18, 2.0×50 mm, 1.7-μm particles. Solvent A=95% Water: 5% MeOH: 10 mM NH₄OAc. Solvent B=5% Water: 95% MeOH: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 10

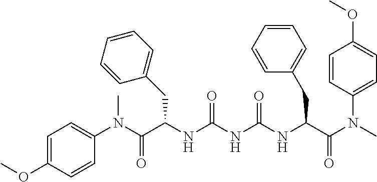 [SMILES: COc1ccc(N(C)C(=O)[C@H](Cc2ccccc2)NC(=O)NC(=O)N[C@@H](Cc2ccccc2)C(=O)N(C)c2ccc(OC)cc2)cc1]

A solution of Intermediate 4 (140 mg, 0.492 mmol) in DCM (0.8 mL) was added to a stirred solution of carbonisocyanatidic chloride (26 mg, 0.25 mmol) in DCM (0.8 mL) and the reaction mixture was stirred 10 min at rt. Then TEA (0.11 mL, 0.79 mmol) was added and the reaction mixture was stirred at rt for 3 h. The reaction mixture was concentrated and the residual material was dissolved into MeOH, filtered and purified by preparative HPLC to yield the title compound (15.5 mg). LC-MS retention time=2.09 min; m/z=638.5 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, DMSO-d₆) δ ppm 8.62 (s, 1H), 7.64 (br. s., 2H), 7.22-7.17 (m, 6H), 7.06 (br. s., 4H), 6.98 (d, J=8.8 Hz, 4H), 6.84-6.79 (m, 4H), 4.46-4.37 (m, 2H), 3.80 (s, 6H), 3.11 (s, 6H), 2.82 (dd, J=13.4, 5.3 Hz, 2H), 2.54 (dd, J=9.2, 4.4 Hz, 2H).

### Example 11

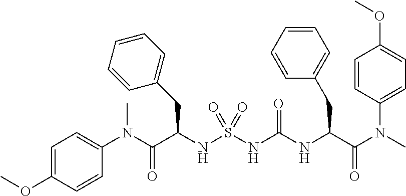 [SMILES: COc1ccc(N(C)C(=O)[C@H](Cc2ccccc2)NC(=O)NS(=O)(=O)N[C@H](Cc2ccccc2)C(=O)N(C)c2ccc(OC)cc2)cc1]

A solution of an HCl salt of Intermediate 4 (100 mg, 0.281 mmol) in DCM (1 mL) was added dropwise at 0° C. to a stirred solution of sulfurisocyanatidic chloride (0.034 mL, 0.393 mmol) in DCM (1 mL) and the reaction mixture was stirred at 0° C. for 1 h. Then TEA (0.125 mL, 0.898 mmol) was added to the reaction mixture and it was stirred at 0° C. for 3 min. Then ½ of this crude reaction solution (˜1 mL) was taken-up in a syringe and added to a stirred suspension of an HCl salt of Intermediate 5 (54.0 mg, 0.168 mmol) in DCM (1 mL) and the reaction mixture was stirred at rt overnight. The reaction mixture was concentrated and the residual material was dissolved into MeOH, filtered and purified by preparative HPLC to yield the title compound (31 mg). LC-MS retention time=2.88 min; m/z=674.6 [M+H]⁺. (Column: Waters BEH C18, 2.0×50 mm, 1.7-μm particles. Solvent A=95% Water: 5% MeOH: 10 mM NH₄OAc. Solvent B=5% Water: 95% MeOH: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 12

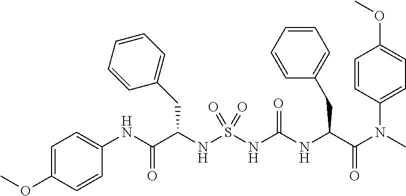 [SMILES: COc1ccc(NC(=O)[C@H](Cc2ccccc2)NS(=O)(=O)NC(=O)N[C@@H](Cc2ccccc2)C(=O)N(C)c2ccc(OC)cc2)cc1]

A solution of an HCl salt of Intermediate 4 (100 mg, 0.281 mmol) in DCM (1 mL) was added dropwise at 0° C. to a stirred solution of sulfurisocyanatidic chloride (0.034 mL, 0.39 mmol) in DCM (1 mL) and the reaction mixture was stirred at 0° C. for 1 h. Then TEA (0.125 mL, 0.898 mmol) was added to the reaction mixture and it was stirred at 0° C. for 3 min. Then ½ of this crude reaction solution (˜1 mL) was taken-up in a syringe and added to a stirred suspension of an HCl salt of Intermediate 6 (68.9 mg, 0.224 mmol) in DCM (1 mL) and TEA (2 drops) and the reaction mixture was stirred at rt overnight. The reaction mixture was concentrated and the residual material was dissolved into MeOH, filtered and purified by preparative HPLC to yield the title compound (29.7 mg). LC-MS retention time=2.76 min; m/z=660.6 [M+H]⁺. (Column: Waters BEH C18, 2.0×50 mm, 1.7-μm particles. Solvent A=95% Water: 5% MeOH: 10 mM NH₄OAc. Solvent B=5% Water: 95% MeOH: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 13

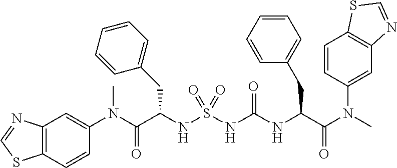 [SMILES: CN(C(=O)[C@H](Cc1ccccc1)NC(=O)NS(=O)(=O)N[C@@H](Cc1ccccc1)C(=O)N(C)c1ccc2scnc2c1)c1ccc2scnc2c1]

At 0° C., a solution of sulfurisocyanatidic chloride (6.2 μl, 0.072 mmol) in DCM (0.5 mL) was added to a solution of an HCl salt of Intermediate 18 (64.3 mg, 0.12 mmol) in DCM (0.5 mL) and TEA (0.066 mL, 0.48 mmol) and the reaction mixture was stirred at 0° C. for 16 h. The reaction mixture was concentrated, the residue was dissolved in MeOH and then purified via preparative HPLC (Column: waters xbridge C18, 19×200 mm, 5-μm particles; Mobile Phase A: 5:95 acetonitrile: water with 10 mM NH₄OAc; Mobile Phase B: 95:5 acetonitrile: water with 10 mM NH₄OAc; Gradient: 25-70% B over 35 minutes, then a 5-minute hold at 100% B; Flow: 20 mL/min. Fractions containing the title compound were combined and dried via centrifugal evaporation) to yield the title compound (4.5 mg). LC-MS retention time=1.64 min; m/z=728.5 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=1.0 mL/min. Start % B=0. Final % B=100. Gradient Time=3 minutes, then a 0.5-minute hold at 100% B. Wavelength=220).

### Example 14

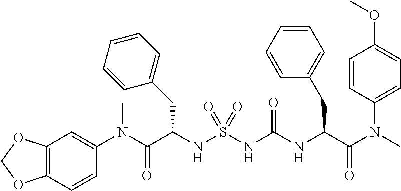 [SMILES: COc1ccc(N(C)C(=O)[C@H](Cc2ccccc2)NC(=O)NS(=O)(=O)N[C@@H](Cc2ccccc2)C(=O)N(C)c2ccc3c(c2)OCO3)cc1]

A solution of an HCl salt of Intermediate 4 (180 mg, 0.505 mmol) in DCM (2 mL) was added dropwise at 0° C. to a stirred solution of sulfurisocyanatidic chloride (0.062 mL, 0.71 mmol) in DCM (1.8 mL) and the reaction mixture was stirred at 0° C. for 1 h. Then TEA (0.225 mL, 1.62 mmol) was added and reaction mixture was stirred at 0° C. for 3 min. Then ⅕ of the total volume of the crude reaction mixture (˜0.8 mL) was added to a stirred suspension of an HCl salt of Intermediate 9 (57 mg, 0.170 mmol) in DCM (1 mL) and the reaction mixture was stirred at rt overnight. The reaction mixture was concentrated and the residual material was dissolved into MeOH, filtered and purified by preparative HPLC to yield the title compound (3.4 mg). LC-MS retention time=1.86 min; m/z=688.5 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 15

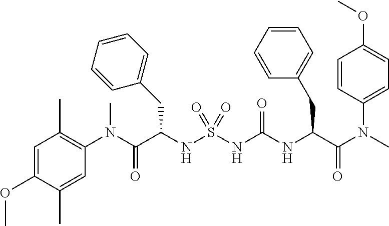 [SMILES: COc1ccc(N(C)C(=O)[C@H](Cc2ccccc2)NC(=O)NS(=O)(=O)N[C@@H](Cc2ccccc2)C(=O)N(C)c2cc(C)c(OC)cc2C)cc1]

A solution of an HCl salt of Intermediate 4 (180 mg, 0.505 mmol) in DCM (2 mL) was added dropwise at 0° C. to a stirred solution of sulfurisocyanatidic chloride (0.062 mL, 0.71 mmol) in DCM (1.8 mL) and the reaction mixture was stirred at 0° C. for 1 h. Then TEA (0.225 mL, 1.62 mmol) was added and reaction mixture was stirred at 0° C. for 3 min. Then ⅕ of the total volume of the crude reaction mixture (˜0.8 mL) was added to a stirred suspension of an HCl salt of Intermediate 10 (41 mg, 0.118 mmol) in DCM (1 mL) and the reaction mixture was stirred at rt overnight. The reaction mixture was concentrated and the residual material was dissolved into MeOH, filtered and purified by preparative HPLC to yield the title compound (10.1 mg). LC-MS retention time=2.09 min; m/z=702.6 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 16

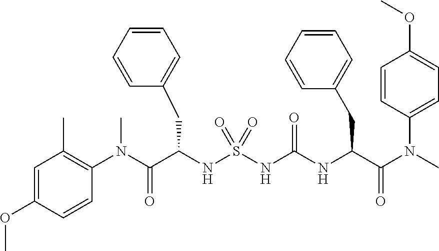 [SMILES: COc1ccc(N(C)C(=O)[C@H](Cc2ccccc2)NC(=O)NS(=O)(=O)N[C@@H](Cc2ccccc2)C(=O)N(C)c2ccc(OC)cc2C)cc1]

A solution of an HCl salt of Intermediate 4 (180 mg, 0.505 mmol) in DCM (1 mL) was added dropwise at 0° C. to a stirred solution of sulfurisocyanatidic chloride (0.062 mL, 0.71 mmol) in DCM (1 mL) and the reaction mixture was stirred at 0° C. for 1 h. TEA (0.225 mL, 1.62 mmol) was then added and the reaction mixture was stirred at 0° C. for 3 min. The reaction mixture was taken up in a syringe and ˜⅕ of the crude solution (˜0.8 mL) was added to a stirred solution of an HCl salt of Intermediate 11 (44 mg, 0.13 mmol) in DCM (1 mL) and the reaction mixture was stirred at rt overnight. The reaction mixture was concentrated and the residue was dissolved into MeOH, filtered and purified by preparative HPLC to yield the title compound (12.0 mg). LC-MS retention time=1.95 min; m/z=688.6 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 17

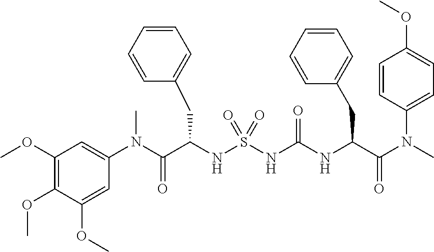 [SMILES: COc1ccc(N(C)C(=O)[C@H](Cc2ccccc2)NC(=O)NS(=O)(=O)N[C@@H](Cc2ccccc2)C(=O)N(C)c2cc(OC)c(OC)c(OC)c2)cc1]

A solution of an HCl salt of Intermediate 4 (180 mg, 0.505 mmol) in DCM (1 mL) was added dropwise at 0° C. to a stirred solution of sulfurisocyanatidic chloride (0.062 mL, 0.71 mmol) in DCM (1 mL) and the reaction mixture was stirred at 0° C. for 1 h. TEA (0.225 mL, 1.62 mmol) was then added and the reaction mixture was stirred at 0° C. for 3 min. The reaction mixture was taken up in a syringe and ˜ 1/10 of the crude solution (˜0.4 mL) was added to a stirred solution of an HCl salt of Intermediate 12 (28 mg, 0.074 mmol) in DCM (1 mL) and the reaction mixture was stirred at rt overnight. The reaction mixture was concentrated and the residue was dissolved into MeOH, filtered and purified by preparative HPLC to yield the title compound (8.7 mg). LC-MS retention time=1.84 min; m/z=734.6 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 18

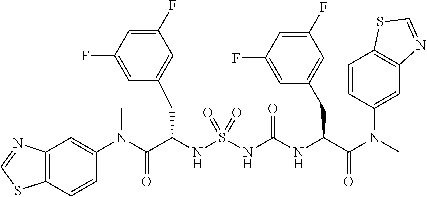 [SMILES: CN(C(=O)[C@H](Cc1cc(F)cc(F)c1)NC(=O)NS(=O)(=O)N[C@@H](Cc1cc(F)cc(F)c1)C(=O)N(C)c1ccc2scnc2c1)c1ccc2scnc2c1]

A solution of sulfurisocyanatidic chloride (6.2 μl, 0.071 mmol) in DCM (0.5 mL) was added dropwise to a solution of an HCl salt of Intermediate 20 (50 mg, 0.12 mmol) in DCM (0.5 mL) and TEA (0.066 mL, 0.48 mmol) and the reaction mixture was stirred at rt for 16 h. The reaction mixture was concentrated, the residue was dissolved in MeOH and then purified via preparative HPLC (Column: waters xbridge C18, 19×200 mm, 5-μm particles; Mobile Phase A: 5:95 acetonitrile: water with 10 mM NH₄OAc; Mobile Phase B: 95:5 acetonitrile: water with 10 mM NH₄OAc; Gradient: 25-65% B over 15 minutes, then a 5-minute hold at 100% B; Flow: 20 mL/min. Fractions containing the title compound were combined and dried via centrifugal evaporation) to yield the title compound (12.3 mg). LC-MS retention time=1.85 min; m/z=800.5 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=1.0 mL/min. Start % B=0. Final % B=100. Gradient Time=3 minutes, then a 0.5-minute hold at 100% B. Wavelength=220). ¹H NMR (500 MHZ, DMSO-d₆) δ ppm 9.57-9.33 (m, 2H), 8.31-8.10 (m, 2H), 8.06-7.82 (m, 2H), 7.57-7.25 (m, 2H), 7.08-6.87 (m, 2H), 6.58-6.23 (m, 4H), 4.54-4.06 (m, 2H), 3.27-3.02 (m, 6H), 2.85-2.73 (m, 2H), 2.69-2.58 (m, 2H).

### Example 19

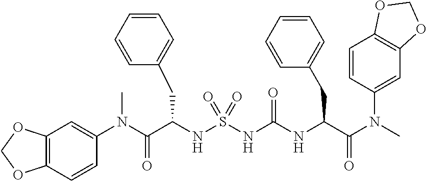 [SMILES: CN(C(=O)[C@H](Cc1ccccc1)NC(=O)NS(=O)(=O)N[C@@H](Cc1ccccc1)C(=O)N(C)c1ccc2c(c1)OCO2)c1ccc2c(c1)OCO2]

A solution of an HCl salt of Intermediate 9 (105 mg, 0.314 mmol) in DCM (1 mL) was added to a stirred solution of sulfurisocyanatidic chloride (26.6 mg, 0.188 mmol) in DCM (0.5 mL) and then the reaction mixture was treated with TEA (0.175 mL, 1.25 mmol) and stirred at rt for 1 h. Additional sulfurisocyanatidic chloride (26.6 mg, 0.188 mmol) was added and the reaction mixture was stirred overnight. The reaction mixture was concentrated and the residue was dissolved into MeOH, filtered and purified by preparative HPLC to yield the title compound (19 mg). LC-MS retention time=1.88 min; m/z=702.2 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 20

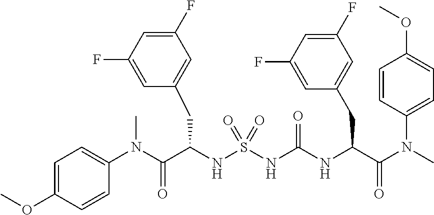 [SMILES: COc1ccc(N(C)C(=O)[C@H](Cc2cc(F)cc(F)c2)NC(=O)NS(=O)(=O)N[C@@H](Cc2cc(F)cc(F)c2)C(=O)N(C)c2ccc(OC)cc2)cc1]

A solution of an HCl salt of Intermediate 13 (105 mg, 0.294 mmol) in DCM (1 mL) was added to a stirred solution of sulfurisocyanatidic chloride (25 mg, 0.18 mmol) in DCM (0.5 mL) and then the reaction mixture was treated with TEA (0.16 mL, 1.2 mmol) and stirred at rt for 1 h. The reaction mixture was concentrated and the residue was dissolved into MeOH, filtered and purified by preparative HPLC to yield the title compound (23.9 mg). LC-MS retention time=1.99 min; m/z=746.2 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, DMSO-d₆) δ ppm 7.29-6.90 (m, 11H), 6.55 (d, J=7.0 Hz, 2H), 6.43 (d, J=7.0 Hz, 3H), 3.81 (s, 3H), 3.76 (s, 3H), 3.11 (s, 3H), 3.01 (s, 3H), 2.84-2.78 (m, 1H), 2.77-2.71 (m, 2H), 2.67-2.57 (m, 2H).

### Example 21

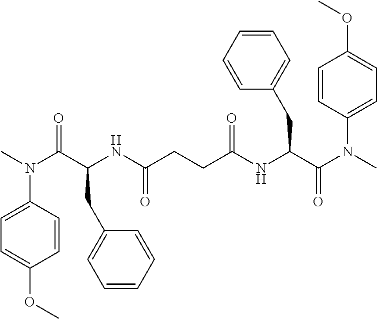 [SMILES: COc1ccc(N(C)C(=O)[C@H](Cc2ccccc2)NC(=O)CCC(=O)N[C@@H](Cc2ccccc2)C(=O)N(C)c2ccc(OC)cc2)cc1]

A solution of an HCl salt of Intermediate 4 (42 mg, 0.13 mmol) in DCM (0.5 mL) was added to a stirred solution of succinyl dichloride (9.2 mg, 0.059 mmol) in DCM (0.5 mL) at rt. Then TEA (0.05 mL, 0.36 mmol) was added and the reaction mixture was stirred at rt overnight. The reaction mixture was concentrated and the residue was dissolved into MeOH, filtered and purified by preparative HPLC to yield the title compound (25.7 mg). LC-MS retention time=1.84 min; m/z=651.5 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 22

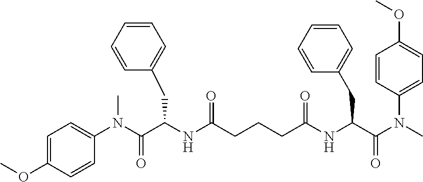 [SMILES: COc1ccc(N(C)C(=O)[C@H](Cc2ccccc2)NC(=O)CCCC(=O)N[C@@H](Cc2ccccc2)C(=O)N(C)c2ccc(OC)cc2)cc1]

A solution of an HCl salt of Intermediate 4 (42 mg, 0.13 mmol) in DCM (0.5 mL) was added to a stirred solution of glutaroyl dichloride (10 mg, 0.059 mmol) in DCM (0.5 mL) at rt. Then TEA (0.05 mL, 0.36 mmol) was added and the reaction mixture was stirred at rt for 1 h. The reaction mixture was concentrated and the residue was dissolved into MeOH, filtered and purified by preparative HPLC to yield the title compound (22.6 mg). LC-MS retention time=1.98 min; m/z=665.7 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 23

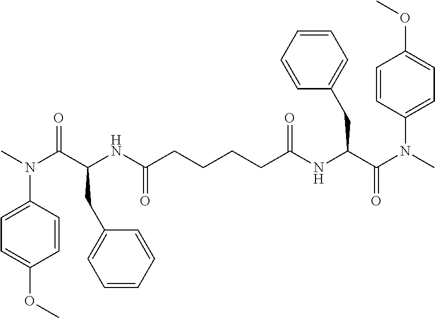 [SMILES: COc1ccc(N(C)C(=O)[C@H](Cc2ccccc2)NC(=O)CCCCC(=O)N[C@@H](Cc2ccccc2)C(=O)N(C)c2ccc(OC)cc2)cc1]

A solution of an HCl salt of Intermediate 4 (42 mg, 0.13 mmol) in DCM (0.5 mL) was added to a stirred solution of adipoyl dichloride (11 mg, 0.059 mmol) in DCM (0.5 mL) at rt. Then TEA (0.05 mL, 0.36 mmol) was added and the reaction mixture was stirred at rt for 4 h. The reaction mixture was concentrated and the residue was dissolved into MeOH, filtered and purified by preparative HPLC to yield the title compound (33.5 mg). LC-MS retention time=1.87 min; m/z=679.7 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 24

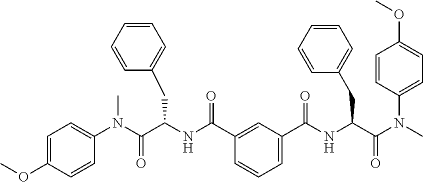 [SMILES: COc1ccc(N(C)C(=O)[C@H](Cc2ccccc2)NC(=O)c2cccc(C(=O)N[C@@H](Cc3ccccc3)C(=O)N(C)c3ccc(OC)cc3)c2)cc1]

A solution of an HCl salt of Intermediate 4 (42 mg, 0.13 mmol) in DCM (0.5 mL) was added to a stirred solution of isophthaloyl dichloride (12 mg, 0.059 mmol) in DCM (0.5 mL) at rt. Then TEA (0.05 mL, 0.36 mmol) was added and the reaction mixture was stirred at rt for 4 h. The reaction mixture was concentrated and the residue was dissolved into MeOH, filtered and purified by preparative HPLC to yield the title compound (36.1 mg). LC-MS retention time=2.06 min; m/z=699.6 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, DMSO-d₆) δ ppm 8.80 (d, J=7.7 Hz, 2H), 8.27 (s, 1H), 7.92 (d, J=7.7 Hz, 2H), 7.52 (t, J=7.7 Hz, 1H), 7.28 (d, J=6.2 Hz, 4H), 7.20-7.11 (m, 6H), 7.04 (d, J=8.8 Hz, 4H), 6.90 (d, J=7.3 Hz, 4H), 4.70-4.63 (m, 2H), 3.82 (s, 6H), 3.16 (s, 6H), 2.98-2.87 (m, 4H).

### Example 25

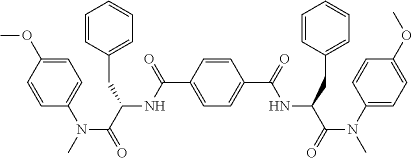 [SMILES: COc1ccc(N(C)C(=O)[C@H](Cc2ccccc2)NC(=O)c2ccc(C(=O)N[C@@H](Cc3ccccc3)C(=O)N(C)c3ccc(OC)cc3)cc2)cc1]

A solution of an HCl salt of Intermediate 4 (42 mg, 0.13 mmol) in DCM (0.5 mL) was added to a stirred solution of terephthaloyl dichloride (12 mg, 0.059 mmol) in DCM (0.5 mL) at rt. Then TEA (0.05 mL, 0.36 mmol) was added and the reaction mixture was stirred at rt for 3 d. The reaction mixture was concentrated and the residue was dissolved into MeOH, filtered and purified by preparative HPLC to yield the title compound (22.9 mg). LC-MS retention time=2.01 min; m/z=699.7 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 26

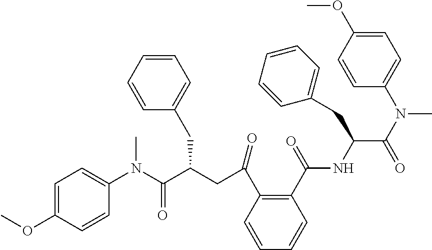

A solution of an HCl salt of Intermediate 4 (42 mg, 0.13 mmol) in DCM (0.5 mL) was added to a stirred solution of phthaloyl dichloride (12 mg, 0.059 mmol) in DCM (0.5 mL) at rt. Then TEA (0.05 mL, 0.36 mmol) was added and the reaction mixture was stirred at rt for 4 h. The reaction mixture was concentrated and the residue was dissolved into MeOH, filtered and purified by preparative HPLC to yield the title compound (16.4 mg). LC-MS retention time=2.00 min; m/z=699.6 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 27

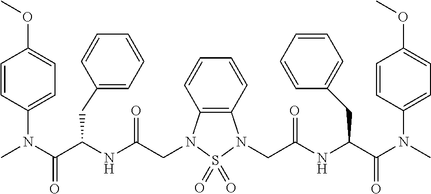 [SMILES: COc1ccc(N(C)C(=O)[C@H](Cc2ccccc2)NC(=O)CN2c3ccccc3N(CC(=O)N[C@@H](Cc3ccccc3)C(=O)N(C)c3ccc(OC)cc3)S2(=O)=O)cc1]

To a solution of an HCl salt of Intermediate 4 (30.8 mg, 0.096 mmol), Intermediate 51 (25 mg, 0.087 mmol) and DIPEA (0.061 mL, 0.35 mmol) in DMF (1 mL) was added HATU (33.2 mg, 0.087 mmol). The reaction mixture was stirred at rt overnight and then purified by preparative HPLC (Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-100% B over 20 minutes, then a 5-minute hold at 100% B; Flow: 20 mL/min. Fractions containing the title compound were combined and dried via centrifugal evaporation) to afford the title compound (17.7 mg) as a white solid. LC-MS retention time=1.34 min; m/z=819.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 28

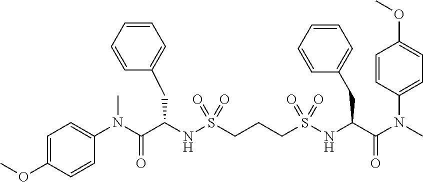 [SMILES: COc1ccc(N(C)C(=O)[C@H](Cc2ccccc2)NS(=O)(=O)CCCS(=O)(=O)N[C@@H](Cc2ccccc2)C(=O)N(C)c2ccc(OC)cc2)cc1]

A solution of propane-1,3-disulfonyl dichloride (18 mg, 0.075 mmol) was dissolved into DCM (0.5 mL) and then treated with a solution of an HCl salt of Intermediate 4 (52.7 mg, 0.164 mmol) in DCM (0.5 mL) followed by TEA (0.06 mL, 0.45 mmol) and the reaction mixture was stirred at rt for 3 d. The reaction mixture was concentrated and the residue was dissolved into MeOH, filtered and purified by preparative HPLC to yield the title compound (2.8 mg). LC-MS retention time=1.98 min; m/z=737.6 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 29

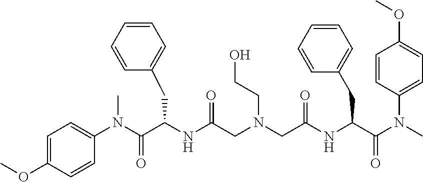 [SMILES: COc1ccc(N(C)C(=O)[C@H](Cc2ccccc2)NC(=O)CN(CCO)CC(=O)N[C@@H](Cc2ccccc2)C(=O)N(C)c2ccc(OC)cc2)cc1]

HATU (64 mg, 0.17 mmol) was added to a stirred solution of 2,2′-((2-hydroxyethyl)azanediyl)diacetic acid (13.6 mg, 0.077 mol) and an HCl salt of Intermediate 4 (54 mg, 0.17 mmol) in DMF (1 mL) and DIPEA (0.054 mL, 0.31 mmol) and the reaction mixture was stirred at rt overnight. The reaction was partially concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (11.3 mg). LC-MS retention time=1.86 min; m/z=710.6 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 30

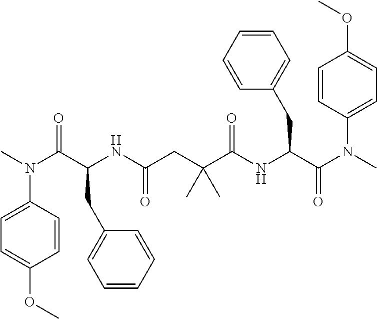 [SMILES: COc1ccc(N(C)C(=O)[C@H](Cc2ccccc2)NC(=O)CC(C)(C)C(=O)N[C@@H](Cc2ccccc2)C(=O)N(C)c2ccc(OC)cc2)cc1]

HATU (64 mg, 0.17 mmol) was added to a stirred solution of 2,2-dimethylsuccinic acid (11 mg, 0.077 mmol) and an HCl salt of Intermediate 4 (54 mg, 0.17 mmol) in DMF (1 mL) and DIPEA (0.054 mL, 0.31 mmol) and the reaction mixture was stirred at rt overnight. The reaction was partially concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (35 mg). LC-MS retention time=2.05 min; m/z=679.6 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 31

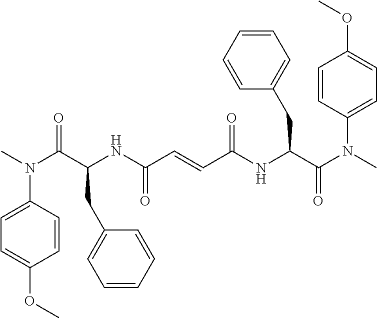 [SMILES: COc1ccc(N(C)C(=O)[C@H](Cc2ccccc2)NC(=O)/C=C/C(=O)N[C@@H](Cc2ccccc2)C(=O)N(C)c2ccc(OC)cc2)cc1]

HATU (64 mg, 0.17 mmol) was added to a stirred solution of fumaric acid (8.9 mg, 0.077 mmol) and an HCl salt of Intermediate 4 (54 mg, 0.17 mmol) in DMF (1 mL) and DIPEA (0.053 mL, 0.31 mmol) and the reaction mixture was stirred at rt overnight. The reaction was partially concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (35 mg). LC-MS retention time=1.88 min; m/z=649.6 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 32

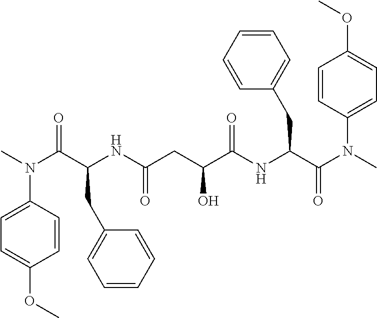 [SMILES: COc1ccc(N(C)C(=O)[C@H](Cc2ccccc2)NC(=O)C[C@H](O)C(=O)N[C@@H](Cc2ccccc2)C(=O)N(C)c2ccc(OC)cc2)cc1]

HATU (64 mg, 0.17 mmol) was added to a stirred solution of (S)-2-hydroxysuccinic acid (10.3 mg, 0.077 mmol) and an HCl salt of Intermediate 4 (54 mg, 0.17 mmol) in DMF (1 mL) and DIPEA (0.053 mL, 0.31 mmol) and the reaction mixture was stirred at rt overnight. The reaction was partially concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (35 mg). LC-MS retention time=1.85 min; m/z=667.5 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 33

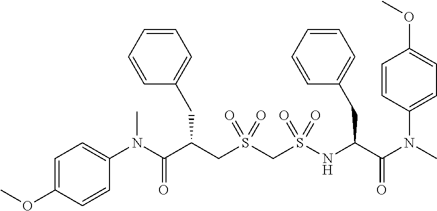 [SMILES: COc1ccc(N(C)C(=O)[C@H](Cc2ccccc2)CS(=O)(=O)CS(=O)(=O)N[C@@H](Cc2ccccc2)C(=O)N(C)c2ccc(OC)cc2)cc1]

A solution of methanedisulfonyl dichloride (16 mg, 0.075 mmol) was dissolved into DCM (0.5 mL) and then treated with a solution of an HCl salt of Intermediate 4 (52.7 mg, 0.164 mmol) in DCM (0.5 mL) followed by TEA (0.06 mL, 0.5 mmol) and the reaction mixture was stirred at rt for 3 d. The reaction mixture was concentrated and the residue was dissolved into MeOH, filtered and purified by preparative HPLC to yield the title compound (12 mg). LC-MS retention time=2.04 min; m/z=709.5 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 34

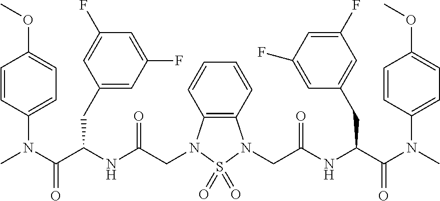 [SMILES: COc1ccc(N(C)C(=O)[C@H](Cc2cc(F)cc(F)c2)NC(=O)CN2c3ccccc3N(CC(=O)N[C@@H](Cc3cc(F)cc(F)c3)C(=O)N(C)c3ccc(OC)cc3)S2(=O)=O)cc1]

To a solution of an HCl salt of Intermediate 13 (49.7 mg, 0.139 mmol), Intermediate 51 (19 mg, 0.066 mmol) and DIPEA (93 μ1, 0.53 mmol) in DMF (1 mL) was added HATU (55.5 mg, 0.146 mmol). The reaction mixture was stirred at rt overnight and then purified by preparative HPLC (Column: Waters Sunfire C18 OBD, 30×100 mm, 5-μm particles; Mobile Phase A: 90:10 methanol: water with 0.1% TFA; Mobile Phase B: 10:90 methanol: water with 0.1% TFA; Gradient: 30-100% B over 15 minutes, then a 5-minute hold at 100% B; Flow: 30 mL/min. Fractions containing the title compound were combined and dried via centrifugal evaporation) to afford the title compound (29 mg) as a white solid. LC-MS retention time=1.39 min; m/z=891.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm). ¹H NMR (400 MHZ, methanol-d₄) δ ppm 7.13 (br. s., 3H), 7.03-6.96 (m, 7H), 6.81-6.72 (m, 2H), 6.69 (dd, J=5.8, 3.3 Hz, 2H), 6.43 (d, J=6.3 Hz, 4H), 4.82-4.66 (m, 2H), 4.40 (s, 4H), 3.84 (s, 6H), 3.22 (s, 6H), 2.96 (dd, J=13.6, 5.8 Hz, 2H), 2.77 (dd, J=13.6, 8.5 Hz, 2H).

### Example 35

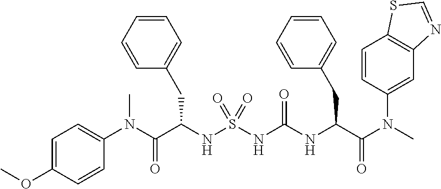 [SMILES: COc1ccc(N(C)C(=O)[C@H](Cc2ccccc2)NS(=O)(=O)NC(=O)N[C@@H](Cc2ccccc2)C(=O)N(C)c2ccc3scnc3c2)cc1]

At 0° C., a solution of an HCl salt of Intermediate 18 (50 mg, 0.13 mmol) and TEA (0.036 mL, 0.26 mmol) in DCM (0.5 mL) was added to a solution of sulfurisocyanatidic chloride (0.012 mL, 0.14 mmol) in DCM (0.5 mL) and the reaction mixture was stirred at 0° C. for 30 min. Then a solution of an HCl salt of Intermediate 4 (50 mg, 0.15 mmol) in DCM (0.5 mL) and TEA (0.054 mL, 0.39 mmol) was added to the reaction mixture and it was stirred at rt for 2 h. The reaction mixture was concentrated, the residue was dissolved in MeOH, and then purified via preparative HPLC (Column: XBridge C18, 19×200 mm, 5-μm particles; Mobile Phase A: 5:95 acetonitrile: water with 0.1% TFA; Mobile Phase B: 95:5 acetonitrile: water with 0.1% TFA; Gradient: 40-80% B over 20 minutes, then a 5-minute hold at 100% B; Flow: 20 mL/min. Fractions containing the title compound were combined and dried via centrifugal evaporation. The material was further purified via preparative HPLC with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation.) to yield the title compound (4.0 mg). LC-MS retention time=1.81 min; m/z=701.0 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=1.0 mL/min. Start % B=0. Final % B=100. Gradient Time=3 minutes, then a 0.5-minute hold at 100% B. Wavelength=220).

### Example 36

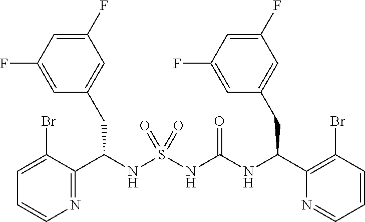 [SMILES: O=C(N[C@@H](Cc1cc(F)cc(F)c1)c1ncccc1Br)NS(=O)(=O)N[C@@H](Cc1cc(F)cc(F)c1)c1ncccc1Br]

A solution of 4 M HCl (1.0 mL, 4.0 mmol) in 1,4-dioxane was added to a stirred solution of Intermediate 15 (448 mg, 1.07 mmol) in MeOH (1.7 mL) and the reaction mixture was stirred at rt overnight. The crude reaction mixture was concentrated to dryness, and dissolved into DCM (˜5 mL) and TEA (0.60 mL, 4.3 mmol). The reaction solution was cooled to 0° C. and then treated dropwise with a solution of sulfurisocyanatidic chloride (76 mg, 0.54 mmol) in DCM (1 mL). The reaction mixture was stirred at 0° C. for 30 min and then allowed to warm to rt and stirred for 3 h. The reaction was concentrated and purified using a Biotage Horizon (24 g SiO₂, 30-100% EtOAc/hexanes) to yield the title compound (185 mg) as a yellow solid. About 20% of this material was further purified by preparative HPLC to yield the title compound (16.9 mg, 97% purity). LC-MS retention time=2.11 min; m/z=730.0 [M−H]⁻. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 37

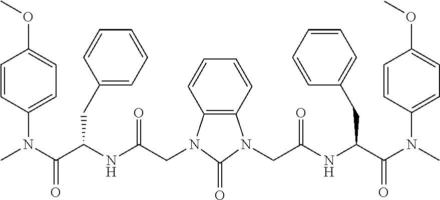 [SMILES: COc1ccc(N(C)C(=O)[C@H](Cc2ccccc2)NC(=O)Cn2c(=O)n(CC(=O)N[C@@H](Cc3ccccc3)C(=O)N(C)c3ccc(OC)cc3)c3ccccc32)cc1]

To a solution of an HCl salt of Intermediate 4 (51.2 mg, 0.159 mmol), Intermediate 52 (19 mg, 0.076 mmol) and DIPEA (0.11 m, 0.61 mmol) in DMF (0.9 mL) was added HATU (63.5 mg, 0.167 mmol). The reaction mixture was stirred at rt for 2 h and then purified by preparative HPLC to afford the title compound (45 mg) as a white solid. LC-MS retention time=1.32 min; m/z=783.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 38

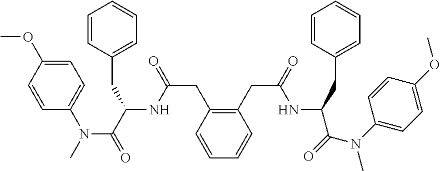 [SMILES: COc1ccc(N(C)C(=O)[C@H](Cc2ccccc2)NC(=O)Cc2ccccc2CC(=O)N[C@@H](Cc2ccccc2)C(=O)N(C)c2ccc(OC)cc2)cc1]

HATU (63.3 mg, 0.167 mmol) was added to a stirred solution of 2,2′-(1,2-phenylene)diacetic acid (15.4 mg, 0.079 mmol) and an HCl salt of Intermediate 4 (57.0 mg, 0.174 mmol) in DMF (1 mL) and DIPEA (0.08 mL, 0.5 mmol) and the reaction mixture was stirred at rt for 2d. The reaction was partially concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (14 mg). LC-MS retention time=2.37 min; m/z=727.3 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 39

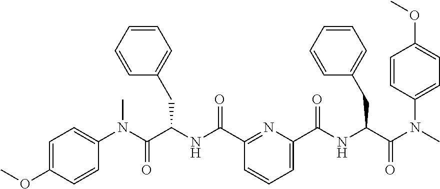 [SMILES: COc1ccc(N(C)C(=O)[C@H](Cc2ccccc2)NC(=O)c2cccc(C(=O)N[C@@H](Cc3ccccc3)C(=O)N(C)c3ccc(OC)cc3)n2)cc1]

HATU (63.3 mg, 0.167 mmol) was added to a stirred solution of pyridine-2,6-dicarboxylic acid (13.3 mg, 0.079 mmol) and an HCl salt of Intermediate 4 (57.0 mg, 0.174 mmol) in DMF (1 mL) and DIPEA (0.08 mL, 0.5 mmol) and the reaction mixture was stirred at rt for 2d. The reaction was partially concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (40.3 mg). LC-MS retention time=2.29 min; m/z=700.3 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 40

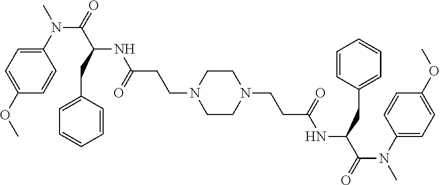 [SMILES: COc1ccc(N(C)C(=O)[C@H](Cc2ccccc2)NC(=O)CCN2CCN(CCC(=O)N[C@@H](Cc3ccccc3)C(=O)N(C)c3ccc(OC)cc3)CC2)cc1]

HATU (63.2 mg, 0.166 mmol) was added to a stirred mixture of 3,3′-(piperazine-1,4-diyl)dipropanoic acid (18.2 mg, 0.079 mmol) and Intermediate 4 (56.9 mg, 0.174 mmol) in DMF (1 mL) and DIPEA (0.08 mL, 0.5 mmol) and the reaction mixture was stirred at rt for 2 d. The reaction was partially concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (4.3 mg). LC-MS retention time=1.76 min; m/z=763.4 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 41

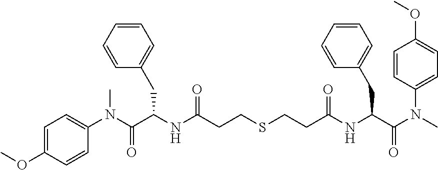 [SMILES: COc1ccc(N(C)C(=O)[C@H](Cc2ccccc2)NC(=O)CCSCCC(=O)N[C@@H](Cc2ccccc2)C(=O)N(C)c2ccc(OC)cc2)cc1]

HATU (63.3 mg, 0.167 mmol) was added to a stirred solution of 3,3′-thiodipropanoic acid (14.1 mg, 0.079 mmol) and an HCl salt of Intermediate 4 (57.0 mg, 0.174 mmol) in DMF (1 mL) and DIPEA (0.08 mL, 0.5 mmol) and the reaction mixture was stirred at rt for 2 d. The reaction was partially concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (35 mg). LC-MS retention time=2.01 min; m/z=711.3 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, DMSO-d₆) δ ppm 8.26 (d, J=7.7 Hz, 2H), 7.21-7.13 (m, 6H), 7.06 (br. s., 4H), 6.95 (d, J=8.4 Hz, 4H), 6.87 (d, J=6.6 Hz, 4H), 4.48-4.40 (m, 2H), 3.78 (s, 6H), 3.08 (s, 6H), 2.84 (dd, J=13.2, 5.1 Hz, 2H), 2.63 (dd, J=13.2, 9.2 Hz, 2H), 2.56-2.45 (m, 4H), 2.28 (t, J=7.3 Hz, 4H).

### Example 42

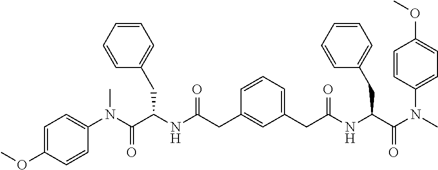 [SMILES: COc1ccc(N(C)C(=O)[C@H](Cc2ccccc2)NC(=O)Cc2cccc(CC(=O)N[C@@H](Cc3ccccc3)C(=O)N(C)c3ccc(OC)cc3)c2)cc1]

HATU (44.3 mg, 0.116 mmol) was added to a stirred mixture of an HCl salt of Intermediate 4 (45 mg, 0.12 mmol) and 2,2′-(1,3-phenylene)diacetic acid (11 mg, 0.055 mmol) in DMF (1 mL) and DIPEA (0.058 mL, 0.333 mmol) and the reaction mixture was stirred at rt for 3 h. The reaction was partially concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (10.5 mg). LC-MS retention time=2.05 min; m/z=727.3 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 43

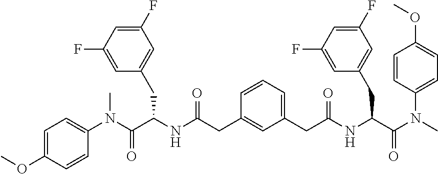 [SMILES: COc1ccc(N(C)C(=O)[C@H](Cc2cc(F)cc(F)c2)NC(=O)Cc2cccc(CC(=O)N[C@@H](Cc3cc(F)cc(F)c3)C(=O)N(C)c3ccc(OC)cc3)c2)cc1]

HATU (44.3 mg, 0.116 mmol) was added to a stirred mixture of an HCl salt of Intermediate 13 (43.5 mg, 0.122 mmol) and 2,2′-(1,3-phenylene)diacetic acid (11 mg, 0.055 mmol) in DMF (1 mL) and DIPEA (0.058 mL, 0.33 mmol) and the reaction mixture was stirred at rt for 3 h. The reaction was partially concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (10.5 mg). LC-MS retention time=2.16 min; m/z=799.3 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 44

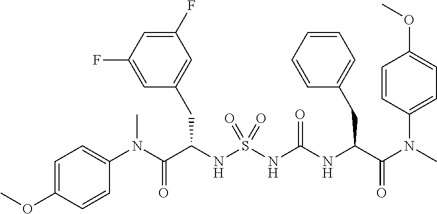 [SMILES: COc1ccc(N(C)C(=O)[C@H](Cc2ccccc2)NC(=O)NS(=O)(=O)N[C@@H](Cc2cc(F)cc(F)c2)C(=O)N(C)c2ccc(OC)cc2)cc1]

A solution of an HCl salt of Intermediate 4 (25 mg, 0.068 mmol) in DCM (0.2 mL) was added dropwise to a solution of sulfurisocyanatidic chloride (13.4 mg, 0.095 mmol) in DCM (0.5 mL) at 0° C. and the reaction was stirred for 0.5 h at 0° C. Then a solution of an HCl salt of Intermediate 13 (24.2 mg, 0.068 mmol) in DCM (0.3 mL) and TEA (0.7 mL, 0.4 mmol) was added to the reaction mixture and it was allowed to stirred at rt for 30 min. The crude reaction mixture was concentrated and the residue was dissolved into MeOH, filtered and purified by preparative HPLC to yield the title compound (37.4 mg). LC-MS retention time=2.02 min; m/z=710.2 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 45

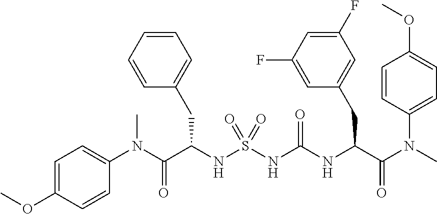 [SMILES: COc1ccc(N(C)C(=O)[C@H](Cc2cc(F)cc(F)c2)NC(=O)NS(=O)(=O)N[C@@H](Cc2ccccc2)C(=O)N(C)c2ccc(OC)cc2)cc1]

A solution of an HCl salt of Intermediate 13 (24 mg, 0.068 mmol) in DCM (0.2 mL) and TEA (one drop) was added dropwise to a solution of sulfurisocyanatidic chloride (13.4 mg, 0.095 mmol) in DCM (0.5 mL) at 0° C. and the reaction was stirred for 0.5 h at 0° C. Then a solution of an HCl salt of Intermediate 4 (25 mg, 0.068 mmol) in DCM (0.3 mL) and TEA (0.07 mL, 0.4 mmol). The crude reaction mixture was concentrated and the residue was dissolved into MeOH, filtered and purified by preparative HPLC to yield the title compound (9.7 mg). LC-MS retention time=1.99 min; m/z=710.1 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, DMSO-d₆) δ ppm 7.28-7.12 (m, 6H), 7.09-6.88 (m, 8H), 6.81 (d, J=4.0 Hz, 2H), 6.55 (d, J=7.0 Hz, 3H), 4.49-4.43 (m, 1H), 4.11 (d, J=7.0 Hz, 1H), 3.79 (s, 3H), 3.76 (s, 3H), 3.11 (s, 3H), 2.98 (s, 3H), 2.83 (dd, J=13.6, 5.1 Hz, 1H), 2.79-2.73 (m, 1H), 2.67-2.56 (m, 2H).

### Example 46

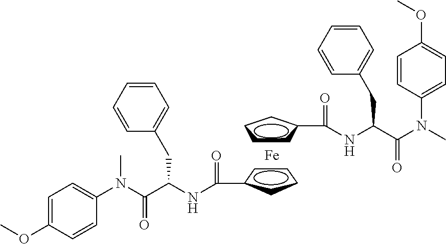

HATU (63.2 mg, 0.166 mmol) was added to a stirred mixture of 1,1′-ferrocenedicarboxylic acid (21.7 mg, 0.079 mmol) and Intermediate 4 (56.9 mg, 0.174 mmol) in DMF (1 mL) and DIPEA (0.08 mL, 0.5 mmol) and the reaction mixture was stirred at rt for 2 d. The reaction was partially concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (34.3 mg). LC-MS retention time=2.48 min; m/z=807.2 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 47

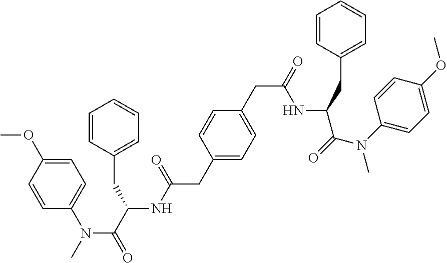 [SMILES: COc1ccc(N(C)C(=O)[C@H](Cc2ccccc2)NC(=O)Cc2ccc(CC(=O)N[C@@H](Cc3ccccc3)C(=O)N(C)c3ccc(OC)cc3)cc2)cc1]

To a solution of an HCl salt of Intermediate 4 (40 mg, 0.13 mmol), 2,2′-(1,4-phenylene)diacetic acid (11.5 mg, 0.059 mmol) and DIPEA (0.083 mL, 0.475 mmol) in DMF (0.9 mL) was added HATU (49.7 mg, 0.131 mmol). The reaction mixture was stirred at rt for 2 h and then purified by preparative HPLC to afford the title compound as a white solid (21 mg). LC-MS retention time=1.27 min; m/z=727.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 48

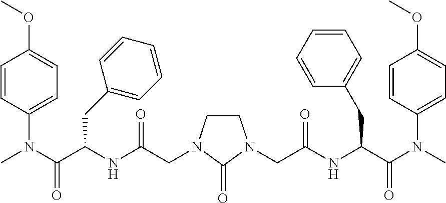 [SMILES: COc1ccc(N(C)C(=O)[C@H](Cc2ccccc2)NC(=O)CN2CCN(CC(=O)N[C@@H](Cc3ccccc3)C(=O)N(C)c3ccc(OC)cc3)C2=O)cc1]

To a solution of an HCl salt of Intermediate 4 (37.3 mg, 0.116 mmol), Intermediate 53 (14 mg, 0.055 mmol) and DIPEA (0.077 mL, 0.44 mmol) in DMF (0.8 mL) was added HATU (46.3 mg, 0.122 mmol). The reaction mixture was stirred at rt overnight and then purified by preparative HPLC to afford the title compound as a white solid (6.1 mg). LC-MS retention time=1.20 min; m/z=735.3 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 49

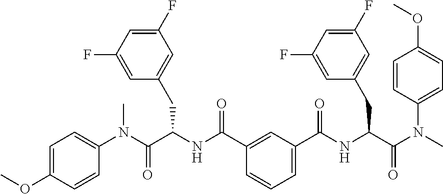 [SMILES: COc1ccc(N(C)C(=O)[C@H](Cc2cc(F)cc(F)c2)NC(=O)c2cccc(C(=O)N[C@@H](Cc3cc(F)cc(F)c3)C(=O)N(C)c3ccc(OC)cc3)c2)cc1]

HATU (48 mg, 0.13 mmol) was added to a stirred solution of an HCl salt of Intermediate 13 (47 mg, 0.13 mmol) and isophthalic acid (10 mg, 0.060 mmol) in DMF (1 mL) and DIPEA (0.06 mL, 0.4 mmol) and the reaction mixture was stirred at rt for 3 h. The reaction mixture was concentrated and the residue was dissolved into MeOH, filtered and purified by preparative HPLC to yield the title compound (33.4 mg). LC-MS retention time=2.29 min; m/z=771.1 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, DMSO-d₆) δ ppm 8.87 (d, J=7.7 Hz, 2H), 8.30 (s, 1H), 7.94 (d, J=8.4 Hz, 2H), 7.56 (t, J=7.7 Hz, 1H), 7.39 (d, J=8.4 Hz, 4H), 7.08 (d, J=8.8 Hz, 4H), 7.00 (t, J=9.2 Hz, 2H), 6.53 (d, J=6.6 Hz, 4H), 4.72-4.63 (m, 2H), 3.82 (s, 6H), 3.18 (s, 6H), 3.03-2.92 (m, 4H).

### Example 50

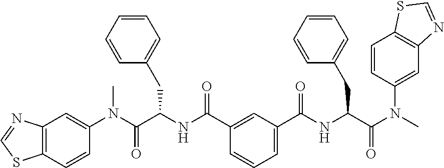 [SMILES: CN(C(=O)[C@H](Cc1ccccc1)NC(=O)c1cccc(C(=O)N[C@@H](Cc2ccccc2)C(=O)N(C)c2ccc3scnc3c2)c1)c1ccc2scnc2c1]

HATU (48 mg, 0.13 mmol) was added to a stirred solution of an HCl salt of Intermediate 18 (46 mg, 0.13 mmol) and isophthalic acid (10 mg, 0.060 mmol) in DMF (1 mL) and DIPEA (0.06 mL, 0.4 mmol) and the reaction mixture was stirred at rt for 3 h. The reaction mixture was concentrated and the residue was dissolved into MeOH, filtered and purified by preparative HPLC to yield the title compound (15.1 mg). LC-MS retention time=1.97 min; m/z=753.1 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, DMSO-d₆) δ ppm 9.50 (s, 2H), 8.90 (d, J=7.3 Hz, 2H), 8.34-8.24 (m, 3H), 8.07 (br. s., 2H), 7.94 (d, J=7.7 Hz, 2H), 7.57-7.41 (m, 3H), 7.11 (br. s., 6H), 6.85 (br. s., 4H), 4.68 (d, J=4.4 Hz, 2H), 3.28 (s, 6H), 3.10-2.92 (m, 4H).

### Example 51

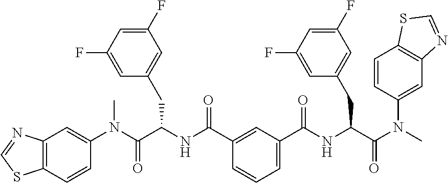 [SMILES: CN(C(=O)[C@H](Cc1cc(F)cc(F)c1)NC(=O)c1cccc(C(=O)N[C@@H](Cc2cc(F)cc(F)c2)C(=O)N(C)c2ccc3scnc3c2)c1)c1ccc2scnc2c1]

HATU (48 mg, 0.13 mmol) was added to a stirred solution of an HCl salt of Intermediate 20 (51 mg, 0.13 mmol) and isophthalic acid (10 mg, 0.060 mmol) in DMF (1 mL) and DIPEA (0.06 mL, 0.4 mmol) and the reaction mixture was stirred at rt for 3 h. The reaction mixture was concentrated and the residue was dissolved into MeOH, filtered and purified by preparative HPLC to yield the title compound (29.3 mg). LC-MS retention time=2.08 min; m/z=825.0 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, DMSO-d₆) δ ppm 9.50 (s, 2H), 8.90 (d, J=7.3 Hz, 2H), 8.34-8.24 (m, 3H), 8.07 (br. s., 2H), 7.94 (d, J=7.7 Hz, 2H), 7.57-7.41 (m, 3H), 7.11 (br. s., 6H), 6.85 (br. s., 4H), 4.68 (d, J=4.4 Hz, 2H), 3.28 (s, 6H), 3.10-2.92 (m, 4H).

### Example 52

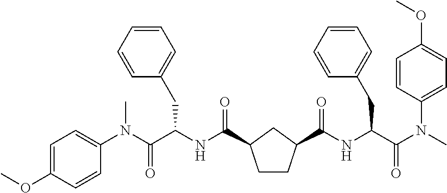 [SMILES: COc1ccc(N(C)C(=O)[C@H](Cc2ccccc2)NC(=O)[C@@H]2CC[C@H](C(=O)N[C@@H](Cc3ccccc3)C(=O)N(C)c3ccc(OC)cc3)C2)cc1]

HATU (61 mg, 0.16 mmol) was added to a stirred solution of cis cyclopentane-1,3-dicarboxylic acid (12 mg, 0.076 mmol) and an HCl salt of Intermediate 4 (54 mg, 0.17 mmol) in DMF (1 mL) and DIPEA (0.08 mL, 0.5 mmol) and the reaction mixture was stirred at rt for 3 h. The reaction was partially concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (35.7 mg). LC-MS retention time=2.07 min; m/z=691.2 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 53

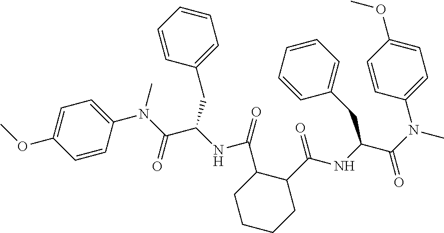

HATU (61 mg, 0.16 mmol) was added to a stirred solution of racemic trans cyclohexane-1,2-dicarboxylic acid (13 mg, 0.076 mmol) and an HCl salt of Intermediate 4 (54 mg, 0.17 mmol) in DMF (1 mL) and DIPEA (0.08 mL, 0.5 mmol) and the reaction mixture was stirred at rt for 3 h. The reaction was partially concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (17.6 mg). LC-MS retention time=2.21 min; m/z=705.2 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, DMSO-d₆) δ ppm 8.15 (dd, J=17.4, 7.9 Hz, 2H), 7.15 (br. s., 10H), 6.96 (t, J=8.6 Hz, 4H), 6.85 (d, J=6.6 Hz, 4H), 4.42 (d, J=3.7 Hz, 2H), 3.78 (s, 3H), 3.76 (s, 3H), 3.18 (s, 3H), 2.83 (d, J=11.0 Hz, 2H), 2.69-2.62 (m, 2H), 2.61-2.53 (m, 2H), 1.81-1.73 (m, 1H), 1.67-1.47 (m, 4H), 1.45-1.36 (m, 1H).

### Example 55

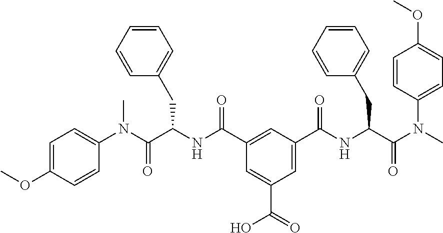 [SMILES: COc1ccc(N(C)C(=O)[C@H](Cc2ccccc2)NC(=O)c2cc(C(=O)O)cc(C(=O)N[C@@H](Cc3ccccc3)C(=O)N(C)c3ccc(OC)cc3)c2)cc1]

TEA (0.11 mL, 0.75 mmol) was added to a stirred solution of benzene-1,3,5-tricarbonyl trichloride (20 mg, 0.075 mmol) and Intermediate 4 (71 mg, 0.25 mmol) in DCM (1 mL) and the reaction mixture was stirred at rt overnight. The reaction mixture was concentrated and the residue was dissolved in MeOH, filtered and purified by preparative HPLC to yield the title compound (6.2 mg). LC-MS retention time=1.74 min; m/z=743.2 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, DMSO-d₆) δ ppm 9.01 (d, J=7.3 Hz, 2H), 8.51 (s, 2H), 8.41 (s, 1H), 7.26 (d, J=6.2 Hz, 4H), 7.20-7.11 (m, 6H), 7.03 (d, J=8.8 Hz, 4H), 6.91 (d, J=7.3 Hz, 4H), 4.74-4.68 (m, 2H), 3.91 (s, 3H), 3.16 (s, 6H), 2.97-2.92 (m, 4H).

### Example 56

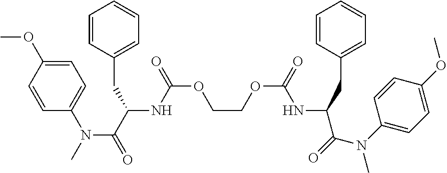 [SMILES: COc1ccc(N(C)C(=O)[C@H](Cc2ccccc2)NC(=O)OCCOC(=O)N[C@@H](Cc2ccccc2)C(=O)N(C)c2ccc(OC)cc2)cc1]

TEA (0.11 mL, 0.75 mmol) was added to a stirred solution of ethane-1,2-diyl dicarbonochloridate (21 mg, 0.11 mmol) and Intermediate 4 (71 mg, 0.25 mmol) in DCM (1 mL) and the reaction mixture was stirred at rt overnight. The reaction mixture was concentrated and the residue was dissolved in MeOH, filtered and purified by preparative HPLC to yield the title compound (29.3 mg). LC-MS retention time=2.13 min; m/z=683.1 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, DMSO-d₆) δ ppm 8.95 (d, J=7.7 Hz, 3H), 8.40 (s, 3H), 7.32-7.12 (m, 15H), 7.04 (d, J=8.8 Hz, 6H), 6.93 (d, J=7.0 Hz, 6H), 4.77-4.68 (m, 3H), 3.91 (s, 1H), 3.18 (s, 9H), 3.02-2.92 (m, 6H).

### Example 57

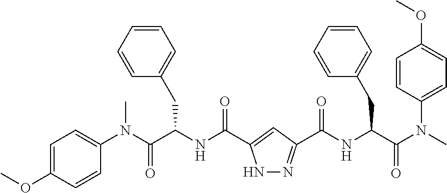 [SMILES: COc1ccc(N(C)C(=O)[C@H](Cc2ccccc2)NC(=O)c2cc(C(=O)N[C@@H](Cc3ccccc3)C(=O)N(C)c3ccc(OC)cc3)[nH]n2)cc1]

HATU (61 mg, 0.16 mmol) was added to a stirred solution of 1H-pyrazole-3,5-dicarboxylic acid (12 mg, 0.077 mmol) and an HCl salt of Intermediate 4 (54 mg, 0.17 mmol) in DMF (1 mL) and DIPEA (0.08 mL, 0.5 mmol) and the reaction mixture was stirred at rt for 3 h. The reaction mixture was concentrated, diluted with EtOAc (˜1.5 mL) and washed with water (1 mL) and then brine (1 mL). The organic component was concentrated and the residue was dissolved in MeOH, filtered and purified by preparative HPLC to yield the title compound (8.5 mg). LC-MS retention time=2.03 min; m/z=689.1 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, DMSO-d₆) δ ppm 8.90 (br. s., 1H), 7.96 (br. s., 1H), 7.37-7.10 (m, 11H), 7.02 (d, J=8.8 Hz, 4H), 6.97-6.84 (m, 4H), 4.66 (br. s., 2H), 3.82 (s, 6H), 3.15 (s, 6H), 2.98-2.82 (m, 4H).

### Example 58

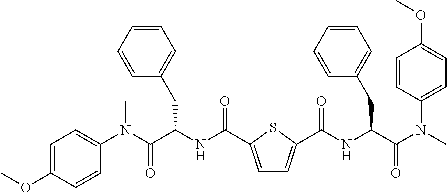 [SMILES: COc1ccc(N(C)C(=O)[C@H](Cc2ccccc2)NC(=O)c2ccc(C(=O)N[C@@H](Cc3ccccc3)C(=O)N(C)c3ccc(OC)cc3)s2)cc1]

HATU (61 mg, 0.16 mmol) was added to a stirred solution of thiophene-2,5-dicarboxylic acid (13 mg, 0.077 mmol) and an HCl salt of Intermediate 4 (54 mg, 0.17 mmol) in DMF (1 mL) and DIPEA (0.08 mL, 0.5 mmol) and the reaction mixture was stirred at rt for 3 h. The reaction mixture was concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (34.4 mg). LC-MS retention time=2.14 min; m/z=705.1 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, DMSO-d₆) δ ppm 8.90 (d, J=7.7 Hz, 2H), 7.83 (s, 2H), 7.26 (d, J=7.3 Hz, 4H), 7.21-7.11 (m, 6H), 7.03 (d, J=8.8 Hz, 4H), 6.88 (d, J=7.3 Hz, 4H), 4.63-4.55 (m, 2H), 3.81 (s, 6H), 3.14 (s, 6H), 2.97-2.85 (m, 4H).

### Example 59

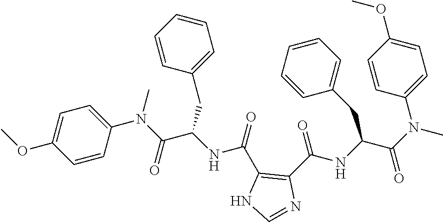

HATU (61 mg, 0.16 mmol) was added to a stirred solution of 1H-imidazole-4,5-dicarboxylic acid (12 mg, 0.077 mmol) and an HCl salt of Intermediate 4 (54 mg, 0.17 mmol) in DMF (1 mL) and DIPEA (0.08 mL, 0.5 mmol) and the reaction mixture was stirred at rt for 3 h. The reaction mixture was concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (8.5 mg). LC-MS retention time=2.26 min; m/z=689.2 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 61

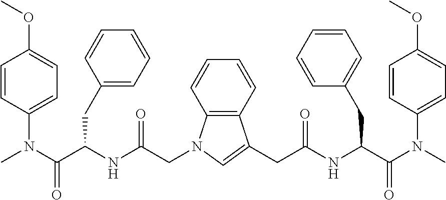 [SMILES: COc1ccc(N(C)C(=O)[C@H](Cc2ccccc2)NC(=O)Cc2cn(CC(=O)N[C@@H](Cc3ccccc3)C(=O)N(C)c3ccc(OC)cc3)c3ccccc23)cc1]

To a solution of an HCl salt of Intermediate 4 (57.8 mg, 0.180 mmol), Intermediate 54 (20 mg, 0.086 mmol) and DIPEA (0.12 mL, 0.69 mmol) in DMF (0.9 mL) was added HATU (71.7 mg, 0.189 mmol). The reaction mixture was stirred at rt for 2 h and then purified by preparative HPLC to afford the title compound as a white solid (59 mg) as a white solid. LC-MS retention time=1.36 min; m/z=766.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm). ¹H NMR (500 MHZ, DMSO-d₆) δ ppm 8.68 (d, J=7.7 Hz, 1H), 8.37 (d, J=8.1 Hz, 1H), 7.41-6.70 (m, 23H), 4.70 (q, J=16.1 Hz, 2H), 4.45 (dd, J=8.1, 5.1 Hz, 2H), 3.74 (d, J=5.9 Hz, 6H), 3.46-3.42 (m, 2H), 3.10 (d, J=4.0 Hz, 6H), 2.95-2.81 (m, 2H), 2.69 (dt, J=13.3, 8.9 Hz, 2H).

### Example 62

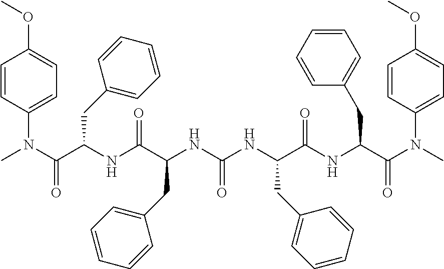 [SMILES: COc1ccc(N(C)C(=O)[C@H](Cc2ccccc2)NC(=O)[C@H](Cc2ccccc2)NC(=O)N[C@@H](Cc2ccccc2)C(=O)N[C@@H](Cc2ccccc2)C(=O)N(C)c2ccc(OC)cc2)cc1]

To a solution of an HCl salt of Intermediate 4 (28.4 mg, 0.088 mmol), Intermediate 55 (15 mg, 0.042 mmol) and DIPEA (0.059 mL, 0.34 mmol) in DMF (0.9 mL) was added HATU (35 mg, 0.093 mmol). The reaction mixture was stirred at rt for 2 h and then purified by preparative HPLC to afford the title compound as a white solid (26 mg). LC-MS retention time=1.41 min; m/z=889.3 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 63

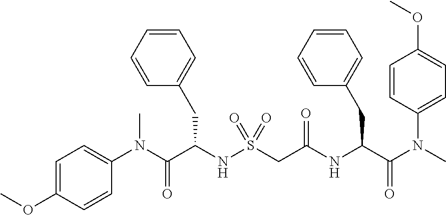 [SMILES: COc1ccc(N(C)C(=O)[C@H](Cc2ccccc2)NC(=O)CS(=O)(=O)N[C@@H](Cc2ccccc2)C(=O)N(C)c2ccc(OC)cc2)cc1]

A 1.1M solution of 2-(chlorosulfonyl)acetyl chloride (80 μL, 0.088 mmol) in DCM was added to a stirred solution of an HCl salt of Intermediate 4 (62 mg, 0.19 mmol) in DCM (1 mL) and TEA (61 μL, 0.44 mmol) and the reaction was stirred at rt. After 2 h of additional stirring,1.1 M solution of 2-(chlorosulfonyl)acetyl chloride (100 μL, 1.1 mmol) in DCM was added. After 30 min, additional 1.1 M solution of 2-(chlorosulfonyl)acetyl chloride (200 μL, 2.2 mmol) in DCM was added and the reaction was stirred at rt for 30 min. The reaction mixture was diluted with water (1 mL) and DCM (1 mL), stirred and separated. The organic component was concentrated, dissolved in MeOH, filtered and purified by preparative HPLC to yield the title compound (12.5 mg). LC-MS retention time=2.22 min; m/z=673.6 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, DMSO-d₆) δ ppm 8.73 (d, J=7.7 Hz, 1H), 7.66 (d, J=8.4 Hz, 1H), 7.21 (d, J=3.3 Hz, 6H), 7.09-6.83 (m, 12H), 4.49-4.39 (m, 1H), 4.17-4.10 (m, 1H), 3.77 (d, J=1.8 Hz, 6H), 3.70-3.62 (m, 2H), 3.12 (d, J=4.0 Hz, 6H), 2.89-2.84 (m, 2H), 2.72-2.61 (m, 2H).

### Example 64

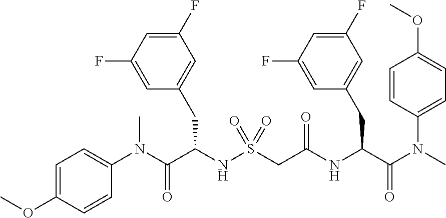 [SMILES: COc1ccc(N(C)C(=O)[C@H](Cc2cc(F)cc(F)c2)NC(=O)CS(=O)(=O)N[C@@H](Cc2cc(F)cc(F)c2)C(=O)N(C)c2ccc(OC)cc2)cc1]

A 1.1 M solution of 2-(chlorosulfonyl)acetyl chloride (80 μL, 0.088 mmol) in DCM was added to a stirred solution of an HCl salt of Intermediate 13 (69 mg, 0.19 mmol) in DCM (1 mL) and TEA (61 μL, 0.44 mmol) and the reaction was stirred at rt. After 2 h, additional 1.1 M solution of 2-(chlorosulfonyl)acetyl chloride (100 μL, 1.1 mmol) in DCM was added. After 30 min, additional 1.1 M solution of 2-(chlorosulfonyl)acetyl chloride (200 μL, 2.2 mmol) in DCM was added and the reaction was stirred at rt for 30 min. The reaction mixture was diluted with water (1 mL) and DCM (1 mL) stirred and separated. The organic component was concentrated, dissolved in MeOH, filtered and purified by preparative HPLC to yield the title compound (15.2 mg). LC-MS retention time=2.30 min; m/z=745.6 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, DMSO-d₆) δ ppm 8.72 (d, J=7.7 Hz, 1H), 7.73 (d, J=8.8 Hz, 1H), 7.20 (d, J=8.8 Hz, 4H), 7.05 (br. s., 2H), 6.98 (dd, J=8.8, 4.0 Hz, 4H), 6.56-6.45 (m, 4H), 4.47-4.41 (m, 1H), 4.17-4.10 (m, 1H), 3.80-3.72 (m, 8H), 3.13 (s, 3H), 3.11 (s, 3H), 2.89-2.79 (m, 2H), 2.72-2.64 (m, 2H).

### Example 65

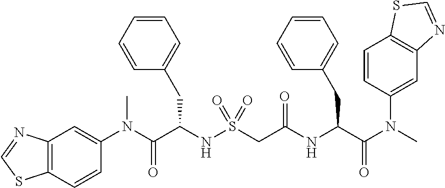 [SMILES: CN(C(=O)[C@H](Cc1ccccc1)NC(=O)CS(=O)(=O)N[C@@H](Cc1ccccc1)C(=O)N(C)c1ccc2scnc2c1)c1ccc2scnc2c1]

A 1.1 M solution of 2-(chlorosulfonyl)acetyl chloride (80 μL, 0.088 mmol) in DCM was added to a stirred solution of an HCl salt of Intermediate 18 (67 mg, 0.19 mmol) in DCM (1 mL) and TEA (61 μL, 0.44 mmol) and the reaction was stirred at rt. After 2 h, additional 1.1 M solution of 2-(chlorosulfonyl)acetyl chloride (100 μL, 1.1 mmol) in DCM was added. After 30 min, additional 1.1 M solution of 2-(chlorosulfonyl)acetyl chloride (200 μL, 2.2 mmol) in DCM was added and the reaction was stirred at rt for 30 min. The reaction mixture was diluted with water (1 mL) and DCM (1 mL), stirred and separated. The organic component was concentrated, dissolved into MeOH, filtered and purified by preparative HPLC to yield the title compound (5.6 mg). LC-MS retention time=1.91 min; m/z=727.5 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, DMSO-d₆) δ ppm 9.47 (d, J=12.8 Hz, 2H), 8.75 (d, J=7.7 Hz, 1H), 8.20-8.11 (m, 2H), 7.79 (d, J=8.4 Hz, 2H), 7.36-7.08 (m, 9H), 6.82 (t, J=7.0 Hz, 4H), 4.47-4.40 (m, 1H), 4.14 (d, J=7.0 Hz, 1H), 3.78-3.70 (m, 2H), 3.19 (d, J=4.8 Hz, 6H), 2.94-2.88 (m, 2H), 2.78-2.71 (m, 1H), 2.63 (dd, J=13.8, 7.5 Hz, 1H).

### Example 68

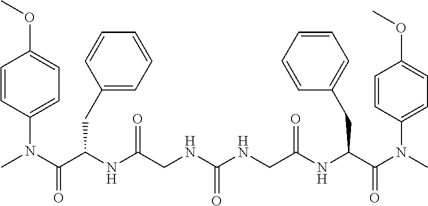 [SMILES: COc1ccc(N(C)C(=O)[C@H](Cc2ccccc2)NC(=O)CNC(=O)NCC(=O)N[C@@H](Cc2ccccc2)C(=O)N(C)c2ccc(OC)cc2)cc1]

To a solution of an HCl salt of Intermediate 4 (38.3 mg, 0.119 mmol), Intermediate 56 (10 mg, 0.057 mmol) and DIPEA (0.079 mL, 0.45 mmol) in DMF (0.8 mL) was added HATU (47.5 mg, 0.125 mmol). The reaction mixture was stirred at rt for 2 h and then purified by preparative HPLC to afford the title compound as a white solid (34 mg). LC-MS retention time=1.17 min; m/z=709.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 69

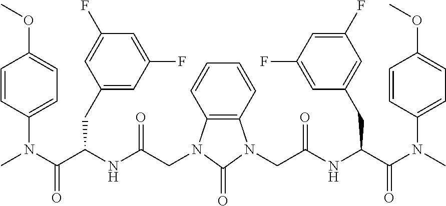 [SMILES: COc1ccc(N(C)C(=O)[C@H](Cc2cc(F)cc(F)c2)NC(=O)Cn2c(=O)n(CC(=O)N[C@@H](Cc3cc(F)cc(F)c3)C(=O)N(C)c3ccc(OC)cc3)c3ccccc32)cc1]

To a solution of an HCl salt of Intermediate 13 (44.9 mg, 0.126 mmol), Intermediate 52 (15 mg, 0.060 mmol) and DIPEA (0.084 mL, 0.48 mmol) in DMF (0.9 mL) was added HATU (50.1 mg, 0.132 mmol). The reaction mixture was stirred at rt for 2 h and then purified by preparative HPLC to afford the title compound as a white solid (33 mg). LC-MS retention time=1.35 min; m/z=855.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 70

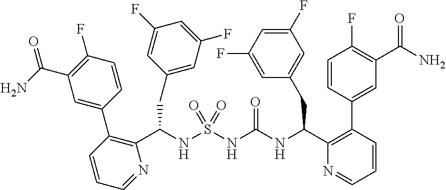 [SMILES: NC(=O)c1cc(-c2cccnc2[C@H](Cc2cc(F)cc(F)c2)NC(=O)NS(=O)(=O)N[C@@H](Cc2cc(F)cc(F)c2)c2ncccc2-c2ccc(F)c(C(N)=O)c2)ccc1F]

A solution of 4M HCl (0.128 mL, 0.513 mmol) in 1,4-dioxane was added to a stirred solution of Intermediate 25 (61 mg, 0.128 mmol) in methanol (1 mL) and the reaction mixture was stirred at rt overnight. The crude reaction mixture was concentrated to dryness, dissolved into DCM (0.5 mL) and TEA (0.05 mL, 0.4 mmol) and then treated with a solution of sulfurisocyanatidic chloride (13 mg, 0.090 mmol) in DCM (0.2 mL) and stirred at rt overnight. The crude reaction mixture was concentrated and the residue was dissolved into MeOH, filtered and purified by preparative HPLC to yield the title compound (8.4 mg).

LC-MS retention time=1.68 min; m/z=848.7 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, DMSO-d₆) δ ppm 8.49 (br. s., 1H), 8.41 (d, J=4.0 Hz, 1H), 7.95 (br. s., 1H), 7.81 (br. s., 1H), 7.63 (d, J=12.5 Hz, 2H), 7.53 (d, J=7.0 Hz, 2H), 7.37-7.27 (m, 4H), 7.18 (d, J=6.6 Hz, 2H), 7.00-6.94 (m, 2H), 6.87-6.76 (m, 2H), 6.31 (d, J=6.2 Hz, 2H), 6.19 (d, J=7.0 Hz, 2H), 5.03-4.95 (m, 1H), 4.64 (t, J=7.5 Hz, 1H), 2.79-2.72 (m, 4H).

### Example 71

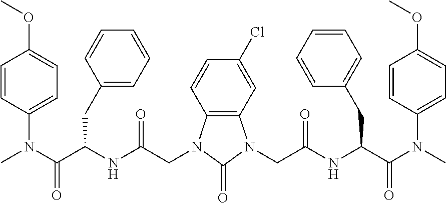 [SMILES: COc1ccc(N(C)C(=O)[C@H](Cc2ccccc2)NC(=O)Cn2c(=O)n(CC(=O)N[C@@H](Cc3ccccc3)C(=O)N(C)c3ccc(OC)cc3)c3cc(Cl)ccc32)cc1]

To a solution of an HCl salt of Intermediate 4 (35.5 mg, 0.111 mmol), Intermediate 57 (15 mg, 0.053 mmol) and DIPEA (0.074 mL, 0.42 mmol) in DMF (0.9 mL) was added HATU (44.1 mg, 0.116 mmol). The reaction mixture was stirred at rt for 2 h and then purified by preparative HPLC to afford the title compound as a white solid (33 mg). LC-MS retention time=1.38 min; m/z=817.3 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 72

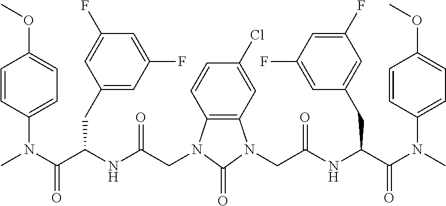 [SMILES: COc1ccc(N(C)C(=O)[C@H](Cc2cc(F)cc(F)c2)NC(=O)Cn2c(=O)n(CC(=O)N[C@@H](Cc3cc(F)cc(F)c3)C(=O)N(C)c3ccc(OC)cc3)c3cc(Cl)ccc32)cc1]

To a solution of an HCl salt of Intermediate 13 (39.5 mg, 0.111 mmol), Intermediate 57 (15 mg, 0.053 mmol) and DIPEA (0.074 mL, 0.42 mmol) in DMF (0.9 mL) was added HATU (44.1 mg, 0.116 mmol). The reaction mixture was stirred at rt for 2 h and then purified by preparative HPLC to afford the title compound as a white solid (9.4 mg). LC-MS retention time=1.41 min; m/z=889.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 73

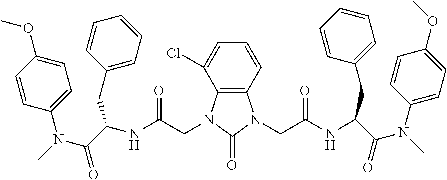 [SMILES: COc1ccc(N(C)C(=O)[C@H](Cc2ccccc2)NC(=O)Cn2c(=O)n(CC(=O)N[C@@H](Cc3ccccc3)C(=O)N(C)c3ccc(OC)cc3)c3c(Cl)cccc32)cc1]

To a solution of an HCl salt of Intermediate 4 (35.5 mg, 0.111 mmol), Intermediate 58 (15 mg, 0.053 mmol) and DIPEA (0.074 mL, 0.42 mmol) in DMF (0.9 mL) was added HATU (44.1 mg, 0.116 mmol). The reaction mixture was stirred at rt for 2 h and then purified by preparative HPLC to afford the title compound as a white solid (33 mg). LC-MS retention time=1.36 min; m/z=817.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 74

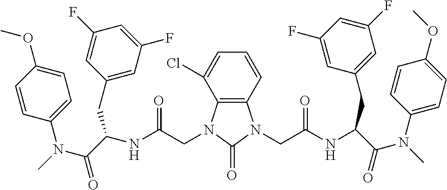 [SMILES: COc1ccc(N(C)C(=O)[C@H](Cc2cc(F)cc(F)c2)NC(=O)Cn2c(=O)n(CC(=O)N[C@@H](Cc3cc(F)cc(F)c3)C(=O)N(C)c3ccc(OC)cc3)c3c(Cl)cccc32)cc1]

To a solution of an HCl salt of Intermediate 13 (39.5 mg, 0.111 mmol), Intermediate 58 (15 mg, 0.053 mmol) and DIPEA (0.074 mL, 0.42 mmol) in DMF (0.9 mL) was added HATU (44.1 mg, 0.116 mmol). The reaction mixture was stirred at rt for 2 h and then purified by preparative HPLC to afford the title compound as a white solid (37 mg). LC-MS retention time=1.39 min; m/z=889.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 75

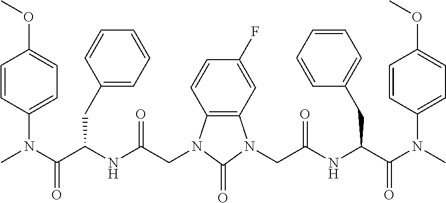 [SMILES: COc1ccc(N(C)C(=O)[C@H](Cc2ccccc2)NC(=O)Cn2c(=O)n(CC(=O)N[C@@H](Cc3ccccc3)C(=O)N(C)c3ccc(OC)cc3)c3cc(F)ccc32)cc1]

To a solution of an HCl salt of Intermediate 4 (35.5 mg, 0.111 mmol), Intermediate 59 (15 mg, 0.053 mmol) and DIPEA (0.07 mL, 0.4 mmol) in DMF (0.9 mL) was added HATU (44.1 mg, 0.116 mmol). The reaction mixture was stirred at rt for 2 h and then purified by preparative HPLC to afford the title compound as a white solid (30 mg). LC-MS retention time=1.32 min; m/z=801.3 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 76

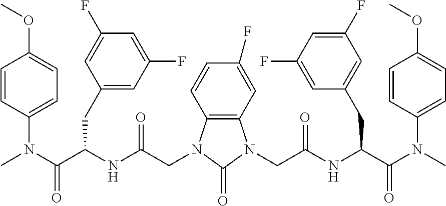 [SMILES: COc1ccc(N(C)C(=O)[C@H](Cc2cc(F)cc(F)c2)NC(=O)Cn2c(=O)n(CC(=O)N[C@@H](Cc3cc(F)cc(F)c3)C(=O)N(C)c3ccc(OC)cc3)c3cc(F)ccc32)cc1]

To a solution of an HCl salt of Intermediate 13 (36.3 mg, 0.102 mmol), Intermediate 59 (13 mg, 0.048 mmol) and DIPEA (0.07 mL, 0.4 mmol) in DMF (0.9 mL) was added HATU (40.5 mg, 0.107 mmol). The reaction mixture was stirred at rt for 2 h and then purified by preparative HPLC to afford the title compound as a white solid (31 mg). LC-MS retention time=1.36 min; m/z=873.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 77

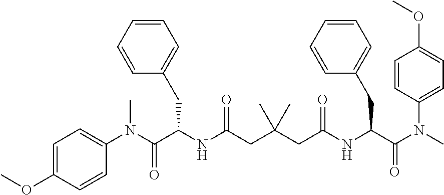 [SMILES: COc1ccc(N(C)C(=O)[C@H](Cc2ccccc2)NC(=O)CC(C)(C)CC(=O)N[C@@H](Cc2ccccc2)C(=O)N(C)c2ccc(OC)cc2)cc1]

HATU (68 mg, 0.18 mmol) was added to a stirred solution of 3,3-dimethylpentanedioic acid (14 mg, 0.085 mmol) and an HCl salt of Intermediate 4 (60 mg, 0.19 mmol) in DMF (1 mL) and DIPEA (0.09 mL, 0.5 mmol) and stirred at rt for 3 h. The reaction mixture was concentrated, diluted with EtOAc (˜1.5 mL) and washed with water (1 mL) and then brine (1 mL). The organic component was concentrated and the residue was dissolved in MeOH, filtered and purified by preparative HPLC to yield the title compound (9.7 mg). LC-MS retention time=2.25 min; m/z=693.4 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, methanol-d₄) δ ppm 7.35-7.15 (m, 10H), 7.03 (d, J=8.5 Hz, 4H), 6.92 (d, J=3.4 Hz, 4H), 4.70 (dd, J=9.8, 4.9 Hz, 2H), 3.87 (s, 6H), 3.28 (s, 6H), 3.01 (dd, J=13.6, 4.7 Hz, 2H), 2.78 (dd, J=13.7, 10.1 Hz, 2H), 2.23 (d, J=13.1 Hz, 2H), 1.91 (d, J=13.1 Hz, 2H), 0.98 (s, 6H).

### Example 78

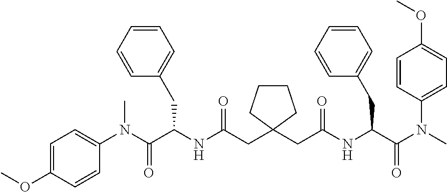 [SMILES: COc1ccc(N(C)C(=O)[C@H](Cc2ccccc2)NC(=O)CC2(CC(=O)N[C@@H](Cc3ccccc3)C(=O)N(C)c3ccc(OC)cc3)CCCC2)cc1]

HATU (68 mg, 0.18 mmol) was added to a stirred solution of 2,2′-(cyclopentane-1,1-diyl)diacetic acid (16 mg, 0.085 mmol) and an HCl salt of Intermediate 4 (60 mg, 0.19 mmol) in DMF (1 mL) and DIPEA (0.089 mL, 0.51 mmol) and the reaction mixture was stirred at rt for 3 h. The reaction mixture was concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (6.3 mg). LC-MS retention time=2.49 min; m/z=719.4 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 79

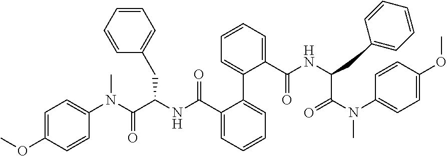

HATU (68 mg, 0.18 mmol) was added to a stirred solution of [1,1′-biphenyl]-2,2′-dicarboxylic acid (21 mg, 0.085 mmol) and an HCl salt of Intermediate 4 (60 mg, 0.19 mmol) in DMF (1 mL) and DIPEA (0.089 mL, 0.51 mmol) and the reaction mixture was stirred at rt for 3 h. The reaction mixture was concentrated, diluted with EtOAc (˜1.5 mL) and washed with water (1 mL) and then brine (1 mL). The organic component was concentrated and the residue was dissolved into DMF, filtered and purified by preparative HPLC to yield the title compound (54 mg). LC-MS retention time=2.37 min; m/z=775.3 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 80

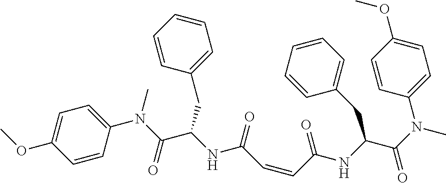

HATU (68 mg, 0.18 mmol) was added to a stirred solution of maleic acid (10 mg, 0.085 mmol) and an HCl salt of Intermediate 4 (60 mg, 0.19 mmol) in DMF (1 mL) and DIPEA (0.09 mL, 0.5 mmol) and stirred at rt for 3 h. The reaction mixture was concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (17.3 mg). LC-MS retention time=2.05 min; m/z=649.4 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, methanol-d₄) δ ppm 7.38-7.18 (m, 8H), 7.02-6.96 (m, 5H), 6.89 (d, J=6.7 Hz, 5H), 6.17 (s, 2H), 4.69 (t, J=7.5 Hz, 2H), 3.78 (s, 7H), 3.17 (s, 7H), 3.03 (dd, J=13.1, 7.6 Hz, 2H), 2.81 (dd, J=13.3, 7.2 Hz, 2H).

### Example 81

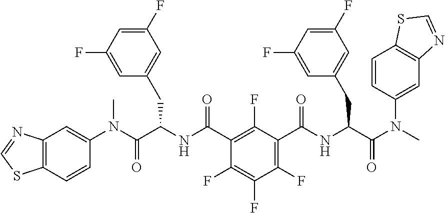 [SMILES: CN(C(=O)[C@H](Cc1cc(F)cc(F)c1)NC(=O)c1c(F)c(F)c(F)c(C(=O)N[C@@H](Cc2cc(F)cc(F)c2)C(=O)N(C)c2ccc3scnc3c2)c1F)c1ccc2scnc2c1]

HATU (50 mg, 0.13 mmol) was added to a stirred solution of 2,4,5,6-tetrafluoroisophthalic acid (15 mg, 0.063 mmol) and an HCl salt of Intermediate 20 (53 mg, 0.14 mmol) in DMF (1 mL) and DIPEA (0.07 mL, 0.4 mmol) and the reaction mixture was stirred at rt for 3 h. The reaction mixture was concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (7.4 mg). LC-MS retention time=2.15 min; m/z=897.3 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 82

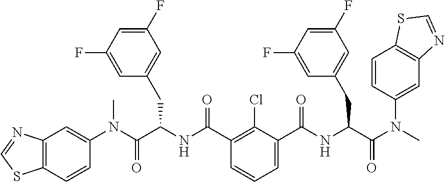 [SMILES: CN(C(=O)[C@H](Cc1cc(F)cc(F)c1)NC(=O)c1cccc(C(=O)N[C@@H](Cc2cc(F)cc(F)c2)C(=O)N(C)c2ccc3scnc3c2)c1Cl)c1ccc2scnc2c1]

HATU (50 mg, 0.13 mmol) was added to a stirred solution of 2-chloroisophthalic acid (13 mg, 0.063 mmol) and an HCl salt of Intermediate 20 (53 mg, 0.14 mmol) in DMF (1 mL) and DIPEA (0.07 mL, 0.4 mmol) and the reaction mixture was stirred at rt for 3 h. The reaction mixture was concentrated, diluted with EtOAc (˜1.5 mL) and washed with water (1 mL) and then brine (1 mL). The organic component was concentrated and the residue was dissolved in MeOH, filtered and purified by preparative HPLC to yield the title compound (24.4 mg). LC-MS retention time=2.04 min; m/z=859.3 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, methanol-d₄) δ ppm 9.38 (s, 2H), 8.22 (d, J=8.5 Hz, 2H), 7.94 (br. s., 1H), 7.47-7.36 (m, 5H), 6.78 (t, J=9.0 Hz, 2H), 6.50 (d, J=6.4 Hz, 4H), 4.61 (s, 3H), 3.33 (s, 6H), 3.12 (dd, J=13.6, 6.6 Hz, 2H), 2.91 (dd, J=13.6, 8.4 Hz, 2H).

### Example 83

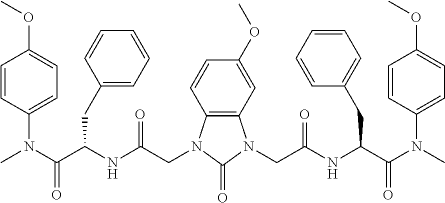 [SMILES: COc1ccc(N(C)C(=O)[C@H](Cc2ccccc2)NC(=O)Cn2c(=O)n(CC(=O)N[C@@H](Cc3ccccc3)C(=O)N(C)c3ccc(OC)cc3)c3cc(OC)ccc32)cc1]

To a solution of an HCl salt of Intermediate 4 (36.1 mg, 0.112 mmol), Intermediate 60 (15 mg, 0.054 mmol) and DIPEA (0.075 mL, 0.43 mmol) in DMF (0.9 mL) was added HATU (44.8 mg, 0.118 mmol). The reaction mixture was stirred at rt for 2 h and then purified by preparative HPLC to afford the title compound as a white solid (33 mg). LC-MS retention time=1.30 min; m/z=813.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 84

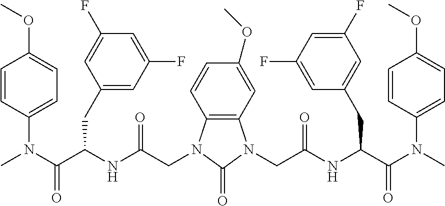 [SMILES: COc1ccc(N(C)C(=O)[C@H](Cc2cc(F)cc(F)c2)NC(=O)Cn2c(=O)n(CC(=O)N[C@@H](Cc3cc(F)cc(F)c3)C(=O)N(C)c3ccc(OC)cc3)c3cc(OC)ccc32)cc1]

To a solution of an HCl salt of Intermediate 13 (40.1 mg, 0.112 mmol), Intermediate 60 (15 mg, 0.054 mmol) and DIPEA (0.075 mL, 0.428 mmol) in DMF (0.9 mL) was added HATU (44.8 mg, 0.118 mmol). The reaction mixture was stirred at rt for 2 h and then purified by preparative HPLC to afford the title compound as a white solid (34 mg). LC-MS retention time=1.34 min; m/z=885.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 85

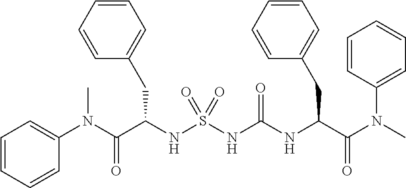 [SMILES: CN(C(=O)[C@H](Cc1ccccc1)NC(=O)NS(=O)(=O)N[C@@H](Cc1ccccc1)C(=O)N(C)c1ccccc1)c1ccccc1]

A solution of 4M HCl (1 mL, 4 mmol) in 1,4-dioxane was added to a stirred solution of Intermediate 26 (77 mg, 0.15 mmol) in THF (1 mL) and the reaction mixture was stirred at rt overnight. The crude reaction mixture was concentrated to dryness, dissolved in DCM (1 mL) and TEA (0.10 mL, 0.71 mmol) and treated with a solution of sulfurisocyanatidic chloride (12 mg, 0.083 mmol) in DCM (˜0.5 mL) and the reaction mixture was stirred rt overnight. The crude reaction mixture was concentrated and the residue was dissolved in MeOH, filtered and purified by preparative HPLC to yield the title compound (14.2 mg). LC-MS retention time=1.98 min; m/z=614.2 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, methanol-d₄) δ ppm 7.45-7.29 (m, 7H), 7.19 (br. s., 6H), 7.03 (br. s., 2H), 6.90 (d, J=3.7 Hz, 5H), 4.62-4.55 (m, 2H), 4.23 (t, J=7.2 Hz, 1H), 3.21 (s, 3H), 3.17 (s, 3H), 2.90 (td, J=13.6, 7.3 Hz, 2H), 2.75-2.66 (m, 2H).

### Example 86

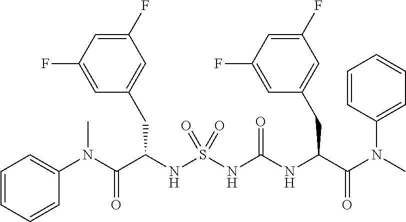 [SMILES: CN(C(=O)[C@H](Cc1cc(F)cc(F)c1)NC(=O)NS(=O)(=O)N[C@@H](Cc1cc(F)cc(F)c1)C(=O)N(C)c1ccccc1)c1ccccc1]

A solution of 4M HCl (1 mL, 4 mmol) in 1,4-dioxane was added to a stirred solution of Intermediate 27 (66 mg, 0.12 mmol) in THF (1 mL) and the reaction mixture was stirred at rt overnight. The crude reaction mixture was concentrated to dryness, dissolved in DCM (1 mL) and TEA (0.10 mL, 0.71 mmol) and treated with a solution of sulfurisocyanatidic chloride (12 mg, 0.083 mmol) in DCM (˜0.5 mL) and the reaction mixture was stirred rt overnight. The crude reaction mixture was concentrated and the residue was dissolved in MeOH, filtered and purified by preparative HPLC to yield the title compound (3.7 mg). LC-MS retention time=2.04 min; m/z=686.2 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 87

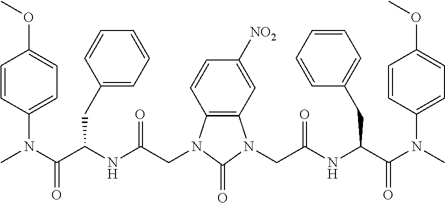 [SMILES: COc1ccc(N(C)C(=O)[C@H](Cc2ccccc2)NC(=O)Cn2c(=O)n(CC(=O)N[C@@H](Cc3ccccc3)C(=O)N(C)c3ccc(OC)cc3)c3cc([N+](=O)[O-])ccc32)cc1]

To a solution of an HCl salt of Intermediate 4 (103 mg, 0.320 mmol), Intermediate 61 (45 mg, 0.15 mmol) and DIPEA (0.21 mL, 1.2 mmol) in DMF (2 mL) was added HATU (128 mg, 0.335 mmol). The reaction mixture was stirred at rt for 2 h and then purified by preparative HPLC to afford the title compound as a white solid (60 mg). LC-MS retention time=1.30 min; m/z=828.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 88

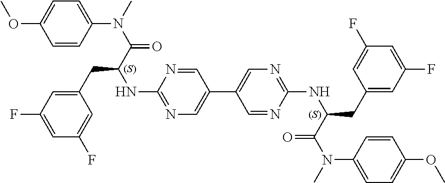

To a stirred solution of Intermediate BB-1 (82 mg, 0.17 mmol) in dioxane (10 mL) and water (3 mL) mixture was added Intermediate BB-2 (100 mg, 0.191 mmol), K₃PO₄ (101 mg, 0.477 mmol) and the reaction mixture was degasified with nitrogen for 10 min. PdCl₂(dppf).CH₂Cl₂ adduct (12.46 mg, 0.015 mmol) was added to the above reaction mixture and heated to reflux for 16 h. The reaction mixture was cooled to RT, diluted with saturated aqueous NH₄Cl solution (50 mL) and extracted with EtOAc (2×50 mL). The combined organic layer was washed with brine (75 mL), dried (Na₂SO₄), filtered and concentrated. The crude product was purified by preparative LC/MS to afford the title product (11 mg) as red liquid. LC-MS retention time=2.31 min; m/z=795.3 [M+H]⁺. Column: Ascentis Express C18 (50×2.1) mm, 2.7 pin; Flow: 1.1 mL/min; Mobile Phase A: 10 mM NH₄OAc in water: ACN (95:5); Mobile Phase B: 10 mM NH₄OAc in water: ACN (5:95); Temperature: 50° C.; 0% B to 100% B over 3 minutes; UV Detection at 220 nm. ¹H NMR (400 MHz, DMSO-d₆) δ 8.53 (s, 4H), 7.55 (d, J=8.0 Hz, 2H), 7.49 (d, J=8.4 Hz, 4H), 7.09 (d, J=9.0 Hz, 4H), 6.98 (dt, J=9.2, 2.4 Hz, 2H), 6.50 (d, J=6.5 Hz, 4H), 4.55 (m, 2H), 3.81 (s, 6H), 3.15 (s, 6H), 2.91-2.86 (m, 4H).

### Example 90

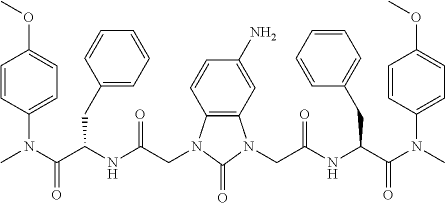 [SMILES: COc1ccc(N(C)C(=O)[C@H](Cc2ccccc2)NC(=O)Cn2c(=O)n(CC(=O)N[C@@H](Cc3ccccc3)C(=O)N(C)c3ccc(OC)cc3)c3cc(N)ccc32)cc1]

To a solution of Example 87 (52 mg, 0.063 mmol) in MeOH (5 mL) was added 10% Pd/C (6.7 mg, 6.3 μmol). The reaction mixture was stirred under a H₂ balloon for 2 h. The reaction mixture was filtered through celite, concentrated in vacuo and then purified by preparative HPLC to afford the title compound as a white solid (24 mg). LC-MS retention time=1.08 min; m/z=798.3 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 92

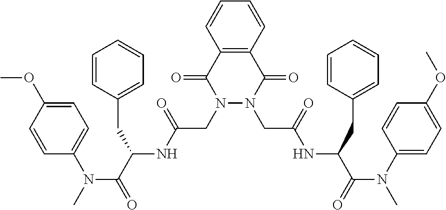 [SMILES: COc1ccc(N(C)C(=O)[C@H](Cc2ccccc2)NC(=O)Cn2c(=O)c3ccccc3c(=O)n2CC(=O)N[C@@H](Cc2ccccc2)C(=O)N(C)c2ccc(OC)cc2)cc1]

To a solution of an HCl salt of Intermediate 4 (24.2 mg, 0.075 mmol), Intermediate 62 (10 mg, 0.036 mmol) and DIPEA (0.05 mL, 0.3 mmol) in DMF (0.9 mL) was added HATU (30 mg, 0.079 mmol). The reaction mixture was stirred at rt for 2 h and then purified by preparative HPLC to afford the title compound as a white solid (23 mg). LC-MS retention time=1.37 min; m/z=811.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 93

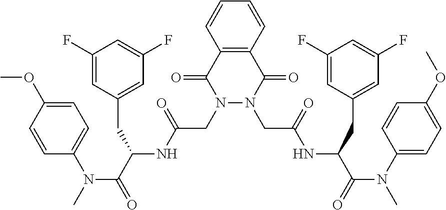 [SMILES: COc1ccc(N(C)C(=O)[C@H](Cc2cc(F)cc(F)c2)NC(=O)Cn2c(=O)c3ccccc3c(=O)n2CC(=O)N[C@@H](Cc2cc(F)cc(F)c2)C(=O)N(C)c2ccc(OC)cc2)cc1]

To a solution of an HCl salt of Intermediate 13 (27 mg, 0.075 mmol), Intermediate 62 (10 mg, 0.036 mmol) and DIPEA (0.05 mL, 0.3 mmol) in DMF (0.9 mL) was added HATU (30 mg, 0.079 mmol). The reaction mixture was stirred at rt for 2 h and then purified by preparative HPLC to afford the title compound as a white solid (23 mg). LC-MS retention time=1.40 min; m/z=883.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 94

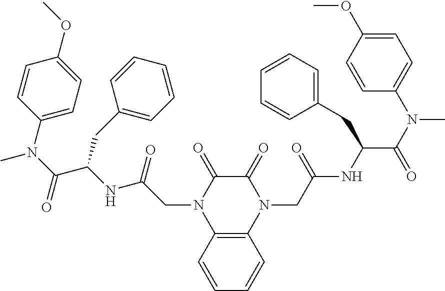

To a solution of an HCl salt of Intermediate 13 (27 mg, 0.075 mmol), Intermediate 62 (10 mg, 0.036 mmol) and DIPEA (0.05 mL, 0.3 mmol) in DMF (0.9 mL) was added HATU (30 mg, 0.079 mmol). The reaction mixture was stirred at rt for 2 h and then purified by preparative HPLC to afford the title compound as a white solid (23 mg). LC-MS retention time=1.99 min; m/z=811.20 [M+H]⁺. (Column: Waters BEH C18, 2.0×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B, 0-100% B over 3 minutes, then a 0.5-minute hold at 100% B; Flow: 1 mL/min; Detection: UV at 220 nm). ¹H NMR (500 MHZ, DMSO-d₆) δ ppm 8.83 (d, J=8.1 Hz, 2H), 7.21 (br. s., 6H), 7.15-7.00 (m, 6H), 6.94 (d, J=8.8 Hz, 4H), 6.86 (br. s., 4H), 6.74 (br. s., 2H), 4.83 (d, J=16.9 Hz, 2H), 4.67 (d, J=17.2 Hz, 2H), 4.47 (d, J=4.4 Hz, 2H), 3.75 (s, 6H), 3.11 (s, 6H), 2.90 (dt, J=8.8, 4.4 Hz, 2H), 2.68 (dd, J=13.2, 9.9 Hz, 2H).

### Example 95

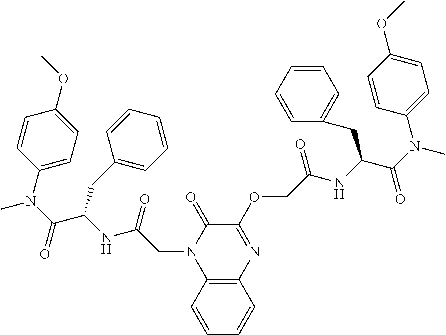

The title compound (4.5 mg) was isolated as a side-product during the synthesis of Example 94 and its structure was tentatively assigned as mono-O-alkylation product aided by proton NMR data. LC-MS retention time=2.09 min; m/z=811.20 [M+H]⁺. (Column: Waters BEH C18, 2.0×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B, 0-100% B over 3 minutes, then a 0.5-minute hold at 100% B; Flow: 1 mL/min; Detection: UV at 220 nm). ¹H NMR (500 MHZ, DMSO-d₆) δ ppm 8.89 (d, J=8.1 Hz, 1H), 8.52 (d, J=7.7 Hz, 1H), 7.43-6.68 (m, 22H), 4.98 (d, J=16.5 Hz, 1H), 4.87-4.65 (m, 3H), 4.49 (dd, J=14.1, 5.3 Hz, 2H), 3.75 (s, 6H), 3.11 (s, 3H), 3.10 (s, 3H), 2.97-2.81 (m, 2H), 2.77-2.63 (m, 2H).

### Example 96

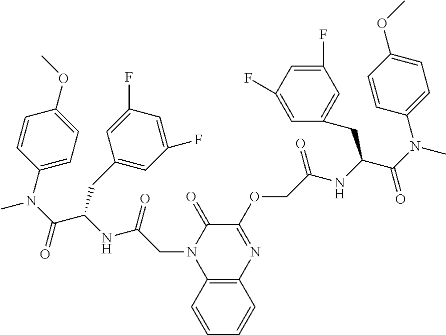

The title compound (4.6 mg) was isolated as a side-product during the synthesis of Example 93 and its structure was tentatively assigned as mono-O-alkylation product aided by proton NMR data. LC-MS retention time=2.19 min; m/z=883.20 [M+H]⁺. (Column: Waters BEH C18, 2.0×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B, 0-100% B over 3 minutes, then a 0.5-minute hold at 100% B; Flow: 1 mL/min; Detection: UV at 220 nm). ¹H NMR (500 MHZ, DMSO-d₆) δ ppm 8.89 (d, J=8.1 Hz, 1H), 8.60 (d, J=8.1 Hz, 1H), 7.44-6.76 (m, 14H), 6.57-6.38 (m, 4H), 4.94 (br. s., 1H), 4.86-4.68 (m, 3H), 4.50 (dt, J=8.3, 4.3 Hz, 2H), 3.75 (s, 6H), 3.14, 3.12 (s, 6H), 2.98-2.84 (m, 2H), 2.80-2.66 (m, 2H).

### Example 97

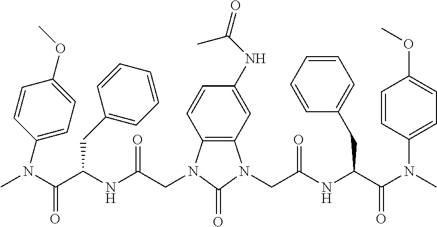 [SMILES: COc1ccc(N(C)C(=O)[C@H](Cc2ccccc2)NC(=O)Cn2c(=O)n(CC(=O)N[C@@H](Cc3ccccc3)C(=O)N(C)c3ccc(OC)cc3)c3cc(NC(C)=O)ccc32)cc1]

To a solution of Example 90 (15 mg, 0.019 mmol) and Hunig's base (10 μl, 0.056 mmol) in DCM (0.5 mL) was added 1 M acetyl chloride in DCM (0.023 mL, 0.023 mmol). The reaction mixture was stirred at rt for 1 h and then concentrated in vacuo. The residue was taken up into DMF and then purified by preparative HPLC to afford the title compound as a white solid (12 mg). LC-MS retention time=1.17 min; m/z=840.3 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 98

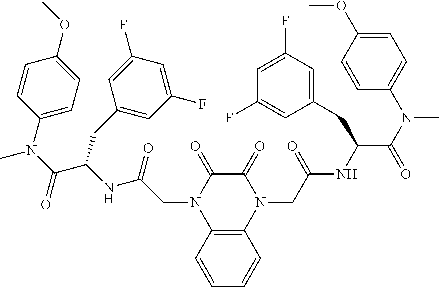

To a solution of an HCl salt of Intermediate 13 (40.4 mg, 0.113 mmol), Intermediate 63 (15 mg, 0.054 mmol) and DIPEA (0.075 mL, 0.43 mmol) in DMF (0.5 mL) was added HATU (45.1 mg, 0.119 mmol). The reaction mixture was stirred at rt for 2 h and then purified by preparative HPLC to afford the title compound as a white solid (28 mg). LC-MS retention time=2.09 min; m/z=883.15 [M+H]⁺. (Column: Waters BEH C18, 2.0×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B, 0-100% B over 3 minutes, then a 0.5-minute hold at 100% B; Flow: 1 mL/min; Detection: UV at 220 nm). ¹H NMR (500 MHZ, methanol-d₄) δ ppm 7.30-7.05 (m, 6H), 7.01-6.85 (m, 6H), 6.76 (t, J=9.0 Hz, 2H), 6.41 (d, J=6.6 Hz, 4H), 4.88 (br. s., 2H), 4.73 (dd, J=9.2, 5.5 Hz, 2H), 3.83-3.72 (m, 6H), 3.26-3.15 (m, 6H), 2.98 (dd, J=13.4, 5.0 Hz, 2H), 2.83-2.69 (m, 2H).

### Example 99

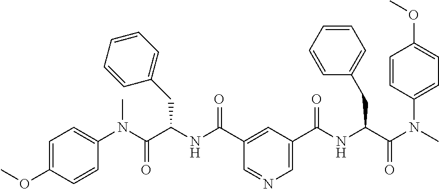 [SMILES: COc1ccc(N(C)C(=O)[C@H](Cc2ccccc2)NC(=O)c2cncc(C(=O)N[C@@H](Cc3ccccc3)C(=O)N(C)c3ccc(OC)cc3)c2)cc1]

HATU (68 mg, 0.18 mmol) was added to a stirred solution of pyridine-3,5-dicarboxylic acid (14 mg, 0.085 mmol) and an HCl salt of Intermediate 4 (60 mg, 0.19 mmol) in DMF (1 mL) and DIPEA (0.09 mL, 0.5 mmol) and the reaction was stirred at rt for 3 h. The reaction mixture was concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (18.8 mg). LC-MS retention time=2.03 min; m/z=700.3 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, methanol-d₄) δ ppm 8.98 (d, J=1.8 Hz, 2H), 8.50 (s, 1H), 7.28-7.20 (m, 6H), 7.15-6.92 (m, 12H), 4.95-4.88 (m, 2H), 3.86 (s, 6H), 3.25 (s, 6H), 3.12 (dd, J=13.6, 6.6 Hz, 2H), 2.94 (dd, J=13.6, 8.4 Hz, 2H).

### Example 100

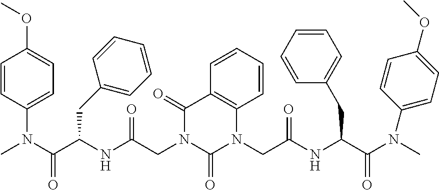 [SMILES: COc1ccc(N(C)C(=O)[C@H](Cc2ccccc2)NC(=O)Cn2c(=O)c3ccccc3n(CC(=O)N[C@@H](Cc3ccccc3)C(=O)N(C)c3ccc(OC)cc3)c2=O)cc1]

To a solution of an HCl salt of Intermediate 4 (24 mg, 0.075 mmol), Intermediate 64 (10 mg, 0.036 mmol) and DIPEA (0.05 mL, 0.3 mmol) in DMF (0.5 mL) was added HATU (30 mg, 0.079 mmol). The reaction mixture was stirred at rt for 2 h and then purified by preparative HPLC to afford the title compound as a white solid (22 mg). LC-MS retention time=1.29 min; m/z=811.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 101

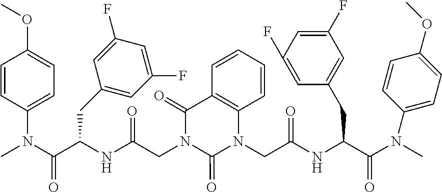 [SMILES: COc1ccc(N(C)C(=O)[C@H](Cc2cc(F)cc(F)c2)NC(=O)Cn2c(=O)c3ccccc3n(CC(=O)N[C@@H](Cc3cc(F)cc(F)c3)C(=O)N(C)c3ccc(OC)cc3)c2=O)cc1]

To a solution of an HCl salt of Intermediate 13 (27 mg, 0.075 mmol), Intermediate 64 (10 mg, 0.036 mmol) and DIPEA (0.05 mL, 0.3 mmol) in DMF (0.5 mL) was added HATU (30 mg, 0.079 mmol). The reaction mixture was stirred at rt for 2 h and then purified by preparative HPLC to afford the title compound as a white solid (22 mg). LC-MS retention time=1.34 min; m/z=883.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 102

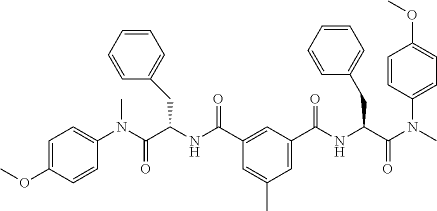 [SMILES: COc1ccc(N(C)C(=O)[C@H](Cc2ccccc2)NC(=O)c2cc(C)cc(C(=O)N[C@@H](Cc3ccccc3)C(=O)N(C)c3ccc(OC)cc3)c2)cc1]

HATU (68 mg, 0.18 mmol) was added to a stirred solution of 5-methylisophthalic acid (15 mg, 0.085 mmol) and an HCl salt of Intermediate 4 (60 mg, 0.19 mmol) in DMF (1 mL) and DIPEA (0.09 mL, 0.5 mmol) and the reaction was stirred at rt for 3 h. The reaction mixture was concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (21.6 mg). LC-MS retention time=2.22 min; m/z=713.3 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, methanol-d₄) δ ppm 7.96 (s, 1H), 7.72 (s, 2H), 7.27-7.20 (m, 6H), 7.12-6.93 (m, 12H), 4.94-4.85 (m, 2H), 3.85 (s, 6H), 3.24 (s, 6H), 3.10 (dd, J=13.3, 6.6 Hz, 2H), 2.94 (dd, J=13.3, 8.4 Hz, 2H), 2.43 (s, 3H).

### Example 103

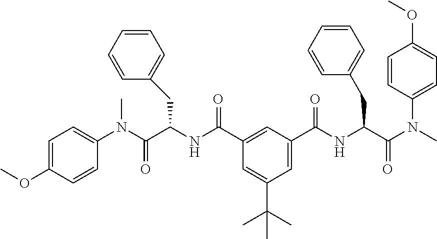 [SMILES: COc1ccc(N(C)C(=O)[C@H](Cc2ccccc2)NC(=O)c2cc(C(=O)N[C@@H](Cc3ccccc3)C(=O)N(C)c3ccc(OC)cc3)cc(C(C)(C)C)c2)cc1]

HATU (68 mg, 0.18 mmol) was added to a stirred solution of 5-(tert-butyl)isophthalic acid (19 mg, 0.085 mmol) and an HCl salt of Intermediate 4 (60 mg, 0.19 mmol) in DMF (1 mL) and DIPEA (0.09 mL, 0.5 mmol) and the reaction was stirred at rt for 3 h. The reaction mixture was concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (43.8 mg). LC-MS retention time=2.49 min; m/z=755.2 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, methanol-d₄) δ ppm 8.02 (s, 1H), 7.93 (s, 2H), 7.26-7.19 (m, 6H), 7.14-6.95 (m, 12H), 4.96 (t, J=7.3 Hz, 2H), 3.85 (s, 6H), 3.29 (s, 6H), 3.14-3.08 (m, 2H), 3.04-2.96 (m, 2H), 1.35 (s, 9H).

### Example 104

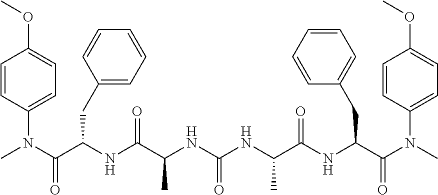 [SMILES: COc1ccc(N(C)C(=O)[C@H](Cc2ccccc2)NC(=O)[C@H](C)NC(=O)N[C@@H](C)C(=O)N[C@@H](Cc2ccccc2)C(=O)N(C)c2ccc(OC)cc2)cc1]

To a solution of an HCl salt of Intermediate 4 (26 mg, 0.082 mmol), Intermediate 65 (8 mg, 0.04 mmol) and DIPEA (0.055 mL, 0.31 mmol) in DMF (0.9 mL) was added HATU (33 mg, 0.086 mmol). The reaction mixture was stirred at rt for 2 h and then purified by preparative HPLC to afford the title compound as a white solid (16 mg). LC-MS retention time=1.17 min; m/z=737.3 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm). ¹H NMR (500 MHZ, methanol-d₄) δ ppm 7.23 (d, J=2.6 Hz, 6H), 7.06-6.66 (m, 12H), 4.73-4.53 (m, 4H), 4.17 (d, J=7.0 Hz, 2H), 3.89-3.77 (m, 6H), 3.16 (s, 6H), 2.97 (dd, J=13.2, 7.3 Hz, 2H), 2.75 (dd, J=13.2, 7.3 Hz, 2H), 1.28 (d, J=7.3 Hz, 6H).

### Example 105

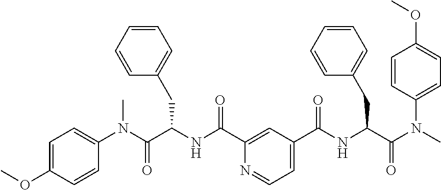 [SMILES: COc1ccc(N(C)C(=O)[C@H](Cc2ccccc2)NC(=O)c2ccnc(C(=O)N[C@@H](Cc3ccccc3)C(=O)N(C)c3ccc(OC)cc3)c2)cc1]

HATU (68 mg, 0.18 mmol) was added to a stirred solution of pyridine-2,4-dicarboxylic acid (14 mg, 0.085 mmol) and an HCl salt of Intermediate 4 (60 mg, 0.19 mmol) in DMF (1 mL) and DIPEA (0.09 mL, 0.5 mmol) and the reaction mixture was stirred at rt for 3 h. The reaction mixture was concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (11.9 mg). LC-MS retention time=2.21 min; m/z=700.1 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 106

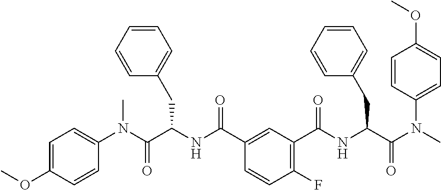 [SMILES: COc1ccc(N(C)C(=O)[C@H](Cc2ccccc2)NC(=O)c2ccc(F)c(C(=O)N[C@@H](Cc3ccccc3)C(=O)N(C)c3ccc(OC)cc3)c2)cc1]

HATU (68 mg, 0.18 mmol) was added to a stirred solution of 4-fluoroisophthalic acid (16 mg, 0.085 mmol) and an HCl salt of Intermediate 4 (60 mg, 0.19 mmol) in DMF (1 mL) and DIPEA (0.09 mL, 0.5 mmol) and the reaction mixture was stirred at rt for 3 h. The reaction mixture was concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (38.3 mg). LC-MS retention time=2.23 min; m/z=717.2 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, methanol-d₄) δ ppm 8.09 (d, J=6.7 Hz, 1H), 7.93 (d, J=3.1 Hz, 1H), 7.42-7.20 (m, 8H), 7.13-6.92 (m, 11H), 4.95-4.85 (m, 2H), 3.86 (s, 6H), 3.22 (s, 6H), 3.12-3.05 (m, 2H), 2.95-2.85 (m, 2H).

### Example 107

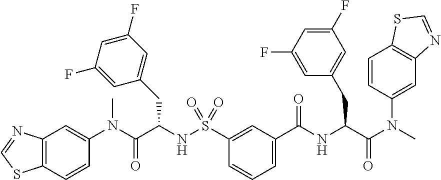 [SMILES: CN(C(=O)[C@H](Cc1cc(F)cc(F)c1)NC(=O)c1cccc(S(=O)(=O)N[C@@H](Cc2cc(F)cc(F)c2)C(=O)N(C)c2ccc3scnc3c2)c1)c1ccc2scnc2c1]

TEA (0.055 mL, 0.40 mmol) was added to a solution of an HCl salt of Intermediate 20 (61 mg, 0.16 mmol) and 3-(chlorosulfonyl)benzoyl chloride (19 mg, 0.079 mmol) in DCM (1 mL) and the reaction mixture was sealed and stirred at rt for 3 h. The crude reaction mixture was concentrated and the residue was dissolved into MeOH, filtered and purified by preparative HPLC to yield the title compound (38.6 mg). LC-MS retention time=2.01 min; m/z=861.3 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 108

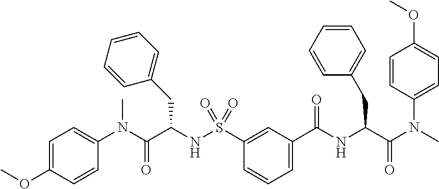 [SMILES: COc1ccc(N(C)C(=O)[C@H](Cc2ccccc2)NC(=O)c2cccc(S(=O)(=O)N[C@@H](Cc3ccccc3)C(=O)N(C)c3ccc(OC)cc3)c2)cc1]

TEA (0.055 mL, 0.40 mmol) was added to a solution of an HCl salt of Intermediate 4 (51 mg, 0.16 mmol) and 3-(chlorosulfonyl)benzoyl chloride (19 mg, 0.079 mmol) in DCM (1 mL) and the reaction mixture was sealed and stirred at rt for 3 h. The crude reaction mixture was concentrated and the residue was dissolved into MeOH, filtered and purified by preparative HPLC to yield the title compound (37.5 mg). LC-MS retention time=2.14 min; m/z=735.3 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, methanol-d₄) δ ppm 8.04-7.99 (m, 1H), 7.91 (d, J=7.6 Hz, 1H), 7.61 (d, J=8.2 Hz, 1H), 7.44 (t, J=7.9 Hz, 1H), 7.25-7.17 (m, 4H), 7.15-6.90 (m, 8H), 6.85-6.65 (m, 6H), 3.91-3.85 (m, 4H), 3.76 (s, 3H), 3.25 (s, 3H), 3.13 (dd, J=13.6, 6.3 Hz, 1H), 3.04 (s, 3H), 2.95 (dd, J=13.4, 8.9 Hz, 1H), 2.91-2.86 (m, 2H), 2.59 (dd, J=13.4, 8.9 Hz, 1H).

### Example 109

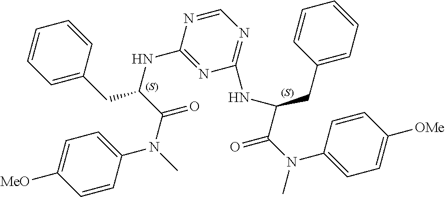

To a stirred solution of Intermediate BB-3 (100 mg, 0.15 mmol) in EtOH (10 mL) was added 10% Pd/C (10 mg, 0.01 mmol) followed by ammonium formate (28 mg, 0.44 mmol) and the reaction mixture was stirred at 100° C. for 1 h. The reaction mixture was filtered through Celite and the filtrate concentrated under reduced pressure. The crude product was purified by preparative HPLC to afford the title product (3 mg) as an off white solid. LCMS retention time=1.76 min; m/z=646.4 [M+H]⁺. Column: Ascentis Express C18 (50×2.1) mm, 2.7 μm; Flow: 1.1 mL/min; Mobile Phase A: 10 mM NH₄OAc in water: ACN (95:5); Mobile Phase B: 10 mM NH₄OAc in water: ACN (5:95); Temperature: 50° C.; 0% B to 100% B over 3 minutes; UV Detection at 220 nm.

### Example 110

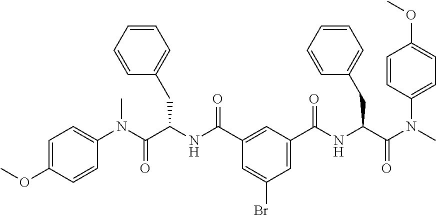 [SMILES: COc1ccc(N(C)C(=O)[C@H](Cc2ccccc2)NC(=O)c2cc(Br)cc(C(=O)N[C@@H](Cc3ccccc3)C(=O)N(C)c3ccc(OC)cc3)c2)cc1]

A solution of 1M aqueous LiOH (0.75 mL, 0.75 mmol) was added to a stirred solution of dimethyl 5-bromoisophthalate (97 mg, 0.36 mmol) in THF (1 mL) and the reaction mixture was stirred at rt overnight and then concentrated to dryness. The crude residue was dissolved into DMF (1 mL) and 4M HCl in 1,4-dioxane (0.19 mL, 0.76 mmol) and stirred until clear. A portion (0.1 mmol, 235 uL) of this crude solution was combined with a solution of an HCl salt of Intermediate 4 (67.4 mg, 210 μmol) in DMF (0.83 mL) and DIPEA (0.07 mL, 400 μmol) and the crude reaction mixture was then treated with HATU (84 mg, 220 μmol) and stirred at rt for 3 h. The reaction mixture was concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (32.8 mg). LC-MS retention time=2.36 min; m/z=777.3 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, DMSO-d₆) δ ppm 8.97 (d, J=7.7 Hz, 2H), 8.26 (s, 1H), 8.13 (s, 2H), 7.25 (d, J=6.2 Hz, 4H), 7.20-7.11 (m, 6H), 7.03 (d, J=8.8 Hz, 4H), 6.89 (d, J=7.0 Hz, 4H), 4.70-4.63 (m, 2H), 3.81 (s, 6H), 3.15 (s, 6H), 2.98-2.86 (m, 4H).

### Example 111

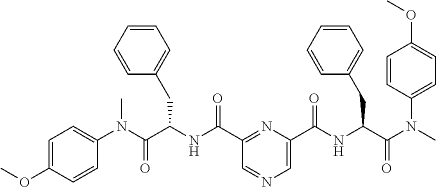 [SMILES: COc1ccc(N(C)C(=O)[C@H](Cc2ccccc2)NC(=O)c2cncc(C(=O)N[C@@H](Cc3ccccc3)C(=O)N(C)c3ccc(OC)cc3)n2)cc1]

A solution of 1M aqueous LiOH (0.75 mL, 0.75 mmol) was added to a stirred solution of dimethyl pyrazine-2,6-dicarboxylate (70 mg, 0.36 mmol) in THF (1 mL) and the reaction mixture was stirred at rt overnight and then concentrated to dryness. The crude residue was dissolved into DMF (1 mL) and 4M HCl in 1,4-dioxane (0.19 mL, 0.76 mmol) and stirred until clear. A portion (0.1 mmol, 235 uL) of this crude solution was combined with a solution of an HCl salt of Intermediate 4 (67.4 mg, 210 μmol) in DMF (0.83 mL) and DIPEA (0.07 mL, 400 μmol) and the crude reaction mixture was then treated with HATU (84 mg, 220 μmol) and stirred at rt for 3 h. The reaction mixture was concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (43 mg). LC-MS retention time=2.23 min; m/z=701.4 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, methanol-d₄) δ ppm 9.31 (s, 2H), 7.46-7.16 (m, 7H), 7.11-6.87 (m, 11H), 5.00 (t, J=7.2 Hz, 2H), 4.90 (br. s., 2H), 3.86 (s, 6H), 3.28-3.19 (m, 8H), 3.05 (dd, J=13.2, 8.1 Hz, 2H).

### Example 112

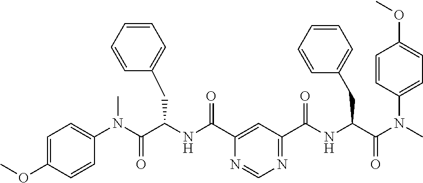 [SMILES: COc1ccc(N(C)C(=O)[C@H](Cc2ccccc2)NC(=O)c2cc(C(=O)N[C@@H](Cc3ccccc3)C(=O)N(C)c3ccc(OC)cc3)ncn2)cc1]

A solution of 1 M aqueous LiOH (0.75 mL, 0.75 mmol) was added to a stirred solution of dimethyl pyrimidine-4,6-dicarboxylic acid (70 mg, 360 μmol) in THF (1 mL) and the reaction mixture was stirred at rt overnight and then concentrated to dryness. The crude residue was dissolved into DMF (1 mL) and 4M HCl in 1,4-dioxane (0.19 mL, 0.76 mmol) and stirred until the mixture became clear. A portion (0.1 mmol, 235 uL) of this crude solution was combined with a solution of an HCl salt of Intermediate 4 (67.4 mg, 210 μmol) in DMF (0.83 mL) and DIPEA (0.07 mL, 400 μmol) and the crude reaction mixture was then treated with HATU (84 mg, 220 μmol) and stirred at rt for 3 h. The reaction mixture was concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (22 mg). LC-MS retention time=2.21 min; m/z=701.4 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, methanol-d₄) δ ppm 9.39 (s, 1H), 8.45 (s, 1H), 7.41-7.19 (m, 7H), 7.08-6.84 (m, 11H), 4.93 (t, J=7.2 Hz, 2H), 4.90-4.85 (m, 2H), 3.85 (s, 6H), 3.22 (s, 6H), 3.11 (dd, J=13.2, 7.0 Hz, 2H), 2.93 (dd, J=13.4, 7.5 Hz, 2H).

### Example 113 and Example 114

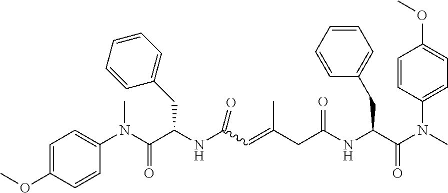 [SMILES: COc1ccc(N(C)C(=O)[C@H](Cc2ccccc2)NC(=O)C=C(C)CC(=O)N[C@@H](Cc2ccccc2)C(=O)N(C)c2ccc(OC)cc2)cc1]

A solution of 1M aqueous LiOH (0.76 mL, 0.76 mmol) was added to a stirred solution of (E)-dimethyl 3-methylpent-2-enedioate (62 mg, 0.36 mmol) in THF and the reaction mixture was stirred at rt for 2 d and then concentrated to dryness. The crude residue was dissolved into DMF (1 mL) and 4M HCl in 1,4-dioxane (0.19 mL, 0.76 mmol) and stirred until clear. A portion (0.1 mmol, 235 uL) of this crude solution was combined with a solution of an HCl salt of Intermediate 4 (67.4 mg, 210 μmol) in DMF (0.83 mL) and DIPEA (0.07 mL, 400 μmol) and the crude reaction mixture was then treated with HATU (84 mg, 220 μmol) and stirred at rt for 3 h. The reaction mixture was concentrated, diluted with EtOAc (˜1.5 mL) and washed with water (1 mL) and then brine (1 mL). The organic component was concentrated and the residue was dissolved into MeOH, filtered and purified by preparative HPLC to yield two isomers. Olefin stereochemistry was not determined:

The first eluting isomer of the title compound (12.4 mg). LC-MS retention time=1.99 min; m/z=677.4 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, methanol-d₄) δ ppm 7.25-7.18 (m, 6H), 7.07-6.77 (d, J=4.9 Hz, 12H), 5.74 (s, 1H), 4.71 (t, J=7.5 Hz, 1H), 4.66 (t, J=7.5 Hz, 1H), 3.84 (s, 6H), 3.19 (s, 3H), 3.18 (s, 3H), 3.02-2.94 (m, 4H), 2.75 (dd, J=13.4, 8.2 Hz, 2H), 1.87 (s, 3H). The second eluting isomer of the title compound (5.0 mg). LC-MS retention time=2.11 min; m/z=677.4 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, methanol-d₄) δ ppm 7.26-6.89 (m, 18H), 6.83 (d, J=7.0 Hz, 2H), 5.78 (s, 1H), 4.71 (t, J=7.3 Hz, 1H), 4.66-4.60 (m, 3H), 3.44 (d, J=12.2 Hz, 1H), 3.22 (s, 3H), 3.21 (s, 3H), 3.17 (d, J=11.9 Hz, 2H), 3.01 (dd, J=13.1, 6.7 Hz, 1H), 2.95 (dd, J=13.3, 6.3 Hz, 1H), 2.81-2.76 (m, 1H), 2.65 (dd, J=13.4, 8.5 Hz, 1H), 1.76 (s, 3H).

### Example 115

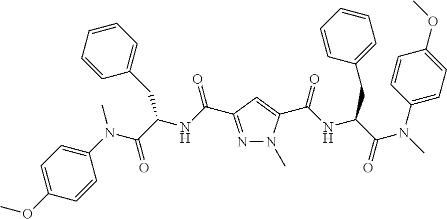 [SMILES: COc1ccc(N(C)C(=O)[C@H](Cc2ccccc2)NC(=O)c2cc(C(=O)N[C@@H](Cc3ccccc3)C(=O)N(C)c3ccc(OC)cc3)n(C)n2)cc1]

A solution of 1M aqueous LiOH (0.76 mL, 0.76 mmol) was added to a stirred solution of 3-(ethoxycarbonyl)-1-methyl-1H-pyrazole-5-carboxylic acid (71 mg, 0.36 mmol) in THF and the reaction mixture was stirred at rt for 2 d and then concentrated to dryness. The crude residue was dissolved into DMF (1 mL) and 4M HCl in 1,4-dioxane (0.19 mL, 0.76 mmol) and stirred until clear. A portion (0.1 mmol, 235 uL) of this crude solution was combined with a solution of an HCl salt of Intermediate 4 (67.4 mg, 210 μmol) in DMF (0.83 mL) and DIPEA (0.07 mL, 400 μmol) and the crude reaction mixture was then treated with HATU (84 mg, 220 μmol) and stirred at rt for 3 h. The reaction mixture was concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (37.7 mg). LC-MS retention time=2.14 min; m/z=703.3 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, methanol-d₄) δ ppm 7.41-7.17 (m, 7H), 7.11 (s, 1H), 7.07-6.84 (m, 11H), 4.90-4.81 (m, 3H), 4.04 (s, 3H), 3.86 (s, 2H), 3.85 (s, 3H), 3.23 (s, 3H), 3.21 (s, 3H), 3.09-3.02 (m, 2H), 2.91-2.83 (m, 2H).

### Example 116

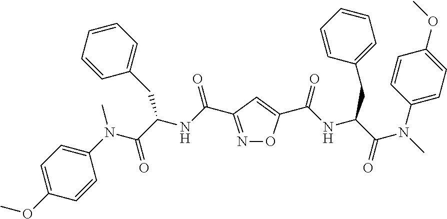 [SMILES: COc1ccc(N(C)C(=O)[C@H](Cc2ccccc2)NC(=O)c2cc(C(=O)N[C@@H](Cc3ccccc3)C(=O)N(C)c3ccc(OC)cc3)on2)cc1]

A solution of 1M aqueous LiOH (0.76 mL, 0.76 mmol) was added to a stirred solution of 3-(ethoxycarbonyl)isoxazole-5-carboxylic acid (66.7 mg, 0.360 mmol) in THF and the reaction mixture was stirred at rt for 2 d and then concentrated to dryness. The crude residue was dissolved into DMF (1 mL) and 4M HCl in 1,4-dioxane (0.19 mL, 0.76 mmol) and stirred until clear. A portion (0.1 mmol, 235 uL) of this crude solution was combined with a solution of an HCl salt of Intermediate 4 (67.4 mg, 210 μmol) in DMF (0.83 mL) and DIPEA (0.07 mL, 400 μmol) and the crude reaction mixture was then treated with HATU (84 mg, 220 μmol) and stirred at rt for 3 h. The reaction mixture was concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (24.9 mg). LC-MS retention time=2.09 min; m/z=690.3 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 117

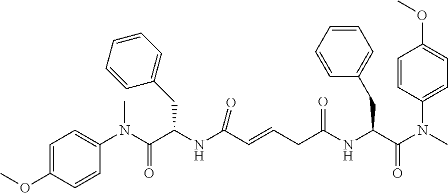 [SMILES: COc1ccc(N(C)C(=O)[C@H](Cc2ccccc2)NC(=O)/C=C/CC(=O)N[C@@H](Cc2ccccc2)C(=O)N(C)c2ccc(OC)cc2)cc1]

A solution of 1M aqueous LiOH (0.76 mL, 0.76 mmol) was added to a stirred solution of (E)-dimethyl pent-2-enedioate (57 mg, 0.36 mmol) in THF and the reaction mixture was stirred at rt for 2 d and then concentrated to dryness. The crude residue was dissolved into DMF (1 mL) and 4M HCl in 1,4-dioxane (0.19 mL, 0.76 mmol) and stirred until clear. A portion (0.1 mmol, 235 uL) of this crude solution was combined with a solution of an HCl salt of Intermediate 4 (67.4 mg, 210 μmol) in DMF (0.83 mL) and DIPEA (0.07 mL, 400 μmol) and the crude reaction mixture was then treated with HATU (84 mg, 220 μmol) and stirred at rt for 3 h. The reaction mixture was concentrated, diluted with EtOAc (˜1.5 mL) and washed with water (1 mL) and then brine (1 mL). The organic component was concentrated and the residue was dissolved into MeOH, filtered and purified by preparative HPLC to yield the title compound (21.9 mg). LC-MS retention time=1.91 min; m/z=663.4 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, methanol-d₄) δ ppm 7.39-7.16 (m, 7H), 7.09-6.78 (m, 11H), 6.73-6.65 (m, 1H), 6.02 (d, J=15.4 Hz, 1H), 4.73 (t, J=7.3 Hz, 1H), 4.64 (t, J=7.3 Hz, 1H), 3.83 (s, 6H), 3.17 (s, 6H), 3.08 (d, J=7.3 Hz, 2H), 2.99 (td, J=12.9, 7.2 Hz, 2H), 2.81-2.70 (m, 2H).

### Example 118

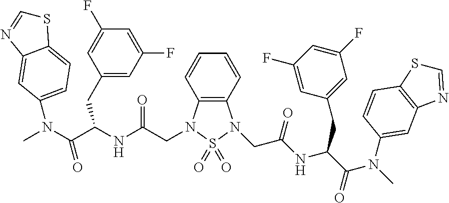 [SMILES: CN(C(=O)[C@H](Cc1cc(F)cc(F)c1)NC(=O)CN1c2ccccc2N(CC(=O)N[C@@H](Cc2cc(F)cc(F)c2)C(=O)N(C)c2ccc3scnc3c2)S1(=O)=O)c1ccc2scnc2c1]

To a solution of an HCl salt of Intermediate 20 (46 mg, 0.11 mmol), Intermediate 51 (15 mg, 0.052 mmol) and DIPEA (0.07 mL, 0.4 mmol) in DMF (0.5 mL) was added HATU (42 mg, 0.11 mmol). The reaction mixture was stirred at rt for 2 h and then purified by preparative HPLC to afford the title compound as a white solid (17 mg). LC-MS retention time=1.28 min; m/z=945.3 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm). ¹H NMR (500 MHZ, methanol-d₄) δ ppm 9.45-9.29 (m, 2H), 8.15 (d, J=8.4 Hz, 2H), 7.88 (br. s., 2H), 7.35 (br. s., 2H), 7.00 (br. s., 2H), 6.76-6.62 (m, 4H), 6.38 (d, J=6.2 Hz, 4H), 4.74 (t, J=7.0 Hz, 2H), 4.40 (s, 4H), 3.33 (s, 6H), 3.07-2.96 (m, 2H), 2.80 (dd, J=13.6, 8.1 Hz, 2H).

### Example 119

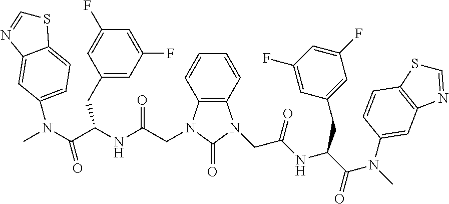 [SMILES: CN(C(=O)[C@H](Cc1cc(F)cc(F)c1)NC(=O)Cn1c(=O)n(CC(=O)N[C@@H](Cc2cc(F)cc(F)c2)C(=O)N(C)c2ccc3scnc3c2)c2ccccc21)c1ccc2scnc2c1]

To a solution of an HCl salt of Intermediate 20 (46 mg, 0.11 mmol), Intermediate 52 (13 mg, 0.052 mmol) and DIPEA (0.07 mL, 0.4 mmol) in DMF (0.5 mL) was added HATU (41.5 mg, 0.109 mmol). The reaction mixture was stirred at rt for 2 h and purified preparative HPLC to afford the title compound as a white solid (27 mg). LC-MS retention time=1.96 min; m/z=909.0 [M+H]⁺. (Column: Waters BEH C18, 2.0×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B, 0-100% B over 3 minutes, then a 0.5-minute hold at 100% B; Flow: 1 mL/min; Detection: UV at 220 nm). ¹H NMR (500 MHZ, methanol-d₄) δ ppm 9.32 (s, 2H), 8.11 (d, J=8.4 Hz, 2H), 7.85 (br. s., 2H), 7.33 (br. s., 2H), 7.08 (d, J=2.6 Hz, 2H), 6.90 (d, J=5.1 Hz, 2H), 6.74 (t, J=9.2 Hz, 2H), 6.42 (d, J=6.6 Hz, 4H), 4.73 (t, J=7.2 Hz, 2H), 4.55 (s, 4H), 3.33 (s, 6H), 3.05 (dd, J=13.8, 6.4 Hz, 2H), 2.84 (dd, J=13.4, 8.3 Hz, 2H).

### Example 120

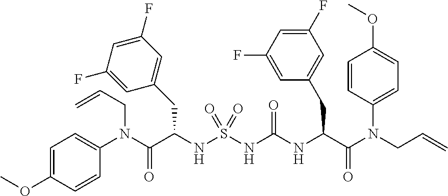 [SMILES: C=CCN(C(=O)[C@H](Cc1cc(F)cc(F)c1)NC(=O)NS(=O)(=O)N[C@@H](Cc1cc(F)cc(F)c1)C(=O)N(CC=C)c1ccc(OC)cc1)c1ccc(OC)cc1]

A solution of sulfurisocyanatidic chloride (23 mg, 0.17 mmol) in DCM (0.20 mL) was added dropwise to a stirred solution of an HCl salt of Intermediate 32 (120 mg, 0.28 mmol) in DCM (1.5 mL) and TEA (0.12 mL, 0.83 mmol) at 0° C. and then the reaction solution was allowed to warm to rt and was stirred for 3 h. The crude reaction mixture was concentrated and the residue was dissolved into MeOH, filtered and purified by preparative HPLC to yield the title compound (52.4 mg). LC-MS retention time=2.25 min; m/z=798.1 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, methanol-d₄) δ ppm 7.26-6.71 (m, 10H), 6.55 (d, J=6.4 Hz, 2H), 6.46 (d, J=6.1 Hz, 2H), 5.86-5.70 (m, 2H), 5.13-5.00 (m, 4H), 4.29-4.13 (m, 6H), 3.88-3.78 (m, 6H), 3.00-2.88 (m, 2H), 2.80-2.65 (m, 2H).

### Example 121

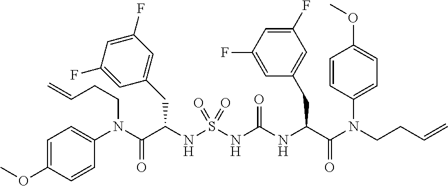 [SMILES: C=CCCN(C(=O)[C@H](Cc1cc(F)cc(F)c1)NC(=O)NS(=O)(=O)N[C@@H](Cc1cc(F)cc(F)c1)C(=O)N(CCC=C)c1ccc(OC)cc1)c1ccc(OC)cc1]

A solution of sulfurisocyanatidic chloride (23 mg, 0.17 mmol) in DCM (0.20 mL) was added dropwise to a stirred solution of an HCl salt of Intermediate 33 (125 mg, 0.28 mmol) in DCM (1.5 mL) and TEA (0.12 mL, 0.83 mmol) at 0° C. and then the reaction solution was allowed to warm to rt and was stirred for 3 h. The crude reaction mixture was concentrated and the residue was dissolved into MeOH, filtered and purified by preparative HPLC to yield the title compound (57.9 mg). LC-MS retention time=2.43 min; m/z=826.2 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, methanol-d₄) δ ppm 7.52-6.66 (m, 10H), 6.54 (d, J=6.7 Hz, 2H), 6.47 (d, J=7.0 Hz, 2H), 5.80-5.68 (m, 2H), 5.09-4.98 (m, 4H), 4.59-4.54 (m, 1H), 4.21 (t, J=6.9 Hz, 1H), 3.91-3.79 (m, 6H), 3.77-3.56 (m, 4H), 2.98-2.86 (m, 2H), 2.78-2.64 (m, 2H), 2.23 (dd, J=14.8, 7.2 Hz, 4H).

### Example 122

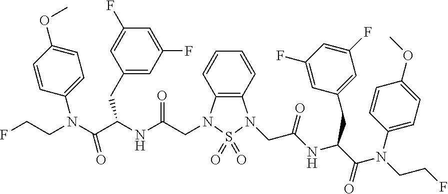 [SMILES: COc1ccc(N(CCF)C(=O)[C@H](Cc2cc(F)cc(F)c2)NC(=O)CN2c3ccccc3N(CC(=O)N[C@@H](Cc3cc(F)cc(F)c3)C(=O)N(CCF)c3ccc(OC)cc3)S2(=O)=O)cc1]

To a solution of an HCl salt of Intermediate 67 (43 mg, 0.11 mmol), Intermediate 51 (15 mg, 0.052 mmol) and DIPEA (0.07 mL, 0.4 mmol) in DMF (0.5 mL) was added HATU (42 mg, 0.11 mmol). The reaction mixture was stirred at rt for 2 h and then purified by preparative HPLC to afford the title compound as a white solid (31 mg). LC-MS retention time=1.28 min; m/z=955.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 123

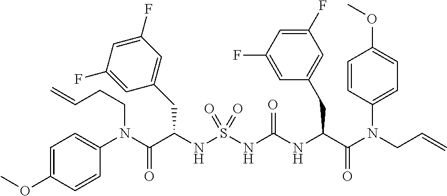 [SMILES: C=CCCN(C(=O)[C@H](Cc1cc(F)cc(F)c1)NS(=O)(=O)NC(=O)N[C@@H](Cc1cc(F)cc(F)c1)C(=O)N(CC=C)c1ccc(OC)cc1)c1ccc(OC)cc1]

A solution of an HCl salt of Intermediate 32 (60 mg, 0.14 mmol) in DCM (0.7 mL) was added dropwise to a stirred solution of sulfurisocyanatidic chloride (12 mg, 0.083 mmol) in DCM (0.5 mL) at 0° C. The reaction mixture was allowed to warm to rt, stirred 30 min, treated with TEA (1 eq) and stirred 1 h. Additional sulfurisocyanatidic chloride (12 mg, 0.083 mmol) in DCM (0.3 mL) was added dropwise and the reaction mixture was stirred 30 min before being treated with additional with TEA (0.12 mL, 0.83 mmol). The reaction mixture was then treated with an HCl salt of Intermediate 33 (62 mg, 0.11 mmol) in DCM (0.5 mL) and stirred at rt for 3 h. The crude reaction mixture was concentrated and the residue was dissolved into MeOH, filtered and purified by preparative HPLC to yield the title compound (23.9 mg). LC-MS retention time=2.30 min; m/z=812.3 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 124

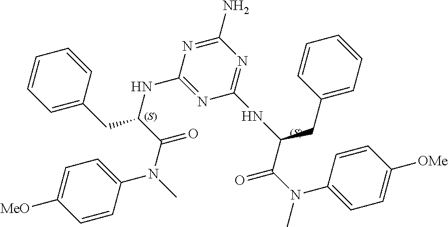

To a stirred solution of Intermediate BB-4 (100 mg, 0.24 mmol) in dioxane (10 mL) was added Intermediate 4 (83 mg, 0.29 mmol) followed by DIPEA (0.13 mL, 0.72 mmol) and the reaction mixture was stirred at 80° C. for 24 h. The reaction mixture was diluted with water (20 mL) and extracted with dichloromethane (3×20 mL). The combined organic layer was washed with water (20 mL), brine (20 mL), dried (Na₂SO₄), filtered and concentrated. The crude product was purified by preparative HPLC to afford the title compound (4 mg) as an off white solid. LCMS retention time=1.99 min; m/z=661.4 [M+H]⁺. Column: Ascentis Express C18 (50×2.1) mm, 2.7 μm; Flow: 1.1 mL/min; Mobile Phase A: 10 mM NH₄OAc in water: ACN (95:5); Mobile Phase B: 10 mM NH₄OAc in water: ACN (5:95); Temperature: 50° C.; 0% B to 100% B over 3 minutes; UV Detection at 220 nm.

### Example 125

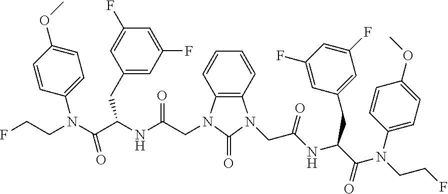 [SMILES: COc1ccc(N(CCF)C(=O)[C@H](Cc2cc(F)cc(F)c2)NC(=O)Cn2c(=O)n(CC(=O)N[C@@H](Cc3cc(F)cc(F)c3)C(=O)N(CCF)c3ccc(OC)cc3)c3ccccc32)cc1]

To a solution of an HCl salt of Intermediate 67 (42.4 mg, 0.109 mmol), Intermediate 52 (13 mg, 0.052 mmol) and DIPEA (0.07 mL, 0.4 mmol) in DMF (0.5 mL) was added HATU (41.5 mg, 0.109 mmol). The reaction mixture was stirred at rt for 2 h and purified preparative HPLC to afford the title compound as a white solid (39 mg). LC-MS retention time=1.36 min; m/z=919.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 126

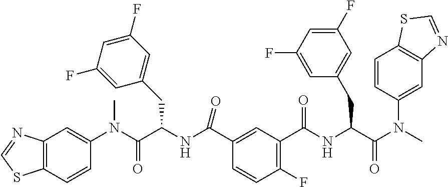 [SMILES: CN(C(=O)[C@H](Cc1cc(F)cc(F)c1)NC(=O)c1ccc(F)c(C(=O)N[C@@H](Cc2cc(F)cc(F)c2)C(=O)N(C)c2ccc3scnc3c2)c1)c1ccc2scnc2c1]

HATU (38 mg, 0.10 mmol) was added to a stirred solution of 4-fluoroisophthalic acid (8.8 mg, 0.048 mmol) and an HCl salt of Intermediate 20 (40.4 mg, 0.105 mmol) in DMF (0.8 mL) and DIPEA (0.05 mL, 0.3 mmol) and the reaction mixture was stirred at rt for 2 h. The reaction mixture was concentrated, diluted with EtOAc (˜1.5 mL) and washed with water (1 mL). The organic component was concentrated and the residue was dissolved into MeOH, filtered and purified by preparative HPLC to yield the title compound (25 mg). LC-MS retention time=2.15 min; m/z=843.5 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, methanol-d₄) δ ppm 9.38 (s,1H), 9.36 (s,1H), 8.18 (dd, J=14.5, 8.4 Hz, 2H), 8.07-7.86 (m, 4H), 7.48-7.23 (m, 3H), 6.83-6.74 (m, 2H), 6.59-6.49 (m, 4H), 4.97-4.91 (m, 2H), 3.38 (br. s., 3H), 3.37 (br. s., 3H), 3.20-3.10 (m, 2H), 2.96 (td, J=14.3, 7.9 Hz, 2H).

### Example 127

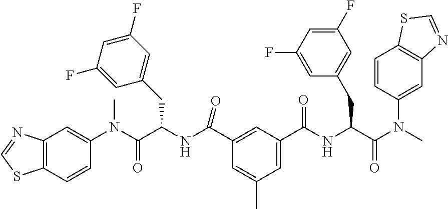 [SMILES: Cc1cc(C(=O)N[C@@H](Cc2cc(F)cc(F)c2)C(=O)N(C)c2ccc3scnc3c2)cc(C(=O)N[C@@H](Cc2cc(F)cc(F)c2)C(=O)N(C)c2ccc3scnc3c2)c1]

HATU (39 mg, 0.10 mmol) was added to a stirred solution of 5-methylisophthalic acid (8.7 mg, 0.048 mmol) and an HCl salt of Intermediate 20 (40.8 mg, 0.106 mmol) in DMF (1 mL) and DIPEA (0.05 mL, 0.3 mmol) and the reaction mixture was stirred at rt for 2 h. The reaction mixture was concentrated, diluted with EtOAc (˜1.5 mL) and washed with water (1 mL). The organic component was concentrated and the residue was dissolved into MeOH, filtered and purified by preparative HPLC to yield the title compound (22 mg). LC-MS retention time=3.29 min; m/z=839.9 [M+H]⁺. (Column: Waters BEH C18, 2.0×50 mm, 1.7-μm particles. Solvent A=95% Water: 5% MeOH: 10 mM NH₄OAc. Solvent B=5% Water: 95% MeOH: 10 mM NH₄OAc. Flow Rate=0.5 mL/min. Start % B=0. Final % B=100. Gradient Time=3.5 minutes. Wavelength=220 nm). ¹H NMR (500 MHZ, methanol-d₄) δ ppm 9.36 (s, 2H), 8.18 (d, J=8.5 Hz, 2H), 8.04-7.89 (m, 3H), 7.69 (s, 2H), 7.43 (br. s., 2H), 6.77 (t, J=9.2 Hz, 2H), 6.53 (d, J=6.1 Hz, 4H), 4.96 (t, J=7.3 Hz, 2H), 3.41 (s, 6H), 3.17 (dd, J=13.4, 6.4 Hz, 2H), 3.05-2.98 (m, 2H), 2.42 (s, 3H).

### Example 128

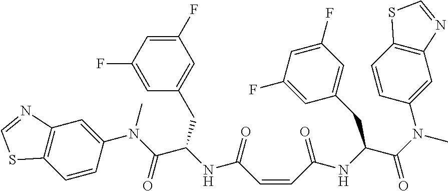 [SMILES: CN(C(=O)[C@H](Cc1cc(F)cc(F)c1)NC(=O)/C=C\C(=O)N[C@@H](Cc1cc(F)cc(F)c1)C(=O)N(C)c1ccc2scnc2c1)c1ccc2scnc2c1]

HATU (39 mg, 0.10 mmol) was added to a stirred solution of maleic acid (5.6 mg, 0.048 mmol) and an HCl salt of Intermediate 20 (40.8 mg, 0.106 mmol) in DMF (1 mL) and DIPEA (0.05 mL, 0.3 mmol) and the reaction mixture was stirred at rt for 2 h. The reaction mixture was concentrated, diluted with EtOAc (˜1.5 mL) and washed with water (1 mL). The organic component was concentrated and the residue was dissolved into MeOH, filtered and purified by preparative HPLC to yield the title compound (14.6 mg). LC-MS retention time=2.18 min; m/z=775.0 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 129

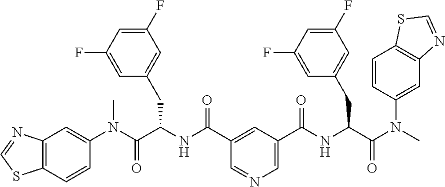 [SMILES: CN(C(=O)[C@H](Cc1cc(F)cc(F)c1)NC(=O)c1cncc(C(=O)N[C@@H](Cc2cc(F)cc(F)c2)C(=O)N(C)c2ccc3scnc3c2)c1)c1ccc2scnc2c1]

HATU (38 mg, 0.10 mmol) was added to a stirred solution of pyridine-3,5-dicarboxylic acid (8.0 mg, 0.048 mmol) and an HCl salt of Intermediate 20 (40.4 mg, 0.105 mmol) in DMF (0.8 mL) and DIPEA (0.05 mL, 0.3 mmol) and the reaction mixture was stirred at rt for 2 h. The reaction mixture was concentrated, diluted with EtOAc (˜1.5 mL) and washed with water (1 mL). The organic component was concentrated and the residue was dissolved into MeOH, filtered and purified by preparative HPLC to yield the title compound (25 mg). LC-MS retention time=2.01 min; m/z=826.2 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, methanol-d₄) δ ppm 9.36 (s, 2H), 8.98 (s, 2H), 8.46 (br. s., 1H), 8.18 (d, J=8.5 Hz, 2H), 8.04-7.90 (m, 2H), 7.42 (br. s., 2H), 6.78 (t, J=8.9 Hz, 2H), 6.53 (d, J=6.7 Hz, 4H), 5.00-4.94 (m, 2H), 3.41 (s, 6H), 3.22-3.15 (m, 2H), 3.04-2.98 (m, 2H).

### Example 131 and Example 134

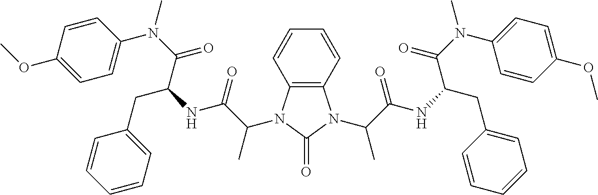

To a solution of an HCl salt of Intermediate 4 (32 mg, 0.10 mmol), Intermediate 66 (13 mg, 0.047 mmol) and DIPEA (0.065 mL, 0.37 mmol) in DMF (0.5 mL) was added HATU (37 mg, 0.098 mmol). The reaction mixture was stirred at rt for 2 h and then purified by preparative HPLC to afford Example 131 (the first eluting peak, absolute stereochemistry unknown) as a white solid (5.4 mg). LC-MS retention time=2.23 min; m/z=811.9 [M+H]⁺. (Column: Waters BEH C18, 2.0×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B, 0-100% B over 3 minutes, then a 0.5-minute hold at 100% B; Flow: 1 mL/min; Detection: UV at 220 nm).

Example 134 (16 mg) was isolated as a mixture of diastereomers (the second and third eluting peaks, inseparable) during the preparative HPLC purification. LC-MS retention time=2.26, 2.28 min; m/z=811.9 [M+H]⁺. (Column: Waters BEH C18, 2.0×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B, 0-100% B over 3 minutes, then a 0.5-minute hold at 100% B; Flow: 1 mL/min; Detection: UV at 220 nm).

### Example 132 and Example 133

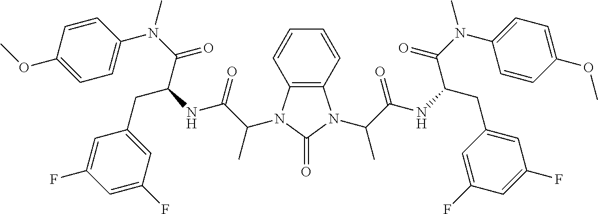

To a solution of an HCl salt of Intermediate 13 (35.0 mg, 0.098 mmol), Intermediate 66 (13 mg, 0.047 mmol) and DIPEA (0.065 mL, 0.37 mmol) in DMF (0.5 mL) was added HATU (37 mg, 0.010 mmol). The reaction mixture was stirred at rt for 2 h and then purified by preparative HPLC to afford Example 132 (the first eluting peak, absolute stereochemistry unknown) as a white solid (7 mg). LC-MS retention time=2.31 min; m/z=883.9 [M+H]⁺. (Column: Waters BEH C18, 2.0×50 mm, 1.7-μm particles; Mobile Phase A: 5:95 acetonitrile:water with 10 mM ammonium acetate; Mobile Phase B: 95:5 acetonitrile: water with 10 mM ammonium acetate; Temperature: 50° C.; Gradient: 0% B, 0-100% B over 3 minutes, then a 0.5-minute hold at 100% B; Flow: 1 mL/min; Detection: UV at 220 nm).

Example 133 (21 mg) was isolated as a mixture of diastereomers (the second and third eluting peaks did not separate) during the preparative HPLC purification. LC-MS retention time=2.36, 2.37 min; m/z=883.9 [M+H]⁺. (Column: Waters BEH C18, 2.0×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B, 0-100% B over 3 minutes, then a 0.5-minute hold at 100% B; Flow: 1 mL/min; Detection: UV at 220 nm).

### Example 135

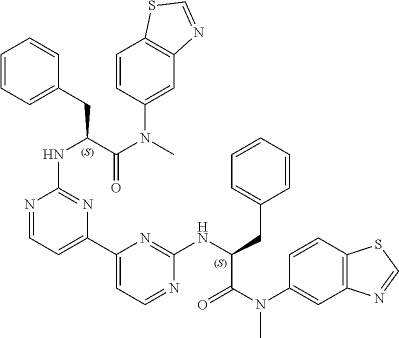

In a 10 mL pressure tube was added Intermediate 18 (50 mg, 0.16 mmol), 2,2′-dichloro-4,4′-bipyrimidine (18.2 mg, 0.080 mmol), BINAP (40.0 mg, 0.064 mmol), cesium carbonate (157 mg, 0.482 mmol), dioxane (2 mL) and the reaction mixture was degasified with nitrogen gas for 5 min. Pd(OAc)₂ (5.8 mg, 0.026 mmol) was added to the above reaction mixture and stirred at 90° C. for 5 h. The reaction mixture was cooled to RT; diluted with water (10 mL) and extracted with EtOAc (2×20 mL). The combined organic layer was washed with water (25 mL), brine (25 mL), dried (Na₂SO₄), filtered and concentrated. The crude product was purified by preparative LC-MS to afford the title product (7 mg) as a pale yellow solid. LC-MS retention time=2.03 min; m/z=777.3 [M+H]⁺. Column: Ascentis Express C18 (50×2.1) mm, 2.7 μm; Flow: 1.1 mL/min; Mobile Phase A: 10 mM NH₄OAc in water: ACN (95:5); Mobile Phase B: 10 mM NH₄OAc in water: ACN (5:95); Temperature: 50° C.; 0% B to 100% B over 3 minutes; UV Detection at 220 nm.

### Example 136

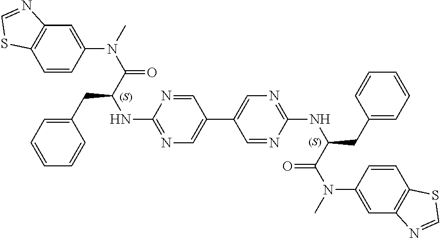

To a stirred solution of Intermediate BB-5 (82 mg, 0.175 mmol) in dioxane (10 mL), water (3 mL) mixture was added Intermediate BB-6 (100 mg, 0.194 mmol), K₃PO₄ (103 mg, 0.485 mmol) and the reaction mixture was degasified with nitrogen for 10 min. PdCl₂(dppf) CH₂Cl₂ adduct (12.7 mg, 0.016 mmol) was added the above reaction mixture and the solution was heated to reflux and stirred for 16 h. The reaction mixture was cooled RT, diluted with saturated NH₄Cl solution (50 mL) and extracted with EtOAc (2×50 mL). The combined organic layer was washed with brine (75 mL), dried (Na₂SO₄), filtered and concentrated. The crude material was purified by preparative HPLC to afford the title product (32 mg) as red liquid. LC-MS retention time=1.99 min; m/z=777.3 [M+H]⁺. Column: Ascentis Express C18 (50×2.1) mm, 2.7 μm; Flow: 1.1 mL/min; Mobile Phase A: 10 mM NH₄OAc in water: ACN (95:5); Mobile Phase B: 10 mM NH₄OAc in water: ACN (5:95); Temperature: 50° C.; 0% B to 100% B over 3 minutes; UV Detection at 220 nm. ¹H NMR (400 MHz, DMSO-d₆) δ 9.50 (s, 2H), 8.47 (br. s., 4H), 8.29 (d, J=8.0 Hz, 2H), 8.16 (br. s., 2H), 7.59 (d, J=6.5 Hz, 2H), 7.52 (d, J=8.0 Hz, 2H), 7.07 (br. s., 6H), 6.81 (br. s., 4H), 4.62 (br. s., 2H), 3.25 (s, 6H), 3.00-2.90 (m, 2H), 2.90-2.83 (m, 2H).

### Example 138

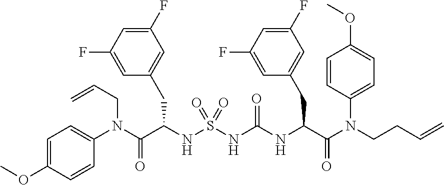 [SMILES: C=CCCN(C(=O)[C@H](Cc1cc(F)cc(F)c1)NC(=O)NS(=O)(=O)N[C@@H](Cc1cc(F)cc(F)c1)C(=O)N(CC=C)c1ccc(OC)cc1)c1ccc(OC)cc1]

A solution of an HCl salt of Intermediate 32 (45 mg, 0.10 mmol) in DCM (0.7 mL) was added dropwise to a vigorously stirred solution of sulfurisocyanatidic chloride (19 mg, 0.13 mmol) in DCM (0.5 mL) and the reaction mixture was stirred at rt for 30 min. The reaction mixture was then treated with a solution of TEA (0.09 mL, 0.6 mmol) and an HCl salt of Intermediate 33 (47 mg, 0.10 mmol) in DCM (0.5 mL) and stirred at rt for 3 h. The crude reaction mixture was concentrated and the residue was dissolved into MeOH, filtered and purified by preparative HPLC to yield the title compound (44.7 mg). LC-MS retention time=2.25 min; m/z=812.8 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, methanol-d₄) δ ppm 7.43-7.03 (m, 2H), 6.98 (d, J=6.7 Hz, 4H), 6.82-6.74 (m, 2H), 6.54 (d, J=7.0 Hz, 2H), 6.46 (d, J=6.4 Hz, 2H), 5.81-5.69 (m, 2H), 5.09-5.05 (m, 2H), 5.04-4.98 (m, 2H), 4.59-4.56 (m, 1H), 4.28-4.15 (m, 3H), 3.86 (s, 3H), 3.82 (s, 3H), 3.76 (dt, J=13.7, 7.2 Hz, 1H), 3.69-3.61 (m, 1H), 2.76-2.67 (m, 2H), 2.24 (q, J=7.0 Hz, 2H).

### Example 139

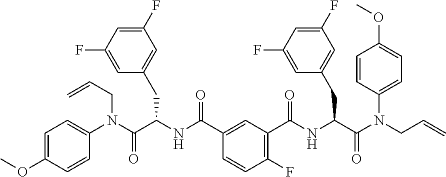 [SMILES: C=CCN(C(=O)[C@H](Cc1cc(F)cc(F)c1)NC(=O)c1ccc(F)c(C(=O)N[C@@H](Cc2cc(F)cc(F)c2)C(=O)N(CC=C)c2ccc(OC)cc2)c1)c1ccc(OC)cc1]

HATU (66 mg, 0.17 mmol) was added to a stirred solution of an HCl salt of Intermediate 32 (60 mg, 0.16 mmol) and 4-fluoroisophthalic acid (14 mg, 0.078 mmol) in DMF (1 mL) and DIPEA (0.07 mL, 0.4 mmol) and the reaction mixture was stirred at rt for 3 h. The crude reaction mixture was concentrated and the residue was dissolved into MeOH, filtered and purified by preparative HPLC to yield the title compound (51.2 mg). LC-MS retention time=2.45 min; m/z=841.8 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm). ¹H NMR (500 MHZ, methanol-d₄) δ ppm 8.08 (d, J=4.9 Hz, 1H), 7.96-7.90 (m, 1H), 7.32-6.93 (m, 9H), 6.85-6.76 (m, 2H), 6.56 (dd, J=13.9, 6.6 Hz, 4H), 5.91-5.79 (m, 2H), 5.18-5.07 (m, 4H), 4.64-4.59 (m, 2H), 4.37-4.30 (m, 2H), 4.28-4.21 (m, 2H), 3.87 (s, 6H), 3.15-3.05 (m, 2H), 2.93 (ddd, J=18.3, 13.6, 8.7 Hz, 2H).

### Example 140

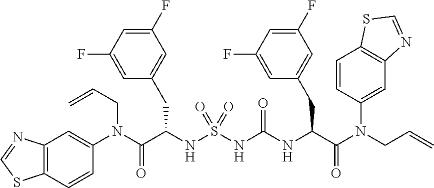 [SMILES: C=CCN(C(=O)[C@H](Cc1cc(F)cc(F)c1)NC(=O)NS(=O)(=O)N[C@@H](Cc1cc(F)cc(F)c1)C(=O)N(CC=C)c1ccc2scnc2c1)c1ccc2scnc2c1]

A solution of sulfurisocyanatidic chloride (20 mg, 0.14 mmol) in DCM (0.30 mL) was added dropwise to a solution of an HCl salt of Intermediate 38 (112 mg, 0.233 mmol) in DCM (1.5 mL) and TEA (0.130 mL, 0.933 mmol) stirred at 0° C. The reaction mixture was allowed to warm to rt and was stirred for 2 h. The crude reaction mixture was concentrated and the residue was dissolved into MeOH, filtered and purified by preparative HPLC to yield the title compound (23.4 mg). LC-MS retention time=2.04 min; m/z=852.7 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 141-142

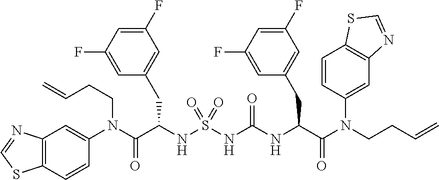 [SMILES: C=CCCN(C(=O)[C@H](Cc1cc(F)cc(F)c1)NC(=O)NS(=O)(=O)N[C@@H](Cc1cc(F)cc(F)c1)C(=O)N(CCC=C)c1ccc2scnc2c1)c1ccc2scnc2c1]

A solution of sulfurisocyanatidic chloride (19.5 mg, 0.137 mmol) in DCM (0.20 mL) was added dropwise to a solution of an HCl salt of Intermediate 39 (111 mg, 0.229 mmol) in DCM (1.5 mL) and TEA (0.13 mL, 0.92 mmol) stirred at 0° C. The reaction mixture was allowed to warm to rt and was stirred 2 h. The crude reaction mixture was concentrated and the residue was dissolved into MeOH, filtered and purified by preparative HPLC to yield two stereoisomers.

Example 141 (second elute; exact stereochemical composition unknown; 29.5 mg). LC-MS retention time=2.13 min; m/z=880.8 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

Example 142 (first elute; exact stereochemical composition unknown; 9.9 mg). LC-MS retention time=2.10 min; m/z=880.7 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 145

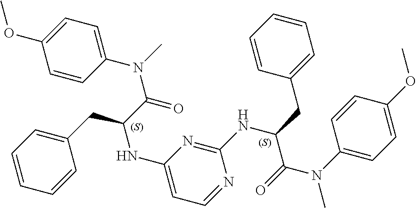

To a stirred solution of Intermediate 4 (107 mg, 0.378 mmol) in dioxane (10 mL) was added Intermediate BB-7 (150 mg, 0.378 mmol), BINAP (35.3 mg, 0.057 mmol), Cs₂CO₃ (308 mg, 0.945 mmol) and the reaction mixture was degasified with nitrogen for 10 min. Pd(OAc)₂ (6.8 mg, 0.030 mmol) was added and the resulting reaction mixture stirred at 90° C. for 3 h. The reaction mixture was diluted with saturated NH₄Cl solution (50 mL) and extracted with EtOAc (2×50 mL). The combined organic layer was washed with brine (75 mL), dried (Na₂SO₄) filtered, concentrated and the crude product was purified by preparative LC/MS to afford the title product as an off white solid (137 mg). LC-MS retention time=2.06 min; m/z=645.4 [M+H]⁺. Column: Ascentis Express C18 (50×2.1) mm, 2.7 μm; Flow: 1.1 mL/min; Mobile Phase A: 10 mM NH₄OAc in water: ACN (95:5); Mobile Phase B: 10 mM NH₄OAc in water: ACN (5:95); Temperature: 50° C.; 0% B to 100% B over 3 minutes; UV Detection at 220 nm. ¹H NMR (400 MHz, at 80° C., DMSO-d₆) δ 7.60 (d, J=5.77 Hz, 1H), 7.13-7.25 (m, 6H), 6.99-7.08 (m, 4H), 6.77-6.99 (m, 9H), 5.81 (d, J=4.77 Hz, 1H), 5.39 (br. s., 1H), 4.74 (br. s., 1H), 3.74 (s, 6H), 3.11 (s, 6H), 2.82-2.99 (m, 2H), 2.64-2.81 (m, 2H).

### Example 146

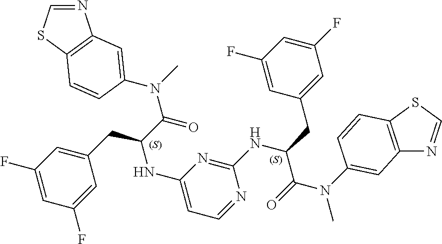

To a stirred solution of Intermediate 20 (100 mg, 0.288 mmol) in dioxane (15 mL) was added Intermediate BB-8 (66.2 mg, 0.144 mmol), BINAP (26.9 mg, 0.043 mmol), Cs₂CO₃ (234 mg, 0.720 mmol) and the reaction mixture was degasified with nitrogen for 10 min. Pd(OAc)₂ (5.17 mg, 0.023 mmol) was added to the above reaction mixture and stirred at 100° C. for 5 h. The reaction mixture was diluted with saturated NH₄Cl solution (50 mL) and extracted with EtOAc (2×50 mL). The combined organic layer was washed with brine (75 mL), dried (Na₂SO₄), filtered and concentrated. The crude product was purified by preparative HPLC to afford the title product (18 mg) as an off white solid. LC-MS retention time=2.04 min; m/z=771.2 [M+H]⁺. Column: Ascentis Express C18 (50×2.1) mm, 2.7 μm; Flow: 1.1 mL/min; Mobile Phase A: 10 mM NH₄OAc in water: ACN (95:5); Mobile Phase B: 10 mM NH₄OAc in water: ACN (5:95); Temperature: 50° C.; 0% B to 100% B over 3 minutes; UV Detection at 220 nm. ¹H NMR (400 MHz, DMSO-d6, 80° C.) δ 9.38 (d, J=10.0 Hz, 2H), 8.14 (t, J=9.2 Hz, 2H), 8.06 (s, 1H), 7.99 (s, 1H), 7.57 (d, J=5.6 Hz, 1H), 7.38-7.32 (m, 2H), 7.01 (br. s., 1H), 6.85 (br. s., 2H), 6.53 (br. s., 4H), 5.83-5.82 (m, 2H), 4.82 (br. s., 2H), 3.27 (s, 6H), 3.00-2.90 (m, 2H), 2.85-2.80 (m, 2H).

### Example 147

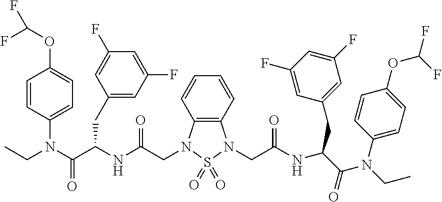 [SMILES: CCN(C(=O)[C@H](Cc1cc(F)cc(F)c1)NC(=O)CN1c2ccccc2N(CC(=O)N[C@@H](Cc2cc(F)cc(F)c2)C(=O)N(CC)c2ccc(OC(F)F)cc2)S1(=O)=O)c1ccc(OC(F)F)cc1]

To a solution of an HCl salt of Intermediate 68 (60 mg, 0.15 mmol), Intermediate 51 (20 mg, 0.070 mmol) and DIPEA (0.10 mL, 0.56 mmol) in DMF (0.8 mL) was added HATU (56 mg, 0.15 mmol). The reaction mixture was stirred at rt for 2 h and then purified by preparative HPLC to afford the title compound as a white solid (52 mg). LC-MS retention time=1.46 min; m/z=991.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 148

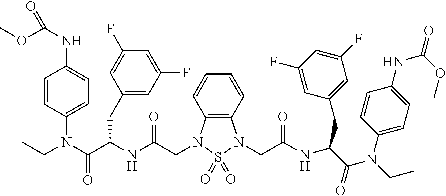 [SMILES: CCN(C(=O)[C@H](Cc1cc(F)cc(F)c1)NC(=O)CN1c2ccccc2N(CC(=O)N[C@@H](Cc2cc(F)cc(F)c2)C(=O)N(CC)c2ccc(NC(=O)OC)cc2)S1(=O)=O)c1ccc(NC(=O)OC)cc1]

To a solution of an HCl salt of Intermediate 69 (60.7 mg, 0.147 mmol), Intermediate 51 (20 mg, 0.070 mmol) and DIPEA (0.10 mL, 0.56 mmol) in DMF (0.5 mL) was added HATU (56 mg, 0.15 mmol). The reaction mixture was stirred at rt for 2 h and then purified by preparative HPLC to afford the title compound as a white solid (52 mg). LC-MS retention time=2.08 min; m/z=1005.1 [M+H]⁺. (Column: Waters BEH C18, 2.0×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B, 0-100% B over 3 minutes, then a 0.5-minute hold at 100% B; Flow: 1 mL/min; Detection: UV at 220 nm).

### Example 149

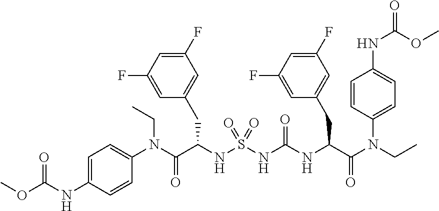

To a slurry of an HCl salt of Intermediate 69 (40 mg, 0.10 mmol) in DCM (1 mL) was added TEA (0.04 mL, 0.3 mmol). The reaction mixture was treated with sulfurisocyanatidic chloride (4.2 μ1, 0.048 mmol) dropwise. The reaction mixture was stirred at rt overnight and then concentrated. The residue was taken up into DMF (1 mL) and then purified by preparative HPLC to afford the title compound as a white solid (17 mg). LC-MS retention time=1.24 min; m/z=860.1 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 150

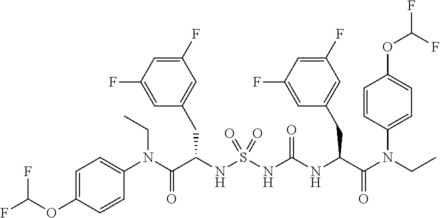 [SMILES: CCN(C(=O)[C@H](Cc1cc(F)cc(F)c1)NC(=O)NS(=O)(=O)N[C@@H](Cc1cc(F)cc(F)c1)C(=O)N(CC)c1ccc(OC(F)F)cc1)c1ccc(OC(F)F)cc1]

To a slurry of an HCl salt of Intermediate 68 (40 mg, 0.10 mmol) in DCM (1 mL) was added TEA (0.04 mL, 0.3 mmol). The reaction mixture was treated with sulfurisocyanatidic chloride (4.2 μl, 0.048 mmol) dropwise. The reaction mixture was stirred at rt overnight, concentrated and then purified by preparative HPLC to afford the title compound as a white solid (21 mg). LC-MS retention time=1.42 min; m/z=846.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 151

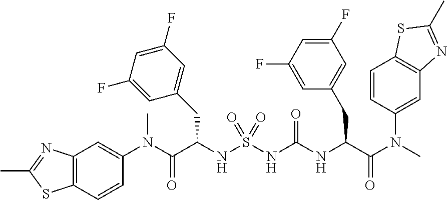

To a solution of sulfurisocyanatidic chloride (0.015 mL, 0.17 mmol) in DCM (0.5 mL) was added a solution of an HCl salt of Intermediate 73 (61 mg, 0.14 mmol) in DCM (1 mL) and TEA (0.020 mL, 0.14 mmol) and the reaction mixture was stirred at rt for 20 min. Then, a solution of 2,3-dihydro-1H-pyrrolo[2,3-b]pyridine (25.3 mg, 0.21 mmol) in DCM (0.5 mL) and TEA (0.078 mL, 0.56 mmol) was added to the reaction mixture, and it was stirred at rt for 4 h. Then reaction mixture was concentrated, the residue was dissolved in MeOH and then purified twice via preparative HPLC to yield the title compound (4.4 mg) as a by-product from the reaction. LC-MS retention time=1.90 min; m/z=828.3 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=1.0 mL/min. Start % B=0. Final % B=100. Gradient Time=3 minutes, then a 0.5-minute hold at 100% B. Wavelength=220).

### Example 152

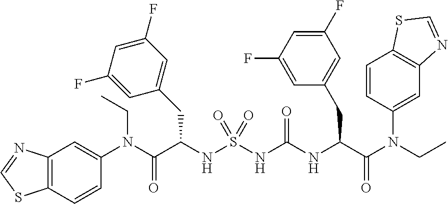 [SMILES: CCN(C(=O)[C@H](Cc1cc(F)cc(F)c1)NC(=O)NS(=O)(=O)N[C@@H](Cc1cc(F)cc(F)c1)C(=O)N(CC)c1ccc2scnc2c1)c1ccc2scnc2c1]

To a solution of sulfurisocyanatidic chloride (0.016 mL, 0.18 mmol) in DCM (1 mL) was added a solution of an HCl salt of Intermediate 78 (66 mg, 0.15 mmol) in DCM (1 mL) and TEA (0.021 mL, 0.15 mmol) and the reaction mixture was stirred for 30 min. Then a solution of 2,3-dihydro-1H-pyrrolo[2,3-b]pyridine (27.4 mg, 0.22 mmol) in DCM (1 mL) and TEA (0.085 mL, 0.61 mmol) was added to the reaction mixture and it was stirred at rt for 4 h. The reaction mixture was concentrated, the residue was dissolved in MeOH and then purified via preparative LC/MS (Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-85% B over 40 minutes, then a 5-minute hold at 100% B; Flow: 20 mL/min. Fractions containing the title compound were combined and dried via centrifugal evaporation.) to yield the title compound (8.3 mg) as a by-product from the reaction. LC-MS retention time=2.02 min; m/z=827.9 [M+H]⁺. (Column: Waters BEH C18, 2.0×50 mm, 1.7-μm particles. Solvent A=95% Water: 5% Acetonitrile: 10 mM NH₄OAc. Solvent B=5% Water: 95% Acetonitrile: 10 mM NH₄OAc. Flow Rate=1.0 mL/min. Start % B=0. Final % B=100. Gradient Time=3 minutes, then a 0.5-minute hold at 100% B. Wavelength=220).

### Example 153

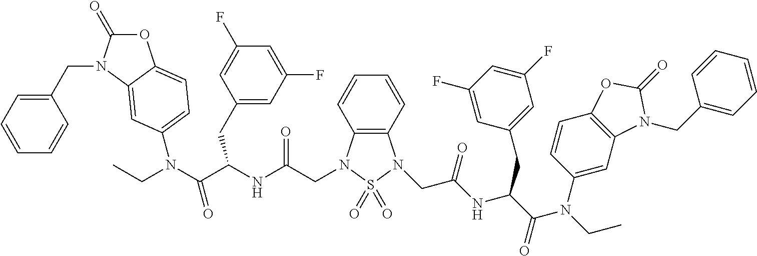 [SMILES: CCN(C(=O)[C@H](Cc1cc(F)cc(F)c1)NC(=O)CN1c2ccccc2N(CC(=O)N[C@@H](Cc2cc(F)cc(F)c2)C(=O)N(CC)c2ccc3oc(=O)n(Cc4ccccc4)c3c2)S1(=O)=O)c1ccc2oc(=O)n(Cc3ccccc3)c2c1]

To a solution of an HCl salt of Intermediate 70 (61 mg, 0.15 mmol), Intermediate 51 (20 mg, 0.070 mmol) and DIPEA (0.10 mL, 0.56 mmol) in DMF (0.5 mL) was added HATU (56 mg, 0.15 mmol). The reaction mixture was stirred at rt for 2 h and then purified by preparative HPLC to afford the title compound as a white solid (22 mg). LC-MS retention time=1.49 min; m/z=1153.2 [M+H]⁺. (Column: Waters Aquity BEH C18 2.1×50 mm 1.7-μm-particles; Solvent A=100% Water/0.05% TFA; Solvent B=100% Acetonitrile/0.05% TFA; Flow Rate=0.8 mL/min. Start % B=2; Final % B=98; Gradient Time=1.5 minutes; Wavelength=220 nm).

### Example 154

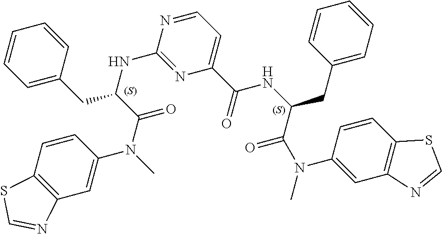

To a stirred solution of Intermediate BB-9 (100 mg, 0.166 mmol) in dioxane (5 mL) was added Intermediate 18 (51.7 mg, 0.166 mmol), cesium carbonate (162 mg, 0.498 mmol) and reaction mixture was purged with nitrogen for 10 min. Then added BINAP (20.7 mg, 0.033 mmol), Pd(OAc)₂ (3.73 mg, 0.017 mmol) and the reaction mixture was heated to reflux and stirred for 16 h. The reaction mixture was cooled to RT, diluted with saturated NH₄Cl solution (50 mL) and extracted with EtOAc (2×50 mL). The combined organic layer was washed with brine (75 mL), dried (Na₂SO₄), filtered and concentrated. The crude product was purified by preparative HPLC to afford the title product (19.5 mg) as light brown solid. LC-MS retention time=2.82 min; m/z=727.2 [M+H]⁺. Column: KINETIX XB-C18, 75×3 mm, 2.6 μm; Flow rate: 1 mL/min; Mobile Phase A: 10 mM HCOONH₄ in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm.

### Example 155

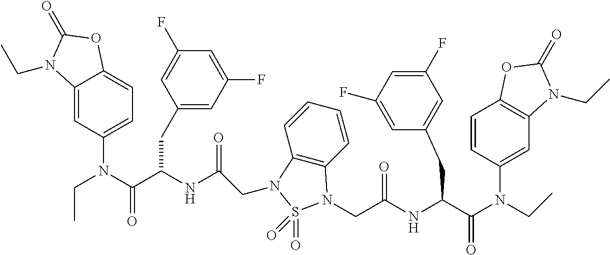 [SMILES: CCN(C(=O)[C@H](Cc1cc(F)cc(F)c1)NC(=O)CN1c2ccccc2N(CC(=O)N[C@@H](Cc2cc(F)cc(F)c2)C(=O)N(CC)c2ccc3oc(=O)n(CC)c3c2)S1(=O)=O)c1ccc2oc(=O)n(CC)c2c1]

To a solution of an HCl salt of Intermediate 80 (46.9 mg, 0.110 mmol), Intermediate 51 (15 mg, 0.052 mmol) and DIPEA (0.073 mL, 0.42 mmol) in DMF (0.5 mL) was added HATU (41.8 mg, 0.110 mmol) and the reaction mixture was stirred at rt for 3 h and then purified by preparative HPLC to afford the title compound (20 mg) as a white solid. LC-MS retention time=1.36 min; m/z=1029.50 [M+H]+ (Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7 μm, Oven Temp.=40° C.).

### Example 156

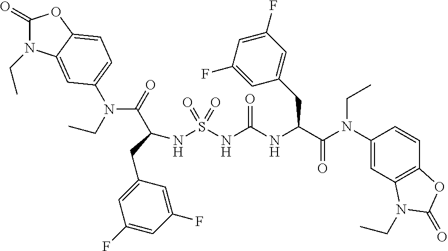 [SMILES: CCN(C(=O)[C@H](Cc1cc(F)cc(F)c1)NC(=O)NS(=O)(=O)N[C@@H](Cc1cc(F)cc(F)c1)C(=O)N(CC)c1ccc2oc(=O)n(CC)c2c1)c1ccc2oc(=O)n(CC)c2c1]

To a slurry of an HCl salt of Intermediate 80 (45 mg, 0.11 mmol) in DCM (1 mL) was added TEA (0.044 mL, 0.32 mmol). To the resulting solution was added sulfurisocyanatidic chloride (4.6 μL, 0.053 mmol) dropwise. The final solution was stirred at rt overnight. The solvent was removed in vacuo and the residue was taken up into DMF (1 mL), filtered, and purified by preparative HPLC to yield the title compound (6 mg) as a white solid. LC-MS retention time=1.33 min; m/z=884.45 [M+H]+ (Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7 μm, Oven Temp.=40° C.).

### Example 158

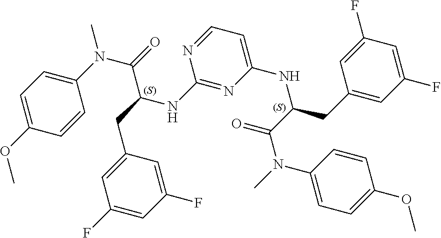

To a stirred solution of Intermediate 13 (148 mg, 0.462 mmol) in dioxane (10 mL) was added Intermediate BB-10 (200 mg, 0.462 mmol), BINAP (43.2 mg, 0.069 mmol), Cs₂CO₃ (376 mg, 1.16 mmol) and the reaction mixture was degasified with nitrogen for 10 min. Pd(OAc)₂ (8.30 mg, 0.037 mmol) was added and the resulting reaction mixture was stirred at 90° C. for 3 h. The reaction mixture was diluted with saturated NH₄Cl solution (50 mL) and extracted with EtOAc (2×50 mL). The combined organic layer was washed with brine (75 mL), dried (Na₂SO₄), filtered, concentrated and the crude material was purified by preparative HPLC to afford the title product (137 mg) as an off white solid. LC-MS retention time=2.2 min; m/z=717.4 [M+H]⁺. Column: Ascentis Express C18 (50×2.1) mm, 2.7 μm; Flow: 1.1 mL/min; Mobile Phase A: 10 mM NH₄OAc in water: ACN (95:5); Mobile Phase B: 10 mM NH₄OAc in water: ACN (5:95); Temperature: 50° C.; 0% B to 100% B over 3 minutes; UV Detection at 220 nm. ¹H NMR (400 MHz, 80° C., DMSO-d₆) δ 7.61 (d, J=5.77 Hz, 1H), 7.20 (d, J=7.03 Hz, 4H), 7.05-6.82 (m, 7H), 6.57 (br. s., 4H), 5.82 (d, J=5.27 Hz, 1H), 5.63 (br. s., 1H), 4.75 (br. s., 2H), 3.77 (s, 6H), 3.15 (s, 6H), 2.99-2.85 (m, 2H), 2.84-2.72 (m, 2H).

### Example 159

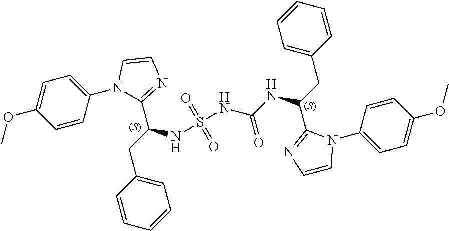

To a stirred solution of Intermediate BB-14 (124 mg, 0.424 mmol) in DCM (10 mL) was added sulfurisocyanatidic chloride (0.012 mL, 0.14 mmol), TEA (0.118 mL, 0.848 mmol) at 0° C. and the reaction mixture was stirred for 30 min at 0° C. and at room temperature for 2 h. The reaction mixture was concentrated to dryness and the crude product was purified by preparative HPLC to afford the title product (1.5 mg) as an off white solid. LC-MS retention time=1.405 min; m/z=692.3 [M+H]⁺. Column: Ascentis Express C18 (50×2.1) mm, 2.7 μm; Flow: 1.1 mL/min; Mobile Phase A: 10 mM NH₄OAc in water: ACN (95:5); Mobile Phase B: 10 mM NH₄OAc in water: ACN (5:95); Temperature: 50° C.; 0% B to 100% B over 3 minutes; UV Detection at 220 nm.

### Example 160

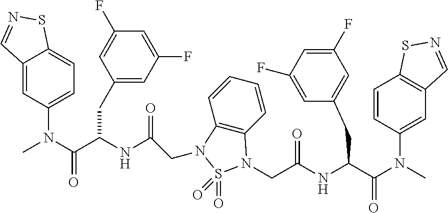 [SMILES: CN(C(=O)[C@H](Cc1cc(F)cc(F)c1)NC(=O)CN1c2ccccc2N(CC(=O)N[C@@H](Cc2cc(F)cc(F)c2)C(=O)N(C)c2ccc3sncc3c2)S1(=O)=O)c1ccc2sncc2c1]

To a solution of an HCl salt of Intermediate 81 (30.8 mg, 0.073 mmol), Intermediate 51 (10 mg, 0.035 mmol) and DIPEA (0.049 mL, 0.28 mmol) in DMF (0.5 mL) was added HATU (28 mg, 0.073 mmol) and the reaction mixture was stirred at rt for 3 h. The reaction mixture was then filtered and purified by preparative HPLC to yield the title compound (8.7 mg) as a white solid. LC-MS retention time=1.36 min; m/z=944.95 [M+H]+ (Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7 μm, Oven Temp.=40° C.).

### Example 161

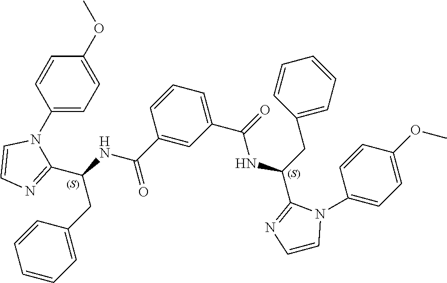

To a stirred solution of Intermediate BB-14 (106 mg, 0.361 mmol) and isophthalic acid (20 mg, 0.120 mmol) in DMF (8 mL) was added DIPEA (0.13 mL, 0.72 mmol) and HATU (137 mg, 0.361 mmol) and the reaction mixture was stirred at room temperature for 16 h. The reaction mixture was concentrated to dryness and the crude product was purified by preparative HPLC to afford the title product (36 mg) as an off white solid. LC-MS retention time=2.18 min; m/z=717.4 [M+H]⁺. Column: Ascentis Express C18 (50×2.1) mm, 2.7 pin; Flow: 1.1 mL/min; Mobile Phase A: 10 mM NH₄OAc in water: ACN (95:5); Mobile Phase B: 10 mM NH₄OAc in water: ACN (5:95); Temperature: 50° C.; 0% B to 100% B over 3 minutes; UV Detection at 220 nm. ¹H NMR (400 MHz, DMSO-d₆) δ 9.07-9.02 (m, 2H), 8.21 (s, 1H), 7.91 (dd, J=7.5, 1.5 Hz, 2H), 7.50 (t, J=7.8 Hz, 1H), 7.18-7.12 (m, 8H), 7.08-7.01 (m, 6H), 6.98-6.92 (m, 8H), 5.21-5.14 (m, 2H), 3.79 (s, 6H), 3.22 (dd, J=13.2, 8.0 Hz, 1H), 3.14 (dd, J=12.8, 7.2 Hz, 1H).

### Example 162

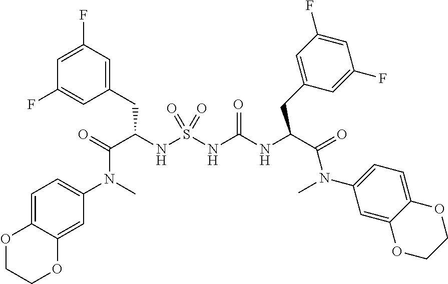 [SMILES: CN(C(=O)[C@H](Cc1cc(F)cc(F)c1)NC(=O)NS(=O)(=O)N[C@@H](Cc1cc(F)cc(F)c1)C(=O)N(C)c1ccc2c(c1)OCCO2)c1ccc2c(c1)OCCO2]

To a solution of sulfurisocyanatidic chloride (0.012 mL, 0.14 mmol) in DCM (0.5 mL) was added a DCM (1 mL) solution of an HCl salt of Intermediate 171 (48 mg, 0.12 mmol) and TEA (0.017 mL, 0.12 mmol) and the reaction mixture was stirred at rt for 20 min. Then a solution of 2,3-dihydro-1H-pyrrolo[2,3-b]pyridine (22.5 mg, 0.18 mmol) in DCM (0.5 mL) and TEA (0.070 mL, 0.5 mmol) was added and the reaction mixture was stirred at rt for 16 h. The reaction was filtered, and purified by preparative HPLC to afford the title compound (10.1 mg). LC-MS retention time=2.33 min; m/z=802.0 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Example 163

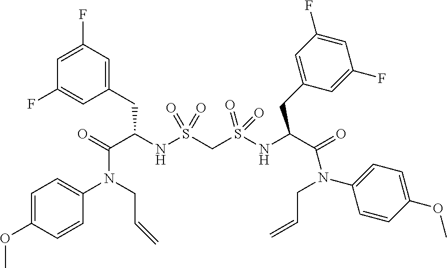 [SMILES: C=CCN(C(=O)[C@H](Cc1cc(F)cc(F)c1)NS(=O)(=O)CS(=O)(=O)N[C@@H](Cc1cc(F)cc(F)c1)C(=O)N(CC=C)c1ccc(OC)cc1)c1ccc(OC)cc1]

An HCl salt of Intermediate 32 (41 mg, 0.107 mmol) and methanedisulfonyl dichloride (19 mg, 0.089 mmol) were dissolved into DCM (1 mL) and then treated with TEA (0.075 mL, 0.54 mmol) and the reaction mixture was stirred at rt ON. The reaction was filtered, and purified by preparative HPLC to afford the title compound (19.6 mg). LC-MS retention time=2.50 min; m/z=832.9 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Example 164

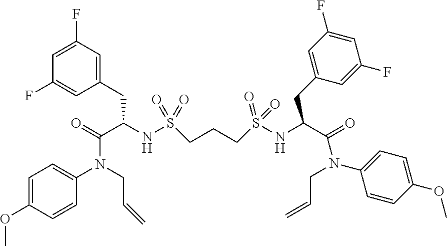 [SMILES: C=CCN(C(=O)[C@H](Cc1cc(F)cc(F)c1)NS(=O)(=O)CCCS(=O)(=O)N[C@@H](Cc1cc(F)cc(F)c1)C(=O)N(CC=C)c1ccc(OC)cc1)c1ccc(OC)cc1]

An HCl salt of Intermediate 32 (41 mg, 0.11 mmol) and propane-1,3-disulfonyl dichloride (25.8 mg, 0.107 mmol) were dissolved into DCM (1 mL) and then treated with TEA (0.075 mL, 0.56 mmol) and the reaction mixture was stirred at rt ON. The reaction was filtered, and purified by preparative HPLC to afford the title compound (9.6 mg). LC-MS retention time=2.46 min; m/z=860.9 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Example 165

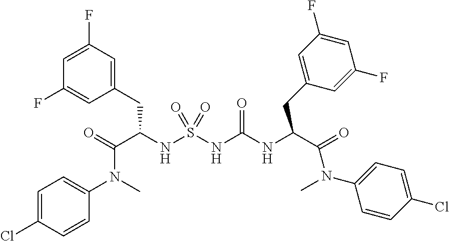 [SMILES: CN(C(=O)[C@H](Cc1cc(F)cc(F)c1)NC(=O)NS(=O)(=O)N[C@@H](Cc1cc(F)cc(F)c1)C(=O)N(C)c1ccc(Cl)cc1)c1ccc(Cl)cc1]

To a solution of sulfurisocyanatidic chloride (0.012 mL, 0.14 mmol) in DCM (0.5 mL) was added a DCM (1 mL) solution of an HCl salt of Intermediate 173 (46.4 mg, 0.13 mmol) and the mixture was stirred for 20 min. Then a solution of 2,3-dihydro-1H-pyrrolo[2,3-b]pyridine (23.1 mg, 0.19 mmol) in DCM (0.5 mL) and TEA (0.072 mL, 0.51 mmol) was added to the reaction mixture and it was stirred at rt for 16 h. The reaction was filtered, and purified by preparative HPLC to afford the title compound (4.8 mg). LC-MS retention time=2.66 min; m/z=753.8 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Example 166

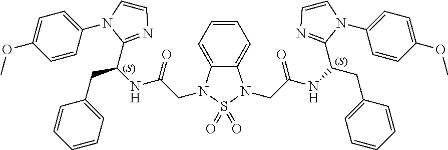

To a stirred solution of Intermediate 51 dihydrochloride (13 mg, 0.04 mmol), DIPEA (0.051 mL, 0.29 mmol) and HATU (41.5 mg, 0.11 mmol) in DMF (3 mL) was added Intermediate BB-14 (30 mg, 0.09 mmol) and stirred the reaction mixture for 16 h. The reaction mixture quenched into water (30 mL) and extracted with EtOAc (3×20 mL). The combined organic layer was washed with water (20 mL), brine (20 mL), dried (Na₂SO₄), filtered and concentrated. The crude product was purified by preparative HPLC to afford the title compound (8 mg) as an off white solid. LC-MS retention time=2.45 min; m/z=837.3 [M+H]⁺. Column: Ascentis Express C18 (50×2.1) mm, 2.7 μm; Flow: 1.1 mL/min; Mobile Phase A: 10 mM NH₄OAc in water: ACN (95:5); Mobile Phase B: 10 mM NH₄OAc in water: ACN (5:95); Temperature: 50° C.; 0% B to 100% B over 3 minutes; UV Detection at 220 nm. ¹H NMR (400 MHz, DMSO-d₆) δ 8.97 (d, J=8.0 Hz, 2H), 7.18-7.15 (m, 6H), 7.13 (d, J=1.0 Hz, 2H), 7.08 (d, J=1.0 Hz, 2H), 6.90-6.84 (m, 14H), 6.57-6.54 (m, 2H), 4.97-4.90 (dd, J=14.8, 8.0 Hz, 2H), 4.32 (dd, J=16.8 Hz, 4H), 3.75 (s, 6H), 3.15 (dd, J=13.1, 8.5 Hz, 2H), 3.00 (dd, J=13.1, 6.5 Hz, 2H).

### Example 167

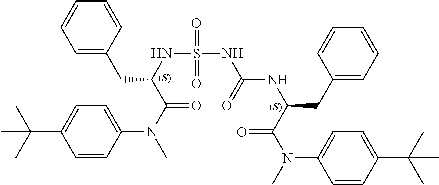

To a stirred solution of Intermediate BB-16 (65.8 mg, 0.212 mmol) in DCM (5 mL) was added DIPEA (0.056 mL, 0.318 mmol), sulfurisocyanatidic chloride (15 mg, 0.106 mmol) at 0° C. and the reaction mixture was warmed to room temperature and stirred for 1 h. The reaction mixture was concentrated to dryness; the crude material was purified via preparative HPLC to afford the title compound as an off white solid (2.7 mg). LC-MS retention time=2.62 min; m/z=726.5 [M+H]⁺. Column: Ascentis Express C18 (50×2.1) mm, 2.7 μm; Flow: 1.1 mL/min; Mobile Phase A: 10 mM NH₄OAc in water: ACN (95:5); Mobile Phase B: 10 mM NH₄OAc in water: ACN (5:95); Temperature: 50° C.; 0% B to 100% B over 3 minutes; UV Detection at 220 nm.

### Example 168

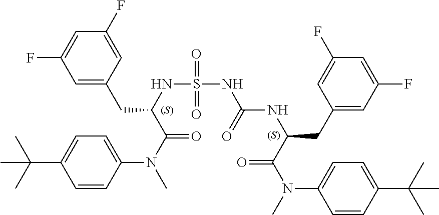

To a stirred solution of Intermediate BB-18 (50 mg, 0.14 mmol) in DCM (10 mL) was added chlorosulfonyl isocyanate (30.6 mg, 0.217 mmol), TEA (1.0 eq.) at 0° C. and the reaction mixture was stirred for 30 min. Then added 2,3-dihydro-1H-pyrrolo[2,3-b]pyridine (26.0 mg, 0.217 mmol) in DCM (10 mL) followed by TEA (0.101 mL, 0.722 mmol) at 0° C. and the reaction mixture was stirred at room temperature for 2 h. The reaction mixture was concentrated to dryness and the crude product was purified by preparative HPLC to afford the title compound (6.5 mg, an off white solid). Title compound: LC-MS retention time=2.93 min; m/z=798.3 [M+H]⁺. Column: Ascentis Express C18 (50×2.1) mm, 2.7 μm; Flow: 1.1 mL/min; Mobile Phase A: 10 mM NH₄OAc in water: ACN (95:5); Mobile Phase B: 10 mM NH₄OAc in water: ACN (5:95); Temperature: 50° C.; 0% B to 100% B over 3 minutes; UV Detection at 220 nm.

### Example 169

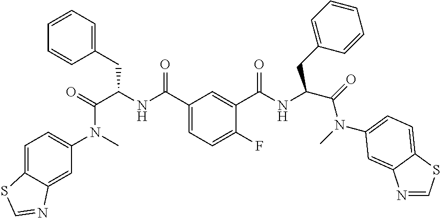 [SMILES: CN(C(=O)[C@H](Cc1ccccc1)NC(=O)c1ccc(F)c(C(=O)N[C@@H](Cc2ccccc2)C(=O)N(C)c2ccc3scnc3c2)c1)c1ccc2scnc2c1]

To a mixture of an HCl salt of Intermediate 18 (50 mg, 0.13 mmol) and 4-fluoroisophthalic acid (12 mg, 0.065 mmol) in DMF and DIPEA (68 μl, 0.39 mmol), HATU (51.9 mg, 0.14 mmol) was added and the reaction mixture was stirred at rt for 3 h. The reaction was filtered, and purified by preparative HPLC to afford the title compound (27.3 mg). LC-MS retention time=2.04 min; m/z=770.9 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Example 170

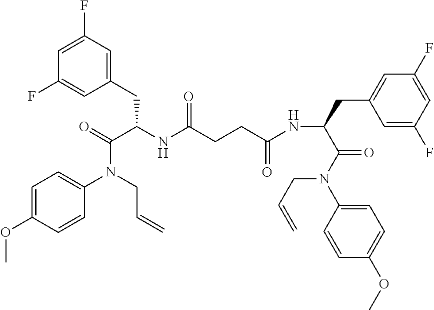 [SMILES: C=CCN(C(=O)[C@H](Cc1cc(F)cc(F)c1)NC(=O)CCC(=O)N[C@@H](Cc1cc(F)cc(F)c1)C(=O)N(CC=C)c1ccc(OC)cc1)c1ccc(OC)cc1]

TEA (0.12 mL, 0.82 mmol) was added to a stirred solution of an HCl salt of Intermediate 32 (63 mg, 0.17 mmol), succinyl dichloride (0.019 mL, 0.17 mmol) in DCM (1 mL) and the reaction mixture was stirred at rt ON. The reaction mixture was concentrated, dissolved into MeOH, filtered and purified by preparative HPLC to afford the title compound (10.1 mg). LC-MS retention time=2.37 min; m/z=775.0 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (500 MHZ, METHANOL-d₄) δ 7.39-6.86 (m, 8H), 6.79 (t, J=8.9 Hz, 2H), 6.52 (d, J=6.7 Hz, 4H), 5.87-5.76 (m, 2H), 5.14-5.05 (m, 4H), 4.31-4.16 (m, 4H), 3.84 (s, 6H), 2.97 (dd, J=13.4, 6.1 Hz, 2H), 2.75 (dd, J=13.4, 8.2 Hz, 2H), 2.41 (s, 4H) (a 2H peak is hidden under solvent).

### Example 171

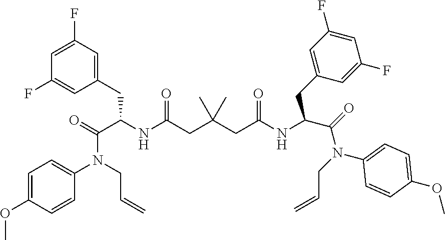 [SMILES: C=CCN(C(=O)[C@H](Cc1cc(F)cc(F)c1)NC(=O)CC(C)(C)CC(=O)N[C@@H](Cc1cc(F)cc(F)c1)C(=O)N(CC=C)c1ccc(OC)cc1)c1ccc(OC)cc1]

HATU (63.2 mg, 0.166 mmol) was added to a stirred mixture of an HCl salt of Intermediate 32 (53 mg, 0.138 mmol) and 3,3-dimethylpentanedioic acid (22.17 mg, 0.138 mmol) in DMF (1 mL) and DIPEA (0.097 mL, 0.554 mmol) and the reaction mixture was stirred at rt ON. The reaction was concentrated, dissolved into MeOH, filtered and purified by preparative HPLC to afford the title compound (21.8 mg). LC-MS retention time=2.74 min; m/z=817.1 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm). ¹H NMR (500 MHZ, MeOH-d₄) δ 7.36 (br. s., 4H), 7.10 (d, J=8.2 Hz, 4H), 6.77 (t, J=8.7 Hz, 2H), 6.52 (d, J=7.0 Hz, 4H), 5.91 (qd, J=11.0, 5.8 Hz, 2H), 5.24-5.15 (m, 4H), 4.65 (dd, J=10.4, 3.4 Hz, 2H), 4.44 (dd, J=15.0, 5.5 Hz, 2H), 4.28 (dd, J=14.8, 6.0 Hz, 2H), 3.89 (s, 6H), 3.03 (dd, J=13.7, 3.4 Hz, 2H), 2.89-2.80 (m, 2H), 2.27 (d, J=13.1 Hz, 2H), 1.93 (d, J=13.4 Hz, 2H), 1.06 (s, 6H)

### Example 172

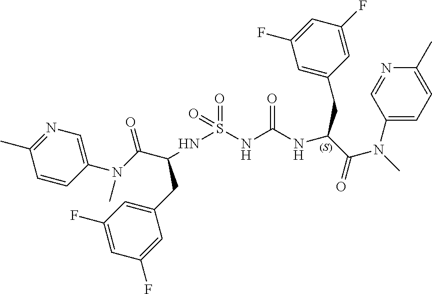

To a solution of sulfurisocyanatidic chloride (34 mg, 0.24 mmol) in DCM (1 mL) in an ice-water bath was added a solution of an HCl salt of Intermediate 102 (75 mg, 0.20 mmol) and TEA (0.10 mL, 0.60 mmol) in DCM (1 mL) over 2 min and then the reaction mixture was stirred for 20 min. A solution of an HCl salt of 2,3-dihydro-1H-pyrrolo[2,3-b]pyridine (36 mg, 0.30 mmol) in DCM (1 mL) was added, followed by TEA (0.10 mL, 0.79 mmol), the ice-water bath was removed and the reaction mixture stirred at rt for 2 h. The solvent was evaporated and the residue was redissolved in methanol and purified by preparative HPLC to afford the title compound (14.5 mg). LC-MS retention time=3.33 min; m/z=716.17 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Example 173

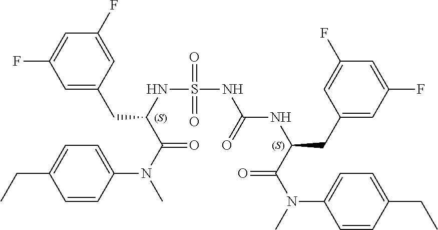

To a stirred solution of Intermediate BB-28.2 (100 mg, 0.31 mmol) in DCM (12 mL) was added TEA (0.09 mL, 0.6 mmol) followed by sulfurisocyanatidic chloride (0.067 mL, 0.47 mmol) at 0° C. and the reaction mixture was stirred at room temperature for 16 h. The reaction mixture was diluted with NaHCO₃ solution (20 mL) and extracted with DCM (3×20 mL). The combined organic layer was washed with water (20 mL), brine (20 mL), dried (Na₂SO₄), filtered and concentrated. The crude material was purified by preparative HPLC to afford the title product (10 mg) as an off white solid. LCMS retention time=2.60 min; m/z=742.2 [M+H]⁺. Column: Ascentis Express C18 (50×2.1) mm, 2.7 μm; Flow: 1.1 mL/min; Mobile Phase A: 10 mM NH₄OAc in water: ACN (95:5); Mobile Phase B: 10 mM NH₄OAc in water: ACN (5:95); Temperature: 50° C.; 0% B to 100% B over 3 minutes; UV Detection at 220 nm. ¹H NMR (400 MHz, DMSO-d₆) δ 7.83-7.67 (br. s, 1H), 7.35-7.18 (m, 8H), 7.05-6.90 (m, 2H), 6.48 (d, J=6.0 Hz, 2H), 6.31 (d, J=7.0 Hz, 2H), 4.51-4.43 (m, 1H), 4.15 (br. s., 1H), 3.13 (s, 3H), 3.02 (s, 3H), 2.79 (dd, J=13.6, 5.0 Hz, 1H), 2.73-2.56 (m, 7H), 1.23 (t, J=7.5 Hz, 3H), 1.16 (t, J=7.5 Hz, 3H).

### Example 174

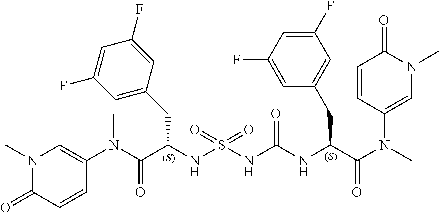

To a stirred solution of Intermediate BB-22 (0.070 g, 0.22 mmol) in DCM (2 mL) at 0° C. was added DIPEA (0.190 mL, 1.089 mmol) followed by sulfurisocyanatidic chloride (0.018 g, 0.131 mmol) and stirred at room temperature for 16 h. The reaction mixture was concentrated to dryness; the crude product was purified by preparative HPLC to afford the title product (6 mg) as an off white solid. LC-MS retention time=1.39 min; m/z=748.2 [M+H]⁺. Column: KINETIX XB-C18, 75×3 mm, 2.6 μm; Flow rate: 1 mL/min; Mobile Phase A: 10 mM HCOONH₄ in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm.

### Example 175

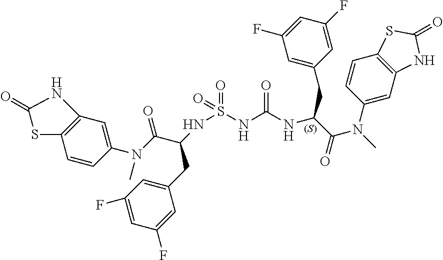

To a solution of sulfurisocyanatidic chloride (28 mg, 0.20 mmol) in DCM (1 mL), in an ice-water bath, was added a solution of an HCl salt of Intermediate 105 (65 mg, 0.16 mmol) and TEA (0.05 mL, 0.3 mmol) in DCM (1 mL) and the reaction mixture was stirred for 20 min. Then a solution of 2,3-dihydro-1H-pyrrolo[2,3-b]pyridine (29 mg, 0.24 mmol) in DCM (1 mL) was added, followed by TEA (0.10 mL, 0.65 mmol), the ice-water bath was removed and the reaction mixture stirred at rt for 2 h. The solvent was evaporated and the residue was redissolved in methanol and purified by preparative HPLC to afford the title compound (5.2 mg). LC-MS retention time=3.97 min; m/z=832.17 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Example 176

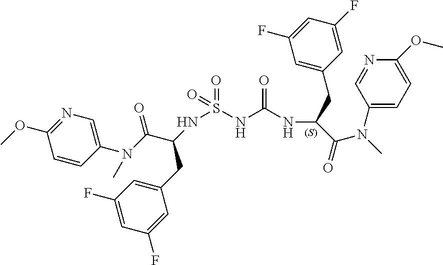

To a solution of sulfurisocyanatidic chloride (32 mg, 0.23 mmol) in DCM (1 mL) was added a solution of an HCl salt of Intermediate 107 (75 mg, 0.19 mmol) and TEA (0.08 mL, 0.6 mmol) in DCM (1 mL) in an ice-water bath. Then a solution of 2,3-dihydro-1H-pyrrolo[2,3-b]pyridine (34 mg, 0.29 mmol) in DCM (1 mL) was added, followed by TEA (0.1 mL, 0.8 mmol), the ice-water bath was removed and the reaction mixture was stirred at rt for 2 h. The solvent was evaporated and the residue was redissolved in methanol and purified by preparative HPLC to afford the title compound (12.6 mg). LC-MS retention time=4.05 min; m/z=748.17 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Example 177

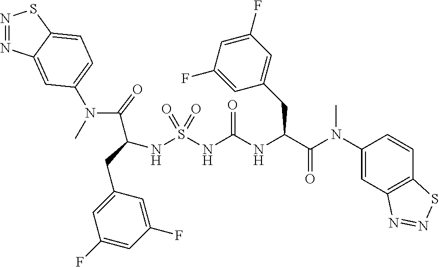 [SMILES: CN(C(=O)[C@H](Cc1cc(F)cc(F)c1)NC(=O)NS(=O)(=O)N[C@@H](Cc1cc(F)cc(F)c1)C(=O)N(C)c1ccc2snnc2c1)c1ccc2snnc2c1]

To a slurry of an HCl salt of Intermediate 82 (35 mg, 0.091 mmol) in DCM (1 mL) was added TEA (0.038 mL, 0.27 mmol). The resulting solution was then treated with sulfurisocyanatidic chloride (4.0 μL, 0.045 mmol) dropwise. The final solution was stirred at rt for 2 h. The solvent was removed in vacuo, the residue was taken up into 0.8 ml DMF, filtered and purified by preparative HPLC to afford the title compound (15.4 mg) as a white solid. LC-MS retention time=1.29 min; m/z=802.00 [M+H]+ (Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7 μm, Oven Temp.=40° C.).

### Example 178

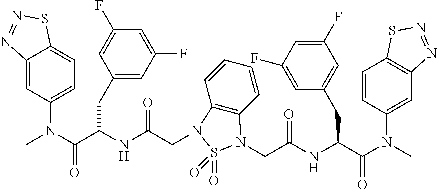 [SMILES: CN(C(=O)[C@H](Cc1cc(F)cc(F)c1)NC(=O)CN1c2ccccc2N(CC(=O)N[C@@H](Cc2cc(F)cc(F)c2)C(=O)N(C)c2ccc3snnc3c2)S1(=O)=O)c1ccc2snnc2c1]

To a solution of an HCl salt of Intermediate 82 (35 mg, 0.091 mmol), Intermediate 51 (12.3 mg, 0.043 mmol) and DIPEA (0.060 mL, 0.34 mmol) in DMF (0.5 mL) was added HATU (34 mg, 0.090 mmol) and the reaction mixture was stirred at rt for 3 h. The reaction mixture was purified by preparative HPLC to afford the title compound (21 mg) as a white solid. LC-MS retention time=1.34 min; m/z=947.00 [M+H]⁺ (Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7μm, Oven Temp.=40° C.).

### Example 179

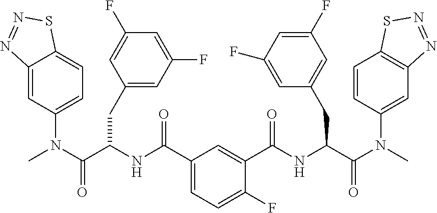 [SMILES: CN(C(=O)[C@H](Cc1cc(F)cc(F)c1)NC(=O)c1ccc(F)c(C(=O)N[C@@H](Cc2cc(F)cc(F)c2)C(=O)N(C)c2ccc3snnc3c2)c1)c1ccc2snnc2c1]

To a solution of an HCl salt of Intermediate 82 (35 mg, 0.091 mmol), 4-fluoroisophthalic acid (8.0 mg, 0.043 mmol) and DIPEA (0.061 mL, 0.35 mmol) in DMF (0.5 mL) was added HATU (35 mg, 0.091 mmol) and the reaction mixture was stirred at rt for 3 h. The reaction mixture was purified by preparative HPLC to afford the title compound (20 mg) as a white solid. LC-MS retention time=1.36 min; m/z=845.00 [M+H]+ (Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7μm, Oven Temp.=40° C.).

### Example 180

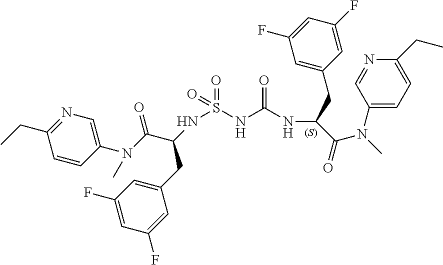

To a solution of sulfurisocyanatidic chloride (22 mg, 0.15 mmol) in DCM (1 mL) was added a solution of an HCl salt of Intermediate 110 (50 mg, 0.13 mmol) and TEA (0.05 mL, 0.38 mmol) in DCM (1 mL) in an ice-water bath, and the reaction mixture was stirred for 20 min. Then a solution of 2,3-dihydro-1H-pyrrolo[2,3-b]pyridine (23 mg, 0.19 mmol) in DCM (1 mL) was added, followed by TEA (0.07 mL, 0.51 mmol), stirred for 2 min and then the ice-water bath was removed and the reaction mixture stirred at rt for 2 h. The solvent was evaporated and the residue was redissolved in DMF and purified by preparative HPLC to afford the title compound (5.2 mg). LC-MS retention time=3.66 min; m/z=744.16 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Example 181

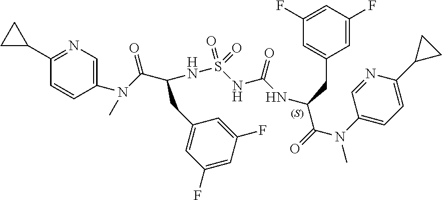

To a solution of sulfurisocyanatidic chloride (21 mg, 0.15 mmol) in DCM (1 mL) was added a solution of an HCl salt of Intermediate 91 (50 mg, 0.12 mmol) and TEA (0.05 mL, 0.37 mmol) in DCM (1 mL) in an ice-water bath and the reaction mixture was stirred for 20 min. A solution of 2,3-dihydro-1H-pyrrolo[2,3-b]pyridine (22 mg, 0.18 mmol) in DCM (1 mL) was added, followed by TEA (0.07 mL, 0.49 mmol), stirred for 2 min, the ice-water bath was removed and the reaction mixture was stirred at rt for 2 h. The solvent was evaporated and the residue was redissolved in DMF and purified by preparative HPLC to afford the title compound (14.1 mg). LC-MS retention time=3.83 min; m/z=768.17 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Example 182

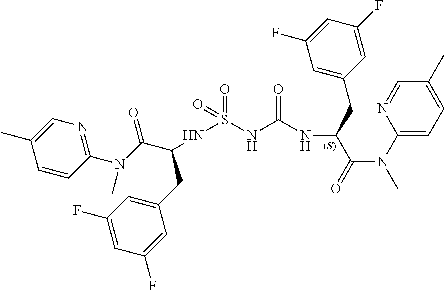

To a solution of sulfurisocyanatidic chloride (22 mg, 0.16 mmol) in DCM (1 mL) was added a mixture of an HCl salt of Intermediate 112 (50 mg, 0.13 mmol) and TEA (0.05 mL, 0.39 mmol) in DCM (1 mL) in an ice-water bath and the reaction mixture was stirred for 20 min. Then a solution of 2,3-dihydro-1H-pyrrolo[2,3-b]pyridine (24 mg, 0.20 mmol) in DCM (1 mL) was added, followed by TEA (0.1 mL, 0.5 mmol), stirred for 3 min, the ice-water bath was removed and the reaction mixture was stirred at rt for 1.5 h. The solvent was evaporated and the residue was redissolved in DMF and purified by preparative HPLC to afford the title compound (5.4 mg). LC-MS retention time=4.09 min; m/z=716.15 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Example 182B

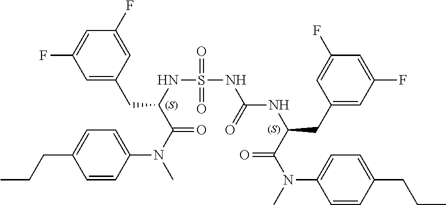

To a stirred solution of Intermediate 29.2 (100 mg, 0.3 mmol) in DCM (10 mL) was added chlorosulfonyl isocyanate (0.04 mL, 0.45 mmol), TEA (0.2 mL, 1.50 mmol) at 0° C. and the reaction mixture was stirred for 30 min. Then added 2,3-dihydro-1h-pyrrolo[2,3-b]pyridine (54.2 mg, 0.45 mmol) in DCM (10 mL) followed by TEA (0.21 mL, 1.50 mmol) and the reaction mixture stirred at room temperature for 2 h. The reaction mixture was concentrated to dryness; the crude material was purified by preparative HPLC to afford the title compound (3.2 mg; off white solid). Title compound: LC-MS retention time=2.8 min; m/z=770.3 [M+H]⁺. Column: Ascentis Express C18 (50×2.1) mm, 2.7 μm; Flow: 1.1 mL/min; Mobile Phase A: 10 mM NH₄OAc in water: ACN (95:5); Mobile Phase B: 10 mM NH₄OAc in water: ACN (5:95); Temperature: 50° C.; 0% B to 100% B over 3 minutes; UV Detection at 220 nm.

### Example 183

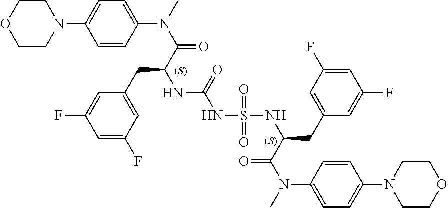

To a solution of Intermediate BB-25 (150 mg, 0.40 mmol) in DCM (10 mL) was added DIPEA (0.35 mL, 2.0 mmol), sulfurisocyanatidic chloride (0.020 mL, 0.24 mmol) and stirred the reaction mixture at room temperature for 20 min. The reaction mixture was diluted with DCM (20 mL), washed with water (20 mL), dried (Na₂SO₄), filtered, concentrated and the crude product was purified by preparative HPLC to afford the title product (7 mg) as pale yellow solid. LC-MS retention time=2.07 min; m/z=856.2 [M+H]⁺. Column: Ascentis Express C18 (50×2.1) mm, 2.7 μm; Flow: 1.1 mL/min; Mobile Phase A: 10 mM NH₄OAc in water: ACN (95:5); Mobile Phase B: 10 mM NH₄OAc in water: ACN (5:95); Temperature: 50° C.; 0% B to 100% B over 3 minutes; UV Detection at 220 nm.

### Example 184

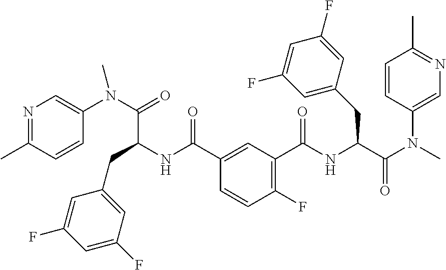 [SMILES: Cc1ccc(N(C)C(=O)[C@H](Cc2cc(F)cc(F)c2)NC(=O)c2ccc(F)c(C(=O)N[C@@H](Cc3cc(F)cc(F)c3)C(=O)N(C)c3ccc(C)nc3)c2)cn1]

To a solution of an HCl salt of Intermediate 102 (52 mg, 0.14 mmol) and 4-fluoroisophthalic acid (12 mg, 0.06 mmol) in DMF (1 mL) was added DIPEA (0.07 mL, 0.4 mmol) and then HATU (50 mg, 0.13 mmol) and the reaction mixture was stirred at rt for 2 h. The solvent was evaporated and the residue was redissolved in DMF and purified by preparative HPLC to afford the title compound (35.4 mg). LC-MS retention time=3.51 min; m/z=759.38 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Example 185

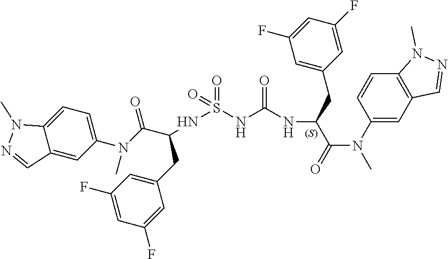

A solution of sulfurisocyanatidic chloride (20 mg, 0.14 mmol) in DCM (1 mL) was added slowly to a mixture of Intermediate 114 (50 mg, 0.12 mmol) and TEA (0.10 mL, 0.47 mmol) in DCM (1 mL) in an ice-water bath and the reaction mixture was stirred for 20 min. Then a solution of 2,3-dihydro-1H-pyrrolo[2,3-b]pyridine (21.6 mg, 0.18 mmol) in DCM (1 mL) was added and followed by TEA (0.07 mL, 0.5 mmol) and the reaction mixture was stirred for 5 min, the bath was removed and the stirring was continued for 2 h. It was concentrated and the residue was redissolved in methanol and purified by preparative HPLC to afford the title compound (14.9 mg). LC-MS retention time=3.96 min; m/z=794.35 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Example 186

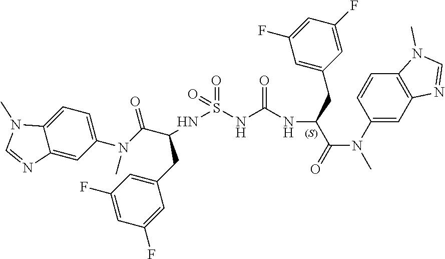

To a solution of sulfurisocyanatidic chloride (27 mg, 0.19 mmol) in DCM (1 mL) in an ice-water bath was added slowly a mixture of an HCl salt of Intermediate 116 (65 mg, 0.16 mmol) and TEA (0.1 mL, 0.47 mmol) in DCM (1 mL) and the reaction mixture was stirred for 20 min. A solution of 2,3-dihydro-1H-pyrrolo[2,3-b]pyridine (28 mg, 0.23 mmol) in DCM (1 mL) was added, followed by TEA (0.1 mL, 0.62 mmol). The reaction mixture was stirred for 5 min and the ice-water bath was removed and the reaction mixture was stirred at rt for 2 h. The solvent was evaporated and the residue was redissolved in methanol and purified by preparative HPLC to afford the title compound (9.9 mg). LC-MS retention time=3.07 min; m/z=794.3 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Example 187

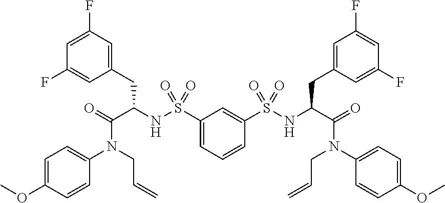

DMF (0.75 μl, 9.7 μmol) was added to a mixture of sodium benzene-1,3-disulfonate (55%) (50 mg, 0.097 mmol) and SOCl₂ (2.00 mL, 27.4 mmol) and the reaction mixture was stirred at 80° C. for 1 d and then concentrated under vacum. To the residue was added DCM (1 mL), and an HCl salt of Intermediate 32 (74.6 mg, 0.19 mmol), followed by TEA (0.068 mL, 0.487 mmol) and the mixture was stirred at rt for 16 h. Additional HCl salt of Intermediate 32 (50 mg) was added and the reaction mixture was stirred for 3 h. The reaction mixture was partitioned between EtOAc (2×20 mL) and water and the combined organic components were concentrated and purified by preparative HPLC to afford the title compound (62.1 mg). LC-MS retention time=2.45 min; m/z=895.0 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Example 188

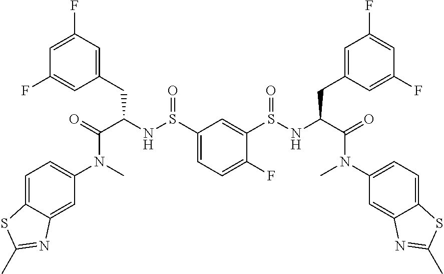

To a mixture of an HCl salt of Intermediate 73 (47 mg, 0.11 mmol) and 4-fluoroisophthalic acid (9.9 mg, 0.054 mmol) in DMF (1 mL) and DIPEA (0.057 mL, 0.32 mmol), HATU (43.2 mg, 0.11 mmol) was added and the reaction mixture was stirred at rt for 3 h. The reaction was filtered, and purified by preparative HPLC to afford the title compound (20.7 mg). LC-MS retention time=2.68 min; m/z=871.0 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm). ¹H NMR (500 MHZ, DMSO-d₆) δ 8.94 (d, J=7.7 Hz, 1H), 8.83 (d, J=7.0 Hz, 1H), 8.17 (t, J=7.5 Hz, 2H), 8.01 (br. s., 3H), 7.93 (br. s., 1H), 7.44 (d, J=7.0 Hz, 2H), 7.37 (t, J=9.2 Hz, 1H), 7.04-6.92 (m, 2H), 6.49 (dd, J=14.7, 7.0 Hz, 4H), 4.73-4.55 (m, 2H), 3.27 (br. s., 2H), 3.09-2.94 (m, 3H), 2.90 (s, 3H), 2.83 (s, 6H), 2.74 (s, 2H)

### Example 189

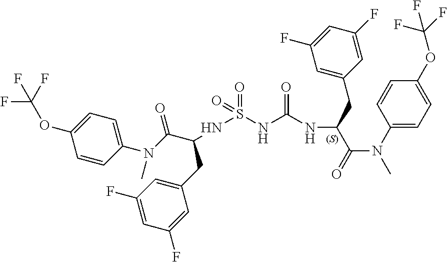

To a solution of sulfurisocyanatidic chloride (25 mg, 0.18 mmol) in DCM (1 mL) was added a mixture of an HCl salt of Intermediate 118 (60 mg, 0.15 mmol) and TEA (0.04 mL, 0.29 mmol) in DCM (1 mL) over 2 min in an ice-water bath and the reaction mixture was stirred for 20 min. A solution of 2,3-dihydro-1H-pyrrolo[2,3-b]pyridine (26 mg, 0.22 mmol) in DCM (1 mL) was added, followed by TEA (0.08 mL, 0.6 mmol), the ice-water bath was removed and the reaction mixture was stirred for 2 h. The solvent was evaporated and the residue was redissolved in methanol and purified by preparative HPLC to afford the title compound (12.9 mg). LC-MS retention time=4.56 min; m/z=854.29 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Example 190

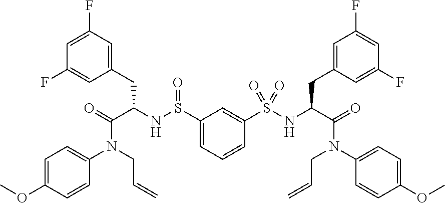

To a mixture of 3-(chlorosulfonyl)benzoyl chloride (50 mg, 0.21 mmol) and an HCl salt of Intermediate 32 (160 mg, 0.42 mmol) in DCM (2 mL), TEA (0.15 mL, 1.0 mmol) was added and the reaction mixture was stirred at rt for 3 h. The reaction was filtered, and purified by preparative HPLC to afford the title compound (58.5 mg). LC-MS retention time=2.88 min; m/z=859.0 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Example 191

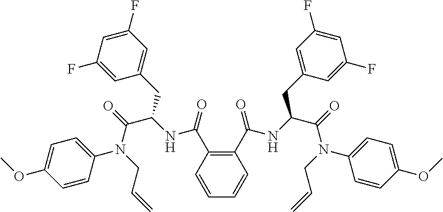

To a mixture of phthaloyl dichloride (15.9 mg, 0.078 mmol) and an HCl salt of Intermediate 32 (60 mg, 0.15 mmol) in DCM (1 mL), TEA (0.06 mL, 0.4 mmol) was added and the reaction mixture was stirred at rt for 1 d. The reaction was filtered, and purified by preparative HPLC to afford the title compound (14.9 mg). LC-MS retention time=2.91 min; m/z=823.1 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Example 192

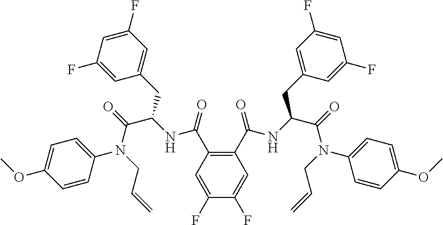

To a mixture of 4,5-difluorophthalic acid (15.8 mg, 0.078 mmol) and an HCl salt of Intermediate 32 (60 mg, 0.16 mmol) in DMF (1 mL) and DIPEA (0.068 mL, 0.39 mmol), HATU (62.6 mg, 0.16 mmol) was added and the reaction mixture was stirred at rt for 3 h. The reaction was filtered, and purified by preparative HPLC to afford the title compound (52 mg). LC-MS retention time=2.60 min; m/z=859.2 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm). ¹H NMR (500 MHZ, MeOH-d₄) δ 7.51 (t, J=9.0 Hz, 2H), 6.93-6.79 (m, 6H), 7.02-6.77 (m, 1H), 6.69-6.56 (m, 1H), 6.64 (d, J=6.6 Hz, 4H), 5.82-5.72 (m, 2H), 5.13-4.99 (m, 4H), 4.92-4.81 (m, 2H), 4.22 (dd, J=14.9, 6.1 Hz, 2H), 4.08 (dd, J=14.9, 6.1 Hz, 2H), 3.77 (s, 6H), 3.31-3.19 (m, 2H), 3.08-2.98 (m, 2H), 2.90 (dd, J=13.0, 6.4 Hz, 2H).

### Example 193

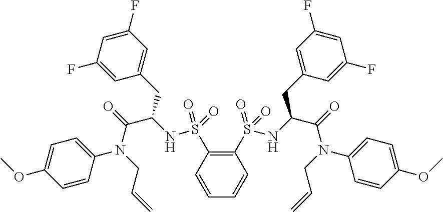

To a mixture of benzene-1,2-disulfonyl dichloride (21.5 mg, 0.078 mmol) and an HCl salt of Intermediate 32 (60 mg, 0.16 mmol) in DCM (1 mL), TEA (0.055 mL, 0.39 mmol) was added and the reaction mixture was stirred at rt for 1 d. The reaction was filtered and purified by preparative HPLC to afford the title compound (12.4 mg). LC-MS retention time=2.95 min; m/z=895.0 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Example 194

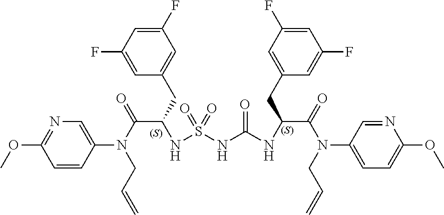

To a stirred solution of Intermediate BB-33.2 (80 mg, 0.23 mmol) and triethylamine (0.06 mL, 0.46 mmol) in DCM (5 mL) at 0° C. was added dropwise chlorosulfonyl isocyanate (10 μL, 0.115 mmol) and the reaction mixture was stirred at the same temperature for 2 h. The reaction mixture was diluted with DCM (˜10 mL), washed with water (10 mL) and brine (10 mL), dried (Na₂SO₄), filtered and concentrated. The crude product was purified by combiflash chromatography (12 g Redisep® SiO₂ column, eluting with 1.5-2.0% MeOH in chloroform) to afford the title compound (75 mg) as an off-white solid. LC-MS retention time=2.99 min; m/z=800.2 [M+H]⁺. Column: KINETIX C18, 75×3 mm, 2.6 μm; Flow rate: 1 mL/min; Mobile Phase A: 10 mM HCO₂NH₄ in 98% Water/2% ACN; Mobile Phase B: 10 mM HCO₂NH₄ in 2% Water/98% ACN; 20% B to 100% B over 4 min, then hold for 0.6 min at 100% B with flow rate of 1.5 mL/min; Detection: UV at 220 nm.

### Example 195

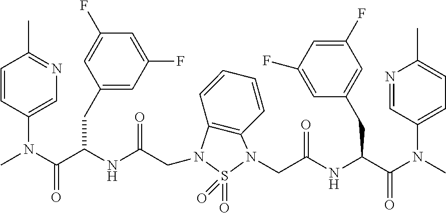 [SMILES: Cc1ccc(N(C)C(=O)[C@H](Cc2cc(F)cc(F)c2)NC(=O)CN2c3ccccc3N(CC(=O)N[C@@H](Cc3cc(F)cc(F)c3)C(=O)N(C)c3ccc(C)nc3)S2(=O)=O)cn1]

To a solution of an HCl salt of Intermediate 102 (38.8 mg, 0.103 mmol), Intermediate 51 (14 mg, 0.049 mmol) and DIPEA (0.068 mL, 0.39 mmol) in DMF (0.5 mL) was added HATU (39 mg, 0.10 mmol) and the reaction mixture was stirred at rt for 3 h. The reaction mixture was purified by preparative HPLC to afford the title compound (34 mg) as a white solid. LC-MS retention time=0.95 min; m/z=861.00 [M+H]+ (Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7μm, Oven Temp.=40° C.).

### Example 196

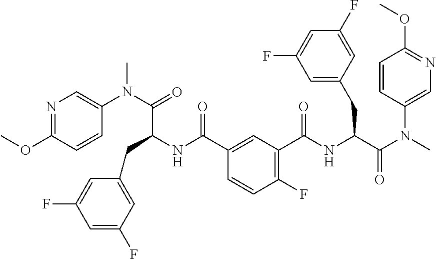 [SMILES: COc1ccc(N(C)C(=O)[C@H](Cc2cc(F)cc(F)c2)NC(=O)c2ccc(F)c(C(=O)N[C@@H](Cc3cc(F)cc(F)c3)C(=O)N(C)c3ccc(OC)nc3)c2)cn1]

To a solution of an HCl salt of Intermediate 107 (52 mg, 0.13 mmol) and 4-fluoroisophthalic acid (11 mg, 0.06 mmol) in DMF (1 mL) was added DIPEA (0.06 mL, 0.4 mmol) and then HATU (48 mg, 0.13 mmol) and the reaction mixture was stirred at rt for 2 h. The solvent was evaporated and the residue was redissolved in methanol and purified by preparative HPLC to afford the title compound (18.1 mg). LC-MS retention time=4.23 min; m/z=791.31 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Example 197

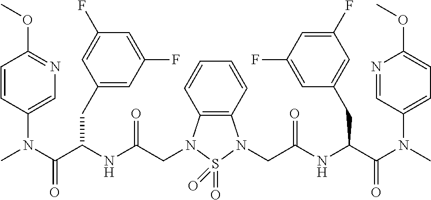 [SMILES: COc1ccc(N(C)C(=O)[C@H](Cc2cc(F)cc(F)c2)NC(=O)CN2c3ccccc3N(CC(=O)N[C@@H](Cc3cc(F)cc(F)c3)C(=O)N(C)c3ccc(OC)nc3)S2(=O)=O)cn1]

To a solution of Intermediate 51 (19 mg, 0.07 mmol) and an HCl salt of Intermediate 107 (55 mg, 0.14 mmol) in DMF (1 mL) was added DIPEA (0.10 mL, 0.53 mmol) and then HATU (53 mg, 0.14 mmol) and the reaction mixture was stirred at rt for 2 h. The solvent was evaporated and the residue was redissolved in methanol and purified by preparative HPLC to afford the title compound (19 mg). LC-MS retention time=4.20 min; m/z=893.35 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Example 198

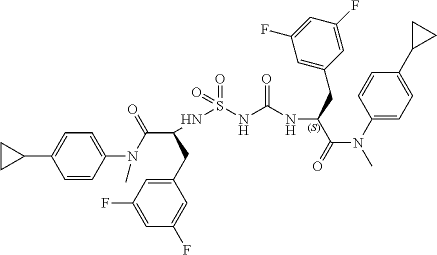

To a solution of sulfurisocyanatidic chloride (35 mg, 0.25 mmol) in DCM (2 mL) was added a solution of an HCl salt of Intermediate 120 (100 mg, 0.25 mmol) and TEA (0.10 mL, 0.74 mmol) in DCM (2 mL) in an ice-water bath and the reaction mixture was stirred for 20 min. A solution of 2,3-dihydro-1H-pyrrolo[2,3-b]pyridine (45 mg, 0.37 mmol) in DCM (2 mL) was added, followed by TEA (0.14 mL, 0.99 mmol). The reaction mixture was stirred for 2 min, the ice-water bath was removed and the reaction mixture was stirred at rt for 2 h. The solvent was evaporated and the residue was redissolved in methanol and purified by preparative HPLC to afford the title compound (26.2 mg). LC-MS retention time=4.56 min; m/z=766.32 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Example 199

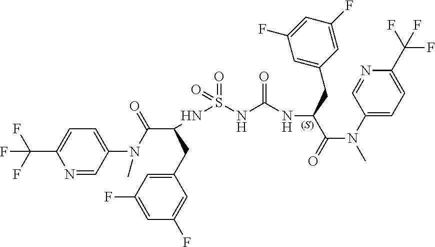

To a solution of sulfurisocyanatidic chloride (36 mg, 0.25 mmol) in DCM (2 mL) was added a solution of an HCl salt of Intermediate 128 (90 mg, 0.25 mmol) and TEA (0.05 mL, 0.4 mmol) in DCM (2 mL) in an ice-water bath and the reaction mixture was stirred for 20 min. Then a solution of 2,3-dihydro-1H-pyrrolo[2,3-b]pyridine (45.1 mg, 0.376 mmol) in DCM (2 mL) was added, followed by TEA (0.10 mL, 0.75 mmol) and the reaction mixture was stirred for 2 min and then the ice-water bath was removed and the stirring continued at rt for 2 h. The solvent was evaporated and the residue was redissolved in methanol and purified by preparative HPLC to afford the title compound (6.9 mg). LC-MS retention time=4.14 min; m/z=824.22 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Example 200

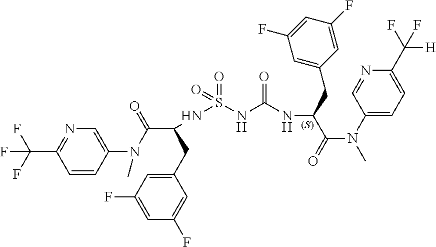

To a solution of sulfurisocyanatidic chloride (21 mg, 0.15 mmol) in DCM (1 mL) in an ice-water bath was added a solution of an HCl salt of Intermediate 131 (50 mg, 0.15 mmol) and TEA (0.02 mL, 0.2 mmol) in DCM (1 mL) and the reaction mixture was stirred for 2 min. Then a solution of 2,3-dihydro-1H-pyrrolo[2,3-b]pyridine (26 mg, 0.22 mmol) in DCM (1 mL) was added, followed by TEA (0.04 mL, 0.3 mmol), the ice-water bath was removed and the reaction mixture was stirred at rt for 2 h. The solvent was evaporated and the residue was redissolved in methanol and purified by preparative HPLC to afford the title compound (6.0 mg). LC-MS retention time=3.76 min; m/z=788.26 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Example 201

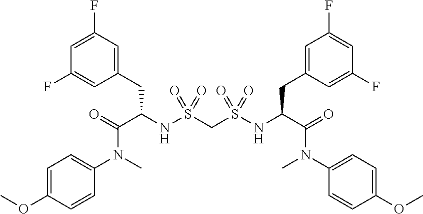

An HCl salt of Intermediate 13 (30 mg, 0.084 mmol) and methanedisulfonyl dichloride (9.0 mg, 0.042 mmol) were dissolved into DCM (3 mL) and then treated with TEA (0.03 mL, 0.2 mmol). The reaction mixture was then stirred at rt ON. The reaction mixture was treated with additional methanedisulfonyl dichloride (9.0 mg, 0.042 mmol) and TEA (0.03 mL, 0.2 mmol) and stirring was continued at rt for 5 h. The reaction mixture was concentrated, and the residue was dissolved into MeOH, filtered and purified by preparative HPLC to afford the title compound (9.3 mg). LC-MS retention time=2.43 min; m/z=781.1 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Example 202

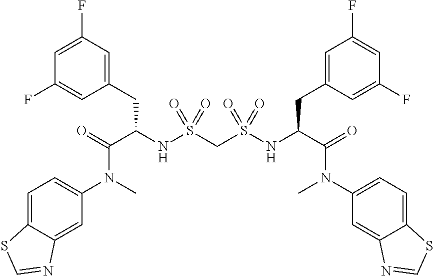 [SMILES: CN(C(=O)[C@H](Cc1cc(F)cc(F)c1)NS(=O)(=O)CS(=O)(=O)N[C@@H](Cc1cc(F)cc(F)c1)C(=O)N(C)c1ccc2scnc2c1)c1ccc2scnc2c1]

An HCl salt of Intermediate 20 (32.3 mg, 0.084 mmol) and methanedisulfonyl dichloride (9.0 mg, 0.042 mmol) were dissolved into DCM (3 mL) and then treated with TEA (0.03 mL, 0.2 mmol). The reaction mixture was then stirred at rt ON. The reaction mixture was treated with additional methanedisulfonyl dichloride (9.0 mg, 0.042 mmol) and TEA (0.03 mL, 0.2 mmol) and stirring was continued at rt for 5 h. The reaction mixture was concentrated, and the residue was dissolved into MeOH, filtered and purified by preparative HPLC to afford the title compound (9.3 mg). LC-MS retention time=2.20 min; m/z=835.2 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Example 203

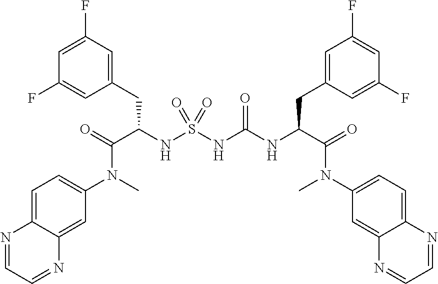 [SMILES: CN(C(=O)[C@H](Cc1cc(F)cc(F)c1)NC(=O)NS(=O)(=O)N[C@@H](Cc1cc(F)cc(F)c1)C(=O)N(C)c1ccc2nccnc2c1)c1ccc2nccnc2c1]

To a solution of Intermediate 176 (20 mg, 0.058 mmol) in DCM (1 mL) and TEA (0.016 mL, 0.12 mmol), a DCM (0.1 mL) solution of sulfurisocyanatidic chloride (4.6 mg, 0.032 mmol) was added dropwise, the mixture was stirred at rt for 2 h, then it was filtered and purified by preparative HPLC to afford the title compound (11.1 mg). LC-MS retention time=1.59 min; m/z=790.1 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Example 204

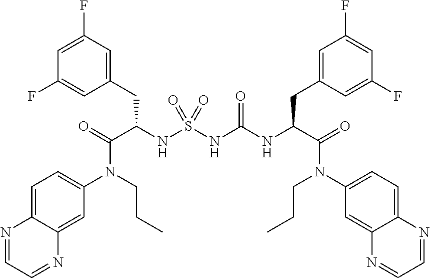 [SMILES: CCCN(C(=O)[C@H](Cc1cc(F)cc(F)c1)NC(=O)NS(=O)(=O)N[C@@H](Cc1cc(F)cc(F)c1)C(=O)N(CCC)c1ccc2nccnc2c1)c1ccc2nccnc2c1]

To a solution of Intermediate 181 (19 mg, 0.051 mmol) in DCM (1 mL) and TEA (0.014 mL, 0.1 mmol), a DCM (0.1 mL) solution of sulfurisocyanatidic chloride (4.0 mg, 0.028 mmol) was added dropwise, the mixture was stirred at rt for 2 h, then it was filtered and purified by preparative HPLC to afford the title compound (5.1 mg). LC-MS retention time=1.96 min; m/z=846.0 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Example 205

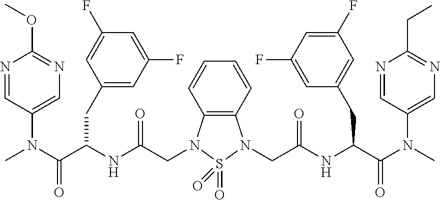 [SMILES: CCc1ncc(N(C)C(=O)[C@H](Cc2cc(F)cc(F)c2)NC(=O)CN2c3ccccc3N(CC(=O)N[C@@H](Cc3cc(F)cc(F)c3)C(=O)N(C)c3cnc(OC)nc3)S2(=O)=O)cn1]

To a solution of an HCl salt of Intermediate 83 (39 mg, 0.099 mmol), Intermediate 51 (13.5 mg, 0.0470 mmol) and DIPEA (0.066 mL, 0.38 mmol) in DMF (0.5 mL) was added HATU (38 mg, 0.099 mmol). The resulting mixture was stirred at rt ON and purified by preparative HPLC to afford the title compound (34 mg) as a white solid. LC-MS retention time=1.23 min; m/z=891.05 [M+H]+ (Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7 μm, Oven Temp.=40° C.).

### Example 206

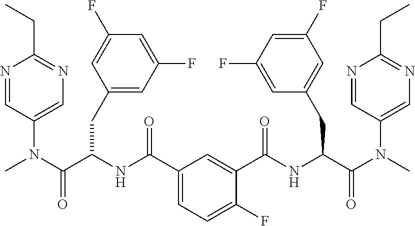 [SMILES: CCc1ncc(N(C)C(=O)[C@H](Cc2cc(F)cc(F)c2)NC(=O)c2ccc(F)c(C(=O)N[C@@H](Cc3cc(F)cc(F)c3)C(=O)N(C)c3cnc(CC)nc3)c2)cn1]

To a solution of an HCl salt of Intermediate 83 (39 mg, 0.099 mmol), 4-fluoroisophthalic acid (8.7 mg, 0.047 mmol) and DIPEA (0.066 mL, 0.38 mmol) in DMF (0.5 mL) was added HATU (38 mg, 0.099 mmol). The resulting mixture was stirred at rt for 3 h and purified by preparative HPLC to afford the title compound (29 mg) as a white solid. LC-MS retention time=1.24 min; m/z=789.10 [M+H]⁺(Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7 μm, Oven Temp.=40° C.).

### Example 207

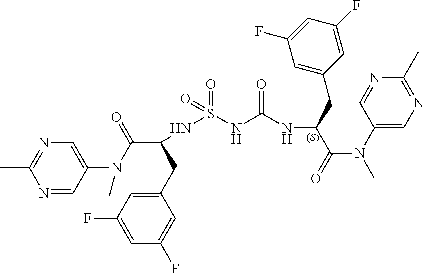

To a solution of sulfurisocyanatidic chloride (37 mg, 0.26 mmol) in DCM (2 mL) in an ice-water bath was added a solution of an HCl salt of Intermediate 133 (100 mg, 0.26 mmol) and TEA (0.11 mL, 0.79 mmol) in DCM (2 mL) and the reaction mixture was stirred for 2 min. Then a solution of 2,3-dihydro-1H-pyrrolo[2,3-b]pyridine (48 mg, 0.39 mmol) in DCM (2 mL) was added, followed by TEA (0.15 mL, 1.05 mmol), the ice-water bath was removed and the reaction mixture was stirred at rt for 2 h. The solvent was evaporated and the residue was redissolved in DMF and methanol and then purified by preparative HPLC to afford the title compound (8.8 mg). LC-MS retention time=3.96 min; m/z=718.27 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Example 208

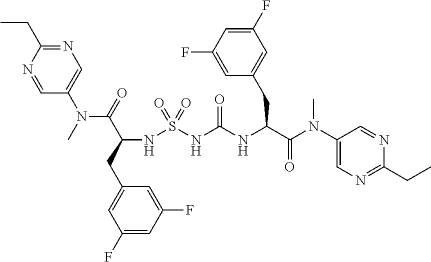 [SMILES: CCc1ncc(N(C)C(=O)[C@H](Cc2cc(F)cc(F)c2)NC(=O)NS(=O)(=O)N[C@@H](Cc2cc(F)cc(F)c2)C(=O)N(C)c2cnc(CC)nc2)cn1]

To a slurry of an HCl salt of Intermediate 83 (39 mg, 0.099 mmol) in DCM (1 mL) was added TEA (0.083 mL, 0.60 mmol). The resulting solution was then treated with sulfurisocyanatidic chloride (4.3 μL, 0.050 mmol) dropwise. The final solution was stirred at rt for 2 h. The solvent was evaporated and the residue was taken up into DMF (0.5 mL) and purified by preparative HPLC to afford the title compound (8.4 mg) as a white solid. LC-MS retention time=1.18 min; m/z=746.10 [M+H]+ (Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7 μm, Oven Temp.=40° C.).

### Example 209

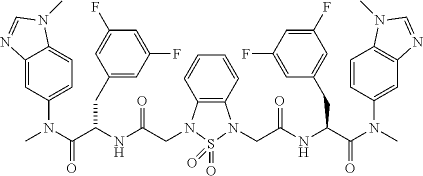 [SMILES: CN(C(=O)[C@H](Cc1cc(F)cc(F)c1)NC(=O)CN1c2ccccc2N(CC(=O)N[C@@H](Cc2cc(F)cc(F)c2)C(=O)N(C)c2ccc3c(c2)ncn3C)S1(=O)=O)c1ccc2c(c1)ncn2C]

To a solution of Intermediate 51 (6.5 mg, 0.02 mmol) and an HCl salt of Intermediate 116 (20 mg, 0.05 mmol) in DMF (1 mL) was added DIPEA (0.03 mL, 0.18 mmol) and then HATU (18 mg, 0.05 mmol) and the reaction mixture was stirred at rt for 16 h. The solvent was evaporated and the residue was redissolved in DMF and methanol and then purified by preparative HPLC to afford the title compound (7.5 mg). LC-MS retention time=3.13 min; m/z=961.41 [M+Na]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Example 210

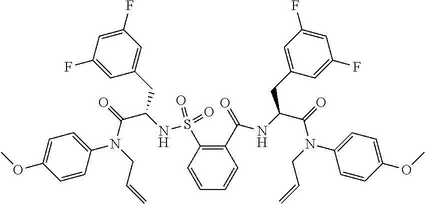

To a mixture of 2-sulfobenzoic acid (20 mg, 0.1 mmol) and SOCl₂ (1.0 mL, 14 mmol), DMF (0.8 μl, 10 μmol) was added and the reaction mixture was stirred at 80° C. for 1 d, the reaction mixture was concentrated under vacuum. The residue was azeotroped with toluene, dissolved in DCM (1 mL), treated with an HCl salt of Intermediate 32 (76 mg, 0.2 mmol), followed by TEA (0.069 mL, 0.49 mmol) and then the reaction mixture was stirred at rt for 16 h. The reaction mixture was partitioned between EtOAc (2×20 mL) and water and the combined organic components were concentrated, dissolved in DMF, and purified by preparative HPLC to afford the title compound (27.3 mg). LC-MS retention time=2.84 min; m/z=859.0 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Example 211

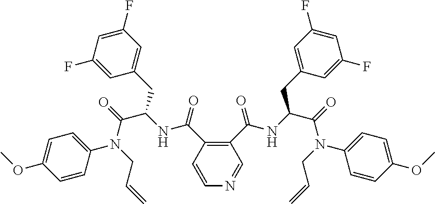

To a mixture of pyridine-3,4-dicarboxylic acid (13.1 mg, 0.078 mmol) and an HCl salt of Intermediate 32 (60 mg, 0.15 mmol) in DMF (1 mL) and DIPEA (0.068 mL, 0.39 mmol), HATU (62.6 mg, 0.16 mmol) was added and the reaction mixture was stirred at rt for 3 h. The reaction was filtered and purified by preparative HPLC to afford the title compound (49.9 mg). LC-MS retention time=2.49 min; m/z=824.1 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Example 212

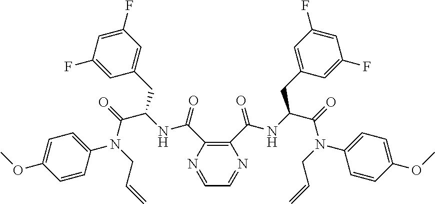

To a mixture of pyrazine-2,3-dicarboxylic acid (13.1 mg, 0.078 mmol) and an HCl salt of Intermediate 32 (60 mg, 0.15 mmol) in DMF (1 mL) and DIPEA (0.068 mL, 0.39 mmol), HATU (62.6 mg, 0.16 mmol) was added and the reaction mixture was stirred at rt for 3 h. The reaction was filtered and purified by preparative HPLC to afford the title compound (42.7 mg). LC-MS retention time=2.63 min; m/z=825.1 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Example 213

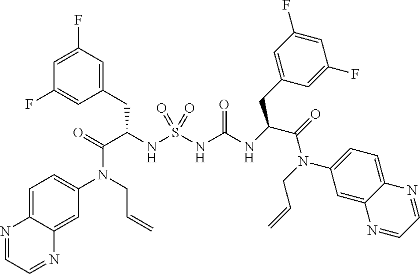 [SMILES: C=CCN(C(=O)[C@H](Cc1cc(F)cc(F)c1)NC(=O)NS(=O)(=O)N[C@@H](Cc1cc(F)cc(F)c1)C(=O)N(CC=C)c1ccc2nccnc2c1)c1ccc2nccnc2c1]

To a solution of an HCl salt of Intermediate 179 (70.6 mg, 0.16 mmol) in DCM (1 mL) and TEA (0.056 mL, 0.4 mmol), a DCM (0.3 mL) solution of sulfurisocyanatidic chloride (12.4 mg, 0.088 mmol) was added dropwise and the reaction mixture was stirred at rt for 2 h. The reaction was filtered and purified by preparative HPLC to afford the title compound (36.5 mg). LC-MS retention time=2.02 min; m/z=842.0 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Example 214

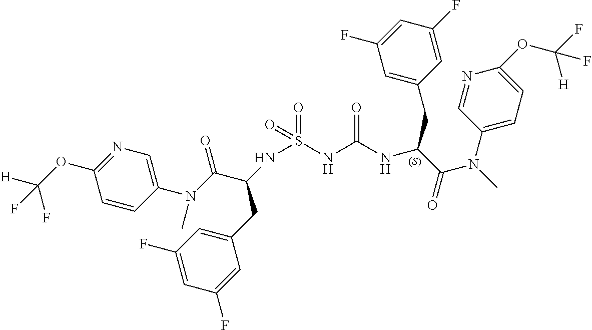

To a solution of sulfurisocyanatidic chloride (23 mg, 0.16 mmol) in DCM (2 mL) in an ice-water bath was added a solution of an HCl salt of Intermediate 142 (70 mg, 0.16 mmol) and TEA (0.07 mL, 0.49 mmol) in DCM (2 mL) and the reaction mixture was stirred for 1 min. A solution of 2,3-dihydro-1H-pyrrolo[2,3-b]pyridine (29 mg, 0.24 mmol) in DCM (2 mL) was added, followed by TEA (0.1 mL, 0.65 mmol), stirred for 3 min and the ice-water bath was removed and stirred at rt for 4 h. The solvent was evaporated and the residue was redissolved in DMF and methanol and then purified by preparative HPLC to afford the title compound (8.1 mg). LC-MS retention time=4.23 min; m/z=820.24 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Example 215

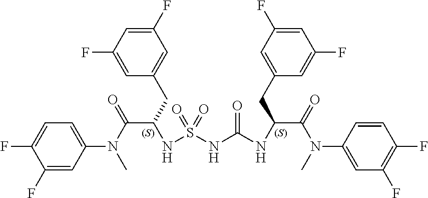

To a stirred solution of Intermediate BB-27 (150 mg, 0.46 mmol) and TEA (0.06 mL, 0.46 mmol) in DCM (5 mL) was added chlorosulfonyl isocyanate (0.05 mL, 0.6 mmol) at 0° C. and stirred at the same temperature for 2 h. The reaction mixture was diluted with DCM (20 mL), washed with water (20 mL), brine (20 mL), dried (Na₂SO₄), filtered and concentrated. The crude product was purified by preparative HPLC purification to afford the title compound (25 mg) as an off white solid. LC-MS retention time=2.31 min; m/z=756.1 [M−H]. Column: Ascentis Express C18 (50×2.1) mm, 2.7 μm; Flow: 1.1 mL/min; Mobile Phase A: 10 mM NH₄OAc in water: ACN (95:5); Mobile Phase B: 10 mM NH₄OAc in water: ACN (5:95); Temperature: 50° C.; 0% B to 100% B over 3 minutes; UV Detection at 220 nm. ¹H NMR (400 MHz, DMSO-d₆) δ 9.81 (s, 1H), 8.10 (d, J=9.0 Hz, 1H), 7.57 (dd, J=19.2, 10.4 Hz, 1H), 7.45 (dd, J=18.8, 7.2 Hz, 1H), 7.36 (m, 2H), 7.18 (br. s., 2H), 7.10-7.00 (m, 2H), 6.62 (d, J=7.2 Hz, 2H), 6.57 (d, J=8.0 Hz, 1H), 6.53 (d, J=8.0 Hz, 2H), 4.43 (m, 1H), 4.16 (m, 1H), 3.10 (s, 3H), 3.02 (s, 3H), 2.90-2.80 (m, 2H), 2.75-2.65 (m, 2H).

### Example 216

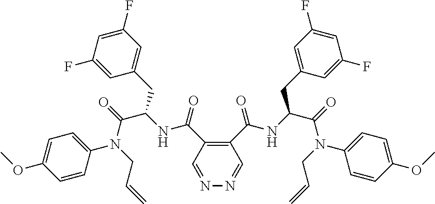

To a mixture of an HCl salt of Intermediate 32 (60 mg, 0.15 mmol) and pyridazine-4,5-dicarboxylic acid (13.1 mg, 0.078 mmol) in DMF (1 mL) and DIPEA (0.068 mL, 0.39 mmol), HATU (62.6 mg, 0.16 mmol) was added and the reaction mixture was stirred at rt for 3 h. The reaction was filtered and purified by preparative HPLC to afford the title compound (42 mg). LC-MS retention time=2.48 min; m/z=825.1 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Example 217

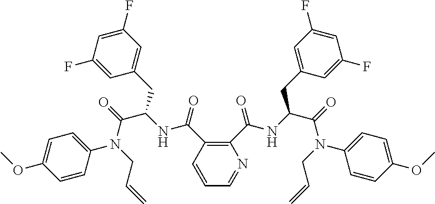

To a mixture of an HCl salt of Intermediate 32 (60 mg, 0.15 mmol) and pyridine-2,3-dicarboxylic acid (13.1 mg, 0.078 mmol) in DMF (1 mL) and DIPEA (0.068 mL, 0.39 mmol), HATU (62.6 mg, 0.16 mmol) was added and the reaction mixture was stirred at rt for 3 h. The reaction was filtered and purified by preparative HPLC to afford the title compound (41.6 mg). LC-MS retention time=2.56 min; m/z=824.1 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Example 218

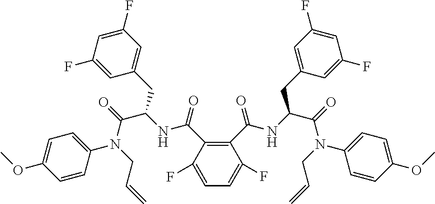

To a mixture of 3,6-difluorophthalic acid (15.8 mg, 0.078 mmol) and an HCl salt of Intermediate 32 (60 mg, 0.15 mmol) in DMF (1 mL) and DIPEA (0.068 mL, 0.39 mmol), HATU (62.6 mg, 0.16 mmol) was added and the reaction mixture was stirred at rt for 3 h. The reaction was filtered and purified by preparative HPLC to afford the title compound (39.8 mg). LC-MS retention time=2.60 min; m/z=859.1 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Example 219

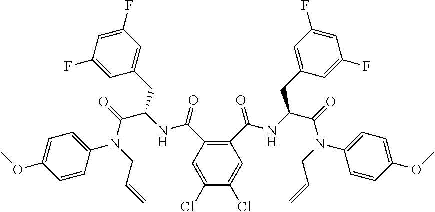

To a mixture of 4,5-dichlorophthalic acid (18.4 mg, 0.078 mmol) and an HCl salt of Intermediate 32 (60 mg, 0.15 mmol) in DMF (1 mL) and DIPEA (0.068 mL, 0.39 mmol), HATU (62.6 mg, 0.16 mmol) was added and the reaction mixture was stirred at rt for 3 h. The reaction was filtered and purified by preparative HPLC to afford the title compound (43 mg). LC-MS retention time=2.76 min; m/z=891.1 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Example 220

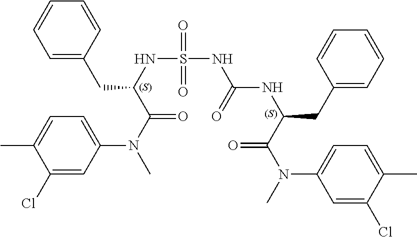

To a stirred solution of Intermediate BB-32.2 (150 mg, 0.49 mmol) in DCM (4 mL) was added chlorosulfonyl isocyanate (0.05 mL, 0.6 mmol), TEA (0.07 mL, 0.49 mmol) at 0° C. and the reaction mixture was stirred for 30 min. Then added 2,3-dihydro-1h-pyrrolo[2,3-b]pyridine (89 mg, 0.74 mmol) in DCM (10 mL) followed by TEA (0.07 mL, 0.49 mmol) and the reaction mixture was stirred at room temperature for 2 h. The reaction mixture was concentrated to dryness; the crude material was purified by preparative HPLC to afford the title compound (1.5 mg, an off white solid). Title compound: LC-MS retention time=1.48 min; m/z=708.1 [M−H]. Column: Acquity BEH C8 (2.1×50 mm) 1.7μ: Flow rate: 0.7 mL/min; Mobile Phase A: 5 mM NH₄OAc in water: ACN (95:5); Mobile Phase B: 5 mM NH₄OAc in water: ACN (5:95); 5% B to 95% B over 1.1 minutes and then hold a 0.6 min. at 95% B of flow rate 0.8 mL/min; Detection: UV at 220 nm. NMR (400 MHz, DMSO-d₆) δ ppm 9.71 (s, 1H), 7.84 (d, J=9.0 Hz, 1H), 7.35-7.29 (m, 2H), 7.25-7.17 (m, 6H), 7.07-6.83 (m, 8H), 6.58 (d, J=8.1 Hz, 1H), 4.37 (q, J=7.2 Hz, 1H), 4.09 (q, J=7.7 Hz, 1H), 3.06 (s, 3H), 3.00 (s, 3H), 2.86-2.77 (m, 2H), 2.69-2.58 (m, 2H), 2.33 (s, 3H), 2.30 (s, 3H).

### Example 221

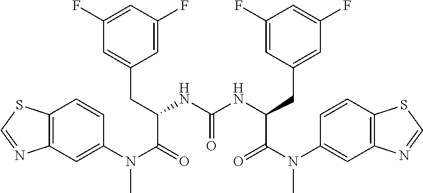 [SMILES: CN(C(=O)[C@H](Cc1cc(F)cc(F)c1)NC(=O)N[C@@H](Cc1cc(F)cc(F)c1)C(=O)N(C)c1ccc2scnc2c1)c1ccc2scnc2c1]

To a mixture of Intermediate 143 (0.060 g, 0.13 mmol) and 5,6-dihydro-7H-pyrrolo[2,3-c]pyridazine-7-sulfonamide (0.028 g, 0.14 mmol) in acetonitrile (2 mL) was added DBU (0.021 mL, 0.14 mmol). The reaction was stirred at ˜25° C. for 3.5 h. Another equivalent of DBU (0.021 mL, 0.14 mmol) was added and the reaction was heated at 40° C. for 3.5 h. The reaction was removed from the heat and all solvents were removed in vacuo. The residue was taken up in MeOH (2 mL) and purified via preparative LC/MS to afford the title compound (30.4 mg). LC-MS retention time=2.16 min; m/z=720.9 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Example 223

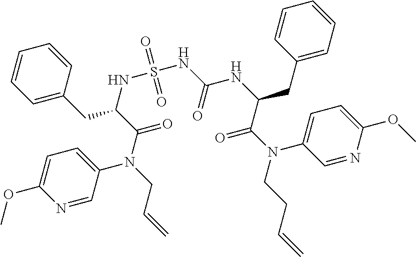

To a solution of Intermediate BB-34.1 (100 mg, 0.307 mmol) in DCM (12 mL) was added sulfurisocyanatidic chloride (0.030 mL, 0.34 mmol) at −15° C. and the reaction mixture was stirred for 1 h. The reaction mixture was added of hexane (20 mL) and the precipitated solid filtered and re-dissolved in dioxane (2 mL). To this solution was added a mixture of Intermediate BB-33.2 (96 mg, 0.307 mmol) and TEA (0.128 mL, 0.922 mmol) in dioxane (4 mL) at 5° C. and the resulting reaction mixture stirred at room temperature for 16 h. The reaction mixture was diluted with 10% aqueous NaHCO₃ solution (20 mL) and extracted with dichloromethane (3×20 mL). The combined organic layer was washed with water (20 mL), brine (20 mL), dried (Na₂SO₄), filtered, concentrated and the crude product was purified by preparative HPLC to afford the title product (2 mg) as an off white solid. LC-MS retention time=2.90 min; m/z=742.2 [M+H]⁺. Column: KINETIX XB-C18, 75×3 mm, 2.6 μm; Flow rate: 1 mL/min; Mobile Phase A: 10 mM HCOONH₄ in 98% Water/2% ACN; Mobile Phase B: 10 mM HCOONH₄ in 2% Water/98% ACN; 20% B to 100% B over 4 min, then hold for 0.6 min at 100% B with flow rate of 1.5 mL/min; Detection: UV at 220 nm.

### Example 224

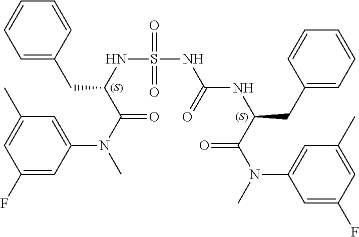

To a stirred solution of Intermediate 30.2 (101 mg, 0.35 mmol) in DCM (5 mL) was added chlorosulfonyl isocyanate (0.02 mL, 0.17 mmol), TEA (0.08 mL, 0.53 mmol) at 0° C. and the reaction mixture was stirred at room temperature for 12 h. The reaction mixture was concentrated to dryness and the crude product was purified by preparative HPLC to afford the title compound (59 mg) as an off white solid. LC-MS retention time=2.30 min; m/z=678.3 [M+H]⁺. Column: Ascentis Express C18 (50×2.1) mm, 2.7 μm; Flow: 1.1 mL/min; Mobile Phase A: 10 mM NH₄OAc in water: ACN (95:5); Mobile Phase B: 10 mM NH₄OAc in water: ACN (5:95); Temperature: 50° C.; 0% B to 100% B over 3 minutes; UV Detection at 220 nm.

### Example 225

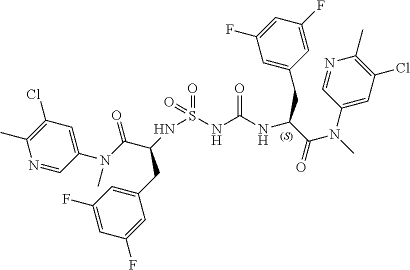

To a solution of sulfurisocyanatidic chloride (25 mg, 0.18 mmol) in DCM (2 mL) was added a solution of an HCl salt of Intermediate 146 (60 mg, 0.18 mmol) and TEA (0.04 mL, 0.3 mmol) in DCM (2 mL) in an ice-water bath and the reaction mixture was stirred for 20 min. A solution of 2,3-dihydro-1H-pyrrolo[2,3-b]pyridine (32 mg, 0.27 mmol) in DCM (2 mL) was added, followed by TEA (0.07 mL, 0.53 mmol), the reaction mixture was stirred for 2 min, the ice-water bath was removed and then stirred at rt for 2 h. The solvent was evaporated and the residue was redissolved in methanol and purified by preparative HPLC to afford the title compound (12.1 mg). LC-MS retention time=4.28 min; m/z=784.16 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Example 226

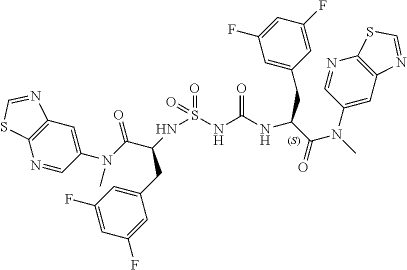

To a solution of sulfurisocyanatidic chloride (24 mg, 0.17 mmol) in DCM (2 mL) in an ice-water bath was added a solution of an HCl salt of Intermediate 149 (70 mg, 0.17 mmol) and TEA (0.07 mL, 0.5 mmol) in DCM (2 mL) and the reaction mixture was stirred for 2 min. Then a solution of 2,3-dihydro-1H-pyrrolo[2,3-b]pyridine (30 mg, 0.25 mmol) in DCM (2 mL) was added, followed by TEA (0.05 mL, 0.33 mmol), the ice-water bath was removed and the reaction mixture was stirred at rt for 2 h. The solvent was evaporated and the residue was redissolved in DMF and methanol and then purified by preparative HPLC to afford the title compound (5.8 mg). LC-MS retention time=3.75 min; m/z=802.09 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Example 228

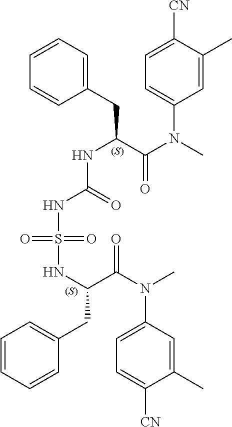

To a stirred solution of Intermediate BB-31.4 (0.100 g, 0.341 mmol) in DCM (5 mL) was added chlorosulfonyl isocyanate (0.015 mL, 0.170 mmol) and TEA (0.143 mL, 1.023 mmol) at 0° C. and the reaction mixture was allowed to stir at room temperature for 16 h. The reaction mixture was diluted with water (25 mL) and extracted with DCM (2×15 mL). The combined organic layer was washed with brine, dried (Na₂SO₄), filtered and concentrated. The crude product was purified by preparative HPLC purification to afford the title compound (26 mg) as an off white solid. LC-MS retention time=1.96 min; m/z=690.2 [M−H]. Column: Ascentis Express C18 (50×2.1) mm, 2.7 μm; Flow: 1.1 mL/min; Mobile Phase A: 10 mM NH₄OAc in water: ACN (95:5); Mobile Phase B: 10 mM NH₄OAc in water: ACN (5:95); Temperature: 50° C.; 0% B to 100% B over 3 minutes; UV Detection at 220 nm.

### Example 229

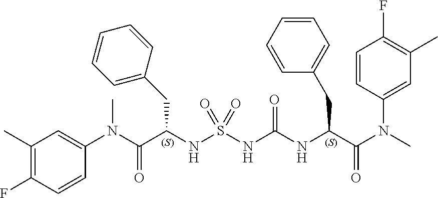

To a solution of (S)-2-amino-N-(4-fluoro-3-methylphenyl)-N-methyl-3-phenylpropanamide (0.1 g, 0.349 mmol) in DCM (1 mL) at 0° C. was added a solution of sulfurisocyanatidic chloride (0.015 mL, 0.175 mmol) in DCM (0.5 mL) followed by TEA (0.073 mL, 0.524 mmol) and the reaction mixture was stirred at room temperature for 4 h. The reaction mixture was concentrated to dryness and the crude material was purified by preparative HPLC to afford the title product (3 mg) as an off white solid. LCMS retention time=2.31 min; m/z=678.2 [M+H]⁺. Column: Ascentis Express C18 (50×2.1) mm, 2.7 μm; Flow: 1.1 mL/min; Mobile Phase A: 10 mM NH₄OAc in water: ACN (95:5); Mobile Phase B: 10 mM NH₄OAc in water: ACN (5:95); Temperature: 50° C.; 0% B to 100% B over 3 minutes; UV Detection at 220 nm. ¹H NMR (400 MHz, METHANOL-d₄) δ ppm 7.40-7.20 (m, 7H), 7.00-6.95 (m, 7H), 6.85-6.40 (m, 2H), 4.60-4.52 (m, 1H), 4.26 (dd, J=8.9, 6.0 Hz, 1H), 3.14 (s, 3H), 3.11 (s, 3H), 2.92-2.85 (m, 2H), 2.83-2.70 (m, 2H), 2.20 (S, 3H), 2.17 (S, 3H).

### Example 230

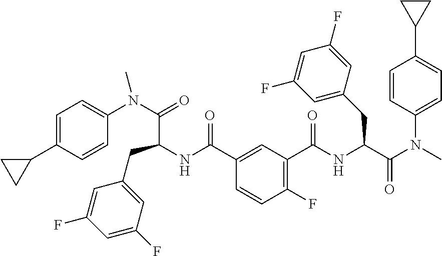 [SMILES: CN(C(=O)[C@H](Cc1cc(F)cc(F)c1)NC(=O)c1ccc(F)c(C(=O)N[C@@H](Cc2cc(F)cc(F)c2)C(=O)N(C)c2ccc(C3CC3)cc2)c1)c1ccc(C2CC2)cc1]

To a solution of an HCl salt of Intermediate 120 (55 mg, 0.14 mmol) and 4-fluoroisophthalic acid (12 mg, 0.06 mmol) in DMF (2 mL) was added DIPEA (0.07 mL, 0.4 mmol) and then HATU (50 mg, 0.13 mmol) and the reaction mixture was stirred at rt for 2 h. The solvent was evaporated and the residue was redissolved in DMF and methanol and then purified by preparative HPLC to afford the title compound (18.9 mg). LC-MS retention time=4.75 min; m/z=809.35 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Example 231

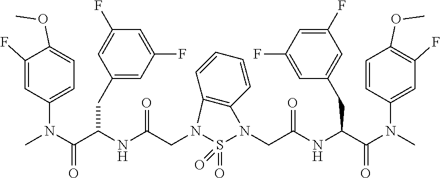 [SMILES: COc1ccc(N(C)C(=O)[C@H](Cc2cc(F)cc(F)c2)NC(=O)CN2c3ccccc3N(CC(=O)N[C@@H](Cc3cc(F)cc(F)c3)C(=O)N(C)c3ccc(OC)c(F)c3)S2(=O)=O)cc1F]

To a solution of an HCl salt of Intermediate 86 (28.3 mg, 0.073 mmol), Intermediate 51 (10 mg, 0.035 mmol) and DIPEA (0.049 mL, 0.28 mmol) in DMF (0.5 mL) was added HATU (28 mg, 0.073 mmol). The resulting mixture was stirred at rt ON and purified by preparative HPLC to afford the title compound (25.7 mg) as a white solid. LC-MS retention time=1.39 min; m/z=927.20 [M+H]+ (Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7 μm, Oven Temp.=40° C.).

### Example 232

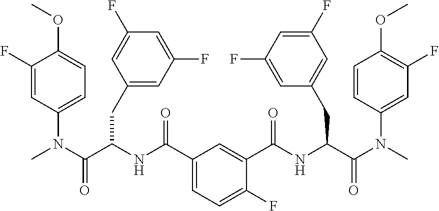 [SMILES: COc1ccc(N(C)C(=O)[C@H](Cc2cc(F)cc(F)c2)NC(=O)c2ccc(F)c(C(=O)N[C@@H](Cc3cc(F)cc(F)c3)C(=O)N(C)c3ccc(OC)c(F)c3)c2)cc1F]

To a solution of an HCl salt of Intermediate 86 (31 mg, 0.080 mmol), 4-fluoroisophthalic acid (7.0 mg, 0.038 mmol) and DIPEA (0.053 mL, 0.30 mmol) in DMF (0.5 mL) was added HATU (30 mg, 0.080 mmol). The resulting mixture was stirred at rt for 3 h and purified by preparative HPLC to afford the title compound (22 mg) as a white solid. LC-MS retention time=1.41 min; m/z=825.05 [M+H]+ (Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7 μm, Oven Temp.=40° C.).

### Example 233

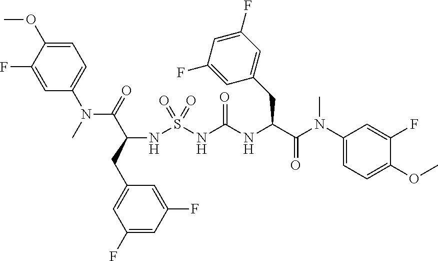

To a slurry of an HCl salt of Intermediate 86 (34 mg, 0.089 mmol) in DCM (1 mL) was added TEA (0.047 mL, 0.34 mmol). The resulting solution was then treated with sulfurisocyanatidic chloride (3.7 μL, 0.042 mmol) dropwise. The final solution was stirred at rt for 2 h. The solvent was evaporated and the residue was taken up into 0.5 mL DMF, filtered and purified by preparative HPLC to afford the title compound (12 mg) as a white solid. LC-MS retention time=1.35 min; m/z=782.05 [M+H]+ (Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7 μm, Oven Temp.=40° C.).

### Example 234

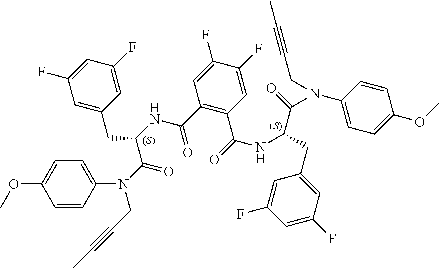

To a solution of an HCl salt of Intermediate 87 (395 mg, 0.999 mmol), 4,5-difluorophthalic acid (101 mg, 0.500 mmol) and HATU (399 mg, 1.05 mmol) in DMF (5 mL) was added DIPEA (0.698 mL, 4.00 mmol). The reaction mixture was stirred at rt for 3 h, diluted with EtOAc (50 mL), poured into water (50 mL), separated and the aqueous component was saturated with NaCl and extracted with EtOAc (20 mL). The combined organic components were washed with brine, dried over MgSO₄, filtered and evaporated in vacuo. The residue was taken up into DCM (5 mL) and purified by FCC (40 g silica gel cartridge), eluting with gradient 35%-65% EtOAc-hexanes to afford the title compound (325 mg) as a white powder. LC-MS retention time=1.55 min; m/z=883.25 [M+H]+ (Start % B=0, Final % B=98, Gradient Time=1.5 min, Flow Rate=0.8 ml/min, Wavelength=220, Solvent Pair=Water/Acetonitrile/TFA, Solvent A=100% Water/0.05% TFA, Solvent B=100% Acetonitrile/0.05% TFA, Column=Waters Aquity BEH C18 2.1×50 mm 1.7 μm, Oven Temp.=40° C.).

### Example 235

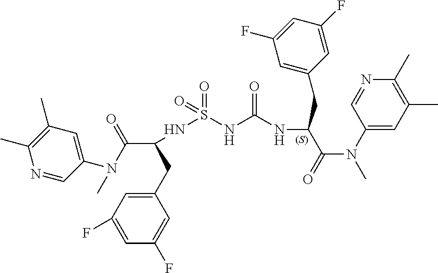

To a solution of sulfurisocyanatidic chloride (27 mg, 0.19 mmol) in DCM (2 mL) was added a solution of an HCl salt of Intermediate 152 (75 mg, 0.19 mmol) and DIPEA (0.07 mL, 0.38 mmol) in DCM (2 mL) in an ice-water bath and the reaction mixture was stirred for 2 min. Then a solution of 2,3-dihydro-1H-pyrrolo[2,3-b]pyridine (35 mg, 0.29 mmol) in DCM (2 mL) was added, followed by DIPEA (0.13 mL, 0.77 mmol). The reaction mixture was stirred for 2 min, the ice-water bath was removed and then it was stirred at rt for 1 h. The solvent was evaporated and the residue was redissolved in DMF and methanol and then purified by preparative HPLC to afford the title compound (24.2 mg). LC-MS retention time=3.17 min; m/z=744.25 [M+H]⁺. (Column: Phenomenex C18 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Example 236

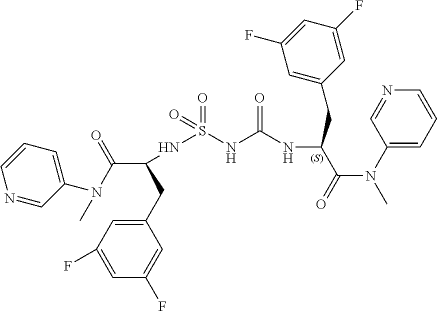

To a solution of sulfurisocyanatidic chloride (23 mg, 0.17 mmol) in DCM (1 mL) in an ice-water bath was added a solution of an HCl salt of Intermediate 154 (60 mg, 0.17 mmol) and DIPEA (0.06 mL, 0.3 mmol) in DCM (1 mL). Then a solution of 2,3-dihydro-1H-pyrrolo[2,3-b]pyridine (30 mg, 0.25 mmol) in DCM (1 mL) was added, followed by DIPEA (0.1 mL, 0.66 mmol). The reaction mixture was stirred for 2 min, the ice-water bath was removed and then it was stirred at rt for 2 h. The solvent was evaporated and the residue was redissolved in DMF and methanol and then purified by preparative HPLC to afford the title compound (3.4 mg). LC-MS retention time=3.17 min; m/z=688.10 [M+H]⁺. (Column: Phenomenex C18 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Example 237

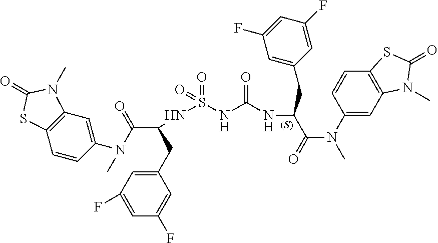

To a solution of sulfurisocyanatidic chloride (22 mg, 0.16 mmol) in DCM (2 mL) was added a solution of an HCl salt of Intermediate 157 (70 mg, 0.16 mmol) and DIPEA (0.05 mL, 0.3 mmol) in DCM (2 mL) in an ice-water bath and the reaction mixture was stirred for 2 min. Then a solution of 2,3-dihydro-1H-pyrrolo[2,3-b]pyridine (28.0 mg, 0.233 mmol) in DCM (2 mL) was added to the reaction mixture, followed by DIPEA (0.11 mL, 0.62 mmol). The reaction mixture was stirred for 2 min, the ice-water bath was removed and then it was stirred at rt for 2 h. The solvent was evaporated and the residue was redissolved in DMF and methanol and then purified by preparative HPLC to afford the title compound (7.6 mg). LC-MS retention time=4.01 min; m/z=860.20 [M+H]⁺. (Column: Phenomenex C18 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Example 238

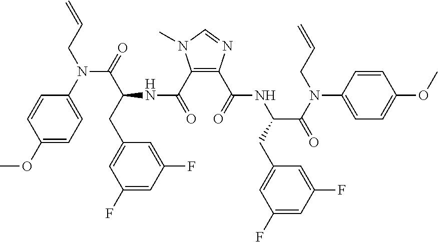

HATU (83 mg, 0.22 mmol) was added to a mixutre of 1-methyl-1H-imidazole-4,5-dicarboxylic acid (17.8 mg, 0.10 mmol) and an HCl salt of Intermediate 32 (80 mg, 0.21 mmol) in DMF (1 mL) and DIPEA (0.091 mL, 0.52 mmol) and the reaction mixture was stirred at rt for 16 h. The reaction was filtered and purified by preparative HPLC to afford the title compound (10.5 mg). LC-MS retention time=2.85 min; m/z=827.2 [M+H]⁺. (Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm).

### Example 239

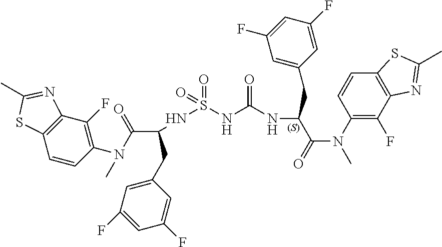

To a solution of sulfurisocyanatidic chloride (15 mg, 0.11 mmol) in DCM (1 mL) in an ice-water bath was added a solution of an HCl salt of Intermediate 162 (40 mg, 0.11 mmol) and TEA (0.018 mL, 0.11 mmol) in DCM (1 mL) and the reaction mixture was stirred for 1 min. Then a solution of 2,3-dihydro-1H-pyrrolo[2,3-b]pyridine (19 mg, 0.16 mmol) in DCM (1 mL) was added, followed by TEA (0.04 mL, 0.2 mmol), the reaction mixture was stirred for 2 min and then the ice-water bath was removed and it was then stirred at rt for 2 h. The reaction was concentrated and the residue was redissolved in DMF and methanol and then purified by preparative HPLC to afford the title compound (2.6 mg). LC-MS retention time=3.91 min; m/z=864.25 [M+H]⁺. (Column: Phenomenex C18 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

### Example 254

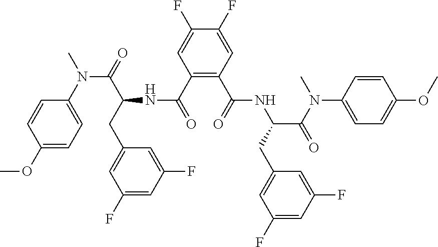

HATU (37.5 mg, 0.099 mmol) was added to a solution of an HCl salt of Intermediate 13 (32 mg, 0.090 mmol) and 4,5-difluorophthalic acid (9.1 mg, 0.045 mmol) in DMF (0.5 mL) and DIPEA (0.047 mL, 0.27 mmol) and the reaction mixture was stirred at rt ON. The reaction mixture was filtered and then purified by preparative HPLC to afford the title compound (28.0 mg). LC-MS retention time=2.30 min; m/z=807.2 [M+H]⁺. (Column: Waters BEH C18, 2.0×50 mm, 1.7-μm particles. Solvent A=95% Water: 5% MeOH: 10 mM NH₄OAc. Solvent B=5% Water: 95% MeOH: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 255

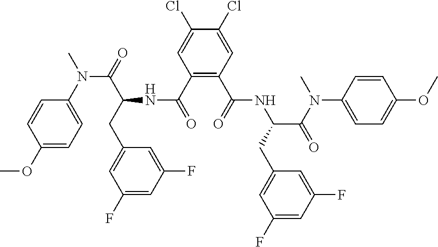

HATU (31.7 mg, 0.083 mmol) was added to a solution of an HCl salt of Intermediate 13 (27 mg, 0.076 mmol) and 4,5-dichlorophthalic acid (8.9 mg, 0.038 mmol) in DMF (0.5 mL) and DIPEA (0.040 mL, 0.23 mmol) and the reaction mixture was stirred at rt ON. The reaction mixture was filtered and then purified by preparative HPLC to afford the title compound (23.1 mg). LC-MS retention time=2.42 min; m/z=839.1 [M+H]⁺. (Column: Waters BEH C18, 2.0×50 mm, 1.7-μm particles. Solvent A=95% Water: 5% MeOH: 10 mM NH₄OAc. Solvent B=5% Water: 95% MeOH: 10 mM NH₄OAc. Flow Rate=0.5 mL/min. Start % B=0. Final % B=100. Gradient Time=3 minutes, then a 0.5-minute hold at 100% B. Wavelength=220 nm).

### Example 256

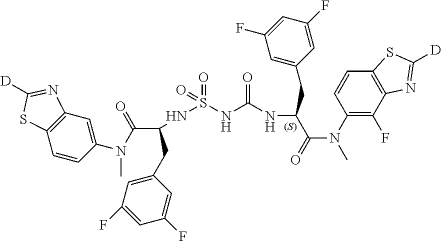

To a solution of sulfurisocyanatidic chloride (20.2 mg, 0.142 mmol) in DCM (1 mL) in an ice-water bath was added a solution of an HCl salt of Intermediate 95 (60 mg, 0.14 mmol) and TEA (0.05 mL, 0.3 mmol) in DCM (1 mL) and stirred for 1 min. Then a solution of 2,3-dihydro-1H-pyrrolo[2,3-b]pyridine (26 mg, 0.21 mmol) in DCM (1 mL) was added, followed by TEA (0.08 mL, 0.4 mmol), the ice-water bath was removed and the reaction mixture was stirred at rt for 3 h. The solvent was evaporated and the residue was redissolved in DMF and methanol, filtered and then purified by preparative HPLC to afford the title compound (2.9 mg). LC-MS retention time=3.98 min; m/z=802.28 [M+H]⁺. (Column: Phenomenex-Luna 2.0×50 mm, 3 μm particles; Mobile Phase A: 10% MeOH-90% H₂O-0.1% TFA; Mobile Phase B: 90% MeOH-10% H₂O-0.1% TFA; Temperature: 40° C.; Gradient: 0-100% B over 4 min, then a 1-min hold at 100% B; Flow: 0.8 mL/min; Detection: UV at 220 nm).

The foregoing description is merely illustrative and should not be understood to limit the scope or underlying principles of the invention in its various embodiments in any way. Indeed, various modifications of the invention, in addition to those shown and described herein, will become apparent to those skilled in the art from the foregoing description and examples. Such modifications are also intended to fall within the scope of the appended claims.

### Biological Methods

HIV cell culture assay—MT-2 cells, 293T cells and the proviral DNA clone of NL₄₋₃ virus were obtained from the NIH AIDS Research and Reference Reagent Program. MT-2 cells were propagated in RPMI 1640 media supplemented with 10% heat inactivated fetal bovine serum (FBS), 100 μg/ml penicillin G and up to 100 units/mL streptomycin. The 293T cells were propagated in DMEM media supplemented with 10% heat inactivated FBS, 100 μg/mL penicillin G and 100 μg/mL streptomycin. A recombinant NL₄₋₃ proviral clone, in which a section of the nef gene was replaced with the Renilla luciferase gene, was used to make the reference virus used in these studies. The recombinant virus was prepared through transfection of the recombinant NL₄₋₃ proviral clone into 293T cells using Transit-293 Transfection Reagent from Mirus Bio LLC (Madison, Wis.). Supernatent was harvested after 2-3 days and the amount of virus present was titered in MT-2 cells using luciferase enzyme activity as a marker by measuring luciferase enzyme activity. Luciferase was quantitated using the EnduRen Live Cell Substrate from Promega (Madison, Wis.). Antiviral activities of compounds toward the recombinant virus were quantified by measuring luciferase activity in MT-2 cells infected for 4-5 days with the recombinant virus in the presence of serial dilutions of the compound.

The 50% effective concentration (EC₅₀) was calculated by using the exponential form of the median effect equation where (Fa)=1/[1+(ED₅₀/drug conc.)^{m}] (Johnson V A, Byington R T. Infectivity Assay. In Techniques in HIV Research. ed. Aldovini A, Walker BD. 71-76. New York: Stockton Press.1990).

Compound cytotoxicity and the corresponding CC₅₀ values were determined using the same protocol as described in the antiviral assay except that uninfected cells were used. Cytotoxicity was assessed on day 4 in uninfected MT2 cells by using a XTT (2,3-bis[2-Methoxy-4-nitro-5-sulfophenyl]-2H-tetrazolium-5-carboxyanilide inner salt)-based colorimetric assay (Sigma-Aldrich, St Louis, Mo.).

Compounds demonstrate antiviral activity as depicted in Table 1 below. Unless specific values are provided, activity equal to A refers to a compound having an EC₅₀≤100 nM, while B and C denote compounds having an EC₅₀ between 100 nM and including 1 μM (B), or >1 μM (C).

| Example # | EC50 (μM) | CC50 (μM) |
|---|---|---|
| 1 | 2.86 | 23.1 |
| 2 | 0.03 | 77.9 |
| 3 | 1.94 | 70.9 |
| 4 | 1.87 | 100.0 |
| 5 | 0.15 | 33.3 |
| 6 | 6.78 | 100.0 |
| 7 | 3.76 | 100.0 |
| 8 | 2.04 | 33.3 |
| 9 | 2.67 | 100.0 |
| 10 | 4.71 | 10.5 |
| 11 | 0.47 | 33.3 |
| 12 | 12.37 | 29.0 |
| 13 | 0.08 | 100.0 |
| 14 | 0.08 | 50.0 |
| 15 | 4.22 | 35.3 |
| 16 | 1.72 | 33.3 |
| 17 | 12.25 | 15.9 |
| 18 | 0.03 | 85.3 |
| 19 | 0.17 | 99.5 |
| 20 | 0.01 | 35.0 |
| 21 | 3.25 | 27.0 |
| 22 | 1.31 | 21.1 |
| 23 | 1.16 | 26.9 |
| 24 | 0.09 | 4.2 |
| 25 | 1.13 | 3.7 |
| 26 | 0.90 | 31.1 |
| 27 | 0.04 | 100.0 |
| 28 | 1.40 | 30.0 |
| 29 | 2.49 | 31.8 |
| 30 | 4.96 | 31.2 |
| 31 | 0.78 | 3.5 |
| 32 | 3.29 | 34.6 |
| 33 | 1.24 | 100.0 |
| 34 | 0.01 | 100.0 |
| 35 | 0.03 | 99.9 |
| 36 | 7.27 | 65.3 |
| 37 | 0.07 | 19.9 |
| 38 | 0.55 | 45.0 |
| 39 | 0.95 | 33.3 |
| 40 | 0.31 | 36.0 |
| 41 | 0.10 | 23.0 |
| 42 | 0.85 | 40.7 |
| 43 | 0.48 | 3.7 |
| 44 | 0.02 | 67.0 |
| 45 | 0.01 | 38.8 |
| 46 | 0.27 | 18.5 |
| 47 | 2.76 | 11.1 |
| 48 | 0.17 | 30.9 |
| 49 | 0.04 | 10.8 |
| 50 | 0.04 | 3.7 |
| 51 | 0.01 | 100.0 |
| 52 | 1.14 | 22.6 |
| 53 | 0.34 | 33.3 |
| 55 | 10.06 | 85.8 |
| 56 | 2.60 | 10.5 |
| 57 | 1.58 | 3.5 |
| 58 | 0.61 | 3.3 |
| 59 | 3.34 | 11.0 |
| 61 | 0.61 | 32.6 |
| 62 | 0.49 | 100.0 |
| 63 | 1.09 | 30.2 |
| 64 | 0.38 | 11.3 |
| 65 | 0.32 | 34.6 |
| 68 | 1.42 | 69.3 |
| 69 | 0.02 | 11.8 |
| 70 | 0.15 | 100.0 |
| 71 | 0.08 | 6.5 |
| 72 | 0.04 | 4.4 |
| 73 | 0.07 | 39.9 |
| 74 | 0.03 | 11.3 |
| 75 | 0.08 | 11.5 |
| 76 | 0.03 | 11.4 |
| 77 | 3.98 | 9.6 |
| 78 | 50.00 | 50.0 |
| 79 | 1.40 | 100.0 |
| 80 | 0.16 | 12.8 |
| 81 | 0.00 | 2.8 |
| 82 | 0.07 | 100.0 |
| 83 | 0.09 | 6.2 |
| 84 | 0.04 | 6.9 |
| 85 | 0.97 | 100.0 |
| 86 | 0.09 | 24.5 |
| 87 | 0.09 | 11.1 |
| 88 | 13.68 | 20.8 |
| 90 | 0.34 | 22.7 |
| 92 | 0.29 | 10.1 |
| 93 | 0.09 | 5.7 |
| 94 | 0.16 | 23.2 |
| 95 | 0.21 | 50.0 |
| 96 | 0.12 | 50.0 |
| 97 | 2.60 | 100.0 |
| 98 | 0.03 | 11.2 |
| 99 | 0.02 | 1.8 |
| 100 | 0.07 | 100.0 |
| 101 | 0.03 | 100.0 |
| 102 | 0.05 | 33.3 |
| 103 | 0.15 | 33.3 |
| 104 | 0.13 | 31.0 |
| 105 | 0.68 | 3.1 |
| 106 | 0.05 | 6.3 |
| 107 | 0.30 | 100.0 |
| 108 | 0.72 | 100.0 |
| 109 | 6.87 | 65.4 |
| 110 | 0.21 | 100.0 |
| 111 | 0.96 | 100.0 |
| 112 | 1.71 | 1.3 |
| 113 | 0.36 | 25.3 |
| 114 | 1.78 | 27.1 |
| 115 | 2.13 | 100.0 |
| 116 | 0.71 | 1.5 |
| 117 | 0.64 | 30.8 |
| 118 | 0.00 | 100.0 |
| 119 | 0.01 | 100.0 |
| 120 | 0.26 | 23.3 |
| 121 | 0.73 | 18.8 |
| 122 | 0.09 | 100.0 |
| 123 | 0.38 | 19.2 |
| 124 | 3.80 | 31.0 |
| 125 | 0.34 | 100.0 |
| 126 | 0.01 | 100.0 |
| 127 | 0.01 | 3.8 |
| 128 | 0.08 | 12.6 |
| 129 | 0.01 | 5.3 |
| 131 | 0.49 | 6.3 |
| 132 | 1.94 | 7.3 |
| 133 | 0.41 | 13.9 |
| 134 | 0.82 | 10.8 |
| 135 | 0.09 | 0.5 |
| 136 | 0.34 | 100.0 |
| 138 | 0.26 | 12.9 |
| 139 | 0.04 | 33.3 |
| 140 | 0.10 | 32.7 |
| 141 | 0.09 | 16.2 |
| 142 | 0.82 | 17.4 |
| 145 | 12.04 | 19.1 |
| 146 | 2.86 | 30.6 |
| 147 | 0.09 | 100.0 |
| 148 | 0.72 | 100.0 |
| 149 | 1.64 | 79.5 |
| 150 | 0.01 | 24.6 |
| 151 | 0.19 | 33.4 |
| 152 | 0.06 | 99.3 |
| 153 | 1.54 | 3.1 |
| 154 | 1.86 | 100.0 |
| 155 | 2.13 | 33.3 |
| 156 | 24.73 | 50.0 |
| 158 | 9.53 | 12.4 |
| 159 | 3.68 | 86.4 |
| 160 | 0.02 | 100.0 |
| 161 | 2.80 | 73.9 |
| 162 | 0.07 | 43.9 |
| 163 | 2.56 | 100.0 |
| 164 | 2.64 | 100.0 |
| 165 | 0.04 | 30.3 |
| 166 | 6.70 | 100.0 |
| 167 | 0.27 | 26.5 |
| 168 | 0.15 | 16.2 |
| 169 | 0.06 | 4.5 |
| 170 | 7.66 | 28.4 |
| 171 | 100.00 | 100.0 |
| 172 | 0.58 | 100.0 |
| 173 | 0.00 | 25.5 |
| 174 | 100.00 | 100.0 |
| 175 | 0.45 | 100.0 |
| 176 | 0.04 | 100.0 |
| 177 | 0.27 | 88.0 |
| 178 | 0.02 | 100.0 |
| 179 | 0.34 | 100.0 |
| 180 | 0.03 | 80.1 |
| 181 | 0.02 | 85.8 |
| 182 | 0.40 | 97.9 |
| 182B | 0.02 | 13.4 |
| 183 | 0.09 | 78.9 |
| 184 | 0.19 | 11.1 |
| 185 | 0.08 | 96.0 |
| 186 | 6.15 | 100.0 |
| 187 | 3.78 | 100.0 |
| 188 | 0.18 | 100.0 |
| 189 | 0.01 | 8.2 |
| 190 | 0.77 | 1.6 |
| 191 | 1.79 | 100.0 |
| 192 | 7.24 | 58.2 |
| 193 | 1.74 | 33.3 |
| 194 | 0.58 | 41.9 |
| 195 | 0.02 | 54.0 |
| 196 | 0.37 | 100.0 |
| 197 | 0.01 | 9.3 |
| 198 | 0.01 | 12.5 |
| 199 | 0.47 | 46.7 |
| 200 | 0.39 | 100.0 |
| 201 | 0.48 | 12.5 |
| 202 | 0.08 | 100.0 |
| 203 | 30.38 | 100.0 |
| 204 | 14.97 | 33.3 |
| 205 | 0.18 | 38.7 |
| 206 | 14.45 | 33.3 |
| 207 | 25.00 | 25.0 |
| 208 | 16.03 | 37.7 |
| 209 | 0.42 | 100.0 |
| 210 | 10.14 | 100.0 |
| 211 | 3.92 | 14.0 |
| 212 | 3.73 | 100.0 |
| 213 | 8.21 | 100.0 |
| 214 | 0.01 | 43.8 |
| 215 | 0.31 | 34.5 |
| 216 | | |
| 217 | | |
| 218 | | |
| 219 | | |
| 220 | 3.80 | 16.7 |
| 221 | 0.52 | 100.0 |
| 223 | 1.50 | 25.0 |
| 224 | 3.91 | 46.0 |
| 225 | 0.87 | 2.3 |
| 226 | 8.92 | 100.0 |
| 228 | 2.74 | 93.5 |
| 229 | 2.64 | 64.7 |
| 230 | 0.02 | 100.0 |
| 231 | 0.04 | 100.0 |
| 232 | 0.18 | 33.3 |
| 233 | 0.03 | 35.8 |
| 234 | 6.83 | 95.3 |
| 235 | 7.00 | 100.0 |
| 236 | 4.14 | 100.0 |
| 237 | 0.27 | 71.9 |
| 238 | | |
| 239 | 0.86 | 100.0 |
| 254 | 0.09 | 10.0 |
| 255 | 0.08 | 10.0 |
| 256 | 0.02 | 58.7 |

The foregoing description is merely illustrative and should not be understood to limit the scope or underlying principles of the invention in its various embodiments in any way. Indeed, various modifications of the invention, in addition to those shown and described herein, will become apparent to those skilled in the art from the foregoing description and examples. Such modifications are also intended to fall within the scope of the appended claims.

What is claimed is:

1. A compound of Formula I, including pharmaceutically acceptable salts thereof: 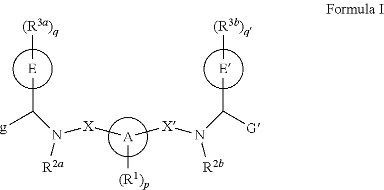
wherein:
A is a bond or is selected from C₁-C₅ alkyl, C₂-C₅ alkenyl, C₂-C₅ alkynyl, aryl, C₃-C₆ cycloalkyl, C₂-C₅ bicycloalkyl, —CO—, —CS—, —C(═N—CN)—, heterocyclyl, nitrogen, sulfur, oxygen, —O—(C₂-C₄ alkyl)-O—, —N(R^{xa})CON(R^{xb})—, and ferrocene;
each R¹ is independently selected from hydrogen, C₁-C₄ alkyl, C₂-C₄ alkenyl, C₁-C₄ alkoxy, C₂-C₄ (alkoxyalkyl), (C₁-C₄ alkoxy)carbonyl, C₁-C₄ alkylthioxy, benzyloxy, C₂-C₄ alkynyl, aryl, carboxylic acid, cyano, halogen, C₁-C₄ haloalkyl, C₁-C₄ haloalkoxy, heterocyclyl, hydroxy, C₁-C₄ hydroxyalkyl, thioxy, —CH₂NH₂, —(C₁-C₄ alkyl)-heteroaryl, —CO—(C₁-C₄ alkyl), —CO(R^{y}), —CON(R^{xa})₂, —NHCON(R^{xa})₂, —NHCO—(C₁-C₄ alkyl), —NHCO₂—(C₁-C₄ alkyl), —NHSO₂—(C₁-C₄ alkyl), —OCH₂-aryl, —SO₂—(C₁-C₄ alkyl), —SO₂—N(R^{xa})₂, —SO₂-heterocyclyl, —N(R^{xa})₂, and nitro;
p is from 0 to 5;
R^{xa} and R^{xb} are independently selected from hydrogen, alkyl, or haloalkyl;
R^{y} is selected from C₂-C₄ (dialkylamine) or nitrogen-containing heterocyclyl, and is attached to the parent fragment through its nitrogen;
X and X¹ are each are independently a bond or are selected from: 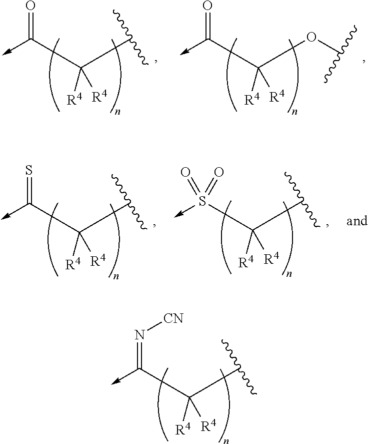
wherein the attachment of X and X¹ to the parent structure is such that the bond with the arrow is oriented toward the respective nitrogen shown in Formula I; provided, however, that when A is a bond, at least one X or X¹ is not a bond;
each n is independently from 0 to 2;
each R⁴ is independently selected from hydrogen, C₁-C₃ alkyl, C₁-C₃ alkenyl, aryl, aryl(C₁-C₂ alkyl), hydroxyl, and halogen, with the option for two R⁴ on the same or adjacent carbon(s) to form a ring;
R^{2a} and R^{2b} are independently selected from hydrogen, C₁-C₄ alkyl, C₃-C₄ alkenyl, C₃-C₅ alkynyl and C₃-C₄ cycloalkyl, and each is optionally substituted with 1 to 3 substituents selected from halogen, hydroxyl, C₁-C₂ alkoxy, and C₁-C₂ haloaloxy;
G and G′ are each independently selected from; 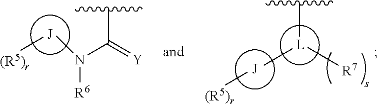
each Y is independently oxygen or sulfur;
each J is a bond or is independently selected from aryl, heterocyclyl, or C₃-C₇ cycloalkyl;
each R⁵ is independently selected from hydrogen, C₁-C₄ alkoxy, C₁-C₄ alkyl, halogen, C₂-C₅ bicycloalkyl, C₁-C₄ haloalkoxy, C₁-C₄ haloalkyl, —CONH₂, —CN, —NHCO(C₁-C₄ alkyl), —NHCON(C₁-C₄ alkyl)₂, —NHCO₂(C₁-C₄ alkyl), —OH, —SO₂N(C₁-C₄ alkyl)₂ and heterocyclyl;
each r is independently from 0 to 5;
each R⁶ is independently selected from hydrogen, C₁-C₄ alkyl, C₂-C₄ alkenyl, and C₃-C₄ cycloalkyl, optionally substituted with halogen, hydroxyl, C₁-C₂ alkoxy, or C₁-C₂ haloalkoxy;
each L is independently selected from a five or six-member heteroaryl ring;
each R⁷ is independently selected from C₁-C₃ alkoxy, C₁-C₃ alkyl, halogen, C₁-C₃ haloalkoxy, C₁-C₃ haloalkyl, —CONH₂, —CN, —OH, —C₂-C₅ alkynol, —NHCO(C₁-C₃ alkyl), —NHCON(C₁-C₃ alkyl)₂, —NHCO₂(C₁-C₃ alkyl), —SO₂N(C₁-C₃ alkyl)₂, and C₂-C₆ alkyne optionally substituted with 1 to 2 halides;
each s is independently from 0 to 4;
E and E′ are each independently selected from C₁-C₈ alkyl, C₂-C₈ alkenyl, C₂-C₈ alkynyl, C₅-C₈ bicycloalkyl, C₃-C₇ cycloalkyl, aryl, heterocyclyl, and a C₁-C₂ alkyl group containing any one of the following groups: C₅-C₈ bicycloalkyl, C₃-C₇ cycloalkyl, aryl, and heterocyclyl;
R^{3a} and R^{3b} are each independently selected from C₂-C₄ alkenoxy, C₂-C₄ alkenyl, C₁-C₄ alkoxy, C₂-C₄ (alkoxyalkyl), (C₁-C₄ alkoxy)carbonyl, C₁-C₄ alkyl, C₁-C₄ haloalkyl, C₁-C₄ haloalkoxy, carboxyamide, halogen, —CN, —NHCO(C₁-C₄ alkyl), —OH, C₁-C₄ hydroxyalkyl, and —SO₂N-heterocycle; and
q and q′ are each independently from 0 to 5;
wherein the attachment of each of “X”, “X¹” or N to “A” could be on the same or different atom(s) of “A”.

2. The compound of claim 1 wherein A is a bond.

3. The compound of claim 2 wherein at least one of X and X¹ are independently selected from:

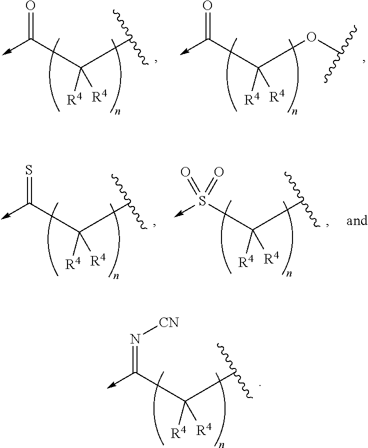

4. The compound of claim 1 wherein A is selected from C₁-C₅ alkyl, C₂-C₅ alkenyl, C₂-C₅ alkynyl, aryl, C₃-C₆ cycloalkyl, C₂-C₅ bicycloalkyl, —CO—, —CS—, —C(═N—CN)—, heterocyclyl, nitrogen, sulfur, oxygen, —O—(C₂-C₄ alkyl)-O—, —N(R^{xa})CON(R^{xb})—, and ferrocene.

5. The compound of claim 4 wherein at least one of X and X¹ are a bond.

6. A compound of Formula II, including pharmaceutically acceptable salts thereof: 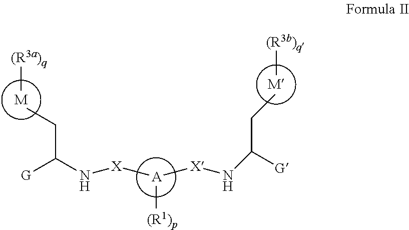
wherein
A is a bond or is selected from C₁-C₅ alkyl, C₂-C₅ alkenyl, C₂-C₅ alkynyl, aryl, C₃-C₆ cycloalkyl, C₂-C₅ bicycloalkyl, —CO—, —CS—, —C(═N—CN)—, heterocyclyl, nitrogen, sulfur, oxygen, —O—(C₂-C₄ alkyl)-O—, —N(R^{xa})CON(R^{xb})—, and ferrocene;
each R¹ is independently selected from hydrogen, C₁-C₄ alkyl, C₂-C₄ alkenyl, C₁-C₄ alkoxy, C₂-C₄ (alkoxyalkyl), (C₁-C₄ alkoxy)carbonyl, C₁-C₄ alkylthioxy, benzyloxy, C₂-C₄ alkynyl, aryl, carboxylic acid, cyano, halogen, C₁-C₄ haloalkyl, C₁-C₄ haloalkoxy, heterocyclyl, hydroxy, C₁-C₄ hydroxyalkyl, thioxy, —CH₂NH₂, —(C₁-C₄ alkyl)-heteroaryl, —CO—(C₁-C₄ alkyl), —CO(R^{y}), —CON(R^{xa})₂, —NHCON(R^{xa})₂, —NHCO—(C₁-C₄ alkyl), —NHCO₂—(C₁-C₄ alkyl), —NHSO₂—(C₁-C₄ alkyl), —OCH₂-aryl, —SO₂—(C₁-C₄ alkyl), —SO₂—N(R^{xa})₂, —SO₂-heterocyclyl, —N(R^{xa})₂, and nitro;
p is from 0 to 5;
R^{xa} and R^{xb} are independently selected from hydrogen, alkyl, or haloalkyl;
R^{y} is selected from C₁-C₂ dialkylamine or nitrogen-containing heterocyclyl, and is attached to the parent fragment through its nitrogen;
X and X¹ are each are independently a bond or are selected from: 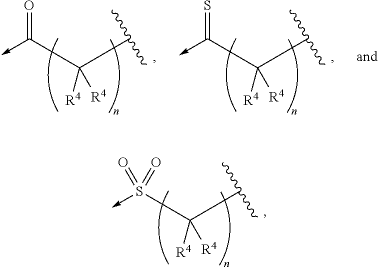
wherein the attachment of X, X¹ to the parent structure is such that the bond with the arrow is oriented toward the respective nitrogen shown in Formula II; provided, however, that when A is a bond, at least one X or X¹ is not a bond;
each n is independently from 0 to 2;
each R⁴ is independently selected from hydrogen, C₁-C₃ alkyl, C₂-C₃ alkenyl, aryl, aryl(C₁-C₂ alkyl), hydroxyl, halogen with the option for two R⁴s on same or adjacent carbon(s) to form a ring;
G and G′ are each independently selected from 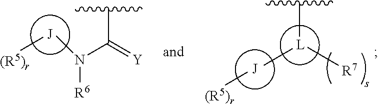
each Y is independently oxygen or sulfur;
each J is independently a bond or selected from aryl, heterocyclyl, or C₃-C₇ cycloalkyl;
each R⁵ is independently selected from hydrogen, C₁-C₄ alkoxy, C₂-C₄ (alkoxyalkyl), C₁-C₄ alkyl, halogen, C₂-C₅ bicycloalkyl, C₁-C₄ haloalkoxy, C₁-C₄ haloalkyl, —CONH₂, —CN, —NHCO(C₁-C₄ alkyl), —NHCON(C₁-C₄ alkyl)₂, —NHCO₂(C₁-C₄ alkyl), —OH, —SO₂N(C₁-C₄ alkyl)₂ and heterocyclyl;
each r is independently from 0 to 5;
each R⁶ is independently selected from hydrogen, C₁-C₄ alkyl, C₂-C₄ alkenyl, and C₃-C₄ cycloalkyl, optionally substituted with halogen, hydroxyl, C₁-C₂ alkoxy, or C₁-C₂ haloalkoxy;
each L is independently selected from a five or six-member heteroaryl ring;
each R⁷ is independently selected from C₁-C₃ alkoxy, C₁-C₃ alkyl, halogen, C₁-C₃ haloalkoxy, C₁-C₃ haloalkyl, —CONH₂, —CN, —OH, —C₂-C₅ alkynol, —NHCO(C₁-C₃alkyl), —NHCON(C₁-C₃alkyl)₂, —NHCO₂(C₁-C₃ alkyl), and —SO₂N(C₁-C₃alkyl)₂, and C₂-C₆ alkyne optionally substituted with 1 to 2 halides;
each s is independently from 0 to 4;
M and M′ are independently selected from C₁-C₇ alkyl, C₂-C₇ alkenyl, C₂-C₇ alkynyl, C₅-C₈ bicycloalkyl, C₃-C₇ cycloalkyl, aryl, and heterocyclyl;
each R^{3a} and R^{3b} is independently selected from C₂-C₄ alkenoxy, C₂-C₄ alkenyl, C₁-C₄ alkoxy, C₂-C₄ (alkoxyalkyl), (C₁-C₄alkoxy)carbonyl, C₁-C₄ alkyl, C₁-C₄ haloalkyl, C₁-C₄ haloalkoxy, carboxyamide, halogen, —CN, —NHCO(C₁-C₄ alkyl), —OH, C₁-C₄ hydroxyalkyl, and —SO₂N-heterocycle; and
q and q′ are each independently from 0 to 5;
wherein the attachment of “X”, “X¹” or N to “A” could be on the same or different atom(s) of “A”.

7. A compound of Formula III, including pharmaceutically acceptable salts thereof: 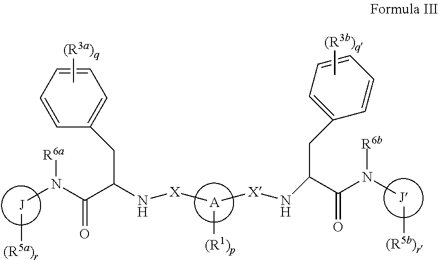
wherein
A is a bond or is selected from C₁-C₅ alkyl, C₂-C₅ alkenyl, C₂-C₅ alkynyl, aryl, C₃-C₆ cycloalkyl, C₂-C₅ bicycloalkyl, —CO—, —CS—, —C(═N—CN)—, heterocyclyl, nitrogen, sulfur, oxygen, —O—(C₂-C₄ alkyl)-O—, —N(R^{xa})CON(R^{xb})—, and ferrocene;
each R¹ is independently selected from hydrogen, C₁-C₄ alkyl, C₂-C₄ alkenyl, C₁-C₄ alkoxy, C₂-C₄ (alkoxyalkyl), (C₁-C₄ alkoxy)carbonyl, C₁-C₄ alkylthioxy, benzyloxy, C₂-C₄ alkynyl, aryl, carboxylic acid, cyano, halogen, C₁-C₄ haloalkyl, C₁-C₄ haloalkoxy, heterocyclyl, hydroxy, C₁-C₄ hydroxyalkyl, thioxy, —CH₂NH₂, —(C₁-C₄ alkyl)-heteroaryl, —CO—(C₁-C₄ alkyl), —CO(R^{y}), —CON(R^{xa})₂, —NHCON(R^{xa})₂, —NHCO—(C₁-C₄ alkyl), —NHCO₂—(C₁-C₄ alkyl), —NHSO₂—(C₁-C₄ alkyl), —OCH₂-aryl, —SO₂—(C₁-C₄ alkyl), —SO₂—N(R^{xa})₂, —SO₂-heterocyclyl, —N(R^{xa})₂, and nitro;
p is from 0 to 5;
R^{xa} and R^{xb} are independently selected from hydrogen, alkyl, or haloalkyl;
R^{y} is selected from C₁-C₂ dialkylamine or a nitrogen-containing heterocyclyl, and is attached to the parent fragment through its nitrogen;
X and X¹ are each are independently a bond or are selected from: 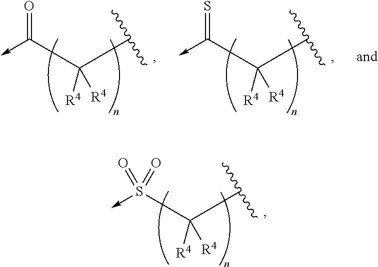
wherein the attachment of X, X¹ to the parent structure is such that the bond with the arrow is oriented toward the respective nitrogen shown in Formula III; provided, however, that when A is a bond, at least one X or X¹ is not a bond;
each n is independently from 0 to 2;
each R⁴ is independently selected from C₁-C₃ alkyl, C₂-C₃ alkenyl, aryl, aryl(C₁-C₂ alkyl), hydroxyl, and halogen, with the option for two R⁴ on the same or adjacent carbon(s) to form a ring;
J and J′ are independently a bond or are independently selected from aryl, heterocyclyl, or C₃-C₇ cycloalkyl;
R^{5a} and R^{5b} are independently selected from hydrogen, C₁-C₄ alkoxy, C₁-C₄ alkyl, C₂-C₄ (alkoxyalkyl), C₃-C₄ cycloalkyl, halogen, C₁-C₄ haloalkoxy, C₁-C₄ haloalkyl, —CONH₂, —CN, —NHCO(C₁-C₄ alkyl), —NHCON(C₁-C₄ alkyl)₂, —NHCO₂(C₁-C₄ alkyl), —OH, —SO₂N(C₁-C₄ alkyl)₂ and heterocyclyl;
each r and r′ is independently from 0 to 4;
R^{6a} and R^{6b} are each independently selected from hydrogen, C₁-C₄ alkyl, C₂-C₄ alkenyl, and C₃-C₄ cycloalkyl, optionally substituted with halogen, hydroxyl, C₁-C₂ alkoxy, or C₁-C₂ haloalkoxy;
each R^{3a} and R^{3b} is independently selected from C₂-C₄ alkenoxy, C₂-C₄ alkenyl, C₁-C₄ alkoxy, C₂-C₄ (alkoxyalkyl), (C₁-C₄ alkoxy)carbonyl, C₁-C₄ alkyl, C₁-C₄ haloalkyl, C₁-C₄ haloalkoxy, carboxyamide, halogen, —CN, —NHCO(C₁-C₄ alkyl), —OH, C₁-C₄ hydroxyalkyl, and —SO₂N-heterocycle; and
q and q′ are independently from 0 to 4;
wherein the attachment of “X”, “X¹” or N to “A” could be on the same or different atom(s) of “A”.

8. The compound as claimed in claim 7, wherein A is selected from C₁-C₅ alkyl, C₂-C₅ alkenyl, aryl with 1 to 2 rings, C₃-C₆ cycloalkyl, —CO—, heterocyclyl with 1 to 2 rings, nitrogen, sulfur, oxygen, —O—(C₂-C₄ alkyl)-O—, —N(R^{xa})CON(R^{xb})—, and ferrocene;
each R¹ is independently selected from hydrogen, C₁-C₄ alkyl, C₂-C₃ alkenyl, C₁-C₂ alkoxy, aryl, carboxylic acid, cyano, halogen, C₁-C₂ haloalkyl, C₁-C₂ haloalkoxy, heterocyclyl, hydroxy, C₁-C₄ hydroxyalkyl, —CO—(C₁-C₄ alkyl), CO(R^{y}), —CON(R^{xa})₂, —NHCON(R^{xa})₂, —SO₂—(C₁-C₄ alkyl), —SO₂—N(R^{xa})₂, —SO₂-heterocyclyl, and —N(R^{xa})₂;
p is from 0 to 4;
each R⁴ is independently selected from hydrogen, C₁-C₃ alkyl, aryl(C₁-C₂ alkyl), hydroxyl, or halogen with the option for two “R⁴”s on the same or adjacent carbon(s) to form a ring; and
n is from 0 to 2.

9. The compound as claimed in claim 8, wherein A is selected from C₁-C₅ alkyl, C₂-C₅ alkenyl, aryl with 1 to 2 rings, C₃-C₆ cycloalkyl, —CO—, heterocyclyl with 1 to 2 rings, nitrogen, oxygen, —O—(C₂-C₄ alkyl)-O—, —N(R^{xa})CON(R^{xb})—, and ferrocene;
each R¹ is independently selected from the group of hydrogen, C₁-C₄ alkyl, C₂-C₃ alkenyl, C₁-C₂ alkoxy, aryl, carboxylic acid, cyano, halogen, C₁-C₂ haloalkyl, C₁-C₂ haloalkoxy, heterocyclyl, hydroxy, C₁-C₄ hydroxyalkyl, —CO—(C₁-C₄ alkyl), CO(R^{y}), —CON(R^{xa})₂, —NHCON(R^{xa})₂, —SO₂—(C₁-C₄ alkyl), —SO₂—N(R^{xa})₂, —SO₂-heterocyclyl, and —N(R^{xa})₂;
p is from 0 to 4;
each R⁴ is independently selected from hydrogen, C₁-C₃ alkyl, aryl(C₁-C₂ alkyl), hydroxyl, or halogen with the option for two “R⁴”s on the same or adjacent carbon(s) to form a ring; and
n is from 0 to 2.

10. The compound as claimed in claim 8, wherein each R^{3a} and R^{3b} is independently selected from C₂-C₄ alkenoxy, C₂-C₄ alkenyl, C₁-C₄ alkoxy, C₁-C₄ alkyl, C₁-C₄ haloalkyl, C₁-C₄ haloalkoxy, halogen, —CN, and —OH;
q and q′ are independently from 0 to 3;
J and J′ are independently selected from 1-2 ring aryl, and 1-2 ring heteroaryl;
R^{5a} and R^{5b} are independently selected from hydrogen, C₁-C₄ alkoxy, C₁-C₄ alkyl, C₃-C₄ cycloalkyl, halogen, C₁-C₄ haloalkoxy, C₁-C₄ haloalkyl, C₃-C₄ cycloalkyl, —CONH₂, —CN, —NHCO(C₁-C₂ alkyl), —NHCON(C₁-C₂alkyl)₂, —NHCO₂(C₁-C₂ alkyl), —OH, and heterocyclyl;
r and r′ are independently from 0 to 4; and
R^{6a} and R^{6b} are independently selected from hydrogen, C₁-C₄ alkyl, C₁-C₄ alkenyl, or C₃-C₄ cycloalkyl, and with the option for each to be substituted with halogen.

11. The compound as claimed in claim 10, wherein each R^{3a} and R^{3b} is independently selected from C₂-C₄ alkenyl, C₁-C₂ alkoxy, C₁-C₄ alkyl, C₁-C₃ haloalkyl, C₁-C₃ haloalkoxy, halogen, and —CN;
q and q′ are independently from 0 to 3;
J and J′ are independently selected from 1-2 ring aryl, and 1-2 ring heteroaryl;
R^{5a} and R^{5b} are independently selected from the group of C₁-C₄ alkoxy, C₁-C₄ alkyl, C₃-C₄ cycloalkyl, halogen, C₁-C₄ haloalkoxy, C₁-C₄ haloalkyl, C₃-C₄ cycloalkyl, —CONH₂, —CN, —NHCO(C₁-C₂ alkyl), —NHCON(C₁-C₂alkyl)₂, —NHCO₂(C₁-C₂ alkyl), —OH, and heterocyclyl;
r and r′ are independently from 0 to 4; and
R^{6a} and R^{6b} are independently selected from hydrogen, C₁-C₄ alkyl, C₁-C₄ alkenyl, or C₃-C₄ cycloalkyl, and with the option for each to be substituted with halogen.

12. The compound as claimed in claim 8, wherein A is selected from CO, nitrogen, sulfur, oxygen, (CH₂)ₜ where t=1-4, —CH═CH—, —CH═C(Me)CH₂—, —CH═CH—CH₂—, —OCH₂CH₂O—, —NH(CO)NH—, cyclopentyl, cyclohexyl, phenyl, biphenyl, pyridine, pyrimidine, bipyrimidine, pyridazine, pyrazine, triazine, piperizine, pyrazole, thiophene, imidazole, isoxazole, indole, 1,3-dihydrobenzo[c][1,2,5]thiadiazole 2,2-dioxide, 1H-benzo[d]imidazol-2(3H)-one, imidazolidin-2-one, 2,3-dihydrophthalazine-1,4-dione, quinoxaline-2,3(1H,4H)-dione, 3-hydroxyquinoxalin-2(1H)-one, quinazoline-2,4(1H,3H)-dione, and ferrocene;
each R¹ is independently selected from H, C₁-C₄ alkyl, C₁-C₂ haloalkyl, C₁-C₂ alkoxy, C₁-C₂ haloalkoxy, C₁-C₄ hydroxyalkyl, OH, CO₂H, cyano, halogen, C₁-C₂ haloalkoxy, amine, and acetamide;
p is from 0 to 4;
R⁴ is selected from hydrogen, C₁-C₂ alkyl, or benzyl; and
n is from 0 to 2.

13. The compound of claim 12, wherein each R^{3a} and R^{3b} is independently selected from halogen, C₁-C₂ alkyl, C₁-C₂ haloalkyl, C₁-C₂ alkoxy, and C₁-C₂ haloalkoxy;
q and q′ are independently from 0 to 2;
J and J′ are each independently selected from phenyl, pyridine, pyrimidine, pyrazine, pyridazine, benzothiazole, benzothiazolone, benzothiadiazole, benzodioxole, benzoxazolone, benzisothiazole, 1-methylpyridin-2(1H)-one, 2,3-dihydrobenzo[b][1,4]dioxine, indazole, benzimidazole, and quinoxaline;
R^{5a} and R^{5b} are each selected from hydrogen, C₁-C₄ alkyl, C₃-C₄ cycloalkyl, C₁-C₂ alkoxy, C₁-C₂ haloalkoxy, C₁-C₂ haloalkyl, methylcarbamate, benzyl, morpholinyl, halide, and CN;
r and r′ are independently selected from 0 to 2; and
R^{6a} and R^{6b} are independently selected from hydrogen, C₁-C₄ alkyl, C₁-C₄ haloalkyl, and C₁-C₄ alkenyl.

14. A compound of Formula IV, including pharmaceutically acceptable salts thereof: 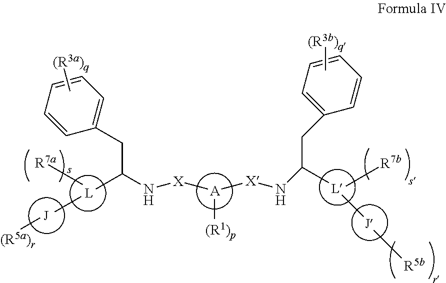
wherein
A is a bond or is selected from C₁-C₅ alkyl, C₂-C₅ alkenyl, C₂-C₅ alkynyl, aryl, C₃-C₆ cycloalkyl, —C₂-C₅ bicycloalkyl, —CO—, —CS—, —C(═N—CN)—, heterocyclyl, nitrogen, sulfur, oxygen, —O—(C₂-C₄ alkyl)-O—, —N(R^{xa})CON(R^{xb})—, and ferrocene;
each R¹ is independently selected from hydrogen, C₁-C₄ alkyl, C₂-C₄ alkenyl, C₁-C₄ alkoxy, C₂-C₄ (alkoxyalkyl), (C₁-C₄ alkoxy)carbonyl, C₁-C₄ alkylthioxy, benzyloxy, C₂-C₄ alkynyl, aryl, carboxylic acid, cyano, halogen, C₁-C₄ haloalkyl, C₁-C₄ haloalkoxy, heterocyclyl, hydroxy, C₁-C₄ hydroxyalkyl, thioxy, —CH₂NH₂, —(C₁-C₄ alkyl)-heteroaryl, —CO—(C₁-C₄ alkyl), —CO(R^{y}), —CON(R^{xa})₂, —NHCON(R^{xa})₂, —NHCO—(C₁-C₄ alkyl), —NHCO₂—(C₁-C₄ alkyl), —NHSO₂—(C₁-C₄ alkyl), —OCH₂-aryl, —SO₂—(C₁-C₄ alkyl), —SO₂—N(R^{xa})₂, —SO₂-heterocyclyl, —N(R^{xa})₂, and nitro;
p is from 0 to 5;
R^{xa} and R^{xb} are independently selected from hydrogen, alkyl, or haloalkyl;
R^{y} is selected from C₁-C₂ dialkylamine or a nitrogen-containing heterocyclyl and is attached to the parent fragment through its nitrogen.
X and X¹ are each are independently a bond or are selected from: 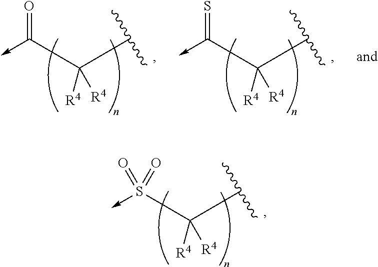
wherein the attachment of X, X¹ to the parent structure is such that the bond with the arrow is oriented toward the respective nitrogen shown in Formula IV; provided, however, that when A is a bond, at least one X or X¹ is not a bond;
each n is independently from 0 to 2;
each R⁴ is independently selected from C₁-C₃ alkyl, C₂-C₃ alkenyl, aryl, aryl(C₁-C₂ alkyl)-, hydroxyl, and halogen, with the option for two R⁴ on same or adjacent carbon(s) to form a ring;
J and J′ are independently a bond or selected from aryl, heterocyclyl, or C₃-C₇ cycloalkyl;
each R^{5a} and R^{5b} is independently selected from hydrogen, C₁-C₄ alkoxy, C₁-C₄ alkyl, C₂-C₄ (alkoxyalkyl), C₃-C₄ cycloalkyl, halogen, C₁-C₄ cycloalkyl, C₁-C₄ haloalkoxy, C₁-C₄ haloalkyl, —CONH₂, —CN, —NHCO(C₁-C₄ alkyl), —NHCON(C₁-C₄ alkyl)₂, —NHCO₂(C₁-C₄ alkyl), —OH, —SO₂N(C₁-C₄ alkyl)₂ and heterocyclyl;
r and r′ are independently from 0 to 4;
L and L′ are independently selected from a five or six-member heteroaryl ring;
each R^{7a} and R^{7b} is independently selected from C₁-C₃ alkoxy, C₁-C₃ alkyl, halogen, C₁-C₃ haloalkoxy, C₁-C₃ haloalkyl, —CONH₂, —CN, —OH, C₂-C₅ alkynol, —NHCO(C₁-C₃ alkyl), —NHCON(C₁-C₃ alkyl)₂, —NHCO₂(C₁-C₃ alkyl), and —SO₂N(C₁-C₃ alkyl)₂, and C₂-C₆ alkyne optionally substituted with 1 to 2 halides;
s and s′ are independently from 0 to 4;
each R^{3a} and R^{3b} is independently selected from C₂-C₄ alkenoxy, C₂-C₄ alkenyl, C₁-C₄ alkoxy, C₂-C₄ (alkoxyalkyl), (C₁-C₄alkoxy)carbonyl, C₁-C₄ alkyl, C₁-C₄ haloalkyl, C₁-C₄ haloalkoxy, carboxyamide, halogen, —CN, —NHCO(C₁-C₄ alkyl), —OH, C₁-C₄ hydroxyalkyl, and —SO₂N-heterocycle; and
q and q′ are independently from 0 to 4;
wherein the attachment of “X”, “X¹” or N to “A” could be on the same or different atom(s) of “A”.

15. The compound as claimed in claim 13, wherein J and J′ are each independently selected from phenyl, pyridine, pyrimidine, pyrazine, pyridazine, benzothiazole, benzothiazolone, benzothiadiazole, benzodioxole, benzoxazolone, benzisothiazole, 1-methylpyridin-2(1H)-one, 2,3-dihydrobenzo[b][1,4]dioxine, indazole, benzimidazole, and quinoxaline;
R^{5a} and R^{5b} are each independently selected from hydrogen, C₁-C₄ alkyl, C₃-C₄ cycloalkyl, C₁-C₂ alkoxy, C₁-C₂ haloalkoxy, C₁-C₂ haloalkyl, methylcarbamate, benzyl, morpholinyl, halide, and CN;
r and r′ are independently from 0 to 2; and
L and L′ are independently selected from a pyridine or an imidazole ring that is attached to the central parental structure through an adjacent carbon atom; and
each of R^{7a} and R^{7b} is independently selected from hydrogen, a C₂-C₅ alkyne that is optionally substituted with 1 to 2 halide, or a C₂-C₅ alkynol.

16. A compound of Formula V, including pharmaceutically acceptable salts thereof: 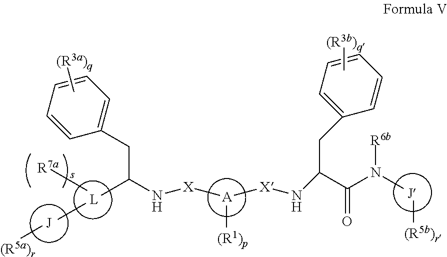
wherein
A is a bond or is selected from C₁-C₅ alkyl, C₂-C₅ alkenyl, C₂-C₅ alkynyl, aryl, C₃-C₆ cycloalkyl, —C₂-C₅ bicycloalkyl, —CO—, —CS—, —C(═N—CN)—, heterocyclyl, nitrogen, sulfur, oxygen, —O—(C₂-C₄ alkyl)-O—, —N(R^{xa})CON(R^{xb})—, and ferrocene;
each R¹ is independently selected from hydrogen, C₁-C₄ alkyl, C₂-C₄ alkenyl, C₁-C₄ alkoxy, C₂-C₄ (alkoxyalkyl), (C₁-C₄ alkoxy)carbonyl, C₁-C₄ alkylthioxy, benzyloxy, C₂-C₄ alkynyl, aryl, carboxylic acid, cyano, halogen, C₁-C₄ haloalkyl, C₁-C₄ haloalkoxy, heterocyclyl, hydroxy, C₁-C₄ hydroxyalkyl, thioxy, —CH₂NH₂, —(C₁-C₄ alkyl)-heteroaryl, —CO—(C₁-C₄ alkyl), —CO(R^{y}), —CON(R^{xa})₂, —NHCON(R^{xa})₂, —NHCO—(C₁-C₄ alkyl), —NHCO₂—(C₁-C₄ alkyl), —NHSO₂—(C₁-C₄ alkyl), —OCH₂-aryl, —SO₂—(C₁-C₄ alkyl), —SO₂—N(R^{xa})₂, —SO₂-heterocyclyl, —N(R^{xa})₂, and nitro;
p is from 0 to 5;
R^{xa} and R^{xb} are independently selected from hydrogen, alkyl, or haloalkyl;
R^{y} is selected from C₁-C₂ dialkylamine or a nitrogen-containing heterocyclyl and is attached to the parent fragment through its nitrogen;
X and X¹ are each are independently a bond or are selected from: 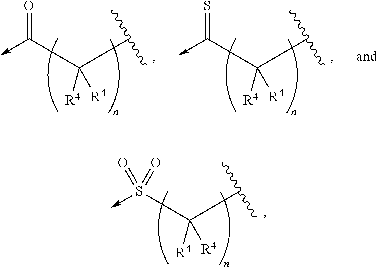
wherein the attachment of X and X¹ to the parent structure is such that the bond with the arrow is oriented toward the respective nitrogen shown in Formula V; provided, however, that when A is a bond, at least one X or X¹ is not a bond;
each n is independently from 0 to 2;
each R⁴ is independently selected from C₁-C₃ alkyl, C₂-C₃ alkenyl, aryl, aryl(C₁-C₂ alkyl)-, hydroxyl, and halogen, with the option for two R⁴ on same or adjacent carbon(s) to form a ring;
J and J′ are independently a bond or selected from aryl, heterocyclyl, or C₃-C₇ cycloalkyl;
R^{5a} and R^{5b} are independently selected from hydrogen, C₁-C₄ alkoxy, C₂-C₄ (alkoxyalkyl), C₁-C₄ alkyl, halogen, C₃-C₄ cycloalkyl, C₁-C₄ haloalkoxy, C₁-C₄ haloalkyl, —CONH₂, —CN, —NHCO(C₁-C₄ alkyl), —NHCON(C₁-C₄alkyl)₂, —NHCO₂(C₁-C₄ alkyl), —OH, —SO₂N(C₁-C₄ alkyl)₂ and heterocyclyl;
r and r′ are independently from 0 to 4;
R^{6b} is selected from hydrogen, C₁-C₄ alkyl, C₁-C₄ alkenyl, and C₃-C₄ cycloalkyl, optionally substituted with halogen, hydroxyl, C₁-C₂ alkoxy, or C₁-C₂ haloalkoxy;
L is selected from a five or six-member heteroaryl ring;
R^{7a} is selected from C₁-C₃ alkoxy, C₁-C₃ alkyl, halogen, C₁-C₃ haloalkoxy, C₁-C₃ haloalkyl, —CONH₂, —CN, OH, C₂-C₅ alkynol, —NHCO(C₁-C₃ alkyl), —NHCON(C₁-C₃ alkyl)₂, —NHCO₂(C₁-C₃ alkyl), and —SO₂N(C₁-C₃ alkyl)₂, and C₂-C₆ alkyne optionally substituted with 1 to 2 halides;
each s is independently from 0 to 4;
each R^{3a} and R^{3b} is independently selected from C₂-C₄ alkenoxy, C₂-C₄ alkenyl, C₁-C₄ alkoxy, C₂-C₄ (alkoxyalkyl), (C₁-C₄alkoxy)carbonyl, C₁-C₄ alkyl, C₁-C₄ haloalkyl, C₁-C₄ haloalkoxy, carboxyamide, halogen, —CN, —NHCO(C₁-C₄ alkyl), —OH, C₁-C₄ hydroxyalkyl, and —SO₂N-heterocycle; and
q and q′ are independently from 0 to 4;
wherein the attachment of “X”, “X¹” or N to “A” could be on the same or different atom(s) of “A”.

17. A compound of Formula VI, including pharmaceutically acceptable salts thereof: 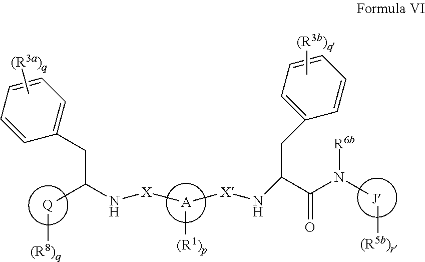
wherein
A is a bond or is selected from C₁-C₅ alkyl, C₂-C₅ alkenyl, C₂-C₅ alkynyl, aryl, C₃-C₆ cycloalkyl, —C₂-C₅ bicycloalkyl, —CO—, —CS—, —C(═N—CN)—, heterocyclyl, nitrogen, sulfur, oxygen, —O—(C₂-C₄ alkyl)-O—, —N(R^{xa})CON(R^{xb})—, and ferrocene;
each R¹ is independently selected from hydrogen, C₁-C₄ alkyl, C₂-C₄ alkenyl, C₁-C₄ alkoxy, C₂-C₄ (alkoxyalkyl), (C₁-C₄ alkoxy)carbonyl, C₁-C₄ alkylthioxy, benzyloxy, C₂-C₄ alkynyl, aryl, carboxylic acid, cyano, halogen, C₁-C₄ haloalkyl, C₁-C₄ haloalkoxy, heterocyclyl, hydroxy, C₁-C₄ hydroxyalkyl, thioxy, —CH₂NH₂, —(C₁-C₄ alkyl)-heteroaryl, —CO—(C₁-C₄ alkyl), —CO(R^{y}), —CON(R^{xa})₂, —NHCON(R^{xa})₂, —NHCO—(C₁-C₄ alkyl), —NHCO₂—(C₁-C₄ alkyl), —NHSO₂—(C₁-C₄ alkyl), —OCH₂-aryl, —SO₂—(C₁-C₄ alkyl), —SO₂—N(R^{xa})₂, —SO₂-heterocyclyl, —N(R^{xa})₂, and nitro;
p is from 0 to 5;
R^{xa} and R^{xb} are independently selected from hydrogen, alkyl, or haloalkyl;
R^{y} is selected from C₁-C₂ dialkylamine or a nitrogen-containing heterocyclyl and is attached to the parent fragment through its nitrogen;
X and X¹ are each are independently a bond or are selected from: 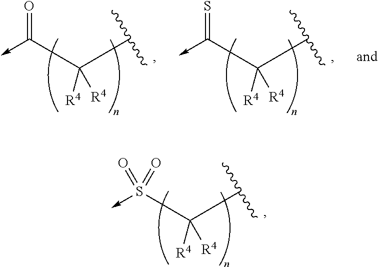
wherein the attachment of X and X¹ to the parent structure is such that the bond with the arrow is oriented toward the respective nitrogen shown in Formula VI; provided, however, that when A is a bond, at least one X or X¹ is not a bond;
each n is independently from 0 to 2;
each R⁴ is independently selected from C₁-C₃ alkyl, C₁-C₃ alkenyl, aryl, aryl(C₁-C₂ alkyl)-, hydroxyl, and halogen, with the option for two R⁴ on same or adjacent carbon(s) to form a ring;
J′ is a bond or is selected from aryl, heterocyclyl, or C₃-C₇ cycloalkyl;
R^{5b} is selected from hydrogen, C₁-C₄ alkoxy, C₁-C₄ alkyl, C₂-C₄ (alkoxyalkyl), C₃-C₄ cycloalkyl, halogen, C₁-C₄ haloalkoxy, C₁-C₄ haloalkyl, —CONH₂, —CN, —NHCO(C₁-C₄ alkyl), —NHCON(C₁-C₄ alkyl)₂, —NHCO₂(C₁-C₄ alkyl), —OH, —SO₂N(C₁-C₄ alkyl)₂ and heterocyclyl;
r′ is from 0 to 4;
R^{6b} is selected from hydrogen, C₁-C₄ alkyl, C₂-C₄ alkenyl, and C₃-C₄ cycloalkyl, optionally substituted with halogen, hydroxyl, C₁-C₂ alkoxy, or C₁-C₂ haloalkoxy;
Q is a bond or is selected from heterocycle and a —CON(C₁-C₃ alkyl)₂ with the option for the two alkyl groups together with the nitrogen atom to which they are attached to form a heterocycle;
R⁸ is selected from hydrogen, C₁-C₂ alkyl and C₁-C₂ alkyl-S—;
each R^{3a} and R^{3b} is independently selected from C₂-C₄ alkenoxy, C₂-C₄ alkenyl, C₁-C₄ alkoxy, C₂-C₄ (alkoxyalkyl), (C₁-C₄alkoxy)carbonyl, C₁-C₄ alkyl, C₁-C₄ haloalkyl, C₁-C₄ haloalkoxy, carboxyamide, halogen, —CN, —NHCO(C₁-C₄ alkyl), —OH, C₁-C₄ hydroxyalkyl, and —SO₂N-heterocycle; and
q and q′ are independently from 0 to 2;
wherein the attachment of “X”, “X¹” or N to “A” could be on the same or different atom(s) of “A”.

18. A composition comprising a compound of claim 1 and a pharmaceutically acceptable carrier, excipient, and/or diluent.

19. A method of treating method of treating HIV infection comprising administering a therapeutically effective amount of a compound of claim 1 to a patient.
